(19) United States
(12) Patent Application Publication
(10) Pub. No.: US 2024/0041042 A1
(43) Pub. Date: Feb. 8, 2024

(54) 1-(PYRIDYL)-5-AZINYLPYRAZOLE DERIVATIVES, AND THEIR USE FOR CONTROL OF UNDESIRED PLANT GROWTH

(71) Applicant: Bayer Aktiengesellschaft, Leverkusen (DE)

(72) Inventors: Estella BUSCATO, Sant Cugat del Valles (ES); Michael Gerhard HOFFMANN, Konstanz (DE); Harald JAKOBI, Frankfurt (DE); Thomas MÜLLER, Frankfurt (DE); Birgit BOLLENBACH-WAHL, Weiler/Bingen (DE); Jan DITTGEN, Frankfurt (DE); Elmar GATZWEILER, Bad Nauheim (DE); Anna Maria REINGRUBER, Heppenheim (DE)

(21) Appl. No.: 18/249,310

(22) PCT Filed: Oct. 19, 2021
(86) PCT No.: PCT/EP2021/078878

§ 371 Date: Apr. 17, 2023

(30) Foreign Application Priority Data
Oct. 23, 2020 (EP) 20203487.2

(51) Int. Cl. A01N 43/56; A01N 43/66; A01N 43/60; A01N 43/58; A01N 43/54

(52) U.S. Cl. CPC A01N 43/56; A01N 43/66; A01N 43/60; A01N 43/58; A01N 43/54

(57) ABSTRACT

The present invention relates to novel herbicidally active substituted 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids, and to the derivatives thereof according to the general formula (I) or agrochemically acceptable salts thereof. The present invention further relates to processes for preparing these compounds and to the use thereof for controlling broad-leaved weeds and weed grasses.

The invention relates to the technical field of crop protection compositions, particularly to that of herbicides for control of broad-leaved weeds and weed grasses in crops of useful plants and in the ornamental garden sector and for general control of broad-leaved weeds and weed grasses in areas of the environment where plant growth is disruptive.

The invention relates to novel substituted 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids, and derivatives thereof, processes for preparation thereof and the use thereof for control of harmful plants.

It is a feature of the 1-(pyridyl)-5-azinylpyrazoles of the invention that they have a further substituent in the 4 position of the pyrazole ring.

The 1-pyridyl-5-azinylpyrazolyl-3-oxyalkyl acids of the invention and the derivatives thereof differ from the already known 1,5-diphenylpyrazolyl-3-oxoacetic acids in that they have a pyridyl radical (Q1-Q3) in the 1 position and a variable azinyl radical (A1-A15) in the 5 position of the pyrazole ring.

The prior art discloses biological effects of substituted 1,5-diphenylpyrazolyl-3-oxoacetic acids and processes for preparing these compounds. DE 2828529 A1 describes the preparation and the lipid-lowering action of 1,5-diphenylpyrazolyl-3-oxoacetic acids.

CN 101284815 A discloses 1,5-diphenylpyrazolyl-3-oxoacetic acids as bactericidally active agrochemicals. Journal of Heterocyclic Chemistry (2012), 49(6), 1370-1375 describes further syntheses and the fungicidal action of 1,5-diphenylpyrazolyl-3-oxoacetic acids.

WO 2008/083233 A2 describes 1,5-diphenylpyrazolyl-3-oxyalkyl acids substituted in the 4 position of the pyrazole and derivatives thereof as substances that are suitable for breaking up cell aggregates. Ethyl [(4-chloro-1,5-diphenyl-1H-pyrazol-3-yl)oxy]acetate is specifically disclosed.

In addition, the synthesis of some 4-chloro-1,5-diphenylpyrazolyl-3-oxyacetic acids and ethyl esters thereof is described in European Journal of Organic Chemistry (2011), 2011 (27), 5323-5330.

By contrast, 1-pyridyl-5-azinylpyrazolyl-3-oxyacetic acids, 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids, and derivatives thereof, are unknown to date.

It is an object of the present invention to provide novel pyrazole derivatives, namely 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids, which can be used as herbicides or plant growth regulators, having satisfactory herbicidal action and a broad spectrum of activity against harmful plants.

The object is achieved by substituted 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids that feature a pyridyl radical in the 1 position, an azinyl radical in the 5 position and a further substituent in the 4 position of the pyrazole ring, and have very good herbicidal action.

Surprisingly, these compounds are highly effective against a broad range of economically important weed grasses and broadleaved weeds.

The present invention provides substituted 1-pyridyl-5-azinylpyrazolyl-3-oxyacetic acids, 1-pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids and 1-pyridyl-5-azinylpyrazolyl-3-sulfonylalkyl acids of the general formula (I)

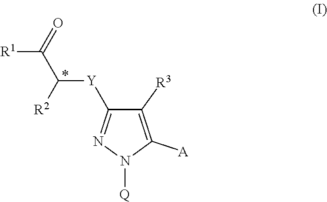

and their agrochemically acceptable salts, N-oxides, hydrates and hydrates of the salts and N-oxides, where
A is selected from the group consisting of A1-A15:

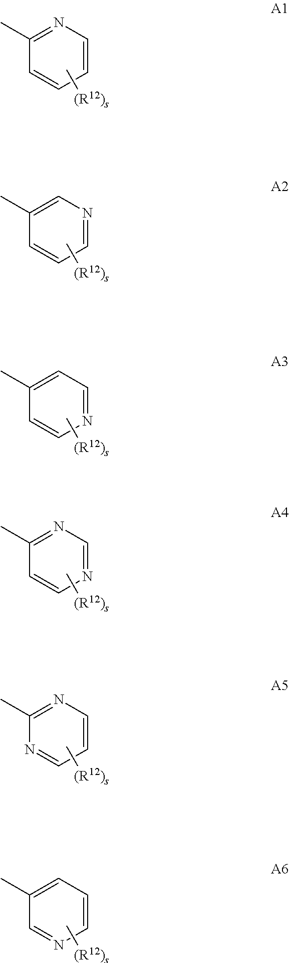

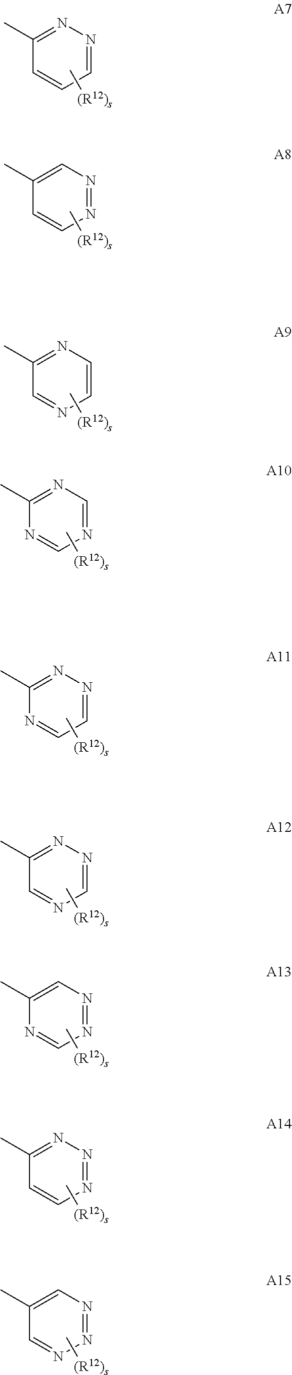

Q is selected from the group consisting of Q1-Q3:

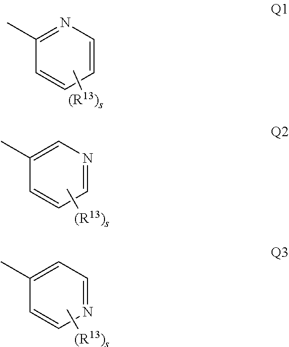

R¹ is —OR^{1a}, —NR⁹R¹⁰ or —O(CH₂)ₓO(CH₂)_{y}O(CH₂)_{z}H;
R^{1a} is hydrogen or
   is (C₁-C₆)-alkyl, (C₃-C₆)-cycloalkyl, each of which is unsubstituted or substituted by one or more substituents selected from the group consisting of halogen, (C₁-C₆)-alkyl, (C₁-C₆)-haloalkyl, (C₃-C₆)-cycloalkyl, (C₁-C₆)-alkoxy, (C₁-C₂)-haloalkoxy, (C₁-C₆)-alkoxycarbonyl, cyano and nitro
   or is (C₂-C₄)-alkenyl, CH₂C(COOMe)=CH₂, (C₂-C₄)-alkynyl or is (C₁-C₆)-alkyl-S—(C₁-C₆)-alkyl-, (C₁-C₆)-alkyl-SO—(C₁-C₆)-alkyl-, (C₁-C₆)-alkyl-SO₂—(C₁-C₆)-alkyl- or is —N═(C₃-C₆)-cycloalkyl, —N═C(CH₃)₂ or
   is heterocyclyl, heteroaryl, aryl or
   is heterocyclyl-(C₁-C₄)-alkyl-, heteroaryl-(C₁-C₄)-alkyl-, aryl-(C₁-C₄)-alkyl-, each of which is unsubstituted or substituted by one or more substituents selected from the group consisting of halogen, (C₁-C₆)-alkyl, (C₁-C₆)-haloalkyl;
R⁹ is hydrogen, (C₁-C₁₂)-alkyl;
R¹⁰ is hydrogen, aryl, heteroaryl, heterocyclyl, (C₁-C₁₂)-alkyl, (C₃-C₈)-cycloalkyl, (C₃-C₈)-cycloalkyl-(C₁-C₇)-alkyl-, (C₂-C₁₂)-alkenyl, (C₅-C₇)-cycloalkenyl, (C₂-C₁₂)-alkynyl, S(O)ₙR⁵, cyano, nitro, OR⁵, SO₂NR⁶R⁷, CO₂R⁸, COR⁸, NR⁶R⁸, NR⁶COR⁸, NR⁶CO₂R⁸, NR⁶SO₂R⁸, where the abovementioned alkyl, cycloalkyl, alkenyl, cycloalkenyl and alkynyl radicals are unsubstituted or each independently substituted by “m” radicals selected from the group consisting of hydrogen, optionally mono- or polysubstituted aryl, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or
R⁹ and R¹⁰ together with the nitrogen atom to which they are bonded form a saturated or partly or fully unsaturated five-, six- or seven-membered ring which is optionally mono- to hexasubstituted by the following radicals from the group consisting of halogen, (C₁-C₆)-alkyl, (C₁-C₆)-haloalkyl, OR⁵, S(O)ₙR⁵, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl, aryl;
R⁶ is hydrogen, (C₁-C₆)-alkyl, (C₃-C₆)-cycloalkyl, (C₁-C₆)-haloalkyl, aryl;
R⁷ is hydrogen, (C₁-C₆)-alkyl, (C₃-C₆)-cycloalkyl, (C₃-C₄)-alkenyl, (C₃-C₄)-alkynyl, CH₂—C(O)O(C₁-C₂)-alkyl;
R⁸ is hydrogen, (C₁-C₆)-alkyl, (C₃-C₆)-cycloalkyl, (C₃-C₄)-alkenyl, (C₁-C₆)-alkyl-C(O)O(C₁-C₂)-alkyl or (C₃-C₄)-alkynyl;
R² is hydrogen, cyano, (C₁-C₆)-alkyl, (C₁-C₆)-alkoxy-(C₁-C₆)-alkyl-, (C₁-C₆)-haloalkyl, (C₁-C₆)-alkoxy, (C₂-C₆)-alkenyl, (C₂-C₆)-haloalkenyl, (C₂-C₆)-alkynyl, (C₂-C₆)-haloalkynyl, (C₃-C₆)-cycloalkyl;
R³ is halogen, cyano, isocyano, NO₂, (C₃-C₆)-cycloalkyl, (C₁-C₆)-alkyl, (C₃-C₆)-halocycloalkyl, (C₁-C₆)-haloalkyl, (C₁-C₆)-alkylcarbonyl-, (C₁-C₆)-haloalkylcarbonyl-, (C₁-C₆)-alkyloxycarbonyl-, (C₂-C₃)-alkenyl, (C₂-C₃)-haloalkenyl, (C₂-C₃)-alkynyl, (C₂-C₃)-haloalkynyl, (C₁-C₆)-alkyl-S(O)ₙ and (C₁-C₆)-haloalkyl-S(O)ₙ, CHO, NH₂;
R¹² is halogen, cyano, isocyano, NO₂, (C₁-C₆)-alkyl, (C₁-C₆)-haloalkyl, (C₁-C₆)-alkylcarbonyl, (C₁-C₆)-haloalkylcarbonyl, (C₁-C₆)-alkyloxycarbonyl, (C₁-C₆)-alkoxy, (C₁-C₆)-haloalkoxy, (C₁-C₆)-alkyl-, S(O)ₙ, (C₂-C₃)-alkenyl, (C₂-C₃)-haloalkenyl, (C₂-C₃)-alkynyl, (C₂-C₃)-haloalkynyl, NH₂;
R¹³ is halogen, cyano, isocyano, NO₂, (C₁-C₆)-alkyl, (C₁-C₆)-haloalkyl, (C₁-C₆)-alkylcarbonyl, (C₁-C₆)-haloalkylcarbonyl, (C₁-C₆)-alkyloxycarbonyl, (C₁-C₆)-alkoxy, (C₁-C₆)-haloalkoxy, (C₁-C₆)-alkyl-S(O)ₙ, (C₂-C₃)-alkenyl, (C₂-C₃)-haloalkenyl; (C₂-C₃)-alkynyl, (C₂-C₃)-haloalkynyl, NH₂;
Y is oxygen, S(O)ₙ;
m is 0, 1 or 2;
n is 0, 1 or 2;
o is 0, 1 or 2;
p is 0 or 1;
q is 0 or 1;
r is 3, 4, 5 or 6;
s is 0, 1, 2, 3 or 4;
x is 2, 3;
y is 2, 3;
z is 1, 2.

There follows a description of preferred, particularly preferred, very particularly preferred and most preferred definitions of each of the individual substituents. The other substituents of the general formula (I) which are not specified hereinafter have the definition given above.

This results in various embodiments for the compound of the general formula (I).

Not encompassed are combinations which contravene the laws of nature and which the person skilled in the art would therefore rule out on the basis of their knowledge.

Preference is given to compounds of the general formula (I) and their agrochemically compatible salts, N-oxides, hydrates, and hydrates of the salts and N-oxides, in which

A is selected from the group consisting of A1-A4, A6, A8, A9, A12, A15

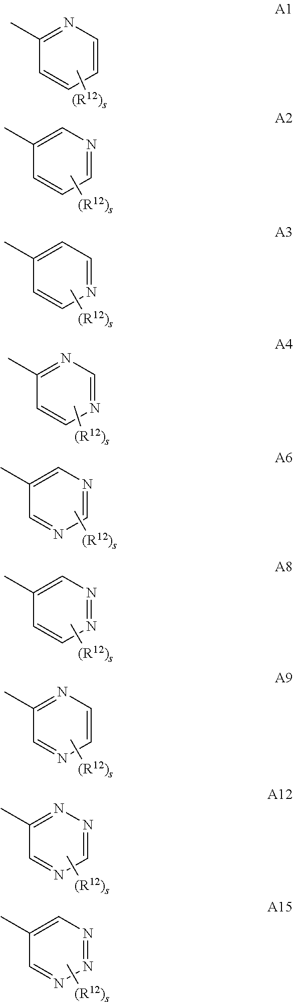

Q is selected from the group consisting of Q1-Q3:

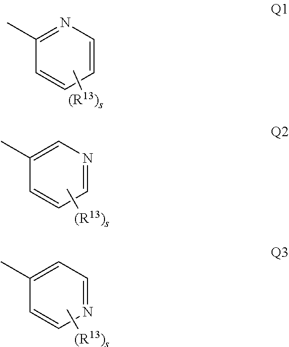

R¹ is —OR^{1a}, —NR⁹R¹⁰ or —O(CH₂)ₓO(CH₂)_{y}O(CH₂)_{z}H;
R^{1a} is hydrogen or
   is (C₁-C₃)-alkyl, (C₃-C₆)-cycloalkyl, each of which is unsubstituted or substituted by one or more substituents selected from the group consisting of halogen, (C₁-C₄)-alkyl, (C₁-C₄)-haloalkyl, (C₃-C₆)-cycloalkyl, (C₁-C₄)-alkoxy, (C₁-C₄)-alkoxycarbonyl, cyano,
   or is (C₂-C₄)-alkenyl, (C₂-C₄)-alkynyl, or
   is (C₂-C₃)-alkyl-SMe-, (C₂-C₃)-alkyl-SOMe-, (C₂-C₃)-alkyl-SO₂Me-, or
   is —N═C(CH₃)₂ or
   is heterocyclyl, or
   is heterocyclyl-(C₁-C₂)-alkyl-, heteroaryl-(C₁-C₂)-alkyl-, aryl-(C₁-C₂)-alkyl-, each of which is unsubstituted or substituted by one or more substituents selected from the group consisting of halogen, (C₁-C₂)-alkyl, (C₁-C₂)-haloalkyl;
R⁹ is hydrogen, (C₁-C₄)-alkyl;
R¹⁰ is (C₁-C₄)-alkyl, (C₂-C₄)-alkenyl, (C₂-C₄)-alkynyl, S(O)ₙR⁵, SO₂NR⁶R⁷, where the abovementioned alkyl, alkenyl and alkynyl radicals are unsubstituted or each independently substituted by “m” radicals selected from the group consisting of halogen, cyano, S(O)ₙR⁵, SO₂NR⁶R⁷, CO₂R⁸, CONR⁶R⁸,
or
R⁹ and R¹⁰ together with the nitrogen atom to which they are bonded form a saturated or partly or fully unsaturated five-, six- or seven-membered ring which is optionally mono- or disubstituted by the following radicals from the group consisting of (C₁-C₄)-alkyl, (C₁-C₄)-haloalkyl, CO₂R⁸ and CONR⁶R⁸;
R⁵ is (C₁-C₄)-alkyl, (C₃-C₆)-cycloalkyl, (C₁-C₄)-haloalkyl, phenyl;
R⁶ is hydrogen, (C₁-C₄)-alkyl, (C₃-C₆)-cycloalkyl, (C₁-C₄)-haloalkyl, phenyl;
R⁷ is hydrogen, (C₁-C₄)-alkyl, (C₃-C₆)-cycloalkyl, (C₃-C₄)-alkenyl, (C₃-C₄)-alkynyl;
R⁸ is hydrogen, (C₁-C₄)-alkyl, (C₃-C₆)-cycloalkyl, (C₃-C₄)-alkenyl, (C₁-C₄)-alkyl-C(O)O(C₁-C₂)-alkyl or (C₃-C₄)-alkynyl;
R² is hydrogen, (C₁-C₃)-alkyl, (C₁-C₃)-alkoxy, (C₂-C₃)-alkenyl, (C₂-C₃)-alkynyl, (C₃-C₆)-cycloalkyl;
R³ is halogen, cyano, isocyano, NO₂, (C₃-C₆)-cycloalkyl, (C₁-C₄)-alkyl, (C₃-C₆)-halocycloalkyl, (C₁-C₄)-haloalkyl, (C₁-C₄)-alkylcarbonyl-, (C₁-C₄)-haloalkylcarbonyl-, (C₁-C₄)-alkyloxycarbonyl-, (C₂-C₃)-alkenyl, (C₂-C₃)-haloalkenyl, (C₂-C₃)-alkynyl, (C₂-C₃)-haloalkynyl, CHO;
R¹² is halogen, cyano, NO₂, (C₁-C₄)-alkyl, (C₁-C₄)-haloalkyl, (C₁-C₄)-alkylcarbonyl, (C₁-C₄)-haloalkylcarbonyl, (C₁-C₄)-alkyloxycarbonyl, (C₁-C₄)-alkoxy, (C₁-C₄)-haloalkoxy, (C₂-C₃)-alkenyl, (C₂-C₃)-haloalkenyl, (C₂-C₃)-alkynyl, (C₂-C₃)-haloalkynyl, NH₂;
R¹³ is halogen, cyano, NO₂, (C₁-C₄)-alkyl, (C₁-C₄)-haloalkyl, (C₁-C₄)-alkylcarbonyl, (C₁-C₄)-haloalkylcarbonyl, (C₁-C₄)-alkyloxycarbonyl, (C₁-C₄)-alkoxy, (C₁-C₄)-haloalkoxy, (C₂-C₃)-alkenyl, (C₂-C₃)-haloalkenyl; (C₂-C₃)-alkynyl, (C₂-C₃)-haloalkynyl, NH₂;
Y is oxygen, S(O)ₙ;
m is 0 or 1;
n is 0, 1 or 2;
s is 0, 1, 2 or 3;
x is 2;
y is 2;
z is 1.

Particular preference is given to compounds of the general formula (I) and their agrochemically compatible salts, N-oxides, hydrates, and hydrates of the salts and N-oxides, in which
A is selected from the group consisting of A2, A3, A6 and A8

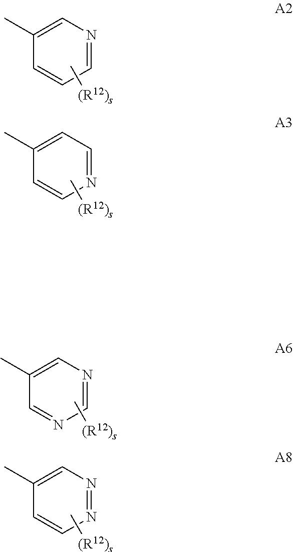

Q is selected from the group consisting of Q1-Q3:

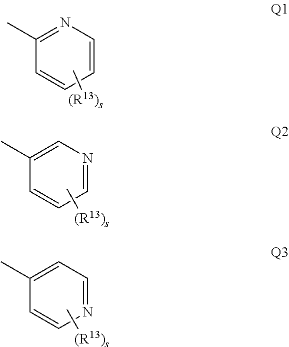

R¹ is —OR^{1a}, —NR⁹R¹⁰ or —O(CH₂)₂O(CH₂)₂OCH₃;
R^{1a} is hydrogen or
   is (C₁-C₃)-alkyl which is unsubstituted or substituted by a substituent selected from the group consisting of —C(O)OMe, cyclopropyl, cyclopentyl, methoxy, cyano, trifluoromethyl or
   is (C₃-C₆)-cycloalkyl or
   is —N═C(CH₃)₂ or
   is prop-2-ynyl or
   is oxetan-3-yl-(C₁-C₂)-alkyl-, tetrahydrofuran-2-yl-(C₁-C₂)-alkyl-, tetrahydrofuran-3-yl-(C₁-C₂)-alkyl-, pyridin-2-yl-(C₁-C₂)-alkyl-, pyridin-3-yl-(C₁-C₂)-alkyl-, pyridin-4-yl-(C₁-C₂)-alkyl-, phenyl-(C₁-C₂)-alkyl-;
R⁹ is hydrogen;
R¹⁰ is (C₁-C₄)-alkyl where the alkyl radical is unsubstituted or monosubstituted by CO₂R⁸, S(O)₂R⁵ or SO₂NR⁶R⁷;
or
R⁹ and R¹⁰ together with the nitrogen atom to which they are bonded form a saturated five- or seven-membered ring monosubstituted by CO₂R⁸;
R⁵ is methyl, ethyl or CH₂CF₃;
R⁶ is hydrogen or methyl;
R⁷ is methyl or ethyl;
R⁸ is hydrogen, methyl or ethyl;
R² is hydrogen, methyl or ethyl;
R³ is halogen, cyano, NO₂, (C₃-C₅)-cycloalkyl, (C₃-C₅)-halocycloalkyl, (C₁-C₂)-alkyl, (C₁-C₂)-haloalkyl;
R¹² is fluorine or chlorine;
R¹³ is fluorine, chlorine, methyl, ethyl, methoxy, trifluoromethyl;
Y is oxygen;
s is 0, 1, 2.

Very particular preference is given to compounds of the general formula (I) and their agrochemically compatible salts, N-oxides, hydrates, and hydrates of the salts and N-oxides, in which
A is selected from the group consisting of A2, A3 and A6

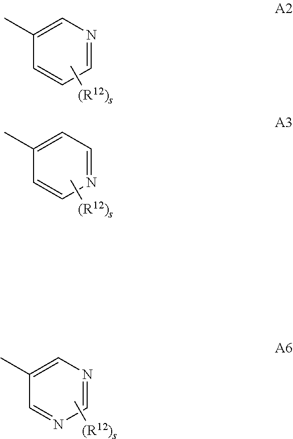

Q is selected from the group consisting of Q1-Q3:

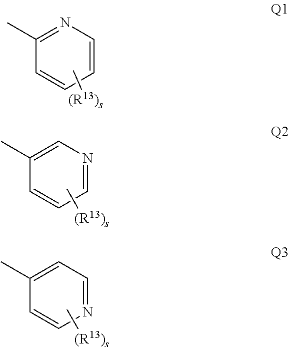

R¹ is —OR^{1a} or —NR⁹R¹⁰,
R^{1a} is hydrogen or
   methyl, ethyl, each of which is unsubstituted or substituted by a substituent selected from the group consisting of —C(O)OMe, cyclopropyl, cyclopentyl, methoxy, cyano, trifluoromethyl or
   is —N═C(CH₃)₂ or
   is prop-2-ynyl or
   —CH₂-tetrahydrofuran-2-yl, —CH₂-tetrahydrofuran-3-yl, —CH₂-oxetan-3-yl, —CH₂-pyridyl-2-yl, —CH₂-pyridin-3-yl, —CH₂-pyridin-4-yl, —CH₂-phenyl,
R⁹ is hydrogen;
R¹⁰ methyl or ethyl, where the radicals are monosubstituted by CO₂R⁸,
or
R⁹ and R¹⁰ together with the nitrogen atom to which they are bonded form a cyclopentyl or cyclohexyl radical monosubstituted by CO₂R⁸;
R⁸ is methyl or ethyl;
R² is hydrogen or methyl;
R³ is halogen, cyano, NO₂, cyclopropyl, cyclobutyl, 2,2-difluorocyclopropyl, trifluoromethyl;
R¹² is fluorine;
R¹³ is fluorine, chlorine, methyl, trifluoromethyl;
Y is oxygen;
s is 0, 1, 2.

The most preference is given to compounds of the general formula (I) and their agrochemically compatible salts, N-oxides, hydrates, and hydrates of the salts and N-oxides, in which
A is selected from the group consisting of A2, A3 and A6

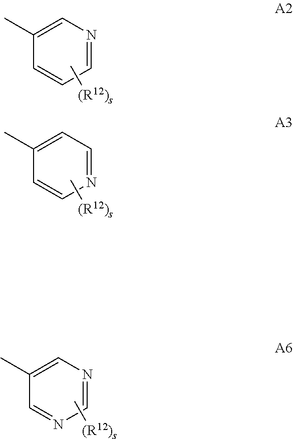

Q is selected from the group consisting of Q1-Q3:

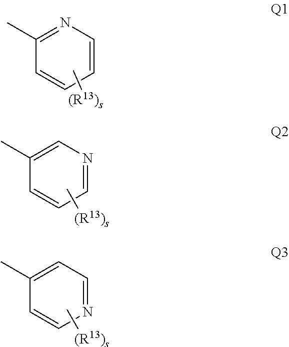

R¹ is —OR^{1a};
R^{1a} is hydrogen, methyl, ethyl;
R² is hydrogen or methyl;
R³ is fluorine, chlorine, bromine, cyano, NO₂, cyclopropyl, trifluoromethyl;
R¹² is fluorine;
R¹³ is fluorine, chlorine, trifluoromethyl;
Y is oxygen;
s is 0, 1, 2.

The present invention further provides compounds of the formula (Z) with Q=Q1, R¹=—OR^{1a} and Y═O

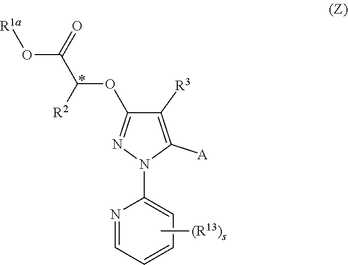

where the above-described definitions are applicable, including all preferred, particularly preferred, very particularly preferred and most preferred definitions.

The present invention further provides compounds of the formula (Y) with Q=Q1, Y═O and A=A2

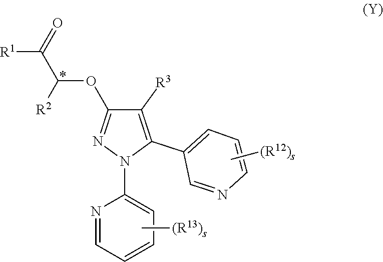

where the above-described definitions are applicable, including all preferred, particularly preferred, very particularly preferred and most preferred definitions.

The present invention further provides acids of the formula (Ia)

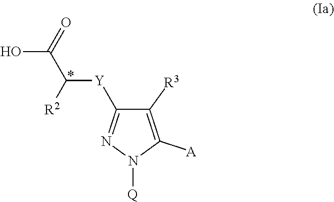

where the above-described definitions are applicable, including all preferred, particularly preferred, very particularly preferred and most preferred definitions.

The present invention further provides compounds of the formula (V)

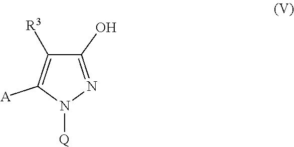

where the above-described definitions are applicable, including all preferred, particularly preferred, very particularly preferred and most preferred definitions.

The present invention further provides compounds of the formula (VI)

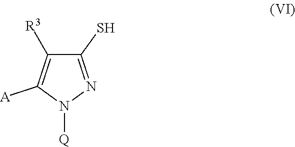

where the above-described definitions are applicable, including all preferred, particularly preferred, very particularly preferred and most preferred definitions.

Alkyl denotes saturated straight-chain or branched hydrocarbyl radicals having the number of carbon atoms specified in each case, e.g. C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Halogen-substituted alkyl denotes straight-chain or branched alkyl groups where some or all of the hydrogen atoms in these groups may be replaced by halogen atoms, e.g. C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

Alkenyl denotes unsaturated straight-chain or branched hydrocarbyl radicals having the number of carbon atoms stated in each case and one double bond in any position, for example C₂-C₆-alkenyl such as ethenyl (vinyl), 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl denotes straight-chain or branched hydrocarbyl radicals having the number of carbon atoms specified in each case and one triple bond in any position, e.g. C₂-C₆-alkynyl such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Cycloalkyl denotes a carbocyclic saturated ring system having preferably 3 to 6 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene.

In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl (norbornyl), adamantan-1-yl and adamantan-2-yl.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

Cycloalkenyl denotes a carbocyclic, nonaromatic, partially unsaturated ring system having preferably 4-8 carbon atoms, e.g., 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

Alkoxy denotes saturated straight-chain or branched alkoxy radicals having the number of carbon atoms specified in each case, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Halogen-substituted alkoxy denotes straight-chain or branched alkoxy radicals having the number of carbon atoms specified in each case, where some or all of the hydrogen atoms in these groups may be replaced by halogen atoms as specified above, e.g. C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-1,2-difluoroethoxy, 2,2-dichloro-2-fluoroethoxy, 2,2,2-trichloroethoxy, pentafluoroethoxy and 1,1,1-trifluoroprop-2-oxy.

Heterocyclyl denotes a saturated or partially unsaturated mono-, bi- or tricyclic ring system group of carbon atoms and at least one heteroatom, preferably selected from N, O and/or S.

Heteroaryl, unless defined differently elsewhere: a mono-, bi- or tricyclic heterocyclic group of carbon atoms and at least one heteroatom, where at least one cycle is aromatic. In one embodiment, at least one heteroatom is N, O or S. In one embodiment, all heteroatoms are selected from N, O or S. In one embodiment, the ring system is a 5- to 10- or a 5- to 6-membered ring system. In one embodiment, heteroaryl is an aromatic monocyclic ring system of 5 or 6 ring atoms. In a further embodiment, heteroaryl is an aromatic monocyclic ring system containing 1 to 4 heteroatoms from the group N, O or S. Furthermore, heteroaryl may be a bicyclic ring system consisting of 8 to 14 ring atoms or a tricyclic ring system consisting of 13 to 14 ring atoms. Examples: furyl, thienyl, pyrazolyl, imidazolyl, triazolyl, thiazolyl, indolyl, benzimidazolyl, indazolyl, benzofuranyl, benzothiophenyl, benzothiazolyl, benzoxazolyl, quinolinyl, isoquinolinyl.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”.

The aryls listed above are preferably independently mono- to pentasubstituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

The term “halogen” denotes fluorine (abbreviation: F), chlorine (abbreviation: Cl), bromine (abbreviation: Br) or iodine (abbreviation: I). If the term is used for a radical, “halogen” means a fluorine, chlorine, bromine or iodine atom.

The substituent cyano may be abbreviated to “CN”, the substituent nitro may be abbreviated to “NO₂”, the substituent methyl may be abbreviated to “Me”, the substituent ethyl may be abbreviated to “Et”, the substituent propyl may be abbreviated to “Pr”, the substituent iso-propyl (or i-propyl) may be abbreviated to “iPr”, the substituent n-propyl may be abbreviated to “nPr”, and the substituent trifluoromethyl may be abbreviated to “CF₃”. Further substituents may be abbreviated analogously.

According to the nature of the substituents defined above, the compounds of the formula (I) have acidic properties and are able to form salts, and if appropriate also internal salts or adducts, with inorganic or organic bases or with metal ions. If the compounds of the formula (I) bear hydroxyl, carboxyl or other groups which induce acidic properties, these compounds can be reacted with bases to give salts. Suitable bases are, for example, hydroxides, carbonates, hydrogencarbonates of the alkali metals and alkaline earth metals, especially those of sodium, potassium, magnesium and calcium, and also ammonia, primary, secondary and tertiary amines having (C₁-C₄)-alkyl groups, mono-, di- and trialkanolamines of (C₁-C₄)-alkanols, choline and chlorocholine, and organic amines, such as trialkylamines, morpholine, piperidine or pyridine. These salts are compounds in which the acidic hydrogen is replaced by an agriculturally suitable cation, for example metal salts, especially alkali metal salts or alkaline earth metal salts, especially sodium and potassium salts, or else ammonium salts, salts with organic amines or quaternary ammonium salts, for example with cations of the formula [NRR′R″R′″]⁺ in which R to R′″ are each independently an organic radical, especially alkyl, aryl, aralkyl or alkylaryl. Also useful are alkylsulfonium and alkylsulfoxonium salts, such as (C₁-C₄)-trialkylsulfonium and (C₁-C₄)-trialkylsulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid, or sulfonic acids, for example p-toluenesulfonic acid, onto a basic group, for example amino, alkylamino, dialkylamino, piperidino, morpholino or pyridino. These salts then contain the conjugate base of the acid as anion.

Suitable substituents present in deprotonated form, for example sulfonic acids or carboxylic acids, are capable of forming inner salts with groups, such as amino groups, which can be protonated for their part.

If a group is polysubstituted by radicals, this means that this group is substituted by one or more identical or different radicals from those mentioned.

The present compounds of the general formula (I) have, at the second carbon of the alkyl acid structure, a chiral carbon atom which, in the structure shown below, is indicated by the marker (*):

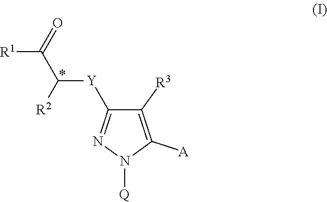

According to the rules of Cahn, Ingold and Prelog (CIP rules), this carbon atom can have either an (R) configuration or an (S) configuration.

The present invention encompasses compounds of the general formula (I) both with (S) and with (R) configuration, meaning that the present invention encompasses the compounds of the general formula (I) in which the carbon atom in question has
(1) an (R) configuration; or
(2) an (S) configuration.

Furthermore, the scope of the present invention also encompasses any mixtures of compounds of the general formula (I) having an (R) configuration (compounds of the general formula (I-(R)) with compounds of the general formula (I) having an (S) configuration (compounds of the general formula (I-S)), the present invention also encompassing a racemic mixture of the compounds of the general formula (I) having (R) and (S) configuration.

However, within the context of the present invention, preference is given particularly to compounds of the general formula (I) having (R) configuration with a selectivity of 60% to 100%, preferably 80% to 100%, especially 90% to 100%, very particularly 95% to 100%, where the particular (R) compound is present with an enantioselectivity of in each case more than 50% ee, preferably 60% to 100% ee, especially 80% to 100% ee, very particularly 90% to 100% ee, most preferably 95% to 100% ee, based on the total content of (R) compound in question.

The present invention therefore relates more particularly to compounds of the general formula (I*) in which the stereochemical configuration on the carbon atom marked by (*) is present with a stereochemical purity of 60% to 100% (R), preferably 80% to 100% (R), especially 90% to 100% (R), very particularly 95% to 100% (R).

Taking account of the Cahn, Ingold and Prelog rule, at the carbon atom marked by (*) there may however also be a situation in which, owing to the priority of the substituents in question, the (S) configuration is preferred at the carbon atom marked by (*). This is the case, for example, when the R² radical corresponds to a (C₁-C₆)-alkoxy radical.

Therefore, within the scope of the present invention, preference is given especially to compounds of the general formula (I) whose spatial arrangement corresponds to those compounds of the general formula (I) with R²=methyl having (R) configuration with a selectivity of 60% to 100%, preferably 80% to 100%, especially 90% to 100%, very particularly 95% to 100%, where the respective (R) analogue compound is present with an enantioselectivity of in each case more than 50% ee, preferably 60% to 100% ee, especially 80% to 100% ee, very particularly 90% to 100% ee, most preferably 95% to 100% ee, based on the total content of (R) analogue compound in question. Therefore, the present invention relates more particularly to compounds of the general formula (I) in which the stereochemical configuration on the carbon atom marked by (*) is present with a stereochemical purity of 60% to 100% (R or R analogue), preferably 80% to 100% (R or R analogue), especially 90% to 100% (R or R analogue), very particularly 95% to 100% (R or R analogue).

In addition, depending on the respective radicals chosen, further stereoelements may be present in the inventive compounds of the general formula (I).

Preference is given to the compounds listed in the tables below.

The compounds of the general formula (I) having (R) configuration are marked accordingly in the column which lists the radical R². For example, if R²=methyl (Me), the preferred stereochemical configuration at the carbon atom marked by (*) of the general formula (I) is the (R) configuration.

TABLE I
Q = Q1 in formula (I)
The linkage of R¹ is indicated either by “-” or “#”.

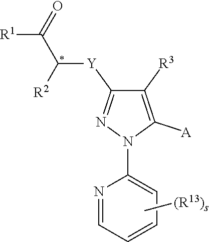

| Ex. no. | R¹ | R² | R³ | A | Y | R¹³ | s |
|---|---|---|---|---|---|---|---|
| I-01 | —OEt | H | Br | 2-fluoropyridin-4-yl | O | — | 0 |
| I-02 | —OEt | H | Br | 6-fluoropyridin-3-yl | O | — | 0 |
| I-03 | —OMe | (R)—Me | Br | pyridin-3-yl | O | 3-F | 1 |
| I-04 | —OMe | (R)—Me | CN | pyridin-3-yl | O | 3-F | 1 |
| I-05 | —OMe | (R)—Me | Br | 6-fluoropyridin-3-yl | O | 3-F | 1 |
| I-06 | —OMe | (R)—Me | CN | 6-fluoropyridin-3-yl | O | 3-F | 1 |
| I-07 | —OMe | H | Br | 6-fluoropyridin-3-yl | O | 3-F | 1 |
| I-08 | —OH | H | Br | 6-fluoropyridin-3-yl | O | 3-F | 1 |
| I-09 | —OMe | H | CN | 6-fluoropyridin-3-yl | O | 3-F | 1 |
| I-10 | —OH | (R)—Me | Br | 6-fluoropyridin-3-yl | O | 3-F | 1 |
| I-11 | —OMe | (R)—Me | Br | 6-fluoropyridin-3-yl | O | 5-F | 1 |
| I-12 | —OMe | H | Br | 6-fluoropyridin-3-yl | O | 5-F | 1 |
| I-13 | —OH | (R)—Me | Br | 5-fluoropyridin-3-yl | O | 5-F | 1 |
| I-14 | —OH | H | Br | 5-fluoropyridin-3-yl | O | 5-F | 1 |
| I-15 | —OMe | (R)—Me | Br | 5-fluoropyridin-3-yl | O | 3-F | 1 |
| I-16 | —OMe | H | Br | 5-fluoropyridin-3-yl | O | 3-F | 1 |
| I-17 | —OH | H | Br | 6-fluoropyridin-3-yl | O | 3-F | 1 |
| I-18 | —OH | (R)—Me | Br | 6-fluoropyridin-3-yl | O | 3-F | 1 |
| I-19 | —OMe | (R)—Me | Cl | 2-fluoropyridin-4-yl | O | — | 0 |
| I-20 | —OH | (R)—Me | Cl | pyrimidin-5-yl | O | — | 0 |
| I-21 | —OH | (R)—Me | Cl | 6-fluoropyridin-3-yl | O | — | 0 |
| I-22 | —OH | (R)—Me | Cl | 6-fluoropyridin-3-yl | O | — | 0 |
| I-23 | —OEt | H | Br | 6-fluoropyridin-3-yl | O | 3-Cl | 1 |
| I-24 | —OMe | (R)—Me | Br | 6-fluoropyridin-3-yl | O | 3-Cl | 1 |
| I-25 | —OH | (R)—Me | Br | 6-fluoropyridin-3-yl | O | 3-Cl | 1 |
| I-26 | —OH | H | Br | 6-fluoropyridin-3-yl | O | 3-Cl | 1 |
| I-27 | —OEt | (R)—Me | Br | 6-fluoropyridin-3-yl | O | 3-Cl | 1 |
| I-28 | —OMe | H | Br | 6-fluoropyridin-3-yl | O | 3-Cl | 1 |
| I-29 | —OCH₂CH₂OMe | H | Br | 6-fluoropyridin-3-yl | O | 3-F | 1 |
| I-30 | —OCH₂CN | H | Br | 6-fluoropyridin-3-yl | O | 3-F | 1 |
| I-31 | —NH-allyl | H | Br | 6-fluoropyridin-3-yl | O | 3-F | 1 |
| I-32 | —OEt | (R)—Me | Cl | 6-fluoropyridin-3-yl | O | 3-F | 1 |
| I-33 | —OEt | (R)—Me | Br | 6-fluoropyridin-3-yl | O | 3-F | 1 |
| I-34 | —OCH₂CH₂OCH₂CH₂OMe | (R)—Me | Br | 6-fluoropyridin-3-yl | O | 3-F | 1 |
| I-35 | —OCH₂CH₂OMe | (R)—Me | Br | 6-fluoropyridin-3-yl | O | 3-F | 1 |
| I-36 | —NHSO₂N(Me)₂ | H | Br | 6-fluoropyridin-3-yl | O | 3-F | 1 |
| I-37 | —OCH₂CH₂OCH₂CH₂OMe | H | Br | 6-fluoropyridin-3-yl | O | 3-F | 1 |
| I-38 | —OEt | H | Cl | 6-fluoropyridin-3-yl | O | 3-F | 1 |
| I-39 | —OEt | H | Br | 6-fluoropyridin-3-yl | O | 3-F | 1 |
| I-40 | —OEt | H | cyclopropyl | 6-fluoropyridin-3-yl | O | 3-F | 1 |
| I-41 | —OMe | (R)—Me | O | 6-fluoropyridin-3-yl | O | 3-F | 1 |
| I-42 | —OMe | H | cyclopropyl | 6-fluoropyridin-3-yl | O | 3-F | 1 |
| I-43 | —OH | H | cyclopropyl | 6-fluoropyridin-3-yl | O | 3-F | 1 |
| I-44 | —NHSO₂Me | H | Br | 6-fluoropyridin-3-yl | O | 3-F | 1 |
| I-45 | —O—N═C(CH₃)₂ | H | Br | 6-fluoropyridin-3-yl | O | 3-F | 1 |
| I-46 | —OCH₂-pyridin-2-yl | H | Br | 6-fluoropyridin-3-yl | O | 3-F | 1 |
| I-47 | 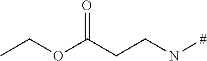 | H | Br | (6-methoxypyridin-3-yl) | O | 3-F | 1 |
| I-48 | 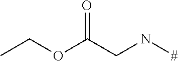 | H | Br | (6-methoxypyridin-3-yl) | O | 3-F | 1 |
| I-49 | —OMe | H | Br | 5-fluoropyridin-3-yl | O | 3-F | 1 |
| I-50 | —OH | (R)—Me | Br | 6-fluoropyridin-3-yl | O | 3-F | 1 |
| I-51 | —OEt | H | Br | 6-fluoropyridin-3-yl | O | 3-F | 1 |
| I-52 | —OCH₂-pyridin-2-yl | (R)—Me | Br | 6-fluoropyridin-3-yl | O | 3-F | 1 |
| I-53 | 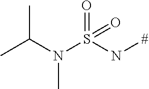 | H | Br | 6-fluoropyridin-3-yl | O | 3-F | 1 |
| I-54 | 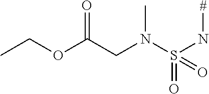 | H | Br | 6-fluoropyridin-3-yl | O | 3-F | 1 |
| I-55 | —OEt | H | I | 6-fluoropyridin-3-yl | O | 3-F | 1 |
| I-56 | —OEt | H | CF₃ | 6-fluoropyridin-3-yl | O | 3-F | 1 |
| I-57 | —OEt | H | NO₂ | 6-fluoropyridin-3-yl | O | 3-F | 1 |
| I-58 | —OEt | Me | Br | 6-fluoropyridin-3-yl | O | — | 0 |
| I-59 | 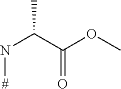 | H | Br | 2-fluoropyridin-4-yl | O | — | 0 |
| I-60 | 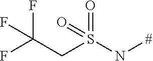 | H | Br | 6-fluoropyridin-3-yl | O | — | 0 |
| I-61 | 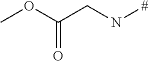 | H | Br | 6-fluoropyridin-3-yl | O | — | 0 |
| I-62 | 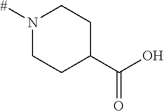 | H | Br | 6-fluoropyridin-3-yl | O | — | 0 |
| I-63 | 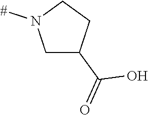 | H | Br | 5-fluoropyridin-3-yl | O | — | 0 |
| I-64 | 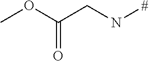 | H | Cl | 6-fluoropyridin-3-yl | O | — | 0 |
| I-65 | 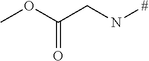 | H | Br | pyrimidin-5-yl | S | — | 0 |
| I-66 | —OH | H | Br | pyrimidin-5-yl | O | — | 0 |
| I-67 | prop-2-ynoxy | H | Cl | 5-fluoropyridin-3-yl | O | — | 0 |
| I-68 | —OEt | H | Br | 6-fluoropyridin-3-yl | O | — | 0 |
| I-69 | —OH | H | Br | 6-fluoropyridin-3-yl | S | — | 0 |
| I-70 | 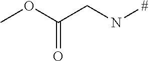 | H | cyclopropyl | 6-fluoropyridin-3-yl | O | — | 0 |
| I-71 | —OCH₂CH₂C(O)OMe | H | cyclopropyl | 5-fluoropyridin-3-yl | O | — | 0 |
| I-72 | —OH | H | cyclopropyl | 5-fluoropyridin-3-yl | O | — | 0 |
| I-73 | —OCH₂CH₂C(O)OMe | (R)—Me | Br | 5-fluoropyridin-3-yl | O | — | 0 |
| I-74 | 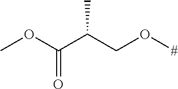 | H | Cl | 5-fluoropyridin-3-yl | O | — | 0 |
| I-75 | —OCH₂CH₂C(O)OMe | H | Cl | 5-fluoropyridin-3-yl | O | — | 0 |
| I-76 | 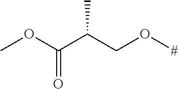 | H | Br | 6-fluoropyridin-3-yl | 0 | — | 0 |
| I-77 | —OCH₂CH₂C(O)OMe | H | Br | 5-fluoropyridin-3-yl | O | — | 0 |
| I-78 | —OEt | H | cyclopropyl | 6-fluoropyridin-3-yl | O | — | 0 |
| I-79 | —OH | (R)—Me | Br | 5-fluoropyridin-3-yl | O | — | 0 |
| I-80 | 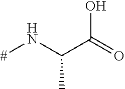 | H | Br | 5-fluoropyridin-3-yl | O | — | 0 |
| I-81 | —OH | H | Cl | 5-fluoropyridin-3-yl | O | — | 0 |
| I-82 | 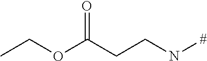 | H | Br | 5-fluoropyridin-3-yl | S | — | 0 |
| I-83 | —OMe | (R)—Me | Br | 5-fluoropyridin-3-yl | O | — | 0 |
| I-84 | —OH | H | Br | 5-fluoropyridin-3-yl | O | — | 0 |
| I-85 | —OEt | H | Cl | 6-fluoropyridin-3-yl | O | — | 0 |
| I-86 | —OEt | H | Br | 6-fluoropyridin-3-yl | O | — | 0 |
| I-87 | 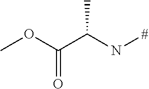 | H | Cl | 6-fluoropyridin-3-yl | O | — | 0 |
| I-88 | —OCH₂CH₂C(O)OMe | H | Cl | 6-fluoropyridin-3-yl | O | — | 0 |
| I-89 | 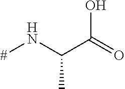 | H | Br | 6-fluoropyridin-3-yl | O | — | 0 |
| I-90 | —OCH₂-tetrahydrofuran-3-yl | H | Br | 6-fluoropyridin-3-yl | O | — | 0 |
| I-91 | —OEt | (R)—Me | Br | 6-fluoropyridin-3-yl | O | — | 0 |
| I-92 | 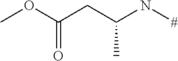 | H | Br | 6-fluoropyridin-3-yl | O | — | 0 |
| I-93 | —OCH₂-tetrahydrofuran-2-yl | H | Br | 6-fluoropyridin-3-yl | O | — | 0 |
| I-94 | —OH | Me | CF₃ | 6-fluoropyridin-3-yl | O | — | 0 |
| I-95 | —OCH₂CH₂SMe | H | Br | 6-fluoropyridin-3-yl | O | — | 0 |
| I-96 | —OCH₂CH₂OMe | H | Br | 6-fluoropyridin-3-yl | O | — | 0 |
| I-97 | 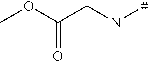 | H | Br | 6-fluoropyridin-3-yl | O | — | 0 |
| I-98 | —OH | H | Cl | 6-fluoropyridin-3-yl | O | — | 0 |
| I-99 | —OH | H | CF₃ | 6-fluoropyridin-3-yl | O | — | 0 |
| I-100 | —OEt | H | Cl | 6-fluoropyridin-3-yl | O | — | 0 |
| I-101 | —OEt | Me | I | 6-fluoropyridin-3-yl | O | — | 0 |
| I-102 | —OEt | Me | Br | 6-fluoropyridin-3-yl | O | — | 0 |
| I-103 | —OH | H | I | 6-fluoropyridin-3-yl | 0 | — | 0 |
| I-104 | —OEt | H | Br | 5-fluoropyridin-3-yl | SO₂ | — | 0 |
| I-105 | —OEt | H | CF₃ | 6-fluoropyridin-3-yl | 0 | — | 0 |
| I-106 | —OEt | H | Br | 5-fluoropyridin-3-yl | SO₂ | — | 0 |
| I-107 | —OEt | Me | CF₃ | 6-fluoropyridin-3-yl | O | — | 0 |
| I-108 | 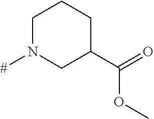 | H | Br | 6-fluoropyridin-3-yl | O | — | 0 |
| I-109 | 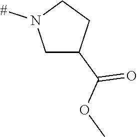 | H | Br | 6-fluoropyridin-3-yl | O | — | 0 |
| I-110 | 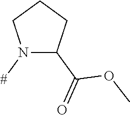 | H | Br | 6-fluoropyridin-3-yl | O | — | 0 |
| I-111 | 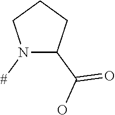 | H | Br | 6-fluoropyridin-3-yl | O | — | 0 |
| I-112 | 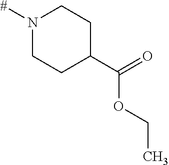 | H | Br | 6-fluoropyridin-3-yl | O | — | 0 |
| I-113 | —OEt | H | Br | 6-chloropyridin-3-yl | S | — | 0 |
| I-114 | 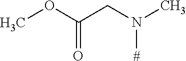 | H | Br | 6-fluoropyridin-3-yl | O | — | 0 |
| I-115 | —OEt | H | Br | pyrimidin-5-yl | S | — | 0 |
| I-116 | —OCH₂CH₂C(O)OMe | H | Br | 6-fluoropyridin-3-yl | O | — | 0 |
| I-117 | —NHCH₂CH₂C(O)OMe | H | Br | 6-fluoropyridin-3-yl | O | — | 0 |
| I-118 | 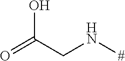 | H | Cl | 6-fluoropyridin-3-yl | O | — | 0 |
| I-119 | 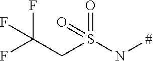 | H | Cl | 6-fluoropyridin-3-yl | O | — | 0 |
| I-120 | 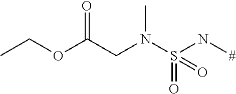 | H | Br | 6-fluoropyridin-3-yl | O | — | 0 |
| I-121 | —OEt | H | I | 6-fluoropyridin-3-yl | O | — | 0 |
| I-122 | —OH | H | Br | 2-fluoropyridin-4-yl | O | — | 0 |
| I-123 | —OEt | H | Br | 5-chloropyridin-3-yl | S | — | 0 |
| I-124 | —OEt | H | Br | 5-chloropyridin-3-yl | S | — | 0 |
| I-125 | —OEt | H | Br | 5-fluoropyridin-3-yl | S | — | 0 |
| I-126 | —OH | H | Br | 5-chloropyridin-3-yl | S | — | 0 |
| I-127 | —OH | (R)—Me | cyclopropyl | 2-fluoropyridin-4-yl | O | 3-F | 1 |
| I-128 | —OEt | H | cyclopropyl | 2-fluoropyridin-4-yl | O | 3-F | 1 |
| I-129 | —OMe | H | cyclopropyl | 2-fluoropyridin-4-yl | O | 3-F | 1 |
| I-130 | —OMe | H | Br | 2-fluoropyridin-4-yl | O | 3-F | 1 |
| I-131 | —OH | H | cyclopropyl | 2-fluoropyridin-4-yl | O | 3-F | 1 |
| I-132 | —OMe | (R)—Me | cyclopropyl | 2-fluoropyridin-4-yl | O | 3-F | 1 |
| I-133 | —OEt | (R)—Me | Br | 2-fluoropyridin-4-yl | O | 3-F | 1 |
| I-134 | —OMe | (R)—Me | Br | 2-fluoropyridin-4-yl | O | 3-F | 1 |
| I-135 | —OEt | (R)—Me | cyclopropyl | 2-fluoropyridin-4-yl | O | 3-F | 1 |
| I-136 | —OEt | H | Br | 2-fluoropyridin-4-yl | O | 3-F | 1 |
| I-137 | —OEt | H | I | 2-fluoropyridin-4-yl | O | 3-F | 1 |
| I-138 | —OMe | (R)—Me | I | 2-fluoropyridin-4-yl | O | 3-F | 1 |
| I-139 | —OH | H | Br | 2-fluoropyridin-4-yl | O | 3-F | 1 |
| I-140 | —OH | (R)—Me | Br | 2-fluoropyridin-4-yl | O | 3-F | 1 |
| I-141 | —OEt | H | CF₃ | 5-fluoropyridin-3-yl | O | 3-F | 1 |
| I-142 | —OCH₂CH₂C(O)OMe | (R)—Me | cyclopropyl | 5-fluoropyridin-3-yl | O | 3-F | 1 |
| I-143 | —OEt | H | prop-1-en-2-yl | 5-fluoropyridin-3-yl | O | 3-F,6-F | 2 |
| I-144 | —OMe | (R)—Me | cyclopropyl | 5-fluoropyridin-3-yl | O | 3-F | 1 |
| I-145 | —OEt | (R)—Me | Cl | 5-fluoropyridin-3-yl | O | 3-F | 1 |
| I-146 | —OMe | (R)—Me | Cl | 5-fluoropyridin-3-yl | O | 3-F | 1 |
| I-147 | —OEt | H | Cl | 5-fluoropyridin-3-yl | O | — | 0 |
| I-148 | —OH | H | cyclopropyl | 5-fluoropyridin-3-yl | O | 3-F,6-F | 2 |
| I-149 | —OEt | H | cyclopropyl | 5-fluoropyridin-3-yl | O | 3-F,6-F | 2 |
| I-150 | —OEt | H | Cl | 5-fluoropyridin-3-yl | SO | — | 0 |
| I-151 | —OMe | H | Br | 5-fluoropyridin-3-yl | S | — | 0 |
| I-152 | —OEt | H | I | 5-fluoropyridin-3-yl | S | — | 0 |
| I-153 | —OH | H | cyclopropyl | 5-fluoropyridin-3-yl | S | — | 0 |
| I-154 | —OMe | (R)-Me | Cl | 5-fluoropyridin-3-yl | O | 3-F,6--F | 2 |
| I-155 | —OH | (R)-Me | Br | 5-fluoropyridin-3-yl | O | 3-F,6-F | 2 |
| I-156 | —OEt | H | cyclopropyl | 5-fluoropyridin-3-yl | S | — | 0 |
| I-157 | —OCH₂CH₂OMe | H | Br | 5-fluoropyridin-3-yl | O | 3-CF3 | 1 |
| I-158 | —OCH₂CH₂C(O)OMe | (R)-Me | cyclopropyl | 5-fluoropyridin-3-yl | O | 3-F,6-F | 2 |
| I-159 | —OH | (R)-Me | Cl | 5-fluoropyridin-3-yl | 0 | 3-F,6-F | 2 |
| I-160 | —OCH₂CH₂C(O)OMe | H | Br | 5-fluoropyridin-3-yl | O | 3-CF3 | 1 |
| I-161 | —OEt | H | Br | 5-fluoropyridin-3-yl | O | 5-Br-6- | 2 |
| | | | | | | Me | |
| I-162 | —OCH₂-tetrahydrofuran-3-yl | H | Br | 5-fluoropyridin-3-yl | O | 3-CF₃ | 1 |
| I-163 | —OEt | H | Cl | 5-fluoropyridin-3-yl | O | 3-F,6-F | 2 |
| I-164 | —OMe | H | cyclopropyl | 5-fluoropyridin-3-yl | O | 3-F,6-F | 2 |
| I-165 | —OH | (R)—Me | Cl | 5-fluoropyridin-3-yl | O | 3-F | 1 |
| I-166 | —OH | (R)—Me | cyclopropyl | 5-fluoropyridin-3-yl | O | 3-F | 1 |
| I-167 | —OMe | H | cyclopropyl | 5-fluoropyridin-3-yl | O | 3-F | 1 |
| I-168 | —OEt | H | Cl | 5-fluoropyridin-3-yl | C | 3-F | 1 |
| I-169 | —OH | H | Cl | 5-fluoropyridin-3-yl | O | 3-F | 1 |
| I-170 | —OMe | H | Cl | 5-fluoropyridin-3-yl | O | 3-F | 1 |
| I-171 | —OMe | (R)—Me | cyclopropyl | 5-fluoropyridin-3-yl | O | 3-F,6-F | 2 |
| I-172 | —OEt | (R)—Me | cyclopropyl | 5-fluoropyridin-3-yl | O | 3-F | 1 |
| I-173 | —OEt | H | F | 5-fluoropyridin-3-yl | O | — | 0 |
| I-174 | —OEt | H | cyclopropyl | 5-fluoropyridin-3-yl | O | 3-F | 1 |
| I-175 | —OH | H | cyclopropyl | 5-fluoropyridin-3-yl | O | 3-F | 1 |
| I-176 | —OCH(CH₃)C(O)OMe | (R)—Me | Br | 5-fluoropyridin-3-yl | O | — | 0 |
| I-177 | —OEt | (R)—Me | cyclopropyl | 5-fluoropyridin-3-yl | O | 3-F,6-F | 2 |
| I-178 | —OMe | (R)—Me | CF₃ | 5-fluoropyridin-3-yl | O | 3-F | 1 |
| I-179 | —OMe | H | Br | 5-fluoropyridin-3-yl | O | 3-Me | 1 |
| I-180 | —OH | (R)—Me | cyclopropyl | 5-fluoropyridin-3-yl | O | 3-F,6-F | 2 |
| I-181 | —OEt | H | Cl | 5-fluoropyridin-3-yl | O | 3-Me | 1 |
| I-182 | —OH | H | Cl | 5-fluoropyridin-3-yl | O | 3-Me | 1 |
| I-183 | —OEt | H | Cl | 5-fluoropyridin-3-yl | S | — | 0 |
| I-184 | —OEt | (R)—Me | Cl | 5-fluoropyridin-3-yl | O | — | 0 |
| I-185 | —NHCH₂C(O)OMe | (R)—Me | Cl | 5-fluoropyridin-3-yl | O | — | 0 |
| I-186 | —OH | H | I | 5-fluoropyridin-3-yl | O | 3-CF₃ | 1 |
| I-187 | —NHCH₂COOMe | (R)—Me | Br | 5-fluoropyridin-3-yl | O | — | 0 |
| I-188 | —OCH₂CH(CH₃)C(O)OMe | (R)—Me | Br | 5-fluoropyridin-3-yl | O | — | 0 |
| I-189 | —OMe | (R)—Me | Cl | 5-fluoropyridin-3-yl | O | — | 0 |
| I-190 | —NHCH(CH₃)COOMe | (R)—Me | Br | 5-fluoropyridin-3-yl | O | — | 0 |
| I-191 | —OMe | (R)—Me | Br | 5-fluoropyridin-3-yl | O | 3-F,6-F | 2 |
| I-192 | —OCH₂CH—(R)—(CH₃)C(O)OMe | H | Cl | 5-fluoropyridin-3-yl | O | — | 0 |
| I-193 | —OEt | H | Br | 5-fluoropyridin-3-yl | O | 3-F,6-F | 2 |
| I-194 | —OCH₂CH—(S)—(CH₃)C(O)OMe | H | Br | 5-fluoropyridin-3-yl | O | — | 0 |
| I-195 | —OCH₂CH—(R)—(CH₃)C(O)OMe | H | Br | 5-fluoropyridin-3-yl | O | — | 0 |
| I-196 | —OCH₂CH₂COOMe | (R)—Me | Cl | 5-fluoropyridin-3-yl | O | — | |
| I-197 | —OMe | (R)—Me | I | 5-fluoropyridin-3-yl | O | 3-F | 1 |
| I-198 | —OCH₂CH(CH₃)C(O)OMe | (R)—Me | Cl | 5-fluoropyridin-3-yl | O | — | 0 |
| I-199 | —NHCH(CH₃)COOMe | (R)—Me | Cl | 5-fluoropyridin-3-yl | O | — | 0 |
| I-200 | —NHSO₂phenyl | H | Br | 5-fluoropyridin-3-yl | S | — | 0 |
| I-201 | —NH-cyclopropyl | H | Br | 5-fluoropyridin-3-yl | S | — | 0 |
| I-202 | —OEt | H | Br | 5-fluoropyridin-3-yl | O | 3-CF₃ | 1 |
| I-203 | —OEt | (R)—Me | vinyl | 5-fluoropyridin-3-yl | O | 3-F,6-F | 2 |
| I-204 | —OH | H | Br | 5-fluoropyridin-3-yl | O | 3-Me | 1 |
| I-205 | —NHCH₂C(O)OMe | H | Br | 5-fluoropyridin-3-yl | O | 3-CF₃ | 1 |
| I-206 | —OMe | H | Br | 5-fluoropyridin-3-yl | O | 3-CF₃ | 1 |
| I-207 | —OH | H | Br | 5-fluoropyridin-3-yl | O | 3-F,6-F | 2 |
| I-208 | —OEt | (R)—Me | Cl | 5-fluoropyridin-3-yl | O | 3-F,6-F | 2 |
| I-209 | —OEt | (R)—Me | prop-1-en-2-yl | 5-fluoropyridin-3-yl | O | 3-F,6-F | 2 |
| I-210 | —OMe | H | Br | 5-fluoropyridin-3-yl | O | 3-F,6-F | 2 |
| I-211 | —OEt | H | Br | 5-fluoropyridin-3-yl | O | 3-Me | 1 |
| I-212 | —OCH₂CH₂OMe | H | Br | 5-fluoropyridin-3-yl | O | 3-Me | 1 |
| I-213 | —OMe | (R)—Me | 2,2- | 5-fluoropyridin-3-yl | O | 3-F,6-F | 2 |
| | | | difluorocyclo- | | | | |
| | | | propyl | | | | |
| I-214 | —OH | H | prop-1-en-2-yl | 5-fluoropyridin-3-yl | O | 3-F,6-F | 2 |
| I-215 | —OMe | H | prop-1-en-2-yl | 5-fluoropyridin-3-yl | O | 3-F,6-F | 2 |
| I-216 | —OMe | H | Cl | 5-fluoropyridin-3-yl | O | 3-F,6-F | 2 |
| I-217 | —OH | H | Cl | 5-fluoropyridin-3-yl | S | — | 0 |
| I-218 | —OH | (R)—Me | Cl | 5-fluoropyridin-3-yl | O | — | 0 |
| I-219 | —OH | (R)—Me | vinyl | 5-fluoropyridin-3-yl | O | 3-F,6-F | 2 |
| I-220 | —OEt | H | cyclobutyl | 5-fluoropyridin-3-yl | O | — | 0 |
| I-221 | OCH₂CH—(S)—(CH₃)C(O)OMe | H | Cl | 5-fluoropyridin-3-yl | O | — | 0 |
| I-222 | —OMe | (R)—Me | prop-1-en-2-yl | 5-fluoropyridin-3-yl | O | 3-F,6-F | 2 |
| I-223 | —OMe | H | I | 5-fluoropyridin-3-yl | O | 3-CF3 | 1 |
| I-224 | —OEt | H | vinyl | 5-fluoropyridin-3-yl | O | 3-F,6-F | 2 |
| I-225 | —OEt | H | I | 5-fluoropyridin-3-yl | O | 3-CF3 | 1 |
| I-226 | —OMe | (R)—Me | vinyl | 5-fluoropyridin-3-yl | O | 3-F,6-F | 2 |
| I-227 | —OEt | (R)—Me | Br | 5-fluoropyridin-3-yl | O | 3-F | 1 |
| I-228 | —OH | (R)—Me | prop-1-en-2-yl | 5-fluoropyridin-3-yl | O | 3-F,6-F | 2 |
| I-229 | —OH | H | I | 5-fluoropyridin-3-yl | S | — | |
| I-230 | —OH | H | Br | 5-fluoropyridin-3-yl | O | 3-CF3 | 1 |
| I-231 | —OH | H | Cl | 5-fluoropyridin-3-yl | O | 3-F,6-F | 2 |
| I-232 | —OMe | (R)—Me | vinyl | 5-fluoropyridin-3-yl | O | 3-F | 1 |
| I-233 | —OMe | H | Cl | 5-fluoropyridin-3-yl | O | 3-Me | 1 |
| I-234 | —OEt | H | vinyl | 5-fluoropyridin-3-yl | O | 3-F | 1 |
| I-235 | —OH | H | Br | 6-chloropyridin-3-yl | O | — | 0 |
| I-236 | —OH | H | Cl | 6-chloropyridin-3-yl | O | — | 0 |
| I-237 | —OMe | (R)—Me | Cl | 6-chloropyridin-3-yl | O | — | 0 |
| I-238 | —OH | (R)—Me | Cl | 6-chloropyridin-3-yl | O | — | 0 |
| I-239 | —OEt | H | Br | 6-chloropyridin-3-yl | O | — | 0 |
| I-240 | —OH | (R)—Me | Br | 6-chloropyridin-3-yl | O | — | 0 |
| I-241 | —OEt | H | Cl | 6-chloropyridin-3-yl | O | — | 0 |
| I-242 | —OMe | (R)—Me | Br | 6-chloropyridin-3-yl | O | — | 0 |
| I-243 | —OCH₂CH₂C(O)OMe | H | Cl | 6-chloropyridin-3-yl | O | — | 0 |
| I-244 | —OCH₂CH₂C(O)OMe | (R)—Me | Br | 6-chloropyridin-3-yl | O | — | 0 |
| I-245 | —OCH₂CH₂C(O)OMe | (R)—Me | Cl | 6-chloropyridin-3-yl | O | — | 0 |
| I-246 | —OCH₂CH₂C(O)OMe | H | Br | 6-chloropyridin-3-yl | O | — | 0 |
| I-247 | —OCH₂CH₂C(O)OMe | H | Br | 6-fluoropyridin-3-yl | O | — | 0 |
| I-248 | —OCH₂CH₂C(O)OMe | H | 2,2 | 6-fluoropyridin-3-yl | O | — | 0 |
| | | | difluorocyclo- | | | | |
| | | | propyl | | | | |
| I-249 | —OH | H | 2,2 | 6-fluoropyridin-3-yl | O | — | 0 |
| | | | difluorocyclo- | | | | |
| | | | propyl | | | | |
| I-250 | —NHCH₂CH₂COOEt | H | cyclopropyl | 6-fluoropyridin-3-yl | O | 3-F | 1 |
| I-251 | OCH₂-cyclopentyl | H | cyclopropyl | 6-fluoropyridin-3-yl | O | 3-F | 1 |
| I-252 | —OMe | (R)—Me | Br | 6-fluoropyridin-3-yl | O | — | 0 |
| I-253 | —OCH₂-3-pyridyl | H | cyclopropyl | 6-fluoropyridin-3-yl | O | 3-F | 1 |
| I-254 | —NHCH₂C(O)OMe | H | cyclopropyl | 6-fluoropyridin-3-yl | O | 3-F | 1 |
| I-255 | —OH | H | cyclopropyl | 6-fluoropyridin-3-yl | O | 3-CF₃ | 1 |
| I-256 | —OCH₂CH₂C(O)OMe | (R)—Me | Cl | 6-fluoropyridin-3-yl | O | 3-CF₃ | 1 |
| I-257 | —OCH(CH₃)C(O)OMe | (R)—Me | Cl | 6-fluoropyridin-3-yl | O | 3-CF₃ | 1 |
| I-258 | —OEt | H | cyclopropyl | 6-fluoropyridin-3-yl | O | 3-CF₃ | 1 |
| I-259 | —NHCH₂C(O)OEt | H | cyclopropyl | 6-fluoropyridin-3-yl | O | — | 0 |
| I-260 | —OH | (R)—Me | Br | 6-fluoropyridin-3-yl | O | — | 0 |
| I-261 | —OCH₂CH₂C(O)OMe | H | 2,2- | 6-fluoropyridin-3-yl | O | — | 0 |
| | | | difluorocyclo- | | | | |
| | | | propyl | | | | |
| I-263 | —OH | H | 2,2 | 6-fluoropyridin-3-yl | O | 3-F | 1 |
| | | | difluorocyclo- | | | | |
| | | | propyl | | | | |
| I-264 | —OCH₂-oxetan-3-yl | H | cyclopropyl | 6-fluoropyridin-3-yl | O | 3-F | 1 |
| I-265 | —OEt | H | prop-1-en-2-yl | 6-fluoropyridin-3-yl | O | 3-F | 1 |
| I-266 | —OH | H | Cl | 6-fluoropyridin-3-yl | O | — | 0 |
| I-267 | —OCH₂C(CH₃)C(O)OMe | (R)—Me | Cl | 6-fluoropyridin-3-yl | O | — | 0 |
| I-268 | —OCH2CH(CH3)C(O)OMe | H | 2,2- | 6-fluoropyridin-3-yl | 0 | 3-CF₃ | 1 |
| | | | difluorocyclo- | | | | |
| | | | propyl | | | | |
| I-269 | —OH | (R)—Me | Cl | 6-fluoropyridin-3-yl | O | 3-CF₃ | 1 |
| I-270 | —OCH₂CH₂OCF₃ | H | cyclopropyl | 6-fluoropyridin-3-yl | O | 3-F | 1 |
| I-272 | —OCH₂CH₂C(O)OMe | H | Cl | 6-fluoropyridin-3-yl | O | 3-F | 1 |
| I-273 | —OEt | H | vinyl | 6-fluoropyridin-3-yl | O | — | |
| I-274 | —OCH₂-tetrahydrofuran-2-yl | H | cyclopropyl | 6-fluoropyridin-3-yl | O | 3-F | 1 |
| I-275 | —OCH₂CH—(R)—(CH₃)C(O)OMe | (R)—Me | Cl | 6-fluoropyridin-3-yl | O | — | 0 |
| I-276 | —OCH₂CH₂C(O)OMe | H | Cl | 6-fluoropyridin-3-yl | O | 6-Me | 1 |
| I-277 | —OCH₂CH₂OMe | H | Br | 6-fluoropyridin-3-yl | O | 6-Me | 1 |
| I-278 | —OCH₂CH₂C(O)OMe | H | Br | 6-fluoropyridin-3-yl | O | 6-Me | 1 |
| I-279 | —OCH₂CH₂OMe | H | Cl | 6-fluoropyridin-3-yl | O | 6-Me | 1 |
| I-280 | —OCH₂-4-pyridinyl | H | cyclopropyl | 6-fluoropyridin-3-yl | O | 3-F | 1 |
| I-281 | —OMe | (R)—Me | Cl | 6-fluoropyridin-3-yl | O | 3-CF₃ | 1 |
| I-282 | —OCH₂-2-pyridinyl | H | cyclopropyl | 6-fluoropyridin-3-yl | O | 3-F | 1 |
| I-283 | —OCH₂CH(CH₃)C(O)OMe | (R)—Me | Cl | 6-fluoropyridin-3-yl | O | 3-CF₃ | 1 |
| I-285 | —OCH₂CH(CH₃)C(O)OMe | (R)—Me | Br | 6-fluoropyridin-3-yl | O | — | 0 |
| I-286 | —NHCH(CH₃)C(O)OMe | (R)—Me | Br | 6-fluoropyridin-3-yl | O | — | 0 |
| I-287 | —OCH₂Ph | H | cyclopropyl | 6-fluoropyridin-3-yl | O | 3-F | 1 |
| I-288 | —OCH(CH₃)C(O)OMe | (R)—Me | Br | 6-fluoropyridin-3-yl | O | — | 0 |
| I-289 | —OCH₂-tetrahydrofuran-3-yl | H | cyclopropyl | 6-fluoropyridin-3-yl | O | 3-F | 1 |
| I-290 | —OCH₂CH₂C(O)OMe | (R)—Me | Br | 6-fluoropyridin-3-yl | O | — | 0 |
| I-291 | —OCH₂CH(CH₃)C(O)OMe | (R)—Me | Cl | 6-fluoropyridin-3-yl | O | — | 0 |
| I-293 | —OCH₂CH₂C(O)OMe | Me | CF3 | 6-fluoropyridin-3-yl | O | — | 0 |
| I-294 | —OCH₂CN | H | cyclopropyl | 6-fluoropyridin-3-yl | O | 3-F | 1 |
| I-295 | —OH | H | prop-1-en-2-yl | 6-fluoropyridin-3-yl | O | 3-F | 1 |
| I-296 | —OH | (R)—Me | cyclopropyl | 6-fluoropyridin-3-yl | O | 3-F,6-F | 2 |
| I-297 | —NHCH(CH₃)—CH═CH—C(O)OMe | H | Br | 6-fluoropyridin-3-yl | O | — | 0 |
| I-298 | —OMe | (R)—Me | cyclopropyl | 6-fluoropyridin-3-yl | O | 3-F,6-F | 2 |
| I-299 | —OCH(CH₃)C(O)OMe | (R)—Me | Cl | 6-fluoropyridin-3-yl | O | — | 0 |
| I-300 | —OMe | H | cyclopropyl | 6-fluoropyridin-3-yl | O | 3-F,6-F | 2 |
| I-301 | —NHCH₂CH₂C(O)OMe | H | cyclopropyl | 6-fluoropyridin-3-yl | O | 3-F | 1 |
| I-302 | —OH | H | cyclopropyl | 6-fluoropyridin-3-yl | O | 3-F,6-F | 2 |
| I-303 | —NHCH(CH₃)C(O)OEt | H | cyclopropyl | 6-fluoropyridin-3-yl | O | 3-F | 1 |
| I-304 | —OMe | H | Cl | 6-fluoropyridin-3-yl | O | 3-F | 1 |
| I-305 | —OEt | H | F | 6-fluoropyridin-3-yl | O | — | 0 |
| I-306 | —OH | (R)—Me | F | 6-fluoropyridin-3-yl | O | 3-F | 1 |
| I-307 | —OMe | (R)—Me | cyclopropyl | 6-fluoropyridin-3-yl | O | 3-F | 1 |
| I-308 | —OEt | (R)—Me | CF₃ | 6-fluoropyridin-3-yl | O | 3-F | 1 |
| I-309 | —OCH₂CH₂C(O)OMe | H | F | 6-fluoropyridin-3-yl | O | — | 0 |
| I-310 | —OH | H | Br | 6-fluoropyridin-3-yl | O | 3-CF₃ | 1 |
| I-311 | —OH | (R)—Me | Cl | 6-fluoropyridin-3-yl | O | 3-F,6-F | 2 |
| I-312 | —OEt | H | Br | 6-fluoropyridin-3-yl | O | 6-Me | 1 |
| I-313 | —OCH₂CH₂SO₂Me | H | Br | 6-fluoropyridin-3-yl | O | — | 0 |
| I-314 | —OCH₂CH₂OMe | H | cyclopropyl | 6-fluoropyridin-3-yl | O | 3-F | 1 |
| I-315 | —OEt | H | Cl | 6-fluoropyridin-3-yl | O | 3-F,6-F | 2 |
| I-316 | —OEt | H | Cl | 6-fluoropyridin-3-yl | O | 3-Me | 1 |
| I-317 | —OH | (R)—Me | CF₃ | 6-fluoropyridin-3-yl | O | 3-F | 1 |
| I-318 | —OCH₂-cyclopropyl | H | cyclopropyl | 6-fluoropyridin-3-yl | O | 3-F | 1 |
| I-319 | —OMe | (R)—Me | Br | 6-fluoropyridin-3-yl | O | 3-F,6-F | |
| I-320 | —OEt | (R)—Me | F | 6-fluoropyridin-3-yl | O | 3-F | 1 |
| I-321 | —OEt | H | Br | 6-fluoropyridin-3-yl | O | 3-Me | 1 |
| I-322 | 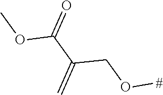 | (R)—Me | Cl | 6-fluoropyridin-3-yl | O | — | 0 |
| I-323 | —OH | H | Cl | 6-fluoropyridin-3-yl | O | 3-Me | 1 |
| I-324 | —OH | H | Br | 6-fluoropyridin-3-yl | O | 3-Me | 1 |
| I-325 | —OEt | H | Cl | 6-fluoropyridin-3-yl | O | 3-CF₃ | 1 |
| I-326 | —OCH₂CF₃ | H | cyclopropyl | 6-fluoropyridin-3-yl | O | 3-F | 1 |
| I-327 | —OH | H | Cl | 6-fluoropyridin-3-yl | O | 3-CF₃ | 1 |
| I-328 | —OiPr | H | cyclopropyl | 6-fluoropyridin-3-yl | O | 3-F | 1 |
| I-329 | —OH | H | Cl | 6-fluoropyridin-3-yl | O | 3-OMe | 1 |
| I-330 | —OEt | H | Br | 6-fluoropyridin-3-yl | O | 3-F,6-F | 2 |
| I-331 | —OMe | (R)—Me | CF₃ | (6-fluoropyridin-3-yl) | O | 3-F | 1 |
| I-332 | —OMe | (R)—Me | Cl | 6-fluoropyridin-3-yl | O | 3-F,6-F | 2 |
| I-333 | —OH | (R)—Me | Br | 6-fluoropyridin-3-yl | O | 3-F,6-F | 2 |
| I-334 | —OH | H | Br | 6-fluoropyridin-3-yl | O | 3-F,6-F | 2 |
| I-335 | —OEt | H | Br | 6-fluoropyridin-3-yl | O | 3-CF3 | 1 |
| I-336 | —OEt | (R)—Me | Br | 6-fluoropyridin-3-yl | O | 3-F,6-F | 2 |
| I-337 | —OH | H | Cl | 6-fluoropyridin-3-yl | O | 3-F,6-F | 2 |
| I-338 | —NHCH₂COOH | (R)—Me | Cl | 6-fluoropyridin-3-yl | O | — | 0 |
| I-339 | —NHCH₂COOMe | (R)—Me | Cl | 6-fluoropyridin-3-yl | O | — | 0 |
| I-340 | —OCH₂CH₂COOMe | (R)—Me | Cl | 6-fluoropyridin-3-yl | O | — | 0 |
| I-341 | —OMe | (R)—Me | I | 6-fluoropyridin-3-yl | O | 3-F | 1 |
| I-342 | —OH | (R)—Me | cyclopropyl | 6-fluoropyridin-3-yl | O | 3-F | 1 |
| I-343 | —OH | (R)—Me | I | 6-fluoropyridin-3-yl | O | 3-F | 1 |
| I-344 | —OPr | H | cyclopropyl | 6-fluoropyridin-3-yl | O | 3-F | 1 |
| I-345 | —NHCH(CH₃)—CH═CHC(O)OMe | H | Br | 6-fluoropyridin-3-yl | O | — | 0 |
| I-346 | —OMe | H | Cl | 6-fluoropyridin-3-yl | O | 3-OMe | 1 |
| I-347 | —OH | isopropyl | Br | 6-fluoropyridin-3-yl | O | — | 0 |
| I-348 | —OEt | H | cyclopropyl | 6-fluoropyridin-3-yl | O | 3-F,6-F | 2 |
| I-349 | —OEt | isopropyl | Br | 6-fluoropyridin-3-yl | O | — | 0 |
| I-350 | —NHCH(CH₃)C(O)OH | (R)—Me | Cl | 6-fluoropyridin-3-yl | O | — | 0 |
| I-351 | —OH | H | Cl | 6-fluoropyridin-3-yl | O | 6-Me | 1 |
| I-352 | —OEt | H | Cl | 6-fluoropyridin-3-yl | O | 6-Me | 1 |
| I-353 | —OMe | H | 3-methylbut-2- | 6-fluoropyridin-3-yl | O | 3-F | 1 |
| | | | en-2-yl | | | | |
| I-354 | —OEt | H | fluoro | 6-fluoropyridin-3-yl | O | 3-F | 1 |
| I-355 | —OMe | H | 2,2- | 6-fluoropyridin-3-yl | O | 3-F | 1 |
| | | | difluorocyclo- | | | | |
| | | | propy | | | | |
| I-356 | —OCH₂CH₂C(O)OMe | H | cyclopropyl | 6-fluoropyridin-3-yl | O | 3-CF₃ | 1 |
| I-357 | —NHCH₂C(O)OMe | H | Br | 6-fluoropyridin-3-yl | O | 6-Me | 1 |
| I-358 | —NHCH(CH₃)C(O)OMe | (R)—Me | Cl | 6-fluoropyridin-3-yl | O | — | 0 |
| I-359 | —OEt | H | nitro | 6-fluoropyridin-3-yl | O | — | 0 |
| I-360 | prop-2-ynoxy | H | Br | 6-fluoropyridin-3-yl | O | — | 0 |
| I-361 | —OCH₂CH(CH₃)C(O)OMe | H | cyclopropyl | 6-fluoropyridin-3-yl | O | 3-CF₃ | 1 |
| I-362 | —OCH₂CH₂OMe | H | Cl | 6-fluoropyridin-3-yl | O | 3-CF₃ | 1 |
| I-363 | —OH | H | Br | 6-fluoropyridin-3-yl | O | 3-OMe | 1 |
| I-365 | —OCH₂CH₂C(O)OMe | H | 2,2- | 6-fluoropyridin-3-yl | O | 3-F | 1 |
| | | | difluorocyclo- | | | | |
| | | | propyl | | | | |
| I-366 | —ON═C(CH₃)₂ | H | cyclopropyl | 6-fluoropyridin-3-yl | O | 3-F | 1 |
| I-367 | —OMe | H | prop-1-en-2-yl | 6-fluoropyridin-3-yl | O | 3-F | 1 |
| I-368 | —OEt | H | vinyl | 6-fluoropyridin-3-yl | O | 3-F | 1 |
| I-369 | —OMe | H | Br | 6-fluoropyridin-3-yl | O | 3-F,6-F | 2 |
| I-370 | 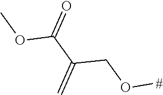 | H | Br | 6-fluoropyridin-3-yl | O | — | 0 |
| I-371 | —OEt | H | Br | 6-fluoropyridin-3-yl | O | 3-OMe | 1 |
| I-372 | —OEt | H | cyclobutyl | 6-fluoropyridin-3-yl | O | — | 0 |
| I-373 | —OH | H | F | 6-fluoropyridin-3-yl | O | — | 0 |
| I-374 | —ON═C(CH₃)₂ | (R)—Me | Br | 6-fluoropyridin-3-yl | O | 3-F | 1 |
| I-375 | —OEt | (R)—Me | cyclopropyl | 6-fluoropyridin-3-yl | O | 3-F | 1 |
| I-376 | —OH | H | Br | 6-fluoropyridin-3-yl | O | 6-Me | 1 |
| I-377 | —NHCH₂C(O)OMe | H | F | 6-fluoropyridin-3-yl | O | H | |
| I-378 | —OCH₂CH₂OMe | H | Br | 6-fluoropyridin-3-yl | O | 3-CF₃ | 1 |
| I-380 | —ON═cyclopentyl | H | cyclopropyl | 6-fluoropyridin-3-yl | O | 3-F | 1 |
| I-381 | —OCH₂CH₂C(O)OMe | H | Br | 6-fluoropyridin-3-yl | O | 3-CF₃ | 1 |
| I-382 | —OCH₂CH(CH₃)C(O)OMe | H | cyclopropyl | 6-fluoropyridin-3-yl | O | 3-F | 1 |
| I-383 | —OCH₂CH₂C(O)OMe | H | cyclopropyl | 6-fluoropyridin-3-yl | O | 3-F | 1 |
| I-384 | —OEt | H | 2,2- | 6-fluoropyridin-3-yl | O | 3-F | 1 |
| | | | difluorocyclo- | | | | |
| | | | propyl | | | | |
| I-385 | —OH | H | vinyl | 6-fluoropyridin-3-yl | O | 3-F | 1 |
| I-386 | —OEt | H | I | 6-fluoropyridin-3-yl | O | 3-OMe | 1 |
| I-387 | —OEt | H | 2,2 | 6-fluoropyridin-3-yl | O | — | 0 |
| | | | difluorocyclo- | | | | |
| | | | propyl | | | | |
| I-388 | —OEt | (R)—Me | Br | 5-fluoropyridin-3-yl | O | 3-F,6-F | 2 |
| I-389 | —OMe | (R)—Me | 2,2- | 5-fluoropyridin-3-yl | O | 3-F | 1 |
| | | | difluorocyclo- | | | | |
| | | | propyl | | | | |
| I-390 | —OH | (R)—Me | vinyl | 5-fluoropyridin-3-yl | O | 3-F | 1 |
| I-391 | —OH | H | vinyl | 5-fluoropyridin-3-yl | O | 3-F | 1 |
| I-392 | —OEt | (R)—Me | vinyl | 5-fluoropyridin-3-yl | O | 3-F | 1 |
| I-393 | —OMe | H | vinyl | 5-fluoropyridin-3-yl | O | 3-F | 1 |
| I-394 | —OEt | H | 2,2- | 5-fluoropyridin-3-yl | O | 3-F | 1 |
| | | | difluorocyclo- | | | | |
| | | | propyl | | | | |
| I-395 | —OMe | (R)—Me | I | 6-fluoropyridin-3-yl | O | 3-Me | 1 |
| I-396 | —OEt | H | I | 6-fluoropyridin-3-yl | O | 3-Me | 1 |
| I-397 | —OH | H | 2,2 | 5-fluoropyridin-3-yl | O | 3-F | 1 |
| | | | difluorocyclo- | | | | |
| | | | propyl | | | | |
| I-398 | —OMe | H | 2,2- | 5-fluoropyridin-3-yl | O | 3-F | 1 |
| | | | difluorocyclo- | | | | |
| | | | propyl | | | | |
| I-399 | —OH | H | vinyl | 5-fluoropyridin-3-yl | O | 3-F,6-F | 2 |

TABLE II
Q = Q2 in formula (I)

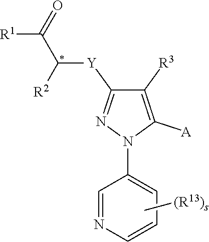

The linkage of R1 is indicated either by “-” or “#”.

| Example | | | | | | | |
|---|---|---|---|---|---|---|---|
| number | R¹ | R² | R³ | A | Y | R¹² | s |
| II-01 | —OEt | H | Br | 6-fluoropyridin-3-yl | O | 2-F | 1 |
| II-03 | —OH | H | Br | 6-fluoropyridin-3-yl | O | 2-F | 1 |
| II-04 | —OH | (R)—Me | Br | 6-fluoropyridin-3-yl | O | 2-F | 1 |
| II-05 | —OEt | (R)—Me | Br | 6-fluoropyridin-3-yl | O | 2-F | 1 |
| II-06 | CF₃CH₂SO₂NH- | H | Br | 6-fluoropyridin-3-yl | O | 2-F | 1 |
| II-07 | Me₂NSO₂NH- | H | Br | 6-fluoropyridin-3-yl | O | 2-F | 1 |
| II-08 | MeO(O)CCH₂NH- | H | Br | 6-fluoropyridin-3-yl | O | 2-F | 1 |
| II-09 | —OEt | H | Br | 6-fluoropyridin-3-yl | O | — | 0 |
| II-10 | —OH | H | Br | 6-fluoropyridin-3-yl | O | — | 0 |
| II-11 | —OH | (R)—Me | Br | 6-fluoropyridin-3-yl | O | — | 0 |
| II-12 | —OEt | (R)—Me | Br | 6-fluoropyridin-3-yl | O | — | 0 |
| II-13 | CF₃CH₂SO₂NH- | H | Br | 6-fluoropyridin-3-yl | O | — | 0 |
| II-14 | Me₂NSO₂NH- | H | Br | 6-fluoropyridin-3-yl | O | — | 0 |
| II-15 | MeO(O)CCH₂NH- | H | Br | 6-fluoropyridin-3-yl | O | 2-F | 1 |
| II-16 | —OEt | H | Br | pyrimidin-5-yl | O | 2-F | 1 |
| II-17 | —OH | H | Br | pyrimidin-5-yl | O | 2-F | 1 |
| II-18 | —OH | (R)—Me | Br | pyrimidin-5-yl | O | 2-F | 1 |
| II-19 | —OEt | (R)—Me | Br | pyrimidin-5-yl | O | 2-F | 1 |
| II-20 | CF₃CH₂SO₂NH— | H | Br | pyrimidin-5-yl | O | 2-F | 1 |
| II-21 | Me₂NSO₂NH— | H | Br | pyrimidin-5-yl | O | 2-F | 1 |
| II-22 | MeO(O)CCH₂NH— | H | Br | pyrimidin-5-yl | O | 2-F | 1 |
| II-23 | —OEt | H | Br | pyrimidin-5-yl | O | — | 0 |
| II-24 | —OH | H | Br | pyrimidin-5-yl | O | — | 0 |
| II-25 | —OH | (R)—Me | Br | pyrimidin-5-yl | O | — | 0 |
| II-26 | —OEt | (R)—Me | Br | pyrimidin-5-yl | O | — | 0 |
| II-27 | CF₃CH₂SO₂NH— | H | Br | pyrimidin-5-yl | O | — | 0 |
| II-28 | Me₂NSO₂NH— | H | Br | pyrimidin-5-yl | O | — | 0 |
| II-29 | MeO(O)CCH₂NH— | H | Br | pyridimin-5-yl | O | — | 0 |
| II-30 | —OH | (R)—Me | CF₃ | 6-fluoropyridin-3-yl | O | 2-F | 1 |
| II-31 | —OEt | (R)—Me | CF₃ | 6-fluoropyridin-3-yl | O | 2-F | 1 |
| II-32 | CF₃CH₂SO₂NH— | H | CF₃ | 6-fluoropyridin-3-yl | O | 2-F | 1 |
| II-33 | Me₂NSO₂NH— | H | CF₃ | 6-fluoropyridin-3-yl | O | 2-F | 1 |
| II-34 | MeO(O)CCH₂NH— | H | CF₃ | 6-fluoropyridin-3-yl | O | 2-F | 1 |
| II-35 | —OEt | H | Br | 6-fluoropyridin-3-yl | S | — | 0 |
| II-36 | —OMe | (R)—Me | Br | 6-fluoropyridin-3-yl | O | 2-F | 1 |
| II-37 | —NH—CH₂C(O)OMe | H | Br | 5-fluoropyridin-3-yl | O | 2-Me | 1 |
| II-38 | —OH | H | Br | 5-fluoropyridin-3-yl | O | 2-Me | 1 |
| II-39 | —OCH₂—pyridin-2-yl | H | Br | 5-fluoropyridin-3-yl | O | 2-Me | 1 |
| II-40 | —OCH₂CH₂OMe | H | Br | 5-fluoropyridin-3-yl | O | 2-Me | 1 |
| II-41 | —OCH₂CH₂C(O)OMe | H | Br | 5-fluoropyridin-3-yl | O | 2-Me | 1 |
| II-42 | —OH | H | Cl | 5-fluoropyridin-3-yl | O | 2-Me | 1 |
| II-43 | —OCH₂-tetrahydrofuran- | H | Br | 5-fluoropyridin-3-yl | O | 2-Me | 1 |
| | 2-yl | | | | | | |
| II-44 | —OMe | H | Cl | 5-fluoropyridin-3-yl | O | 2-Me | 1 |
| II-45 | —OEt | H | Br | 5-fluoropyridin-3-yl | O | 2-Me | 1 |
| II-46 | —OEt | H | Br | 6-fluoropyridin-3-yl | O | 2-Me | 1 |

TABLE III
Q = Q3 in formula (I)

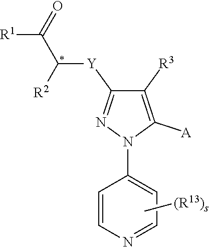

The linkage of R1 is indicated either by “-” or “#”.

| Example | | | | | | | |
|---|---|---|---|---|---|---|---|
| number | R¹ | R² | R³ | R⁴ | Y | R¹² | s |
| III-01 | —OEt | H | Br | 6-fluoropyridin-3-yl | O | 2-F | 1 |
| III-02 | —OH | H | Br | 6-fluoropyridin-3-yl | O | 2-F | 1 |
| III-03 | —OH | (R)—Me | Br | 6-fluoropyridin-3-yl | O | 2-F | 1 |
| III-04 | —OEt | (R)—Me | Br | 6-fluoropyridin-3-yl | C | 2-F | 1 |
| III-05 | CF₃CH₂SO₂NH— | H | Br | 6-fluoropyridin-3-yl | O | 2-F | 1 |
| III-06 | Me₂NSO₂NH— | H | Br | 6-fluoropyridin-3-yl | O | 2-F | 1 |
| III-07 | Me(O)CCH₂NH— | H | Br | 6-fluoropyridin-3-yl | O | 2-F | 1 |
| III-08 | —OEt | H | Br | 6-fluoropyridin-3-yl | O | — | 0 |
| III-09 | —OH | H | Br | 6-fluoropyridin-3-yl | O | — | 0 |
| III-10 | —OH | (R)—Me | Br | 6-fluoropyridin-3-yl | O | — | 0 |
| III-11 | —OEt | (R)—Me | Br | 6-fluoropyridin-3-yl | O | — | 0 |
| III-12 | CF₃CH₂SO₂NH- | H | Br | 6-fluoropyridin-3-yl | O | — | 0 |
| III-13 | Me₂NSO₂NH- | H | Br | 6-fluoropyridin-3-yl | O | — | 0 |
| III-14 | MeO(O)CCH₂NH- | H | Br | 6-fluoropyridin-3-yl | O | 2-H | 0 |
| III-15 | —OEt | H | Br | pyrimidin-5-yl | O | 2-F | 1 |
| III-15 | —OEt | H | Br | pyrimidin-5-yl | S | 2-F | 1 |
| III-16 | —OH | H | Br | pyrimidin-5-yl | S | 2-F | 1 |
| III-17 | 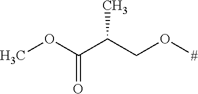 | H | Br | 6-fluoropyridin-3-yl | O | 2-Cl | 1 |
| III-18 | 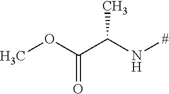 | H | Br | 6-fluoropyridin-3-yl | O | 2-Cl | 1 |
| III-19 | —OCH₂CH₂C(O)OMe | H | Br | 6-fluoropyridin-3-yl | O | 2-Cl | 1 |
| III-20 | CF₃CH₂SO₂NH— | H | Br | 6-fluoropyridin-3-yl | C | 2-Cl | 1 |
| III-21 | 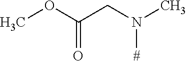 | H | Br | 6-fluoropyridin-3-yl | O | 2-Cl | 1 |
| III-22 | —OMe | (R)—Me | Br | 6-fluoropyridin-3-yl | O | 2-Cl | 1 |
| III-23 | —OH | H | Br | 6-fluoropyridin-3-yl | O | 2-Cl | 1 |
| III-24 | —OEt | H | Br | 6-fluoropyridin-3-yl | O | 2-Cl | 1 |
| III-25 | 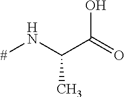 | H | Br | 6-fluoropyridin-3-yl | O | 2-F | 1 |
| III-26 | 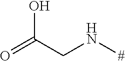 | H | Br | 6-fluoropyridin-3-yl | O | 2-F | 1 |
| III-27 | —OCH₂CH₂C(O)OMe | H | Br | 6-fluoropyridin-3-yl | O | 2-F | 1 |
| III-28 | 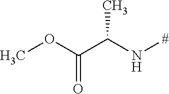 | H | Br | 6-fluoropyridin-3-yl | O | 2-F | 1 |
| III-29 | —OEt | H | Br | 6-fluoropyridin-3-yl | O | 3-F | 1 |
| III-30 | —OH | (R)—Me | Br | 6-fluoropyridin-3-yl | O | 2-Cl | 1 |

A further aspect of the invention relates to the preparation of the inventive compounds of the general formula (I). The compounds of the invention can be prepared in various ways.

The inventive compounds of the general formula (Tb) are synthesized, as shown in Scheme 1, via an amide coupling of an inventive acid of the general formula (Ta) with an amine of the general formula (TI) in the presence of an amide coupling reagent, for example T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in a suitable solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene. For T3P peptide coupling conditions see Organic Process Research & Development 2009, 13, 900-906.

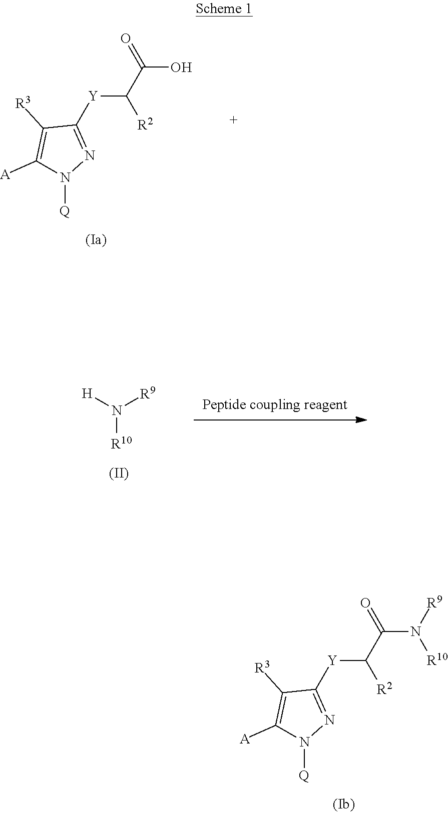

The acids of the general formula (Ia) can be prepared by hydrolysis of the inventive esters of the general formula (Ic) by or analogously to the standard methods that are well known to the person skilled in the art (Scheme 2). The ester hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

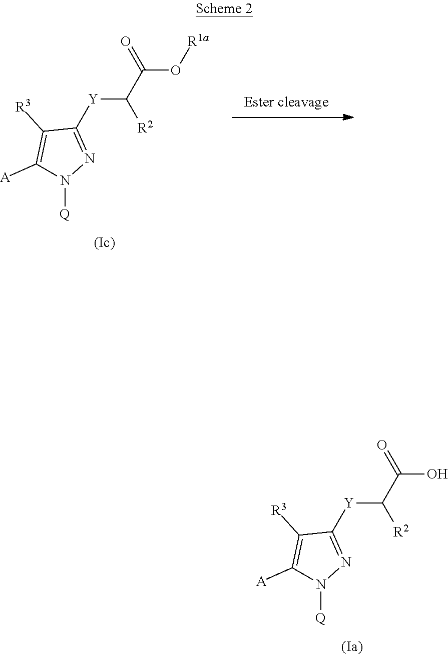

The compound of the general formula (Ic) can be synthesized, for example, by alkylation of the compound of the general formula (III) with a halide of the general formula (IV) in the presence of a base, by or analogously to methods known to the person skilled in the art (see Scheme 3). As base, preference is given to a carbonate salt of an alkali metal selected from the group consisting of lithium, sodium, potassium and caesium. The reaction preferably takes place within the temperature range between room temperature and 150° C. in an appropriate solvent, for example dichloromethane, acetonitrile, N,N-dimethylformamide or diiodomethane. See J. Med. Chem. 2011, 54(16), 5820-5835 and WO2010/010154. The “X” radical in the compound of the general formula (IV) is preferably chlorine, bromine or iodine.

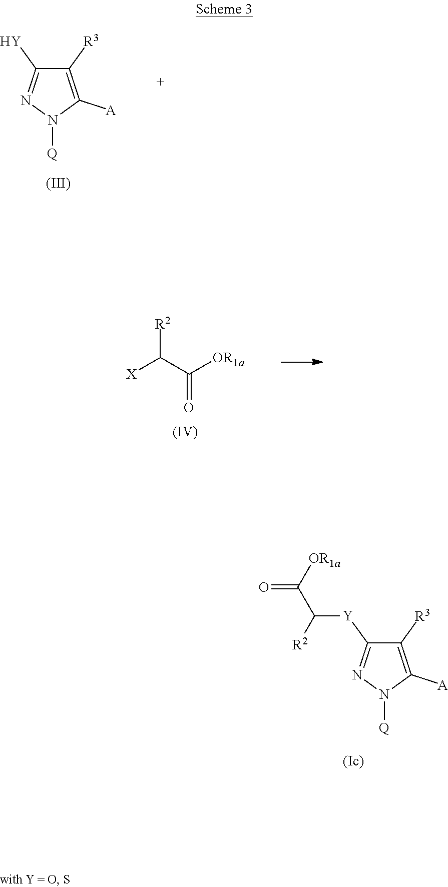

Scheme 4 describes the synthesis of the compound of the general formula (VI) by reaction of a pyrazolone of the general formula (V) in the presence of a sulfurizing reagent, for example phosphorus pentasulfide or Lawesson's reagent in an appropriate solvent, for example toluene.

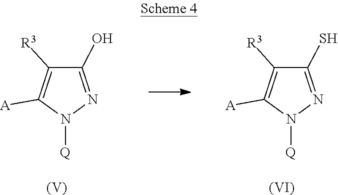

Scheme 5 describes the synthesis of the compound of the general formula (V, with R³═Cl, Br, I) by reaction of a pyrazole of the general formula (VII) with an electrophilic halogenating reagent of the general formula (VIII), for example N-chlorosuccinimide (VIII, X═Cl), N-bromosuccinimide (VIII, X═Br) or N-iodosuccinimide (VIII, X═I). The reaction preferably takes place within the temperature range between 0° C. and 120° C. in an appropriate solvent, for example N,N-dimethylformamide, 1,2-dichloroethane or acetonitrile.

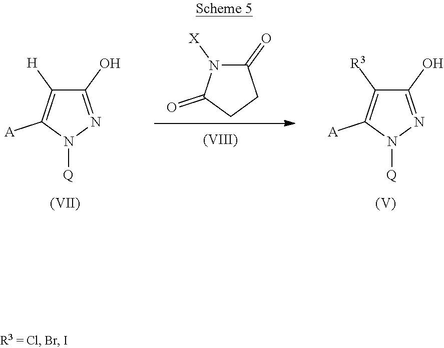

In an analogous manner, it is also possible to use other electrophilic reagents, for example electrophilic nitrating reagents such as nitrating acid, nitronium tetrafluoroborate or ammonium nitrate/trifluoroacetic acid (when R³═NO₂) or electrophilic fluorinating reagents, such as DAST, Selectfluor or N-fluorobenzenesulfonimide (when R³═F). These reactions preferably take place within a temperature range between 0° C. and 120° C. in an appropriate solvent, for example N,N-dimethylformamide, 1,2-dichloroethane or acetonitrile.

The compounds of the general formula (V, with R³═Cl, Br, I, preferably R³═Br, I) that are described in Scheme 5 can be used analogously to methods that are well known to the person skilled in the art, for example Suzuki coupling, together with a reagent of the formula R³—B(OR^{b})(OR^{c}) where the R^{b} and R^{c} radicals are independently, for example, hydrogen, (C₁-C₄)-alkyl, or, when the R^{b} and R^{c} radicals are joined to one another, are collectively ethylene or propylene, to prepare further compounds of the general formula (V) in which R³ is defined, for example, as (C₁-C₆)-alkyl, (C₂-C₆)-alkenyl or (C₃-C₆)-cycloalkyl, especially cyclopropyl.

Scheme 6 describes the synthesis of the compound of the general formula (Id) by reaction of a pyrazole of the general formula (L) with a halosuccinimide of the general formula (VIII) in an appropriate solvent, for example N,N-dimethylformamide.

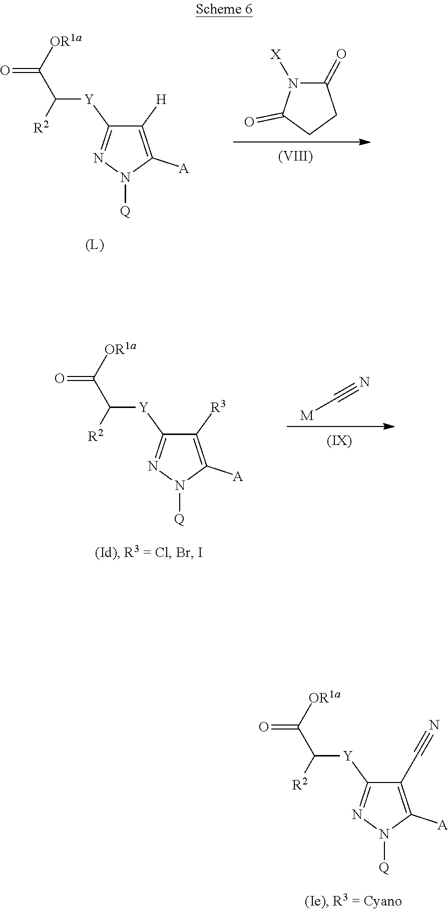

A compound of the general formula (Ie) can be prepared, for example, by reaction of a compound of the formula (Id) in a suitable solvent with a metal cyanide M-CN (IX) with addition of an appropriate amount of a transition metal catalyst, especially palladium catalysts such as palladium(0)tetrakis(triphenylphosphine) or palladium diacetate or bis(triphenylphosphine)palladium(II) dichloride or nickel catalysts such as nickel(II) acetylacetonate or bis(triphenylphosphine)nickel(II) chloride, preferably at elevated temperature in an organic solvent, for example 1,2-dimethoxyethane or N,N-dimethylformamide (Scheme 6). The “M” radical represents, for example, magnesium, zinc, lithium or sodium. Cross-coupling methods that are suitable in general are those described in R. D. Larsen, Organometallics in Process Chemistry 2004 Springer Verlag, in I. Tsuji, Palladium Reagents and Catalysts 2004 Wiley, and in M. Beller, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

The compounds of the general formula (Id, with R³═Cl, Br, I, preferably R³═Br, I) that are described in Scheme 6 can be used analogously to methods that are well known to the person skilled in the art, for example Suzuki coupling, together with a reagent of the formula R³—B(OR^{b})(OR^{c}) where the R^{b} and R^{c} radicals are independently, for example, hydrogen, (C₁-C₄)-alkyl, or, when the R^{b} and R^{c} radicals are joined to one another, are collectively ethylene or propylene, to prepare further inventive compounds in which R³ is defined, for example, as (C₁-C₆)-alkyl, (C₂-C₆)-alkenyl or (C₃-C₆)-cycloalkyl, especially cyclopropyl.

The 3-hydroxypyrazoles (V) can be prepared analogously to methods known from the literature from substituted 3-azinylpropynoic acid derivatives (X) and pyridyl hydrazines (XI) (Scheme 7; e.g.: Adv. Synth. Catal. 2014, 356, 3135-3147) or from substituted azinylacrylic acid derivatives (XIV) and pyridyl hydrazines (Scheme 8; e.g.: J. Heterocyclic Chem., 49, 130 (2012)).

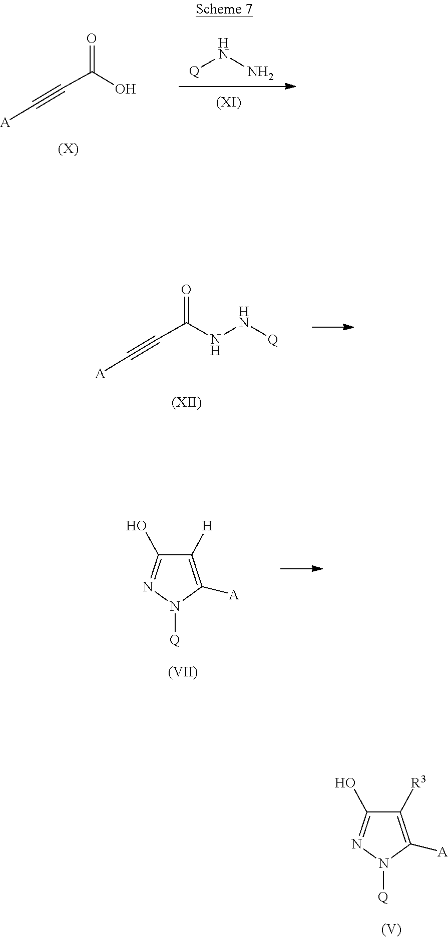

Scheme 7 describes the synthesis of the compound of the general formula (V) from substituted 3-azinylpropynoic acid derivatives (X) and pyridyl hydrazines (XI).

The hydroxypyrazoles (VII) are converted to the hydroxypyrazoles (V) as described in Scheme 5 above. The 3-hydroxypyrazoles of the general formula (VII) are synthesized by reaction of the compounds of the general formula (XII) in the presence of a copper halide, for example copper(I) iodide, copper(I) bromide, or a base such as sodium methoxide or an acid such as methanesulfonic acid to give 3-hydroxypyrazoles of the general formula (VII). The reaction preferably takes place in the temperature range between 0° C. and 120° C., in an appropriate solvent, for example 1,2-dichloroethane, acetonitrile, N,N-dimethylformamide, n-propanol or ethyl acetate. Preferably, the reaction takes place in N,N-dimethylformamide.

The compounds of the general formula (XII) are synthesized via an amide coupling of an acid of the general formula (X) with a pyridyl hydrazine of the general formula (XI) in the presence of an amide coupling reagent, for example T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in an appropriate solvent, for example dichloromethane, tetrahydrofuran, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 7). For T3P peptide coupling conditions see Organic Process Research & Development 2009, 13, 900-906.

Scheme 8 shows the synthesis of 3-hydroxypyrazoles of the general formula (VII) from substituted azinylacrylic acid derivatives of the formula (XIV) and pyridyl hydrazines of the formula (XI).

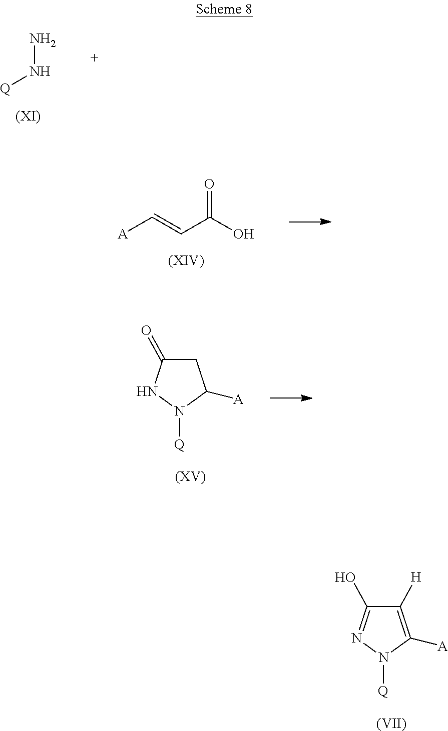

Compounds of the general formula (XV) can be synthesized via an amide coupling of a substituted propenoic acid of the general formula (XIV) with a pyridyl hydrazine of the general formula (XI) in the presence of an amide coupling reagent, for example T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide. The reaction takes place preferably within the temperature range between 0° C. and 80° C., in a suitable solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene. The 3-hydroxypyrazoles of the general formula (VII) are synthesized by reaction of the compounds of the general formula (XV) in the presence of an iron halide such as iron(III) chloride. The reaction preferably takes place in the temperature range between 0° C. and 120° C. in an appropriate solvent such as 1,2-dichloroethane, acetonitrile, N,N-dimethylformamide or ethyl acetate.

Compounds of the general formula (XVI) can be synthesized by N-arylation of a 3-hydroxypyrazole of the general formula (XIII) with a pyridyl halide of the formula (M) in the presence of a copper halide, for example copper(I) iodide. The reaction takes place preferably within the temperature range between 0° C. and 120° C., in an appropriate solvent, for example acetonitrile or N,N-dimethylformamide, and in the presence of a base, for example triethylamine, caesium carbonate (Scheme 9).

The compounds of the general formula (XIII) can be prepared by or analogously to methods known to the person skilled in the art (e.g., Chem. Med. Chem. 2015, 10, 1184-1199). The “X” radical in formula (M) is, for example, chlorine, bromine or iodine, preferably bromine or iodine.

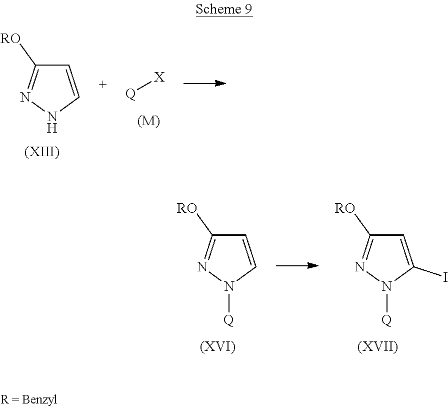

The 5-iodopyrazoles of the general formula (XVII) are synthesized by reaction of the compounds of the general formula (XVI) in the presence of a lithium base, for example lithium diisopropylamide, and iodine. The reaction preferably takes place within the temperature range between −78° C. and −60° C., in an appropriate solvent, for example diethyl ether or tetrahydrofuran.

Compounds of the formula (VII) can also be prepared, for example, by reaction of a compound of the general formula (XVII) in a suitable solvent with a reagent M-A, with addition of an appropriate amount of a transition metal catalyst, especially a palladium catalyst such as palladium diacetate or bis(triphenylphosphine)palladium(II) dichloride, or a nickel catalyst such as nickel(II) acetylacetonate or bis(triphenylphosphine)nickel(II) chloride, preferably at elevated temperature in an organic solvent such as 1,2-dimethoxyethane. The “M” radical represents, for example, Mg-Hal, Zn-Hal, Sn((C₁-C₄)-alkyl)₃, lithium, copper or B(OR^{b})(OR^{c}), where the R^{b} and R^{c} radicals are independently, for example, hydrogen, (C₁-C₄)-alkyl, or, if the R^{b} and R^{c} radicals are bonded to one another, they are collectively ethylene or propylene (Scheme 10).

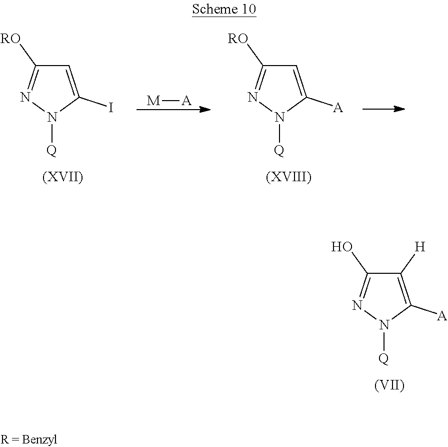

Alternatively, inventive compounds of the formula (Ic) can also be prepared as shown in Scheme 11 proceeding from 5-aminopyrazoles of the general formula (XIX).

A compound of the formula (Ic) can be prepared, for example, by reaction of a compound of the formula (XXI) in a suitable solvent with a reagent M-A, with addition of an appropriate amount of a transition metal catalyst, especially a palladium catalyst such as palladium diacetate or bis(triphenylphosphine)palladium(II) dichloride, or a nickel catalyst such as nickel(II) acetylacetonate or bis(triphenylphosphine)nickel(II) chloride, preferably at elevated temperature in an organic solvent such as 1,2-dimethoxyethane. The “M” radical represents, for example, Mg-Hal, Zn-Hal, Sn((C₁-C₄)-alkyl)₃, lithium, copper or B(OR^{b})(OR^{c}), where the R^{b} and R^{c} radicals are independently, for example, hydrogen, (C₁-C₄)-alkyl, or, if the R^{b} and R^{c} radicals are bonded to one another, they are collectively ethylene or propylene.

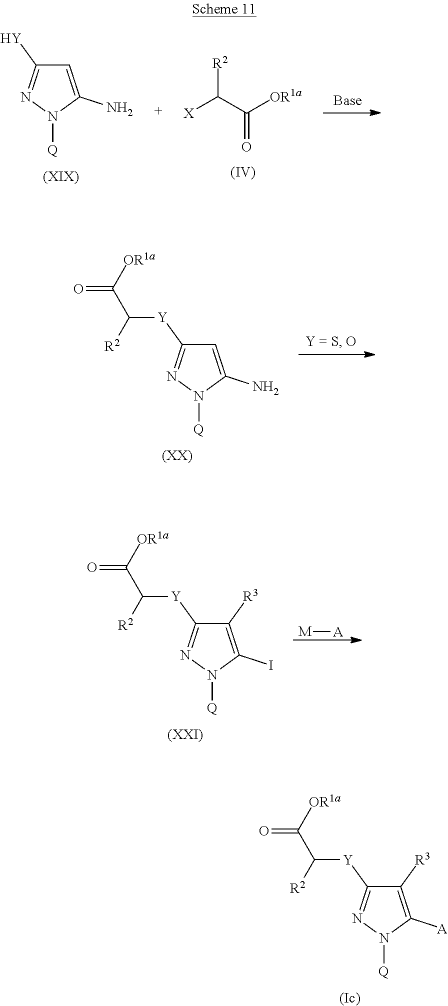

Compounds of the general formula (XXI) can be prepared by a diazotization and subsequent Sandmeyer reaction from 5-aminopyrazoles of the general formula (XX) with the customary organic and inorganic nitrites, for example 1,1-dimethylethyl nitrite, tert-butyl nitrite or isoamyl nitrite, in the presence of usable iodination reagents, for example iodine or diiodomethane (Scheme 11). The reaction preferably takes place within a temperature range between room temperature and 0° C. and 120° C. in an appropriate solvent, for example dichloromethane, acetonitrile, N,N-dimethylformamide. The compound of the general formula (XX) can be prepared, for example, by alkylation of the compound of the general formula (XIX) with a halide of the general formula (IV) in the presence of a base, by or analogously to methods known to the person skilled in the art. The “X” radical in formula (IV) is, for example, chlorine, bromine or iodine. The base may be a carbonate salt of an alkali metal (for example lithium, sodium, potassium or caesium), and the reaction preferably takes place within a temperature range between room temperature and 150° C. in an appropriate solvent, for example dichloromethane, acetonitrile, N,N-dimethylformamide or ethyl acetate.

Scheme 12 describes the synthesis of the compound of the general formula (XXIII) by reaction of a pyrazolone of the general formula (XXII) in the presence of a sulfurizing reagent, for example phosphorus pentasulfide or Lawesson's reagent in an appropriate solvent, for example toluene. The compounds of the general formula (XXII) are commercially available or are preparable analogously to methods known to the person skilled in the art.

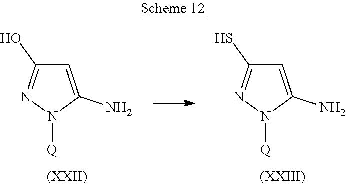

The compound of the general formula (Id) can be prepared by reaction of a 3-aminopyrazole of the general formula (XXIV) with a disulfide of the general formula (XXV) in the presence of an organic nitrite, for example 1,1-dimethylethyl nitrite, tert-butyl nitrite or isoamyl nitrite, in the presence of a metal, for example copper (see Scheme 13). The reaction preferably takes place within the temperature range between room temperature and 120° C. in an appropriate solvent, for example dichloromethane, acetonitrile, N,N-dimethylformamide or 1,2-dichloroethane.

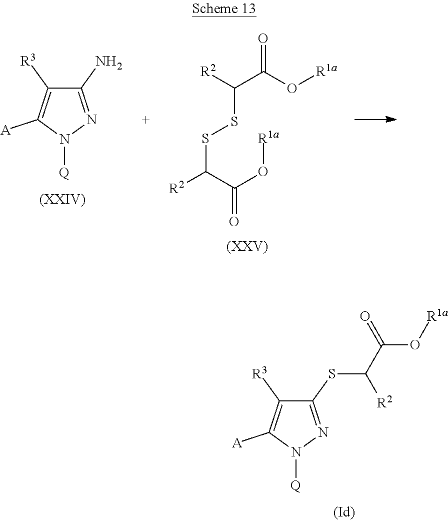

With R^{1a}═(C₁-C₄)-alkyl.

The 3-aminopyrazole of the general formula (XXIV) can be synthesized by reaction of the compound of the general formula (XXVI) in the presence of a Lewis acid, for example trifluoroacetic acid, by or analogously to methods known to the person skilled in the art (Scheme 14). The reaction preferably takes place within the temperature range between room temperature and 140° C.

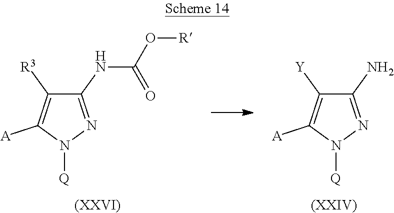

With R′═(C₁-C₄)-alkyl

The compounds of the general formula (XXVI) are synthesized via a Curtius reaction of an acid of the general formula (XXVII) with an azide of the general formula (XXVIII). The reaction takes place preferably within the temperature range between 0° C. and 100° C., in an appropriate solvent, for example tert-butanol, and in the presence of a base, for example triethylamine, N,N-diisopropylethylamine or 1,8-diazabicyclo[5.4.0]undec-7-ene (see Scheme 15).

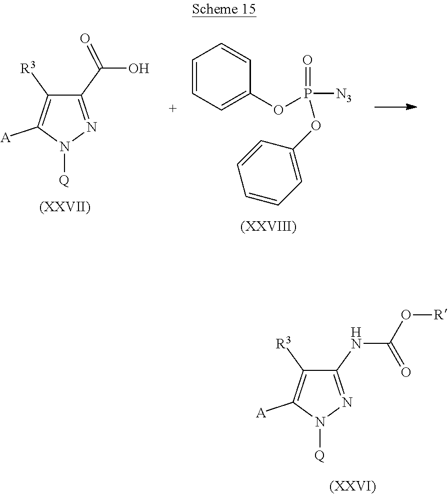

The acid of the general formula (XXVII) can be prepared by hydrolysis of the compound of the general formula (XXIX) by or analogously to methods known to the person skilled in the art (Scheme 16). The hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

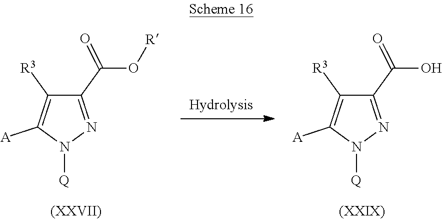

With R′═(C₁-C₄)-alkyl.

Scheme 17 describes the synthesis of the compound of the general formula (XXXI) by reaction of a pyrazole of the general formula (XXX) with a halosuccinimide of the general formula (VIII) in an appropriate solvent, for example DMF.

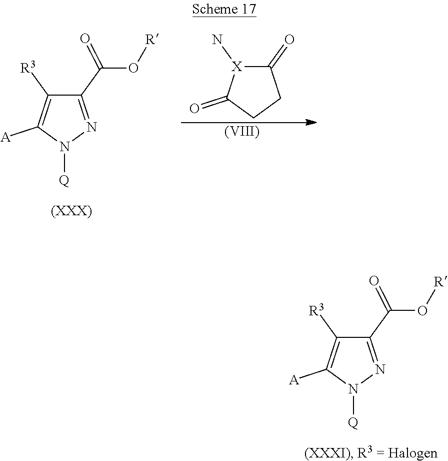

With X=halogen.

The compounds of the general formula (XXXIII) are synthesized via a condensation of a diketo ester of the general formula (XXXII) with a pyridyl hydrazine of the general formula (XI) in the presence of a Brønsted acid, for example acetic acid or hydrogen chloride, in an appropriate solvent, for example methanol, ethanol, isopropanol, n-butanol, tetrahydrofuran, dioxane, toluene or chlorobenzene (Scheme 18). The reaction preferably takes place in the temperature range between 0° C. and 150° C. The compounds of the general formulas (XI) and (XXXI) are commercially available or can be prepared analogously to methods known to the person skilled in the art.

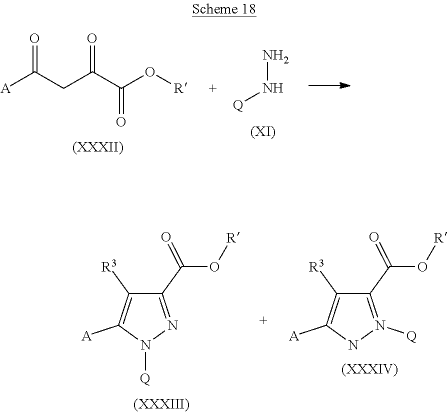

With R′═(C₁-C₄)-alkyl.

Compounds of the general formulae (XXXIV) and (XXXV) can be prepared by reaction of a compound of the formula (XXXIII) in the presence of an oxidizing agent, for example mCPBA (3-chloroperbenzoic acid), in an appropriate solvent, for example dichloromethane or 1,2-dichloroethane (Scheme 19). The reaction preferably takes place in the temperature range between −30° C. and 100° C.

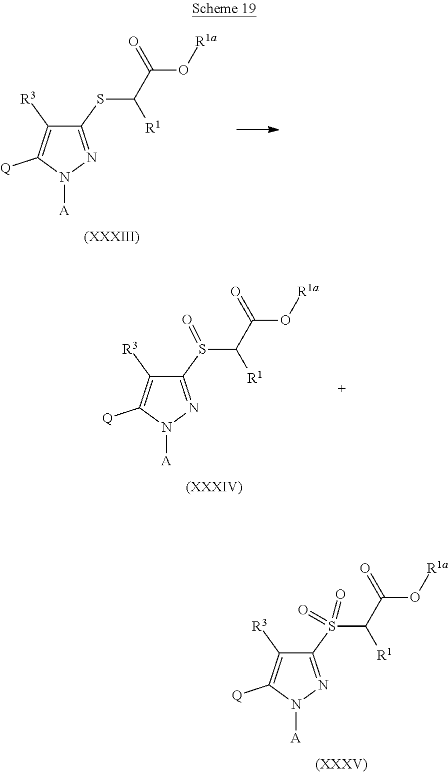

The inventive compounds of the formula (I) (and/or salts thereof), referred to collectively as “compounds of the invention” hereinafter, have excellent herbicidal efficacy against a broad spectrum of economically important monocotyledonous and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377 A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A)
genetically modified plants having reduced photorespiration, which have higher yields and higher stress tolerance (EP 0305398 A)
transgenic crop plants which produce pharmaceutically or diagnostically important proteins (“molecular pharming”)
transgenic crop plants which feature higher yields or better quality
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. For the connection of the DNA fragments to one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, NY; or Winnacker “Gene und Klone” [Genes and Clones], VCH Weinheim, 2nd edition, 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Obtainable in this way are transgenic plants having properties altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients, or to any desired combinations of these active ingredients.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as corn or soya with the trade name or the designation Optimum™ GAT™ (glyphosate ALS tolerant), for example.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Küchler, “Chemische Technologie” [Chemical Technology], Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The necessary formulation auxiliaries such as inert materials, surfactants, solvents and further additives are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesell., Stuttgart 1976, Winnacker-Küchler, “Chemische Technologie”, volume 7, C. Hanser Verlag Munich, 4th ed. 1986.

On the basis of these formulations, it is also possible to produce combinations with other active ingredients, for example insecticides, acaricides, herbicides, fungicides, and also with safeners, fertilizers and/or growth regulators, for example in the form of a finished formulation or as a tank mix.

Combination partners usable for the compounds of the invention in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as known, for example, from Weed Research 26 (1986) 441-445 or “The Pesticide Manual”, 16th edition, The British Crop Protection Council and the Royal Soc. of Chemistry, 2006, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Combination partners usable for the compounds of the general formula (I) in mixed formulations or in a tankmix are, for example, known active ingredients that are based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase or act as plant growth regulators, as known, for example, from Weed Research 26 (1986) 441-445 or “The Pesticide Manual”, 14th edition, The British Crop Protection Council and the Royal Soc. of Chemistry, 2006, and literature cited therein.

Examples of known herbicides or plant growth regulators which can be combined with compounds of the general formula (I) include the active ingredients which follow (the compounds are designated either by the “common name” according to the International Organization for Standardization (ISO) or by the chemical name or by the code number) and always encompass all use forms, such as acids, salts, esters and isomers, such as stereoisomers and optical isomers. These include, by way of example, one use form and in some cases also a plurality of use forms:
acetochlor, acifluorfen, acifluorfen-methyl, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, aminopyralid-dimethylammonium, aminopyralid-tripromine, amitrol, ammonium sulfamat, anilofos, asulam, asulam-potassium, asulam-sodium, atrazin, azafenidin, azimsulfuron, beflubutamid, (S)-(−)-beflubutamid, beflubutamid-M, benazolin, benazolin-ethyl, benazolin-dimethylammonium, benazolin-potassium, benfluralin, benfuresat, bensulfuron, bensulfuron-methyl, bensulid, bentazon, bentazon-sodium, benzobicyclon, benzofenap, bicyclopyron, bifenox, bilanafos, bilanafos-sodium, bipyrazon, bispyribac, bispyribac-sodium, bixlozon, bromacil, bromacil-lithium, bromacil-sodium, bromobutid, bromofenoxim, bromoxynil, bromoxynil-butyrate, bromoxynil-potassium, bromoxynil-heptanoate and bromoxynil-octanoate, busoxinon, butachlor, butafenacil, butamifos, butenachlor, butralin, butroxydim, butylat, cafenstrol, cambendichlor, carbetamid, carfentrazon, carfentrazon-ethyl, chloramben, chloramben-ammonium, chloramben-diolamine, chlroamben-methyl, chloramben-methylammonium, chloramben-sodium, chlorbromuron, chlorfenac, chlorfenac-ammonium, chlorfenac-sodium, chlorfenprop, chlorfenprop-methyl, chlorflurenol, chlorflurenol-methyl, chloridazon, chlorimuron, chlorimuron-ethyl, chlorophthalim, chlorotoluron, chlorsulfuron, chlorthal, chlorthal-dimethyl, chlorthal-monomethyl, cinidon, cinidon-ethyl, cinmethylin, exo-(+)-cinmethylin, i.e. (1R,2S,4S)-4-isopropyl-1-methyl-2-[(2-methylbenzyl)oxy]-7-oxabicyclo[2.2.1]-heptane, exo-(−)-cinmethylin, i.e. (1R,2S,4S)-4-isopropyl-1-methyl-2-[(2-methylbenzyl)oxy]-7-oxabicyclo[2.2.1]heptane, cinosulfuron, clacyfos, clethodim, clodinafop, clodinafop-ethyl, clodinafop-propargyl, clomazone, clomeprop, clopyralid, clopyralid-methyl, clopyralid-olamine, clopyralid-potassium, clopyralid-tripomine, cloransulam, cloransulam-methyl, cumyluron, cyanamid, cyanazin, cycloat, cyclopyranil, cyclopyrimorat, cyclosulfamuron, cycloxydim, cyhalofop, cyhalofop-butyl, cyprazin, 2,4-D (and also the ammonium, butotyl, butyl, choline, diethylammonium, dimethylammonium, diolamine, doboxyl, dodecylammonium, etexyl, ethyl, 2-ethylhexyl, heptylammonium, isobutyl, isooctyl, isopropyl, isopropylammonium, lithium, meptyl, methyl, potassium, tetradecylammonium, triethylammonium, triisopropanolammonium, tripromine and trolamine salts thereof), 2,4-DB, 2,4-DB-butyl, 2,4-DB-dimethylammonium, 2,4-DB-isooctyl, 2,4-DB-potassium and 2,4-DB-sodium, daimuron (dymron), dalapon, dalapon-calcium, dalapon-magnesium, dalapon-sodium, dazomet, dazomet-sodium, n-decanol, 7-deoxy-d-sedoheptulose, desmedipham, detosyl-pyrazolate (dTP), dicamba and salts thereof, e.g. dicamba-biproamine, dicamba-N,N-bis(3-aminopropyl)methylamine, dicamba-butotyl, dicamba-choline, dicamba-diglycolamine, dicamba-dimethylammonium, dicamba-diethanolamineammonium, dicamba-diethylammonium, dicamba-isopropylammonium, dicamba-methyl, dicamba-monoethanolamine, dicamba-olamine, dicamba-potassium, dicamba-sodium, dicamba-triethanolamine, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-butotyl, dichlorprop-dimethylammonium, dichlorprop-etexyl, dichlorprop-ethylammonium, dichlorprop-isoctyl, dichlorprop-methyl, dichlorprop-potassium, dichlorprop-sodium, dichlorprop-P, dichlorprop-P-dimethylammonium, dichlorprop-P-etexyl, dichlorprop-P-potassium, dichlorprop-sodium, diclofop, diclofop-methyl, diclofop-P, diclofop-P-methyl, diclosulam, difenzoquat, difenzoquat-metilsulfate, diflufenican, diflufenzopyr, diflufenzopyr-sodium, dimefuron, dimepiperate, dimesulfazet, dimethachlor, dimethametryn, dimethenamid, dimethenamid-P, dimetrasulfuron, dinitramine, dinoterb, dinoterb-acetate, diphenamid, diquat, diquat-dibromid, diquat-dichloride, dithiopyr, diuron, DNOC, DNOC-ammonium, DNOC-potassium, DNOC-sodium, endothal, endothal-diammonium, endothal-dipotassium, endothal-disodium, epyrifenacil (S-3100), ePTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-5231, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenpyrazone, fenquinotrione, fentrazamid, flamprop, flamprop-isoproyl, flamprop-methyl, flamprop-M-isopropyl, flamprop-M-methyl, flazasulfuron, florasulam, florpyrauxifen, florpyrauxifen-benzyl, fluazifop, fluazifop-butyl, fluazifop-methyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanat, flupropanat-sodium, flupyrsulfuron, flupyrsulfuron-methyl, flupyrsulfuron-methyl-sodium, fluridon, flurochloridon, fluroxypyr, fluroxypyr-butometyl, fluroxypyr-meptyl, flurtamon, fluthiacet, fluthiacet-methyl, fomesafen, fomesafen-sodium, foramsulfuron, foramsulfuron-sodium, fosamine, fosamine-ammonium, glufosinate, glufosinate-ammonium, glufosinate-sodium, L-glufosinate-ammonium, L-glufosinate-sodium, glufosinate-P-sodium, glufosinate-P-ammonium, glyphosate, glyphosate-ammonium, glyphosate-isopropylammonium, glyphosate-diammonium, glyphosate-dimethylammonium, glyphosate-potassium, glyphosate-sodium, glyphosate-sesquisodium and glyphosate-trimesium, H-9201, i.e. O-(2,4-dimethyl-6-nitrophenyl)-O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, haloxifop-sodium, hexazinon, HNPC-A8169, i.e. prop-2-yn-1-yl (2S)-2-{3-[(5-tert-butylpyridin-2-yl)oxy]phenoxy}propanoate, HW-02, i.e. 1-(dimethoxyphosphoryl)ethyl (2,4-dichlorophenoxy)acetate, hydantocidin, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazaquin-methyl, imazethapyr, imazethapyr-ammonium, imazosulfuron, indanofan, indaziflam, iodosulfuron, iodosulfuron-methyl, iodosulfuron-methyl-sodium, ioxynil, ioxynil-lithium, -octanoate, -potassium and sodium, ipfencarbazon, isoproturon, isouron, isoxaben, isoxaflutole, karbutilat, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, ketospiradox-potassium, lactofen, lenacil, linuron, MCPA, MCPA-butotyl, -butyl, -dimethylammonium, -diolamine, -2-ethylhexyl, -ethyl, -isobutyl, isoctyl, -isopropyl, -isopropylammonium, -methyl, -olamine, -potassium, -sodium and -trolamine, MCPB, MCPB-methyl, -ethyl and -sodium, mecoprop, mecoprop-butotyl, mecoprop-dimethylammonium, mecoprop-diolamine, mecoprop-etexyl, mecoprop-ethadyl, mecoprop-isoctyl, mecoprop-methyl, mecoprop-potassium, mecoprop-sodium, and mecoprop-trolamine, mecoprop-P, mecoprop-P-butotyl, -dimethylammonium, -2-ethylhexyl and -potassium, mefenacet, mefluidid, mefluidid-diolamine, mefluidid-potassium, mesosulfuron, mesosulfuron-methyl, mesosulfuron-sodium, mesotrion, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-methyl, MT-5950, i.e. N-[3-chloro-4-(1-methylethyl)-phenyl]-2-methylpentanamide, NGGC-011, napropamid, NC-310, i.e. 4-(2,4-dichlorobenzoyl)-1-methyl-5-benzyloxypyrazole, NC-656, i.e. 3-[(isopropylsulfonyl)methyl]-N-(5-methyl-1,3,4-oxadiazol-2-yl)-5-(trifluoromethyl)[1,2,4]triazolo-[4,3-a]pyridine-8-carboxamide, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefone, oxyfluorfen, paraquat, paraquat-dichloride, paraquat-dimethylsulfate, pebulate, pendimethalin, penoxsulam, pentachlorophenol, pentoxazone, pethoxamid, petroleum oil, phenmedipham, phenmedipham-ethyl, picloram, picloram-dimethylammonium, picloram-etexyl, picloram-isoctyl, picloram-methyl, picloram-olamine, picloram-potassium, picloram-triethylammonium, picloram-tripromine, picloram-trolamine, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamid, prosulfocarb, prosulfuron, pyraclonil, pyraflufen, pyraflufen-ethyl, pyrasulfotol, pyrazolynat (pyrazolat), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridat, pyriftalid, pyriminobac, pyriminobac-methyl, pyrimisulfan, pyrithiobac, pyrithiobac-sodium, pyroxasulfon, pyroxsulam, quinclorac, quinclorac-dimethylammonium, quinclorac-methyl, quinmerac, quinoclamin, quizalofop, quizalofop-ethyl, quizalofop-P, quizalofop-P-ethyl, quizalofop-P-tefuryl, QYM201, i.e. 1-{2-chloro-3-[(3-cyclopropyl-5-hydroxy-1-methyl-1H-pyrazol-4-yl)carbonyl]-6-(trifluoromethyl)phenyl}piperidin-2-one, rimsulfuron, saflufenacil, sethoxydim, siduron, simazine, simetryn, SL-261, sulcotrione, sulfentrazone, sulfometuron, sulfometuron-methyl, sulfosulfuron, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid) and salts thereof, e.g. TCA-ammonium, TCA-calcium, TCA-ethyl, TCA-magnesium, TCA-sodrium, tebuthiuron, tefuryltrione, tembotrione, tepraloxydim, terbacil, terbucarb, terbumeton, terbuthylazine, terbutryn, tetflupyrolimet, thaxtomin, thenylchlor, thiazopyr, thiencarbazone, thiencarbazone-methyl, thifensulfuron, thifensulfuron-methyl, thiobencarb, tiafenacil, tolpyralat, topramezon, tralkoxydim, triafamon, tri-allate, triasulfuron, triaziflam, tribenuron, tribenuron-methyl, triclopyr, triclopyr-butotyl, triclopyr-choline, triclopyr-ethyl, triclopyr-triethylammonium, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, ethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 3-chloro-2-[3-(difluoromethyl)isoxazolyl-5-yl]phenyl 5-chloropyrimidin-2-yl ether, 2-(3,4-dimethoxyphenyl)-4-[(2-hydroxy-6-oxocyclohex-1-en-1-yl)carbonyl]-6-methylpyridazin-3(2H)-one, 2-({2-[(2-methoxyethoxy)methyl]-6-methylpyridin-3-yl}carbonyl)cyclohexane-1,3-dione, (5-hydroxy-1-methyl-1H-pyrazol-4-yl)(3,3,4-trimethyl-1,1-dioxido-2,3-dihydro-1-benzothiophen-5-yl)methanone, 1-methyl-4-[(3,3,4-trimethyl-1,1-dioxido-2,3-dihydro-1-benzothiophen-5-yl)carbonyl]-1H-pyrazol-5-yl propane-1-sulfonate, 4-{2-chloro-3-[(3,5-dimethyl-1H-pyrazol-1-yl)methyl]-4-(methylsulfonyl)benzoyl}-1-methyl-1H-pyrazol-5-yl 1,3-dimethyl-1H-pyrazole-4-carboxylate; cyanomethyl 4-amino-3-chloro-5-fluoro-6-(7-fluoro-1H-indol-6-yl)pyridine-2-carboxylate, prop-2-yn-1-yl 4-amino-3-chloro-5-fluoro-6-(7-fluoro-1H-indol-6-yl)pyridine-2-carboxylate, methyl 4-amino-3-chloro-5-fluoro-6-(7-fluoro-1H-indol-6-yl)pyridine-2-carboxylate, 4-amino-3-chloro-5-fluoro-6-(7-fluoro-1H-indol-6-yl)pyridine-2-carboxylic acid, benzyl 4-amino-3-chloro-5-fluoro-6-(7-fluoro-1H-indol-6-yl)pyridine-2-carboxylate, ethyl 4-amino-3-chloro-5-fluoro-6-(7-fluoro-1H-indol-6-yl)pyridine-2-carboxylate, methyl 4-amino-3-chloro-5-fluoro-6-(7-fluoro-1-isobutyryl-1H-indol-6-yl)pyridine-2-carboxylate, methyl 6-(1-acetyl-7-fluoro-1H-indol-6-yl)-4-amino-3-chloro-5-fluoropyridine-2-carboxylate, methyl 4-amino-3-chloro-6-[1-(2,2-dimethylpropanoyl)-7-fluoro-1H-indol-6-yl]-5-fluoropyridine-2-carboxylate, methyl 4-amino-3-chloro-5-fluoro-6-[7-fluoro-1-(methoxyacetyl)-1H-indol-6-yl]pyridine-2-carboxylate, potassium 4-amino-3-chloro-5-fluoro-6-(7-fluoro-1H-indol-6-yl)pyridine-2-carboxylate, sodium 4-amino-3-chloro-5-fluoro-6-(7-fluoro-1H-indol-6-yl)pyridine-2-carboxylate, butyl 4-amino-3-chloro-5-fluoro-6-(7-fluoro-1H-indol-6-yl)pyridine-2-carboxylate, 4-hydroxy-1-methyl-3-[4-(trifluoromethyl)pyridin-2-yl]imidazolidin-2-one, 3-(5-tert-butyl-1,2-oxazol-3-yl)-4-hydroxy-1-methylimidazolidin-2-one, 3-[5-chloro-4-(trifluoromethyl)pyridin-2-yl]-4-hydroxy-1-methylimidazolidin-2-one, 4-hydroxy-1-methoxy-5-methyl-3-[4-(trifluoromethyl)pyridin-2-yl]imidazolidin-2-one, 6-[(2-hydroxy-6-oxocyclohex-1-en-1-yl)carbonyl]-1,5-dimethyl-3-(2-methylphenyl)quinazoline-2,4(1H,3H)-dione, 3-(2,6-dimethylphenyl)-6-[(2-hydroxy-6-oxocyclohex-1-en-1-yl)carbonyl]-1-methylquinazoline-2,4(1H,3H)-dione, 2-[2-chloro-4-(methylsulfonyl)-3-(morpholin-4-ylmethyl)benzoyl]-3-hydroxycyclohex-2-en-1-one, 1-(2-carboxyethyl)-4-(pyrimidin-2-yl)pyridazin-1-ium salt (with appropriate anions, for example chloride, acetate or trifluoroacetate), 1-(2-carboxyethyl)-4-(pyridazin-3-yl)pyridazin-1-ium salt (with appropriate anions, for example chloride, acetate or trifluoroacetate), 4-(pyrimidin-2-yl)-1-(2-sulfoethyl)pyridazin-1-ium salt (with appropriate anions, for example chloride, acetate or trifluoroacetate), 4-(pyridazin-3-yl)-1-(2-sulfoethyl)pyridazin-1-ium salt (with appropriate anions, for example chloride, acetate or trifluoroacetate), 1-(2-carboxyethyl)-4-(1,3-thiazol-2-yl)pyridazin-1-ium salt (with appropriate anions, for example chloride, acetate or trifluoroacetate), 1-(2-carboxyethyl)-4-(1,3-thiazol-2-yl)pyridazin-1-ium salt (with appropriate anions, for example chloride, acetate or trifluoroacetate).

Growth regulators and plant stimulators as mixing partners:
abscisic acid and related analogues [e.g. (2Z,4E)-5-[6-ethynyl-1-hydroxy-2,6-dimethyl-4-oxocyclohex-2-en-1-yl]-3-methylpenta-2,4-dienoic acid, methyl (2Z,4E)-5-[6-ethynyl-1-hydroxy-2,6-dimethyl-4-oxocyclohex-2-en-1-yl]-3-methylpenta-2,4-dienoate, (2Z,4E)-3-ethyl-5-(1-hydroxy-2,6,6-trimethyl-4-oxocyclohex-2-en-1-yl)penta-2,4-dienoic acid, (2E,4E)-5-(1-hydroxy-2,6,6-trimethyl-4-oxocyclohex-2-en-1-yl)-3-(trifluoromethyl)penta-2,4-dienoic acid, methyl (2E,4E)-5-(1-hydroxy-2,6,6-trimethyl-4-oxocyclohex-2-en-1-yl)-3-(trifluoromethyl)penta-2,4-dienoate, (2Z,4E)-5-(2-hydroxy-1,3-dimethyl-5-oxobicyclo[4.1.0]hept-3-en-2-yl)-3-methylpenta-2,4-dienoic acid], acibenzolar, acibenzolar-S-methyl, S-adenosylhomocysteine, allantoin, 2-aminoethoxyvinylglycine (AVG), aminooxyacetic acid and related esters [e.g. (isopropylidene)aminooxyacetic acid 2-(methoxy)-2-oxoethyl ester, (isopropylidene)aminooxyacetic acid 2-(hexyloxy)-2-oxoethyl ester, (cyclohexylidene)aminooxyacetic acid-2-(isopropyloxy)-2-oxoethyl ester], 1-aminocycloprop-1-ylcarboxylic acid, N-methyl-1-aminocyclopropyl-1-carboxylic acid, 1-aminocyclopropyl-1-carboxamide, substituted 1-aminocyclopropyl-1-carboxylic acid derivatives as described in DE3335514, EP30287, DE2906507 or U.S. Pat. No. 5,123,951, 1-aminocyclopropyl-1-hydroxamic acid, 5-aminolevulinic acid, ancymidol, 6-benzylaminopurine, bikinin, brassinolide, brassinolide-ethyl, L-canaline, catechol and catechols (e.g. (2S,3R)-2-(3,4-dihydroxyphenyl)-3,4-dihydro-2H-chromene-3,5,7-triol), chitooligosaccharides (CO; COs differ from LCOs in that they lack the fatty acid side chain characteristic of LCOs. COs, in some cases referred to as N-acetylchitooligosaccharides, are also constructed from GlcNAc units but have side chains that distinguish them from chitin molecules [(C₈H₁₃NO₅)ₙ, CAS No. 1398-61-4] and chitosan molecules [(C₅H₁₁NO₄)ₙ, CAS No. 9012-76-4]), chitin-like compounds, chlormequat chloride, cloprop, cyclanilide, 3-(cycloprop-1-enyl)propionic acid, 1-[2-(4-cyano-3,5-dicyclopropylphenyl)acetamido]cyclohexanecarboxylic acid, 1-[2-(4-cyano-3-cyclopropylphenyl)acetamido]cyclohexanecarboxylic acid, 1-cyclopropenylmethanol, daminozid, dazomet, dazomet-sodium, n-decanol, dikegulac, dikegulac-sodium, endothal, endothal-dipotassum, -disodium, and mono(N,N-dimethylalkylammonium), ethephon, 1-ethylcyclopropene, flumetralin, flurenol, flurenol-butyl, flurenol-methyl, flurprimidol, forchlorfenuron, gibberellic acid, inabenfid, indole-3-acetic acid (IAA), 4-indol-3-ylbutyric acid, isoprothiolan, probenazole, jasmonic acid, jasmonic esters or other derivatives (e.g. jasmonic acid methyl ester, jasmonic acid ethyl ester), lipochitooligosaccharides (LCOs, in some cases also referred to as symbiotic nodulation signals (Nod or Nod factors) or as Myc factors, consist of an oligosaccharide backbone composed of β-1,4-bonded N-acetyl-D-glucosamine residues (“GlcNAc”) with an N-bonded fatty acid side chain fused onto the non-reducing end. As can be inferred from the literature, LCOs differ in the number of GlcNAc units in the backbone structure, in the length and in the degree of saturation of the fatty acid chain, and in the substitution of the reducing and non-reducing sugar units), linoleic acid or derivatives thereof, linolenic acid or derivatives thereof, maleic hydrazide, mepiquat chloride, mepiquat pentaborate, 1-methylcyclopropene, 3-methylcyclopropene, methoxyvinylglycine (MVG), 3′-methylabscisic acid, 1-(4-methylphenyl)-N-(2-oxo-1-propyl-1,2,3,4-tetrahydroquinolin-6-yl)methanesulfonamide and related substituted (tetrahydroquinolin-6-yl)methanesulfonamides, (3E,3aR,8bS)-3-({[(2R)-4-methyl-5-oxo-2,5-dihydrofuran-2-yl]oxy}methylene)-3,3a,4,8b-tetrahydro-2H-indeno[1,2-b]furan-2-one and related lactones as described in EP2248421, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, 4-phenylbutyric acid and salts thereof (e.g. sodium 4-phenylbutanoate, potassium 4-phenylbutanoate), phenylalanine, N-phenylphthalamic acid, prohexadione, prohexadione-calcium, 1-n-propylcyclopropene, putrescine, prohydrojasmone, rhizobitoxin, salicylic acid and methyl salicylate, sarcosine, sodium cycloprop-1-en-1-ylacetate, sodium cycloprop-2-en-1-ylacetate, sodium 3-(cycloprop-2-en-1-yl)propanoate, sodium 3-(cycloprop-1-en-1-yl)propanoate, sidefungin, spermidine, spermine, strigolactone, tecnazene, thidiazuron, triacontanol, trinexapac, trinexapac-ethyl, tryptophan, tsitodef, uniconazole, uniconazole-P, 2-fluoro-N-(3-methoxyphenyl)-9H-purin-6-amine.

Safeners which can be used in combination with the inventive compounds of the formula (I) and optionally in combinations with further active ingredients such as insecticides, acaricides, herbicides, fungicides as listed above are preferably selected from the group consisting of:
S1) Compounds of the formula (S1)

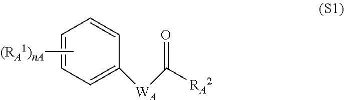

where the symbols and indices are defined as follows:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),

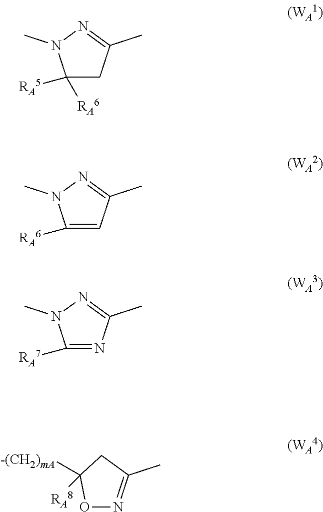

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}S is H, (C₁-C₈)-alkyl, (C₁-C₈)-haloalkyl, (C₁-C₄)-alkoxy-(C₁-C₈)-alkyl, cyano or COOR_{A}⁹, where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
preferably:
a) compounds of the dichlorophenylpyrazoline-3-carboxylic acid type (Sla), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (51-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (51-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (51-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (51-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

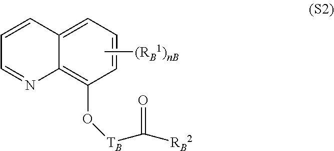

where the symbols and indices have the meanings below:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbon radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2^{a}), preferably
1-methylhexyl (5-chloro-8-quinolinoxy)acetate (“cloquintocet-mexyl”) (S2-1),
(1,3-dimethylbut-1-yl) (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
   b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
   S3) Compounds of the formula (S3)

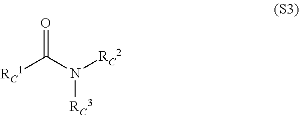

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
preferably:
   active ingredients of the dichloroacetamide type, which are frequently used as pre-emergence safeners (soil-acting safeners), for example
“dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (S3-6),
“AD-67” or “MON 4660” (3-dichloroacetyl-1-oxa-3-azaspiro[4.5]decane) from Nitrokemia or Monsanto (S3-7),
“TI-35” (1-dichloroacetylazepane) from TRI-Chemical RT (S3-8),
“diclonon” (dicyclonon) or “BAS145138” or “LAB145138” (S3-9)
((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10); and
the (R) isomer thereof (S3-11).
   S4) N-acylsulfonamides of the formula (S4) and salts thereof,

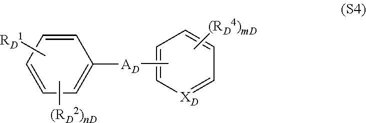

in which the symbols and indices are defined as follows:
A_{D} is SO₂—NR_{D}³—CO or CO—NR_{D}³—SO₂
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven latter radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxyl, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
among these, preference is given to compounds of the N-acylsulfonamide type, for example of the formula (S4^{a}) below, which are known, for example, from WO-A-97/45016

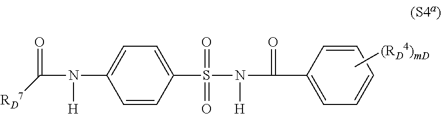

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃;
m_{D} is 1 or 2;
v_{D} is 0, 1, 2 or 3;
and also acylsulfamoylbenzamides, for example of the formula (S4^{b}) below, which are known, for example, from WO-A-99/16744,

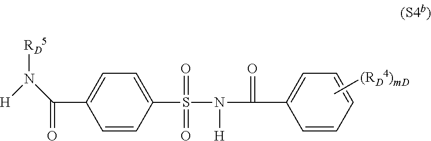

e.g. those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
and also
compounds of the N-acylsulfamoylphenylurea type of the formula (S4^{c}), which are known, for example, from EP-A-365484,

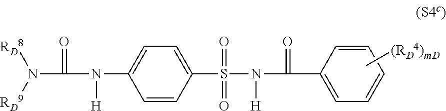

in which
R_{D}⁸ and R_{D}⁹ are independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃,
m_{D} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
   and also
   N-phenylsulfonylterephthalamides of the formula (S4^{d}), which are known, for example, from CN 101838227,

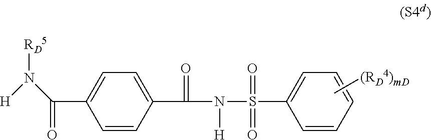

e.g. those in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active ingredients from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

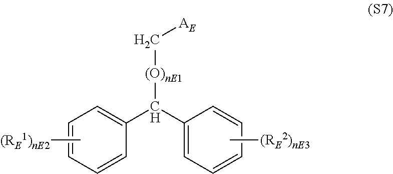

in which the symbols and indices are defined as follows:
R_{E}¹, R_{E}² are independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴
R_{E}³, R_{E}⁴ are independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 Or 1
n_{E}², n_{E}³ are independently 0, 1 or 2,
preferably:
diphenylmethoxyacetic acid,
ethyl diphenylmethoxyacetate,
methyl diphenylmethoxyacetate (CAS reg. no. 41858-19-9) (S7-1).
S8) Compounds of the formula (S8), as described in WO-A-98/27049,

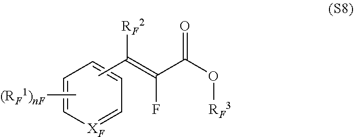

in which
X_{F} is CH or N,
n_{F} in the case that X_{F}═N is an integer from 0 to 4 and
   in the case that X_{F}═CH is an integer from 0 to 5,
R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof, preferably compounds in which
X_{F} is CH,
n_{F} is an integer from 0 to 2,
R^{F} is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy, or salts thereof.
S9) Active ingredients from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S0^{b})
   as described in WO-A-2007/023719 and WO-A-2007/023764

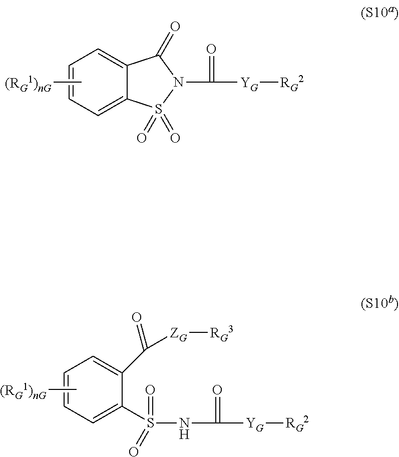

in which
R_{G}¹ is halogen, (C₁-C₄)-alkyl, methoxy, nitro, cyano, CF₃, OCF₃,
Y_{G}, Z_{G} independently of one another represent O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active ingredients of the oxyimino compounds type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
“cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
“naphthalic anhydride” (1,8-naphthalenedicarboxylic anhydride) (S13-1), which is known as a seed-dressing safener for maize against thiocarbamate herbicide damage,
“fenclorim” (4,6-dichloro-2-phenylpyrimidine) (S13-2), which is known as a safener for pretilachlor in sown rice,
“flurazole” (benzyl 2-chloro-4-trifluoromethyl-1,3-thiazole-5-carboxylate) (S13-3), which is known as a seed-dressing safener for millet/sorghum against alachlor and metolachlor damage,
“CL 304415” (CAS Reg. No. 31541-57-8) (4-carboxy-3,4-dihydro-2H-1-benzopyran-4-acetic acid) (S13-4) from American Cyanamid, which is known as a safener for corn against damage by imidazolinones,
“MG 191” (CAS Reg. No. 96420-72-3) (2-dichloromethyl-2-methyl-1,3-dioxolane) (S13-5) from Nitrokemia, which is known as a safener for maize,
“MG 838” (CAS Reg. No. 133993-74-5) (2-propenyl 1-oxa-4-azaspiro[4.5]decane-4-carbodithioate) (S13-6) from Nitrokemia,
“disulfoton” (0,0-diethyl S-2-ethylthioethyl phosphorodithioate) (S13-7),
“dietholate” (0,0-diethyl O-phenyl phosphorothioate) (S13-8),
“mephenate” (4-chlorophenyl methylcarbamate) (S13-9).
S14) Active ingredients which, in addition to herbicidal action against harmful plants, also have safener action on crop plants such as rice, for example
“dimepiperate” or “MY 93” (S-1-methyl 1-phenylethylpiperidine-1-carbothioate), which is known as a safener for rice against damage by the herbicide molinate,
“daimuron” or “SK 23” (1-(1-methyl-1-phenylethyl)-3-p-tolylurea), which is known as a safener for rice against damage by the herbicide imazosulfuron,
“cumyluron”=“JC 940” (3-(2-chlorophenylmethyl)-1-(1-methyl-1-phenylethyl)urea, see JP-A-60087254), which is known as safener for rice against damage by some herbicides,
“methoxyphenone” or “NK 049” (3,3′-dimethyl-4-methoxybenzophenone), which is known as a safener for rice against damage by some herbicides,
“CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

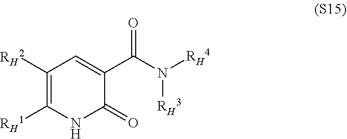

as described in WO-A-2008/131861 and WO-A-2008/131860 in which
R_{H}¹ is a (C₁-C₆)-haloalkyl radical and
R_{H}² is hydrogen or halogen and
R_{H}³, R_{H}⁴ are independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
where each of the 4 latter radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichloro-ethyl).

Particularly preferred safeners are mefenpyr-diethyl, cyprosulfamide, isoxadifen-ethyl, cloquintocet-mexyl, dichlormid and metcamifen.

Wettable powders are preparations uniformly dispersible in water which, in addition to the active ingredient and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1% to 99% by weight, especially 0.1% to 95% by weight, of compounds of the invention. In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partially on whether the active ingredient is in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix. For application, the formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The required application rate of the compounds of the formula (I) and their salts varies according to the external conditions such as, inter alia, temperature, humidity and the type of herbicide used. It can vary within wide limits, for example between 0.001 and 10.0 kg/ha or more of active substance, but it is preferably between 0.005 and 5 kg/ha, more preferably in the range of from 0.01 to 1.5 kg/ha, more preferably in the range of from 0.05 to 1 kg/ha. This applies both to pre-emergence and to post-emergence application.

A carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture. Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, corn cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins, and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions of the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active ingredients can be combined with any solid or liquid additive commonly used for formulation purposes. In general, the compositions and formulations of the invention contain between 0.05% and 99% by weight, 0.01% and 98% by weight, preferably between 0.1% and 95% by weight, more preferably between 0.5% and 90% active ingredient, most preferably between 10 and 70 percent by weight. The active ingredients or compositions of the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, sprayable powders, soluble powders, dusts and granules, water-soluble granules or tablets, water-soluble powders for the treatment of seed, wettable powders, natural products and synthetic substances impregnated with active ingredient, and also microencapsulations in polymeric substances and in coating materials for seed, and also ULV cold-fogging and warm-fogging formulations.

The formulations mentioned can be produced in a manner known per se, for example by mixing the active ingredients with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions of the invention include not only formulations which are already ready for use and can be deployed with a suitable apparatus onto the plant or the seed, but also commercial concentrates which have to be diluted with water prior to use.

The active ingredients of the invention may be present as such or in their (commercial standard) formulations, or else in the use forms prepared from these formulations as a mixture with other (known) active ingredients, such as insecticides, attractants, sterilants, bactericides, acaricides, nematicides, fungicides, growth regulators, herbicides, fertilizers, safeners or semiochemicals.

The inventive treatment of the plants and plant parts with the active ingredients or compositions is effected directly or by action on their surroundings, habitat or storage space by the customary treatment methods, for example by dipping, spraying, atomizing, irrigating, evaporating, dusting, fogging, broadcasting, foaming, painting, spreading-on, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

As also described below, the treatment of transgenic seed with the active ingredients or compositions of the invention is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition of the invention and/or further additives applied to the seed is chosen such that the germination of the seed is not impaired and the plant which arises therefrom is not damaged. This has to be ensured particularly in the case of active ingredients which can exhibit phytotoxic effects at certain application rates.

The compositions of the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417 A, 4,245,432 A, 4,808,430, 5,876,739, US 2003/0176428 A1, WO 2002/080675 A1, WO 2002/028186 A2.

The active ingredients of the invention can be converted to the customary seed-dressing formulations, such as solutions, emulsions, suspensions, powders, foams, slurries or other coating compositions for seed, and also ULV formulations.

These formulations are produced in a known manner, by mixing the active ingredients with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active ingredients. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active ingredients. Preference can be given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active ingredients. Silicone antifoams and magnesium stearate can be used with preference.

Preservatives which may be present in the seed-dressing formulations usable in accordance with the invention are all substances usable for such purposes in agrochemical compositions. Examples include dichlorophen and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active ingredients of the invention, given good plant compatibility, favorable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

Plants which can be treated in accordance with the invention include the following main crop plants: corn, soybean, cotton, Brassica oil seeds such as Brassica napus (e.g. Canola), Brassica rapa, B. juncea (e.g. (field) mustard) and Brassica carinata, rice, wheat, sugar beet, sugar cane, oats, rye, barley, millet and sorghum, triticale, flax, grapes and various fruit and vegetables from various botanic taxa, for example Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes, potatoes, peppers, aubergines), Liliaceae sp., Compositae sp. (for example lettuce, artichokes and chicory—including root chicory, endive or common chicory), Umbelliferae sp. (for example carrots, parsley, celery and celeriac), Cucurbitaceae sp. (for example cucumbers—including gherkins, pumpkins, watermelons, calabashes and melons), Alliaceae sp. (for example leeks and onions), Cruciferae sp. (for example white cabbage, red cabbage, broccoli, cauliflower, Brussels sprouts, pak choi, kohlrabi, radishes, horseradish, cress and chinese cabbage), Leguminosae sp. (for example peanuts, peas, and beans—for example common beans and broad beans), Chenopodiaceae sp. (for example Swiss chard, fodder beet, spinach, beetroot), Malvaceae (for example okra), Asparagaceae (for example asparagus); useful plants and ornamental plants in the garden and woods; and in each case genetically modified types of these plants.

As mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) which have been grown by conventional breeding, by mutagenesis or by recombinant DNA techniques. They may be cultivars, varieties, biotypes and genotypes.

The treatment method of the invention can be used for the treatment of genetically modified organisms (GMOs), e.g. plants or seeds. Genetically modified plants (or transgenic plants) are plants in which a heterologous gene has been stably integrated into the genome. The term “heterologous gene” means essentially a gene which is provided or assembled outside the plant and which, upon introduction into the nuclear genome, the chloroplast genome or the mitochondrial genome, imparts to the transformed plant novel or improved agronomical or other traits because it expresses a protein or polypeptide of interest or another gene which is present in the plant, or other genes which are present in the plant are down-regulated or switched off (for example by means of antisense technology, co-suppression technology or RNAi technology [RNA interference]). A heterologous gene that is located in the genome is also called a transgene. A transgene that is defined by its specific presence in the plant genome is called a transformation or transgenic event.

Depending on the plant species or plant cultivars, their location and growth conditions (soils, climate, vegetation period, diet), the inventive treatment may also result in superadditive (“synergistic”) effects.

For example, the following effects which exceed the effects actually to be expected are possible: reduced application rates and/or widened spectrum of activity and/or increased efficacy of the active ingredients and compositions which can be used in accordance with the invention, better plant growth, increased tolerance to high or low temperatures, increased tolerance to drought or to water or soil salinity, increased flowering performance, easier harvesting, accelerated maturation, higher harvest yields, bigger fruits, greater plant height, greener leaf color, earlier flowering, higher quality and/or a higher nutritional value of the harvested products, higher sugar concentration within the fruits, better storage stability and/or processability of the harvested products.

Plants and plant cultivars which are preferably treated in accordance with the invention include all plants which have genetic material which imparts particularly advantageous, useful traits to these plants (whether obtained by breeding and/or biotechnological means).

Examples of nematode-resistant plants are described, for example, in the following U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 and 12/497,221.

Plants that may be treated according to the invention are hybrid plants that already express the characteristics of heterosis, or hybrid effect, which results in generally higher yield, vigor, better health and resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (e.g. in maize) be produced by detasselling (i.e. the mechanical removal of the male reproductive organs or male flowers) but, more typically, male sterility is the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored.

This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar. Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate by various methods. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science, 221, 370-371), the CP4 gene of the bacterium Agrobacterium sp. (Barry et al., 1992, Curr. Topics Plant Physiol. 7, 139-145), the genes encoding a petunia EPSPS (Shah et al., 1986, Science 233, 478-481), a tomato EPSPS (Gasser et al., 1988, J. Biol. Chem. 263, 4280-4289) or an Eleusine EPSPS (WO 01/66704). It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes.

Plants which express EPSPS genes which impart glyphosate tolerance have been described. Plants which express other genes which impart glyphosate tolerance, for example decarboxylase genes, have been described.

Other herbicide-resistant plants are for example plants made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyse the reaction in which para-hydroxyphenylpyruvate (HPP) is converted to homogentisate. Plants tolerant to HPPD inhibitors can be transformed with a gene encoding a naturally-occurring resistant HPPD enzyme, or a gene encoding a mutated or chimeric HPPD enzyme, as described in WO 96/38567, WO 99/24585, WO 99/24586, WO 2009/144079, WO 2002/046387 or U.S. Pat. No. 6,768,044. Tolerance to HPPD inhibitors can also be obtained by transforming plants with genes encoding certain enzymes enabling the formation of homogentisate despite inhibition of the native HPPD enzyme by the HPPD inhibitor. Such plants are described in WO 99/34008 and WO 02/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928. In addition, plants can be made more tolerant to HPPD inhibitors by inserting into the genome thereof a gene which encodes an enzyme which metabolizes or degrades HPPD inhibitors, for example CYP450 enzymes (see WO 2007/103567 and WO 2008/150473).

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright (Weed Science 2002, 50, 700-712). The production of sulfonylurea-tolerant plants and imidazolinone-tolerant plants has been described. Further sulfonylurea- and imidazolinone-tolerant plants have also been described.

Further plants tolerant to imidazolinones and/or sulfonylureas can be obtained by induced mutagenesis, by selection in cell cultures in the presence of the herbicide or by mutation breeding (cf., for example, for soya beans U.S. Pat. No. 5,084,082, for rice WO 97/41218, for sugar beet U.S. Pat. No. 5,773,702 and WO 99/057965, for lettuce U.S. Pat. No. 5,198,599 or for sunflower WO 01/065922).

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:
a. plants which contain a transgene capable of reducing the expression and/or the activity of the poly(ADP-ribose) polymerase (PARP) gene in the plant cells or plants;
b. plants which contain a stress tolerance-enhancing transgene capable of reducing the expression and/or the activity of the PARG-encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage stability of the harvested product and/or altered properties of specific components of the harvested product such as, for example:
1) Transgenic plants which synthesize a modified starch which, in its physicochemical characteristics, in particular the amylose content or the amylose/amylopectin ratio, the degree of branching, the average chain length, the side chain distribution, the viscosity behaviour, the gelling strength, the starch granule size and/or the starch granule morphology, is changed in comparison with the synthesized starch in wild-type plant cells or plants, so that this modified starch is better suited to specific applications.
2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.
3) Transgenic plants which produce hyaluronan.
4) Transgenic plants or hybrid plants such as onions with particular properties, such as “high soluble solids content”, “low pungency” (LP) and/or “long storage” (LS).

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:
a) plants, such as cotton plants, containing an altered form of cellulose synthase genes;
b) plants, such as cotton plants, which contain an altered form of rsw2 or rsw3 homologous nucleic acids, such as cotton plants with an increased expression of sucrose phosphate synthase;
c) plants, such as cotton plants, with increased expression of sucrose synthase;
d) plants, such as cotton plants, wherein the timing of the plasmodesmatal gating at the basis of the fibre cell is altered, for example through downregulation of fibre-selective 0-1,3-glucanase;
e) plants, such as cotton plants, which have fibres with altered reactivity, for example through expression of the N-acetylglucosaminetransferase gene, including nodC, and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Plants or plant cultivars (which can be obtained by plant biotechnology methods such as genetic engineering) which may also be treated according to the invention are plants such as potatoes which are virus-resistant, for example to the potato virus Y (SY230 and SY233 events from Tecnoplant, Argentina), or which are resistant to diseases such as potato late blight (e.g. RB gene), or which exhibit reduced cold-induced sweetness (which bear the genes Nt-Inh, II-INV) or which exhibit the dwarf phenotype (A-20 oxidase gene).

Plants or plant cultivars (obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants containing a mutation imparting such altered characteristics, and include plants such as oilseed rape with retarded or reduced seed shattering.

Particularly useful transgenic plants which can be treated according to the invention are plants with transformation events or combinations of transformation events which are the subject of granted or pending petitions for nonregulated status in the USA at the Animal and Plant Health Inspection Service (APHIS) of the United States Department of Agriculture (USDA). Information relating to this is available at any time from APHIS (4700 River Road Riverdale, MD 20737, USA), for example via the website http://www.aphis.usda.gov/brs/not_reg.html. At the filing date of this application, the petitions with the following information were either granted or pending at APHIS:
Petition: Identification number of the petition. The technical description of the transformation event can be found in the specific petition document available from APHIS on the website via the petition number. These descriptions are hereby disclosed by reference.
Extension of a petition: Reference to an earlier petition for which an extension of scope or term is being requested.
Institution: Name of the person submitting the petition.
Regulated article: The plant species in question.
Transgenic phenotype: The trait imparted to the plant by the transformation event.
Transformation event or line: The name of the event(s) (sometimes also referred to as line(s)) for which nonregulated status is being requested.
APHIS documents: Various documents which have been published by APHIS with regard to the petition or can be obtained from APHIS on request.

Particularly useful transgenic plants which can be treated in accordance with the invention are plants which comprise one or more genes which code for one or more toxins, for example the transgenic plants which are sold under the following trade names: YIELD GARD® (for example maize, cotton, soybeans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned include maize varieties, cotton varieties and soya bean varieties which are available under the following trade names: Roundup Ready® (tolerance to glyphosates, for example maize, cotton, soybeans), Liberty Link® (tolerance to phosphinothricin, for example oilseed rape), IMI® (tolerance to imidazolinone) and SCS® (tolerance to sulfonylurea), for example maize. Herbicide-resistant plants (plants bred in a conventional manner for herbicide tolerance) which may be mentioned include the varieties sold under the name Clearfield® (for example maize).

The examples which follow illustrate the present invention.

### EXAMPLES

The present invention is illustrated in detail by the examples which follow, but these examples do not restrict the invention in any way.

### A. Synthesis Examples

### Methyl (2R)-2-{[4-bromo-1-(3-fluoropyridin-2-yl)-5-(6-fluoropyridin-3-yl)-1H-pyrazol-3-yl]oxy}propanoate (I-05)

### N′-(3-Fluoropyridin-2-yl)-3-(6-methylpyridin-3-yl)prop-2-yne hydrazide

To a solution of 1.27 g (6.92 mmol) of 3-(6-fluoropyridin-3-yl)prop-2-ynoic acid, 1.32 g (10.38 mmol) of 2-fluoro-6-hydrazinopyridine and 3.5 g (34.61 mmol) of triethylamine in 72 ml of THF is added dropwise 6.6 g (10.38 mmol) of a 50% propanephosphonic anhydride solution in THF, and this mixture is stirred at room temperature for one hour. For workup, H₂O is added, the organic phase is removed, and the aqueous phase is extracted repeatedly with CH₂Cl₂. The combined organic phase is dried over Na₂SO₄ and concentrated. 2.25 g (95%) of crude product is obtained, which is used without further purification for the next reaction stage.

### 1-(3-Fluoropyridin-2-yl)-5-(6-fluoropyridin-3-yl)-1H-pyrazol-3-ol

To a solution of 2.23 g (6.50 mmol) of N′-(3-fluoropyridin-2-yl)-3-(6-methylpyridin-3-yl)prop-2-yne hydrazide in 10 ml of CH₂Cl₂ and 3 ml of DMF is added 99 mg (0.52 mmol) of CuI, and the mixture is stirred at 80° C. for one hour. This is followed by removal by filtration and concentration, and purification of the crude product by column chromatography using silica gel with heptane/ethyl acetate (3:7). In this way, 0.95 g (53%) of product is obtained in solid form.

¹H-NMR (400 MHz, DMSO-d₆): δ 6.25 (bs, 1H), 7.15 (d, 1H), 7.50 (bs, 1H), 7.78 (m, 1H), 7.95 (m, 1H), 8.10 (s, 1H), 8.20 (bs, 1H).

### Methyl (2R)-2-{[1-(3-fluoropyridin-2-yl)-5-(6-fluoropyridin-3-yl)-1H-pyrazol-3-yl]oxy}propanoate

To a solution of 0.34 g (1.25 mmol) of 1-(3-fluoropyridin-2-yl)-5-(6-fluoropyridin-3-yl)-1H-pyrazol-3-ol in 10 ml of DMF are successively added 0.61 g (1.87 mmol) of Cs₂CO₃ and 0.18 g (1.50 mmol) of methyl (2S)-2-chloropropanoate, and then the mixture is stirred at 80° C. for 1 hour. Thereafter, the reaction mixture is concentrated to dryness, taken up in H₂O and extracted repeatedly with CH₂Cl₂. The organic phase is dried over Na₂SO₄ and then concentrated. Purification by column chromatography on silica gel using heptane/ethyl acetate (8.2) gives 0.4 g (87%) of product as a colourless oil.

¹H-NMR (400 MHz, CDCl₃): δ 1.65 (d, 3H), 3.79 (s, 3H), 5.25 (q, 1H), 6.14 (s, 1H), 6.89 (m, 1H), 7.35 (m, 1H), 7.50 (m, 1H), 7.68 (m, 1H), 8.10 (s, 1H), 8.25 (d, 1H).

### Methyl (2R)-2-{[4-bromo-1-(3-fluoropyridin-2-yl)-5-(6-fluoropyridin-3-yl)-1H-pyrazol-3-yl]oxy}propanoate

To a solution of 0.38 g (1.05 mmol) of methyl (2R)-2-{[1-(3-fluoropyridin-2-yl)-5-(6-fluoropyridin-3-yl)-1H-pyrazol-3-yl]oxy}propanoate in 4 ml of DMF is added 0.22 g (1.26 mmol) of N-bromosuccinimide, and the mixture is stirred at 65° C. for 1 hour. The reaction mixture is then added to H₂O and extracted repeatedly with CH₂Cl₂. The organic phase is dried over Na₂SO₄ and concentrated, and the crude product thus obtained is purified by column chromatography using silica gel with heptane/ethyl acetate (8:2). In this way, 0.45 g (95%) of product is obtained in solid form.

¹H-NMR (400 MHz, CDCl₃): δ 1.70 (d, 3H), 3.78 (s, 3H), 5.70 (q, 1H), 6.95 (m, 1H), 7.30 (m, 1H), 7.50 (m, 1H), 7.84 (m, 1H), 8.12 S, 1H), 8.20 (d, 1H).

### Methyl (2R)-2-{[4-cyano-1-(3-fluoropyridin-2-yl)-5-(6-fluoropyridin-3-yl)-1H-pyrazol-3-yl]oxy}propanoate (I-06)

Under argon, a baked-out MW vial is charged with a mixture of 0.17 g (0.40 mmol) of methyl (2R)-2-{[4-bromo-1-(3-fluoropyridin-2-yl)-5-(6-fluoropyridin-3-yl)-1H-pyrazol-3-yl]oxy}propanoate, 0.045 g (0.38 mmol) of Zn(CN)₂ and 0.046 g (0.040 mmol) of tetrakis(triphenylphosphine)palladium, and 5 ml of N,N-dimethylacetamide is added. The vessel is degassed and blanketed with argon, and the reaction mixture is stirred in a microwave at 180° C. for 40 minutes. The reaction mixture is then concentrated, taken up in H₂O and extracted repeatedly with CH₂Cl₂. The organic phase is dried over Na₂SO₄ and concentrated, and the crude product thus obtained is purified by column chromatography using silica gel with heptane/ethyl acetate (8:2). In this way, 0.082 g (52%) of product is obtained in solid form.

¹H-NMR (400 MHz, CDCl₃): δ 1.70 (d, 3H), 3.79 (s, 3H), 5.77 (q, 1H), 7.00 (m, 1H), 7.45 (m, 1H), 7.68 (m, 1H), 7.92 (m, 1H), 8.12 (s, 1H), 8.29 (d, 1H).

### Methyl (2R)-2-{[4-chloro-5-(2-fluoropyridin-4-yl)-1-(pyridin-2-yl)-1H-pyrazol-3-yl]oxy}propanoate (..) and (2R)-2-{[4-chloro-5-(2-fluoropyridin-4-yl)-1-(pyridin-2-yl)-1H-pyrazol-3-yl]oxy}propanoic acid (..)

### 5-amino-4-chloro-1-(pyridin-2-yl)-1H-pyrazol-3-ol

To a solution of 2 g (11.35 mmol) of 5-amino-1-(pyridin-2-yl)-1H-pyrazol-3-ol in 80 ml of DMF is added, in portions, 1.10 g (12.48 mmol) of N-chlorosuccinimide. The reaction mixture is stirred at 50° C. for 5 hours and then left to stand at room temperature for 12 hours. After addition of 80 ml of H₂O and 60 ml of CH₂Cl₂, the product precipitates out in solid form. Filtration and drying under reduced pressure gives 1.43 g (60%) of product in solid form.

¹H-NMR (400 MHz, DMSO-d₆): δ 7.10 (bs, 2H, NH2), 7.20 (dd, 1H), 7.55 (d, 1H), 7.90 (dd, 1H), 8.46 (d, 1H).

### Methyl (2R)-2-{[5-amino-4-chloro-1-(pyridin-2-yl)-1H-pyrazol-3-yl]oxy}propanoate

To a solution of 1.4 g (6.64 mmol) of 5-amino-4-chloro-1-(pyridin-2-yl)-1H-pyrazol-3-ol is added 3.25 g (9.98 mmol) of Cs₂CO₃, and this mixture is stirred at room temperature for 10 minutes. After addition of 1.20 g (7.97 mmol) of methyl (2S)-2-chloropropanoate, the reaction mixture is stirred at room temperature for 12 hours. After filtration, the reaction solution is added to H₂O and extracted repeatedly with CH₂Cl₂. The organic phase is dried over Na₂SO₄ and concentrated. 1.56 g (75%) of product is obtained in solid form.

¹H-NMR (400 MHz, CDCl₃): δ 1.65 (d, 3H), 3.75 (s, 3H), 5.18 (q, 1H), 6.00 (bs, 2H, NH2), 7.04 (dd, 1H), 7.63 (d, 1H), 7.72 (dd, 1H), 8.26 (d, 2H).

### Methyl (2R)-2-{[4-chloro-5-iodo-1-(pyridin-2-yl)-1H-pyrazol-3-yl]oxy}propanoate

To a solution of 0.30 g (1.01 mmol) of methyl (2R)-2-{[5-amino-4-chloro-1-(pyridin-2-yl)-1H-pyrazol-3-yl]oxy}propanoate in 6 ml of acetonitrile are successively added 1.08 g (4.04 mmol) of diiodomethane and 0.23 g (2.02 mmol) of isopentyl nitrite, and the mixture is then stirred at 50° C. for 1 hour. Thereafter, the reaction mixture is added to H₂O and extracted repeatedly with CH₂Cl₂. The organic phase is dried over Na₂SO₄ and concentrated, and the crude product is purified by column chromatography on silica gel using heptane/ethyl acetate (1:1). In this way, 0.24 g (55%) of product is obtained as an oil.

¹H-NMR (400 MHz, CDCl₃): δ 1.68 (d, 3H), 3.75 (s, 3H), 5.20 (q, 1H), 7.21 (dd, 1H), 7.60 (d, 1H), 7.70 (dd, 1H), 8.45 (d, 1H).

### (2R)-2-{[4-Chloro-5-(2-fluoropyridin-4-yl)-1-(pyridin-2-yl)-1H-pyrazol-3-yl]oxy}propanoic acid (I-21)

Under argon, a mixture of 0.18 g (0.45 mmol) of methyl (2R)-2-{[4-chloro-5-iodo-1-(pyridin-2-yl)-1H-pyrazol-3-yl]oxy}propanoate, 0.16 g (1.13 mmol) of (2-fluoropyridin-4-yl)boronic acid, 0.055 g (0.067 mmol) of PdCl₂dppf, DCM complex, and 0.44 g (1.35 mmol) of Cs₂CO₃ in 3 ml of 1,4-dioxane and 0.9 ml of H₂O is stirred in a microwave at 130° C. for 3.5 hours. Thereafter, the reaction mixture is added to a saturated aqueous NH₄Cl solution and extracted repeatedly with CH₂Cl₂. The organic phase is dried over Na₂SO₄ and concentrated, and the crude product thus obtained is purified by column chromatography using silica gel with heptane/ethyl acetate (1:1). In this way, 38 mg (21%) of the methyl ester was obtained. Concentrating the water phase and subsequent purification by column chromatography using silica gel with heptane/ethyl acetate (3:7) gives 67 mg (33%) of the carboxylic acid.

¹H-NMR (400 MHz, DMSO-d₆): δ 1.55 (d, 3H), 5.10 (q, 1H), 7.26 (m, 2H) 7.65 (d, 1H), 7.96 (m, 2H), 8.10 (d, 1H), 8.20 (s, 1H).

### Ethyl {[4-bromo-5-(2-fluoropyridin-4-yl)-1-(pyridin-2-yl)-1H-pyrazol-3-yl]oxy}acetate (I-01)

### 2-[3-(Benzyloxy)-1H-pyrazol-1-yl]pyridine

A mixture consisting of 5 g (28.70 mmol) of 3-(benzyloxy)-1H-pyrazole, 6.80 g (43.05 mmol) of 2-bromopyridine, 0.77 g (4.02 mmol) of CuI and 13.09 g (40.18 mmol) of Cs₂CO₃ in 40 ml of DMF is stirred under argon at 100° C. for 5 hours. Thereafter, the reaction mixture is filtered through kieselguhr, added to H₂O and extracted repeatedly with CH₂Cl₂. The organic phase is dried over Na₂SO₄ and concentrated, and the crude product is purified by column chromatography on silica gel using heptane/ethyl acetate (1:1). In this way, 6.65 g (92%) of product is obtained in solid form.

¹H-NMR (400 MHz, CDCl₃): δ 5.31 (s, 2H), 5.95 (s, 1H), 7.10 (dd, 1H), 7.35 (m, 3H), 7.50 (m, 2H), 7.75 (m, 1H), 7.80 (d, 1H), 8.35 (d, 1H), 8.40 (s, 1H).

### 2-[3-(Benzyloxy)-5-iodo-1H-pyrazol-1-yl]pyridine

To a solution of 31.80 mmol of LDA in 140 ml of THF is added dropwise, at −78° C. under argon, a solution of 4.70 g (18.70 mmol) of 2-[3-(benzyloxy)-1H-pyrazol-1-yl]pyridine in 130 ml THF, and this mixture is stirred at −78° C. for a further 1.5 hours. A solution of 7.6 g (30.00 mmol) of iodine in 130 ml THF is then added dropwise to the reaction mixture, and the reaction mixture is stirred for a further 2 hours at −78° C., before being allowed to come to room temperature. The reaction mixture is then added to H₂O and extracted repeatedly with CH₂Cl₂. The organic phase is dried over Na₂SO₄ and concentrated, and the crude product is purified by column chromatography on silica gel using heptane/ethyl acetate (8:2). In this way, 3.83 g (54%) of product is obtained as an oil.

¹H-NMR (400 MHz, CDCl₃): δ 5.29 (s, 2H), 6.20 (s, 1H), 7.25 (m, 1H), 7.24 (m, 1H), 7.38 (m, 2H), 7.45 (m, 1H), 7.73 (d, 1H), 8.50 (s, 1H).

### 4-[3-(Benzyloxy)-1-(pyridin-2-yl)-1H-pyrazol-5-yl]-2-fluoropyridine

To a solution of 0.50 g (1.32 mmol) of 2-[3-(benzyloxy)-5-iodo-1H-pyrazol-1-yl]pyridine in 8 ml of 1,4-dioxane are added successively under argon 0.65 g (2.91 mmol) of 2-fluoro-4-(4,4,5,5-tetramethyl-1,3,2-dioxaborolan-2-yl)pyridine, 0.86 g (2.65 mmol) of Cs₂CO₃, 13 mg (0.066 mmol) of CuI, 1 ml of H₂O and 77 mg (0.066 mmol) of tetrakis(triphenylphosphine)palladium(0), and this mixture is stirred at 70° C. for 3.5 hours and then left to stand overnight. After filtration, the reaction solution is concentrated and the crude mixture is purified by column chromatography using silica gel with heptane/ethyl acetate (7:3). In this way, 0.32 g (69%) of product is obtained in solid form.

1-H-NMR (400 MHz, CDCl3) δ 5.35 (s, 1H), 6.10 (s, 1H), 6.85 (s, 1H), 7.08 (d, 1H), 7.20 (m, 1H), 7.40 (m, 3H) 7.50 (m, 2H), 7.70 (d, 1H), 7.82, (m, 1H), 8.20 (m, 2H), 8.40 (d, 1H).

### 5-(2-Fluoropyridin-4-yl)-1-(pyridin-2-yl)-1H-pyrazol-3-ol

To a solution of 0.44 g (0.63 mmol) of 5-(2-fluoropyridin-4-yl)-1-(pyridin-2-yl)-1H-pyrazol-3-ol in 18 ml of toluene is added 37 g (324.5 mmol) of trifluoroacetic acid (dried over molecular sieve), and then the mixture is stirred at 50° C. for 3.5 hours and left to stand at room temperature overnight. Thereafter, another 3 ml of trifluoroacetic acid is added and the mixture is stirred at 50° C. for a further 3.5 hours. After filtration, the reaction solution is concentrated and the crude product is purified by column chromatography using silica gel with heptane/ethyl acetate (1:1). The mixture thus obtained contains 50% product and is used in the next reaction step without further purification.

### Ethyl {[5-(2-fluoropyridin-4-yl)-1-(pyridin-2-yl)-1H-pyrazol-3-yl]oxy}acetate

To a solution of 0.07 g (0.273 mmol) of 5-(2-fluoropyridin-4-yl)-1-(pyridin-2-yl)-1H-pyrazol-3-ol in 5.5 ml of acetonitrile is added 0.23 g (1.64 mmol) of K₂CO₃, and the mixture is stirred at room temperature for 10 minutes. After addition of 0.091 g (0.55 mmol) of ethyl bromoacetate, the reaction mixture is stirred under reflux for 5 hours and then left to stand at room temperature overnight. For workup, the mixture is concentrated to dryness, and the residue is taken up in H₂O and extracted repeatedly with CH₂Cl₂. The organic phase is dried over Na₂SO₄ and concentrated, and the crude product is purified by column chromatography on silica gel using heptane/ethyl acetate (7:3). 0.036 g (38%) of product is obtained in solid form.

¹H-NMR (400 MHz, CDCl₃): δ 1.32 (t, 3h), 4.30 (q, 2H), 4.90 (s, 2H), 6.15 (s, 1H), 6.85 (s, 1H), 7.08 (s, 1H), 7.08 (m, 1H), 7.15 (dd, 1H), 7.65 (d, 1H), 7.80 dd, 1H), 8.15 (m, 2H).

### Ethyl {[4-bromo-5-(2-fluoropyridin-4-yl)-1-(pyridin-2-yl)-1H-pyrazol-3-yl]oxy}acetate

To a solution of 0.036 g (0.105 mmol) of ethyl {[5-(2-fluoropyridin-4-yl)-1-(pyridin-2-yl)-1H-pyrazol-3-yl]oxy}acetate in 0.6 ml of CH₂Cl₂ is added 0.021 g (0.116 mmol) of N-bromosuccinimide, and the mixture is stirred at room temperature for 12 hours. This is followed by concentration to dryness, extraction of the residue by stirring with diethyl ether, and removal by filtration. The ether phase is concentrated, and the crude product thus obtained is purified using silica gel with heptane/ethyl acetate (1.1). In this way, 0.021 g (47%) of product is obtained in solid form.

¹H-NMR (400 MHz, CDCl₃): δ 1.30 (t, 3H), 4.70 (q, 2H), 4.95 (s, 2H), 6.93 (6.93 (s, 1H), 7.12 (m, 2H), 7.62 (d, 1H), 7.69 (m, 1H), 8.08 (m, 1H), 8.25 (s, 1H).

NMR Data of Selected Examples:

The ¹H NMR data of selected examples of compounds of the general formula (I) are stated in two different ways, namely (a) conventional NMR evaluation and interpretation or (b) in the form of ¹H NMR peak lists according to the method described below.

a) Conventional NMR Interpretation

### Example I-144

¹H-NMR (400 MHz, CDCl₃, δ, ppm): 8.40 (m, 1H), 8.30 (m, 1H), 8.16-8.15 (m, 1H), 7.48-7.39 (m, 2H), 7.26-7.23 (m, 1H), 5.25 (q, 1H), 3.75 (s, 3H), 1.63-1.57 (m, 4H), 0.84-0.77 (m, 4H).

### Example I-166

¹H-NMR (400 MHz, CDCl₃, δ, ppm): 8.43 (m, 1H), 8.32 (s, 1H), 8.20-8.19 (m, 1H), 7.47-7.41 (m, 2H), 7.28-7.24 (m, 1H), 5.29 (q, 1H), 1.67 (d, 3H), 1.62-1.55 (m, 1H), 0.81-0.75 (m, 4H).

### Example I-172

¹H-NMR (400 MHz, CDCl₃, δ, ppm): 8.39 (m, 1H), 8.31 (s, 1H), 8.16-8.15 (m, 1H), 7.48-7.39 (m, 2H), 7.26-7.22 (m, 1H), 5.24 (q, 1H), 4.21 (q, 2H), 1.62 (d, 3H), 1.61-1.57 (m, 1H), 1.25 (t, 3H), 0.86-0.76 (m, 4H).

b) NMR Peak List Method

The 1H NMR data of selected examples are noted in the form of 1H NMR peak lists. For each signal peak, first the δ value in ppm and then the signal intensity in round brackets are listed. The δ value—signal intensity number pairs for different signal peaks are listed with separation from one another by semicolons.

The peak list for one example therefore takes the form of:
δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in the Research Disclosure Database Number 564025.

I-01: ¹H-NMR(599.6 MHz, CDCl3):
δ = 8.5362 (1.8); 8.5343 (1.9); 8.5284 (1.9); 8.5262 (1.8); 8.4282 (0.4); 8.2542 (11.2); 8.2456 (11.3); 8.0981 (6.6); 8.0963 (6.6);
8.0901 (6.7); 8.0883 (6.4); 7.8572 (1.2); 7.8543 (1.2); 7.8442 (2.0); 7.8416 (2.0); 7.8312 (1.4); 7.8281 (1.3); 7.7984 (3.7); 7.7954
(3.6); 7.7846 (6.5); 7.7828 (6.3); 7.7723 (4.6); 7.7693 (4.2); 7.7612 (0.3); 7.6572 (0.3); 7.6435 (0.4); 7.6225 (10.6); 7.6088 (9.0);
7.3077 (1.6); 7.2995 (1.7); 7.2954 (1.7); 7.2872 (1.6); 7.2656 (10.5); 7.1614 (5.6); 7.1532 (6.0); 7.1522 (5.9); 7.1475 (8.9); 7.1446
(8.8); 7.1416 (8.7); 7.1394 (7.9); 7.1360 (7.9); 6.9597 (13.5); 5.0092 (0.4); 4.9453 (50.0); 4.9007 (1.0); 4.8887 (15.3); 4.8780 (1.6);
4.3122 (6.6); 4.3003 (19.7); 4.2884 (19.8); 4.2765 (8.7); 4.2646 (6.3); 4.2527 (6.3); 4.2408 (2.1); 1.3248 (21.4); 1.3129 (41.8);
1.3010 (21.0); 1.2948 (7.2); 1.2829 (13.2); 1.2711 (6.6); 1.2556 (0.6); −0.0001 (10.6); −0.0054 (0.4)
I-02: ¹H-NMR(400.6 MHz, CDCl3):
δ = 8.2031 (2.1); 8.2010 (1.6); 8.1989 (1.7); 8.1970 (2.1); 8.0734 (1.3); 8.0714 (1.4); 8.0688 (1.5); 8.0667 (1.3); 8.0613 (1.4);
8.0593 (1.4); 8.0567 (1.4); 8.0546 (1.3); 7.8629 (1.1); 7.8567 (1.1); 7.8440 (1.3); 7.8418 (1.4); 7.8378 (1.3); 7.8356 (1.3); 7.8229
(1.2); 7.8167 (1.2); 7.7707 (1.1); 7.7660 (1.1); 7.7523 (1.3); 7.7501 (1.6); 7.7476 (1.3); 7.7454 (1.6); 7.7317 (1.6); 7.7270 (1.6);
7.6445 (1.8); 7.6422 (3.0); 7.6399 (1.8); 7.6238 (1.3); 7.6215 (2.2); 7.6192 (1.2); 7.2610 (26.3); 7.1176 (1.4); 7.1151 (1.4); 7.1055
(1.4); 7.1029 (1.4); 7.0993 (1.4); 7.0967 (1.3); 7.0871 (1.4); 7.0845 (1.2); 6.9975 (1.6); 6.9963 (1.5); 6.9902 (1.5); 6.9888 (1.4);
6.9764 (1.4); 6.9750 (1.4); 6.9691 (1.5); 6.9677 (1.4); 4.9460 (15.8); 4.3229 (2.0); 4.3051 (6.2); 4.2873 (6.3); 4.2695 (2.0); 3.4880
(1.1); 3.4705 (1.1); 1.5506 (7.4); 1.3334 (7.7); 1.3157 (16.0); 1.2978 (7.6); 1.2263 (1.2); 1.2088 (2.4); 1.1912 (1.2); 0.0080
(1.0); −0.0002 (34.1); −0.0085 (1.0)
I-03: ¹H-NMR(400.0 MHz, CDCl3):
δ = 8.5762 (1.6); 8.5671 (1.5); 8.5643 (1.6); 8.4971 (2.3); 8.4934 (2.3); 8.2124 (1.7); 8.2010 (1.7); 7.7525 (1.3); 7.7325 (1.4);
7.4880 (0.8); 7.4674 (1.6); 7.4444 (1.0); 7.3219 (1.8); 7.3110 (1.8); 7.3023 (2.4); 7.2903 (1.7); 7.2819 (0.7); 7.2620 (9.2); 5.3215
(0.6); 5.3040 (1.8); 5.2866 (1.8); 5.2691 (0.6); 3.7703 (16.0); 2.7737 (1.9); 1.7011 (7.5); 1.6837 (7.4); −0.0002 (10.4)
I-04: ¹H-NMR(400.0 MHz, CDCl3):
δ = 8.6474 (1.0); 8.6434 (1.1); 8.6352 (1.1); 8.6312 (1.1); 8.4585 (1.4); 8.4539 (1.4); 8.2784 (1.0); 8.2689 (1.0); 7.8547 (0.7);
7.8503 (0.9); 7.8448 (0.7); 7.8347 (0.8); 7.8301 (1.0); 7.8248 (0.7); 7.5667 (0.6); 7.5630 (0.6); 7.5456 (1.0); 7.5422 (1.0); 7.5232
(0.8); 7.5196 (0.8); 7.4361 (0.8); 7.4268 (0.9); 7.4246 (0.8); 7.4153 (1.3); 7.4060 (0.6); 7.4038 (0.6); 7.3945 (0.6); 7.3885 (0.8);
7.3761 (0.8); 7.3685 (0.8); 7.3564 (0.8); 7.2617 (9.5); 5.2775 (1.7); 5.2600 (1.7); 3.7702 (16.0); 2.1270 (0.5); 2.0913 (0.9); 1.7023
(6.6); 1.6848 (6.5); −0.0002 (14.9)
I-05: ¹H-NMR(400.6 MHz, CDCl3):
δ = 8.2168 (0.7); 8.2151 (0.8); 8.2132 (0.8); 8.2115 (0.8); 8.2052 (0.7); 8.2034 (0.8); 8.2016 (0.8); 8.1999 (0.8); 8.1359 (1.1);
8.1340 (0.8); 8.1318 (0.8); 8.1297 (1.1); 7.8634 (0.6); 7.8572 (0.6); 7.8447 (0.7); 7.8423 (0.7); 7.8384 (0.7); 7.8360 (0.7); 7.8235
(0.6); 7.8172 (0.6); 7.5130 (0.6); 7.5093 (0.6); 7.4923 (0.8); 7.4888 (1.0); 7.4860 (0.7); 7.4690 (0.8); 7.4653 (0.8); 7.3390 (0.8);
7.3300 (0.8); 7.3274 (0.8); 7.3183 (1.3); 7.3094 (0.6); 7.3067 (0.6); 7.2977 (0.6); 7.2621 (21.6); 6.9586 (0.7); 6.9572 (0.8); 6.9512
(0.8); 6.9497 (0.8); 6.9374 (0.7); 6.9359 (0.8); 6.9300 (0.8); 6.9285 (0.8); 5.2887 (1.6); 5.2713 (1.6); 3.7688 (16.0); 2.0454 (0.5);
1.6986 (6.5); 1.6812 (6.5); 1.5647 (2.2); 1.2596 (0.5); 0.8818 (0.7); −0.0002 (13.4)
I-08: ¹H-NMR(400.6 MHz, d₆-DMSO):
δ = 13.0716 (0.6); 8.2690 (2.0); 8.2666 (2.7); 8.2656 (2.6); 8.2635 (2.2); 8.2573 (2.3); 8.2551 (2.9); 8.2541 (2.6); 8.2518 (2.2);
8.2214 (3.2); 8.2151 (3.5); 7.9863 (3.0); 7.9826 (2.2); 7.9804 (1.8); 7.9653 (4.1); 7.9613 (4.9); 7.9589 (2.9); 7.9578 (2.8); 7.9461
(1.8); 7.9401 (3.4); 7.9367 (2.2); 7.6052 (2.0); 7.5958 (2.4); 7.5936 (2.1); 7.5843 (3.7); 7.5750 (1.8); 7.5727 (1.9); 7.5634 (1.7);
7.2910 (2.0); 7.2897 (2.1); 7.2841 (2.2); 7.2828 (2.1); 7.2697 (2.0); 7.2684 (2.1); 7.2628 (2.1); 7.2614 (2.0); 5.7569 (1.2); 4.8568
(16.0); 3.3362 (17.9); 2.6709 (0.6); 2.5247 (1.5); 2.5200 (2.0); 2.5112 (28.2); 2.5067 (62.8); 2.5021 (89.3); 2.4975 (63.5); 2.4929
(30.3); 2.4693 (1.1); 2.3291 (0.6); 1.2472 (1.0); 0.8749 (0.5); 0.8581 (2.1); 0.8404 (0.7); 0.0080 (1.6); 0.0056 (0.5); 0.0047
(0.6); −0.0002 (52.2); −0.0050 (1.0); −0.0058 (0.9); −0.0067 (0.8); −0.0085 (1.7)
I-10: ¹H-NMR(400.6 MHz, CDCl3):
δ = 8.2697 (2.5); 8.2677 (2.6); 8.2595 (2.4); 8.2580 (2.7); 8.2560 (2.7); 8.1330 (3.5); 8.1268 (3.9); 7.8671 (1.6); 7.8608 (1.6);
7.8484 (1.9); 7.8458 (2.1); 7.8421 (2.0); 7.8397 (2.1); 7.8272 (1.8); 7.8210 (1.8); 7.5068 (1.6); 7.5031 (1.7); 7.4860 (2.3); 7.4827
(2.9); 7.4801 (2.1); 7.4630 (2.2); 7.4593 (2.2); 7.3572 (2.1); 7.3481 (2.5); 7.3455 (2.3); 7.3364 (3.7); 7.3274 (1.8); 7.3248 (1.8);
7.3158 (1.6); 7.2610 (26.2); 6.9684 (2.4); 6.9623 (2.4); 6.9611 (2.4); 6.9471 (2.4); 6.9410 (2.4); 6.9399 (2.3); 5.3734 (1.2); 5.3560
(4.6); 5.3385 (4.7); 5.3210 (1.3); 1.7447 (15.9); 1.7272 (16.0); 1.7147 (0.8); 0.8819 (0.7); 0.0079 (1.2); −0.0002 (40.7); −0.0085 (1.6)
I-11: ¹H-NMR(400.0 MHz, CDCl3):
δ = 8.1907 (1.2); 8.1888 (1.0); 8.1865 (1.0); 8.1845 (1.2); 7.8990 (1.7); 7.8916 (1.7); 7.8418 (0.6); 7.8356 (0.6); 7.8229 (0.7);
7.8207 (0.8); 7.8167 (0.7); 7.8145 (0.7); 7.8018 (0.6); 7.7956 (0.6); 7.6469 (0.7); 7.6459 (0.7); 7.6372 (0.8); 7.6362 (0.8); 7.6243
(1.0); 7.6233 (1.0); 7.6147 (1.0); 7.6138 (1.0); 7.4984 (0.8); 7.4910 (0.7); 7.4796 (0.8); 7.4759 (0.6); 7.4722 (0.8); 7.4685 (0.6);
7.4571 (0.6); 7.4497 (0.6); 7.2629 (7.7); 7.0011 (0.8); 6.9998 (0.8); 6.9937 (0.8); 6.9925 (0.8); 6.9800 (0.8); 6.9787 (0.8); 6.9726
(0.8); 6.9712 (0.8); 5.2486 (1.6); 5.2312 (1.6); 3.7972 (16.0); 2.0449 (0.7); 1.7171 (6.3); 1.6996 (6.3); 1.5757 (0.7); 1.2641 (0.8);
1.2594 (0.9); 0.8816 (1.5); 0.8638 (0.6); −0.0002 (5.4)
I-13: ¹H-NMR(599.6 MHz, CDCl3):
δ = 8.1922 (12.8); 8.1884 (13.0); 7.9613 (0.4); 7.9565 (0.4); 7.9151 (14.6); 7.9102 (14.9); 7.8328 (4.0); 7.8288 (4.0); 7.8189 (6.7);
7.8161 (6.5); 7.8063 (4.3); 7.8022 (4.1); 7.6173 (6.5); 7.6110 (6.7); 7.6022 (7.7); 7.5959 (7.6); 7.5375 (0.4); 7.5334 (0.5); 7.4536
(4.7); 7.4487 (4.7); 7.4410 (5.6); 7.4386 (5.7); 7.4363 (6.0); 7.4339 (5.0); 7.4262 (4.2); 7.4212 (3.8); 7.4115 (0.4); 7.2607 (34.4);
7.0003 (7.3); 6.9959 (7.4); 6.9862 (7.2); 6.9818 (7.2); 6.6449 (0.4); 6.6292 (0.4); 5.3183 (3.0); 5.3067 (9.8); 5.2950 (9.9); 5.2833
(3.1); 5.2697 (0.4); 5.2580 (0.3); 3.9749 (0.7); 2.2711 (1.2); 1.7606 (39.1); 1.7489 (38.9); 1.7322 (1.3); 1.4320 (8.8); 1.2550 (2.8);
0.8797 (0.4); 0.0701 (0.6); 0.0052 (2.2); −0.0001 (50.0); −0.0056 (1.8)
I-14: ¹H-NMR(400.0 MHz, d₆-DMSO):
δ = 13.1318 (0.6); 8.2740 (3.6); 8.2679 (3.6); 8.1682 (4.9); 8.1607 (5.2); 8.0299 (1.4); 8.0237 (1.4); 8.0090 (2.2); 8.0028 (2.1);
7.9891 (1.5); 7.9829 (1.5); 7.9601 (1.7); 7.9526 (1.5); 7.9395 (2.0); 7.9377 (2.3); 7.9301 (2.2); 7.9171 (2.0); 7.9095 (1.8); 7.7365
(2.8); 7.7268 (2.9); 7.7138 (2.4); 7.7042 (2.3); 7.3072 (2.3); 7.3009 (2.3); 7.2859 (2.2); 7.2797 (2.1); 4.9188 (16.0); 3.3231 (1.2);
2.5249 (0.8); 2.5202 (1.0); 2.5114 (16.8); 2.5069 (36.9); 2.5023 (51.5); 2.4978 (36.1); 2.4932 (16.1); 2.1834 (0.6); 1.3559 (4.6);
0.0080 (1.0); −0.0002 (37.2); −0.0085 (1.1)
I-15: ¹H-NMR(400.6 MHz, CDCl3):
δ = 8.4443 (1.3); 8.4376 (1.4); 8.2932 (1.5); 8.2037 (0.8); 8.2020 (0.9); 8.2002 (0.9); 8.1921 (0.9); 8.1904 (1.0); 8.1885 (1.0);
7.5266 (1.0); 7.5239 (0.9); 7.5221 (0.9); 7.5197 (1.0); 7.5152 (0.7); 7.5070 (1.0); 7.5040 (1.8); 7.5003 (1.2); 7.4973 (0.9); 7.4929
(0.7); 7.4838 (0.8); 7.4801 (0.8); 7.3419 (0.7); 7.3329 (0.8); 7.3303 (0.8); 7.3213 (1.3); 7.3123 (0.6); 7.3096 (0.7); 7.3007 (0.6);
7.2614 (30.4); 5.2864 (1.6); 5.2690 (1.6); 3.7710 (16.0); 2.0454 (0.6); 1.7015 (6.6); 1.6841 (6.6); 1.5664 (1.7); 1.2596 (0.6);
0.8820 (0.6); 0.0080 (0.6); −0.0002 (18.4); −0.0084 (0.7)
I-16: ¹H-NMR(400.6 MHz, CDCl3):
δ = 8.4532 (1.8); 8.4463 (1.9); 8.3017 (1.0); 8.2979 (1.8); 8.2940 (1.0); 8.2515 (0.8); 8.2497 (0.9); 8.2479 (0.9); 8.2461 (0.8);
8.2399 (0.8); 8.2381 (0.9); 8.2362 (0.9); 8.2345 (0.8); 7.5285 (0.8); 7.5261 (0.8); 7.5241 (0.9); 7.5219 (1.2); 7.5172 (0.8); 7.5057
(1.3); 7.5019 (1.9); 7.4994 (1.6); 7.4950 (0.8); 7.4823 (0.9); 7.4786 (0.9); 7.3640 (0.9); 7.3550 (1.0); 7.3524 (0.9); 7.3434 (1.5);
7.3343 (0.7); 7.3317 (0.7); 7.3227 (0.6); 7.2617 (36.6); 4.9588 (8.4); 3.8031 (16.0); 2.9631 (1.0); 2.7750 (0.6); 2.0454 (0.5);
1.5596 (5.9); 0.0080 (0.6); −0.0002 (21.9); −0.0084 (0.7)
I-17: ¹H-NMR(400.6 MHz, d₆-DMSO):
δ = 13.0844 (1.7); 8.6499 (5.8); 8.6430 (5.8); 8.3670 (3.0); 8.3628 (5.8); 8.3589 (2.9); 8.2464 (2.1); 8.2440 (2.8); 8.2430 (2.8);
8.2409 (2.4); 8.2348 (2.4); 8.2325 (3.0); 8.2294 (2.3); 7.9959 (1.8); 7.9924 (1.8); 7.9750 (2.1); 7.9710 (3.2); 7.9672 (2.0); 7.9498
(2.1); 7.9463 (2.0); 7.8424 (2.0); 7.8381 (2.1); 7.8355 (2.1); 7.8311 (1.9); 7.8188 (2.2); 7.8144 (2.3); 7.8119 (2.0); 7.8075 (1.9);
7.6006 (2.1); 7.5913 (2.4); 7.5890 (2.2); 7.5797 (3.8); 7.5704 (1.8); 7.5682 (2.0); 7.5588 (1.7); 5.7568 (4.2); 4.8604 (16.0); 3.3190
(34.6); 2.6745 (1.0); 2.6698 (1.5); 2.6652 (1.1); 2.6607 (0.5); 2.5405 (1.2); 2.5237 (3.1); 2.5190 (4.7); 2.5102 (79.1); 2.5056
(175.0); 2.5011 (247.3); 2.4965 (172.8); 2.4919 (78.5); 2.4742 (3.7); 2.4703 (3.4); 2.3328 (1.0); 2.3281 (1.5); 2.3235 (1.1); 0.1457
(0.7); 0.0079 (6.6); 0.0055 (1.5); −0.0002 (243.0); −0.0068 (2.5); −0.0085 (7.4); −0.0139 (0.9); −0.0155 (0.8); −0.0187
(0.6); −0.0211 (0.6); −0.0226 (0.7); −0.0258 (0.5); −0.0275 (0.9); −0.0313 (0.9); −0.0322 (0.8); −0.0344
(0.5); −0.1495 (0.7)
I-18: ¹H-NMR(400.6 MHz, CDCl3):
δ = 8.4613 (3.2); 8.4547 (3.3); 8.3036 (3.5); 8.2283 (2.4); 8.2263 (2.5); 8.2183 (2.2); 8.2166 (2.5); 8.2147 (2.4); 7.5540 (1.6);
7.5496 (1.7); 7.5471 (1.8); 7.5427 (1.6); 7.5319 (1.7); 7.5275 (1.9); 7.5250 (1.8); 7.5190 (2.2); 7.5142 (1.7); 7.4972 (2.2); 7.4939
(2.7); 7.4911 (1.9); 7.4740 (2.1); 7.4703 (2.0); 7.3554 (2.0); 7.3463 (2.3); 7.3438 (2.1); 7.3347 (3.5); 7.3257 (1.6); 7.3230 (1.7);
7.3141 (1.6); 7.2704 (1.0); 7.2697 (1.0); 7.2680 (1.1); 7.2672 (1.3); 7.2664 (1.6); 7.2606 (212.2); 7.2494 (0.5); 6.9970 (1.1);
5.3576 (1.2); 5.3401 (4.7); 5.3226 (4.8); 5.3052 (1.2); 5.3002 (0.9); 2.7754 (0.7); 1.7488 (16.0); 1.7313 (15.9); 1.7142 (0.6);
1.2425 (1.2); 1.2390 (1.2); 1.2222 (0.5); 0.0080 (3.7); 0.0056 (1.1); 0.0047 (1.5); −0.0002 (126.8); −0.0058 (1.4); −0.0066
(1.2); −0.0084 (3.3); −0.0115 (0.5)
I-100: ¹H-NMR(400.6 MHz, CDCl3):
δ = 8.2102 (2.1); 8.2062 (1.7); 8.2040 (2.2); 8.0824 (1.2); 8.0804 (1.4); 8.0778 (1.4); 8.0757 (1.4); 8.0703 (1.3); 8.0683 (1.4);
8.0656 (1.4); 8.0636 (1.3); 7.8669 (1.2); 7.8607 (1.1); 7.8480 (1.3); 7.8458 (1.4); 7.8419 (1.3); 7.8396 (1.4); 7.8269 (1.2); 7.8207
(1.2); 7.7753 (1.1); 7.7705 (1.1); 7.7568 (1.3); 7.7546 (1.7); 7.7521 (1.4); 7.7499 (1.6); 7.7362 (1.6); 7.7315 (1.6); 7.6443 (1.7);
7.6420 (3.0); 7.6397 (1.8); 7.6236 (1.2); 7.6213 (2.1); 7.6191 (1.3); 7.2606 (32.0); 7.1228 (1.4); 7.1203 (1.4); 7.1107 (1.3); 7.1081
(1.4); 7.1045 (1.4); 7.1019 (1.3); 7.0923 (1.3); 7.0897 (1.3); 6.9995 (1.5); 6.9981 (1.6); 6.9921 (1.5); 6.9907 (1.5); 6.9784 (1.4);
6.9770 (1.5); 6.9710 (1.4); 6.9696 (1.5); 4.9473 (16.0); 4.3249 (2.0); 4.3071 (6.3); 4.2893 (6.4); 4.2715 (2.1); 1.5495 (3.4); 1.3346
(7.8); 1.3168 (16.0); 1.2989 (7.6); 0.0080 (1.3); −0.0002 (47.6); −0.0085 (1.4)
I-101: ¹H-NMR(400.0 MHz, CDCl3):
δ = 8.1754 (2.3); 8.1693 (2.3); 8.0295 (1.4); 8.0275 (1.5); 8.0249 (1.6); 8.0229 (1.4); 8.0174 (1.4); 8.0154 (1.5); 8.0127 (1.5);
8.0107 (1.4); 7.8344 (1.2); 7.8282 (1.1); 7.8153 (1.4); 7.8134 (1.5); 7.8091 (1.4); 7.8072 (1.4); 7.7943 (1.2); 7.7881 (1.2); 7.7470
(1.1); 7.7422 (1.0); 7.7285 (1.2); 7.7262 (1.7); 7.7239 (1.3); 7.7215 (1.6); 7.7079 (1.7); 7.7031 (1.6); 7.6383 (1.8); 7.6361 (3.3);
7.6339 (2.0); 7.6177 (1.3); 7.6154 (2.2); 7.6131 (1.2); 7.2615 (16.8); 7.0810 (1.4); 7.0785 (1.5); 7.0689 (1.4); 7.0663 (1.5); 7.0628
(1.5); 7.0601 (1.4); 7.0506 (1.4); 7.0480 (1.3); 6.9933 (1.6); 6.9871 (1.6); 6.9721 (1.6); 6.9660 (1.5); 5.2437 (0.9); 5.2262 (3.1);
5.2088 (3.2); 5.1914 (0.9); 4.2901 (1.0); 4.2831 (1.1); 4.2722 (3.1); 4.2654 (3.2); 4.2544 (3.2); 4.2477 (3.1); 4.2366 (1.1); 4.2300
(1.0); 1.7114 (11.9); 1.6939 (11.9); 1.5609 (8.8); 1.3049 (7.9); 1.2871 (16.0); 1.2692 (8.6); 0.8987 (1.0); 0.8818 (3.6); 0.8641 (1.4);
0.0079 (0.5); −0.0002 (19.0); −0.0085 (0.6)
I-102: ¹H-NMR(400.6 MHz, CDCl3):
δ = 8.1982 (2.3); 8.1921 (2.4); 8.0465 (1.4); 8.0445 (1.5); 8.0419 (1.6); 8.0399 (1.5); 8.0344 (1.5); 8.0324 (1.6); 8.0298 (1.6);
8.0277 (1.5); 7.8583 (1.2); 7.8521 (1.2); 7.8393 (1.4); 7.8372 (1.5); 7.8332 (1.4); 7.8311 (1.4); 7.8183 (1.3); 7.8121 (1.2); 7.7579
(1.1); 7.7531 (1.1); 7.7395 (1.3); 7.7372 (1.8); 7.7348 (1.4); 7.7325 (1.7); 7.7189 (1.7); 7.7141 (1.7); 7.6375 (1.8); 7.6354 (3.1);
7.6332 (1.9); 7.6169 (1.3); 7.6147 (2.2); 7.6125 (1.2); 7.2626 (23.6); 7.0980 (1.5); 7.0954 (1.5); 7.0859 (1.5); 7.0833 (1.6); 7.0797
(1.6); 7.0771 (1.5); 7.0675 (1.5); 7.0650 (1.4); 6.9934 (1.6); 6.9920 (1.6); 6.9861 (1.6); 6.9847 (1.6); 6.9722 (1.5); 6.9709 (1.6);
6.9650 (1.6); 6.9636 (1.5); 5.2555 (0.9); 5.2381 (3.2); 5.2207 (3.3); 5.2033 (0.9); 4.2935 (1.0); 4.2864 (1.1); 4.2757 (3.1); 4.2687
(3.1); 4.2578 (3.2); 4.2510 (3.1); 4.2401 (1.1); 4.2333 (1.0); 3.5060 (1.9); 3.4885 (6.0); 3.4710 (6.1); 3.4535 (2.0); 1.7152 (12.3);
1.6978 (12.2); 1.5835 (6.5); 1.3055 (7.7); 1.2877 (16.0); 1.2699 (7.7); 1.2263 (6.4); 1.2088 (12.8); 1.1912 (6.2); 0.8820
(0.5); −0.0002 (11.4)
I-103: ¹H-NMR(400.6 MHz, d₆-DMSO):
δ = 8.2163 (3.4); 8.2101 (3.6); 8.0964 (2.2); 8.0944 (2.4); 8.0917 (2.7); 8.0897 (2.5); 8.0843 (2.4); 8.0823 (2.6); 8.0796 (2.7);
8.0776 (2.4); 7.9897 (1.6); 7.9834 (1.6); 7.9723 (2.5); 7.9680 (3.6); 7.9637 (2.2); 7.9624 (2.3); 7.9538 (2.6); 7.9518 (2.9); 7.9491
(4.0); 7.9472 (3.0); 7.9427 (1.8); 7.9333 (2.6); 7.9285 (2.4); 7.6686 (2.6); 7.6664 (4.8); 7.6642 (2.8); 7.6480 (2.4); 7.6459 (4.3);
7.6436 (2.4); 7.2933 (2.3); 7.2876 (2.3); 7.2719 (2.4); 7.2693 (3.1); 7.2667 (4.5); 7.2572 (2.3); 7.2547 (2.5); 7.2508 (2.5); 7.2484
(2.2); 7.2387 (2.3); 7.2362 (2.3); 4.8941 (16.0); 4.0382 (0.8); 4.0204 (0.8); 3.3370 (9.0); 2.5256 (0.8); 2.5209 (1.1); 2.5122 (15.4);
2.5076 (34.1); 2.5030 (48.2); 2.4984 (34.1); 2.4939 (15.6); 1.9891 (3.6); 1.9100 (4.0); 1.3559 (1.3); 1.1924 (1.0); 1.1747 (2.2);
1.1569 (1.0)
I-104: ¹H-NMR(400.6 MHz, CDCl3):
δ = 8.5475 (2.6); 8.5406 (2.6); 8.3448 (1.5); 8.3411 (2.6); 8.3375 (1.5); 8.1952 (1.2); 8.1930 (1.3); 8.1907 (1.4); 8.1885 (1.3);
8.1831 (1.2); 8.1807 (1.4); 8.1788 (1.4); 8.1764 (1.3); 7.9271 (0.7); 7.9225 (0.7); 7.9096 (0.6); 7.9066 (1.9); 7.9021 (1.9); 7.8892
(2.3); 7.8848 (2.3); 7.8811 (2.1); 7.8788 (2.4); 7.8774 (2.7); 7.8749 (2.4); 7.8606 (0.8); 7.8584 (0.9); 7.8570 (0.6); 7.5385 (1.2);
7.5341 (1.3); 7.5316 (1.3); 7.5272 (1.2); 7.5167 (1.3); 7.5123 (1.4); 7.5098 (1.3); 7.5054 (1.2); 7.3327 (1.4); 7.3289 (1.4); 7.3206
(1.4); 7.3166 (1.5); 7.3153 (1.6); 7.3115 (1.3); 7.3033 (1.4); 7.2993 (1.3); 7.2647 (10.0); 5.3012 (13.1); 4.4380 (15.8); 4.2762 (2.1);
4.2583 (6.8); 4.2405 (6.8); 4.2226 (2.2); 1.6028 (2.1); 1.2834 (7.6); 1.2656 (16.0); 1.2592 (0.6); 1.2568 (0.6); 1.2551 (0.6); 1.2477
(7.4); 0.0705 (3.1); −0.0002 (10.6)
I-105: ¹H-NMR(400.0 MHz, CDCl3):
δ = 8.1519 (2.3); 8.1459 (2.3); 8.0663 (1.6); 8.0636 (1.6); 8.0543 (1.6); 8.0514 (1.6); 7.8464 (0.9); 7.8403 (0.8); 7.8259 (1.3);
7.8202 (1.2); 7.8067 (0.9); 7.8006 (0.9); 7.7838 (1.1); 7.7790 (1.2); 7.7633 (1.8); 7.7586 (1.7); 7.7446 (1.6); 7.7399 (1.5); 7.6305
(3.0); 7.6099 (2.3); 7.2608 (31.2); 7.1562 (1.4); 7.1537 (1.4); 7.1440 (1.5); 7.1416 (1.5); 7.1377 (1.5); 7.1351 (1.4); 7.1255 (1.4);
7.1230 (1.3); 6.9861 (1.7); 6.9788 (1.7); 6.9659 (1.7); 6.9579 (1.6); 5.2999 (5.7); 4.9538 (16.0); 4.9476 (1.4); 4.7851 (0.6); 4.3166
(2.1); 4.2987 (6.4); 4.2808 (6.6); 4.2686 (0.7); 4.2630 (2.2); 3.9012 (0.8); 1.5454 (11.2); 1.3279 (7.7); 1.3204 (1.2); 1.3101 (15.6);
1.3025 (1.0); 1.2991 (1.2); 1.2923 (7.6); 1.2812 (0.6); 1.2552 (0.8); 0.0080 (1.3); −0.0002 (42.0); −0.0085 (1.4)
I-106: ¹H-NMR(400.6 MHz, CDCl3):
δ = 8.5338 (3.0); 8.5269 (3.1); 8.3349 (1.6); 8.3312 (3.0); 8.3276 (1.6); 8.1816 (1.2); 8.1794 (1.3); 8.1771 (1.4); 8.1749 (1.3);
8.1695 (1.3); 8.1672 (1.4); 8.1651 (1.4); 8.1628 (1.3); 7.9156 (0.8); 7.9110 (0.8); 7.8979 (0.7); 7.8951 (1.9); 7.8906 (1.8); 7.8775
(2.2); 7.8731 (2.2); 7.8668 (2.0); 7.8645 (2.4); 7.8633 (2.7); 7.8609 (2.3); 7.8463 (0.9); 7.8440 (1.0); 7.8428 (0.8); 7.8404 (0.6);
7.5133 (1.3); 7.5089 (1.3); 7.5064 (1.4); 7.5020 (1.2); 7.4913 (1.3); 7.4869 (1.4); 7.4845 (1.3); 7.4801 (1.3); 7.3059 (1.4); 7.3022
(1.4); 7.2937 (1.3); 7.2900 (1.4); 7.2883 (1.6); 7.2846 (1.3); 7.2762 (1.4); 7.2725 (1.4); 7.2626 (22.8); 5.3006 (9.1); 4.4170 (16.0);
4.2811 (1.7); 4.2633 (5.5); 4.2455 (5.8); 4.2277 (1.9); 1.5794 (5.8); 1.2966 (7.4); 1.2788 (15.6); 1.2609 (7.4); 0.0696 (3.7); 0.0080
(0.6); −0.0002 (24.8); −0.0085 (0.7)
I-107: ¹H-NMR(400.0 MHz, CDCl3):
δ = 8.1455 (1.9); 8.1395 (1.9); 8.0421 (1.2); 8.0403 (1.3); 8.0376 (1.4); 8.0356 (1.2); 8.0301 (1.3); 8.0281 (1.4); 8.0254 (1.4);
8.0235 (1.3); 7.8393 (0.8); 7.8333 (0.7); 7.8187 (1.1); 7.8131 (1.1); 7.7993 (0.8); 7.7934 (0.8); 7.7702 (1.0); 7.7654 (1.0); 7.7516
(1.2); 7.7497 (1.5); 7.7469 (1.3); 7.7450 (1.4); 7.7311 (1.4); 7.7263 (1.4); 7.6239 (1.6); 7.6217 (2.8); 7.6011 (2.0); 7.2609 (25.4);
7.1369 (1.3); 7.1345 (1.3); 7.1248 (1.3); 7.1223 (1.3); 7.1184 (1.2); 7.1159 (1.1); 7.1063 (1.2); 7.1038 (1.2); 6.9814 (1.4); 6.9741
(1.4); 6.9603 (1.4); 6.9542 (1.3); 5.2716 (0.7); 5.2543 (2.5); 5.2369 (2.6); 5.2195 (0.8); 4.2856 (1.0); 4.2817 (1.0); 4.2678 (3.0);
4.2639 (3.0); 4.2499 (3.1); 4.2462 (2.9); 4.2320 (1.0); 4.2284 (1.0); 1.7113 (0.6); 1.6999 (10.5); 1.6942 (1.0); 1.6825 (10.3);
1.5508 (16.0); 1.3047 (0.6); 1.2986 (6.5); 1.2870 (1.4); 1.2808 (13.5); 1.2693 (1.3); 1.2630 (6.8); 0.8819 (0.6); 0.0080
(0.8); −0.0002 (28.3); −0.0085 (0.8)
I-108: ¹H-NMR(400.6 MHz, CDCl3):
δ = 8.2005 (1.1); 8.1943 (1.2); 8.0860 (0.6); 8.0750 (0.6); 7.8559 (0.6); 7.8496 (0.6); 7.8369 (0.7); 7.8348 (0.8); 7.8308 (0.7);
7.8286 (0.7); 7.8158 (0.6); 7.8097 (0.6); 7.7536 (0.6); 7.2606 (39.7); 6.9925 (0.7); 6.9853 (0.7); 6.9715 (0.7); 6.9642 (0.7); 5.3003
(5.3); 5.1175 (1.3); 5.0434 (0.9); 3.7107 (2.3); 3.6798 (1.6); 3.1571 (0.6); 2.0455 (1.2); 1.5446 (16.0); 1.2596 (0.9); 0.0080 (1.6);
0.0058 (0.5); 0.0050 (0.5); −0.0002 (64.7); −0.0085 (2.0)
I-109: ¹H-NMR(400.6 MHz, CDCl3):
δ = 8.2018 (2.0); 8.2000 (2.1); 8.1978 (1.9); 8.1958 (2.2); 8.0848 (1.5); 8.0828 (1.6); 8.0801 (1.7); 8.0781 (1.5); 8.0726 (1.6);
8.0707 (1.6); 8.0680 (1.7); 8.0660 (1.4); 7.8572 (0.6); 7.8540 (0.7); 7.8510 (0.7); 7.8479 (0.7); 7.8381 (0.8); 7.8356 (1.2); 7.8326
(1.3); 7.8296 (1.2); 7.8268 (0.8); 7.8172 (0.7); 7.8140 (0.7); 7.8110 (0.7); 7.8079 (0.7); 7.7792 (0.8); 7.7745 (0.8); 7.7588 (1.5);
7.7574 (1.4); 7.7541 (1.4); 7.7401 (1.2); 7.7353 (1.2); 7.6558 (1.0); 7.6536 (1.6); 7.6513 (1.0); 7.6418 (1.1); 7.6396 (1.7); 7.6373
(1.1); 7.6353 (0.9); 7.6330 (1.2); 7.6307 (0.7); 7.6212 (0.8); 7.6190 (1.2); 7.6167 (0.7); 7.2620 (23.2); 7.1218 (1.6); 7.1192 (1.6);
7.1096 (1.6); 7.1070 (1.6); 7.1034 (1.6); 7.1008 (1.4); 7.0913 (1.5); 7.0887 (1.4); 6.9927 (1.6); 6.9853 (1.7); 6.9715 (1.6); 6.9642
(1.6); 5.3004 (13.5); 4.9833 (5.7); 4.9754 (3.7); 4.9721 (3.3); 3.8745 (1.3); 3.8643 (1.2); 3.8569 (1.3); 3.8449 (1.7); 3.8291 (1.1);
3.8229 (1.3); 3.8130 (1.2); 3.7606 (0.6); 3.7543 (0.9); 3.7428 (15.2); 3.7298 (0.6); 3.7195 (16.0); 3.6631 (0.7); 3.6381 (0.5);
3.5889 (0.7); 3.5588 (0.5); 3.2273 (0.6); 3.2088 (0.9); 3.1904 (0.6); 3.1157 (0.6); 3.0984 (0.8); 3.0796 (0.6); 2.3251 (0.8); 2.3073
(1.0); 2.2893 (0.9); 2.2743 (0.7); 2.2219 (0.6); 2.2033 (1.1); 2.1895 (0.7); 2.1842 (1.3); 2.1646 (1.0); 1.5761 (7.2); 0.0079
(1.0); −0.0002 (36.5); −0.0085 (1.0)
I-110: ¹H-NMR(400.6 MHz, CDCl3):
δ = 8.2024 (1.8); 8.1966 (2.2); 8.0706 (1.5); 8.0588 (1.5); 7.8533 (0.7); 7.8472 (0.8); 7.8324 (1.2); 7.8281 (1.3); 7.8133 (0.8);
7.8072 (0.9); 7.7781 (0.7); 7.7736 (0.7); 7.7573 (1.5); 7.7530 (1.4); 7.7394 (1.3); 7.7349 (1.3); 7.7063 (2.2); 7.6858 (1.1); 7.6621
(0.5); 7.2627 (8.3); 7.1150 (1.0); 7.1124 (1.0); 7.1027 (1.2); 7.1000 (1.3); 7.0973 (1.2); 7.0944 (1.0); 7.0850 (0.9); 7.0822 (0.8);
6.9884 (1.3); 6.9817 (1.3); 6.9675 (1.3); 6.9606 (1.2); 5.3005 (6.3); 5.0258 (8.2); 4.9364 (0.9); 4.9231 (0.8); 4.6059 (0.8); 4.5970
(1.0); 4.5859 (0.9); 4.5759 (0.9); 3.7904 (0.9); 3.7779 (0.8); 3.7717 (0.8); 3.7582 (4.0); 3.7168 (16.0); 3.7011 (1.2); 3.6833 (1.2);
3.6659 (0.6); 3.6588 (0.8); 2.2218 (0.6); 2.2096 (0.7); 2.1958 (0.6); 2.1891 (0.6); 2.1786 (0.7); 2.1666 (0.5); 2.1511 (0.6); 2.1353
(0.6); 2.0640 (0.9); 2.0453 (2.7); 2.0332 (1.1); 2.0200 (0.8); 1.5923 (3.1); 1.2773 (0.6); 1.2594 (1.4); 1.2416 (0.6); −0.0002 (12.8)
I-111: ¹H-NMR(400.6 MHz, d₆-DMSO):
δ = 8.2464 (2.3); 8.2421 (2.0); 8.2403 (2.3); 8.1035 (1.2); 8.1015 (1.4); 8.0988 (1.8); 8.0968 (1.8); 8.0943 (0.7); 8.0915 (1.5);
8.0894 (1.5); 8.0867 (1.8); 8.0847 (1.8); 8.0183 (0.9); 8.0122 (0.9); 7.9972 (1.5); 7.9924 (1.2); 7.9910 (1.2); 7.9777 (1.1); 7.9731
(1.5); 7.9716 (1.2); 7.9685 (1.1); 7.9584 (0.5); 7.9546 (1.6); 7.9526 (1.6); 7.9499 (1.3); 7.9479 (1.4); 7.9341 (1.5); 7.9293 (1.3);
7.7515 (1.4); 7.7492 (2.6); 7.7470 (1.6); 7.7309 (1.3); 7.7287 (2.3); 7.7264 (1.3); 7.7059 (0.8); 7.6853 (0.7); 7.3033 (1.6); 7.2975
(1.6); 7.2836 (2.7); 7.2814 (2.8); 7.2764 (1.7); 7.2717 (1.4); 7.2691 (1.6); 7.2654 (1.6); 7.2627 (1.5); 7.2596 (0.5); 7.2532 (1.3);
7.2505 (1.5); 5.0709 (4.1); 5.0654 (3.6); 5.0352 (0.7); 4.8073 (0.6); 4.7709 (0.5); 4.3088 (1.0); 4.2987 (1.2); 4.2870 (1.2); 4.2770
(1.0); 4.0557 (1.1); 4.0379 (3.3); 4.0202 (3.4); 4.0024 (1.1); 3.6395 (0.7); 3.6239 (1.3); 3.6074 (0.9); 3.6012 (1.8); 3.5842 (0.8);
3.5771 (0.6); 3.3227 (5.1); 2.5411 (1.3); 2.5243 (0.8); 2.5197 (1.1); 2.5109 (17.0); 2.5063 (37.8); 2.5017 (53.3); 2.4972 (37.4);
2.4926 (16.9); 2.1946 (0.5); 2.1835 (0.8); 2.1731 (0.8); 2.1634 (0.8); 2.1532 (0.5); 2.1445 (0.7); 1.9888 (16.0); 1.9679 (1.7);
1.9506 (1.2); 1.9334 (0.5); 1.9090 (1.0); 1.9045 (0.6); 1.8858 (0.6); 1.8753 (0.6); 1.8717 (0.5); 1.7593 (0.6); 1.3555 (3.5); 1.2352
(1.2); 1.1922 (4.6); 1.1745 (9.3); 1.1567 (4.4); 0.0080 (1.0); −0.0002 (35.3); −0.0085 (1.0)
I-112: ¹H-NMR(400.6 MHz, CDCl3):
δ = 8.2026 (1.9); 8.2004 (1.5); 8.1984 (1.6); 8.1964 (2.0); 8.1944 (1.4); 8.0863 (1.2); 8.0843 (1.4); 8.0816 (1.5); 8.0795 (1.4);
8.0742 (1.4); 8.0721 (1.5); 8.0695 (1.5); 8.0674 (1.4); 7.8561 (1.1); 7.8499 (1.1); 7.8372 (1.3); 7.8350 (1.4); 7.8310 (1.3); 7.8288
(1.3); 7.8161 (1.2); 7.8099 (1.2); 7.7840 (1.1); 7.7792 (1.1); 7.7655 (1.3); 7.7634 (1.6); 7.7608 (1.3); 7.7586 (1.6); 7.7449 (1.7);
7.7402 (1.6); 7.6485 (1.6); 7.6462 (3.0); 7.6439 (1.8); 7.6279 (1.3); 7.6256 (2.2); 7.6232 (1.2); 7.2623 (19.3); 7.1215 (1.4); 7.1189
(1.4); 7.1093 (1.4); 7.1068 (1.5); 7.1031 (1.5); 7.1005 (1.4); 7.0909 (1.4); 7.0883 (1.3); 6.9928 (1.4); 6.9913 (1.4); 6.9855 (1.4);
6.9839 (1.4); 6.9717 (1.4); 6.9701 (1.4); 6.9643 (1.4); 6.9628 (1.4); 5.3003 (14.6); 5.0555 (2.9); 5.0406 (3.0); 4.1890 (2.0); 4.1712
(6.4); 4.1533 (6.5); 4.1355 (2.0); 3.2242 (0.5); 2.9432 (0.5); 2.5978 (0.6); 2.5876 (1.1); 2.5775 (0.6); 2.5612 (0.6); 2.0453 (1.0);
1.9937 (0.8); 1.9686 (0.5); 1.5773 (3.7); 1.2823 (7.4); 1.2774 (0.6); 1.2645 (16.0); 1.2596 (1.3); 1.2467 (7.2); 1.2418 (0.6); 0.0079
(0.8); −0.0002 (30.9); −0.0028 (1.8); −0.0043 (0.9); −0.0052 (0.7); −0.0060 (0.6); −0.0085 (1.0)
I-113: ¹H-NMR(400.6 MHz, CDCl3):
δ = 8.3320 (2.4); 8.3303 (2.5); 8.3259 (2.5); 8.3242 (2.4); 8.1160 (1.1); 8.1138 (1.2); 8.1116 (1.4); 8.1093 (1.2); 8.1039 (1.2);
8.1012 (1.4); 8.0999 (1.5); 8.0972 (1.2); 7.8253 (0.6); 7.8208 (0.5); 7.8047 (1.9); 7.8002 (1.8); 7.7879 (2.3); 7.7834 (4.0); 7.7811
(2.7); 7.7785 (2.3); 7.7647 (0.7); 7.7625 (0.7); 7.7028 (2.4); 7.6967 (2.3); 7.6822 (2.7); 7.6761 (2.6); 7.3929 (3.0); 7.3912 (3.0);
7.3723 (2.6); 7.3706 (2.6); 7.2633 (6.3); 7.1806 (1.2); 7.1763 (1.2); 7.1685 (1.2); 7.1639 (2.3); 7.1595 (1.2); 7.1520 (1.2); 7.1474
(1.1); 5.3001 (3.1); 4.2643 (2.0); 4.2465 (6.2); 4.2287 (6.2); 4.2109 (2.0); 3.9398 (16.0); 1.5827 (5.8); 1.3003 (7.2); 1.2824 (14.6);
1.2646 (7.0); 0.0705 (1.7); −0.0002 (8.3)
I-114: ¹H-NMR(400.6 MHz, CDCl3):
δ = 8.2039 (1.4); 8.2017 (1.3); 8.1979 (1.8); 8.0764 (1.2); 8.0743 (1.3); 8.0717 (1.4); 8.0697 (1.3); 8.0643 (1.3); 8.0622 (1.4);
8.0596 (1.4); 8.0575 (1.3); 7.8566 (0.7); 7.8503 (0.8); 7.8375 (0.9); 7.8355 (0.9); 7.8312 (1.0); 7.8294 (1.1); 7.8166 (0.8); 7.8103
(0.9); 7.7791 (0.6); 7.7745 (0.6); 7.7610 (1.0); 7.7585 (1.4); 7.7539 (1.1); 7.7405 (1.3); 7.7358 (1.2); 7.7068 (1.2); 7.7045 (2.0);
7.7021 (1.4); 7.6863 (0.8); 7.6840 (1.1); 7.6813 (0.9); 7.6785 (0.6); 7.2619 (16.5); 7.1151 (0.9); 7.1121 (0.9); 7.1029 (1.1); 7.1000
(1.1); 7.0970 (1.0); 7.0941 (0.9); 7.0849 (0.9); 7.0820 (0.8); 6.9900 (1.2); 6.9827 (1.2); 6.9688 (1.2); 6.9615 (1.2); 5.3003 (8.7);
5.1281 (8.8); 5.0156 (1.9); 4.2225 (1.8); 4.1929 (7.5); 3.7930 (3.6); 3.7375 (16.0); 3.1866 (15.4); 3.0605 (3.1); 2.0453 (2.0);
1.5735 (5.7); 1.2773 (0.6); 1.2594 (1.4); 1.2562 (0.6); 1.2416 (0.6); 0.0079 (0.7); −0.0002 (25.4); −0.0085 (0.9)
I-115: ¹H-NMR(400.0 MHz, CDCl3):
δ = 9.2263 (4.1); 8.8053 (0.6); 8.7652 (10.3); 8.0695 (0.9); 8.0576 (0.9); 7.8596 (0.6); 7.8423 (1.4); 7.8397 (1.8); 7.8366 (1.4);
7.8280 (1.3); 7.8235 (1.1); 7.8104 (1.1); 7.8059 (1.0); 7.2603 (68.5); 7.1733 (0.8); 7.1697 (0.8); 7.1612 (0.8); 7.1577 (0.9); 7.1523
(0.7); 7.1435 (0.8); 7.1401 (0.7); 4.2757 (1.2); 4.2579 (3.9); 4.2400 (3.8); 4.2222 (1.3); 3.9539 (9.9); 1.7397 (2.8); 1.5435 (16.0);
1.3115 (4.6); 1.2936 (9.2); 1.2758 (4.4); 1.2557 (1.0); 0.0080 (2.6); −0.0002 (93.7); −0.0085 (2.6)
I-116: ¹H-NMR(400.6 MHz, CDCl3):
δ = 8.2102 (1.2); 8.2081 (0.9); 8.2041 (1.2); 8.0726 (0.7); 8.0706 (0.8); 8.0679 (0.8); 8.0659 (0.8); 8.0605 (0.8); 8.0585 (0.8);
8.0558 (0.9); 8.0538 (0.8); 7.8670 (0.6); 7.8608 (0.6); 7.8480 (0.7); 7.8459 (0.8); 7.8418 (0.8); 7.8397 (0.8); 7.8270 (0.7); 7.8208
(0.7); 7.7845 (0.6); 7.7797 (0.6); 7.7660 (0.7); 7.7638 (0.9); 7.7613 (0.8); 7.7591 (0.9); 7.7455 (0.9); 7.7407 (0.9); 7.6608 (1.0);
7.6586 (1.7); 7.6563 (1.0); 7.6402 (0.7); 7.6380 (1.2); 7.6357 (0.7); 7.2622 (6.1); 7.1220 (0.8); 7.1194 (0.8); 7.1099 (0.8); 7.1073
(0.8); 7.1036 (0.8); 7.1011 (0.8); 7.0915 (0.8); 7.0889 (0.7); 6.9978 (0.9); 6.9965 (0.8); 6.9904 (0.9); 6.9892 (0.8); 6.9766 (0.8);
6.9754 (0.8); 6.9693 (0.9); 6.9680 (0.8); 4.9481 (8.5); 4.5227 (2.0); 4.5070 (4.3); 4.4913 (2.2); 3.6845 (16.0); 2.7182 (2.0); 2.7026
(4.1); 2.6869 (2.0); 1.5648 (1.0); −0.0002 (9.7)
I-117: ¹H-NMR(400.6 MHz, CDCl3):
δ = 8.2124 (1.4); 8.2063 (1.4); 8.0812 (0.9); 8.0784 (1.0); 8.0691 (1.0); 8.0663 (1.0); 7.8607 (0.6); 7.8545 (0.6); 7.8416 (0.8);
7.8396 (0.9); 7.8355 (0.8); 7.8335 (0.8); 7.8207 (0.7); 7.8146 (0.7); 7.8064 (0.6); 7.8017 (0.6); 7.7858 (1.0); 7.7833 (0.9); 7.7812
(1.0); 7.7674 (1.0); 7.7627 (0.9); 7.6988 (1.9); 7.6782 (1.2); 7.2612 (15.7); 7.1464 (0.8); 7.1440 (0.8); 7.1343 (0.8); 7.1318 (0.9);
7.1282 (0.9); 7.1257 (0.8); 7.1160 (0.9); 7.1135 (0.9); 7.0077 (1.0); 7.0005 (1.0); 6.9867 (1.0); 6.9794 (1.0); 4.8751 (8.4); 3.6809
(16.0); 3.6684 (2.4); 3.6532 (2.4); 3.6378 (1.0); 2.6295 (1.9); 2.6142 (3.0); 2.5992 (1.9); 1.5551 (6.5); 1.2556 (0.7); 0.0078
(0.9); −0.0002 (25.1); −0.0085 (1.0)
I-118: ¹H-NMR(400.6 MHz, d₆-DMSO):
δ = 12.6501 (1.0); 8.4673 (1.2); 8.4527 (2.6); 8.4379 (1.2); 8.2656 (3.8); 8.2614 (3.0); 8.2594 (3.9); 8.1151 (2.5); 8.1131 (2.8);
8.1105 (3.2); 8.1084 (2.9); 8.1031 (2.7); 8.1010 (3.1); 8.0984 (3.1); 8.0963 (2.8); 8.0331 (1.8); 8.0269 (1.8); 8.0135 (2.3); 8.0119
(2.5); 8.0073 (2.2); 8.0057 (2.4); 7.9924 (2.0); 7.9861 (2.0); 7.9818 (2.4); 7.9770 (2.2); 7.9633 (2.7); 7.9611 (3.0); 7.9586 (2.5);
7.9564 (2.9); 7.9427 (3.1); 7.9379 (2.9); 7.7796 (3.1); 7.7773 (5.7); 7.7750 (3.2); 7.7589 (2.7); 7.7567 (4.8); 7.7544 (2.6); 7.3128
(2.4); 7.3115 (2.6); 7.3059 (2.6); 7.3046 (2.5); 7.2988 (3.0); 7.2963 (3.0); 7.2916 (2.5); 7.2902 (2.8); 7.2866 (3.3); 7.2843 (5.1);
7.2804 (3.1); 7.2779 (2.6); 7.2683 (2.8); 7.2657 (2.7); 4.8733 (16.0); 3.8544 (7.9); 3.8396 (7.9); 3.6217 (0.8); 3.6177 (4.3); 3.6155
(2.4); 3.6115 (2.2); 3.6093 (1.7); 3.6074 (3.0); 3.6029 (3.2); 3.6011 (9.9); 3.5989 (3.4); 3.5950 (3.1); 3.5931 (1.7); 3.5908 (2.1);
3.5867 (2.2); 3.5845 (4.4); 3.5806 (0.7); 3.3225 (12.0); 3.2025 (1.2); 2.6706 (0.6); 2.5244 (1.9); 2.5197 (2.6); 2.5110 (34.2);
2.5064 (74.6); 2.5018 (104.1); 2.4972 (71.1); 2.4926 (30.7); 2.3289 (0.6); 2.1831 (0.8); 1.9950 (1.0); 1.9090 (0.8); 1.7808 (0.7);
1.7761 (4.3); 1.7744 (1.9); 1.7684 (3.4); 1.7641 (2.1); 1.7595 (12.5); 1.7548 (2.2); 1.7505 (3.4); 1.7429 (4.3); 1.7383 (0.6); 1.3555
(7.5); 1.2390 (0.8); 1.0897 (0.5); 1.0744 (0.5); 0.0079 (1.6); 0.0038 (0.7); −0.0002 (63.1); −0.0027 (2.6); −0.0043 (0.9); −0.0052
(0.6); −0.0060 (0.5); −0.0085 (1.8)
I-119: ¹H-NMR(400.6 MHz, d₆-DMSO):
δ = 8.2715 (1.8); 8.2653 (1.8); 8.1037 (1.1); 8.1017 (1.2); 8.0991 (1.4); 8.0971 (1.2); 8.0916 (1.2); 8.0896 (1.3); 8.0870 (1.3);
8.0849 (1.2); 8.0400 (0.8); 8.0338 (0.8); 8.0203 (1.1); 8.0189 (1.1); 8.0141 (1.0); 8.0127 (1.1); 7.9993 (0.9); 7.9930 (0.8); 7.9721
(1.0); 7.9675 (1.0); 7.9536 (1.2); 7.9515 (1.3); 7.9489 (1.2); 7.9468 (1.2); 7.9330 (1.3); 7.9283 (1.2); 7.6904 (1.3); 7.6883 (2.3);
7.6862 (1.3); 7.6698 (1.2); 7.6677 (2.0); 7.6655 (1.1); 7.3036 (1.2); 7.2980 (1.2); 7.2840 (2.2); 7.2819 (2.3); 7.2767 (1.2); 7.2753
(1.2); 7.2721 (1.2); 7.2696 (1.2); 7.2657 (1.2); 7.2633 (1.1); 7.2536 (1.1); 7.2511 (1.1); 5.7566 (16.0); 4.8364 (3.2); 4.4534 (0.6);
4.4280 (0.6); 4.0379 (1.4); 4.0201 (1.4); 3.3455 (0.5); 2.6703 (0.5); 2.5241 (1.3); 2.5194 (1.7); 2.5107 (26.6); 2.5061 (58.5);
2.5015 (82.1); 2.4969 (56.8); 2.4924 (25.0); 2.3286 (0.5); 1.9886 (6.7); 1.2355 (1.4); 1.1923 (2.0); 1.1745 (3.9); 1.1568 (1.8);
0.0080 (1.3); −0.0002 (50.4); −0.0051 (0.8); −0.0059 (0.6); −0.0067 (0.6); −0.0085 (1.5)
I-120: ¹H-NMR(400.6 MHz, CDCl3):
δ = 9.2075 (1.2); 8.2087 (1.6); 8.2067 (1.3); 8.2045 (1.3); 8.2024 (1.7); 8.0973 (1.0); 8.0953 (1.1); 8.0927 (1.2); 8.0906 (1.0);
8.0852 (1.1); 8.0832 (1.1); 8.0805 (1.1); 8.0785 (1.0); 7.8592 (0.9); 7.8530 (0.9); 7.8404 (1.1); 7.8381 (1.1); 7.8342 (1.0); 7.8318
(1.0); 7.8193 (1.0); 7.8160 (1.0); 7.8130 (1.1); 7.8114 (1.0); 7.7975 (1.0); 7.7954 (1.3); 7.7928 (1.0); 7.7907 (1.2); 7.7769 (1.2);
7.7722 (1.2); 7.6945 (1.3); 7.6923 (2.3); 7.6900 (1.3); 7.6739 (1.0); 7.6717 (1.6); 7.6694 (0.9); 7.2617 (18.1); 7.1651 (1.2); 7.1625
(1.2); 7.1529 (1.1); 7.1504 (1.2); 7.1467 (1.2); 7.1441 (1.1); 7.1345 (1.1); 7.1319 (1.0); 7.0147 (1.2); 7.0132 (1.2); 7.0073 (1.2);
7.0058 (1.1); 6.9936 (1.1); 6.9920 (1.1); 6.9862 (1.2); 6.9847 (1.1); 5.3005 (6.0); 4.9417 (11.9); 4.2445 (1.4); 4.2267 (4.5); 4.2088
(4.6); 4.1910 (1.6); 4.1802 (7.9); 4.1309 (0.6); 4.1131 (0.6); 3.0727 (16.0); 2.0455 (2.8); 1.5653 (1.8); 1.3064 (0.6); 1.3005 (5.8);
1.2827 (12.1); 1.2774 (1.2); 1.2648 (5.8); 1.2595 (2.1); 1.2417 (0.8); 0.0080 (0.8); −0.0002 (29.2); −0.0085 (0.8)
I-122: ¹H-NMR(400.0 MHz, d₆-DMSO):
δ = 8.3153 (3.4); 8.3024 (3.5); 8.1155 (1.8); 8.1128 (1.9); 8.1110 (1.8); 8.1033 (1.9); 8.1006 (1.9); 8.0989 (1.8); 8.0130 (1.3);
8.0084 (1.2); 7.9926 (1.9); 7.9881 (1.8); 7.9738 (1.5); 7.9692 (1.4); 7.6986 (3.3); 7.6780 (3.0); 7.3172 (3.0); 7.3149 (3.1); 7.3051
(3.2); 7.3025 (3.1); 7.2985 (1.8); 7.2962 (1.7); 7.2862 (1.5); 7.2840 (1.6); 7.2533 (3.8); 4.9274 (12.7); 3.3292 (16.0); 2.5245 (0.7);
2.5199 (1.0); 2.5110 (17.2); 2.5066 (37.9); 2.5021 (53.1); 2.4975 (38.0); 2.4930 (17.4); 2.0742 (2.6); 1.2344 (0.7)
I-123: ¹H-NMR(400.0 MHz, CDCl3):
δ = 8.6124 (0.7); 8.6062 (0.6); 8.5847 (3.8); 8.5788 (3.7); 8.3972 (3.8); 8.3926 (3.7); 8.3737 (0.6); 8.3690 (0.6); 8.1031 (1.4);
8.0988 (1.6); 8.0910 (1.4); 8.0877 (2.1); 8.0843 (1.2); 7.8538 (0.6); 7.8333 (0.8); 7.8289 (0.6); 7.8127 (2.2); 7.8083 (2.3); 7.8006
(2.7); 7.7975 (4.1); 7.7952 (3.7); 7.7930 (4.4); 7.7776 (0.9); 7.7719 (0.7); 7.7666 (0.5); 7.7490 (2.7); 7.7438 (3.8); 7.7385 (2.4);
7.2616 (45.4); 7.1818 (1.4); 7.1762 (1.3); 7.1696 (1.4); 7.1663 (1.4); 7.1636 (1.4); 7.1607 (1.4); 7.1547 (1.2); 7.1485 (1.2); 4.2681
(2.0); 4.2502 (6.2); 4.2324 (6.3); 4.2145 (2.1); 3.9438 (16.0); 1.6012 (0.9); 1.5160 (1.3); 1.4928 (0.8); 1.3029 (7.0); 1.2851 (14.1);
1.2672 (6.9); 0.0080 (1.0); −0.0002 (28.8); −0.0085 (1.3)
I-124: ¹H-NMR(400.6 MHz, CDCl3):
δ = 8.5780 (5.7); 8.5722 (5.7); 8.3895 (5.4); 8.3849 (5.6); 8.1785 (1.9); 8.1764 (2.1); 8.1739 (2.3); 8.1718 (2.1); 8.1664 (2.1);
8.1642 (2.2); 8.1618 (2.3); 8.1596 (2.1); 7.8614 (1.7); 7.8567 (1.7); 7.8433 (1.7); 7.8409 (3.2); 7.8388 (1.9); 7.8363 (3.2); 7.8228
(3.5); 7.8182 (3.6); 7.8068 (16.0); 7.7943 (3.4); 7.7917 (5.1); 7.7891 (3.7); 7.7738 (1.7); 7.7713 (2.3); 7.7686 (1.6); 7.7619 (5.0);
7.7571 (5.6); 7.7562 (5.9); 7.7514 (5.0); 7.2634 (11.6); 7.2335 (0.5); 7.2307 (3.1); 7.2277 (3.0); 7.2186 (2.6); 7.2155 (2.7); 7.2126
(2.8); 7.2095 (2.7); 7.2005 (2.4); 7.1974 (2.3); 5.2990 (10.0); 4.8158 (0.6); 4.5075 (0.6); 4.4897 (0.6); 4.4572 (0.6); 4.4393 (0.6);
4.2500 (0.5); 4.2457 (0.6); 4.2319 (0.5); 4.2278 (1.7); 4.2159 (0.5); 4.2100 (1.8); 4.1981 (0.5); 4.1921 (0.6); 3.9441 (0.9); 3.6124
(0.6); 3.6040 (0.6); 3.5829 (3.5); 1.6631 (1.2); 1.5532 (4.0); 1.5514 (4.0); 1.5261 (0.7); 1.4527 (0.8); 1.4511 (1.0); 1.4349 (1.4);
1.4170 (0.7); 1.4059 (0.7); 1.3881 (1.6); 1.3702 (1.0); 1.3423 (0.6); 1.3245 (1.2); 1.3195 (1.6); 1.3174 (0.8); 1.3148 (0.8); 1.3066
(0.8); 1.3017 (3.5); 1.2996 (1.7); 1.2970 (1.4); 1.2872 (0.9); 1.2840 (2.2); 1.2818 (1.0); 1.2792 (0.8); 1.2694 (0.6); 1.2668 (0.8);
1.2540 (1.4); −0.0002 (16.3)
I-125: ¹H-NMR(400.6 MHz, d₆-DMSO):
δ = 8.6638 (3.2); 8.6567 (3.2); 8.4120 (1.6); 8.4085 (2.9); 8.4078 (3.1); 8.4043 (1.6); 8.1420 (1.2); 8.1400 (1.3); 8.1374 (1.6);
8.1353 (1.4); 8.1299 (1.3); 8.1278 (1.6); 8.1253 (1.5); 8.1232 (1.4); 8.0530 (1.2); 8.0484 (1.2); 8.0344 (1.4); 8.0325 (1.5); 8.0298
(1.3); 8.0278 (1.5); 8.0139 (1.6); 8.0092 (1.4); 7.8793 (1.2); 7.8750 (1.3); 7.8723 (1.3); 7.8680 (1.2); 7.8552 (1.3); 7.8509 (1.4);
7.8482 (1.2); 7.8440 (1.2); 7.7843 (1.5); 7.7820 (2.8); 7.7797 (1.6); 7.7637 (1.3); 7.7615 (2.5); 7.7591 (1.4); 7.3589 (1.4); 7.3564
(1.5); 7.3468 (1.3); 7.3443 (1.4); 7.3403 (1.4); 7.3378 (1.3); 7.3282 (1.4); 7.3257 (1.4); 4.1727 (1.8); 4.1550 (6.2); 4.1372 (6.3);
4.1195 (1.9); 4.0828 (11.0); 3.3234 (122.3); 2.5244 (1.1); 2.5197 (1.5); 2.5109 (20.7); 2.5063 (46.0); 2.5017 (65.0); 2.4972 (45.4);
2.4926 (20.5); 1.5915 (1.0); 1.2011 (7.2); 1.1834 (16.0); 1.1657 (7.1); 0.0080 (1.2); 0.0030 (0.8); 0.0023 (1.5); −0.0002
(44.9); −0.0027 (2.4); −0.0034 (1.7); −0.0043 (1.1); −0.0051 (0.9); −0.0059 (0.8); −0.0067 (0.6); −0.0085 (1.5)
I-126: ¹H-NMR(400.6 MHz, CDCl3):
δ = 8.6098 (3.6); 8.6040 (3.7); 8.4036 (3.6); 8.3990 (3.7); 8.1282 (1.4); 8.1255 (1.5); 8.1180 (1.4); 8.1161 (1.5); 8.1134 (1.5);
7.8357 (1.0); 7.8310 (1.0); 7.8170 (1.4); 7.8151 (1.7); 7.8125 (1.4); 7.8104 (1.6); 7.7967 (1.7); 7.7920 (1.7); 7.7548 (2.9); 7.7498
(4.0); 7.7443 (2.8); 7.7239 (3.1); 7.7219 (1.9); 7.7034 (2.1); 7.2613 (65.4); 7.2064 (1.3); 7.2039 (1.3); 7.1943 (1.3); 7.1917 (1.4);
7.1880 (1.4); 7.1855 (1.3); 7.1758 (1.3); 7.1733 (1.2); 5.3003 (1.6); 3.9602 (16.0); 2.1038 (12.7); 1.2595 (0.6); 1.2556 (0.6);
0.0079 (1.2); −0.0002 (48.8); −0.0085 (1.6)
I-19: ¹H-NMR(400.6 MHz, CDCl3):
δ = 8.2083 (1.0); 8.2062 (0.8); 8.2043 (0.8); 8.2022 (1.1); 8.2002 (0.8); 8.0601 (0.7); 8.0580 (0.7); 8.0553 (0.8); 8.0533 (0.7);
8.0479 (0.7); 8.0458 (0.8); 8.0432 (0.7); 8.0411 (0.7); 7.8628 (0.6); 7.8567 (0.6); 7.8439 (0.7); 7.8417 (0.8); 7.8378 (0.7); 7.8355
(0.7); 7.8228 (0.7); 7.8166 (0.6); 7.7692 (0.6); 7.7645 (0.6); 7.7508 (0.7); 7.7486 (0.9); 7.7461 (0.7); 7.7438 (0.8); 7.7302 (0.8);
7.7254 (0.8); 7.6275 (0.9); 7.6252 (1.6); 7.6228 (1.0); 7.6068 (0.7); 7.6045 (1.2); 7.6022 (0.7); 7.2606 (22.4); 7.1083 (0.8); 7.1058
(0.8); 7.0962 (0.8); 7.0936 (0.8); 7.0900 (0.8); 7.0874 (0.7); 7.0778 (0.8); 7.0752 (0.7); 6.9955 (0.8); 6.9939 (0.8); 6.9881 (0.8);
6.9865 (0.8); 6.9743 (0.8); 6.9727 (0.8); 6.9669 (0.8); 6.9654 (0.8); 5.2765 (1.5); 5.2591 (1.5); 3.8013 (16.0); 3.7263 (0.8); 3.7238
(0.7); 1.7211 (6.1); 1.7037 (6.0); 1.5462 (6.7); 1.2648 (0.6); 1.2596 (0.6); 0.8820 (1.4); 0.8643 (0.5); 0.0080 (0.9); −0.0002
(30.8); −0.0085 (0.8)
I-20: ¹H-NMR(400.0 MHz, d₆-DMSO):
δ = 8.6435 (2.7); 8.6360 (0.8); 8.4120 (0.7); 8.3999 (0.8); 8.2493 (3.0); 8.2431 (3.3); 8.0967 (2.5); 8.0844 (2.4); 8.0166 (1.1);
8.0105 (1.2); 7.9964 (1.8); 7.9899 (2.0); 7.9743 (2.5); 7.9695 (2.9); 7.9537 (3.0); 7.9349 (2.2); 7.9302 (2.1); 7.6619 (4.0); 7.6411
(3.8); 7.6169 (0.9); 7.4066 (0.5); 7.3000 (2.5); 7.2935 (2.8); 7.2778 (4.1); 7.2634 (2.2); 7.2591 (2.0); 7.2448 (1.9); 6.3103 (0.5);
5.7568 (16.0); 5.1154 (0.7); 5.0981 (2.5); 5.0807 (2.8); 5.0641 (1.5); 5.0461 (0.8); 4.0379 (1.0); 4.0202 (1.0); 3.3894 (3.8); 3.3388
(6.7); 2.6714 (1.0); 2.5415 (3.2); 2.5247 (2.1); 2.5112 (51.6); 2.5067 (112.5); 2.5021 (157.4); 2.4975 (111.9); 2.4931 (50.8);
2.3332 (0.7); 2.3289 (0.9); 2.1174 (0.6); 1.9888 (4.3); 1.9063 (1.1); 1.5668 (9.4); 1.5494 (9.8); 1.5257 (3.2); 1.3380 (1.5); 1.2977
(0.8); 1.2816 (0.6); 1.2585 (0.9); 1.2348 (5.4); 1.1924 (1.2); 1.1746 (2.3); 1.1567 (1.2); 1.1403 (1.4); 0.8722 (0.6); 0.8537 (1.0);
0.0080 (2.8); −0.0002 (95.8); −0.0085 (3.0)
I-21: ¹H-NMR(400.6 MHz, d₆-DMSO):
δ = 8.6673 (2.8); 8.4204 (0.7); 8.4107 (0.7); 8.4084 (0.7); 8.3131 (5.6); 8.3002 (5.8); 8.1181 (2.9); 8.1161 (3.2); 8.1135 (3.6);
8.1115 (3.2); 8.1060 (3.2); 8.1040 (3.5); 8.1014 (3.5); 8.0994 (3.1); 8.0111 (2.5); 8.0064 (2.3); 7.9925 (3.5); 7.9905 (3.7); 7.9880
(3.5); 7.9859 (3.2); 7.9720 (3.2); 7.9673 (3.2); 7.9496 (0.7); 7.9451 (0.6); 7.6732 (6.0); 7.6548 (3.8); 7.6527 (5.4); 7.6388 (1.4);
7.6181 (1.1); 7.4282 (0.6); 7.4228 (0.7); 7.4119 (0.7); 7.4063 (1.0); 7.3997 (0.8); 7.3889 (0.7); 7.3834 (0.8); 7.3548 (0.8); 7.3479
(0.7); 7.3385 (0.9); 7.3136 (6.3); 7.3012 (6.5); 7.2993 (6.6); 7.2954 (5.3); 7.2830 (3.2); 7.2806 (2.9); 7.2438 (5.5); 6.3099 (1.0);
6.2869 (1.0); 6.1606 (0.5); 6.1577 (0.5); 6.1444 (1.0); 6.1416 (0.9); 5.7569 (12.6); 5.1530 (1.1); 5.1358 (3.8); 5.1184 (4.0); 5.0995
(1.6); 5.0816 (0.9); 4.0557 (0.8); 4.0378 (2.5); 4.0200 (2.5); 4.0022 (0.9); 3.3889 (2.1); 3.3268 (7.8); 2.6754 (0.9); 2.6708 (1.3);
2.6661 (0.9); 2.5412 (1.4); 2.5346 (1.6); 2.5301 (2.4); 2.5246 (4.6); 2.5197 (6.4); 2.5110 (71.1); 2.5065 (148.2); 2.5019 (202.3);
2.4973 (146.2); 2.4928 (72.4); 2.4720 (5.0); 2.4673 (4.4); 2.3335 (1.3); 2.3290 (1.6); 2.3245 (1.3); 2.1172 (1.5); 1.9888 (11.1);
1.9079 (2.2); 1.7542 (0.5); 1.5867 (15.4); 1.5693 (16.0); 1.5641 (6.1); 1.5465 (3.8); 1.5201 (0.7); 1.4509 (0.6); 1.2983 (1.3);
1.2585 (2.1); 1.2355 (6.4); 1.1923 (3.6); 1.1745 (6.7); 1.1567 (3.4); 1.1404 (4.1); 1.1067 (1.6); 1.0573 (1.2); 1.0419 (1.2); 1.0223
(0.8); 1.0133 (0.7); 1.0066 (0.9); 0.9997 (0.6); 0.8884 (0.7); 0.8726 (1.3); 0.8537 (1.5); 0.8363 (0.7); 0.1457 (0.5); 0.0279 (1.3);
0.0080 (5.6); −0.0002 (170.9); −0.0085 (8.4); −0.0344 (1.1); −0.0377 (1.0); −0.1494 (0.7)
I-22: ¹H-NMR(400.0 MHz, d₆-DMSO):
δ = 9.2496 (7.1); 8.8883 (13.9); 8.6788 (2.0); 8.0732 (1.5); 8.0610 (1.5); 8.0130 (1.1); 8.0084 (1.0); 7.9923 (1.8); 7.9738 (1.5);
7.9691 (1.2); 7.7084 (2.9); 7.6877 (2.6); 7.6393 (1.0); 7.6184 (0.9); 7.3226 (0.7); 7.3055 (0.7); 7.2939 (1.8); 7.2793 (1.5); 7.2753
(1.4); 7.2608 (1.4); 5.7568 (5.4); 5.1972 (0.7); 5.1797 (2.3); 5.1622 (2.2); 5.1451 (0.6); 5.1159 (0.6); 5.0986 (0.8); 3.3203 (16.0);
2.6702 (1.0); 2.5239 (2.8); 2.5192 (4.0); 2.5104 (53.4); 2.5059 (115.4); 2.5013 (161.0); 2.4968 (114.4); 2.4923 (52.1); 2.3328
(0.9); 2.3284 (1.2); 2.1168 (0.7); 1.6108 (7.8); 1.5935 (7.6); 1.5741 (2.4); 1.5566 (2.4); 1.5279 (0.6); 1.4686 (0.8); 1.4513 (1.5);
1.2983 (1.6); 1.2583 (2.4); 1.2346 (7.0); 1.1404 (1.9); 1.1068 (1.1); 1.0571 (0.9); 1.0418 (0.9); 1.0135 (2.6); 0.9998 (0.8); 0.8888
(0.5); 0.8720 (1.0); 0.8615 (0.9); 0.8535 (1.4); 0.8363 (0.7); 0.1460 (0.6); 0.0080 (4.0); −0.0002 (142.7); −0.0085 (4.7); −0.1496 (0.6)
I-23: ¹H-NMR(400.6 MHz, CDCl3):
δ = 8.3881 (2.3); 8.3841 (2.4); 8.3764 (2.4); 8.3724 (2.4); 8.1389 (2.2); 8.1327 (2.4); 7.8626 (1.1); 7.8564 (1.0); 7.8438 (1.2);
7.8414 (1.3); 7.8376 (1.2); 7.8352 (1.3); 7.8227 (1.2); 7.8164 (1.1); 7.8012 (2.4); 7.7972 (2.4); 7.7811 (2.6); 7.7771 (2.6); 7.3335
(2.5); 7.3219 (2.5); 7.3135 (2.4); 7.3018 (2.3); 7.2617 (11.3); 6.9351 (1.5); 6.9280 (1.6); 6.9138 (1.5); 6.9067 (1.5); 4.9187 (16.0);
4.2820 (1.9); 4.2642 (6.0); 4.2464 (6.1); 4.2286 (2.0); 2.0453 (1.3); 1.5616 (5.4); 1.3002 (7.2); 1.2824 (14.6); 1.2646 (7.6); 1.2599
(1.5); 0.8821 (1.3); 0.8644 (0.5); −0.0002 (16.8); −0.0085 (0.6)
I-24: ¹H-NMR(400.6 MHz, CDCl3):
δ = 8.3500 (1.3); 8.3460 (1.4); 8.3384 (1.4); 8.3343 (1.4); 8.1353 (1.0); 8.1332 (0.8); 8.1311 (0.9); 8.1291 (1.1); 7.8576 (0.6);
7.8513 (0.6); 7.8388 (0.7); 7.8364 (0.7); 7.8325 (0.7); 7.8301 (0.7); 7.8176 (0.6); 7.8114 (0.6); 7.8028 (1.4); 7.7988 (1.4); 7.7827
(1.5); 7.7787 (1.5); 7.3170 (1.5); 7.3053 (1.5); 7.2969 (1.4); 7.2852 (1.4); 7.2619 (7.1); 6.9300 (0.7); 6.9286 (0.8); 6.9226 (0.8);
6.9211 (0.8); 6.9088 (0.8); 6.9073 (0.8); 6.9013 (0.8); 6.8998 (0.7); 5.2593 (1.5); 5.2419 (1.5); 4.1309 (0.6); 4.1130 (0.6); 3.7563
(16.0); 2.0453 (2.8); 1.6961 (6.3); 1.6787 (6.3); 1.5609 (3.4); 1.2773 (1.0); 1.2645 (0.8); 1.2595 (2.1); 1.2416 (0.9); 0.8818 (1.5);
0.8641 (0.6); −0.0002 (11.2)
I-25: ¹H-NMR(400.6 MHz, CDCl3):
δ = 8.3707 (3.6); 8.3667 (3.8); 8.3590 (3.8); 8.3550 (3.8); 8.1350 (3.4); 8.1287 (3.5); 7.8620 (1.7); 7.8557 (1.6); 7.8433 (2.0);
7.8408 (2.1); 7.8371 (2.0); 7.8346 (1.9); 7.8221 (1.8); 7.8158 (1.8); 7.8111 (3.8); 7.8071 (3.8); 7.7910 (4.1); 7.7870 (4.0); 7.3331
(4.0); 7.3214 (4.0); 7.3130 (3.8); 7.3012 (3.6); 7.2606 (50.9); 6.9398 (2.3); 6.9325 (2.4); 6.9186 (2.2); 6.9112 (2.2); 5.3337 (1.2);
5.3162 (4.6); 5.2988 (4.7); 5.2814 (1.3); 1.7459 (16.0); 1.7284 (15.9); 1.2644 (2.1); 0.8990 (1.1); 0.8821 (3.8); 0.8644 (1.5);
0.0080 (2.4); −0.0002 (81.0); −0.0086 (2.8)
I-26: ¹H-NMR(400.6 MHz, CDCl3):
δ = 8.3888 (3.3); 8.3872 (3.5); 8.3849 (3.6); 8.3771 (3.5); 8.3756 (3.7); 8.3732 (3.6); 8.1350 (4.4); 8.1291 (4.6); 7.8596 (1.8);
7.8534 (1.8); 7.8387 (2.6); 7.8327 (2.5); 7.8197 (2.0); 7.8135 (2.0); 7.8030 (4.1); 7.7990 (4.3); 7.7830 (4.6); 7.7790 (4.6); 7.3362
(4.0); 7.3245 (4.0); 7.3161 (3.9); 7.3044 (3.9); 7.2668 (73.3); 6.9349 (3.2); 6.9275 (3.3); 6.9137 (3.2); 6.9063 (3.2); 5.3020 (1.2);
4.9259 (16.0); 4.9220 (14.5); 4.0916 (0.8); 4.0876 (0.8); 2.5953 (1.2); 2.0982 (1.2); 1.2637 (2.1); 0.8983 (1.0); 0.8819 (2.9);
0.8641 (1.3); 0.0079 (2.0); −0.0002 (84.1); −0.0084 (4.0); −0.0266 (0.8)
I-27: ¹H-NMR(400.6 MHz, CDCl3):
δ = 8.3542 (2.5); 8.3502 (2.7); 8.3425 (2.7); 8.3385 (2.8); 8.1323 (2.4); 8.1261 (2.6); 7.8582 (1.2); 7.8520 (1.2); 7.8394 (1.4);
7.8371 (1.5); 7.8332 (1.4); 7.8309 (1.5); 7.8183 (1.3); 7.8120 (1.3); 7.7986 (2.6); 7.7946 (2.7); 7.7786 (2.9); 7.7745 (2.9); 7.3154
(2.8); 7.3038 (2.8); 7.2954 (2.7); 7.2837 (2.6); 7.2613 (17.2); 6.9288 (1.8); 6.9215 (1.8); 6.9076 (1.7); 6.9003 (1.7); 5.3002 (1.7);
5.2577 (0.9); 5.2403 (3.3); 5.2230 (3.4); 5.2056 (1.0); 4.2408 (1.8); 4.2230 (5.9); 4.2052 (6.2); 4.1875 (2.1); 2.1730 (1.1); 1.6909
(13.0); 1.6735 (13.0); 1.2677 (7.8); 1.2499 (16.0); 1.2321 (7.8); 0.0079 (0.8); −0.0002 (26.7); −0.0084 (1.2)
I-28: ¹H-NMR(400.6 MHz, CDCl3):
δ = 8.3882 (1.4); 8.3842 (1.5); 8.3765 (1.5); 8.3725 (1.5); 8.1409 (1.3); 8.1390 (1.0); 8.1367 (1.0); 8.1346 (1.3); 7.8628 (0.6);
7.8565 (0.6); 7.8441 (0.7); 7.8416 (0.8); 7.8378 (0.7); 7.8353 (0.8); 7.8229 (0.7); 7.8166 (0.7); 7.8043 (1.5); 7.8003 (1.5); 7.7842
(1.7); 7.7802 (1.6); 7.3365 (1.6); 7.3248 (1.6); 7.3164 (1.5); 7.3047 (1.5); 7.2618 (9.0); 6.9370 (0.8); 6.9358 (0.9); 6.9297 (0.8);
6.9283 (0.9); 6.9159 (0.8); 6.9145 (0.8); 6.9084 (0.8); 6.9070 (0.8); 4.9368 (8.7); 3.7924 (16.0); 1.5637 (6.8); −0.0002 (12.8)
I-29: ¹H-NMR(400.6 MHz, CDCl3):
δ = 8.2558 (0.6); 8.2541 (0.7); 8.2522 (0.7); 8.2505 (0.6); 8.2442 (0.6); 8.2424 (0.7); 8.2406 (0.7); 8.2389 (0.7); 8.1338 (0.9);
8.1318 (0.7); 8.1296 (0.8); 8.1276 (1.0); 7.8457 (0.6); 7.8432 (0.6); 7.8395 (0.6); 7.8370 (0.6); 7.8245 (0.6); 7.8183 (0.5); 7.5051
(0.5); 7.4881 (0.7); 7.4845 (0.8); 7.4820 (0.6); 7.4650 (0.7); 7.4613 (0.7); 7.3557 (0.6); 7.3467 (0.7); 7.3441 (0.7); 7.3351 (1.1);
7.3260 (0.5); 7.3234 (0.6); 7.2612 (21.3); 6.9646 (0.6); 6.9631 (0.7); 6.9571 (0.7); 6.9556 (0.7); 6.9434 (0.6); 6.9418 (0.7); 6.9359
(0.6); 6.9344 (0.6); 4.9905 (6.9); 4.3640 (1.6); 4.3556 (0.9); 4.3523 (1.7); 4.3486 (0.9); 4.3404 (1.8); 3.6273 (2.0); 3.6190 (0.9);
3.6154 (1.8); 3.6121 (1.0); 3.6037 (1.9); 3.3659 (16.0); 1.5532 (10.3); 0.0079 (0.6); −0.0002 (24.7); −0.0085 (0.9)
I-30: ¹H-NMR(400.6 MHz, CDCl3):
δ = 8.2583 (1.2); 8.2564 (1.3); 8.2466 (1.3); 8.2448 (1.3); 8.1494 (1.7); 8.1431 (1.8); 7.8692 (0.9); 7.8629 (0.9); 7.8505 (1.0);
7.8480 (1.1); 7.8443 (1.0); 7.8418 (1.1); 7.8293 (1.0); 7.8231 (1.0); 7.5260 (0.9); 7.5223 (0.9); 7.5190 (0.6); 7.5053 (1.2); 7.5019
(1.5); 7.4993 (1.0); 7.4823 (1.2); 7.4785 (1.2); 7.3724 (1.2); 7.3633 (1.3); 7.3608 (1.2); 7.3517 (2.0); 7.3426 (0.9); 7.3401 (0.9);
7.3310 (0.9); 7.2606 (84.6); 6.9705 (1.2); 6.9644 (1.2); 6.9492 (1.2); 6.9431 (1.2); 5.0371 (13.7); 4.8404 (16.0); 1.5427 (8.1);
0.0081 (2.8); −0.0002 (97.2); −0.0085 (3.3)
I-31: ¹H-NMR(400.6 MHz, CDCl3):
δ = 8.2951 (1.6); 8.2934 (1.9); 8.2915 (1.9); 8.2898 (1.8); 8.2835 (1.8); 8.2818 (2.0); 8.2799 (2.0); 8.2782 (1.8); 8.1602 (2.5);
8.1582 (1.9); 8.1559 (2.0); 8.1539 (2.7); 7.8498 (1.4); 7.8435 (1.4); 7.8312 (1.5); 7.8286 (1.7); 7.8249 (1.6); 7.8224 (1.6); 7.8100
(1.5); 7.8037 (1.5); 7.5386 (1.3); 7.5349 (1.4); 7.5179 (1.9); 7.5156 (1.9); 7.5143 (2.1); 7.5121 (1.6); 7.4950 (1.8); 7.4913 (1.8);
7.3980 (1.8); 7.3888 (2.0); 7.3864 (1.9); 7.3773 (3.2); 7.3681 (1.4); 7.3657 (1.5); 7.3566 (1.3); 7.2627 (20.5); 6.9737 (1.8); 6.9723
(1.8); 6.9663 (1.9); 6.9648 (1.8); 6.9526 (1.7); 6.9510 (1.8); 6.9451 (1.8); 6.9436 (1.8); 6.6525 (0.6); 5.9488 (0.6); 5.9350 (1.3);
5.9230 (0.8); 5.9214 (0.7); 5.9093 (1.4); 5.9060 (0.8); 5.8956 (0.7); 5.8922 (1.6); 5.8801 (0.9); 5.8786 (0.9); 5.8665 (1.6); 5.8528
(0.8); 5.2850 (0.8); 5.2808 (2.0); 5.2776 (2.2); 5.2734 (1.0); 5.2422 (0.7); 5.2379 (1.8); 5.2347 (1.9); 5.2305 (0.9); 5.1997 (0.9);
5.1961 (2.3); 5.1928 (2.3); 5.1893 (0.9); 5.1740 (0.8); 5.1704 (2.2); 5.1671 (2.2); 5.1636 (0.9); 4.8925 (16.0); 4.0378 (1.1); 4.0338
(2.1); 4.0299 (1.3); 4.0233 (2.2); 4.0196 (3.8); 4.0158 (2.3); 4.0093 (1.4); 4.0053 (2.2); 4.0013 (1.3); 2.0454 (0.7); 1.5810 (5.4);
1.2595 (0.7); 1.2558 (0.5); −0.0002 (19.4); −0.0085 (0.7)
I-32: ¹H-NMR(400.6 MHz, CDCl3):
δ = 8.2221 (1.4); 8.2204 (1.6); 8.2185 (1.6); 8.2169 (1.5); 8.2105 (1.5); 8.2087 (1.7); 8.2069 (1.6); 8.2053 (1.4); 8.1253 (2.2);
8.1233 (1.7); 8.1191 (2.2); 7.8566 (1.2); 7.8504 (1.1); 7.8379 (1.3); 7.8354 (1.4); 7.8317 (1.3); 7.8292 (1.3); 7.8167 (1.2); 7.8105
(1.2); 7.5154 (1.2); 7.5117 (1.2); 7.4947 (1.6); 7.4914 (2.0); 7.4884 (1.3); 7.4715 (1.5); 7.4678 (1.4); 7.3408 (1.4); 7.3318 (1.6);
7.3291 (1.5); 7.3201 (2.5); 7.3111 (1.2); 7.3085 (1.2); 7.2996 (1.1); 7.2610 (31.5); 6.9621 (1.5); 6.9607 (1.5); 6.9546 (1.6); 6.9532
(1.5); 6.9409 (1.5); 6.9394 (1.5); 6.9334 (1.5); 6.9320 (1.4); 5.2849 (0.9); 5.2675 (3.2); 5.2501 (3.2); 5.2328 (0.9); 4.2553 (1.3);
4.2386 (4.2); 4.2377 (4.2); 4.2206 (4.4); 4.2027 (1.5); 1.6951 (12.4); 1.6777 (12.4); 1.5489 (11.6); 1.2730 (7.7); 1.2552 (16.0);
1.2374 (7.5); 0.0080 (1.3); −0.0002 (47.7); −0.0085 (1.4)
I-33: ¹H-NMR(400.0 MHz, CDCl3):
δ = 8.2157 (1.3); 8.2139 (1.3); 8.2040 (1.3); 8.2023 (1.3); 8.1334 (1.7); 8.1272 (1.8); 7.8631 (0.8); 7.8569 (0.8); 7.8443 (1.0);
7.8419 (1.0); 7.8381 (1.0); 7.8357 (1.0); 7.8231 (0.9); 7.8168 (0.8); 7.5072 (0.8); 7.5035 (0.8); 7.4865 (1.1); 7.4831 (1.4); 7.4803
(1.0); 7.4633 (1.0); 7.4596 (1.0); 7.3355 (1.0); 7.3265 (1.2); 7.3239 (1.1); 7.3149 (1.8); 7.3058 (0.9); 7.3032 (0.9); 7.2942 (0.8);
7.2615 (20.5); 6.9557 (1.2); 6.9495 (1.2); 6.9344 (1.1); 6.9283 (1.1); 5.2825 (0.6); 5.2651 (2.3); 5.2477 (2.3); 5.2303 (0.6); 4.2531
(1.0); 4.2369 (2.9); 4.2354 (3.0); 4.2190 (3.1); 4.2176 (3.0); 4.2011 (1.1); 2.0453 (0.6); 1.6936 (8.8); 1.6762 (8.7); 1.5569 (16.0);
1.2719 (5.6); 1.2594 (1.2); 1.2541 (11.3); 1.2363 (5.3); 0.8819 (1.1); 0.0079 (0.6); −0.0002 (20.9); −0.0085 (0.6)
I-34: ¹H-NMR(400.6 MHz, CDCl3):
δ = 8.2178 (0.5); 8.2160 (0.6); 8.2141 (0.7); 8.2061 (0.6); 8.2043 (0.7); 8.2025 (0.7); 8.2008 (0.7); 8.1303 (0.8); 8.1240 (0.9);
7.8394 (0.5); 7.8369 (0.6); 7.8332 (0.6); 7.8308 (0.6); 7.8120 (0.5); 7.5048 (0.5); 7.4878 (0.6); 7.4843 (0.8); 7.4816 (0.6); 7.4646
(0.6); 7.4609 (0.6); 7.3389 (0.6); 7.3299 (0.7); 7.3273 (0.6); 7.3183 (1.1); 7.3092 (0.5); 7.3067 (0.5); 7.2603 (61.6); 6.9554 (0.6);
6.9496 (0.6); 6.9480 (0.6); 6.9358 (0.6); 6.9343 (0.6); 6.9284 (0.6); 6.9268 (0.6); 5.3051 (1.3); 5.2877 (1.3); 4.3625 (0.5); 4.3511
(0.8); 4.3490 (0.7); 4.3374 (0.6); 4.3325 (0.6); 4.3214 (0.7); 4.3193 (0.8); 4.3083 (0.6); 3.7113 (0.8); 3.7077 (0.8); 3.7005 (0.9);
3.6963 (1.2); 3.6942 (1.1); 3.6866 (0.7); 3.6830 (0.8); 3.6120 (0.8); 3.6077 (0.8); 3.6013 (1.8); 3.5967 (1.0); 3.5939 (1.3); 3.5879
(1.3); 3.5857 (1.4); 3.5109 (2.0); 3.5054 (0.5); 3.5022 (0.9); 3.4982 (1.8); 3.4876 (1.0); 3.3605 (16.0); 3.3156 (0.6); 1.7081 (4.8);
1.6907 (4.8); 1.6255 (0.6); 0.0079 (2.5); −0.0002 (95.6); −0.0085 (2.9)
I-35: ¹H-NMR(400.6 MHz, CDCl3):
δ = 8.2149 (0.6); 8.2130 (0.7); 8.2112 (0.7); 8.2095 (0.7); 8.2032 (0.6); 8.2015 (0.7); 8.1996 (0.7); 8.1979 (0.7); 8.1293 (0.9);
8.1231 (1.0); 7.8593 (0.5); 7.8406 (0.6); 7.8381 (0.6); 7.8343 (0.6); 7.8319 (0.6); 7.8194 (0.5); 7.8131 (0.5); 7.5075 (0.5); 7.5038
(0.5); 7.4869 (0.7); 7.4834 (0.9); 7.4806 (0.6); 7.4636 (0.6); 7.4599 (0.6); 7.3355 (0.7); 7.3266 (0.7); 7.3239 (0.7); 7.3149 (1.2);
7.3059 (0.5); 7.3033 (0.5); 7.2607 (20.0); 6.9575 (0.6); 6.9560 (0.7); 6.9500 (0.7); 6.9486 (0.7); 6.9362 (0.6); 6.9347 (0.7); 6.9288
(0.6); 6.9273 (0.6); 5.3137 (1.4); 5.2963 (1.4); 4.3344 (0.8); 4.3281 (0.9); 4.3247 (0.9); 4.3213 (1.1); 4.3171 (1.1); 4.3138 (1.0);
4.3100 (1.0); 4.3045 (0.9); 3.6013 (1.0); 3.5973 (1.0); 3.5886 (1.7); 3.5860 (1.8); 3.5772 (0.9); 3.5733 (1.0); 3.3304 (16.0); 1.7120
(5.3); 1.6946 (5.2); 1.5480 (12.0); 0.0080 (0.8); −0.0002 (30.1); −0.0085 (0.9)
I-36: ¹H-NMR(599.6 MHz, CDCl3):
δ = 8.6302 (2.0); 8.2879 (2.2); 8.2811 (2.2); 8.1464 (3.1); 7.8591 (0.9); 7.8452 (1.7); 7.8326 (1.0); 7.5382 (1.0); 7.5230 (2.1);
7.5091 (1.2); 7.4030 (0.9); 7.3962 (1.5); 7.3894 (1.6); 7.3829 (1.2); 7.2602 (32.6); 6.9805 (1.7); 6.9665 (1.7); 4.9133 (10.7);
4.1279 (0.6); 4.1159 (0.6); 2.9962 (33.0); 2.0444 (2.6); 1.5479 (41.5); 1.2710 (1.1); 1.2592 (2.1); 1.2474 (0.9); 0.8933 (0.4);
0.8821 (0.9); 0.8702 (0.4); −0.0001 (50.0)
I-37: ¹H-NMR(400.0 MHz, CDCl3):
δ = 8.2569 (0.9); 8.2435 (1.0); 8.1350 (1.2); 8.1285 (1.2); 7.8644 (0.5); 7.8580 (0.5); 7.8432 (0.7); 7.8395 (0.7); 7.8244 (0.6);
7.8184 (0.6); 7.5196 (0.6); 7.5095 (0.5); 7.5059 (0.5); 7.4862 (1.0); 7.4657 (0.7); 7.4618 (0.7); 7.3581 (0.8); 7.3487 (0.9); 7.3373
(1.1); 7.3282 (0.6); 7.2606 (108.8); 6.9966 (0.6); 6.9641 (0.8); 6.9565 (0.9); 6.9427 (0.8); 6.9351 (0.8); 4.9798 (7.6); 4.3788 (1.7);
4.3668 (1.9); 4.3546 (1.9); 3.7371 (2.0); 3.7287 (1.2); 3.7250 (2.0); 3.7130 (1.8); 3.6403 (1.4); 3.6293 (1.9); 3.6241 (1.5); 3.6172
(2.6); 3.5369 (2.5); 3.5300 (1.4); 3.5249 (1.9); 3.5137 (1.4); 3.3724 (16.0); 1.5424 (12.2); 0.0080 (1.6); −0.0002 (66.1); −0.0085 (2.1)
I-38: ¹H-NMR(400.0 MHz, CDCl3):
δ = 8.2633 (1.7); 8.2517 (1.7); 8.1287 (2.3); 8.1225 (2.3); 7.8610 (1.1); 7.8546 (1.1); 7.8422 (1.3); 7.8397 (1.3); 7.8359 (1.3);
7.8335 (1.2); 7.8210 (1.2); 7.8147 (1.1); 7.5198 (0.5); 7.5167 (1.1); 7.5130 (1.1); 7.4959 (1.5); 7.4925 (1.9); 7.4900 (1.2); 7.4728
(1.4); 7.4691 (1.3); 7.3620 (1.3); 7.3529 (1.6); 7.3504 (1.4); 7.3413 (2.3); 7.3322 (1.1); 7.3297 (1.1); 7.3206 (1.0); 7.2610 (70.8);
6.9676 (1.5); 6.9614 (1.5); 6.9476 (1.5); 6.9401 (1.4); 4.9380 (16.0); 4.2936 (1.9); 4.2757 (6.1); 4.2578 (6.2); 4.2400 (2.0); 1.5488
(5.2); 1.3054 (7.3); 1.2875 (15.0); 1.2697 (7.2); 0.0079 (1.2); −0.0002 (42.2); −0.0085 (1.2)
I-39: ¹H-NMR(400.0 MHz, CDCl3):
δ = 8.2585 (1.8); 8.2567 (1.7); 8.2468 (1.8); 8.2450 (1.7); 8.1373 (2.3); 8.1311 (2.3); 7.8676 (1.1); 7.8613 (1.1); 7.8488 (1.3);
7.8463 (1.4); 7.8426 (1.3); 7.8401 (1.3); 7.8276 (1.2); 7.8214 (1.1); 7.5198 (0.5); 7.5087 (1.1); 7.5049 (1.1); 7.4879 (1.6); 7.4845
(1.8); 7.4649 (1.4); 7.4611 (1.4); 7.3565 (1.4); 7.3474 (1.7); 7.3449 (1.4); 7.3358 (2.4); 7.3268 (1.2); 7.3242 (1.2); 7.3151 (1.0);
7.2609 (90.8); 6.9629 (1.6); 6.9567 (1.6); 6.9430 (1.5); 6.9356 (1.6); 4.9367 (16.0); 4.2909 (2.0); 4.2730 (6.1); 4.2552 (6.2);
4.2373 (2.0); 1.5461 (10.5); 1.3038 (7.5); 1.2859 (15.3); 1.2680 (7.3); 0.0079 (1.6); −0.0002 (53.6); −0.0085 (1.6)
I-40: ¹H-NMR(400.6 MHz, CDCl3):
δ = 8.2292 (1.4); 8.2273 (1.6); 8.2257 (1.7); 8.2238 (1.6); 8.2176 (1.4); 8.2157 (1.7); 8.2140 (1.7); 8.2122 (1.5); 8.1361 (2.3);
8.1299 (2.4); 7.7748 (1.1); 7.7686 (1.1); 7.7557 (1.3); 7.7538 (1.4); 7.7495 (1.3); 7.7476 (1.4); 7.7347 (1.1); 7.7285 (1.1); 7.4624
(1.1); 7.4587 (1.2); 7.4418 (1.4); 7.4383 (2.1); 7.4353 (1.4); 7.4184 (1.4); 7.4147 (1.4); 7.2867 (1.4); 7.2778 (1.6); 7.2751 (1.6);
7.2661 (2.6); 7.2619 (16.9); 7.2573 (1.7); 7.2545 (1.4); 7.2456 (1.0); 6.9180 (1.6); 6.9119 (1.5); 6.9106 (1.6); 6.8969 (1.5); 6.8895
(1.5); 4.8875 (16.0); 4.2745 (2.0); 4.2567 (6.1); 4.2389 (6.2); 4.2210 (2.0); 1.6146 (0.6); 1.5998 (0.8); 1.5948 (1.0); 1.5875 (0.8);
1.5806 (2.4); 1.5729 (1.3); 1.5655 (1.6); 1.5610 (1.6); 1.5464 (0.7); 1.3010 (7.6); 1.2832 (15.3); 1.2653 (7.2); 0.7978 (1.5); 0.7927
(4.1); 0.7902 (3.5); 0.7842 (5.6); 0.7792 (5.1); 0.7752 (2.6); 0.7678 (2.4); 0.7637 (3.7); 0.7609 (1.9); 0.7582 (1.7); 0.0079
(0.8); −0.0002 (24.0); −0.0085 (0.8)
I-41: ¹H-NMR(400.6 MHz, CDCl3):
δ = 8.2220 (0.7); 8.2202 (0.8); 8.2183 (0.8); 8.2166 (0.7); 8.2103 (0.8); 8.2085 (0.8); 8.2067 (0.8); 8.2051 (0.7); 8.1279 (1.1);
8.1258 (0.8); 8.1236 (0.9); 8.1218 (1.1); 7.8570 (0.6); 7.8507 (0.6); 7.8382 (0.7); 7.8357 (0.7); 7.8320 (0.7); 7.8295 (0.7); 7.8170
(0.6); 7.8108 (0.6); 7.5206 (0.7); 7.5169 (0.6); 7.5000 (0.8); 7.4970 (1.0); 7.4936 (0.7); 7.4767 (0.8); 7.4730 (0.7); 7.3441 (0.7);
7.3351 (0.8); 7.3325 (0.8); 7.3234 (1.3); 7.3145 (0.6); 7.3118 (0.6); 7.3028 (0.5); 7.2611 (44.6); 6.9634 (0.8); 6.9619 (0.8); 6.9559
(0.8); 6.9544 (0.8); 6.9421 (0.8); 6.9406 (0.8); 6.9347 (0.8); 6.9331 (0.7); 5.2926 (1.5); 5.2752 (1.5); 3.7708 (16.0); 1.7001 (6.4);
1.6828 (6.4); 1.5547 (1.0); 0.0080 (0.8); −0.0002 (27.8); −0.0085 (0.8)
I-42: ¹H-NMR(400.6 MHz, CDCl3):
δ = 8.2315 (0.7); 8.2296 (0.9); 8.2279 (0.9); 8.2261 (0.8); 8.2198 (0.8); 8.2179 (0.9); 8.2162 (0.9); 8.2144 (0.8); 8.1366 (1.2);
8.1303 (1.2); 7.7747 (0.6); 7.7685 (0.6); 7.7557 (0.7); 7.7536 (0.8); 7.7495 (0.7); 7.7474 (0.8); 7.7346 (0.7); 7.7284 (0.7); 7.5191
(0.6); 7.4641 (0.6); 7.4604 (0.7); 7.4436 (0.8); 7.4401 (1.2); 7.4370 (0.7); 7.4201 (0.8); 7.4164 (0.8); 7.2895 (0.7); 7.2805 (0.9);
7.2778 (0.9); 7.2689 (1.8); 7.2607 (110.2); 7.2509 (0.7); 7.2484 (1.0); 6.9970 (0.6); 6.9201 (0.8); 6.9186 (0.9); 6.9126 (0.9);
6.9111 (0.9); 6.8990 (0.8); 6.8975 (0.8); 6.8915 (0.8); 6.8900 (0.8); 4.9056 (8.5); 3.7858 (16.0); 1.5980 (0.8); 1.5811 (1.2); 1.5639
(1.2); 1.5473 (3.0); 0.7823 (10.1); 0.7652 (6.5); 0.0079 (1.9); −0.0002 (66.1); −0.0085 (2.0)
I-43: ¹H-NMR(400.6 MHz, CDCl3):
δ = 8.2723 (1.8); 8.2623 (1.8); 8.2607 (1.8); 8.1385 (2.7); 8.1324 (2.8); 7.7709 (1.1); 7.7647 (1.1); 7.7519 (1.4); 7.7499 (1.5);
7.7457 (1.4); 7.7437 (1.5); 7.7309 (1.2); 7.7247 (1.2); 7.4642 (1.1); 7.4607 (1.1); 7.4435 (1.6); 7.4404 (2.2); 7.4375 (1.4); 7.4203
(1.4); 7.4168 (1.4); 7.3098 (1.2); 7.3007 (1.5); 7.2984 (1.4); 7.2892 (2.1); 7.2801 (1.1); 7.2778 (1.2); 7.2686 (1.0); 7.2626 (34.1);
6.9256 (1.7); 6.9193 (1.7); 6.9044 (1.7); 6.8982 (1.6); 5.3001 (1.1); 4.9419 (16.0); 1.6072 (0.5); 1.5933 (1.0); 1.5867 (1.1); 1.5809
(0.7); 1.5731 (2.0); 1.5669 (0.8); 1.5590 (1.0); 1.5526 (1.1); 1.5389 (0.6); 1.4320 (1.2); 1.2644 (1.6); 0.8986 (0.9); 0.8817 (3.3);
0.8640 (1.2); 0.7961 (0.6); 0.7808 (1.8); 0.7762 (3.1); 0.7734 (2.1); 0.7697 (2.5); 0.7601 (2.0); 0.7574 (2.2); 0.7532 (4.2); 0.7495
(4.2); 0.7462 (3.1); 0.7386 (3.0); 0.7341 (3.6); 0.7312 (2.2); 0.7282 (2.1); 0.0079 (0.5); −0.0002 (20.9); −0.0085 (0.7)
I-44: ¹H-NMR(400.6 MHz, CDCl3):
δ = 8.2886 (0.9); 8.2869 (1.0); 8.2850 (1.1); 8.2834 (1.0); 8.2770 (1.0); 8.2753 (1.1); 8.2734 (1.1); 8.2718 (1.0); 8.1543 (1.4);
8.1480 (1.5); 7.8684 (0.7); 7.8622 (0.7); 7.8499 (0.8); 7.8472 (0.9); 7.8437 (0.8); 7.8410 (0.9); 7.8287 (0.8); 7.8224 (0.8); 7.5556
(0.7); 7.5519 (0.8); 7.5348 (1.0); 7.5312 (1.2); 7.5290 (0.9); 7.5119 (1.0); 7.5082 (1.0); 7.4147 (1.0); 7.4055 (1.1); 7.4031 (1.0);
7.3940 (1.7); 7.3848 (0.8); 7.3824 (0.8); 7.3732 (0.7); 7.2618 (17.1); 6.9914 (0.9); 6.9900 (1.0); 6.9840 (1.0); 6.9825 (1.0); 6.9702
(0.9); 6.9687 (1.0); 6.9627 (1.0); 6.9613 (1.0); 4.9522 (11.5); 3.3771 (16.0); 3.3705 (0.8); 2.1725 (3.9); 0.8819 (0.7); 0.0080
(0.6); −0.0002 (21.4); −0.0085 (0.7)
I-45: ¹H-NMR(400.6 MHz, CDCl3):
δ = 8.2753 (0.8); 8.2734 (0.8); 8.2637 (0.8); 8.2618 (0.8); 8.1311 (1.1); 8.1249 (1.2); 7.8640 (0.5); 7.8578 (0.5); 7.8452 (0.6);
7.8428 (0.7); 7.8390 (0.6); 7.8366 (0.6); 7.8241 (0.6); 7.8179 (0.6); 7.5011 (0.5); 7.4975 (0.5); 7.4804 (0.8); 7.4773 (0.9); 7.4747
(0.6); 7.4575 (0.7); 7.4538 (0.7); 7.3653 (0.7); 7.3562 (0.8); 7.3538 (0.7); 7.3446 (1.2); 7.3355 (0.6); 7.3331 (0.6); 7.2609 (29.4);
6.9670 (0.8); 6.9604 (0.8); 6.9466 (0.7); 6.9392 (0.7); 5.1198 (8.6); 2.0433 (14.2); 1.9725 (15.2); 1.5477 (16.0); 1.2599 (0.7);
0.0081 (1.0); −0.0002 (34.2); −0.0085 (1.2)
I-46: ¹H-NMR(400.6 MHz, CDCl3):
δ = 8.5752 (1.4); 8.5650 (1.4); 8.5631 (1.4); 8.2657 (1.6); 8.2639 (1.7); 8.2542 (1.7); 8.2523 (1.7); 8.1326 (2.2); 8.1263 (2.4);
7.8629 (1.1); 7.8567 (1.1); 7.8441 (1.3); 7.8418 (1.4); 7.8379 (1.3); 7.8355 (1.3); 7.8230 (1.2); 7.8168 (1.2); 7.6351 (1.0); 7.6306
(1.0); 7.6158 (2.0); 7.6114 (2.0); 7.5966 (1.2); 7.5921 (1.2); 7.5121 (1.0); 7.5084 (1.1); 7.4913 (1.6); 7.4882 (1.8); 7.4854 (1.2);
7.4684 (1.4); 7.4647 (1.4); 7.3682 (1.5); 7.3592 (3.6); 7.3570 (2.7); 7.3475 (2.6); 7.3387 (2.6); 7.3269 (1.1); 7.2609 (39.1); 7.2238
(1.1); 7.2115 (1.1); 7.2049 (1.1); 7.1927 (1.0); 6.9673 (1.6); 6.9599 (1.6); 6.9461 (1.5); 6.9387 (1.6); 5.3574 (11.4); 5.0698 (16.0);
2.1719 (0.9); 1.5601 (2.8); 1.2597 (0.6); 0.8819 (0.9); 0.0078 (1.5); 0.0053 (0.6); −0.0002 (53.1); −0.0085 (1.9)
I-47: ¹H-NMR(400.6 MHz, CDCl3):
δ = 8.2893 (1.8); 8.2875 (1.8); 8.2778 (1.8); 8.2759 (1.8); 8.1551 (2.4); 8.1488 (2.6); 7.8508 (1.1); 7.8446 (1.1); 7.8321 (1.3);
7.8297 (1.4); 7.8259 (1.4); 7.8235 (1.4); 7.8110 (1.2); 7.8048 (1.2); 7.5321 (1.1); 7.5285 (1.1); 7.5113 (1.6); 7.5085 (1.9); 7.4886
(1.5); 7.4849 (1.5); 7.3914 (1.4); 7.3822 (1.6); 7.3798 (1.5); 7.3707 (2.5); 7.3615 (1.2); 7.3591 (1.2); 7.3500 (1.0); 7.2631 (14.3);
7.1166 (0.8); 6.9713 (1.7); 6.9640 (1.8); 6.9502 (1.7); 6.9428 (1.7); 4.8520 (15.0); 4.2002 (2.2); 4.1824 (6.8); 4.1646 (6.9); 4.1468
(2.3); 3.6798 (1.4); 3.6644 (3.8); 3.6492 (4.0); 3.6337 (1.6); 2.6179 (3.4); 2.6025 (5.4); 2.5874 (3.3); 1.5857 (5.6); 1.2918 (7.8);
1.2740 (16.0); 1.2562 (8.0); 0.0080 (0.6); −0.0002 (20.9); −0.0085 (0.8)
I-48: ¹H-NMR(400.6 MHz, CDCl3):
δ = 8.2969 (1.4); 8.2953 (1.6); 8.2934 (1.7); 8.2853 (1.6); 8.2837 (1.8); 8.2818 (1.7); 8.1577 (2.2); 8.1514 (2.4); 7.8583 (1.1);
7.8520 (1.1); 7.8396 (1.3); 7.8371 (1.4); 7.8334 (1.3); 7.8308 (1.3); 7.8184 (1.2); 7.8122 (1.2); 7.5328 (1.0); 7.5291 (1.1); 7.5120
(1.6); 7.5086 (1.8); 7.5064 (1.3); 7.4892 (1.5); 7.4855 (1.4); 7.3936 (1.4); 7.3844 (1.6); 7.3820 (1.5); 7.3729 (2.4); 7.3637 (1.1);
7.3613 (1.2); 7.3522 (1.0); 7.2631 (13.4); 7.1160 (0.8); 6.9741 (1.6); 6.9730 (1.6); 6.9667 (1.6); 6.9530 (1.6); 6.9518 (1.5); 6.9455
(1.6); 4.9173 (13.2); 4.2839 (2.0); 4.2661 (6.3); 4.2482 (6.4); 4.2304 (2.1); 4.1605 (6.3); 4.1474 (6.4); 4.1309 (0.9); 4.1131 (0.8);
2.0454 (3.7); 1.5810 (4.7); 1.3269 (7.8); 1.3091 (16.0); 1.2913 (7.9); 1.2774 (1.3); 1.2596 (2.6); 1.2417 (1.1); 0.8819 (1.2); 0.8642
(0.5); 0.0080 (0.5); −0.0002 (19.8); −0.0085 (0.7)
I-49: ¹H-NMR(400.6 MHz, CDCl3):
δ = 8.3109 (0.7); 8.3091 (0.8); 8.3072 (0.8); 8.3056 (0.7); 8.2992 (0.7); 8.2976 (0.8); 8.2956 (0.8); 8.2941 (0.7); 8.1055 (1.3);
8.1038 (1.3); 8.0994 (1.4); 8.0977 (1.3); 7.5663 (1.3); 7.5602 (1.2); 7.5448 (1.3); 7.5386 (1.3); 7.4810 (0.5); 7.4772 (0.5); 7.4603
(0.8); 7.4581 (0.7); 7.4567 (0.8); 7.4546 (0.6); 7.4376 (0.7); 7.4338 (0.7); 7.3518 (0.7); 7.3427 (0.8); 7.3402 (0.8); 7.3311 (1.2);
7.3220 (0.5); 7.3196 (0.6); 7.2607 (18.0); 6.7239 (1.5); 6.7220 (1.6); 6.7023 (1.5); 6.7005 (1.5); 4.9554 (7.4); 3.9244 (16.0);
3.7958 (13.8); 2.0453 (1.4); 1.5491 (6.9); 1.2597 (1.0); 0.8821 (0.7); 0.0079 (0.8); −0.0002 (27.1); −0.0085 (0.8)
I-50: ¹H-NMR(400.6 MHz, d₆-DMSO):
δ = 12.9883 (1.4); 8.3151 (0.8); 8.3128 (1.1); 8.3098 (0.9); 8.3035 (0.9); 8.3012 (1.2); 8.2983 (0.9); 8.1041 (1.7); 8.1024 (1.7);
8.0979 (1.8); 8.0962 (1.8); 7.9660 (0.6); 7.9625 (0.7); 7.9451 (0.8); 7.9415 (1.4); 7.9378 (0.8); 7.9205 (0.8); 7.9169 (0.8); 7.6123
(0.8); 7.6028 (1.0); 7.6007 (0.9); 7.5968 (1.7); 7.5909 (2.5); 7.5819 (0.8); 7.5799 (0.8); 7.5752 (1.8); 7.5690 (1.9); 6.8624 (1.9);
6.8606 (1.9); 6.8408 (1.9); 6.8390 (1.9); 5.7571 (0.6); 5.0423 (1.8); 5.0248 (1.9); 3.8484 (16.0); 3.3250 (4.9); 2.5199 (0.6); 2.5112
(8.7); 2.5066 (19.2); 2.5020 (27.1); 2.4975 (19.6); 2.4930 (9.6); 2.4707 (0.6); 2.0859 (1.4); 1.5681 (5.0); 1.5507 (5.0); 0.0080
(0.5); −0.0002 (17.6); −0.0085 (0.7)
I-51: ¹H-NMR(400.6 MHz, CDCl3):
δ = 8.4517 (2.9); 8.4448 (3.0); 8.3015 (1.7); 8.2977 (3.0); 8.2941 (1.7); 8.2482 (1.3); 8.2464 (1.5); 8.2445 (1.6); 8.2429 (1.4);
8.2365 (1.4); 8.2348 (1.6); 8.2329 (1.5); 8.2314 (1.3); 7.5287 (1.3); 7.5243 (2.4); 7.5216 (1.8); 7.5175 (1.3); 7.5065 (1.4); 7.5034
(1.9); 7.4998 (2.9); 7.4974 (1.5); 7.4953 (1.4); 7.4805 (1.4); 7.4768 (1.4); 7.3609 (1.4); 7.3518 (1.5); 7.3493 (1.4); 7.3402 (2.4);
7.3312 (1.1); 7.3286 (1.1); 7.3196 (1.0); 7.2637 (7.8); 5.3004 (1.5); 4.9389 (15.7); 4.2929 (1.9); 4.2751 (6.1); 4.2572 (6.2); 4.2394
(2.0); 1.6008 (1.7); 1.3049 (7.7); 1.2871 (16.0); 1.2693 (7.5); −0.0002 (11.3)
I-52: ¹H-NMR(400.6 MHz, CDCl3):
δ = 8.5510 (1.8); 8.5487 (2.0); 8.5467 (2.0); 8.5444 (1.7); 8.5390 (1.8); 8.5367 (2.1); 8.5345 (2.0); 8.5324 (1.6); 8.2331 (2.3);
8.2313 (2.5); 8.2294 (2.5); 8.2278 (2.1); 8.2214 (2.3); 8.2197 (2.6); 8.2178 (2.5); 8.1223 (3.2); 8.1161 (3.3); 7.8536 (1.6); 7.8473
(1.5); 7.8348 (2.0); 7.8324 (2.0); 7.8286 (1.9); 7.8262 (1.8); 7.8137 (1.6); 7.8074 (1.6); 7.5612 (1.2); 7.5567 (1.2); 7.5419 (2.6);
7.5375 (2.6); 7.5226 (1.7); 7.5185 (1.8); 7.5036 (1.6); 7.4999 (1.6); 7.4829 (2.4); 7.4795 (2.6); 7.4767 (1.8); 7.4598 (2.1); 7.4561
(2.0); 7.3511 (2.0); 7.3421 (2.5); 7.3395 (2.1); 7.3305 (3.8); 7.3216 (3.5); 7.3190 (2.1); 7.3098 (1.8); 7.3045 (2.4); 7.2605 (83.7);
7.1889 (1.5); 7.1756 (1.5); 7.1702 (1.5); 7.1581 (1.4); 6.9607 (2.3); 6.9533 (2.4); 6.9518 (2.1); 6.9395 (2.2); 6.9380 (2.1); 6.9320
(2.2); 6.9305 (1.9); 5.4288 (1.1); 5.4114 (4.3); 5.3940 (4.4); 5.3767 (1.1); 5.3338 (13.9); 1.7655 (16.0); 1.7481 (15.8); 1.5507
(10.8); 0.0079 (3.8); −0.0002 (129.1); −0.0061 (1.9); −0.0085 (3.8)
I-53: ¹H-NMR(400.6 MHz, CDCl3):
δ = 8.6885 (0.9); 8.2923 (0.8); 8.2907 (0.9); 8.2888 (1.0); 8.2873 (0.9); 8.2808 (0.9); 8.2791 (1.0); 8.2772 (1.0); 8.1466 (1.3);
8.1404 (1.4); 7.8679 (0.6); 7.8616 (0.6); 7.8493 (0.7); 7.8467 (0.8); 7.8431 (0.8); 7.8405 (0.8); 7.8281 (0.7); 7.8218 (0.7); 7.5445
(0.6); 7.5409 (0.6); 7.5237 (1.0); 7.5201 (1.1); 7.5182 (0.8); 7.5010 (0.9); 7.4973 (0.8); 7.4078 (0.8); 7.3987 (1.0); 7.3963 (0.9);
7.3871 (1.4); 7.3779 (0.7); 7.3755 (0.7); 7.3664 (0.6); 7.2613 (15.8); 6.9881 (0.8); 6.9868 (0.9); 6.9806 (0.9); 6.9793 (0.9); 6.9669
(0.8); 6.9655 (0.9); 6.9595 (0.9); 6.9581 (0.9); 4.8859 (9.7); 4.2566 (0.8); 4.2398 (1.2); 4.2230 (0.9); 2.9082 (16.0); 2.0455 (0.6);
1.5543 (8.7); 1.2596 (0.6); 1.1572 (13.6); 1.1404 (13.6); 0.8819 (0.5); 0.0080 (0.7); −0.0002 (24.3); −0.0085 (1.0)
I-54: ¹H-NMR(400.0 MHz, CDCl3):
δ = 9.1339 (2.0); 8.2860 (2.0); 8.2746 (2.0); 8.1517 (2.7); 8.1460 (2.7); 7.8574 (1.0); 7.8512 (1.0); 7.8362 (1.5); 7.8302 (1.4);
7.8175 (1.1); 7.8112 (1.0); 7.5408 (0.9); 7.5200 (2.3); 7.4967 (1.1); 7.4053 (1.1); 7.3942 (1.6); 7.3846 (2.0); 7.3734 (1.2); 7.3641
(0.8); 7.2612 (74.0); 6.9805 (1.8); 6.9731 (1.8); 6.9592 (1.8); 6.9519 (1.8); 5.3003 (3.1); 4.9221 (13.0); 4.2494 (1.5); 4.2316 (4.7);
4.2138 (4.8); 4.1960 (1.7); 4.1761 (9.3); 4.1311 (0.8); 4.1130 (0.8); 3.0726 (16.0); 2.0451 (3.6); 1.5673 (13.2); 1.3048 (5.6);
1.2869 (11.3); 1.2775 (1.8); 1.2690 (6.4); 1.2596 (4.0); 1.2418 (1.1); 0.8982 (0.6); 0.8818 (1.3); 0.8645 (0.7); 0.0080 (1.2); −0.0002
(39.5); −0.0084 (1.2)
I-55: ¹H-NMR(400.0 MHz, CDCl3):
δ = 8.2454 (1.1); 8.2356 (1.1); 8.1358 (1.4); 8.1295 (1.5); 7.8564 (0.7); 7.8502 (0.6); 7.8354 (0.9); 7.8314 (0.8); 7.8292 (0.8);
7.8165 (0.7); 7.8102 (0.7); 7.4959 (0.6); 7.4922 (0.6); 7.4752 (0.9); 7.4718 (1.2); 7.4522 (0.9); 7.4484 (0.8); 7.3429 (0.8); 7.3339
(1.0); 7.3314 (0.9); 7.3223 (1.5); 7.3131 (0.7); 7.3106 (0.7); 7.3017 (0.6); 7.2612 (42.3); 6.9602 (1.0); 6.9528 (1.0); 6.9390 (1.0);
6.9315 (0.9); 4.9300 (9.9); 4.2871 (1.2); 4.2692 (3.8); 4.2513 (3.8); 4.2335 (1.2); 1.5513 (16.0); 1.3026 (4.5); 1.2848 (9.2); 1.2669
(4.5); 0.0079 (0.8); −0.0002 (26.3); −0.0086 (0.7)
I-56: ¹H-NMR(400.6 MHz, CDCl3):
δ = 8.2591 (1.4); 8.2572 (1.4); 8.2475 (1.5); 8.2457 (1.5); 8.1173 (1.9); 8.1113 (2.0); 7.8362 (0.7); 7.8300 (0.7); 7.8156 (1.0);
7.8100 (1.0); 7.7965 (0.7); 7.7904 (0.7); 7.5148 (1.0); 7.5111 (1.0); 7.4940 (1.6); 7.4922 (1.5); 7.4903 (1.7); 7.4887 (1.3); 7.4715
(1.5); 7.4678 (1.4); 7.3878 (1.3); 7.3785 (1.5); 7.3763 (1.4); 7.3670 (2.1); 7.3577 (1.0); 7.3555 (1.0); 7.3462 (0.9); 7.2663 (4.6);
6.9493 (1.5); 6.9478 (1.5); 6.9418 (1.6); 6.9404 (1.5); 6.9281 (1.4); 6.9266 (1.4); 6.9207 (1.4); 6.9192 (1.4); 4.9394 (13.9); 4.2846
(2.0); 4.2667 (6.3); 4.2489 (6.3); 4.2311 (2.1); 3.8782 (0.5); 3.0300 (0.5); 2.9983 (0.5); 1.6151 (4.8); 1.3027 (0.7); 1.2975 (7.8);
1.2850 (1.6); 1.2797 (16.0); 1.2671 (0.8); 1.2619 (7.7); −0.0002 (6.3)
I-57: ¹H-NMR(400.6 MHz, CDCl3):
δ = 8.2960 (1.2); 8.2940 (1.2); 8.2845 (1.2); 8.2825 (1.2); 8.1270 (1.6); 8.1209 (1.6); 7.9824 (0.7); 7.9762 (0.7); 7.9639 (0.8);
7.9611 (0.9); 7.9577 (0.8); 7.9550 (0.8); 7.9427 (0.7); 7.9365 (0.7); 7.5492 (0.7); 7.5455 (0.7); 7.5281 (1.3); 7.5245 (1.3); 7.5061
(1.0); 7.5025 (1.0); 7.4495 (1.0); 7.4400 (1.2); 7.4381 (1.1); 7.4286 (1.6); 7.4191 (0.8); 7.4172 (0.8); 7.4078 (0.6); 7.2621 (11.3);
7.0078 (1.1); 7.0068 (1.1); 7.0005 (1.1); 6.9865 (1.0); 6.9854 (1.0); 6.9792 (1.1); 5.0231 (10.7); 4.2976 (1.4); 4.2797 (4.2); 4.2619
(4.3); 4.2441 (1.4); 2.0457 (0.9); 1.5588 (16.0); 1.3018 (5.0); 1.2840 (10.2); 1.2777 (0.6); 1.2661 (5.2); 1.2598 (0.9); 0.8819 (0.5);
0.0079 (0.6); −0.0002 (17.1); −0.0085 (0.6)
I-59: ¹H-NMR(400.6 MHz, CDCl3):
δ = 8.2170 (1.3); 8.2109 (1.4); 8.0929 (0.7); 8.0831 (0.7); 7.8722 (0.6); 7.8661 (0.6); 7.8533 (0.7); 7.8512 (0.8); 7.8471 (0.7);
7.8450 (0.7); 7.8324 (0.7); 7.8262 (0.6); 7.8117 (0.5); 7.8071 (0.5); 7.7930 (0.7); 7.7910 (0.9); 7.7886 (0.8); 7.7865 (0.9); 7.7727
(0.8); 7.7681 (0.8); 7.7016 (1.2); 7.6810 (0.8); 7.2675 (2.8); 7.1558 (0.7); 7.1536 (0.7); 7.1437 (0.7); 7.1414 (0.7); 7.1376 (0.7);
7.1353 (0.6); 7.1254 (0.6); 7.1231 (0.6); 7.0153 (0.9); 7.0082 (0.9); 6.9942 (0.8); 6.9871 (0.9); 4.9144 (7.8); 4.7249 (0.9); 4.7068
(1.3); 4.6887 (0.9); 3.7815 (16.0); 2.0093 (0.8); 1.5140 (6.5); 1.4961 (6.5); −0.0002 (3.4)
I-60: ¹H-NMR(400.6 MHz, CDCl3):
δ = 8.2130 (2.0); 8.2068 (2.2); 8.1268 (1.3); 8.1248 (1.3); 8.1222 (1.5); 8.1146 (1.4); 8.1100 (1.4); 7.8671 (1.0); 7.8609 (1.1);
7.8483 (1.3); 7.8460 (1.3); 7.8421 (1.3); 7.8398 (1.2); 7.8273 (1.1); 7.8222 (1.5); 7.8178 (1.2); 7.8019 (1.5); 7.7991 (1.4); 7.7972
(1.4); 7.7833 (1.5); 7.7786 (1.4); 7.6286 (2.7); 7.6081 (2.2); 7.5188 (0.9); 7.2603 (167.8); 7.1912 (1.4); 7.1887 (1.3); 7.1790 (1.4);
7.1765 (1.3); 7.1727 (1.3); 7.1702 (1.2); 7.1605 (1.3); 7.1580 (1.2); 7.0254 (1.4); 7.0182 (1.4); 7.0044 (1.5); 6.9968 (2.4); 5.0298
(0.7); 4.9840 (16.0); 4.3501 (1.6); 4.3287 (5.1); 4.3073 (5.3); 4.2859 (1.8); 1.6115 (4.3); 1.2538 (0.6); 0.1457 (0.7); 0.0080
(5.1); −0.0002 (208.2); −0.0085 (7.5); −0.0294 (0.6); −0.1494 (0.7)
I-61: ¹H-NMR(400.6 MHz, CDCl3):
δ = 8.2552 (1.8); 8.2422 (1.8); 8.0907 (0.9); 8.0881 (1.0); 8.0806 (0.9); 8.0786 (1.0); 8.0760 (1.0); 7.8361 (0.7); 7.8315 (0.6);
7.8176 (0.9); 7.8155 (1.1); 7.8129 (1.0); 7.8109 (1.0); 7.7971 (1.0); 7.7925 (1.0); 7.7156 (1.9); 7.6950 (1.3); 7.2606 (43.7); 7.1807
(0.9); 7.1781 (0.9); 7.1685 (0.9); 7.1660 (0.9); 7.1623 (1.0); 7.1598 (0.8); 7.1501 (0.9); 7.1475 (0.8); 7.1328 (0.8); 7.1293 (1.2);
7.1251 (0.9); 7.1199 (0.9); 7.1163 (1.2); 7.1122 (0.9); 6.9434 (1.9); 5.3003 (2.5); 4.9419 (8.0); 4.1829 (3.7); 4.1696 (3.7); 3.7948
(16.0); 1.5424 (16.0); 1.2556 (1.2); 0.0079 (2.2); −0.0002 (70.3); −0.0085 (2.7)
I-62: ¹H-NMR(400.6 MHz, d₆-DMSO):
δ = 12.3088 (1.1); 8.2504 (2.8); 8.2443 (2.9); 8.1007 (1.7); 8.0988 (1.9); 8.0962 (2.0); 8.0941 (1.8); 8.0887 (1.8); 8.0867 (2.1);
8.0840 (2.0); 8.0820 (1.8); 8.0231 (1.2); 8.0169 (1.1); 8.0020 (1.8); 7.9958 (1.7); 7.9824 (1.3); 7.9762 (1.2); 7.9594 (1.5); 7.9547
(1.4); 7.9409 (1.8); 7.9388 (2.0); 7.9362 (1.8); 7.9341 (1.8); 7.9203 (1.8); 7.9155 (1.7); 7.6788 (2.0); 7.6766 (3.6); 7.6746 (2.4);
7.6582 (1.8); 7.6561 (3.2); 7.6539 (2.0); 7.3018 (1.9); 7.2960 (2.0); 7.2803 (2.1); 7.2785 (2.5); 7.2757 (3.0); 7.2663 (1.8); 7.2639
(2.0); 7.2599 (1.8); 7.2575 (1.6); 7.2478 (1.7); 7.2453 (1.7); 5.1825 (0.6); 5.1460 (2.1); 5.0933 (2.0); 5.0579 (0.6); 4.2193 (0.6);
4.1869 (0.6); 4.0986 (0.8); 4.0808 (0.8); 4.0557 (1.1); 4.0379 (3.4); 4.0201 (3.4); 4.0024 (1.2); 3.8076 (0.6); 3.7738 (0.6); 3.3220
(30.6); 3.1522 (0.7); 2.7920 (0.7); 2.6705 (0.5); 2.5680 (0.6); 2.5583 (1.1); 2.5484 (0.7); 2.5411 (2.1); 2.5301 (0.8); 2.5243 (1.4);
2.5197 (1.8); 2.5108 (24.5); 2.5063 (54.7); 2.5017 (77.4); 2.4971 (54.8); 2.4926 (25.3); 2.1832 (0.6); 1.9887 (16.0); 1.9090 (1.5);
1.8718 (0.9); 1.8258 (0.7); 1.6402 (0.5); 1.4102 (0.5); 1.3554 (5.2); 1.2351 (0.9); 1.2036 (1.0); 1.1922 (4.7); 1.1858 (2.3); 1.1745
(9.3); 1.1681 (1.1); 1.1567 (4.5); 0.0080 (1.3); −0.0002 (48.1); −0.0085 (1.5)
I-63: ¹H-NMR(400.6 MHz, d₆-DMSO):
δ = 12.5889 (1.3); 8.2481 (6.1); 8.1032 (4.6); 8.0943 (4.5); 8.0215 (2.1); 8.0155 (2.2); 8.0009 (3.9); 7.9959 (3.8); 7.9808 (3.6);
7.9753 (3.6); 7.9595 (3.1); 7.7000 (3.0); 7.6799 (5.5); 7.6599 (2.8); 7.3025 (4.1); 7.2964 (4.3); 7.2822 (5.5); 7.2748 (7.1); 7.2566
(3.1); 6.8699 (0.6); 5.0144 (10.4); 4.9638 (0.7); 4.0552 (1.3); 4.0378 (3.7); 4.0200 (3.7); 4.0024 (1.3); 3.7947 (1.0); 3.7698 (1.8);
3.7501 (1.5); 3.6783 (1.6); 3.6607 (1.9); 3.6348 (1.6); 3.6097 (1.8); 3.5937 (2.7); 3.5751 (2.6); 3.5638 (4.3); 3.5448 (5.4); 3.5291
(2.7); 3.4985 (1.2); 3.4675 (1.4); 3.4491 (1.4); 3.4370 (1.0); 3.3927 (1.2); 3.3741 (2.3); 3.3206 (26.0); 3.2046 (1.4); 3.1859 (1.8);
3.1672 (1.3); 3.0778 (1.4); 3.0598 (1.9); 3.0420 (1.4); 2.6701 (1.6); 2.5404 (6.6); 2.5014 (226.7); 2.3286 (1.6); 2.2051 (1.2);
2.1850 (2.1); 2.1719 (1.6); 2.1444 (1.6); 2.1259 (1.8); 2.1129 (1.7); 2.0981 (1.7); 2.0809 (1.5); 2.0677 (1.2); 1.9886 (16.0); 1.9701
(1.8); 1.9576 (1.2); 1.9375 (0.9); 1.9087 (0.6); 1.7602 (0.6); 1.3551 (7.0); 1.2352 (3.8); 1.1921 (4.2); 1.1744 (8.0); 1.1565
(4.1); −0.0002 (65.7)
I-64: ¹H-NMR(400.6 MHz, CDCl3):
δ = 8.2218 (0.8); 8.2198 (1.2); 8.2178 (0.9); 8.2157 (0.9); 8.2136 (1.2); 8.2117 (0.8); 8.0975 (0.7); 8.0954 (0.8); 8.0928 (0.8);
8.0907 (0.8); 8.0854 (0.8); 8.0832 (0.8); 8.0807 (0.9); 8.0786 (0.8); 7.8676 (0.7); 7.8613 (0.7); 7.8487 (0.8); 7.8464 (0.8); 7.8426
(0.8); 7.8402 (0.8); 7.8276 (0.7); 7.8214 (0.7); 7.8129 (0.7); 7.8082 (0.6); 7.7946 (0.7); 7.7922 (1.0); 7.7899 (0.8); 7.7875 (1.0);
7.7739 (1.1); 7.7692 (1.0); 7.7117 (1.1); 7.7093 (1.8); 7.7069 (1.1); 7.6910 (0.7); 7.6887 (1.2); 7.6863 (0.7); 7.2624 (8.4); 7.1545
(0.9); 7.1519 (0.9); 7.1424 (0.8); 7.1397 (0.9); 7.1362 (0.9); 7.1335 (0.8); 7.1241 (0.9); 7.1214 (0.8); 7.0113 (0.9); 7.0097 (0.9);
7.0039 (0.9); 7.0023 (0.9); 6.9901 (0.8); 6.9885 (0.9); 6.9827 (0.8); 6.9811 (0.8); 5.3003 (3.8); 4.9474 (7.2); 4.1834 (3.2); 4.1699
(3.2); 3.7905 (16.0); 1.5748 (1.9); −0.0002 (13.5)
I-65: ¹H-NMR(400.6 MHz, CDCl3):
δ = 8.5021 (1.2); 8.4957 (1.3); 8.3435 (1.5); 8.1153 (1.2); 8.1122 (0.8); 8.1066 (0.8); 8.1034 (1.4); 8.1001 (0.8); 7.8413 (1.4);
7.8377 (2.5); 7.8293 (3.0); 7.8259 (2.5); 7.5117 (0.7); 7.5049 (0.8); 7.5006 (0.7); 7.4894 (0.7); 7.4852 (0.8); 7.4826 (0.8); 7.4783
(0.7); 7.2603 (46.8); 7.2092 (0.9); 7.1972 (1.4); 7.1867 (1.4); 7.1759 (0.8); 4.0858 (4.1); 4.0727 (4.2); 3.8973 (8.2); 3.7025 (16.0);
2.0455 (0.5); 1.5432 (6.4); 0.0081 (1.9); −0.0002 (73.8); −0.0085 (2.8)
I-66: ¹H-NMR(400.6 MHz, d₆-DMSO):
δ = 13.1115 (0.5); 8.2587 (3.0); 8.2525 (3.3); 8.1165 (2.0); 8.1144 (2.3); 8.1118 (2.6); 8.1097 (2.3); 8.1044 (2.2); 8.1023 (2.5);
8.0997 (2.5); 8.0976 (2.4); 8.0300 (1.5); 8.0238 (1.4); 8.0103 (1.9); 8.0089 (2.1); 8.0042 (1.9); 8.0027 (2.0); 7.9916 (2.3); 7.9893
(2.0); 7.9869 (2.3); 7.9830 (1.7); 7.9731 (2.4); 7.9710 (2.5); 7.9683 (2.3); 7.9663 (2.4); 7.9525 (2.5); 7.9477 (2.4); 7.6838 (2.4);
7.6816 (4.6); 7.6794 (2.7); 7.6632 (2.3); 7.6610 (4.2); 7.6587 (2.4); 7.3033 (2.1); 7.2962 (4.4); 7.2937 (2.8); 7.2838 (3.8); 7.2817
(4.2); 7.2774 (3.5); 7.2751 (4.2); 7.2655 (2.4); 7.2630 (2.2); 4.9230 (16.0); 4.0377 (1.5); 4.0199 (1.5); 4.0021 (0.5); 3.3209 (47.8);
2.6746 (0.8); 2.6700 (1.1); 2.6655 (0.8); 2.5406 (1.4); 2.5238 (2.0); 2.5191 (3.0); 2.5104 (56.8); 2.5058 (127.4); 2.5012 (181.4);
2.4966 (126.9); 2.4920 (57.9); 2.4748 (1.1); 2.4708 (0.6); 2.3329 (0.8); 2.3282 (1.1); 2.3236 (0.8); 1.9885 (7.1); 1.9084 (3.9);
1.1921 (2.1); 1.1744 (4.4); 1.1566 (2.1); 0.1457 (0.6); 0.0102 (0.5); 0.0080 (5.2); 0.0063 (1.1); 0.0055 (1.2); 0.0046 (1.5); 0.0038
(2.0); −0.0002 (189.9); −0.0027 (9.5); −0.0043 (4.1); −0.0051 (3.0); −0.0060 (2.5); −0.0068 (2.1); −0.0085 (5.8); −0.0107 (1.1); −0.0115
(0.9); −0.0123 (0.8); −0.0131 (0.7); −0.0139 (0.5); −0.0274 (0.7); −0.1494 (0.6)
I-67: ¹H-NMR(400.0 MHz, CDCl3):
δ = 9.2223 (4.2); 8.7933 (9.7); 8.0386 (1.1); 8.0292 (1.1); 7.7968 (0.6); 7.7920 (0.6); 7.7751 (1.2); 7.7578 (1.2); 7.7530 (1.1);
7.7132 (2.1); 7.6926 (1.2); 7.2614 (56.4); 7.1244 (1.0); 7.1098 (1.1); 7.1066 (1.0); 7.0941 (0.8); 4.9797 (9.5); 4.9559 (0.6); 3.8404
(16.0); 3.8303 (1.3); 1.6019 (1.0); 1.4373 (0.5); 1.2539 (1.8); 0.0073 (2.0); −0.0002 (32.8); −0.0084 (1.5)
I-68: ¹H-NMR(599.6 MHz, CDCl3):
δ = 9.2245 (0.3); 8.7897 (0.7); 8.6074 (0.4); 7.2712 (3.2); 4.9556 (0.7); 4.3109 (0.3); 4.2995 (0.3); 2.7549 (23.2); 2.6273 (50.0);
2.0446 (0.3); 2.0335 (0.4); 1.8407 (0.6); 1.3738 (0.3); 1.3334 (2.0); 1.3234 (1.1); 1.3110 (1.1); 1.2849 (3.2); 1.2567 (12.3); 0.8911
(0.8); 0.8806 (1.4); 0.8690 (0.8); 0.8422 (0.4); 0.0699 (1.8); −0.0001 (4.0)
I-69: ¹H-NMR(400.6 MHz, d₆-DMSO):
δ = 12.8847 (1.2); 8.6620 (4.8); 8.6550 (4.9); 8.4145 (2.5); 8.4102 (4.7); 8.4068 (2.3); 8.1432 (1.8); 8.1411 (2.0); 8.1386 (2.3);
8.1365 (2.1); 8.1311 (2.0); 8.1291 (2.2); 8.1264 (2.3); 8.1244 (2.1); 8.0475 (1.7); 8.0428 (1.6); 8.0289 (2.1); 8.0269 (2.2); 8.0242
(1.9); 8.0223 (2.1); 8.0084 (2.2); 8.0037 (2.1); 7.8809 (1.8); 7.8767 (1.8); 7.8740 (1.8); 7.8697 (1.7); 7.8569 (1.9); 7.8527 (2.0);
7.8499 (1.8); 7.8457 (1.7); 7.8140 (2.3); 7.8117 (4.1); 7.8095 (2.4); 7.7935 (2.1); 7.7912 (3.6); 7.7889 (2.0); 7.3571 (2.1); 7.3546
(2.1); 7.3450 (1.9); 7.3425 (2.1); 7.3385 (2.1); 7.3360 (1.9); 7.3264 (2.0); 7.3239 (2.0); 5.7566 (9.1); 4.0556 (0.7); 4.0351 (16.0);
4.0201 (2.0); 4.0023 (0.7); 3.3231 (51.0); 2.6703 (0.7); 2.5409 (1.2); 2.5240 (1.5); 2.5194 (2.3); 2.5107 (35.4); 2.5061 (77.7);
2.5015 (108.4); 2.4969 (74.2); 2.4924 (32.7); 2.4782 (0.6); 2.4738 (0.5); 2.3286 (0.6); 1.9886 (8.6); 1.1922 (2.6); 1.1745 (5.2);
1.1567 (2.6); 0.0080 (2.7); 0.0061 (0.6); −0.0002 (103.1); −0.0044 (1.4); −0.0053 (1.0); −0.0061 (0.9); −0.0070 (0.8); −0.0085 (2.9)
I-70: ¹H-NMR(400.6 MHz, CDCl3):
δ = 8.2098 (1.4); 8.2076 (1.1); 8.2056 (1.2); 8.2037 (1.4); 8.0585 (0.8); 8.0565 (0.9); 8.0538 (1.0); 8.0518 (0.8); 8.0463 (0.9);
8.0443 (0.9); 8.0417 (0.9); 8.0397 (0.8); 7.8172 (0.7); 7.8110 (0.6); 7.7980 (0.9); 7.7962 (0.9); 7.7918 (0.9); 7.7900 (0.8); 7.7771
(0.7); 7.7709 (0.7); 7.7492 (0.6); 7.7444 (0.6); 7.7309 (0.8); 7.7285 (1.1); 7.7262 (0.8); 7.7238 (1.0); 7.7103 (1.1); 7.7055 (1.0);
7.6507 (1.2); 7.6484 (1.9); 7.6461 (1.1); 7.6300 (0.8); 7.6278 (1.2); 7.6254 (0.6); 7.2617 (41.6); 7.0732 (1.0); 7.0705 (1.0); 7.0610
(1.2); 7.0583 (1.2); 7.0549 (1.2); 7.0522 (1.2); 7.0428 (1.2); 7.0401 (1.1); 6.9728 (1.0); 6.9714 (0.9); 6.9654 (1.0); 6.9518 (0.9);
6.9504 (0.9); 6.9444 (0.9); 5.3003 (2.0); 4.8918 (7.5); 4.1783 (3.4); 4.1656 (3.4); 3.7969 (16.0); 1.5643 (1.7); 1.5504 (0.8); 1.5424
(0.6); 1.5293 (1.2); 1.5161 (0.6); 1.5082 (0.7); 0.8240 (0.6); 0.8132 (1.6); 0.8079 (1.7); 0.8029 (0.8); 0.7975 (1.0); 0.7918 (1.6);
0.7867 (1.4); 0.7765 (0.8); 0.6697 (0.9); 0.6593 (1.7); 0.6560 (1.7); 0.6542 (1.6); 0.6463 (1.5); 0.6422 (1.5); 0.6301 (0.6); 0.0080
(0.7); −0.0002 (26.4); −0.0085 (0.7)
I-71: ¹H-NMR(400.6 MHz, CDCl3):
δ = 8.2101 (1.1); 8.2041 (1.2); 8.0361 (0.6); 8.0343 (0.7); 8.0315 (0.7); 8.0296 (0.7); 8.0240 (0.7); 8.0221 (0.7); 8.0194 (0.7);
8.0175 (0.6); 7.8217 (0.5); 7.8156 (0.5); 7.8024 (0.7); 7.8009 (0.7); 7.7962 (0.7); 7.7947 (0.7); 7.7815 (0.6); 7.7754 (0.5); 7.7207
(0.5); 7.7160 (0.5); 7.7023 (0.6); 7.7000 (0.8); 7.6977 (0.7); 7.6953 (0.8); 7.6817 (0.8); 7.6770 (0.7); 7.5932 (1.5); 7.5911 (0.9);
7.5726 (1.1); 7.5704 (0.6); 7.2613 (42.9); 7.2574 (0.7); 7.0382 (0.7); 7.0356 (0.7); 7.0260 (0.7); 7.0235 (0.7); 7.0199 (0.7); 7.0173
(0.6); 7.0078 (0.6); 7.0052 (0.6); 6.9609 (0.8); 6.9548 (0.8); 6.9411 (0.7); 6.9399 (0.7); 6.9338 (0.7); 6.9325 (0.7); 5.3003 (0.8);
4.8895 (7.8); 4.5046 (2.0); 4.4889 (4.0); 4.4732 (2.0); 3.6905 (16.0); 2.7120 (1.9); 2.6963 (3.8); 2.6806 (1.8); 1.5684 (0.6); 1.4835
(0.6); 1.4739 (0.5); 1.4703 (0.9); 1.4492 (0.6); 0.7779 (1.2); 0.7728 (1.2); 0.7654 (2.1); 0.7593 (1.2); 0.7518 (1.0); 0.7489 (0.5);
0.7397 (1.3); 0.7322 (1.2); 0.7278 (0.8); 0.7221 (0.6); 0.7191 (1.1); 0.7119 (1.3); 0.7063 (1.0); 0.0080 (0.7); −0.0002
(26.5); −0.0085 (0.8)
I-72: ¹H-NMR(400.6 MHz, d₆-DMSO):
δ = 12.9557 (0.7); 8.2209 (3.2); 8.2147 (3.3); 8.0566 (2.0); 8.0545 (2.2); 8.0519 (2.6); 8.0498 (2.3); 8.0445 (2.2); 8.0424 (2.5);
8.0397 (2.5); 8.0377 (2.4); 7.9728 (1.5); 7.9665 (1.4); 7.9519 (2.2); 7.9457 (2.1); 7.9319 (1.6); 7.9257 (1.5); 7.9206 (2.0); 7.9158
(1.9); 7.9021 (2.2); 7.8999 (2.4); 7.8973 (2.2); 7.8952 (2.4); 7.8815 (2.4); 7.8767 (2.3); 7.6173 (2.4); 7.6150 (4.5); 7.6128 (2.7);
7.5967 (2.2); 7.5944 (4.1); 7.5921 (2.3); 7.2479 (2.1); 7.2422 (2.1); 7.2268 (2.0); 7.2211 (2.0); 7.2198 (2.0); 7.2013 (2.3); 7.1988
(2.4); 7.1891 (2.1); 7.1867 (2.4); 7.1829 (2.3); 7.1804 (2.1); 7.1707 (2.3); 7.1682 (2.2); 4.8305 (16.0); 4.0555 (1.1); 4.0377 (3.3);
4.0199 (3.4); 4.0022 (1.1); 3.3194 (16.8); 2.6746 (0.7); 2.6700 (1.0); 2.6653 (0.7); 2.5282 (0.8); 2.5238 (3.2); 2.5191 (4.1); 2.5103
(54.2); 2.5058 (121.3); 2.5012 (172.8); 2.4966 (121.6); 2.4920 (54.2); 2.3329 (0.7); 2.3283 (1.0); 2.3236 (0.7); 1.9886 (16.0);
1.9084 (3.2); 1.5421 (0.6); 1.5288 (1.4); 1.5212 (1.4); 1.5160 (0.9); 1.5080 (2.4); 1.5045 (1.2); 1.4945 (1.4); 1.4871 (1.5); 1.4738
(0.7); 1.3551 (1.3); 1.1922 (4.7); 1.1744 (9.9); 1.1566 (4.7); 0.7032 (1.2); 0.6997 (0.6); 0.6907 (2.5); 0.6855 (3.8); 0.6825 (2.0);
0.6778 (2.7); 0.6725 (1.4); 0.6697 (2.7); 0.6640 (3.7); 0.6570 (2.8); 0.6463 (1.1); 0.6409 (2.5); 0.6333 (3.7); 0.6277 (5.6); 0.6205
(3.9); 0.6152 (3.6); 0.6023 (1.2); 0.0080 (3.5); 0.0064 (0.9); 0.0055 (1.0); 0.0047 (1.2); 0.0039 (1.6); 0.0022 (4.6); −0.0002
(119.2); −0.0026 (6.1); −0.0051 (1.7); −0.0059 (1.3); −0.0067 (1.0); −0.0085 (3.4)
I-73: ¹H-NMR(400.6 MHz, CDCl3):
δ = 8.4849 (1.8); 8.4780 (1.8); 8.3735 (0.9); 8.3696 (1.8); 8.3661 (0.9); 8.0390 (0.7); 8.0370 (0.8); 8.0343 (0.8); 8.0322 (0.8);
8.0269 (0.7); 8.0248 (0.8); 8.0222 (0.8); 8.0201 (0.8); 7.7893 (0.6); 7.7846 (0.6); 7.7709 (0.7); 7.7686 (0.9); 7.7662 (0.7); 7.7639
(0.9); 7.7502 (1.0); 7.7455 (0.9); 7.6727 (0.9); 7.6703 (1.7); 7.6680 (1.0); 7.6520 (0.7); 7.6497 (1.2); 7.6473 (0.7); 7.5231 (0.7);
7.5188 (0.8); 7.5162 (0.8); 7.5119 (0.7); 7.5006 (0.8); 7.4962 (0.8); 7.4936 (0.7); 7.4893 (0.7); 7.2617 (11.0); 7.1094 (0.8); 7.1067
(0.8); 7.0972 (0.8); 7.0946 (0.8); 7.0911 (0.8); 7.0884 (0.8); 7.0789 (0.8); 7.0763 (0.8); 5.2354 (1.6); 5.2180 (1.7); 4.4916 (0.9);
4.4840 (1.0); 4.4758 (2.0); 4.4685 (2.0); 4.4602 (1.0); 4.4529 (1.0); 3.6582 (16.0); 2.6896 (1.2); 2.6732 (2.0); 2.6577 (1.1); 1.7083
(5.8); 1.6908 (5.8); 1.5737 (0.8); 0.0023 (0.5); −0.0002 (14.6); −0.0027 (0.6)
I-74: ¹H-NMR(400.6 MHz, CDCl3):
δ = 8.4898 (1.6); 8.4829 (1.7); 8.3764 (0.9); 8.3725 (1.6); 8.3688 (0.9); 8.0743 (0.6); 8.0722 (0.7); 8.0696 (0.8); 8.0675 (0.7);
8.0621 (0.7); 8.0601 (0.8); 8.0575 (0.8); 8.0554 (0.7); 7.7962 (0.5); 7.7915 (0.5); 7.7778 (0.6); 7.7756 (0.8); 7.7731 (0.7); 7.7709
(0.8); 7.7572 (0.8); 7.7525 (0.8); 7.6721 (0.8); 7.6698 (1.6); 7.6675 (1.0); 7.6515 (0.6); 7.6492 (1.1); 7.6468 (0.6); 7.5349 (0.6);
7.5306 (0.6); 7.5280 (0.7); 7.5237 (0.6); 7.5124 (0.6); 7.5081 (0.6); 7.5055 (0.7); 7.5012 (0.6); 7.2611 (42.0); 7.1333 (0.7); 7.1308
(0.7); 7.1212 (0.7); 7.1186 (0.7); 7.1150 (0.8); 7.1124 (0.7); 7.1028 (0.7); 7.1002 (0.7); 4.9554 (7.2); 4.4230 (0.6); 4.4050 (0.6);
4.3959 (1.3); 4.3779 (1.4); 4.3527 (1.3); 4.3386 (1.4); 4.3256 (0.6); 4.3115 (0.6); 3.7263 (2.8); 3.7131 (0.6); 3.6746 (16.0); 2.8456
(0.7); 2.8315 (0.7); 1.2065 (6.6); 1.1981 (1.5); 1.1886 (6.5); 1.1800 (1.5); 0.0080 (0.6); −0.0002 (26.9); −0.0085 (0.8)
I-75: ¹H-NMR(400.6 MHz, CDCl3):
δ = 8.4881 (1.8); 8.4812 (1.8); 8.3750 (1.0); 8.3712 (1.8); 8.3674 (0.9); 8.0755 (0.7); 8.0734 (0.8); 8.0708 (0.8); 8.0687 (0.8);
8.0634 (0.8); 8.0613 (0.8); 8.0587 (0.8); 8.0566 (0.8); 7.7999 (0.6); 7.7952 (0.6); 7.7815 (0.7); 7.7793 (0.9); 7.7768 (0.7); 7.7746
(0.9); 7.7609 (0.9); 7.7561 (0.9); 7.6748 (1.0); 7.6725 (1.7); 7.6702 (1.0); 7.6542 (0.7); 7.6519 (1.2); 7.6495 (0.7); 7.5276 (0.7);
7.5233 (0.8); 7.5207 (0.8); 7.5164 (0.7); 7.5051 (0.7); 7.5008 (0.8); 7.4982 (0.7); 7.4938 (0.7); 7.2616 (12.7); 7.1347 (0.8); 7.1320
(0.8); 7.1225 (0.8); 7.1199 (0.8); 7.1163 (0.8); 7.1137 (0.8); 7.1041 (0.8); 7.1015 (0.7); 5.3003 (2.8); 4.9522 (8.2); 4.5262 (2.0);
4.5105 (4.1); 4.4949 (2.0); 3.7263 (0.8); 3.6861 (16.0); 2.7202 (1.9); 2.7046 (3.8); 2.6889 (1.9); 1.5751 (1.4); 0.0080 (0.6); −0.0002
(18.9); −0.0085 (0.6)
I-76: ¹H-NMR(400.6 MHz, CDCl3):
δ = 8.4909 (1.2); 8.4841 (1.3); 8.3698 (0.8); 8.3661 (1.3); 8.3626 (0.8); 8.0628 (0.6); 8.0608 (0.7); 8.0581 (0.8); 8.0561 (0.7);
8.0507 (0.7); 8.0486 (0.8); 8.0460 (0.8); 8.0439 (0.7); 7.7910 (0.5); 7.7863 (0.5); 7.7726 (0.6); 7.7703 (0.8); 7.7679 (0.6); 7.7656
(0.8); 7.7520 (0.8); 7.7473 (0.8); 7.6730 (0.8); 7.6707 (1.5); 7.6684 (0.9); 7.6524 (0.6); 7.6501 (1.0); 7.6477 (0.6); 7.5277 (0.6);
7.5234 (0.6); 7.5209 (0.7); 7.5165 (0.6); 7.5053 (0.6); 7.5009 (0.7); 7.4983 (0.6); 7.4940 (0.6); 7.2625 (15.4); 7.1263 (0.7); 7.1237
(0.7); 7.1141 (0.7); 7.1115 (0.7); 7.1079 (0.7); 7.1053 (0.7); 7.0958 (0.7); 7.0932 (0.6); 4.9541 (6.9); 4.4218 (0.6); 4.4039 (0.6);
4.3947 (1.3); 4.3768 (1.3); 4.3508 (1.3); 4.3367 (1.3); 4.3237 (0.6); 4.3096 (0.6); 3.6748 (16.0); 2.8441 (0.6); 2.8300 (0.6); 1.2054
(6.3); 1.1875 (6.2); −0.0002 (9.2)
I-77: ¹H-NMR(400.6 MHz, CDCl3):
δ = 8.4907 (1.0); 8.4840 (1.0); 8.3677 (1.2); 8.0635 (0.7); 8.0614 (0.8); 8.0588 (0.8); 8.0567 (0.8); 8.0514 (0.7); 8.0493 (0.8);
8.0467 (0.8); 8.0446 (0.7); 7.7950 (0.6); 7.7903 (0.6); 7.7766 (0.7); 7.7744 (0.9); 7.7719 (0.7); 7.7697 (0.9); 7.7560 (0.9); 7.7512
(0.9); 7.6765 (0.9); 7.6742 (1.7); 7.6719 (1.0); 7.6559 (0.7); 7.6536 (1.2); 7.6512 (0.7); 7.5262 (0.6); 7.5219 (0.7); 7.5195 (0.8);
7.5150 (0.6); 7.5038 (0.7); 7.4995 (0.7); 7.4969 (0.7); 7.4926 (0.6); 7.2614 (33.9); 7.1278 (0.8); 7.1252 (0.8); 7.1157 (0.8); 7.1131
(0.8); 7.1095 (0.8); 7.1068 (0.8); 7.0973 (0.8); 7.0947 (0.7); 4.9507 (8.0); 4.5244 (1.9); 4.5087 (4.0); 4.4930 (2.0); 3.6860 (16.0);
2.7187 (1.9); 2.7031 (3.8); 2.6874 (1.8); 0.0080 (0.6); −0.0002 (21.3); −0.0085 (0.6)
I-78: ¹H-NMR(400.0 MHz, CDCl3):
δ = 8.2065 (2.5); 8.2005 (2.5); 8.0335 (1.5); 8.0305 (1.6); 8.0213 (1.5); 8.0185 (1.5); 7.8191 (1.2); 7.8130 (1.1); 7.7984 (1.6);
7.7923 (1.5); 7.7788 (1.2); 7.7727 (1.1); 7.7079 (1.0); 7.7031 (1.0); 7.6894 (1.3); 7.6871 (1.7); 7.6825 (1.5); 7.6688 (1.5); 7.6641
(1.4); 7.5863 (3.1); 7.5656 (2.1); 7.2612 (36.6); 7.0326 (1.4); 7.0301 (1.4); 7.0205 (1.4); 7.0179 (1.4); 7.0144 (1.4); 7.0119 (1.3);
7.0022 (1.3); 6.9997 (1.2); 6.9599 (1.7); 6.9529 (1.6); 6.9389 (1.6); 6.9316 (1.5); 4.8882 (16.0); 4.3012 (2.1); 4.2834 (6.4); 4.2655
(6.4); 4.2477 (2.1); 1.5788 (0.8); 1.4995 (1.0); 1.4918 (1.1); 1.4787 (1.7); 1.4705 (0.6); 1.4652 (1.1); 1.4575 (1.1); 1.4442 (0.6);
1.3262 (7.4); 1.3084 (14.9); 1.2905 (7.2); 0.8055 (0.8); 0.8022 (0.6); 0.7921 (2.6); 0.7869 (3.0); 0.7792 (3.7); 0.7745 (3.0); 0.7659
(1.7); 0.7560 (0.7); 0.7481 (1.0); 0.7442 (1.9); 0.7356 (2.7); 0.7314 (2.0); 0.7232 (1.9); 0.7152 (2.5); 0.7098 (1.9); 0.7030 (0.7);
0.6969 (0.7); 0.0080 (0.9); −0.0002 (22.0); −0.0085 (0.7)
I-79: ¹H-NMR(400.6 MHz, CDCl3):
δ = 8.5094 (4.7); 8.5025 (4.8); 8.3669 (3.0); 8.3632 (5.2); 8.3596 (3.0); 8.0593 (2.1); 8.0573 (2.3); 8.0546 (2.5); 8.0526 (2.3);
8.0472 (2.3); 8.0451 (2.5); 8.0425 (2.5); 8.0404 (2.3); 7.7393 (1.8); 7.7346 (1.8); 7.7208 (2.1); 7.7186 (2.6); 7.7161 (2.1); 7.7139
(2.6); 7.7002 (2.6); 7.6955 (2.6); 7.6142 (2.8); 7.6119 (4.9); 7.6096 (2.9); 7.5936 (2.1); 7.5913 (3.5); 7.5889 (2.0); 7.5373 (2.0);
7.5330 (2.1); 7.5304 (2.1); 7.5261 (1.9); 7.5150 (2.1); 7.5107 (2.3); 7.5082 (2.0); 7.5039 (1.9); 7.2610 (56.9); 7.1014 (2.3); 7.0988
(2.3); 7.0892 (2.3); 7.0866 (2.4); 7.0831 (2.4); 7.0804 (2.2); 7.0709 (2.2); 7.0682 (2.1); 5.3495 (1.2); 5.3320 (5.1); 5.3145 (5.2);
5.2970 (1.2); 2.7728 (4.6); 2.1031 (0.9); 2.0465 (1.8); 1.7714 (16.0); 1.7652 (1.7); 1.7539 (15.9); 1.7477 (1.6); 1.2773 (0.6);
1.2594 (1.2); 1.2415 (0.6); 1.2214 (1.5); 0.0080 (2.0); −0.0002 (76.0); −0.0067 (0.7); −0.0085 (2.2)
I-80: ¹H-NMR(400.6 MHz, d₆-DMSO):
δ = 12.6748 (1.4); 8.4653 (2.8); 8.4468 (2.9); 8.2494 (4.0); 8.2451 (3.1); 8.2433 (4.2); 8.0978 (2.6); 8.0958 (2.9); 8.0932 (3.4);
8.0911 (3.0); 8.0857 (2.8); 8.0837 (3.2); 8.0810 (3.2); 8.0789 (3.1); 8.0221 (1.9); 8.0158 (1.8); 8.0024 (2.4); 8.0009 (2.6); 7.9962
(2.4); 7.9947 (2.6); 7.9813 (2.1); 7.9747 (4.1); 7.9699 (2.4); 7.9561 (2.8); 7.9539 (3.2); 7.9514 (2.7); 7.9492 (3.1); 7.9355 (3.2);
7.9307 (3.0); 7.7671 (3.1); 7.7648 (5.8); 7.7626 (3.4); 7.7464 (2.8); 7.7442 (5.0); 7.7419 (2.8); 7.3041 (2.7); 7.2985 (2.7); 7.2972
(2.5); 7.2832 (5.4); 7.2808 (3.9); 7.2773 (2.8); 7.2759 (2.7); 7.2711 (2.8); 7.2686 (3.0); 7.2648 (3.0); 7.2623 (2.7); 7.2527 (3.0);
7.2501 (2.9); 4.8393 (16.0); 4.3306 (2.6); 4.3123 (4.1); 4.2940 (2.6); 4.2758 (0.5); 3.6216 (0.9); 3.6177 (5.2); 3.6155 (2.9); 3.6115
(2.7); 3.6093 (2.1); 3.6074 (3.6); 3.6028 (3.9); 3.6011 (12.0); 3.5989 (4.3); 3.5950 (3.8); 3.5930 (2.1); 3.5908 (2.7); 3.5867 (2.8);
3.5845 (5.4); 3.5806 (1.0); 3.3212 (11.7); 2.6751 (0.6); 2.6705 (0.8); 2.6658 (0.6); 2.5242 (2.1); 2.5196 (2.8); 2.5108 (40.1);
2.5062 (91.1); 2.5016 (129.2); 2.4970 (90.8); 2.4924 (40.8); 2.3333 (0.5); 2.3287 (0.8); 2.3241 (0.5); 2.1829 (0.8); 1.9087 (0.7);
1.7808 (0.8); 1.7761 (5.3); 1.7685 (4.2); 1.7640 (2.6); 1.7595 (15.2); 1.7548 (2.7); 1.7505 (4.2); 1.7429 (5.2); 1.7384 (0.8); 1.3554
(7.3); 1.3262 (15.9); 1.3080 (15.8); 0.1457 (0.6); 0.0079 (6.1); 0.0062 (1.3); 0.0054 (1.5); 0.0045 (1.9); −0.0002 (240.5); −0.0028
(10.9); −0.0052 (2.8); −0.0061 (2.2); −0.0069 (1.9); −0.0085 (6.9); −0.0108 (0.9); −0.0117 (0.8); −0.0124 (0.8); −0.0132 (0.6); −0.0140
(0.6); −0.1495 (0.7)
I-81: ¹H-NMR(400.6 MHz, CDCl3):
δ = 8.5080 (3.0); 8.5010 (3.0); 8.3784 (1.6); 8.3746 (2.7); 8.0840 (1.2); 8.0773 (1.4); 8.0719 (1.3); 8.0698 (1.3); 8.0673 (1.4);
7.7923 (1.1); 7.7875 (1.1); 7.7736 (1.2); 7.7716 (1.4); 7.7691 (1.1); 7.7668 (1.4); 7.7531 (1.5); 7.7484 (1.6); 7.6572 (2.8); 7.6365
(2.0); 7.5478 (0.8); 7.5433 (0.9); 7.5366 (0.8); 7.5186 (2.3); 7.2858 (0.8); 7.2602 (283.4); 7.2535 (1.7); 7.2326 (1.8); 7.1432 (1.4);
7.1407 (1.4); 7.1311 (1.2); 7.1285 (1.3); 7.1249 (1.2); 7.1223 (1.2); 7.1127 (1.3); 7.1101 (1.2); 6.9965 (1.6); 5.0311 (16.0); 4.1136
(0.5); 2.7772 (3.0); 2.1074 (0.9); 2.0459 (2.6); 1.8552 (0.6); 1.4322 (4.0); 1.2842 (0.6); 1.2775 (0.9); 1.2598 (1.9); 1.2435 (2.5);
1.2227 (1.5); 0.1459 (1.3); 0.0338 (0.5); 0.0255 (1.2); 0.0176 (0.8); 0.0080 (12.8); −0.0002 (462.3); −0.0085 (12.5); −0.0277
(3.2); −0.1492 (1.3)
I-82: ¹H-NMR(400.6 MHz, CDCl3):
δ = 8.5045 (1.9); 8.4976 (1.9); 8.3531 (1.0); 8.3493 (1.8); 8.3455 (1.0); 8.1167 (0.7); 8.1146 (0.8); 8.1121 (0.8); 8.1100 (0.8);
8.1046 (0.7); 8.1024 (0.8); 8.1001 (0.8); 8.0979 (0.8); 7.8481 (0.5); 7.8455 (1.0); 7.8437 (0.6); 7.8409 (1.0); 7.8276 (1.1); 7.8230
(1.1); 7.8043 (1.0); 7.8015 (1.7); 7.7991 (1.2); 7.7838 (0.6); 7.7813 (0.7); 7.5112 (0.7); 7.5068 (0.8); 7.5043 (0.8); 7.4999 (0.7);
7.4888 (0.7); 7.4845 (0.8); 7.4820 (0.8); 7.4776 (0.7); 7.2645 (5.7); 7.2098 (0.8); 7.2067 (0.8); 7.1977 (0.8); 7.1945 (0.8); 7.1919
(0.8); 7.1888 (0.8); 7.1798 (0.8); 7.1766 (0.7); 5.3006 (3.1); 3.8261 (7.5); 3.5681 (0.9); 3.5552 (16.0); 3.5380 (2.0); 3.5226 (0.9);
2.5160 (1.6); 2.5007 (2.4); 2.4860 (1.6); −0.0002 (8.4)
I-83: ¹H-NMR(400.6 MHz, CDCl3):
δ = 8.0422 (0.6); 8.0403 (0.6); 8.0377 (0.7); 8.0357 (0.6); 8.0301 (0.6); 8.0282 (0.7); 8.0256 (0.7); 8.0236 (0.6); 7.7573 (0.6);
7.7551 (0.7); 7.7526 (0.6); 7.7504 (0.7); 7.7367 (0.7); 7.7320 (0.6); 7.6417 (0.8); 7.6396 (1.3); 7.6374 (0.8); 7.6211 (0.6); 7.6190
(1.0); 7.6168 (0.6); 7.2612 (30.9); 7.1092 (0.6); 7.1068 (0.6); 7.0971 (0.6); 7.0946 (0.6); 7.0909 (0.6); 7.0884 (0.6); 7.0788 (0.6);
7.0762 (0.6); 5.3001 (0.8); 5.2732 (1.3); 5.2558 (1.3); 3.8017 (16.0); 1.7227 (5.8); 1.7052 (5.7); 0.0080 (0.5); −0.0002
(19.0); −0.0085 (0.6)
I-84: ¹H-NMR(400.6 MHz, CDCl3):
δ = 9.0998 (1.0); 8.7062 (0.8); 8.6990 (0.8); 8.5164 (3.2); 8.5094 (3.1); 8.4023 (1.8); 8.3986 (2.9); 8.3949 (1.7); 8.0893 (1.3);
8.0868 (1.5); 8.0795 (1.2); 8.0772 (1.3); 8.0747 (1.3); 8.0725 (1.3); 8.0313 (0.6); 7.8007 (1.0); 7.7960 (1.1); 7.7801 (1.5); 7.7754
(1.6); 7.7617 (1.6); 7.7570 (1.6); 7.6650 (1.6); 7.6627 (2.7); 7.6603 (1.5); 7.6444 (1.3); 7.6420 (2.1); 7.6397 (1.3); 7.5832 (0.9);
7.5790 (1.0); 7.5721 (0.9); 7.5611 (0.8); 7.5543 (1.0); 7.5498 (0.9); 7.5186 (1.6); 7.2874 (1.4); 7.2689 (0.8); 7.2602 (301.0);
7.2528 (1.7); 7.2432 (0.6); 7.2265 (0.5); 7.1514 (1.3); 7.1488 (1.4); 7.1392 (1.4); 7.1366 (1.5); 7.1329 (1.4); 7.1304 (1.4); 7.1208
(1.3); 7.1182 (1.2); 6.9966 (1.6); 6.9797 (0.5); 5.0344 (16.0); 4.2590 (1.1); 4.1139 (0.5); 2.7781 (8.9); 2.2718 (1.2); 2.1117 (2.5);
2.0462 (2.5); 1.4322 (11.1); 1.3327 (0.6); 1.2841 (1.0); 1.2777 (1.0); 1.2599 (2.5); 1.2549 (2.2); 1.2433 (1.6); 1.2227 (3.1); 0.1458
(1.3); 0.0270 (2.4); 0.0080 (12.5); 0.0042 (4.7); −0.0002 (480.0); −0.0085 (12.5); −0.0174 (0.6); −0.0225 (0.6); −0.0338 (0.6); −0.1495 (1.4)
I-85: ¹H-NMR(400.6 MHz, CDCl3):
δ = 8.4876 (3.9); 8.4808 (3.8); 8.3693 (2.8); 8.3659 (4.0); 8.0736 (1.9); 8.0709 (2.0); 8.0615 (2.0); 8.0588 (1.9); 7.7872 (1.1);
7.7826 (1.1); 7.7666 (2.0); 7.7641 (1.8); 7.7621 (1.7); 7.7482 (1.6); 7.7435 (1.5); 7.6570 (3.4); 7.6364 (2.4); 7.5245 (1.4); 7.5202
(1.7); 7.5179 (1.6); 7.5135 (1.3); 7.5020 (1.4); 7.4977 (1.6); 7.4953 (1.5); 7.4910 (1.2); 7.2622 (8.4); 7.1301 (1.7); 7.1279 (1.5);
7.1180 (1.8); 7.1156 (1.8); 7.1119 (1.8); 7.1095 (1.5); 7.0997 (1.5); 7.0974 (1.3); 4.9503 (16.0); 4.3266 (2.1); 4.3088 (6.2); 4.2910
(6.2); 4.2732 (2.1); 2.7720 (1.6); 2.0452 (0.7); 1.3351 (7.0); 1.3173 (13.9); 1.2995 (6.8); 1.2594 (0.5); −0.0002 (13.1); −0.0084 (0.6)
I-86: ¹H-NMR(400.6 MHz, CDCl3):
δ = 8.4901 (3.1); 8.4832 (3.1); 8.3650 (1.7); 8.3612 (3.1); 8.3573 (1.7); 8.0637 (1.2); 8.0617 (1.4); 8.0590 (1.5); 8.0569 (1.4);
8.0516 (1.3); 8.0495 (1.4); 8.0469 (1.5); 8.0448 (1.4); 7.7819 (1.1); 7.7772 (1.1); 7.7635 (1.2); 7.7613 (1.7); 7.7588 (1.3); 7.7565
(1.6); 7.7428 (1.6); 7.7381 (1.6); 7.6604 (1.7); 7.6580 (3.1); 7.6557 (1.9); 7.6398 (1.3); 7.6374 (2.2); 7.6351 (1.3); 7.5229 (1.3);
7.5186 (1.3); 7.5161 (1.4); 7.5117 (1.3); 7.5005 (1.3); 7.4962 (1.4); 7.4936 (1.3); 7.4892 (1.2); 7.2618 (13.7); 7.1236 (1.4); 7.1210
(1.5); 7.1114 (1.4); 7.1088 (1.5); 7.1052 (1.4); 7.1026 (1.4); 7.0931 (1.4); 7.0904 (1.4); 5.3001 (4.4); 4.9488 (15.3); 4.3246 (1.9);
4.3068 (6.1); 4.2890 (6.1); 4.2712 (2.0); 2.7729 (1.6); 2.0453 (1.2); 1.3342 (7.8); 1.3164 (16.0); 1.2986 (7.7); 1.2595 (0.8); 0.0080
(0.6); −0.0002 (21.4); −0.0085 (0.6)
I-87: ¹H-NMR(400.6 MHz, CDCl3):
δ = 8.2225 (1.1); 8.2205 (0.8); 8.2183 (0.8); 8.2163 (1.1); 8.0975 (0.7); 8.0955 (0.7); 8.0928 (0.8); 8.0908 (0.7); 8.0854 (0.7);
8.0833 (0.8); 8.0807 (0.8); 8.0786 (0.7); 7.8710 (0.6); 7.8648 (0.6); 7.8522 (0.7); 7.8499 (0.7); 7.8460 (0.7); 7.8437 (0.7); 7.8311
(0.6); 7.8249 (0.6); 7.8108 (0.6); 7.8060 (0.6); 7.7924 (0.6); 7.7901 (0.9); 7.7877 (0.7); 7.7854 (0.8); 7.7718 (0.9); 7.7671 (0.9);
7.7070 (0.9); 7.7046 (1.6); 7.7023 (1.0); 7.6863 (0.6); 7.6840 (1.1); 7.6816 (0.6); 7.2615 (13.6); 7.1531 (0.8); 7.1504 (0.8); 7.1409
(0.9); 7.1382 (1.0); 7.1348 (1.0); 7.1321 (1.0); 7.1226 (1.0); 7.1199 (0.9); 7.0129 (0.7); 7.0115 (0.8); 7.0055 (0.8); 7.0041 (0.8);
6.9917 (0.7); 6.9903 (0.8); 6.9844 (0.8); 6.9829 (0.7); 5.3003 (0.7); 4.9083 (6.5); 4.7300 (0.7); 4.7118 (1.1); 4.6936 (0.8); 3.7782
(16.0); 1.5624 (1.3); 1.5106 (6.3); 1.4926 (6.2); 0.0079 (0.6); −0.0002 (20.0); −0.0085 (0.6)
I-88: ¹H-NMR(400.6 MHz, CDCl3):
δ = 8.2170 (1.2); 8.2148 (0.9); 8.2127 (1.0); 8.2108 (1.2); 8.0814 (0.7); 8.0795 (0.7); 8.0768 (0.8); 8.0748 (0.7); 8.0693 (0.7);
8.0673 (0.7); 8.0646 (0.8); 8.0627 (0.6); 7.8706 (0.6); 7.8644 (0.6); 7.8517 (0.8); 7.8495 (0.8); 7.8455 (0.8); 7.8433 (0.7); 7.8306
(0.7); 7.8244 (0.6); 7.7887 (0.6); 7.7839 (0.6); 7.7702 (0.7); 7.7680 (0.9); 7.7655 (0.8); 7.7633 (0.9); 7.7496 (0.9); 7.7449 (0.8);
7.6603 (1.0); 7.6581 (1.5); 7.6559 (0.9); 7.6396 (0.7); 7.6375 (1.1); 7.6353 (0.6); 7.2625 (6.7); 7.1273 (0.7); 7.1248 (0.7); 7.1151
(0.7); 7.1126 (0.7); 7.1089 (0.8); 7.1064 (0.7); 7.0968 (0.7); 7.0942 (0.6); 7.0000 (0.8); 6.9987 (0.8); 6.9927 (0.9); 6.9913 (0.8);
6.9789 (0.8); 6.9774 (0.8); 6.9715 (0.8); 6.9701 (0.7); 5.3003 (1.5); 4.9496 (8.3); 4.5246 (2.0); 4.5090 (4.2); 4.4933 (2.1); 3.7260
(1.0); 3.6846 (16.0); 2.7198 (2.0); 2.7041 (4.0); 2.6884 (1.9); 1.5699 (0.6); −0.0002 (9.3)
I-89: ¹H-NMR(400.0 MHz, d₆-DMSO):
δ = 12.2270 (0.6); 8.2508 (1.4); 8.2446 (1.4); 8.1006 (1.2); 8.0959 (1.1); 8.0837 (1.9); 8.0223 (0.5); 8.0161 (0.5); 8.0016 (0.9);
7.9953 (0.9); 7.9876 (0.7); 7.9821 (1.0); 7.9752 (0.6); 7.9670 (0.9); 7.9642 (0.8); 7.9624 (0.9); 7.9483 (0.8); 7.9437 (0.7); 7.7125
(1.6); 7.6919 (1.4); 7.3045 (0.9); 7.2981 (0.9); 7.2829 (1.6); 7.2764 (0.9); 7.2727 (0.8); 7.2704 (0.9); 7.2663 (0.8); 7.2641 (0.8);
7.2542 (0.8); 7.2519 (0.8); 4.7456 (4.4); 4.1694 (0.6); 3.6177 (1.1); 3.6114 (0.7); 3.6074 (1.0); 3.6011 (2.8); 3.5951 (1.0); 3.5910
(0.7); 3.5846 (1.2); 3.3213 (16.0); 2.5243 (0.5); 2.5195 (0.7); 2.5108 (11.1); 2.5063 (24.1); 2.5017 (33.8); 2.4972 (24.0); 2.4927
(10.8); 2.4756 (1.1); 2.4604 (1.1); 2.3774 (1.1); 2.3589 (1.1); 2.3383 (0.7); 2.3199 (0.7); 1.7761 (1.2); 1.7682 (1.2); 1.7595 (3.5);
1.7508 (1.2); 1.7431 (1.2); 1.3556 (3.0); 1.2348 (0.6); 1.1383 (4.7); 1.1217 (4.6); −0.0002 (17.2); −0.0085 (0.5)
I-90: ¹H-NMR(400.6 MHz, CDCl3):
δ = 8.2013 (2.4); 8.1993 (1.9); 8.1952 (2.5); 8.0821 (1.5); 8.0801 (1.6); 8.0774 (1.7); 8.0755 (1.5); 8.0700 (1.6); 8.0680 (1.7);
8.0653 (1.8); 8.0634 (1.5); 7.8604 (1.2); 7.8542 (1.2); 7.8414 (1.5); 7.8393 (1.5); 7.8352 (1.5); 7.8331 (1.4); 7.8204 (1.3); 7.8142
(1.3); 7.7761 (1.2); 7.7714 (1.2); 7.7576 (1.6); 7.7555 (1.8); 7.7528 (1.6); 7.7508 (1.7); 7.7371 (1.7); 7.7323 (1.7); 7.6254 (2.1);
7.6234 (3.4); 7.6212 (1.9); 7.6049 (1.6); 7.6028 (2.5); 7.6005 (1.4); 7.2614 (20.9); 7.1265 (1.6); 7.1240 (1.5); 7.1143 (1.6); 7.1119
(1.6); 7.1081 (1.6); 7.1056 (1.5); 7.0959 (1.6); 7.0934 (1.4); 6.9999 (1.7); 6.9985 (1.7); 6.9926 (1.8); 6.9913 (1.6); 6.9788 (1.7);
6.9774 (1.6); 6.9714 (1.7); 5.3002 (2.4); 4.9648 (16.0); 4.2712 (1.4); 4.2552 (1.5); 4.2442 (2.2); 4.2282 (2.2); 4.1520 (2.3); 4.1322
(2.4); 4.1250 (1.6); 4.1052 (1.6); 3.8525 (0.7); 3.8387 (0.8); 3.8317 (1.5); 3.8179 (1.6); 3.8110 (2.9); 3.7974 (1.2); 3.7931 (1.9);
3.7888 (2.1); 3.7710 (2.0); 3.7453 (1.2); 3.7270 (1.8); 3.7086 (1.5); 3.6875 (0.9); 3.5632 (1.9); 3.5495 (2.0); 3.5410 (1.7); 3.5273
(1.7); 2.6265 (0.6); 2.6094 (0.8); 2.5922 (0.7); 2.0324 (0.7); 2.0200 (0.9); 2.0116 (0.5); 2.0070 (0.6); 1.9991 (0.9); 1.9872 (0.7);
1.9803 (0.6); 1.9666 (0.5); 1.6600 (0.5); 1.6426 (0.8); 1.6280 (1.0); 1.6085 (1.0); 1.5937 (0.7); 1.5767 (0.5); 1.5579 (5.7); 1.2556
(0.9); 0.0080 (1.0); −0.0002 (32.8); −0.0057 (0.7); −0.0065 (0.6); −0.0086 (1.1)
I-91: ¹H-NMR(400.6 MHz, CDCl3):
δ = 8.4841 (3.1); 8.4772 (3.2); 8.3609 (1.6); 8.3571 (3.1); 8.3536 (1.7); 8.0380 (1.2); 8.0360 (1.4); 8.0334 (1.5); 8.0313 (1.4);
8.0259 (1.3); 8.0238 (1.4); 8.0212 (1.5); 8.0191 (1.4); 7.7681 (1.1); 7.7633 (1.1); 7.7497 (1.2); 7.7474 (1.6); 7.7450 (1.3); 7.7427
(1.6); 7.7291 (1.6); 7.7243 (1.6); 7.6514 (1.7); 7.6491 (3.0); 7.6467 (1.9); 7.6308 (1.3); 7.6284 (2.1); 7.6261 (1.2); 7.5152 (1.3);
7.5109 (1.4); 7.5083 (1.4); 7.5040 (1.3); 7.4927 (1.3); 7.4884 (1.4); 7.4857 (1.4); 7.4814 (1.3); 7.2623 (10.0); 7.1034 (1.4); 7.1008
(1.5); 7.0913 (1.4); 7.0886 (1.4); 7.0851 (1.4); 7.0824 (1.4); 7.0729 (1.4); 7.0703 (1.4); 5.2574 (0.8); 5.2400 (2.9); 5.2226 (3.0);
5.2052 (0.8); 4.2945 (0.9); 4.2881 (1.0); 4.2858 (0.5); 4.2766 (3.0); 4.2704 (3.0); 4.2588 (3.1); 4.2526 (3.0); 4.2410 (1.0); 4.2349
(1.0); 1.7179 (11.1); 1.7005 (11.0); 1.3054 (7.5); 1.2876 (16.0); 1.2698 (7.4); −0.0002 (15.1); −0.0085 (0.5)
I-92: ¹H-NMR(400.6 MHz, CDCl3):
δ = 8.2153 (1.1); 8.2133 (0.9); 8.2112 (0.9); 8.2091 (1.2); 8.2073 (0.8); 8.0826 (0.6); 8.0805 (0.7); 8.0779 (0.8); 8.0758 (0.7);
8.0704 (0.7); 8.0684 (0.8); 8.0658 (0.8); 8.0637 (0.8); 7.8637 (0.6); 7.8575 (0.6); 7.8449 (0.7); 7.8426 (0.8); 7.8387 (0.8); 7.8364
(0.8); 7.8238 (0.7); 7.8176 (0.7); 7.8057 (0.6); 7.8010 (0.6); 7.7874 (0.7); 7.7851 (1.0); 7.7827 (0.7); 7.7804 (1.0); 7.7668 (1.0);
7.7621 (1.0); 7.7060 (0.9); 7.7036 (1.6); 7.7013 (1.0); 7.6854 (0.6); 7.6830 (1.0); 7.6807 (0.6); 7.2631 (7.7); 7.1452 (0.7); 7.1426
(0.8); 7.1331 (0.7); 7.1304 (0.8); 7.1269 (0.8); 7.1243 (0.8); 7.1148 (0.8); 7.1121 (0.9); 7.0094 (0.8); 7.0078 (0.8); 7.0020 (0.8);
7.0004 (0.9); 6.9882 (0.8); 6.9867 (0.8); 6.9808 (0.8); 6.9793 (0.8); 5.3006 (1.3); 4.8618 (8.2); 3.6806 (16.0); 2.6050 (3.3); 2.5917
(3.2); 1.5888 (1.5); 1.3186 (5.8); 1.3017 (5.8); −0.0002 (11.0)
I-93: ¹H-NMR(400.6 MHz, CDCl3):
δ = 8.1976 (2.5); 8.1914 (2.6); 8.0650 (1.5); 8.0631 (1.6); 8.0603 (1.8); 8.0584 (1.6); 8.0528 (1.7); 8.0509 (1.8); 8.0482 (1.8);
8.0464 (1.6); 7.8583 (1.3); 7.8522 (1.3); 7.8393 (1.5); 7.8373 (1.6); 7.8331 (1.6); 7.8311 (1.6); 7.8183 (1.4); 7.8121 (1.3); 7.7691
(1.2); 7.7644 (1.2); 7.7507 (1.4); 7.7484 (1.9); 7.7460 (1.5); 7.7438 (1.8); 7.7302 (1.9); 7.7254 (1.9); 7.6649 (2.1); 7.6627 (3.6);
7.6604 (2.1); 7.6442 (1.5); 7.6421 (2.4); 7.6398 (1.4); 7.2609 (37.0); 7.1145 (1.6); 7.1119 (1.6); 7.1024 (1.6); 7.0997 (1.6); 7.0962
(1.7); 7.0936 (1.5); 7.0841 (1.5); 7.0814 (1.5); 6.9975 (2.0); 6.9902 (1.9); 6.9765 (1.8); 6.9691 (1.8); 5.3002 (6.6); 5.0017 (16.0);
4.3332 (0.5); 4.3176 (2.3); 4.2984 (3.3); 4.2919 (0.7); 4.2822 (1.2); 4.1967 (1.3); 4.1810 (3.4); 4.1771 (2.0); 4.1618 (5.8); 4.1541
(1.3); 4.1515 (1.2); 4.1453 (1.1); 3.9016 (0.9); 3.8846 (1.6); 3.8807 (1.6); 3.8681 (1.1); 3.8638 (2.6); 3.8473 (1.3); 3.8124 (1.2);
3.7947 (1.7); 3.7788 (1.4); 3.7743 (1.2); 3.7581 (0.8); 2.0003 (0.7); 1.9877 (0.7); 1.9796 (0.9); 1.9653 (0.8); 1.9499 (0.7); 1.9350
(0.7); 1.9237 (0.7); 1.9196 (1.0); 1.9149 (0.9); 1.9071 (2.0); 1.9026 (1.3); 1.8990 (1.2); 1.8903 (1.9); 1.8863 (1.6); 1.8726 (0.9);
1.8691 (1.3); 1.6458 (0.9); 1.6413 (0.5); 1.6322 (0.7); 1.6241 (0.8); 1.6152 (0.8); 1.5974 (0.7); 1.5487 (8.9); 0.0079 (1.4); −0.0002
(50.3); −0.0085 (1.9)
I-94: ¹H-NMR(400.0 MHz, d₆-DMSO):
δ = 8.2765 (1.2); 8.2708 (1.3); 8.1417 (0.8); 8.1390 (0.9); 8.1295 (0.9); 8.1268 (0.9); 8.0737 (0.8); 8.0677 (0.7); 8.0145 (0.6);
8.0098 (0.6); 7.9942 (0.9); 7.9896 (0.9); 7.9753 (0.8); 7.9707 (0.7); 7.6634 (1.6); 7.6429 (1.4); 7.3508 (0.8); 7.3485 (0.8); 7.3386
(0.7); 7.3364 (0.8); 7.3321 (0.8); 7.3298 (0.7); 7.3201 (0.8); 7.3177 (0.7); 7.2963 (0.8); 7.2899 (0.9); 7.2750 (0.8); 7.2684 (0.8);
5.1911 (1.5); 5.1736 (1.5); 3.3189 (16.0); 2.5238 (0.7); 2.5191 (1.0); 2.5103 (18.0); 2.5057 (40.1); 2.5012 (56.5); 2.4966 (40.2);
2.4921 (18.2); 1.9884 (1.1); 1.5950 (4.6); 1.5776 (4.6); 1.3554 (0.8); 1.2356 (0.8); 1.1745 (0.6); 0.0080 (1.3); −0.0002
(50.2); −0.0085 (1.5)
I-95: ¹H-NMR(400.6 MHz, CDCl3):
δ = 8.2023 (1.1); 8.1961 (1.2); 8.0763 (0.6); 8.0744 (0.7); 8.0717 (0.7); 8.0698 (0.7); 8.0642 (0.7); 8.0623 (0.7); 8.0595 (0.7);
8.0577 (0.7); 7.8609 (0.6); 7.8547 (0.5); 7.8419 (0.6); 7.8398 (0.7); 7.8357 (0.7); 7.8336 (0.7); 7.8209 (0.6); 7.8147 (0.6); 7.7755
(0.5); 7.7707 (0.5); 7.7570 (0.6); 7.7548 (0.8); 7.7523 (0.7); 7.7501 (0.8); 7.7365 (0.8); 7.7317 (0.8); 7.6538 (0.9); 7.6517 (1.5);
7.6496 (0.9); 7.6332 (0.6); 7.6311 (1.1); 7.6289 (0.6); 7.2612 (14.2); 7.1232 (0.7); 7.1206 (0.7); 7.1110 (0.6); 7.1085 (0.7); 7.1048
(0.7); 7.1023 (0.7); 7.0927 (0.6); 7.0901 (0.6); 6.9979 (0.8); 6.9917 (0.8); 6.9779 (0.7); 6.9706 (0.8); 4.9783 (7.7); 4.4228 (2.0);
4.4059 (4.1); 4.3890 (2.1); 2.7825 (2.2); 2.7657 (4.4); 2.7487 (2.1); 2.1441 (16.0); 1.5776 (0.9); −0.0002 (17.6); −0.0085 (0.6)
I-96: ¹H-NMR(400.6 MHz, CDCl3):
δ = 8.2002 (1.1); 8.1941 (1.2); 8.0656 (0.6); 8.0629 (0.7); 8.0535 (0.7); 8.0508 (0.7); 7.8599 (0.5); 7.8537 (0.5); 7.8408 (0.6);
7.8388 (0.7); 7.8347 (0.7); 7.8327 (0.6); 7.8199 (0.6); 7.8137 (0.6); 7.7512 (0.6); 7.7490 (0.8); 7.7466 (0.6); 7.7444 (0.7); 7.7307
(0.7); 7.7260 (0.7); 7.6545 (1.4); 7.6339 (1.0); 7.2613 (9.9); 7.1153 (0.6); 7.1128 (0.6); 7.1031 (0.6); 7.1006 (0.6); 7.0970 (0.7);
7.0945 (0.6); 7.0849 (0.6); 7.0823 (0.6); 6.9969 (0.8); 6.9896 (0.8); 6.9757 (0.7); 6.9684 (0.8); 5.0026 (7.3); 4.3973 (1.8); 4.3893
(1.0); 4.3858 (1.9); 4.3819 (1.0); 4.3741 (2.0); 3.6510 (2.1); 3.6431 (1.0); 3.6393 (2.0); 3.6357 (1.1); 3.6277 (2.0); 3.3793
(16.0); −0.0002 (12.0)
I-97: ¹H-NMR(400.6 MHz, CDCl3):
δ = 8.2122 (1.2); 8.2082 (1.0); 8.2060 (1.3); 8.0905 (0.7); 8.0885 (0.8); 8.0859 (0.9); 8.0838 (0.8); 8.0784 (0.8); 8.0763 (0.9);
8.0737 (0.9); 8.0717 (0.8); 7.8644 (0.7); 7.8582 (0.7); 7.8456 (0.8); 7.8433 (0.8); 7.8394 (0.8); 7.8372 (0.8); 7.8245 (0.8); 7.8183
(0.7); 7.8093 (0.6); 7.8046 (0.6); 7.7910 (0.7); 7.7887 (1.0); 7.7863 (0.8); 7.7840 (1.0); 7.7704 (1.0); 7.7657 (1.0); 7.7121 (1.0);
7.7097 (1.9); 7.7075 (1.2); 7.6915 (0.7); 7.6891 (1.2); 7.6868 (0.7); 7.2617 (15.3); 7.1504 (0.9); 7.1477 (0.9); 7.1382 (0.8); 7.1356
(0.9); 7.1321 (0.9); 7.1294 (0.9); 7.1199 (0.9); 7.1173 (0.8); 7.0091 (0.9); 7.0077 (0.9); 7.0017 (0.9); 7.0003 (0.9); 6.9880 (0.9);
6.9866 (0.9); 6.9806 (0.9); 6.9792 (0.9); 5.3003 (6.8); 4.9460 (7.7); 4.1838 (3.5); 4.1704 (3.5); 3.7928 (16.0); 1.5619 (8.1); 1.2557
(0.5); 0.0080 (0.5); −0.0002 (20.8); −0.0085 (0.8)
I-98: ¹H-NMR(400.0 MHz, d₆-DMSO):
δ = 8.2778 (3.2); 8.2716 (3.4); 8.1260 (1.9); 8.1241 (2.1); 8.1214 (2.4); 8.1195 (2.1); 8.1138 (2.0); 8.1120 (2.3); 8.1093 (2.3);
8.1074 (2.1); 8.0448 (1.4); 8.0386 (1.3); 8.0238 (2.0); 8.0177 (2.0); 8.0040 (1.5); 7.9979 (3.1); 7.9932 (1.6); 7.9792 (2.1); 7.9774
(2.3); 7.9746 (2.0); 7.9727 (2.1); 7.9588 (2.0); 7.9540 (1.9); 7.6868 (4.1); 7.6662 (3.7); 7.3089 (2.2); 7.3020 (4.2); 7.2921 (2.1);
7.2894 (3.4); 7.2862 (3.1); 7.2831 (2.9); 7.2737 (2.0); 7.2713 (1.9); 4.9361 (16.0); 4.0561 (0.5); 4.0382 (1.7); 4.0205 (1.7); 4.0027
(0.6); 3.3224 (3.0); 2.5246 (0.7); 2.5199 (1.0); 2.5112 (17.3); 2.5066 (38.2); 2.5021 (53.5); 2.4975 (37.8); 2.4929 (16.9); 1.9889
(7.4); 1.9094 (3.8); 1.1925 (2.0); 1.1747 (4.0); 1.1569 (2.0); 0.0080 (1.2); −0.0002 (45.3); −0.0085 (1.3)
I-99: ¹H-NMR(400.0 MHz, CDCl3):
δ = 8.1584 (2.0); 8.1520 (2.2); 8.1026 (1.4); 8.1000 (1.4); 8.0878 (1.4); 7.8516 (0.8); 7.8459 (0.7); 7.8316 (1.2); 7.8118 (0.8);
7.8056 (0.8); 7.7909 (1.1); 7.7862 (1.0); 7.7704 (1.5); 7.7659 (1.4); 7.7517 (1.4); 7.7470 (1.3); 7.6111 (2.7); 7.5906 (2.1); 7.2605
(66.6); 7.1759 (1.3); 7.1637 (1.3); 7.1599 (1.2); 7.1453 (1.2); 6.9948 (1.6); 6.9869 (1.5); 6.9789 (1.2); 6.9730 (1.5); 6.9667 (1.4);
5.0311 (13.8); 5.0208 (0.9); 5.0075 (0.6); 4.9719 (0.6); 4.8594 (0.9); 3.8864 (1.1); 3.1157 (0.5); 2.2716 (1.8); 1.4322 (16.0);
1.2540 (2.8); 1.2224 (0.7); 0.8796 (0.5); 0.0690 (0.9); 0.0080 (2.0); −0.0002 (81.2); −0.0085 (2.6)
II-01: ¹H-NMR(400.6 MHz, CDCl3):
δ = 8.2147 (1.1); 8.2104 (1.4); 8.2070 (1.2); 8.2026 (1.2); 8.1992 (1.5); 8.1983 (1.5); 8.1949 (1.2); 8.0912 (2.0); 8.0892 (1.6);
8.0870 (1.6); 8.0849 (2.1); 8.0832 (1.5); 7.8797 (1.0); 7.8751 (1.0); 7.8605 (1.2); 7.8563 (1.6); 7.8524 (1.2); 7.8378 (1.2); 7.8332
(1.1); 7.7939 (1.1); 7.7876 (1.0); 7.7754 (1.2); 7.7727 (1.3); 7.7691 (1.2); 7.7665 (1.3); 7.7542 (1.1); 7.7479 (1.1); 7.3178 (1.2);
7.3150 (1.3); 7.3057 (1.2); 7.3029 (1.3); 7.2986 (1.2); 7.2958 (1.2); 7.2864 (1.2); 7.2836 (1.2); 7.2621 (17.1); 6.9957 (1.4); 6.9942
(1.4); 6.9882 (1.4); 6.9868 (1.4); 6.9745 (1.3); 6.9730 (1.4); 6.9670 (1.4); 6.9655 (1.4); 4.8917 (15.2); 4.3018 (1.9); 4.2840 (6.0);
4.2662 (6.1); 4.2484 (2.0); 2.0455 (0.6); 1.5590 (7.9); 1.5581 (8.4); 1.3141 (7.6); 1.2963 (16.0); 1.2877 (0.5); 1.2784 (7.7); 1.2644
(0.6); 1.2596 (0.7); 0.8819 (0.9); 0.0080 (0.6); −0.0002 (22.3); −0.0085 (0.7)
II-03: ¹H-NMR(599.6 MHz, CDCl3):
δ = 8.2192 (7.4); 8.2115 (7.3); 8.1073 (11.4); 8.1034 (11.3); 7.9010 (3.7); 7.8981 (3.7); 7.8856 (7.1); 7.8730 (3.9); 7.8702 (3.6);
7.7990 (3.6); 7.7949 (3.6); 7.7848 (5.7); 7.7825 (5.5); 7.7725 (3.8); 7.7684 (3.4); 7.3295 (5.8); 7.3212 (6.1); 7.3169 (5.9); 7.3087
(5.3); 7.2638 (14.2); 7.0023 (6.5); 6.9979 (6.5); 6.9881 (6.3); 6.9837 (6.1); 5.9493 (0.3); 5.2999 (12.4); 5.0641 (0.3); 4.9612 (50.0);
2.1801 (2.2); 1.3126 (0.7); 1.3015 (1.4); 1.2897 (1.7); 1.2798 (1.6); 1.2652 (3.6); 0.8932 (3.4); 0.8817 (7.7); 0.8697 (3.7); −0.0001 (17.0)
II-05: ¹H-NMR(599.6 MHz, CDCl3):
δ = 8.1957 (8.6); 8.1880 (8.5); 8.0835 (12.3); 8.0800 (12.8); 7.8348 (4.0); 7.8321 (4.4); 7.8195 (8.3); 7.8069 (4.3); 7.8042 (4.4);
7.7761 (3.9); 7.7721 (4.2); 7.7621 (6.7); 7.7597 (6.7); 7.7497 (4.1); 7.7456 (4.0); 7.2948 (6.5); 7.2866 (6.9); 7.2828 (6.7); 7.2740
(6.0); 7.2628 (23.5); 6.9817 (7.2); 6.9773 (7.4); 6.9676 (6.9); 6.9631 (6.9); 5.1781 (3.4); 5.1665 (10.9); 5.1549 (10.9); 5.1433 (3.4);
4.3424 (0.4); 4.3306 (0.3); 4.2776 (0.5); 4.2656 (1.4); 4.2596 (4.0); 4.2557 (4.8); 4.2477 (11.6); 4.2439 (12.5); 4.2358 (11.9);
4.2321 (12.2); 4.2240 (4.5); 4.2203 (4.1); 4.2140 (1.3); 4.2021 (0.4); 4.1278 (0.4); 4.1158 (0.4); 2.0443 (1.4); 1.6935 (42.5);
1.6819 (42.0); 1.6011 (0.4); 1.5896 (0.6); 1.5719 (39.4); 1.3895 (0.4); 1.3777 (0.8); 1.3660 (0.5); 1.3002 (1.1); 1.2766 (25.3);
1.2647 (50.0); 1.2529 (25.0); 1.2157 (0.3); 1.2037 (0.4); 0.8933 (2.0); 0.8819 (4.4); 0.8699 (2.2); −0.0001 (32.1)
II-09: ¹H-NMR(400.0 MHz, CDCl3):
δ = 8.5139 (1.5); 8.5030 (1.5); 8.4435 (1.9); 8.1949 (2.9); 8.1891 (2.9); 7.7413 (1.0); 7.7352 (1.0); 7.7205 (1.5); 7.7145 (1.5);
7.7016 (1.1); 7.6955 (1.0); 7.4858 (1.4); 7.4645 (1.6); 7.2926 (1.3); 7.2808 (1.5); 7.2718 (1.6); 7.2608 (82.8); 7.0153 (1.8); 7.0081
(1.9); 6.9942 (1.7); 6.9867 (1.7); 4.9214 (16.0); 4.3124 (1.9); 4.2945 (6.0); 4.2766 (6.0); 4.2589 (2.0); 2.9572 (0.9); 2.8845 (0.7);
1.5848 (2.3); 1.3288 (6.7); 1.3110 (13.5); 1.2932 (6.6); 0.0080 (1.2); −0.0002 (43.2); −0.0084 (1.4)
II-36: ¹H-NMR(400.6 MHz, CDCl3):
δ = 8.2052 (0.5); 8.2018 (0.7); 8.2008 (0.7); 8.1975 (0.6); 8.1931 (0.6); 8.1896 (0.7); 8.1888 (0.8); 8.1853 (0.6); 8.0862 (1.0);
8.0841 (0.8); 8.0820 (0.8); 8.0799 (1.1); 8.0781 (0.7); 7.8409 (0.6); 7.8362 (0.6); 7.8216 (0.6); 7.8175 (0.8); 7.8135 (0.6); 7.7989
(0.6); 7.7943 (0.6); 7.7832 (0.6); 7.7769 (0.6); 7.7646 (0.6); 7.7619 (0.7); 7.7583 (0.6); 7.7557 (0.6); 7.7434 (0.6); 7.7371 (0.6);
7.3055 (0.7); 7.3027 (0.7); 7.2933 (0.7); 7.2906 (0.7); 7.2862 (0.6); 7.2834 (0.6); 7.2741 (0.6); 7.2713 (0.6); 7.2622 (8.3); 6.9897
(0.7); 6.9882 (0.8); 6.9822 (0.8); 6.9807 (0.8); 6.9685 (0.7); 6.9669 (0.7); 6.9610 (0.7); 6.9594 (0.7); 5.2008 (1.5); 5.1833 (1.5);
3.7816 (16.0); 2.0454 (0.9); 1.7016 (6.2); 1.6842 (6.1); 1.5607 (4.6); 1.2596 (0.8); 0.8818 (0.8); −0.0002 (10.6)
III-01: ¹H-NMR(400.6 MHz, CDCl3):
δ = 8.2767 (2.0); 8.2745 (1.5); 8.2724 (1.6); 8.2704 (2.0); 8.2685 (1.3); 8.0783 (2.9); 8.0642 (3.0); 7.7967 (1.1); 7.7905 (1.1);
7.7784 (1.2); 7.7756 (1.3); 7.7721 (1.2); 7.7693 (1.3); 7.7572 (1.2); 7.7510 (1.2); 7.2610 (34.6); 7.1099 (1.4); 7.1083 (1.4); 7.1023
(1.4); 7.1007 (1.4); 7.0887 (1.4); 7.0872 (1.4); 7.0811 (1.4); 7.0795 (1.3); 6.8357 (1.1); 6.8328 (1.3); 6.8311 (1.5); 6.8283 (1.4);
6.8216 (1.0); 6.8187 (1.2); 6.8170 (1.6); 6.8143 (1.5); 6.7998 (2.1); 6.7966 (2.8); 6.7932 (1.6); 4.9354 (14.4); 4.3306 (1.8); 4.3128
(5.9); 4.2950 (6.0); 4.2772 (1.9); 2.0455 (0.9); 1.5450 (8.2); 1.3469 (7.6); 1.3291 (16.0); 1.3113 (7.5); 1.2641 (0.7); 1.2597 (1.0);
0.8820 (1.6); 0.8643 (0.6); 0.0080 (1.2); −0.0002 (46.5); −0.0085 (1.3)
III-02: ¹H-NMR(400.6 MHz, d₆-DMSO):
δ = 13.1557 (0.6); 8.3798 (3.0); 8.3736 (3.1); 8.1842 (4.4); 8.1701 (4.5); 8.1164 (1.3); 8.1100 (1.2); 8.0969 (1.6); 8.0952 (1.8);
8.0908 (1.6); 8.0889 (1.7); 8.0758 (1.4); 8.0695 (1.4); 7.4184 (1.9); 7.4129 (1.9); 7.3971 (1.8); 7.3917 (1.8); 7.0277 (1.4); 7.0233
(2.1); 7.0201 (1.7); 7.0137 (1.4); 7.0093 (2.2); 7.0061 (1.8); 6.9802 (3.7); 4.9454 (12.8); 4.0554 (1.1); 4.0377 (3.4); 4.0199 (3.4);
4.0021 (1.1); 3.3185 (43.8); 2.6790 (0.6); 2.6744 (1.3); 2.6698 (1.8); 2.6652 (1.3); 2.6605 (0.6); 2.5342 (1.0); 2.5294 (1.5); 2.5236
(5.5); 2.5190 (7.4); 2.5102 (97.6); 2.5056 (219.0); 2.5010 (309.1); 2.4964 (217.1); 2.4918 (96.9); 2.4596 (0.7); 2.4561 (0.7);
2.3374 (0.6); 2.3327 (1.3); 2.3281 (1.8); 2.3234 (1.3); 2.3190 (0.6); 1.9885 (16.0); 1.9082 (6.6); 1.1922 (4.7); 1.1744 (9.7); 1.1566
(4.7); 0.1458 (1.0); 0.0294 (0.5); 0.0279 (0.8); 0.0174 (0.5); 0.0150 (0.6); 0.0143 (0.7); 0.0134 (0.7); 0.0127 (0.8); 0.0119 (0.9);
0.0111 (1.0); 0.0102 (1.4); 0.0080 (9.5); 0.0064 (2.4); 0.0055 (2.6); 0.0047 (3.2); 0.0038 (4.5); −0.0002 (322.9); −0.0050
(4.7); −0.0059 (3.4); −0.0067 (2.8); −0.0085 (9.1); −0.0107 (1.2); −0.0115 (1.1); −0.0122 (0.8); −0.0130 (0.6); −0.0394 (0.6); −0.1493 (1.0)
III-07: ¹H-NMR(400.6 MHz, CDCl3):
δ = 8.2904 (1.3); 8.2861 (1.0); 8.2841 (1.3); 8.1193 (1.8); 8.1052 (1.9); 7.7981 (0.7); 7.7918 (0.7); 7.7798 (0.7); 7.7769 (0.8);
7.7735 (0.8); 7.7706 (0.8); 7.7586 (0.7); 7.7523 (0.7); 7.2633 (17.0); 7.1206 (0.9); 7.1193 (0.9); 7.1130 (0.9); 7.1117 (0.9); 7.0995
(0.8); 7.0980 (0.9); 7.0919 (0.8); 7.0905 (0.8); 6.9198 (0.8); 6.9170 (0.9); 6.9153 (0.9); 6.9126 (0.8); 6.9058 (0.7); 6.9030 (0.9);
6.9012 (0.9); 6.8985 (0.8); 6.8176 (1.2); 6.8141 (1.9); 6.8106 (1.1); 5.3011 (4.1); 4.9294 (7.5); 4.1853 (3.5); 4.1720 (3.4); 3.7992
(16.0); 2.0456 (1.8); 1.5772 (7.7); 1.2776 (0.5); 1.2597 (1.1); 1.2419 (0.5); −0.0002 (11.7)
III-08: ¹H-NMR(400.6 MHz, CDCl3):
δ = 8.5203 (1.6); 8.5065 (1.6); 8.2626 (1.2); 8.2606 (1.8); 8.2586 (1.5); 8.2565 (1.5); 8.2544 (1.9); 8.2524 (1.4); 7.7684 (1.0);
7.7622 (1.0); 7.7499 (1.2); 7.7473 (1.3); 7.7437 (1.2); 7.7410 (1.3); 7.7288 (1.2); 7.7225 (1.2); 7.2625 (28.3); 7.0724 (1.3); 7.0708
(1.4); 7.0648 (1.6); 7.0632 (1.8); 7.0589 (3.2); 7.0549 (2.3); 7.0513 (1.9); 7.0496 (2.1); 7.0471 (2.4); 7.0434 (4.2); 5.3005 (1.7);
4.9364 (13.7); 4.3247 (1.8); 4.3069 (5.9); 4.2891 (6.0); 4.2713 (2.0); 2.7729 (4.0); 1.3386 (7.7); 1.3208 (16.0); 1.3030 (7.6);
1.2847 (0.5); 0.0079 (0.7); −0.0002 (25.2); −0.0052 (0.5); −0.0085 (0.8)
III-09: ¹H-NMR(400.6 MHz, d₆-DMSO):
δ = 13.1233 (0.7); 8.5404 (6.3); 8.5364 (4.4); 8.5288 (4.4); 8.5248 (6.5); 8.3405 (3.4); 8.3342 (3.7); 8.0736 (1.5); 8.0674 (1.5);
8.0542 (1.8); 8.0525 (2.0); 8.0480 (1.9); 8.0462 (2.0); 8.0331 (1.6); 8.0268 (1.6); 7.3970 (2.3); 7.3915 (2.3); 7.3757 (2.2); 7.3702
(2.1); 7.1641 (7.8); 7.1602 (5.2); 7.1525 (5.2); 7.1485 (7.8); 5.4145 (0.7); 4.9237 (16.0); 3.6010 (1.0); 3.3361 (9.1); 2.6792 (0.6);
2.6745 (1.4); 2.6698 (1.9); 2.6653 (1.3); 2.6606 (0.6); 2.5643 (7.0); 2.5237 (5.5); 2.5190 (7.1); 2.5102 (102.6); 2.5057 (226.2);
2.5011 (317.8); 2.4965 (217.1); 2.4919 (95.5); 2.3374 (0.6); 2.3327 (1.3); 2.3281 (1.9); 2.3235 (1.3); 2.3189 (0.6); 2.1830 (0.5);
1.9083 (1.3); 1.7596 (1.2); 1.3549 (4.9); 1.2980 (1.0); 1.2584 (1.6); 1.2352 (1.8); 1.1809 (0.5); 0.8568 (0.6); 0.8404 (0.6); 0.0080
(3.3); −0.0002 (131.7); −0.0085 (3.6)
III-17: ¹H-NMR(400.6 MHz, CDCl3):
δ = 8.2900 (1.0); 8.2858 (0.8); 8.2838 (1.1); 8.2369 (1.5); 8.2229 (1.5); 7.7967 (0.5); 7.7905 (0.5); 7.7784 (0.6); 7.7756 (0.7);
7.7721 (0.6); 7.7693 (0.6); 7.7572 (0.6); 7.7509 (0.6); 7.2622 (23.5); 7.2472 (1.5); 7.2431 (1.5); 7.2425 (1.5); 7.1074 (0.7); 7.1059
(0.7); 7.0998 (0.7); 7.0984 (0.7); 7.0862 (0.6); 7.0847 (0.7); 7.0786 (0.7); 7.0771 (0.7); 6.8507 (1.2); 6.8457 (1.1); 6.8367 (1.2);
6.8318 (1.2); 5.3008 (2.4); 4.9451 (6.9); 4.4248 (0.5); 4.4071 (0.5); 4.3977 (1.4); 4.3800 (1.5); 4.3691 (1.4); 4.3554 (1.5); 4.3420
(0.6); 4.3283 (0.5); 3.7264 (5.8); 3.6838 (16.0); 2.8442 (0.7); 2.8304 (0.7); 1.5598 (1.0); 1.2166 (6.4); 1.1985 (8.8); 1.1801 (2.8);
0.0080 (0.5); −0.0002 (20.0); −0.0085 (0.6)
III-18: ¹H-NMR(400.6 MHz, CDCl3):
δ = 8.2943 (1.2); 8.2920 (0.9); 8.2880 (1.2); 8.2667 (1.9); 8.2527 (2.0); 7.7848 (0.6); 7.7785 (0.6); 7.7664 (0.7); 7.7636 (0.7);
7.7602 (0.7); 7.7574 (0.7); 7.7454 (0.6); 7.7391 (0.6); 7.2730 (2.0); 7.2718 (1.9); 7.2681 (2.3); 7.2669 (2.2); 7.2613 (66.1); 7.2571
(1.1); 7.2547 (0.6); 7.1186 (0.9); 7.1110 (0.9); 7.0974 (1.0); 7.0959 (0.9); 7.0898 (1.2); 6.8962 (1.4); 6.8913 (1.4); 6.8822 (1.4);
6.8773 (1.4); 5.3005 (5.6); 4.8910 (7.1); 4.7277 (0.8); 4.7097 (1.2); 4.6916 (0.8); 3.7897 (16.0); 2.0457 (0.9); 1.5610 (0.8); 1.5155
(6.4); 1.4976 (6.4); 1.2598 (0.8); 0.0080 (1.3); −0.0002 (43.5); −0.0085 (1.2)
III-19: ¹H-NMR(400.6 MHz, CDCl3):
δ = 8.2865 (1.7); 8.2806 (1.7); 8.2423 (2.2); 8.2284 (2.2); 7.7914 (0.6); 7.7851 (0.6); 7.7728 (0.9); 7.7702 (1.0); 7.7667 (0.9);
7.7642 (0.9); 7.7518 (0.7); 7.7456 (0.7); 7.2618 (22.0); 7.2373 (2.4); 7.2328 (2.5); 7.1055 (1.1); 7.0987 (1.1); 7.0844 (1.0); 7.0776
(1.0); 6.8606 (1.5); 6.8557 (1.5); 6.8466 (1.5); 6.8417 (1.4); 5.3007 (3.1); 4.9430 (8.9); 4.5290 (2.3); 4.5135 (4.5); 4.4980 (2.3);
3.6977 (16.0); 2.7244 (2.3); 2.7090 (4.5); 2.6934 (2.2); 1.5550 (4.1); 0.0077 (1.0); −0.0002 (18.4); −0.0085 (0.8)
III-20: ¹H-NMR(400.0 MHz, CDCl3):
δ = 8.8739 (2.0); 8.3010 (4.7); 8.2871 (7.3); 7.7875 (1.5); 7.7666 (2.3); 7.7485 (1.3); 7.5191 (0.9); 7.2606 (138.6); 7.2459 (5.6);
7.1329 (2.3); 7.1119 (2.3); 6.9969 (1.0); 6.8927 (2.9); 6.8787 (2.6); 5.3001 (6.6); 4.9916 (16.0); 4.3623 (1.9); 4.3410 (5.6); 4.3199
(5.6); 4.2984 (2.1); 3.7009 (1.1); 3.6783 (3.4); 2.3863 (1.1); 2.0480 (1.9); 1.2551 (5.1); 0.8813 (1.0); −0.0002 (86.1)
III-21: ¹H-NMR(400.6 MHz, CDCl3):
δ = 8.2761 (1.6); 8.2700 (1.7); 8.2213 (2.2); 8.2074 (2.2); 7.7718 (0.8); 7.7655 (0.8); 7.7533 (1.0); 7.7506 (1.0); 7.7470 (1.0);
7.7444 (1.0); 7.7322 (0.9); 7.7259 (0.9); 7.3382 (2.1); 7.3341 (2.2); 7.2618 (48.0); 7.0931 (1.1); 7.0869 (1.0); 7.0720 (1.1); 7.0657
(0.9); 6.8511 (1.6); 6.8462 (1.6); 6.8371 (1.6); 6.8322 (1.8); 5.3006 (10.2); 5.1282 (8.2); 5.0132 (1.4); 4.1923 (7.1); 4.1629 (1.4);
3.8196 (2.8); 3.7545 (16.0); 3.7263 (1.9); 3.6778 (1.2); 3.1780 (15.4); 3.0640 (2.4); 2.0455 (1.5); 1.5623 (2.7); 1.2775 (0.5);
1.2596 (1.2); 1.1981 (0.9); 1.1800 (0.9); 0.0079 (1.2); −0.0002 (41.9); −0.0085 (1.3)
III-22: ¹H-NMR(400.6 MHz, CDCl3):
δ = 8.2716 (1.1); 8.2695 (0.8); 8.2674 (0.9); 8.2653 (1.1); 8.2244 (1.7); 8.2233 (1.7); 8.2104 (1.8); 8.2093 (1.7); 7.7748 (0.6);
7.7685 (0.6); 7.7563 (0.7); 7.7536 (0.7); 7.7501 (0.7); 7.7473 (0.7); 7.7352 (0.6); 7.7289 (0.6); 7.2610 (18.5); 7.2044 (1.7); 7.2033
(1.8); 7.1995 (1.8); 7.1984 (1.7); 7.1034 (0.8); 7.1018 (0.8); 7.0957 (0.8); 7.0942 (0.8); 7.0822 (0.7); 7.0807 (0.8); 7.0746 (0.8);
7.0731 (0.7); 6.8187 (1.6); 6.8138 (1.5); 6.8047 (1.5); 6.7998 (1.5); 5.3003 (4.6); 5.2557 (1.5); 5.2383 (1.5); 3.8171 (16.0); 1.7238
(6.1); 1.7064 (6.0); 1.5446 (5.7); 0.0079 (0.8); −0.0002 (29.6); −0.0044 (0.7); −0.0085 (0.9)
III-23: ¹H-NMR(400.6 MHz, d₆-DMSO):
δ = 8.3828 (3.2); 8.3765 (3.4); 8.3304 (5.1); 8.3293 (5.1); 8.3165 (5.3); 8.3153 (5.3); 8.1194 (1.3); 8.1132 (1.3); 8.1000 (1.7);
8.0982 (1.8); 8.0938 (1.7); 8.0920 (1.8); 8.0789 (1.4); 8.0726 (1.4); 7.4231 (1.9); 7.4175 (2.0); 7.4017 (2.0); 7.3962 (1.8); 7.3949
(1.8); 7.3413 (5.0); 7.3402 (5.4); 7.3365 (5.5); 7.3353 (5.0); 7.0466 (4.6); 7.0418 (4.3); 7.0327 (4.5); 7.0278 (4.5); 4.9455 (13.8);
4.0555 (1.0); 4.0377 (3.3); 4.0200 (3.4); 4.0022 (1.1); 3.3197 (14.5); 2.6746 (0.7); 2.6700 (1.0); 2.6654 (0.7); 2.5238 (2.6); 2.5191
(3.6); 2.5104 (53.3); 2.5058 (117.6); 2.5012 (165.8); 2.4966 (113.7); 2.4920 (50.1); 2.3329 (0.7); 2.3282 (1.0); 2.3236 (0.7);
1.9886 (16.0); 1.9084 (6.4); 1.1922 (4.8); 1.1744 (9.8); 1.1566 (4.6); 0.0079 (3.5); 0.0063 (0.8); 0.0055 (0.8); 0.0047 (1.0); 0.0039
(1.5); 0.0030 (2.6); 0.0022 (4.6); −0.0002 (126.0); −0.0027 (5.4); −0.0035 (3.7); −0.0043 (2.2); −0.0052 (1.6); −0.0060 (1.4); −0.0068
(1.2); −0.0085 (3.7); −0.0098 (0.8); −0.0107 (0.6)
III-24: ¹H-NMR(400.6 MHz, CDCl3):
δ = 8.2774 (2.2); 8.2755 (1.7); 8.2730 (1.8); 8.2712 (2.3); 8.2274 (3.4); 8.2135 (3.5); 7.7848 (1.1); 7.7785 (1.1); 7.7664 (1.2);
7.7637 (1.4); 7.7601 (1.3); 7.7574 (1.3); 7.7453 (1.2); 7.7390 (1.2); 7.2659 (5.4); 7.2566 (3.4); 7.2559 (3.3); 7.2518 (3.7); 7.1090
(1.5); 7.1076 (1.5); 7.1014 (1.6); 7.1000 (1.5); 7.0878 (1.4); 7.0865 (1.4); 7.0802 (1.5); 7.0789 (1.4); 6.8223 (2.7); 6.8174 (2.6);
6.8083 (2.6); 6.8034 (2.6); 5.3015 (10.6); 4.9365 (15.5); 4.3343 (2.0); 4.3165 (6.4); 4.2987 (6.4); 4.2809 (2.1); 1.5976 (1.8);
1.3536 (7.8); 1.3358 (16.0); 1.3180 (7.5); −0.0002 (7.7)
III-25: ¹H-NMR(400.6 MHz, d₆-DMSO):
δ = 12.6699 (1.3); 8.5145 (2.9); 8.4960 (3.0); 8.3741 (4.4); 8.3678 (4.5); 8.1583 (6.5); 8.1441 (6.8); 8.1147 (1.8); 8.1085 (1.8);
8.0953 (2.3); 8.0936 (2.5); 8.0891 (2.2); 8.0873 (2.4); 8.0742 (1.9); 8.0679 (1.9); 7.4245 (2.7); 7.4190 (2.7); 7.4046 (2.4); 7.4032
(2.6); 7.3977 (2.6); 7.3964 (2.4); 7.0636 (4.8); 7.0622 (4.9); 7.0059 (2.4); 7.0027 (3.1); 6.9984 (2.1); 6.9918 (2.4); 6.9886 (3.0);
6.9843 (2.1); 6.8707 (0.7); 4.8530 (15.4); 4.3384 (0.5); 4.3203 (2.6); 4.3020 (4.1); 4.2836 (2.7); 4.2656 (0.5); 3.6216 (0.9); 3.6177
(5.2); 3.6155 (2.9); 3.6114 (2.7); 3.6093 (2.0); 3.6074 (3.6); 3.6011 (12.2); 3.5989 (4.2); 3.5949 (3.8); 3.5930 (2.0); 3.5908 (2.7);
3.5866 (2.7); 3.5845 (5.4); 3.5806 (0.9); 3.3208 (25.7); 3.2024 (1.4); 2.6749 (0.6); 2.6703 (0.9); 2.6657 (0.6); 2.5409 (1.9); 2.5241
(2.4); 2.5195 (3.2); 2.5107 (48.1); 2.5061 (105.8); 2.5015 (148.8); 2.4969 (102.1); 2.4923 (44.6); 2.3332 (0.6); 2.3286 (0.9);
2.3239 (0.6); 2.1829 (1.2); 1.9949 (1.1); 1.9086 (0.8); 1.7809 (0.8); 1.7763 (5.3); 1.7686 (4.2); 1.7642 (2.5); 1.7597 (15.2); 1.7549
(2.6); 1.7507 (4.1); 1.7431 (5.2); 1.7385 (0.8); 1.3552 (11.4); 1.3254 (16.0); 1.3072 (15.9); 1.2363 (0.8); 1.0897 (0.6); 1.0743 (0.6);
0.0080 (1.4); 0.0039 (0.7); 0.0022 (1.9); −0.0002 (46.9); −0.0027 (1.9); −0.0034 (1.2); −0.0043 (0.6); −0.0085 (1.2)
III-26: ¹H-NMR(400.6 MHz, d₆-DMSO):
δ = 12.6543 (1.2); 8.4833 (1.4); 8.4686 (2.9); 8.4537 (1.4); 8.3683 (4.3); 8.3620 (4.4); 8.1613 (6.3); 8.1472 (6.6); 8.1062 (1.7);
8.0999 (1.6); 8.0850 (2.4); 8.0804 (2.2); 8.0788 (2.3); 8.0656 (1.8); 8.0594 (1.8); 7.4229 (2.7); 7.4172 (2.6); 7.4016 (2.6); 7.3959
(2.5); 7.0825 (5.1); 7.0259 (2.4); 7.0227 (3.1); 7.0186 (2.0); 7.0118 (2.3); 7.0086 (3.0); 7.0044 (2.0); 4.8783 (16.0); 3.8523 (8.0);
3.8376 (8.1); 3.6176 (0.8); 3.6072 (0.6); 3.6010 (1.9); 3.5948 (0.7); 3.5865 (0.5); 3.5844 (0.8); 3.3207 (28.0); 2.6747 (0.7); 2.6702
(1.0); 2.6657 (0.7); 2.5239 (2.7); 2.5193 (4.1); 2.5104 (54.6); 2.5059 (116.9); 2.5013 (161.6); 2.4968 (111.6); 2.4922 (49.2);
2.4737 (0.6); 2.4692 (0.7); 2.3331 (0.7); 2.3285 (1.0); 2.3238 (0.7); 1.7762 (0.8); 1.7686 (0.7); 1.7596 (2.4); 1.7506 (0.7); 1.7431
(0.8); 1.3550 (3.8); 0.0080 (1.2); −0.0002 (45.9); −0.0085 (1.3)
III-27: ¹H-NMR(400.6 MHz, CDCl3):
δ = 8.2873 (1.2); 8.2853 (0.9); 8.2831 (0.9); 8.2810 (1.2); 8.0864 (1.6); 8.0724 (1.6); 7.8080 (0.6); 7.8017 (0.6); 7.7896 (0.7);
7.7869 (0.8); 7.7834 (0.7); 7.7806 (0.7); 7.7685 (0.7); 7.7622 (0.7); 7.2627 (17.0); 7.1099 (0.8); 7.1084 (0.8); 7.1023 (0.8); 7.1008
(0.8); 7.0888 (0.8); 7.0872 (0.8); 7.0811 (0.8); 7.0796 (0.8); 6.8565 (0.6); 6.8537 (0.8); 6.8520 (0.9); 6.8492 (0.8); 6.8425 (0.6);
6.8396 (0.7); 6.8379 (0.9); 6.8351 (0.8); 6.8129 (1.2); 6.8096 (1.6); 6.8063 (0.9); 5.3009 (2.2); 4.9395 (8.0); 4.5278 (2.0); 4.5123
(3.8); 4.4969 (2.0); 3.6987 (16.0); 2.7208 (1.9); 2.7054 (3.7); 2.6899 (1.9); 1.5644 (10.0); −0.0002 (11.8)
III-28: ¹H-NMR(400.6 MHz, CDCl3):
δ = 8.2929 (1.1); 8.2908 (0.9); 8.2885 (0.9); 8.2867 (1.2); 8.1171 (1.5); 8.1031 (1.5); 7.8021 (0.6); 7.7958 (0.6); 7.7838 (0.7);
7.7809 (0.7); 7.7775 (0.7); 7.7747 (0.7); 7.7626 (0.6); 7.7563 (0.6); 7.2643 (11.6); 7.1225 (0.8); 7.1211 (0.8); 7.1149 (0.9); 7.1135
(0.8); 7.1013 (1.0); 7.0999 (1.0); 7.0938 (1.1); 7.0924 (1.0); 6.9154 (0.7); 6.9126 (0.8); 6.9108 (0.8); 6.9082 (0.7); 6.9013 (0.6);
6.8985 (0.8); 6.8968 (0.8); 6.8941 (0.7); 6.8166 (1.0); 6.8132 (1.6); 6.8097 (1.0); 5.3014 (4.0); 4.8908 (7.1); 4.7269 (0.8); 4.7088
(1.2); 4.6908 (0.8); 3.7883 (16.0); 2.0458 (1.6); 1.5928 (5.1); 1.5143 (6.4); 1.4964 (6.3); 1.2777 (0.5); 1.2599 (1.1); −0.0002 (8.1)
I-127: ¹H-NMR(400.6 MHz, CDCl3):
δ = 8.2376 (2.6); 8.2356 (2.7); 8.2259 (2.7); 8.2239 (2.7); 8.1668 (4.8); 8.1539 (5.0); 7.4907 (1.7); 7.4871 (1.7); 7.4701 (2.5);
7.4668 (3.3); 7.4637 (2.0); 7.4467 (2.2); 7.4430 (2.1); 7.3144 (2.0); 7.3054 (2.4); 7.3028 (2.2); 7.2938 (3.6); 7.2848 (1.8); 7.2822
(1.8); 7.2732 (1.6); 7.2621 (22.4); 7.0469 (2.1); 7.0429 (3.0); 7.0390 (2.1); 7.0339 (2.2); 7.0300 (3.0); 7.0261 (2.0); 6.8544 (5.3);
5.3339 (1.2); 5.3165 (5.0); 5.2991 (5.2); 5.2819 (1.3); 2.2717 (0.5); 1.6805 (16.0); 1.6702 (2.1); 1.6631 (15.9); 1.6527 (2.0);
1.6361 (1.4); 1.6333 (1.3); 1.6241 (0.9); 1.6176 (2.9); 1.6106 (1.0); 1.6039 (1.1); 1.6011 (1.3); 1.5995 (1.4); 1.5968 (0.9); 1.5832
(0.9); 1.4321 (5.3); 1.2646 (1.8); 0.8987 (1.0); 0.8818 (3.6); 0.8642 (1.4); 0.8268 (1.2); 0.8212 (6.2); 0.8154 (8.6); 0.8143 (8.8);
0.8089 (4.8); 0.8077 (4.7); 0.8033 (5.4); 0.7935 (10.3); 0.7764 (0.5); 0.0080 (0.9); −0.0002 (32.1); −0.0085 (1.0)
I-128: ¹H-NMR(400.6 MHz, CDCl3):
δ = 8.2263 (1.2); 8.2244 (1.4); 8.2226 (1.4); 8.2208 (1.2); 8.2146 (1.3); 8.2127 (1.4); 8.2110 (1.4); 8.2091 (1.2); 8.1564 (2.4);
8.1434 (2.5); 7.5008 (1.1); 7.4971 (1.1); 7.4802 (1.4); 7.4767 (1.8); 7.4737 (1.1); 7.4568 (1.4); 7.4531 (1.3); 7.3167 (1.3); 7.3078
(1.4); 7.3051 (1.3); 7.2961 (2.3); 7.2872 (1.1); 7.2845 (1.1); 7.2755 (1.0); 7.2623 (15.3); 7.0528 (1.1); 7.0492 (1.4); 7.0482 (1.5);
7.0448 (1.1); 7.0398 (1.1); 7.0363 (1.4); 7.0353 (1.4); 7.0318 (1.1); 6.8644 (1.3); 6.8629 (1.6); 6.8597 (2.6); 6.8565 (1.5); 6.8550
(1.3); 4.8811 (14.3); 4.2732 (1.8); 4.2554 (5.6); 4.2375 (5.7); 4.2197 (1.8); 1.6619 (0.5); 1.6607 (0.6); 1.6552 (0.8); 1.6479 (0.6);
1.6412 (1.8); 1.6334 (0.6); 1.6280 (0.6); 1.6262 (0.6); 1.6212 (0.8); 1.6068 (0.5); 1.5698 (3.5); 1.2992 (7.6); 1.2814 (16.0); 1.2636
(7.6); 1.2596 (0.5); 0.8547 (0.5); 0.8516 (1.2); 0.8490 (1.5); 0.8466 (3.1); 0.8430 (2.4); 0.8382 (3.6); 0.8340 (4.7); 0.8328 (4.2);
0.8285 (2.8); 0.8185 (2.1); 0.8143 (2.8); 0.8110 (1.4); 0.8083 (1.4); 0.0079 (0.5); −0.0002 (20.0); −0.0085 (0.6)
I-129: ¹H-NMR(400.6 MHz, CDCl3):
δ = 8.2292 (0.8); 8.2274 (0.9); 8.2256 (0.9); 8.2238 (0.8); 8.2176 (0.8); 8.2157 (1.0); 8.2139 (0.9); 8.2121 (0.8); 8.1574 (1.6);
8.1445 (1.6); 7.5023 (0.7); 7.4986 (0.7); 7.4817 (0.9); 7.4783 (1.1); 7.4752 (0.8); 7.4583 (0.8); 7.4546 (0.8); 7.3199 (0.8); 7.3110
(0.9); 7.3083 (0.9); 7.2993 (1.5); 7.2903 (0.7); 7.2876 (0.7); 7.2787 (0.6); 7.2614 (18.2); 7.0522 (0.7); 7.0478 (1.0); 7.0442 (0.8);
7.0393 (0.7); 7.0356 (0.9); 7.0312 (0.7); 6.8626 (1.0); 6.8594 (1.7); 6.8562 (1.0); 6.8548 (0.9); 4.8996 (8.5); 3.7846 (16.0); 1.6567
(0.5); 1.6405 (1.1); 1.6226 (0.6); 1.5569 (7.6); 0.8341 (11.1); 0.8245 (0.8); 0.8226 (0.8); 0.8181 (2.5); 0.8160 (2.6); 0.8148 (2.6);
0.8114 (0.8); 0.8095 (0.8); 0.0080 (0.6); −0.0002 (23.4); −0.0085 (0.6)
I-130: ¹H-NMR(400.6 MHz, CDCl3):
δ = 8.2560 (0.9); 8.2462 (0.9); 8.2030 (1.2); 8.1900 (1.3); 7.5471 (0.6); 7.5435 (0.6); 7.5264 (0.9); 7.5230 (1.1); 7.5206 (0.8);
7.5034 (0.8); 7.4997 (0.8); 7.3891 (0.6); 7.3800 (0.7); 7.3777 (0.7); 7.3685 (1.1); 7.3594 (0.6); 7.3570 (0.6); 7.2620 (7.4); 7.1054
(0.7); 7.1016 (1.1); 7.0978 (0.8); 7.0925 (0.7); 7.0886 (1.1); 7.0847 (0.7); 6.9137 (1.6); 4.9545 (8.4); 3.8005 (16.0); 1.5626
(2.3); −0.0002 (10.9)
I-131: ¹H-NMR(400.6 MHz, CDCl3):
δ = 8.2750 (1.6); 8.2733 (1.8); 8.2713 (1.9); 8.2697 (1.7); 8.2633 (1.7); 8.2615 (1.9); 8.2596 (1.9); 8.2581 (1.6); 8.1740 (3.2);
8.1610 (3.3); 7.4993 (1.2); 7.4956 (1.3); 7.4787 (1.7); 7.4752 (2.1); 7.4723 (1.5); 7.4554 (1.6); 7.4517 (1.6); 7.3385 (1.6); 7.3294
(1.8); 7.3268 (1.7); 7.3178 (2.8); 7.3088 (1.3); 7.3062 (1.3); 7.2972 (1.2); 7.2626 (13.9); 7.0533 (1.3); 7.0491 (2.0); 7.0454 (1.4);
7.0403 (1.4); 7.0363 (2.0); 7.0324 (1.4); 6.8644 (2.1); 6.8613 (3.4); 6.8583 (2.1); 4.9420 (16.0); 1.6519 (0.8); 1.6462 (1.0); 1.6399
(0.7); 1.6322 (2.0); 1.6256 (0.7); 1.6192 (0.7); 1.6176 (0.9); 1.6120 (1.0); 1.5980 (0.6); 1.4321 (1.6); 0.8307 (1.6); 0.8249 (3.2);
0.8204 (2.5); 0.8090 (3.6); 0.8046 (6.8); 0.8009 (4.3); 0.7945 (2.8); 0.7907 (3.9); 0.7878 (1.9); 0.7854 (1.8); −0.0002
(19.4); −0.0085 (0.6)
I-132: ¹H-NMR(400.6 MHz, CDCl3):
δ = 8.1722 (0.7); 8.1705 (0.8); 8.1686 (0.8); 8.1669 (0.7); 8.1606 (0.7); 8.1587 (0.8); 8.1570 (0.8); 8.1552 (0.8); 8.1489 (1.4);
8.1360 (1.4); 7.5042 (0.6); 7.5005 (0.6); 7.4837 (0.8); 7.4802 (1.1); 7.4769 (0.7); 7.4600 (0.7); 7.4563 (0.7); 7.2932 (0.7); 7.2843
(0.8); 7.2815 (0.7); 7.2726 (1.3); 7.2620 (9.4); 7.2521 (0.6); 7.0450 (0.6); 7.0406 (0.9); 7.0370 (0.7); 7.0321 (0.6); 7.0285 (0.8);
7.0276 (0.8); 7.0240 (0.6); 6.8588 (0.9); 6.8556 (1.5); 6.8525 (0.9); 6.8510 (0.8); 5.2600 (1.5); 5.2426 (1.5); 3.7498 (16.0); 1.6473
(0.5); 1.6399 (0.8); 1.6351 (6.4); 1.6295 (0.8); 1.6269 (0.8); 1.6178 (6.5); 1.6134 (0.7); 1.6057 (0.6); 1.5688 (3.1); 0.8666 (0.5);
0.8633 (1.0); 0.8603 (1.2); 0.8569 (0.8); 0.8533 (1.2); 0.8502 (1.0); 0.8471 (1.2); 0.8438 (0.6); 0.8400 (1.0); 0.8288 (1.1); 0.8225
(1.0); 0.8208 (1.1); 0.8165 (0.7); 0.8146 (0.7); 0.8076 (0.9); 0.8009 (1.1); 0.7950 (0.7); −0.0002 (12.1)
I-133: ¹H-NMR(400.6 MHz, CDCl3):
δ = 8.2092 (1.8); 8.1958 (2.2); 8.1924 (2.5); 8.1794 (2.4); 7.5432 (1.1); 7.5396 (1.1); 7.5224 (1.6); 7.5195 (2.2); 7.5165 (1.4);
7.4993 (1.4); 7.4957 (1.4); 7.3639 (1.2); 7.3549 (1.4); 7.3525 (1.3); 7.3434 (2.1); 7.3343 (1.1); 7.3318 (1.1); 7.3228 (0.9); 7.2615
(15.6); 7.0990 (1.3); 7.0951 (2.2); 7.0913 (1.4); 7.0860 (1.3); 7.0822 (2.1); 7.0784 (1.3); 6.9101 (3.1); 5.2722 (0.9); 5.2548 (3.2);
5.2374 (3.2); 5.2201 (0.9); 4.2525 (1.7); 4.2347 (5.4); 4.2169 (5.7); 4.1992 (1.9); 1.6942 (12.7); 1.6768 (12.6); 1.5601 (3.1);
1.2695 (7.9); 1.2517 (16.0); 1.2339 (7.6); 0.0078 (0.5); −0.0002 (22.5); −0.0085 (0.7)
I-135: ¹H-NMR(400.6 MHz, CDCl3):
δ = 8.1737 (1.3); 8.1718 (1.5); 8.1701 (1.5); 8.1682 (1.4); 8.1621 (1.4); 8.1602 (1.6); 8.1584 (1.6); 8.1566 (1.4); 8.1475 (2.7);
8.1345 (2.8); 7.4992 (1.1); 7.4955 (1.2); 7.4786 (1.4); 7.4753 (2.1); 7.4719 (1.3); 7.4551 (1.4); 7.4513 (1.3); 7.2902 (1.4); 7.2813
(1.5); 7.2785 (1.4); 7.2696 (2.5); 7.2611 (39.8); 7.2491 (1.1); 7.0454 (1.2); 7.0409 (1.6); 7.0374 (1.2); 7.0324 (1.2); 7.0289 (1.6);
7.0244 (1.2); 6.8583 (1.7); 6.8551 (2.8); 6.8520 (1.8); 6.8505 (1.4); 5.2599 (0.8); 5.2426 (3.0); 5.2252 (3.1); 5.2079 (0.8); 4.2326
(2.0); 4.2148 (6.6); 4.1970 (6.7); 4.1793 (2.2); 1.6489 (0.9); 1.6412 (1.1); 1.6313 (12.0); 1.6199 (1.0); 1.6140 (12.3); 1.6071 (1.4);
1.5938 (0.6); 1.5546 (16.0); 1.2635 (7.5); 1.2458 (15.7); 1.2279 (7.3); 0.8906 (0.5); 0.8883 (0.8); 0.8793 (1.0); 0.8766 (1.6);
0.8734 (2.5); 0.8652 (1.7); 0.8634 (1.7); 0.8604 (2.4); 0.8580 (1.4); 0.8520 (1.2); 0.8305 (0.8); 0.8285 (1.1); 0.8241 (0.7); 0.8191
(2.4); 0.8153 (1.9); 0.8075 (1.0); 0.8047 (1.0); 0.7987 (2.2); 0.7936 (1.7); 0.7866 (0.6); 0.0079 (1.3); −0.0002 (49.7); −0.0085 (1.5)
I-141: ¹H-NMR(400.6 MHz, CDCl3):
δ = 8.5112 (1.0); 8.3275 (1.2); 8.2270 (1.5); 8.2155 (1.5); 7.5664 (1.0); 7.5467 (2.0); 7.5432 (1.8); 7.5259 (1.6); 7.5240 (1.6);
7.5224 (1.7); 7.5205 (1.3); 7.5032 (1.4); 7.4996 (1.3); 7.3920 (1.1); 7.3828 (1.3); 7.3806 (1.2); 7.3713 (1.9); 7.3621 (1.0); 7.3598
(1.0); 7.3505 (0.8); 7.2610 (20.9); 4.9413 (14.8); 4.2908 (2.0); 4.2730 (6.3); 4.2552 (6.4); 4.2374 (2.1); 2.7814 (1.8); 1.3027 (7.7);
1.2849 (16.0); 1.2671 (7.6); 0.0080 (1.0); −0.0002 (29.8); −0.0085 (0.9)
I-142: ¹H-NMR(400.6 MHz, CDCl3):
δ = 8.4037 (1.8); 8.3968 (1.8); 8.3139 (0.9); 8.3100 (1.8); 8.3061 (1.0); 8.1684 (0.7); 8.1665 (0.8); 8.1648 (0.9); 8.1630 (0.8);
8.1568 (0.8); 8.1549 (0.9); 8.1531 (0.9); 8.1513 (0.8); 7.4838 (0.6); 7.4801 (0.6); 7.4632 (0.8); 7.4598 (1.2); 7.4564 (0.7); 7.4395
(0.8); 7.4358 (0.8); 7.4252 (0.7); 7.4209 (0.7); 7.4183 (0.7); 7.4140 (0.7); 7.4025 (0.7); 7.3982 (0.8); 7.3956 (0.7); 7.3913 (0.7);
7.2710 (0.8); 7.2625 (10.7); 7.2595 (1.1); 7.2505 (1.4); 7.2416 (0.6); 7.2388 (0.7); 7.2300 (0.6); 5.2366 (1.6); 5.2193 (1.6); 4.4686
(0.6); 4.4572 (0.8); 4.4408 (1.7); 4.4303 (0.8); 4.4244 (0.8); 4.4142 (1.7); 4.3983 (0.8); 4.3865 (0.6); 3.6689 (16.0); 2.6727 (1.5);
2.6563 (3.0); 2.6403 (1.4); 1.6245 (6.0); 1.6071 (6.3); 1.5994 (0.8); 1.5860 (3.7); 1.5782 (0.6); 1.5725 (0.6); 1.5652 (0.6); 0.8330
(1.0); 0.8290 (1.4); 0.8210 (1.5); 0.8158 (1.2); 0.8083 (0.8); 0.7994 (1.1); 0.7917 (1.2); 0.7872 (0.8); 0.7785 (0.7); 0.7713 (1.3);
0.7658 (0.9); −0.0002 (13.9)
I-143: ¹H-NMR(400.6 MHz, CDCl3):
δ = 8.4390 (3.2); 8.4321 (3.3); 8.2812 (2.0); 8.2776 (3.4); 8.2741 (2.0); 7.5874 (1.0); 7.5723 (1.1); 7.5659 (1.6); 7.5509 (1.5);
7.5455 (1.3); 7.5305 (1.0); 7.4219 (1.2); 7.4175 (1.3); 7.4151 (1.4); 7.4107 (1.2); 7.3998 (1.2); 7.3954 (1.4); 7.3930 (1.4); 7.3886
(1.2); 7.2625 (13.3); 6.8936 (1.2); 6.8873 (1.3); 6.8843 (1.3); 6.8780 (1.2); 6.8717 (1.1); 6.8655 (1.2); 6.8625 (1.2); 6.8562 (1.1);
5.1539 (0.7); 5.1501 (2.2); 5.1460 (3.4); 5.1420 (2.4); 5.0559 (2.5); 5.0537 (3.1); 5.0516 (3.1); 5.0494 (2.4); 4.9412 (0.6); 4.9182
(1.2); 4.9109 (16.0); 4.2887 (2.2); 4.2708 (6.4); 4.2665 (1.3); 4.2530 (6.3); 4.2486 (1.3); 4.2352 (2.1); 1.9291 (8.0); 1.9270 (11.3);
1.9258 (11.6); 1.9236 (8.8); 1.5869 (4.1); 1.3102 (7.4); 1.3048 (1.4); 1.2924 (15.1); 1.2868 (1.9); 1.2746 (7.3); 1.2689 (1.0);
0.0079 (0.6); −0.0002 (18.3); −0.0084 (0.6)
I-147: ¹H-NMR(400.6 MHz, CDCl3):
δ = 8.5479 (0.6); 8.3498 (0.7); 8.2082 (1.0); 8.2061 (1.1); 8.2038 (1.2); 8.2016 (1.1); 8.1961 (1.1); 8.1938 (1.2); 8.1919 (1.2);
8.1896 (1.0); 7.9352 (0.7); 7.9306 (0.7); 7.9175 (0.6); 7.9147 (1.8); 7.9102 (1.8); 7.8972 (2.1); 7.8928 (2.1); 7.8876 (1.8); 7.8852
(2.2); 7.8842 (2.4); 7.8817 (1.9); 7.8672 (0.7); 7.8650 (0.8); 7.5403 (0.8); 7.5360 (0.9); 7.5336 (1.0); 7.5293 (0.8); 7.5185 (0.8);
7.5142 (1.0); 7.5117 (0.9); 7.5075 (0.8); 7.3437 (1.2); 7.3401 (1.2); 7.3316 (1.2); 7.3277 (1.3); 7.3263 (1.4); 7.3225 (1.1); 7.3142
(1.1); 7.3104 (1.1); 7.2631 (11.1); 5.3008 (10.8); 4.4158 (16.0); 4.2758 (2.1); 4.2580 (6.7); 4.2401 (6.8); 4.2223 (2.2); 1.5815 (1.8);
1.2842 (7.5); 1.2664 (15.6); 1.2485 (7.3); 0.0702 (1.4); −0.0002 (15.8)
I-148: ¹H-NMR(400.6 MHz, d₆-DMSO):
δ = 12.9537 (2.0); 8.6263 (5.6); 8.6193 (5.6); 8.3765 (3.0); 8.3725 (5.6); 8.3686 (2.7); 8.1359 (1.2); 8.1203 (1.3); 8.1138 (2.4);
8.0982 (2.4); 8.0917 (1.3); 8.0761 (1.2); 7.8388 (1.8); 7.8344 (1.9); 7.8319 (1.9); 7.8275 (1.6); 7.8148 (1.9); 7.8104 (2.1); 7.8079
(1.8); 7.8035 (1.6); 7.3160 (1.3); 7.3096 (1.8); 7.3078 (1.7); 7.3013 (1.4); 7.2939 (1.3); 7.2875 (1.6); 7.2857 (1.5); 7.2794 (1.1);
4.7878 (16.0); 3.3202 (12.8); 2.6706 (0.5); 2.5245 (1.4); 2.5197 (2.2); 2.5110 (29.9); 2.5065 (65.8); 2.5018 (91.4); 2.4972 (63.6);
2.4926 (27.7); 1.7598 (0.6); 1.7055 (0.6); 1.6925 (1.4); 1.6845 (1.4); 1.6796 (0.9); 1.6715 (2.9); 1.6637 (0.8); 1.6583 (1.5); 1.6504
(1.5); 1.6374 (0.7); 1.3558 (2.8); 0.7629 (1.4); 0.7519 (3.0); 0.7465 (4.0); 0.7419 (1.8); 0.7373 (2.3); 0.7309 (3.2); 0.7251 (3.8);
0.7164 (1.9); 0.6414 (2.0); 0.6324 (4.1); 0.6276 (4.2); 0.6196 (4.0); 0.6141 (3.4); 0.6030 (1.3); 0.0080 (1.5); −0.0002
(51.9); −0.0085 (1.4)
I-149: ¹H-NMR(400.6 MHz, CDCl3):
δ = 8.4521 (2.0); 8.4454 (2.0); 8.3218 (2.2); 7.5768 (1.0); 7.5618 (1.0); 7.5561 (1.4); 7.5411 (1.5); 7.5347 (1.1); 7.5197 (1.1);
7.4614 (0.9); 7.4570 (1.1); 7.4546 (1.1); 7.4503 (1.0); 7.4390 (1.0); 7.4347 (1.1); 7.4322 (1.1); 7.4279 (0.9); 7.2621 (13.7); 6.8718
(1.1); 6.8656 (1.2); 6.8624 (1.2); 6.8563 (1.1); 6.8500 (1.0); 6.8439 (1.1); 6.8407 (1.1); 6.8345 (1.0); 4.8702 (15.8); 4.2824 (2.0);
4.2646 (6.2); 4.2468 (6.2); 4.2290 (2.0); 1.6001 (0.7); 1.5937 (1.0); 1.5865 (0.6); 1.5801 (1.9); 1.5694 (3.4); 1.5596 (1.1); 1.5457
(0.6); 1.3099 (7.8); 1.2921 (16.0); 1.2743 (7.6); 0.8819 (0.5); 0.8266 (1.4); 0.8209 (3.2); 0.8166 (2.5); 0.8137 (1.8); 0.8081 (4.5);
0.8028 (4.3); 0.7985 (1.6); 0.7881 (2.1); 0.7824 (2.5); 0.7770 (1.7); 0.0080 (0.6); −0.0002 (20.9); −0.0085 (0.7)
I-150: ¹H-NMR(400.6 MHz, CDCl3):
δ = 8.5332 (3.1); 8.5263 (3.1); 8.3396 (1.7); 8.3359 (3.1); 8.3322 (1.7); 8.1937 (1.3); 8.1916 (1.4); 8.1892 (1.5); 8.1871 (1.4);
8.1816 (1.3); 8.1794 (1.4); 8.1772 (1.5); 8.1749 (1.3); 7.9220 (0.9); 7.9174 (0.8); 7.9039 (0.9); 7.9015 (1.9); 7.8996 (1.0); 7.8969
(1.8); 7.8835 (2.1); 7.8790 (2.0); 7.8648 (2.0); 7.8618 (3.0); 7.8594 (2.2); 7.8443 (1.0); 7.8419 (1.2); 7.8389 (0.7); 7.5160 (1.3);
7.5116 (1.4); 7.5091 (1.4); 7.5047 (1.3); 7.4940 (1.3); 7.4896 (1.4); 7.4872 (1.3); 7.4828 (1.2); 7.3154 (1.4); 7.3122 (1.4); 7.3034
(1.4); 7.3000 (1.4); 7.2975 (1.5); 7.2943 (1.4); 7.2855 (1.4); 7.2822 (1.3); 7.2633 (11.8); 5.3005 (11.4); 4.4155 (10.2); 4.4143
(10.4); 4.2810 (1.9); 4.2631 (6.1); 4.2453 (6.3); 4.2275 (2.0); 1.5970 (0.7); 1.2959 (7.6); 1.2781 (16.0); 1.2602 (7.5); 0.0700 (0.9);
0.0080 (0.5); −0.0002 (17.9)
I-151: ¹H-NMR(400.6 MHz, CDCl3):
δ = 8.4942 (1.7); 8.4873 (1.7); 8.3401 (0.9); 8.3395 (0.9); 8.3358 (1.6); 8.3322 (0.9); 8.1010 (0.7); 8.0989 (0.7); 8.0965 (0.8);
8.0943 (0.7); 8.0890 (0.7); 8.0866 (0.8); 8.0844 (0.8); 8.0821 (0.7); 7.8174 (1.1); 7.8158 (0.5); 7.8128 (1.0); 7.7997 (1.2); 7.7951
(1.2); 7.7840 (1.2); 7.7816 (1.4); 7.7808 (1.5); 7.7783 (1.2); 7.7634 (0.5); 7.7611 (0.6); 7.7602 (0.5); 7.5019 (0.7); 7.4976 (0.8);
7.4951 (0.8); 7.4907 (0.7); 7.4796 (0.7); 7.4752 (0.8); 7.4726 (0.7); 7.4683 (0.7); 7.2626 (5.6); 7.1844 (0.8); 7.1810 (0.8); 7.1722
(0.8); 7.1687 (0.8); 7.1667 (0.9); 7.1633 (0.7); 7.1546 (0.8); 7.1511 (0.7); 3.9599 (8.7); 3.7902 (16.0); −0.0002 (8.4)
I-153: ¹H-NMR(400.0 MHz, d₆-DMSO):
δ = 12.7540 (0.8); 8.5944 (5.3); 8.5875 (5.3); 8.3680 (3.0); 8.3638 (5.5); 8.3598 (2.9); 8.1051 (2.0); 8.1032 (2.3); 8.1006 (2.5);
8.0986 (2.3); 8.0931 (2.1); 8.0911 (2.5); 8.0885 (2.5); 8.0865 (2.2); 7.9877 (1.7); 7.9829 (1.5); 7.9689 (2.3); 7.9671 (2.4); 7.9643
(2.0); 7.9624 (2.0); 7.9486 (2.2); 7.9438 (1.8); 7.7980 (1.9); 7.7937 (2.0); 7.7910 (2.0); 7.7868 (1.7); 7.7735 (2.0); 7.7693 (2.2);
7.7666 (1.9); 7.7623 (1.8); 7.7521 (4.0); 7.7336 (2.1); 7.7315 (3.4); 7.7196 (0.5); 7.2862 (2.1); 7.2838 (2.1); 7.2740 (2.0); 7.2716
(2.2); 7.2677 (2.2); 7.2653 (1.9); 7.2555 (2.0); 7.2530 (1.9); 5.7562 (7.2); 4.1401 (1.1); 4.1223 (1.1); 4.0291 (2.1); 3.9771 (16.0);
3.3198 (6.2); 2.6706 (0.5); 2.5241 (1.3); 2.5194 (1.9); 2.5106 (29.6); 2.5061 (65.1); 2.5015 (91.0); 2.4969 (64.5); 2.4924 (29.2);
1.6965 (0.5); 1.6833 (1.2); 1.6756 (1.2); 1.6702 (0.8); 1.6625 (2.5); 1.6548 (0.8); 1.6492 (1.3); 1.6416 (1.3); 1.6284 (0.7); 1.1947
(1.2); 1.1770 (2.6); 1.1592 (1.3); 0.7219 (1.3); 0.7112 (3.7); 0.7062 (3.8); 0.7010 (1.8); 0.6960 (1.9); 0.6903 (3.8); 0.6853 (3.6);
0.6752 (1.4); 0.3328 (1.6); 0.3226 (4.1); 0.3179 (4.6); 0.3094 (4.3); 0.3045 (4.3); 0.2940 (1.4); 0.0080 (2.2); −0.0002
(80.7); −0.0085 (2.5)
I-154: ¹H-NMR(400.6 MHz, CDCl3):
δ = 7.6210 (0.5); 7.6151 (0.9); 7.6003 (0.9); 7.5939 (0.6); 7.5791 (0.6); 7.5384 (0.8); 7.5181 (0.8); 7.2616 (8.8); 6.9425 (0.6);
6.9360 (0.7); 6.9335 (0.7); 6.9272 (0.6); 6.9207 (0.6); 6.9143 (0.7); 6.9117 (0.7); 6.9055 (0.6); 5.2595 (1.7); 5.2421 (1.7); 5.2248
(0.5); 3.7816 (16.0); 1.7040 (6.9); 1.6866 (6.8); 1.5791 (1.2); −0.0002 (11.4)
I-155: ¹H-NMR(400.6 MHz, CDCl3):
δ = 8.4972 (4.1); 8.4904 (4.1); 8.3133 (4.2); 7.6137 (1.5); 7.5982 (3.1); 7.5935 (3.9); 7.5915 (3.7); 7.5868 (2.0); 7.5765 (3.4);
7.5718 (3.8); 7.5695 (2.4); 7.5650 (1.9); 7.5568 (1.5); 7.2609 (41.7); 6.9416 (1.6); 6.9353 (1.9); 6.9325 (1.8); 6.9262 (1.7); 6.9198
(1.5); 6.9135 (1.7); 6.9106 (1.7); 6.9044 (1.4); 5.3191 (1.2); 5.3017 (4.8); 5.2842 (4.9); 5.2667 (1.2); 1.7482 (16.0); 1.7307 (15.9);
1.7053 (0.8); 1.6879 (0.7); 1.4321 (2.8); 1.2639 (0.5); 0.8819 (0.9); 0.0079 (1.6); −0.0002 (60.9); −0.0085 (1.7)
I-156: ¹H-NMR(400.6 MHz, CDCl3):
δ = 8.4527 (3.7); 8.4458 (3.8); 8.3335 (2.1); 8.3296 (3.8); 8.3259 (2.1); 8.0701 (1.7); 8.0582 (2.1); 7.7570 (1.9); 7.7526 (1.8);
7.7408 (4.6); 7.7371 (4.8); 7.7207 (0.6); 7.4341 (1.1); 7.4291 (1.3); 7.4278 (1.4); 7.4231 (1.1); 7.4112 (1.1); 7.4064 (1.3); 7.4049
(1.3); 7.4002 (1.1); 7.2625 (8.1); 7.1123 (1.2); 7.1073 (1.1); 7.1000 (1.2); 7.0954 (1.9); 7.0915 (1.2); 7.0845 (1.1); 7.0791 (1.0);
4.2622 (2.0); 4.2444 (6.0); 4.2266 (6.1); 4.2088 (2.0); 3.9607 (16.0); 1.6057 (1.0); 1.5980 (1.0); 1.5876 (5.9); 1.5772 (0.6); 1.5715
(1.0); 1.5639 (1.0); 1.5506 (0.5); 1.2992 (6.5); 1.2814 (13.0); 1.2636 (6.4); 0.7819 (1.0); 0.7705 (2.9); 0.7660 (2.8); 0.7614 (1.3);
0.7551 (1.4); 0.7496 (2.9); 0.7451 (2.7); 0.7343 (1.1); 0.4521 (1.2); 0.4406 (3.1); 0.4374 (3.4); 0.4276 (3.0); 0.4241 (3.2); 0.4122
(0.9); −0.0002 (11.5)
I-157: ¹H-NMR(400.6 MHz, CDCl3):
δ = 8.4655 (0.6); 8.4615 (0.6); 8.4536 (0.6); 8.4496 (0.6); 8.4282 (0.8); 8.4215 (0.8); 8.2772 (1.0); 8.1593 (0.7); 8.1549 (0.7);
8.1394 (0.7); 8.1351 (0.7); 7.5277 (0.5); 7.5234 (0.6); 7.5208 (0.6); 7.5165 (0.5); 7.5055 (0.5); 7.5012 (0.6); 7.4987 (0.6); 7.4943
(0.5); 7.4537 (0.5); 7.4417 (0.5); 7.2615 (9.2); 4.9309 (6.8); 4.3566 (1.7); 4.3482 (0.9); 4.3449 (1.7); 4.3412 (0.8); 4.3330 (1.8);
3.6233 (2.0); 3.6150 (0.9); 3.6113 (1.8); 3.6081 (0.9); 3.5996 (1.8); 3.3686 (16.0); 1.5885 (0.8); −0.0002 (13.0)
I-158: ¹H-NMR(400.6 MHz, CDCl3):
δ = 8.4475 (1.6); 8.4407 (1.6); 8.3270 (0.9); 8.3231 (1.6); 8.3193 (0.9); 7.5781 (0.5); 7.5630 (0.6); 7.5568 (0.8); 7.5420 (0.8);
7.5357 (0.6); 7.5207 (0.6); 7.4614 (0.7); 7.4571 (0.7); 7.4545 (0.7); 7.4502 (0.7); 7.4390 (0.7); 7.4346 (0.7); 7.4320 (0.7); 7.4277
(0.6); 7.2624 (10.6); 6.8486 (0.6); 6.8426 (0.6); 6.8392 (0.6); 6.8332 (0.6); 6.8269 (0.5); 6.8208 (0.6); 6.8175 (0.6); 6.8114 (0.5);
5.1992 (1.6); 5.1819 (1.6); 4.4787 (0.6); 4.4671 (0.8); 4.4509 (1.4); 4.4347 (1.5); 4.4189 (1.5); 4.4031 (0.8); 4.3911 (0.6); 3.6753
(16.0); 2.6788 (1.0); 2.6763 (1.0); 2.6627 (1.7); 2.6602 (2.1); 2.6466 (1.0); 2.6442 (1.0); 1.6245 (5.8); 1.6071 (5.8); 1.5801 (1.9);
1.5683 (0.6); 1.5552 (0.9); 1.5340 (0.6); 0.8395 (1.1); 0.8347 (1.4); 0.8268 (1.5); 0.8216 (1.2); 0.8137 (0.8); 0.7935 (0.7); 0.7849
(1.3); 0.7802 (0.9); 0.7724 (0.8); 0.7641 (1.0); 0.7596 (0.8); −0.0002 (13.4)
I-159: ¹H-NMR(400.6 MHz, d₆-DMSO):
δ = 13.0998 (2.2); 8.6970 (6.9); 8.6900 (7.0); 8.4349 (3.6); 8.4308 (6.6); 8.4272 (3.4); 8.2063 (1.5); 8.1909 (1.6); 8.1842 (3.0);
8.1688 (3.0); 8.1621 (1.7); 8.1467 (1.5); 7.9327 (2.5); 7.9283 (2.6); 7.9258 (2.6); 7.9214 (2.4); 7.9091 (2.7); 7.9047 (2.9); 7.9021
(2.5); 7.8977 (2.4); 7.4069 (1.6); 7.4004 (2.3); 7.3989 (2.3); 7.3923 (1.8); 7.3847 (1.6); 7.3782 (2.0); 7.3702 (1.5); 5.7570 (1.0);
5.1097 (1.2); 5.0924 (5.9); 5.0750 (6.0); 5.0576 (1.2); 3.3211 (12.7); 2.6752 (0.5); 2.6706 (0.7); 2.6660 (0.5); 2.5243 (1.8); 2.5197
(2.6); 2.5110 (37.5); 2.5064 (82.6); 2.5018 (116.3); 2.4972 (81.4); 2.4926 (36.8); 2.4785 (0.6); 2.4737 (0.6); 2.4690 (0.5); 2.3289
(0.7); 1.5898 (16.0); 1.5723 (15.9); 1.3556 (2.8); 1.2795 (0.6); 1.2576 (1.1); 1.2471 (3.2); 0.8750 (1.6); 0.8581 (6.5); 0.8404 (2.2);
0.0079 (3.8); 0.0064 (0.8); 0.0055 (0.9); 0.0046 (1.1); −0.0002 (140.8); −0.0051 (2.4); −0.0059 (2.0); −0.0068 (1.7); −0.0085
(4.4); −0.0115 (0.6); −0.0123 (0.5); −0.0131 (0.5); −0.0282 (0.6)
I-160: ¹H-NMR(400.6 MHz, CDCl3):
δ = 8.4706 (0.7); 8.4669 (0.8); 8.4588 (0.8); 8.4548 (0.8); 8.4305 (1.0); 8.4238 (1.1); 8.2813 (1.2); 8.1624 (0.8); 8.1582 (0.8);
8.1425 (0.9); 8.1384 (0.8); 7.5303 (0.6); 7.5259 (0.7); 7.5234 (0.7); 7.5191 (0.7); 7.5080 (0.7); 7.5037 (0.7); 7.5012 (0.7); 7.4968
(0.6); 7.4599 (0.6); 7.4583 (0.6); 7.4479 (0.6); 7.4463 (0.6); 7.4402 (0.6); 7.4385 (0.6); 7.4282 (0.6); 7.4266 (0.6); 7.2612 (15.6);
4.8830 (8.0); 4.4824 (1.9); 4.4663 (4.2); 4.4502 (2.0); 3.6934 (16.0); 2.6928 (1.9); 2.6767 (4.0); 2.6606 (1.9); 1.5936 (1.1); 0.0079
(0.6); −0.0002 (22.1); −0.0085 (0.6)
I-161: ¹H-NMR(400.6 MHz, CDCl3):
δ = 8.5184 (3.0); 8.5115 (3.1); 8.3842 (1.6); 8.3804 (3.0); 8.3765 (1.6); 7.8207 (3.3); 7.7993 (3.6); 7.5232 (1.2); 7.5189 (1.3);
7.5162 (1.3); 7.5119 (1.2); 7.5007 (1.3); 7.4964 (1.3); 7.4939 (1.3); 7.4896 (1.2); 7.4039 (2.5); 7.4024 (2.6); 7.3824 (2.3); 7.3810
(2.4); 7.2628 (8.0); 5.3003 (1.3); 4.9341 (14.4); 4.3228 (1.9); 4.3050 (6.0); 4.2872 (6.1); 4.2694 (2.0); 2.1741 (16.0); 1.6035 (0.6);
1.3338 (7.7); 1.3160 (15.9); 1.2982 (7.5); 0.0704 (1.2); −0.0002 (12.0)
I-162: ¹H-NMR(400.6 MHz, CDCl3):
δ = 8.4732 (1.4); 8.4694 (1.6); 8.4613 (1.5); 8.4574 (1.6); 8.4320 (2.3); 8.4252 (2.4); 8.2788 (2.6); 8.1632 (1.6); 8.1590 (1.6);
8.1433 (1.8); 8.1391 (1.7); 7.5279 (1.3); 7.5235 (1.4); 7.5210 (1.5); 7.5167 (1.3); 7.5057 (1.4); 7.5014 (1.5); 7.4989 (1.4); 7.4945
(1.3); 7.4624 (1.2); 7.4607 (1.2); 7.4504 (1.2); 7.4488 (1.2); 7.4426 (1.2); 7.4410 (1.2); 7.4307 (1.1); 7.4291 (1.1); 7.2613 (30.6);
4.8972 (16.0); 4.2274 (1.4); 4.2110 (1.4); 4.2005 (2.2); 4.1840 (2.2); 4.1112 (2.3); 4.0914 (2.3); 4.0842 (1.5); 4.0644 (1.5); 3.8533
(0.7); 3.8394 (0.8); 3.8323 (1.5); 3.8185 (1.6); 3.8121 (2.9); 3.7982 (1.2); 3.7943 (1.9); 3.7900 (2.1); 3.7721 (2.0); 3.7483 (1.2);
3.7294 (1.8); 3.7119 (1.4); 3.7097 (1.3); 3.6906 (0.9); 3.5478 (1.9); 3.5339 (2.0); 3.5256 (1.7); 3.5116 (1.7); 2.5981 (0.6); 2.5806
(0.8); 2.5634 (0.6); 2.0234 (0.6); 2.0109 (0.8); 2.0024 (0.5); 1.9978 (0.6); 1.9897 (0.8); 1.9780 (0.6); 1.9712 (0.5); 1.6502 (0.5);
1.6330 (0.7); 1.6182 (1.0); 1.5986 (1.0); 1.5819 (1.0); 1.5711 (4.3); 0.0080 (1.1); −0.0002 (44.6); −0.0085 (1.3)
I-163: ¹H-NMR(400.6 MHz, CDCl3):
δ = 8.4910 (1.9); 8.4846 (1.8); 8.3009 (2.2); 7.6340 (1.0); 7.6191 (1.1); 7.6125 (1.5); 7.5977 (1.5); 7.5921 (1.1); 7.5772 (1.0);
7.5445 (1.2); 7.5401 (1.3); 7.5378 (1.3); 7.5333 (1.1); 7.5225 (1.2); 7.5182 (1.3); 7.5159 (1.3); 7.5113 (1.1); 7.2613 (19.7); 6.9618
(1.1); 6.9555 (1.2); 6.9527 (1.2); 6.9463 (1.1); 6.9399 (1.0); 6.9337 (1.1); 6.9308 (1.1); 6.9245 (1.0); 4.9268 (16.0); 4.3021 (2.0);
4.2843 (6.1); 4.2665 (6.1); 4.2487 (2.0); 1.5663 (3.8); 1.3155 (7.2); 1.2977 (14.5); 1.2798 (7.0); 0.0079 (0.9); −0.0002
(25.9); −0.0085 (0.8)
I-164: ¹H-NMR(400.6 MHz, CDCl3):
δ = 7.5796 (0.6); 7.5646 (0.6); 7.5581 (0.8); 7.5431 (0.8); 7.5374 (0.6); 7.5224 (0.6); 7.4583 (0.5); 7.4558 (0.6); 7.4360 (0.5);
7.4334 (0.5); 7.2624 (7.2); 6.8754 (0.6); 6.8693 (0.7); 6.8661 (0.7); 6.8599 (0.7); 6.8537 (0.6); 6.8475 (0.6); 6.8443 (0.6); 6.8382
(0.6); 4.8887 (8.5); 3.7935 (16.0); 1.5873 (0.5); 1.5804 (1.6); 0.8131 (0.6); 0.8068 (3.3); 0.8057 (3.4); 0.8040 (2.7); 0.8012 (1.4);
0.7998 (1.5); 0.7980 (1.3); 0.7948 (2.2); 0.7915 (1.5); 0.7868 (1.6); 0.7835 (2.2); 0.7805 (0.9); 0.7783 (0.9); −0.0002 (10.3)
I-167: ¹H-NMR(400.6 MHz, CDCl3):
δ = 8.4118 (2.1); 8.4049 (2.1); 8.3147 (1.2); 8.3107 (2.1); 8.3069 (1.1); 8.2188 (1.0); 8.2171 (1.0); 8.2153 (1.0); 8.2071 (1.0);
8.2055 (1.1); 7.4804 (0.6); 7.4767 (0.7); 7.4598 (0.8); 7.4563 (1.3); 7.4533 (0.8); 7.4364 (0.8); 7.4326 (0.8); 7.4265 (0.8); 7.4222
(0.8); 7.4198 (0.8); 7.4154 (0.7); 7.4039 (0.7); 7.3996 (0.8); 7.3971 (0.8); 7.3927 (0.7); 7.2943 (0.8); 7.2853 (0.9); 7.2827 (0.9);
7.2737 (1.4); 7.2607 (20.2); 7.2531 (0.8); 4.9054 (9.2); 3.7871 (16.0); 1.6272 (0.9); 1.6103 (1.4); 1.5930 (1.0); 0.8070 (7.9);
0.7899 (9.3); 0.0081 (0.8); −0.0002 (30.0); −0.0085 (0.9)
I-171: ¹H-NMR(400.6 MHz, CDCl3):
δ = 8.4473 (1.3); 8.4406 (1.3); 8.3204 (1.6); 7.5755 (0.5); 7.5605 (0.6); 7.5543 (1.0); 7.5393 (1.0); 7.5332 (0.6); 7.5182 (0.6);
7.4588 (0.6); 7.4545 (0.7); 7.4521 (0.7); 7.4477 (0.6); 7.4364 (0.6); 7.4320 (0.7); 7.4296 (0.7); 7.4253 (0.6); 7.2613 (8.5); 6.8445
(0.6); 6.8384 (0.7); 6.8352 (0.7); 6.8291 (0.6); 6.8228 (0.6); 6.8168 (0.6); 6.8135 (0.6); 6.8074 (0.5); 5.2291 (1.6); 5.2117 (1.7);
3.7634 (16.0); 1.6381 (6.7); 1.6208 (6.7); 1.5834 (0.6); 1.5756 (0.9); 1.5671 (1.7); 1.5630 (1.5); 1.5491 (0.6); 1.5415 (0.6); 0.8407
(1.4); 0.8338 (1.5); 0.8278 (1.0); 0.8230 (1.0); 0.8201 (1.3); 0.8026 (0.5); 0.7989 (0.6); 0.7963 (0.6); 0.7897 (1.4); 0.7855 (1.0);
0.7784 (0.7); 0.7702 (1.3); 0.7642 (0.8); −0.0002 (11.6)
I-173: ¹H-NMR(400.6 MHz, CDCl3):
δ = 8.4937 (2.8); 8.4868 (2.9); 8.4178 (2.2); 8.1019 (1.2); 8.0998 (1.3); 8.0972 (1.4); 8.0951 (1.3); 8.0897 (1.3); 8.0876 (1.4);
8.0850 (1.4); 8.0829 (1.2); 7.8023 (1.1); 7.7976 (1.1); 7.7839 (1.3); 7.7816 (1.6); 7.7792 (1.3); 7.7769 (1.5); 7.7632 (1.6); 7.7585
(1.6); 7.6793 (1.7); 7.6770 (2.9); 7.6747 (1.7); 7.6586 (1.2); 7.6564 (2.0); 7.6539 (1.1); 7.5629 (0.7); 7.5616 (0.7); 7.5567 (1.0);
7.5519 (0.7); 7.5406 (0.7); 7.5392 (0.7); 7.5363 (0.8); 7.5344 (1.0); 7.5295 (0.7); 7.2612 (23.2); 7.1446 (1.4); 7.1420 (1.4); 7.1324
(1.4); 7.1298 (1.4); 7.1262 (1.4); 7.1237 (1.3); 7.1141 (1.3); 7.1115 (1.3); 4.9379 (14.6); 4.3299 (1.8); 4.3121 (5.7); 4.2943 (5.8);
4.2765 (1.9); 1.3376 (7.7); 1.3198 (16.0); 1.3020 (7.4); 0.0081 (1.0); −0.0002 (36.7); −0.0085 (1.0)
I-174: ¹H-NMR(400.6 MHz, CDCl3):
δ = 8.4107 (3.1); 8.4038 (3.1); 8.3149 (1.8); 8.3110 (3.2); 8.3072 (1.7); 8.2183 (1.3); 8.2165 (1.6); 8.2147 (1.6); 8.2130 (1.4);
8.2066 (1.4); 8.2048 (1.6); 8.2031 (1.5); 8.2013 (1.3); 7.4789 (1.1); 7.4752 (1.1); 7.4583 (1.5); 7.4550 (2.0); 7.4517 (1.2); 7.4349
(1.4); 7.4311 (1.4); 7.4265 (1.2); 7.4222 (1.2); 7.4196 (1.2); 7.4153 (1.1); 7.4039 (1.2); 7.3995 (1.3); 7.3970 (1.2); 7.3926 (1.1);
7.2916 (1.4); 7.2826 (1.5); 7.2800 (1.4); 7.2710 (2.5); 7.2619 (15.8); 7.2504 (1.1); 4.8876 (15.8); 4.2761 (2.0); 4.2583 (6.2);
4.2405 (6.2); 4.2227 (2.0); 2.0451 (0.6); 1.6311 (0.6); 1.6293 (0.6); 1.6249 (0.8); 1.6173 (0.6); 1.6104 (2.0); 1.6028 (0.6); 1.5971
(0.7); 1.5950 (0.7); 1.5909 (0.9); 1.5760 (1.2); 1.3018 (7.8); 1.2840 (16.0); 1.2773 (0.6); 1.2661 (7.9); 1.2595 (0.8); 0.8819 (1.0);
0.8218 (1.2); 0.8170 (4.0); 0.8147 (3.6); 0.8123 (3.7); 0.8097 (4.9); 0.8035 (4.1); 0.7995 (2.3); 0.7933 (2.4); 0.7896 (3.6); 0.7866
(1.6); 0.7841 (1.5); 0.0079 (0.6); −0.0002 (22.2); −0.0085 (0.6)
I-175: ¹H-NMR(400.0 MHz, d₆-DMSO):
δ = 12.9139 (2.1); 8.5774 (5.4); 8.5704 (5.5); 8.3111 (2.9); 8.3069 (5.5); 8.3030 (2.8); 8.2048 (2.8); 8.1934 (2.9); 7.9328 (1.6);
7.9292 (1.6); 7.9119 (1.8); 7.9079 (2.8); 7.9039 (1.8); 7.8866 (1.8); 7.8830 (1.7); 7.7473 (1.7); 7.7429 (1.8); 7.7404 (1.9); 7.7360
(1.6); 7.7231 (1.8); 7.7188 (2.0); 7.7163 (1.8); 7.7119 (1.6); 7.5295 (1.8); 7.5202 (2.1); 7.5180 (2.0); 7.5086 (3.2); 7.4994 (1.6);
7.4971 (1.7); 7.4878 (1.4); 5.7561 (0.8); 4.7741 (16.0); 3.6012 (1.2); 3.3192 (13.4); 2.5241 (1.2); 2.5194 (1.7); 2.5106 (27.5);
2.5061 (61.0); 2.5015 (85.5); 2.4969 (60.6); 2.4924 (27.3); 1.7596 (1.4); 1.7244 (0.6); 1.7114 (1.3); 1.7034 (1.3); 1.6985 (0.8);
1.6903 (2.7); 1.6825 (0.8); 1.6772 (1.4); 1.6693 (1.4); 1.6563 (0.7); 0.7655 (1.3); 0.7546 (3.1); 0.7490 (4.1); 0.7446 (1.8); 0.7400
(2.2); 0.7336 (3.2); 0.7278 (3.8); 0.7190 (1.8); 0.6395 (1.8); 0.6304 (4.0); 0.6255 (4.3); 0.6175 (4.0); 0.6122 (3.5); 0.6011 (1.2);
0.0080 (1.5); −0.0002 (52.5); −0.0085 (1.5)
I-177: ¹H-NMR(400.6 MHz, CDCl3):
δ = 8.4451 (3.1); 8.4382 (3.2); 8.3220 (1.7); 8.3182 (3.2); 8.3144 (1.8); 7.5708 (1.0); 7.5557 (1.1); 7.5495 (1.6); 7.5345 (1.6);
7.5284 (1.2); 7.5134 (1.1); 7.4578 (1.3); 7.4534 (1.4); 7.4509 (1.4); 7.4465 (1.3); 7.4353 (1.3); 7.4310 (1.4); 7.4284 (1.3); 7.4241
(1.2); 7.2621 (14.6); 6.8424 (1.1); 6.8364 (1.3); 6.8330 (1.2); 6.8269 (1.2); 6.8207 (1.1); 6.8146 (1.1); 6.8113 (1.1); 6.8052 (1.0);
5.2303 (0.8); 5.2129 (3.2); 5.1956 (3.2); 5.1783 (0.9); 4.2463 (2.1); 4.2285 (6.8); 4.2108 (6.9); 4.1930 (2.2); 1.6346 (12.3); 1.6173
(12.4); 1.5979 (0.9); 1.5847 (1.1); 1.5768 (1.1); 1.5701 (0.6); 1.5639 (2.0); 1.5556 (0.7); 1.5503 (1.0); 1.5426 (1.2); 1.5294 (0.6);
1.2813 (7.7); 1.2636 (16.0); 1.2458 (7.5); 0.8693 (0.7); 0.8632 (0.7); 0.8562 (2.3); 0.8519 (2.4); 0.8436 (3.1); 0.8384 (2.3); 0.8303
(1.8); 0.8086 (0.6); 0.7970 (1.6); 0.7882 (2.7); 0.7845 (1.4); 0.7829 (1.3); 0.7762 (2.0); 0.7712 (1.0); 0.7672 (2.2); 0.7629 (1.6);
0.7577 (0.8); 0.7504 (0.8); 0.0080 (0.5); −0.0002 (20.1); −0.0084 (0.6)
I-178: ¹H-NMR(400.6 MHz, CDCl3):
δ = 8.4669 (1.3); 8.4601 (1.3); 8.2901 (1.2); 8.2034 (0.7); 8.2017 (0.8); 8.1998 (0.8); 8.1981 (0.8); 8.1918 (0.7); 8.1901 (0.9);
8.1882 (0.8); 8.1866 (0.8); 7.5241 (0.6); 7.5204 (0.6); 7.5033 (0.8); 7.5013 (0.8); 7.4997 (0.9); 7.4977 (0.7); 7.4806 (0.8); 7.4768
(1.1); 7.4704 (0.5); 7.3655 (0.7); 7.3564 (0.8); 7.3539 (0.8); 7.3447 (1.3); 7.3356 (0.6); 7.3331 (0.6); 7.3240 (0.6); 7.2621 (10.9);
5.2838 (1.4); 5.2664 (1.4); 3.7680 (16.0); 3.7605 (0.6); 1.6876 (6.2); 1.6702 (6.2); 1.5809 (2.3); −0.0002 (12.0)
I-179: ¹H-NMR(400.6 MHz, CDCl3):
δ = 8.4013 (1.8); 8.3944 (1.8); 8.2682 (1.0); 8.2645 (1.8); 8.2609 (1.0); 8.2073 (0.7); 8.2058 (0.7); 8.2028 (0.7); 8.2014 (0.7);
8.1954 (0.7); 8.1940 (0.7); 8.1910 (0.7); 8.1895 (0.7); 7.6495 (0.7); 7.6476 (0.7); 7.6450 (0.7); 7.6432 (0.7); 7.6304 (0.7); 7.6286
(0.8); 7.6259 (0.8); 7.6241 (0.7); 7.5154 (0.8); 7.5110 (0.8); 7.5085 (0.8); 7.5041 (0.8); 7.4928 (0.8); 7.4884 (0.9); 7.4858 (0.8);
7.4814 (0.8); 7.2626 (8.0); 7.2427 (1.0); 7.2308 (1.0); 7.2303 (1.0); 7.2236 (0.9); 7.2118 (0.9); 5.3001 (2.1); 4.9162 (8.1); 3.7893
(16.0); 2.2133 (7.8); 1.6020 (0.9); 0.0699 (0.8); −0.0002 (10.9)
I-180: ¹H-NMR(400.6 MHz, CDCl3):
δ = 8.4709 (2.1); 8.4647 (2.1); 8.3341 (2.6); 7.5761 (1.5); 7.5611 (1.6); 7.5548 (2.3); 7.5401 (2.3); 7.5338 (1.6); 7.5190 (1.6);
7.4893 (1.7); 7.4850 (1.8); 7.4825 (1.8); 7.4782 (1.6); 7.4671 (1.7); 7.4628 (1.9); 7.4603 (1.7); 7.4560 (1.5); 7.2681 (0.7); 7.2674
(0.7); 7.2614 (104.2); 6.9978 (0.5); 6.8603 (1.6); 6.8542 (1.8); 6.8510 (1.8); 6.8448 (1.7); 6.8385 (1.5); 6.8324 (1.6); 6.8292 (1.6);
6.8231 (1.4); 5.3002 (1.2); 5.2980 (1.2); 5.2806 (5.0); 5.2632 (5.2); 5.2458 (1.2); 1.6875 (16.0); 1.6701 (15.9); 1.6553 (0.5);
1.5925 (0.7); 1.5779 (1.5); 1.5737 (1.0); 1.5719 (0.8); 1.5628 (0.9); 1.5589 (2.3); 1.5507 (1.0); 1.5428 (1.2); 1.5400 (1.1); 1.5374
(0.9); 1.5239 (0.9); 1.4322 (2.7); 1.2412 (0.6); 0.8306 (0.7); 0.8203 (0.7); 0.8151 (2.0); 0.8123 (2.4); 0.8070 (1.8); 0.7996 (5.6);
0.7931 (8.0); 0.7858 (3.3); 0.7797 (3.0); 0.7725 (8.0); 0.7576 (1.1); 0.0080 (1.5); −0.0002 (60.8); −0.0049 (0.8); −0.0057
(0.7); −0.0085 (1.7)
I-181: ¹H-NMR(400.6 MHz, CDCl3):
δ = 8.3986 (1.7); 8.3918 (1.8); 8.2486 (2.0); 8.2142 (1.0); 8.2128 (1.1); 8.2098 (1.2); 8.2084 (1.1); 8.2023 (1.1); 8.2009 (1.2);
8.1979 (1.2); 8.1965 (1.1); 7.6559 (1.0); 7.6541 (1.1); 7.6515 (1.2); 7.6497 (1.0); 7.6369 (1.1); 7.6350 (1.3); 7.6324 (1.3); 7.6306
(1.1); 7.4991 (1.1); 7.4947 (1.1); 7.4922 (1.2); 7.4878 (1.1); 7.4764 (1.1); 7.4720 (1.2); 7.4695 (1.2); 7.4651 (1.1); 7.2624 (13.8);
7.2484 (1.5); 7.2364 (1.5); 7.2294 (1.4); 7.2175 (1.4); 5.3000 (8.8); 4.8995 (14.4); 4.2816 (1.8); 4.2637 (5.8); 4.2459 (5.9); 4.2281
(1.9); 2.2205 (12.7); 1.5888 (2.7); 1.3043 (7.6); 1.2865 (16.0); 1.2687 (7.5); 0.0699 (0.7); 0.0022 (0.5); −0.0002 (17.1); −0.0027
(0.9); −0.0085 (0.5)
I-182: ¹H-NMR(400.6 MHz, d₆-DMSO):
δ = 13.0901 (0.8); 8.6015 (4.4); 8.5945 (4.5); 8.3090 (2.4); 8.3048 (4.5); 8.3008 (2.2); 8.1755 (1.6); 8.1725 (1.8); 8.1711 (1.7);
8.1651 (1.6); 8.1637 (1.8); 8.1607 (1.7); 8.1594 (1.7); 7.8908 (1.4); 7.8891 (1.6); 7.8865 (1.7); 7.8848 (1.4); 7.8718 (1.6); 7.8700
(1.8); 7.8675 (1.7); 7.8657 (1.5); 7.7387 (1.5); 7.7344 (1.6); 7.7318 (1.6); 7.7274 (1.4); 7.7149 (1.6); 7.7106 (1.8); 7.7080 (1.6);
7.7036 (1.4); 7.4044 (2.3); 7.3926 (2.2); 7.3855 (2.2); 7.3736 (2.1); 5.7565 (14.3); 4.8556 (12.5); 3.3217 (2.1); 2.5247 (0.8);
2.5199 (1.2); 2.5111 (16.1); 2.5066 (35.6); 2.5020 (49.7); 2.4974 (35.0); 2.4928 (15.4); 2.2502 (16.0); 1.3560 (0.7); 0.0080
(1.0); −0.0002 (35.3); −0.0085 (1.0)
I-388: ¹H-NMR(400.6 MHz, CDCl3):
δ = 8.4847 (1.5); 8.4781 (1.5); 8.3022 (1.8); 7.6211 (1.0); 7.6062 (1.1); 7.6008 (1.2); 7.5994 (1.3); 7.5859 (1.3); 7.5845 (1.3);
7.5791 (1.1); 7.5642 (1.1); 7.5486 (1.2); 7.5443 (1.2); 7.5417 (1.2); 7.5373 (1.1); 7.5266 (1.2); 7.5222 (1.3); 7.5196 (1.4); 7.5153
(1.1); 7.2609 (30.6); 6.9326 (1.1); 6.9264 (1.2); 6.9234 (1.2); 6.9171 (1.2); 6.9108 (1.0); 6.9046 (1.1); 6.9015 (1.1); 6.8953 (1.0);
5.2474 (0.8); 5.2300 (3.1); 5.2126 (3.1); 5.1952 (0.8); 4.2655 (2.1); 4.2477 (6.8); 4.2299 (6.8); 4.2121 (2.2); 1.6977 (11.8); 1.6803
(11.7); 1.5681 (0.6); 1.2872 (7.6); 1.2694 (16.0); 1.2516 (7.4); 0.0080 (1.2); −0.0002 (41.4); −0.0085 (1.1)
I-183: ¹H-NMR(400.6 MHz, CDCl3):
δ = 8.4912 (3.1); 8.4843 (3.1); 8.3422 (1.6); 8.3383 (3.0); 8.3345 (1.6); 8.1153 (1.1); 8.1131 (1.2); 8.1109 (1.4); 8.1086 (1.2);
8.1032 (1.2); 8.1008 (1.3); 8.0990 (1.4); 8.0965 (1.2); 7.8395 (0.6); 7.8349 (0.6); 7.8223 (0.5); 7.8188 (1.8); 7.8143 (1.7); 7.8017
(2.1); 7.7973 (2.2); 7.7955 (2.2); 7.7931 (2.3); 7.7916 (2.5); 7.7890 (2.2); 7.7748 (0.7); 7.7725 (0.8); 7.5049 (1.2); 7.5005 (1.2);
7.4980 (1.2); 7.4936 (1.2); 7.4824 (1.2); 7.4781 (1.2); 7.4755 (1.2); 7.4712 (1.1); 7.2617 (13.5); 7.1931 (1.2); 7.1892 (1.2); 7.1810
(1.2); 7.1763 (1.9); 7.1720 (1.2); 7.1640 (1.2); 7.1599 (1.2); 5.3000 (5.8); 4.2637 (1.9); 4.2459 (6.1); 4.2281 (6.2); 4.2103 (2.0);
3.9279 (16.0); 1.7277 (1.0); 1.2988 (7.3); 1.2810 (14.6); 1.2632 (7.1); 1.2563 (1.4); 0.0080 (0.6); −0.0002 (20.5); −0.0085 (0.6)
I-184: ¹H-NMR(400.6 MHz, CDCl3):
δ = 8.4822 (3.3); 8.4753 (3.4); 8.3671 (1.7); 8.3632 (3.3); 8.3594 (1.7); 8.0506 (1.2); 8.0486 (1.4); 8.0460 (1.4); 8.0439 (1.4);
8.0385 (1.3); 8.0364 (1.4); 8.0338 (1.4); 8.0317 (1.3); 7.7734 (1.1); 7.7687 (1.1); 7.7550 (1.2); 7.7528 (1.6); 7.7503 (1.3); 7.7481
(1.6); 7.7344 (1.6); 7.7296 (1.6); 7.6493 (1.7); 7.6470 (3.1); 7.6447 (1.8); 7.6287 (1.3); 7.6263 (2.2); 7.6240 (1.2); 7.5183 (1.3);
7.5139 (1.3); 7.5114 (1.4); 7.5070 (1.2); 7.4957 (1.3); 7.4914 (1.4); 7.4888 (1.3); 7.4844 (1.2); 7.2614 (19.3); 7.1106 (1.4); 7.1080
(1.4); 7.0984 (1.4); 7.0958 (1.4); 7.0923 (1.4); 7.0896 (1.4); 7.0801 (1.4); 7.0775 (1.3); 5.3001 (1.8); 5.2611 (0.8); 5.2437 (3.1);
5.2263 (3.1); 5.2089 (0.9); 4.2958 (1.0); 4.2892 (1.0); 4.2872 (0.6); 4.2780 (3.1); 4.2714 (3.1); 4.2602 (3.2); 4.2537 (3.1); 4.2424
(1.1); 4.2360 (1.0); 1.7188 (11.8); 1.7014 (11.7); 1.5695 (2.4); 1.3058 (7.6); 1.2880 (16.0); 1.2702 (7.5); 1.2545 (0.7); 0.0080
(0.8); −0.0002 (26.2); −0.0085 (0.8)
I-185: ¹H-NMR(400.6 MHz, CDCl3):
δ = 8.4970 (1.9); 8.4901 (1.9); 8.3770 (1.0); 8.3731 (1.9); 8.3694 (1.0); 8.0749 (0.8); 8.0728 (0.8); 8.0702 (0.9); 8.0681 (0.8);
8.0627 (0.8); 8.0606 (0.9); 8.0581 (0.9); 8.0560 (0.8); 7.8130 (0.6); 7.8083 (0.6); 7.7947 (0.7); 7.7924 (1.0); 7.7901 (0.7); 7.7877
(1.0); 7.7741 (1.1); 7.7694 (1.1); 7.7239 (1.1); 7.7214 (1.9); 7.7190 (1.2); 7.7032 (0.7); 7.7009 (1.1); 7.6984 (0.7); 7.5219 (0.8);
7.5176 (0.8); 7.5151 (0.8); 7.5107 (0.8); 7.4995 (0.8); 7.4951 (0.8); 7.4926 (0.8); 7.4882 (0.8); 7.2615 (17.3); 7.1488 (0.9); 7.1460
(0.9); 7.1366 (0.8); 7.1338 (0.9); 7.1306 (0.9); 7.1278 (0.8); 7.1184 (0.9); 7.1156 (0.8); 5.3970 (1.4); 5.3799 (1.5); 5.3002 (1.3);
4.1468 (1.6); 4.1332 (1.5); 4.1177 (1.5); 4.1045 (1.5); 3.7705 (16.0); 1.7442 (7.0); 1.7272 (7.0); 1.5700 (2.7); 0.0079 (0.7); −0.0002
(23.3); −0.0085 (0.6)
I-186: ¹H-NMR(400.6 MHz, d₆-DMSO):
δ = 12.9870 (0.5); 8.6258 (3.1); 8.6188 (3.1); 8.5955 (1.1); 8.5916 (1.3); 8.5834 (1.2); 8.5795 (1.3); 8.4593 (1.3); 8.4552 (1.3);
8.4393 (1.4); 8.4352 (1.3); 8.2746 (1.6); 8.2705 (3.0); 8.2668 (1.5); 7.7471 (1.1); 7.7427 (1.1); 7.7402 (1.1); 7.7358 (1.0); 7.7237
(1.1); 7.7193 (1.2); 7.7168 (1.1); 7.7124 (1.1); 7.7066 (0.9); 7.6944 (0.9); 7.6880 (0.9); 7.6760 (0.8); 5.7571 (16.0); 4.8018 (7.9);
3.3236 (3.5); 2.5252 (0.7); 2.5205 (1.0); 2.5117 (13.6); 2.5072 (29.4); 2.5026 (41.0); 2.4980 (27.9); 2.4934 (12.1); 0.0080
(1.1); −0.0002 (37.3); −0.0085 (1.0)
I-187: ¹H-NMR(400.6 MHz, CDCl3):
δ = 8.4985 (2.0); 8.4916 (2.0); 8.3711 (1.1); 8.3674 (2.0); 8.3637 (1.0); 8.0643 (0.8); 8.0623 (0.9); 8.0597 (0.9); 8.0576 (0.8);
8.0522 (0.8); 8.0501 (0.9); 8.0476 (0.9); 8.0455 (0.8); 7.8088 (0.6); 7.8041 (0.6); 7.7905 (0.7); 7.7881 (1.1); 7.7859 (0.7); 7.7835
(1.0); 7.7699 (1.1); 7.7652 (1.0); 7.7259 (1.2); 7.7234 (1.9); 7.7210 (1.2); 7.7052 (0.7); 7.7029 (1.1); 7.7003 (0.6); 7.5191 (0.8);
7.5147 (0.8); 7.5121 (0.8); 7.5078 (0.7); 7.4966 (0.8); 7.4923 (0.9); 7.4897 (0.8); 7.4854 (0.7); 7.2622 (10.3); 7.1427 (0.9); 7.1399
(0.9); 7.1306 (0.8); 7.1278 (0.9); 7.1245 (0.9); 7.1217 (0.8); 7.1124 (0.9); 7.1096 (0.8); 5.3979 (1.4); 5.3809 (1.5); 5.3001 (3.2);
4.1484 (1.6); 4.1348 (1.5); 4.1189 (1.4); 4.1058 (1.5); 3.7732 (16.0); 1.7469 (7.0); 1.7299 (6.9); 1.5880 (0.8); 1.2594 (0.5); −0.0002
(14.4); −0.0027 (0.6)
I-188: ¹H-NMR(400.6 MHz, CDCl3):
δ = 8.4848 (3.5); 8.4779 (3.6); 8.3678 (1.6); 8.3639 (3.1); 8.3601 (1.8); 8.0415 (0.7); 8.0394 (0.8); 8.0368 (1.4); 8.0348 (1.5);
8.0321 (1.0); 8.0297 (1.4); 8.0274 (1.0); 8.0246 (1.5); 8.0226 (1.5); 8.0200 (0.9); 8.0179 (0.8); 7.7866 (0.6); 7.7819 (0.6); 7.7784
(0.6); 7.7737 (0.6); 7.7682 (0.7); 7.7659 (0.9); 7.7635 (0.8); 7.7606 (1.1); 7.7578 (1.0); 7.7553 (0.8); 7.7530 (0.9); 7.7476 (0.9);
7.7429 (0.9); 7.7394 (0.9); 7.7347 (0.8); 7.6656 (0.9); 7.6633 (1.7); 7.6608 (1.8); 7.6583 (1.7); 7.6561 (1.0); 7.6450 (0.7); 7.6426
(1.2); 7.6401 (1.3); 7.6377 (1.2); 7.6354 (0.7); 7.5215 (1.0); 7.5172 (1.1); 7.5146 (1.1); 7.5103 (1.0); 7.4990 (1.0); 7.4947 (1.2);
7.4922 (1.1); 7.4878 (1.0); 7.2618 (18.7); 7.1086 (0.9); 7.1063 (1.3); 7.1047 (1.0); 7.0942 (1.3); 7.0925 (1.1); 7.0904 (1.0); 7.0881
(1.2); 7.0864 (0.9); 7.0782 (0.9); 7.0759 (1.1); 7.0742 (0.9); 5.3001 (4.5); 5.2597 (1.6); 5.2516 (1.6); 5.2423 (1.6); 5.2342 (1.6);
4.4130 (0.9); 4.3951 (0.9); 4.3859 (1.3); 4.3680 (1.4); 4.3357 (2.3); 4.3338 (2.4); 4.3186 (3.7); 4.2795 (1.3); 4.2654 (1.4); 4.2525
(0.9); 4.2383 (1.0); 3.8017 (1.1); 3.7258 (2.1); 3.6588 (16.0); 3.6508 (16.0); 2.8363 (0.6); 2.8325 (0.8); 2.8227 (0.8); 2.8184 (0.8);
2.8147 (0.6); 2.8060 (0.8); 2.8005 (0.5); 1.7096 (6.6); 1.7075 (6.8); 1.6922 (6.6); 1.6901 (6.7); 1.5742 (1.9); 1.1979 (1.1); 1.1881
(6.5); 1.1797 (1.3); 1.1702 (12.9); 1.1522 (6.4); 0.0080 (0.7); −0.0002 (24.5); −0.0085 (0.6)
I-189: ¹H-NMR(400.6 MHz, CDCl3):
δ = 8.4830 (1.8); 8.4761 (1.8); 8.3681 (1.0); 8.3642 (1.8); 8.3604 (1.0); 8.0546 (0.7); 8.0526 (0.7); 8.0499 (0.8); 8.0479 (0.7);
8.0425 (0.7); 8.0405 (0.8); 8.0378 (0.8); 8.0358 (0.7); 7.7809 (0.6); 7.7762 (0.6); 7.7624 (0.7); 7.7603 (0.8); 7.7578 (0.7); 7.7556
(0.8); 7.7418 (0.8); 7.7371 (0.8); 7.6404 (0.9); 7.6381 (1.6); 7.6358 (1.0); 7.6197 (0.7); 7.6174 (1.2); 7.6151 (0.7); 7.5179 (0.7);
7.5136 (0.7); 7.5110 (0.7); 7.5067 (0.7); 7.4953 (0.7); 7.4910 (0.7); 7.4884 (0.7); 7.4841 (0.6); 7.2620 (6.3); 7.1162 (0.7); 7.1136
(0.8); 7.1041 (0.7); 7.1015 (0.8); 7.0978 (0.8); 7.0953 (0.7); 7.0857 (0.7); 7.0831 (0.7); 5.2786 (1.5); 5.2612 (1.5); 3.8030 (16.0);
1.7236 (6.3); 1.7061 (6.2); 1.5995 (0.7); 1.2643 (0.7); 0.8818 (1.5); 0.8641 (0.6); −0.0002 (8.3)
I-190: ¹H-NMR(400.6 MHz, CDCl3):
δ = 8.5005 (3.3); 8.4936 (3.5); 8.3773 (1.0); 8.3734 (1.9); 8.3702 (1.8); 8.3668 (1.9); 8.3631 (1.1); 8.0649 (0.8); 8.0630 (1.4);
8.0606 (1.4); 8.0584 (1.5); 8.0564 (0.9); 8.0528 (0.9); 8.0509 (1.5); 8.0486 (1.5); 8.0462 (1.5); 8.0443 (0.8); 7.8091 (0.6); 7.8052
(0.8); 7.8008 (0.5); 7.7907 (0.7); 7.7882 (1.2); 7.7846 (1.4); 7.7801 (0.9); 7.7702 (1.0); 7.7664 (1.3); 7.7620 (1.0); 7.7246 (1.1);
7.7221 (1.8); 7.7200 (1.9); 7.7179 (1.8); 7.7154 (1.1); 7.7040 (0.7); 7.7016 (1.0); 7.6994 (1.1); 7.6973 (1.1); 7.6947 (0.6); 7.5252
(0.7); 7.5203 (1.3); 7.5184 (0.9); 7.5156 (0.9); 7.5137 (1.2); 7.5088 (0.7); 7.5028 (0.8); 7.4981 (1.2); 7.4959 (0.9); 7.4932 (0.9);
7.4913 (1.2); 7.4864 (0.7); 7.2616 (26.8); 7.1435 (1.0); 7.1407 (1.2); 7.1393 (1.2); 7.1364 (1.2); 7.1314 (1.2); 7.1285 (1.3); 7.1271
(1.3); 7.1252 (1.6); 7.1225 (1.4); 7.1212 (1.4); 7.1183 (1.2); 7.1132 (1.0); 7.1102 (1.1); 7.1091 (1.1); 7.1061 (1.0); 5.3563 (0.5);
5.3518 (1.6); 5.3394 (1.6); 5.3348 (1.6); 5.3224 (1.6); 5.3002 (4.0); 4.6575 (1.1); 4.6397 (1.7); 4.6218 (1.1); 3.7862 (16.0); 3.7272
(15.5); 1.7332 (7.9); 1.7321 (7.6); 1.7161 (7.9); 1.7151 (7.6); 1.5735 (2.2); 1.4986 (6.2); 1.4808 (6.1); 1.4493 (6.3); 1.4314 (6.2);
1.2596 (0.8); 0.8819 (1.2); 0.0080 (1.1); −0.0002 (36.6); −0.0085 (1.0)
I-191: ¹H-NMR(400.6 MHz, CDCl3):
δ = 7.6281 (0.5); 7.6133 (0.6); 7.6078 (0.7); 7.6064 (0.7); 7.5929 (0.7); 7.5916 (0.7); 7.5861 (0.6); 7.5712 (0.6); 7.5453 (0.5);
7.5429 (0.6); 7.5233 (0.6); 7.5207 (0.6); 7.2619 (8.2); 6.9365 (0.6); 6.9302 (0.6); 6.9272 (0.6); 6.9210 (0.6); 6.9146 (0.5); 6.9084
(0.6); 6.9054 (0.6); 6.8991 (0.5); 5.2545 (1.5); 5.2371 (1.6); 3.7798 (16.0); 1.7026 (6.4); 1.6852 (6.3); 1.5724 (1.2); −0.0002 (10.9)
I-192: ¹H-NMR(400.6 MHz, CDCl3):
δ = 8.0750 (0.6); 8.0730 (0.6); 8.0704 (0.7); 8.0683 (0.6); 8.0629 (0.6); 8.0609 (0.7); 8.0582 (0.7); 8.0562 (0.6); 7.7959 (0.5);
7.7911 (0.5); 7.7774 (0.6); 7.7752 (0.8); 7.7727 (0.6); 7.7705 (0.8); 7.7568 (0.8); 7.7521 (0.7); 7.6713 (0.8); 7.6689 (1.4); 7.6667
(0.8); 7.6506 (0.6); 7.6483 (1.0); 7.6460 (0.6); 7.5221 (0.5); 7.2622 (7.1); 7.1330 (0.6); 7.1304 (0.6); 7.1208 (0.6); 7.1182 (0.7);
7.1146 (0.7); 7.1120 (0.6); 7.1025 (0.6); 7.0999 (0.6); 4.9554 (6.7); 4.4232 (0.6); 4.4053 (0.6); 4.3961 (1.3); 4.3782 (1.4); 4.3527
(1.3); 4.3386 (1.4); 4.3256 (0.6); 4.3115 (0.6); 3.6748 (16.0); 2.8460 (0.7); 2.8319 (0.7); 1.2067 (6.4); 1.1887 (6.3); −0.0002 (10.4)
I-193: ¹H-NMR(400.6 MHz, CDCl3):
δ = 8.4917 (2.5); 8.4848 (2.5); 8.3113 (1.6); 8.3078 (2.6); 8.3045 (1.6); 7.6268 (1.0); 7.6120 (1.0); 7.6067 (1.2); 7.6051 (1.3);
7.5918 (1.2); 7.5903 (1.3); 7.5850 (1.1); 7.5701 (1.0); 7.5528 (1.2); 7.5484 (1.3); 7.5459 (1.3); 7.5415 (1.2); 7.5308 (1.3); 7.5264
(1.4); 7.5240 (1.3); 7.5196 (1.2); 7.2622 (14.0); 6.9565 (1.1); 6.9502 (1.2); 6.9473 (1.2); 6.9410 (1.1); 6.9346 (1.0); 6.9284 (1.1);
6.9254 (1.1); 6.9192 (1.0); 4.9252 (15.6); 4.2998 (1.9); 4.2820 (6.1); 4.2642 (6.2); 4.2464 (2.0); 1.5690 (5.0); 1.3141 (7.7); 1.2963
(16.0); 1.2785 (7.5); 0.0080 (0.6); −0.0002 (18.9); −0.0084 (0.6)
I-194: ¹H-NMR(400.6 MHz, CDCl3):
δ = 8.4912 (1.0); 8.4848 (1.0); 8.3661 (1.2); 8.0633 (0.7); 8.0613 (0.8); 8.0587 (0.8); 8.0567 (0.8); 8.0512 (0.8); 8.0492 (0.8);
8.0466 (0.8); 8.0446 (0.7); 7.7908 (0.6); 7.7861 (0.6); 7.7724 (0.7); 7.7702 (0.9); 7.7677 (0.8); 7.7655 (0.8); 7.7518 (0.9); 7.7471
(0.8); 7.6724 (1.0); 7.6704 (1.6); 7.6683 (1.0); 7.6518 (0.7); 7.6498 (1.1); 7.6476 (0.6); 7.5261 (0.6); 7.5218 (0.7); 7.5193 (0.7);
7.5150 (0.6); 7.5037 (0.6); 7.4993 (0.7); 7.4968 (0.7); 7.4925 (0.6); 7.2621 (7.3); 7.1262 (0.8); 7.1237 (0.8); 7.1141 (0.8); 7.1115
(0.8); 7.1079 (0.8); 7.1053 (0.7); 7.0958 (0.7); 7.0932 (0.7); 5.3002 (1.5); 4.9543 (7.8); 4.4220 (0.7); 4.4041 (0.7); 4.3949 (1.4);
4.3770 (1.5); 4.3509 (1.4); 4.3369 (1.5); 4.3238 (0.7); 4.3098 (0.7); 3.7258 (2.2); 3.6749 (16.0); 2.8480 (0.6); 2.8443 (0.8); 2.8301
(0.8); 2.8264 (0.5); 1.2054 (6.8); 1.1979 (1.3); 1.1875 (6.7); 1.1798 (1.2); −0.0002 (10.7)
I-195: ¹H-NMR(400.6 MHz, CDCl3):
δ = 8.4909 (1.2); 8.4841 (1.2); 8.3691 (0.8); 8.3655 (1.3); 8.0634 (0.6); 8.0614 (0.7); 8.0588 (0.8); 8.0567 (0.7); 8.0513 (0.7);
8.0493 (0.8); 8.0466 (0.8); 8.0446 (0.7); 7.7907 (0.6); 7.7860 (0.6); 7.7723 (0.6); 7.7701 (0.8); 7.7677 (0.7); 7.7654 (0.8); 7.7517
(0.8); 7.7470 (0.8); 7.6725 (0.8); 7.6702 (1.6); 7.6680 (0.9); 7.6519 (0.6); 7.6496 (1.1); 7.6474 (0.6); 7.5262 (0.6); 7.5218 (0.7);
7.5193 (0.7); 7.5150 (0.6); 7.5037 (0.6); 7.4994 (0.7); 7.4968 (0.7); 7.4925 (0.6); 7.2614 (12.1); 7.1263 (0.7); 7.1237 (0.7); 7.1141
(0.7); 7.1115 (0.8); 7.1079 (0.8); 7.1053 (0.7); 7.0958 (0.7); 7.0931 (0.7); 5.3002 (3.0); 4.9542 (7.5); 4.4219 (0.7); 4.4040 (0.7);
4.3948 (1.4); 4.3769 (1.4); 4.3509 (1.4); 4.3368 (1.5); 4.3238 (0.7); 4.3097 (0.7); 3.7262 (3.0); 3.6749 (16.0); 2.8479 (0.5); 2.8441
(0.7); 2.8300 (0.7); 2.8263 (0.5); 1.2053 (6.7); 1.1981 (1.6); 1.1874 (6.6); 1.1800 (1.5); −0.0002 (17.6); −0.0083 (0.6)
I-196: ¹H-NMR(400.6 MHz, CDCl3):
δ = 8.4836 (1.8); 8.4766 (1.8); 8.3793 (0.9); 8.3756 (1.7); 8.3720 (0.9); 8.0511 (0.7); 8.0490 (0.8); 8.0464 (0.9); 8.0443 (0.8);
8.0389 (0.8); 8.0368 (0.8); 8.0342 (0.8); 8.0321 (0.8); 7.7942 (0.6); 7.7895 (0.6); 7.7758 (0.8); 7.7735 (0.9); 7.7711 (0.7); 7.7688
(0.9); 7.7552 (1.0); 7.7504 (1.0); 7.6711 (1.0); 7.6688 (1.8); 7.6664 (1.0); 7.6505 (0.7); 7.6481 (1.2); 7.6457 (0.7); 7.5266 (0.8);
7.5223 (0.8); 7.5198 (0.8); 7.5154 (0.7); 7.5040 (0.8); 7.4997 (0.8); 7.4971 (0.8); 7.4928 (0.7); 7.2615 (13.9); 7.1164 (0.8); 7.1138
(0.8); 7.1043 (0.8); 7.1016 (0.9); 7.0981 (0.9); 7.0954 (0.8); 7.0859 (0.8); 7.0833 (0.8); 5.3002 (0.6); 5.2392 (1.7); 5.2218 (1.8);
4.4921 (1.0); 4.4864 (1.1); 4.4763 (2.2); 4.4709 (2.2); 4.4607 (1.1); 4.4552 (1.1); 3.6574 (16.0); 2.6909 (1.4); 2.6746 (2.2); 2.6592
(1.2); 1.7091 (6.2); 1.6916 (6.1); 1.5716 (1.3); 0.0079 (0.6); −0.0002 (20.0); −0.0085 (0.6)
I-198: ¹H-NMR(400.6 MHz, CDCl3):
δ = 8.4835 (3.4); 8.4766 (3.5); 8.3743 (1.7); 8.3704 (3.1); 8.3664 (1.7); 8.0535 (0.7); 8.0514 (0.8); 8.0488 (1.5); 8.0468 (1.5);
8.0442 (1.0); 8.0417 (1.3); 8.0393 (0.9); 8.0367 (1.5); 8.0346 (1.5); 8.0321 (0.9); 8.0300 (0.8); 7.7917 (0.6); 7.7869 (0.6); 7.7835
(0.6); 7.7787 (0.6); 7.7732 (0.7); 7.7710 (0.9); 7.7685 (0.7); 7.7659 (1.1); 7.7628 (1.0); 7.7604 (0.8); 7.7581 (0.9); 7.7526 (0.9);
7.7479 (0.9); 7.7445 (0.9); 7.7397 (0.8); 7.6641 (0.9); 7.6618 (1.7); 7.6592 (1.7); 7.6565 (1.7); 7.6542 (1.0); 7.6435 (0.7); 7.6411
(1.2); 7.6385 (1.3); 7.6359 (1.3); 7.6335 (0.7); 7.5256 (1.0); 7.5207 (1.1); 7.5145 (1.0); 7.5030 (1.0); 7.4985 (1.1); 7.4968 (1.0);
7.4919 (0.9); 7.2617 (21.9); 7.1137 (1.2); 7.1015 (1.2); 7.0956 (1.2); 7.0834 (1.1); 5.3002 (4.0); 5.2627 (1.6); 5.2545 (1.6); 5.2453
(1.6); 5.2370 (1.6); 4.4121 (0.9); 4.3941 (0.9); 4.3850 (1.3); 4.3671 (1.4); 4.3380 (2.2); 4.3347 (2.2); 4.3207 (2.7); 4.3197 (2.7);
4.2828 (1.4); 4.2687 (1.4); 4.2557 (0.9); 4.2417 (0.9); 3.8031 (0.9); 3.7261 (1.1); 3.6571 (15.9); 3.6502 (16.0); 2.8405 (0.5);
2.8382 (0.6); 2.8344 (0.8); 2.8231 (0.8); 2.8203 (0.9); 2.8165 (0.6); 2.8075 (0.8); 2.8024 (0.6); 1.7103 (6.7); 1.7081 (6.8); 1.6928
(6.6); 1.6907 (6.7); 1.5717 (1.8); 1.1980 (0.6); 1.1894 (6.6); 1.1797 (0.9); 1.1713 (12.4); 1.1531 (6.5); 0.0079 (0.9); −0.0002
(30.3); −0.0085 (0.9)
I-199: ¹H-NMR(400.6 MHz, CDCl3):
δ = 8.4978 (3.2); 8.4909 (3.3); 8.3822 (1.0); 8.3784 (1.9); 8.3752 (1.8); 8.3718 (1.8); 8.3681 (0.9); 8.0732 (1.3); 8.0704 (1.3);
8.0687 (1.4); 8.0610 (1.4); 8.0584 (1.2); 8.0565 (1.3); 7.8131 (0.6); 7.8086 (0.8); 7.8048 (0.6); 7.7947 (0.7); 7.7922 (1.1); 7.7887
(1.4); 7.7879 (1.4); 7.7841 (1.0); 7.7741 (1.0); 7.7703 (1.2); 7.7697 (1.3); 7.7659 (0.9); 7.7224 (1.1); 7.7198 (1.9); 7.7178 (1.8);
7.7158 (1.9); 7.7134 (1.1); 7.7017 (0.7); 7.6992 (1.1); 7.6972 (1.1); 7.6952 (1.2); 7.6927 (0.6); 7.5280 (0.8); 7.5235 (0.9); 7.5213
(1.1); 7.5169 (1.1); 7.5154 (0.9); 7.5110 (0.7); 7.5055 (0.8); 7.5010 (1.0); 7.4997 (1.1); 7.4944 (1.1); 7.4931 (0.9); 7.4885 (0.7);
7.2618 (26.7); 7.1494 (0.8); 7.1465 (1.0); 7.1455 (1.0); 7.1425 (0.9); 7.1372 (0.9); 7.1343 (1.2); 7.1332 (1.2); 7.1306 (1.5); 7.1272
(1.4); 7.1243 (1.2); 7.1189 (1.1); 7.1161 (1.2); 7.1151 (1.2); 7.1121 (1.0); 5.3526 (1.9); 5.3355 (3.0); 5.3184 (1.9); 5.3003 (4.6);
4.6601 (1.1); 4.6423 (1.6); 4.6247 (1.1); 3.7837 (16.0); 3.7236 (15.6); 1.7295 (7.1); 1.7273 (7.0); 1.7124 (7.1); 1.7103 (7.0);
1.5749 (4.4); 1.4953 (6.2); 1.4774 (6.1); 1.4460 (6.3); 1.4281 (6.2); 1.2635 (0.6); 0.8818 (1.1); 0.0080 (1.2); −0.0002
(37.2); −0.0085 (1.0)
I-200: ¹H-NMR(400.6 MHz, d₆-DMSO):
δ = 8.6539 (7.6); 8.6469 (7.8); 8.3755 (4.3); 8.3713 (7.8); 8.3675 (4.1); 8.1382 (3.3); 8.1356 (3.6); 8.1336 (3.3); 8.1279 (3.0);
8.1260 (3.6); 8.1234 (3.5); 8.1215 (3.3); 8.0430 (2.4); 8.0384 (2.2); 8.0243 (3.1); 8.0225 (3.4); 8.0198 (3.1); 8.0180 (3.2); 8.0039
(3.0); 7.9993 (2.8); 7.8648 (6.6); 7.8622 (8.0); 7.8488 (2.4); 7.8438 (11.0); 7.8408 (8.6); 7.8365 (3.6); 7.8321 (2.6); 7.8192 (2.8);
7.8149 (3.0); 7.8123 (2.7); 7.8080 (2.4); 7.7689 (6.0); 7.7484 (5.3); 7.6191 (1.9); 7.6005 (4.5); 7.5847 (1.8); 7.5818 (3.0); 7.4631
(6.4); 7.4590 (2.6); 7.4426 (8.8); 7.4283 (1.7); 7.4239 (4.5); 7.3733 (3.0); 7.3709 (3.1); 7.3612 (2.8); 7.3588 (3.0); 7.3548 (3.1);
7.3524 (2.9); 7.3426 (2.9); 7.3403 (2.8); 5.7558 (3.0); 4.0427 (16.0); 3.6150 (0.6); 3.3497 (78.5); 3.1844 (2.1); 3.1567 (0.8);
3.1461 (0.8); 3.1381 (0.7); 3.1280 (0.7); 2.6754 (1.1); 2.6708 (1.4); 2.6663 (1.1); 2.5413 (2.9); 2.5343 (0.8); 2.5246 (3.9); 2.5199
(5.7); 2.5111 (82.3); 2.5065 (176.2); 2.5020 (243.6); 2.4974 (170.7); 2.4929 (77.4); 2.3337 (1.0); 2.3290 (1.5); 2.3245 (1.0);
1.4871 (0.5); 1.2716 (3.6); 1.2578 (5.2); 1.2418 (6.1); 1.2234 (1.1); 0.9599 (0.7); 0.9419 (1.2); 0.9288 (0.9); 0.0080 (3.8); −0.0002
(125.7); −0.0085 (4.0); −0.1496 (0.5)
I-201: ¹H-NMR(400.6 MHz, CDCl3):
δ = 8.5097 (3.7); 8.5028 (3.7); 8.3428 (2.2); 8.3391 (3.8); 8.3354 (2.2); 8.1471 (1.5); 8.1452 (1.7); 8.1426 (1.8); 8.1405 (1.7);
8.1351 (1.6); 8.1330 (1.8); 8.1304 (1.8); 8.1284 (1.7); 7.8619 (1.3); 7.8572 (1.2); 7.8433 (1.6); 7.8414 (2.0); 7.8387 (1.6); 7.8368
(1.9); 7.8229 (1.9); 7.8182 (1.8); 7.7289 (2.1); 7.7266 (3.7); 7.7244 (2.3); 7.7083 (1.6); 7.7061 (2.6); 7.7039 (1.6); 7.5045 (1.4);
7.5001 (1.6); 7.4977 (1.6); 7.4933 (1.5); 7.4823 (1.5); 7.4779 (1.6); 7.4754 (1.6); 7.4710 (1.5); 7.2635 (13.4); 7.2295 (1.6); 7.2270
(1.7); 7.2174 (1.6); 7.2148 (1.7); 7.2110 (1.7); 7.2084 (1.6); 7.1989 (1.6); 7.1963 (1.6); 7.1359 (0.9); 3.7824 (16.0); 2.7607 (0.6);
2.7521 (1.1); 2.7429 (1.6); 2.7342 (1.6); 2.7250 (1.2); 2.7164 (0.7); 0.7836 (1.0); 0.7698 (2.7); 0.7663 (4.0); 0.7637 (1.9); 0.7524
(3.6); 0.7485 (3.0); 0.7458 (1.6); 0.7351 (1.2); 0.4874 (1.2); 0.4774 (2.5); 0.4744 (2.9); 0.4708 (2.8); 0.4685 (2.6); 0.4648 (3.0);
0.4612 (2.8); 0.4476 (1.0); 0.0080 (0.5); −0.0002 (20.1); −0.0085 (0.6)
I-202: ¹H-NMR(400.6 MHz, CDCl3):
δ = 8.4689 (1.3); 8.4652 (1.3); 8.4569 (1.3); 8.4533 (1.3); 8.4276 (3.2); 8.4207 (3.2); 8.2834 (1.8); 8.2797 (3.1); 8.2760 (1.7);
8.1605 (1.4); 8.1565 (1.4); 8.1407 (1.5); 8.1364 (1.4); 7.5302 (1.3); 7.5258 (1.3); 7.5233 (1.4); 7.5189 (1.3); 7.5080 (1.3); 7.5036
(1.4); 7.5011 (1.3); 7.4967 (1.2); 7.4564 (1.0); 7.4547 (1.1); 7.4444 (1.0); 7.4427 (1.1); 7.4367 (1.0); 7.4350 (1.0); 7.4247 (0.9);
7.4230 (1.0); 7.2616 (16.3); 4.8798 (14.9); 4.2809 (1.9); 4.2630 (5.9); 4.2452 (6.0); 4.2274 (1.9); 1.5640 (8.0); 1.4638 (1.0);
1.4400 (1.0); 1.2998 (7.8); 1.2819 (16.0); 1.2756 (0.7); 1.2641 (8.5); 0.8986 (0.6); 0.8818 (2.3); 0.8641 (0.9); 0.0079 (0.7); −0.0002
(23.6); −0.0085 (0.6)
I-203: ¹H-NMR(400.6 MHz, CDCl3):
δ = 8.4739 (3.0); 8.4670 (3.1); 8.2882 (1.6); 8.2844 (3.0); 8.2807 (1.7); 7.5855 (1.0); 7.5705 (1.1); 7.5640 (1.5); 7.5495 (1.4);
7.5432 (1.1); 7.5282 (1.1); 7.4116 (1.3); 7.4072 (1.3); 7.4047 (1.3); 7.4003 (1.2); 7.3896 (1.3); 7.3852 (1.4); 7.3826 (1.3); 7.3783
(1.2); 7.2615 (20.6); 6.8660 (1.1); 6.8599 (1.2); 6.8566 (1.2); 6.8505 (1.2); 6.8442 (1.0); 6.8382 (1.1); 6.8349 (1.1); 6.8287 (1.0);
6.3692 (1.8); 6.3404 (2.2); 6.3251 (2.8); 6.2962 (2.6); 6.0335 (2.7); 6.0289 (2.8); 5.9893 (2.1); 5.9848 (2.0); 5.3362 (2.6); 5.3317
(2.3); 5.3074 (2.3); 5.3028 (2.7); 5.2840 (3.2); 5.2666 (3.2); 5.2493 (0.8); 4.2719 (1.9); 4.2541 (6.2); 4.2363 (6.4); 4.2185 (2.1);
1.6996 (12.0); 1.6823 (11.8); 1.5670 (4.2); 1.2927 (7.6); 1.2749 (16.0); 1.2571 (7.5); 0.0079 (0.8); −0.0002 (30.0); −0.0085 (0.8)
I-204: ¹H-NMR(400.6 MHz, d₆-DMSO):
δ = 13.0682 (0.9); 8.5970 (4.6); 8.5900 (4.6); 8.3152 (2.4); 8.3111 (4.5); 8.3074 (2.2); 8.1670 (1.6); 8.1656 (1.7); 8.1626 (1.9);
8.1611 (1.7); 8.1552 (1.7); 8.1538 (1.8); 8.1508 (1.8); 8.1493 (1.7); 7.8816 (1.5); 7.8797 (1.6); 7.8771 (1.7); 7.8753 (1.5); 7.8625
(1.7); 7.8607 (1.9); 7.8581 (1.7); 7.8563 (1.6); 7.7414 (1.6); 7.7371 (1.7); 7.7345 (1.7); 7.7301 (1.6); 7.7176 (1.7); 7.7133 (1.9);
7.7107 (1.6); 7.7063 (1.6); 7.3928 (2.3); 7.3810 (2.2); 7.3740 (2.2); 7.3621 (2.1); 5.7571 (3.0); 4.8447 (12.6); 3.3243 (2.9); 2.5247
(0.8); 2.5201 (1.0); 2.5113 (14.4); 2.5067 (31.7); 2.5022 (44.5); 2.4976 (30.6); 2.4930 (13.3); 2.2468 (16.0); 1.9889 (0.6); 0.0079
(1.4); −0.0002 (47.8); −0.0085 (1.2)
I-205: ¹H-NMR(400.6 MHz, CDCl3):
δ = 8.5204 (0.7); 8.5168 (0.8); 8.5085 (0.8); 8.5048 (0.8); 8.4435 (1.4); 8.4367 (1.4); 8.3042 (0.9); 8.3006 (1.5); 8.2971 (0.9);
8.1778 (0.8); 8.1735 (0.8); 8.1577 (0.9); 8.1536 (0.8); 7.5267 (0.7); 7.5223 (0.8); 7.5198 (0.8); 7.5154 (0.7); 7.5045 (0.9); 7.5002
(1.4); 7.4978 (1.0); 7.4933 (0.8); 7.4901 (0.6); 7.4882 (0.6); 7.4823 (0.6); 7.4805 (0.6); 7.4702 (0.6); 7.4685 (0.6); 7.2623 (11.6);
4.8669 (7.0); 4.1743 (3.2); 4.1611 (3.2); 3.7991 (16.0); −0.0002 (16.8)
I-206: ¹H-NMR(400.6 MHz, CDCl3):
δ = 8.4722 (0.8); 8.4686 (0.8); 8.4602 (0.8); 8.4565 (0.8); 8.4302 (1.2); 8.4235 (1.2); 8.2825 (1.3); 8.1623 (0.8); 8.1584 (0.9);
8.1424 (0.9); 8.1385 (0.9); 7.5305 (0.7); 7.5261 (0.8); 7.5236 (0.8); 7.5192 (0.8); 7.5083 (0.7); 7.5039 (0.8); 7.5014 (0.8); 7.4970
(0.7); 7.4605 (0.6); 7.4587 (0.7); 7.4485 (0.6); 7.4467 (0.6); 7.4407 (0.6); 7.4389 (0.6); 7.4287 (0.6); 7.4269 (0.6); 7.2608 (22.8);
7.2559 (0.5); 4.8958 (8.6); 3.7931 (16.0); 1.5626 (3.1); 0.0080 (0.8); −0.0002 (32.8); −0.0085 (0.9)
I-207: ¹H-NMR(400.6 MHz, d₆-DMSO):
δ = 13.1181 (1.3); 8.6952 (5.9); 8.6883 (5.8); 8.4334 (3.0); 8.4293 (5.5); 8.4257 (2.8); 8.1989 (1.2); 8.1835 (1.3); 8.1768 (2.5);
8.1614 (2.5); 8.1547 (1.4); 8.1393 (1.3); 7.9290 (2.2); 7.9246 (2.2); 7.9220 (2.2); 7.9177 (2.0); 7.9053 (2.3); 7.9009 (2.5); 7.8984
(2.1); 7.8940 (2.0); 7.4045 (1.4); 7.3981 (1.9); 7.3964 (1.8); 7.3899 (1.5); 7.3824 (1.3); 7.3759 (1.6); 7.3743 (1.6); 7.3678 (1.2);
5.7569 (3.5); 4.8758 (16.0); 3.3209 (18.5); 2.6750 (0.6); 2.6704 (0.8); 2.6658 (0.6); 2.5409 (0.8); 2.5242 (2.3); 2.5195 (3.0);
2.5108 (43.7); 2.5062 (97.7); 2.5016 (139.0); 2.4970 (95.6); 2.4924 (41.8); 2.3333 (0.6); 2.3287 (0.8); 2.3240 (0.6); 0.0080 (3.6);
0.0064 (0.7); 0.0055 (0.8); 0.0047 (1.0); 0.0039 (1.3); 0.0031 (2.4); 0.0023 (4.3); 0.0015 (6.8); −0.0002 (133.1); −0.0018
(8.6); −0.0027 (5.6); −0.0035 (3.8); −0.0043 (2.3); −0.0051 (1.8); −0.0060 (1.5); −0.0067 (1.3); −0.0076 (1.4); −0.0085 (3.9); −0.0098
(0.9); −0.0107 (0.6); −0.0115 (0.6); −0.0123 (0.5)
I-208: ¹H-NMR(400.6 MHz, CDCl3):
δ = 8.4840 (1.6); 8.4776 (1.7); 8.2957 (2.0); 7.6297 (1.0); 7.6149 (1.1); 7.6094 (1.4); 7.6081 (1.4); 7.5945 (1.4); 7.5932 (1.3);
7.5877 (1.2); 7.5729 (1.1); 7.5400 (1.2); 7.5356 (1.3); 7.5331 (1.3); 7.5288 (1.2); 7.5179 (1.2); 7.5135 (1.3); 7.5111 (1.3); 7.5067
(1.2); 7.2617 (19.6); 6.9393 (1.1); 6.9331 (1.3); 6.9300 (1.2); 6.9238 (1.2); 6.9174 (1.1); 6.9112 (1.1); 6.9082 (1.1); 6.9020 (1.0);
5.2509 (0.8); 5.2335 (3.2); 5.2161 (3.2); 5.1987 (0.9); 4.2674 (2.2); 4.2496 (7.0); 4.2318 (7.0); 4.2140 (2.2); 1.6990 (12.2); 1.6816
(12.1); 1.5664 (6.3); 1.2881 (7.7); 1.2703 (16.0); 1.2525 (7.4); 0.0080 (0.8); −0.0002 (28.8); −0.0085 (0.9)
I-209: ¹H-NMR(400.6 MHz, CDCl3):
δ = 8.4318 (2.7); 8.4249 (2.8); 8.2763 (1.6); 8.2725 (2.8); 8.2689 (1.6); 7.5787 (1.0); 7.5637 (1.1); 7.5581 (1.3); 7.5570 (1.4);
7.5431 (1.3); 7.5420 (1.4); 7.5366 (1.2); 7.5216 (1.1); 7.4204 (1.2); 7.4161 (1.2); 7.4134 (1.3); 7.4092 (1.2); 7.3982 (1.2); 7.3939
(1.3); 7.3913 (1.3); 7.3870 (1.2); 7.2610 (34.4); 6.8643 (1.1); 6.8582 (1.2); 6.8549 (1.2); 6.8488 (1.2); 6.8425 (1.0); 6.8364 (1.1);
6.8332 (1.1); 6.8270 (1.1); 5.2676 (0.8); 5.2503 (3.1); 5.2329 (3.2); 5.2156 (0.9); 5.1283 (0.6); 5.1244 (2.1); 5.1202 (3.0); 5.1161
(2.3); 5.1122 (0.7); 5.0317 (2.2); 5.0294 (2.8); 5.0271 (2.7); 5.0248 (2.1); 4.2551 (2.0); 4.2373 (6.6); 4.2196 (6.7); 4.2018 (2.2);
1.9331 (7.4); 1.9308 (9.8); 1.9295 (9.9); 1.9272 (8.0); 1.6594 (11.6); 1.6526 (1.0); 1.6420 (11.6); 1.6354 (0.9); 1.6278 (1.0);
1.5632 (2.7); 1.2825 (7.7); 1.2738 (0.7); 1.2648 (16.0); 1.2570 (1.1); 1.2470 (7.4); 0.0080 (1.4); −0.0002 (48.6); −0.0085 (1.4)
I-210: ¹H-NMR(400.6 MHz, CDCl3):
δ = 8.4940 (0.8); 8.4878 (0.8); 8.3097 (1.0); 7.6292 (0.6); 7.6144 (0.6); 7.6091 (0.7); 7.6074 (0.8); 7.5942 (0.8); 7.5926 (0.8);
7.5874 (0.7); 7.5725 (0.6); 7.5536 (0.6); 7.5492 (0.7); 7.5467 (0.7); 7.5423 (0.6); 7.5316 (0.7); 7.5272 (0.7); 7.5248 (0.7); 7.5203
(0.7); 7.2610 (22.1); 6.9597 (0.6); 6.9534 (0.7); 6.9504 (0.7); 6.9441 (0.7); 6.9378 (0.6); 6.9315 (0.7); 6.9285 (0.7); 6.9223 (0.6);
4.9456 (8.6); 3.8096 (16.0); 1.5521 (9.2); 0.0080 (0.8); −0.0002 (30.3); −0.0085 (0.8)
I-211: ¹H-NMR(400.6 MHz, CDCl3):
δ = 8.3992 (2.4); 8.3924 (2.4); 8.2673 (1.4); 8.2636 (2.5); 8.2600 (1.5); 8.2060 (1.1); 8.2047 (1.2); 8.2016 (1.3); 8.2003 (1.2);
8.1942 (1.2); 8.1928 (1.2); 8.1898 (1.2); 8.1884 (1.1); 7.6467 (1.1); 7.6448 (1.2); 7.6422 (1.2); 7.6405 (1.1); 7.6276 (1.2); 7.6258
(1.4); 7.6232 (1.3); 7.6214 (1.2); 7.5157 (1.2); 7.5113 (1.2); 7.5088 (1.3); 7.5044 (1.2); 7.4931 (1.2); 7.4887 (1.3); 7.4862 (1.2);
7.4818 (1.2); 7.2624 (12.2); 7.2392 (1.6); 7.2273 (1.6); 7.2202 (1.5); 7.2084 (1.4); 4.8984 (14.6); 4.2787 (1.8); 4.2608 (5.9);
4.2430 (5.9); 4.2252 (1.9); 2.2154 (13.5); 1.5807 (7.7); 1.4372 (1.0); 1.3029 (7.8); 1.2851 (16.0); 1.2672 (8.3); 0.8987 (0.6);
0.8818 (2.2); 0.8641 (0.8); −0.0002 (17.1)
I-212: ¹H-NMR(400.6 MHz, CDCl3):
δ = 8.3996 (1.3); 8.3927 (1.3); 8.2641 (0.7); 8.2603 (1.3); 8.2566 (0.7); 8.2011 (0.5); 8.1997 (0.5); 8.1967 (0.6); 8.1953 (0.5);
8.1892 (0.5); 8.1878 (0.6); 8.1848 (0.6); 8.1834 (0.5); 7.6444 (0.5); 7.6418 (0.6); 7.6272 (0.5); 7.6254 (0.6); 7.6228 (0.6); 7.6210
(0.5); 7.5150 (0.6); 7.5106 (0.6); 7.5081 (0.6); 7.5037 (0.6); 7.4924 (0.6); 7.4880 (0.6); 7.4855 (0.6); 7.4811 (0.5); 7.2627 (5.4);
7.2376 (0.7); 7.2257 (0.7); 7.2186 (0.7); 7.2067 (0.7); 4.9504 (6.4); 4.3496 (1.6); 4.3412 (0.8); 4.3379 (1.6); 4.3342 (0.8); 4.3260
(1.7); 3.6202 (1.9); 3.6134 (0.7); 3.6121 (0.9); 3.6084 (1.7); 3.6051 (0.9); 3.6037 (0.6); 3.5967 (1.8); 3.4875 (1.0); 3.3574 (16.0);
2.2174 (5.9); −0.0002 (8.0)
I-213: ¹H-NMR(400.6 MHz, CDCl3):
δ = 8.4834 (2.7); 8.4768 (2.9); 8.2941 (3.2); 7.6030 (0.7); 7.5814 (1.4); 7.5664 (1.4); 7.5458 (0.8); 7.4363 (1.2); 7.4312 (1.5);
7.4253 (1.3); 7.4145 (1.3); 7.4096 (1.6); 7.4032 (1.2); 7.2608 (29.5); 6.9037 (0.6); 6.8942 (1.2); 6.8881 (1.3); 6.8786 (0.9); 6.8724
(1.2); 6.8663 (1.2); 6.8568 (0.6); 5.2956 (1.3); 5.2782 (1.3); 5.2439 (0.5); 5.2264 (1.6); 5.2090 (1.7); 5.1917 (0.5); 3.7802 (16.0);
3.7559 (12.5); 2.4370 (0.9); 2.4298 (0.7); 2.4170 (0.6); 2.4081 (1.4); 2.4006 (0.5); 2.3878 (1.2); 2.3787 (0.8); 2.3586 (0.6); 1.9197
(0.8); 1.8997 (0.8); 1.8888 (0.6); 1.7903 (0.6); 1.7782 (0.9); 1.7623 (1.5); 1.7490 (1.4); 1.7424 (0.9); 1.7341 (1.1); 1.7196 (0.8);
1.7144 (0.6); 1.6999 (0.6); 1.6861 (0.5); 1.6638 (5.5); 1.6468 (11.7); 1.6297 (7.0); 1.5505 (10.2); 0.0079 (1.7); −0.0002
(42.3); −0.0085 (2.0)
I-214: ¹H-NMR(400.6 MHz, d₆-DMSO):
δ = 13.0004 (0.6); 8.6175 (5.0); 8.6105 (5.2); 8.3408 (2.6); 8.3366 (5.0); 8.3328 (2.6); 8.1530 (1.1); 8.1375 (1.2); 8.1310 (2.2);
8.1155 (2.2); 8.1090 (1.2); 8.0935 (1.1); 7.8213 (1.6); 7.8170 (1.7); 7.8144 (1.8); 7.8101 (1.6); 7.7976 (1.7); 7.7933 (1.9); 7.7907
(1.7); 7.7864 (1.6); 7.3408 (1.2); 7.3343 (1.7); 7.3326 (1.7); 7.3260 (1.4); 7.3186 (1.2); 7.3122 (1.5); 7.3104 (1.5); 7.3040 (1.1);
5.7568 (2.5); 5.1135 (2.4); 5.1087 (3.5); 5.1047 (2.7); 5.0002 (3.0); 4.9979 (3.6); 4.9952 (3.5); 4.9929 (2.9); 4.8290 (14.6); 4.8149
(0.9); 3.3234 (16.0); 2.6708 (0.5); 2.5244 (1.5); 2.5198 (2.0); 2.5110 (29.4); 2.5065 (65.3); 2.5019 (92.4); 2.4973 (64.4); 2.4928
(28.9); 2.3290 (0.5); 1.8903 (9.6); 1.8881 (14.2); 1.8870 (14.5); 1.8848 (10.3); 0.0080 (1.8); −0.0002 (70.5); −0.0085 (2.0)
I-215: ¹H-NMR(400.0 MHz, CDCl3):
δ = 8.4404 (1.9); 8.4335 (1.8); 8.2790 (2.0); 7.5887 (0.6); 7.5739 (0.7); 7.5675 (1.1); 7.5527 (1.0); 7.5469 (0.8); 7.5320 (0.6);
7.4226 (0.7); 7.4159 (0.8); 7.4113 (0.7); 7.4002 (0.7); 7.3961 (0.8); 7.3892 (0.7); 7.2606 (32.7); 6.8950 (0.8); 6.8859 (0.8); 6.8794
(0.8); 6.8731 (0.8); 6.8641 (0.8); 6.8575 (0.6); 5.1515 (1.4); 5.1476 (2.1); 5.1436 (1.4); 5.0531 (2.0); 4.9599 (0.6); 4.9369 (1.0);
4.9285 (9.5); 3.8146 (1.0); 3.7995 (16.0); 3.7943 (3.5); 1.9220 (7.3); 1.5486 (14.2); 0.0079 (1.8); −0.0002 (42.6); −0.0084 (1.3)
I-216: ¹H-NMR(400.6 MHz, CDCl3):
δ = 8.4927 (1.7); 8.4858 (1.8); 8.3056 (1.0); 8.3019 (1.8); 8.2982 (1.0); 7.6374 (0.6); 7.6226 (0.6); 7.6173 (0.8); 7.6157 (0.8);
7.6025 (0.8); 7.6008 (0.8); 7.5955 (0.7); 7.5807 (0.6); 7.5453 (0.7); 7.5409 (0.8); 7.5384 (0.8); 7.5340 (0.8); 7.5233 (0.8); 7.5189
(0.8); 7.5165 (0.8); 7.5120 (0.7); 7.2618 (8.6); 6.9661 (0.7); 6.9598 (0.7); 6.9569 (0.7); 6.9506 (0.7); 6.9442 (0.6); 6.9379 (0.7);
6.9350 (0.7); 6.9287 (0.6); 4.9480 (8.8); 3.8118 (16.0); 1.5750 (0.7); −0.0002 (13.3)
I-217: ¹H-NMR(400.6 MHz, d₆-DMSO):
δ = 12.8988 (0.8); 8.6666 (4.7); 8.6597 (4.7); 8.4273 (2.4); 8.4230 (4.6); 8.4195 (2.2); 8.1574 (1.8); 8.1554 (2.0); 8.1528 (2.2);
8.1508 (2.0); 8.1454 (1.9); 8.1433 (2.1); 8.1408 (2.2); 8.1387 (1.9); 8.0570 (1.7); 8.0523 (1.6); 8.0384 (2.1); 8.0365 (2.2); 8.0337
(1.9); 8.0318 (2.0); 8.0179 (2.2); 8.0132 (2.0); 7.8920 (1.8); 7.8877 (1.8); 7.8850 (1.8); 7.8808 (1.6); 7.8680 (1.8); 7.8637 (1.9);
7.8610 (1.7); 7.8567 (1.6); 7.8204 (2.3); 7.8181 (4.1); 7.8159 (2.3); 7.7999 (2.0); 7.7976 (3.5); 7.7953 (1.8); 7.3709 (2.1); 7.3684
(2.1); 7.3588 (1.9); 7.3563 (2.0); 7.3524 (2.0); 7.3499 (1.8); 7.3402 (2.0); 7.3377 (1.9); 5.7571 (4.3); 4.0346 (16.0); 3.3284 (8.6);
2.5251 (1.0); 2.5205 (1.2); 2.5118 (13.4); 2.5072 (28.7); 2.5026 (39.7); 2.4980 (27.0); 2.4935 (11.5); 1.6731 (1.3); 0.0079
(1.0); −0.0002 (33.8); −0.0085 (0.9)
I-218: ¹H-NMR(400.6 MHz, d₆-DMSO):
δ = 13.0976 (1.1); 8.6633 (6.8); 8.6563 (7.0); 8.4314 (3.7); 8.4272 (7.0); 8.4232 (3.3); 8.1024 (2.6); 8.1004 (2.9); 8.0978 (3.3);
8.0957 (3.0); 8.0903 (2.7); 8.0883 (3.2); 8.0857 (3.2); 8.0836 (3.0); 8.0095 (2.5); 8.0048 (2.3); 7.9909 (3.0); 7.9889 (3.1); 7.9862
(2.8); 7.9842 (3.0); 7.9703 (3.1); 7.9656 (2.9); 7.8933 (2.6); 7.8891 (2.6); 7.8864 (2.6); 7.8821 (2.4); 7.8693 (2.7); 7.8650 (2.9);
7.8623 (2.5); 7.8581 (2.4); 7.6770 (3.1); 7.6748 (5.8); 7.6726 (3.3); 7.6564 (2.9); 7.6542 (5.3); 7.6519 (2.9); 7.3017 (3.0); 7.2992
(3.0); 7.2895 (2.7); 7.2871 (3.0); 7.2832 (3.0); 7.2807 (2.7); 7.2710 (2.8); 7.2685 (2.8); 5.1891 (1.2); 5.1718 (6.0); 5.1544 (6.2);
5.1370 (1.2); 4.0382 (0.6); 4.0205 (0.6); 3.3263 (6.8); 2.5250 (1.0); 2.5203 (1.3); 2.5115 (18.4); 2.5070 (39.9); 2.5024 (55.8);
2.4978 (38.0); 2.4932 (16.5); 1.9890 (2.6); 1.9097 (0.8); 1.6088 (16.0); 1.5914 (15.9); 1.3561 (0.6); 1.1924 (0.8); 1.1746 (1.6);
1.1569 (0.8); 0.0080 (1.7); 0.0048 (0.6); 0.0040 (0.8); 0.0023 (2.3); −0.0002 (56.8); −0.0041 (1.0); −0.0050 (0.7); −0.0059
(0.5); −0.0085 (1.5)
I-219: ¹H-NMR(400.6 MHz, CDCl3):
δ = 8.4930 (4.5); 8.4861 (4.6); 8.3014 (2.8); 8.2978 (4.7); 8.2943 (3.0); 7.5817 (1.6); 7.5667 (1.6); 7.5602 (2.3); 7.5454 (2.3);
7.5394 (1.7); 7.5244 (1.7); 7.5193 (0.5); 7.4447 (1.9); 7.4404 (2.1); 7.4379 (2.1); 7.4335 (1.9); 7.4229 (2.0); 7.4186 (2.2); 7.4160
(2.0); 7.4117 (1.8); 7.2682 (0.5); 7.2608 (52.1); 6.8774 (1.7); 6.8713 (1.9); 6.8682 (1.9); 6.8620 (1.8); 6.8556 (1.6); 6.8495 (1.8);
6.8463 (1.8); 6.8402 (1.5); 6.3625 (2.7); 6.3336 (3.3); 6.3184 (4.0); 6.2895 (3.7); 6.0014 (3.9); 5.9971 (4.1); 5.9573 (3.1); 5.9529
(3.1); 5.3801 (1.3); 5.3628 (5.1); 5.3453 (5.8); 5.3437 (5.2); 5.3393 (3.8); 5.3280 (1.4); 5.3147 (3.4); 5.3103 (3.7); 5.3002 (0.6);
1.7525 (16.0); 1.7351 (15.8); 1.7109 (0.9); 1.4322 (1.5); 1.2641 (2.1); 0.8989 (1.1); 0.8820 (3.9); 0.8643 (1.5); 0.0080
(2.2); −0.0002 (75.6); −0.0085 (2.7)
I-220: ¹H-NMR(400.6 MHz, CDCl3):
δ = 8.4601 (3.2); 8.4532 (3.3); 8.2822 (1.8); 8.2783 (3.3); 8.2744 (1.8); 7.9906 (1.2); 7.9885 (1.4); 7.9859 (1.5); 7.9838 (1.4);
7.9785 (1.3); 7.9764 (1.4); 7.9738 (1.5); 7.9717 (1.4); 7.7065 (1.2); 7.7017 (1.2); 7.6883 (1.2); 7.6858 (1.7); 7.6836 (1.4); 7.6810
(1.7); 7.6676 (1.7); 7.6629 (1.7); 7.6136 (1.7); 7.6111 (3.1); 7.6087 (2.0); 7.5929 (1.2); 7.5905 (1.9); 7.5880 (1.1); 7.3466 (1.2);
7.3424 (1.3); 7.3397 (1.3); 7.3355 (1.2); 7.3240 (1.3); 7.3197 (1.3); 7.3171 (1.3); 7.3128 (1.2); 7.2613 (19.6); 7.0165 (1.5); 7.0138
(1.4); 7.0044 (1.4); 7.0016 (1.4); 6.9983 (1.5); 6.9956 (1.3); 6.9862 (1.4); 6.9834 (1.3); 4.9659 (15.0); 4.9229 (0.6); 4.3199 (1.9);
4.3020 (6.1); 4.2842 (6.2); 4.2664 (2.0); 4.1713 (0.8); 4.1685 (1.3); 3.2503 (0.6); 3.2296 (1.1); 3.2279 (1.0); 3.2051 (0.7); 2.5058
(1.2); 2.5002 (0.9); 2.4815 (1.6); 2.4754 (1.6); 2.4574 (0.9); 2.4530 (1.0); 2.4508 (1.0); 2.4283 (0.5); 2.1251 (0.6); 2.1182 (0.7);
2.1101 (0.8); 2.1032 (1.0); 2.0976 (1.4); 2.0921 (0.8); 2.0893 (1.0); 2.0831 (1.0); 2.0759 (1.4); 2.0698 (0.9); 2.0630 (0.5); 2.0557
(0.6); 1.9021 (0.6); 1.8832 (0.9); 1.8771 (0.9); 1.8729 (0.5); 1.8637 (1.1); 1.8573 (1.7); 1.8511 (0.9); 1.8430 (1.2); 1.8407 (1.3);
1.8328 (1.2); 1.8204 (0.8); 1.8181 (0.7); 1.8146 (0.7); 1.3371 (7.8); 1.3193 (16.0); 1.3015 (7.5); 1.2542 (0.9); 0.0079 (0.7); −0.0002
(30.3); −0.0085 (0.9)
I-221: ¹H-NMR(400.6 MHz, CDCl3):
δ = 8.4890 (1.2); 8.4821 (1.2); 8.3740 (0.8); 8.3704 (1.3); 8.3669 (0.8); 8.0752 (0.7); 8.0731 (0.7); 8.0705 (0.8); 8.0684 (0.7);
8.0630 (0.7); 8.0609 (0.8); 8.0583 (0.8); 8.0562 (0.7); 7.7957 (0.6); 7.7910 (0.5); 7.7773 (0.7); 7.7750 (0.8); 7.7726 (0.7); 7.7703
(0.8); 7.7567 (0.8); 7.7519 (0.8); 7.6710 (0.9); 7.6687 (1.5); 7.6664 (0.9); 7.6504 (0.7); 7.6481 (1.1); 7.6457 (0.6); 7.5287 (0.6);
7.5244 (0.7); 7.5218 (0.7); 7.5175 (0.6); 7.5062 (0.6); 7.5019 (0.7); 7.4993 (0.6); 7.4950 (0.6); 7.2612 (12.9); 7.1330 (0.8); 7.1304
(0.7); 7.1208 (0.7); 7.1182 (0.8); 7.1146 (0.8); 7.1120 (0.7); 7.1024 (0.7); 7.0998 (0.7); 5.3002 (1.7); 4.9553 (7.0); 4.4231 (0.6);
4.4052 (0.6); 4.3960 (1.3); 4.3781 (1.4); 4.3526 (1.3); 4.3386 (1.4); 4.3255 (0.6); 4.3115 (0.6); 3.7262 (0.7); 3.6748 (16.0); 2.8459
(0.7); 2.8318 (0.7); 1.5629 (0.8); 1.2066 (6.4); 1.1981 (0.5); 1.1887 (6.4); 0.0079 (0.6); −0.0002 (19.5); −0.0028 (0.9); −0.0085 (0.6)
I-222: ¹H-NMR(400.6 MHz, CDCl3):
δ = 8.4342 (1.9); 8.4273 (1.9); 8.2797 (1.0); 8.2758 (1.8); 8.2721 (1.0); 7.5853 (0.6); 7.5703 (0.6); 7.5647 (0.7); 7.5636 (0.8);
7.5497 (0.8); 7.5486 (0.7); 7.5432 (0.6); 7.5281 (0.6); 7.4246 (0.7); 7.4203 (0.7); 7.4176 (0.7); 7.4133 (0.6); 7.4024 (0.7); 7.3981
(0.7); 7.3955 (0.7); 7.3912 (0.6); 7.2620 (9.0); 6.8664 (0.6); 6.8603 (0.7); 6.8570 (0.6); 6.8509 (0.6); 6.8446 (0.6); 6.8385 (0.6);
6.8352 (0.6); 6.8291 (0.6); 5.2656 (1.7); 5.2483 (1.7); 5.1256 (1.2); 5.1215 (1.6); 5.1174 (1.2); 5.0324 (1.2); 5.0301 (1.6); 5.0278
(1.5); 5.0255 (1.2); 3.7719 (16.0); 3.7663 (1.1); 1.9278 (4.1); 1.9255 (5.4); 1.9241 (5.3); 1.9218 (4.3); 1.6633 (6.4); 1.6576 (0.6);
1.6460 (6.4); 1.6402 (0.6); 1.6315 (0.5); −0.0002 (12.6); −0.0027 (0.7)
I-223: ¹H-NMR(400.6 MHz, CDCl3):
δ = 8.4619 (0.8); 8.4582 (0.9); 8.4500 (0.8); 8.4462 (0.9); 8.4295 (0.8); 8.4232 (0.9); 8.2936 (1.0); 8.1479 (0.9); 8.1437 (0.9);
8.1281 (0.9); 8.1239 (0.9); 7.5184 (0.7); 7.5140 (0.7); 7.5115 (0.7); 7.5072 (0.7); 7.4962 (0.7); 7.4919 (0.8); 7.4894 (0.7); 7.4850
(0.7); 7.4448 (0.6); 7.4430 (0.7); 7.4328 (0.6); 7.4310 (0.7); 7.4250 (0.6); 7.4233 (0.6); 7.4130 (0.6); 7.4113 (0.6); 7.2618 (8.8);
5.3003 (1.7); 4.8886 (8.7); 3.7888 (16.0); 1.5754 (0.5); 0.0698 (0.8); −0.0002 (13.2)
I-224: ¹H-NMR(400.6 MHz, CDCl3):
δ = 8.4802 (2.9); 8.4733 (3.0); 8.2923 (1.7); 8.2885 (3.0); 8.2848 (1.9); 7.5909 (1.0); 7.5759 (1.1); 7.5692 (1.5); 7.5543 (1.4);
7.5488 (1.2); 7.5338 (1.1); 7.4140 (1.2); 7.4097 (1.3); 7.4072 (1.4); 7.4028 (1.3); 7.3922 (1.3); 7.3878 (1.4); 7.3853 (1.4); 7.3809
(1.3); 7.2609 (33.3); 6.8954 (1.1); 6.8893 (1.2); 6.8861 (1.3); 6.8799 (1.2); 6.8736 (1.1); 6.8675 (1.1); 6.8643 (1.2); 6.8581 (1.1);
6.3772 (1.8); 6.3484 (2.1); 6.3331 (2.7); 6.3042 (2.5); 6.0494 (2.7); 6.0451 (2.9); 6.0052 (2.1); 6.0009 (2.1); 5.3567 (2.6); 5.3524
(2.4); 5.3278 (2.2); 5.3235 (2.5); 4.9551 (15.6); 4.3086 (2.0); 4.2908 (6.0); 4.2729 (6.1); 4.2551 (2.0); 1.5545 (8.8); 1.3237 (7.7);
1.3059 (16.0); 1.2954 (0.6); 1.2881 (7.6); 0.0079 (1.5); −0.0002 (47.9); −0.0085 (1.7)
I-225: ¹H-NMR(400.6 MHz, CDCl3):
δ = 8.4587 (1.3); 8.4551 (1.4); 8.4467 (1.4); 8.4429 (1.4); 8.4272 (2.7); 8.4203 (2.8); 8.2941 (1.6); 8.2904 (2.8); 8.2868 (1.6);
8.1457 (1.4); 8.1417 (1.5); 8.1259 (1.6); 8.1218 (1.5); 7.5186 (1.2); 7.5142 (1.3); 7.5117 (1.3); 7.5073 (1.2); 7.4964 (1.3); 7.4920
(1.4); 7.4895 (1.3); 7.4851 (1.2); 7.4406 (1.1); 7.4388 (1.2); 7.4286 (1.0); 7.4269 (1.1); 7.4208 (1.0); 7.4191 (1.1); 7.4089 (1.0);
7.4071 (1.0); 7.2619 (13.8); 5.3002 (7.6); 4.8736 (15.2); 4.2765 (1.9); 4.2586 (6.2); 4.2408 (6.2); 4.2230 (2.0); 1.2984 (7.8);
1.2876 (0.6); 1.2806 (16.0); 1.2628 (7.6); 0.0699 (1.0); 0.0080 (0.6); −0.0002 (20.5); −0.0085 (0.6)
I-226: ¹H-NMR(400.6 MHz, CDCl3):
δ = 8.4763 (1.6); 8.4695 (1.7); 8.2907 (1.0); 8.2870 (1.7); 8.2834 (1.0); 7.5925 (0.6); 7.5775 (0.6); 7.5715 (0.9); 7.5566 (1.0);
7.5502 (0.6); 7.5352 (0.6); 7.4157 (0.7); 7.4114 (0.7); 7.4089 (0.8); 7.4045 (0.7); 7.3938 (0.7); 7.3894 (0.8); 7.3869 (0.7); 7.3825
(0.7); 7.2621 (6.5); 6.8690 (0.6); 6.8629 (0.7); 6.8597 (0.7); 6.8535 (0.6); 6.8473 (0.6); 6.8411 (0.6); 6.8379 (0.6); 6.8318 (0.6);
6.3666 (1.0); 6.3377 (1.2); 6.3225 (1.4); 6.2936 (1.4); 6.0202 (1.5); 6.0157 (1.5); 5.9761 (1.2); 5.9716 (1.1); 5.3369 (1.4); 5.3325
(1.3); 5.3081 (1.3); 5.3034 (1.8); 5.3005 (3.1); 5.2841 (1.7); 3.7853 (16.0); 1.7040 (6.7); 1.6867 (6.6); 1.5838 (1.4); −0.0002 (8.9)
I-228: ¹H-NMR(400.6 MHz, CDCl3):
δ = 8.5127 (0.6); 8.5005 (4.8); 8.4938 (4.8); 8.3398 (3.3); 8.3365 (4.6); 8.3333 (3.3); 7.5979 (1.5); 7.5830 (1.7); 7.5765 (2.4);
7.5737 (2.1); 7.5691 (2.0); 7.5668 (2.0); 7.5623 (3.5); 7.5557 (1.8); 7.5521 (1.8); 7.5477 (1.9); 7.5453 (1.8); 7.5409 (2.7); 7.2620
(36.0); 6.8757 (1.6); 6.8695 (1.8); 6.8664 (1.8); 6.8603 (1.7); 6.8539 (1.5); 6.8478 (1.6); 6.8446 (1.6); 6.8385 (1.4); 5.3262 (1.2);
5.3088 (5.0); 5.2913 (5.1); 5.2740 (1.2); 5.1408 (1.1); 5.1371 (3.5); 5.1330 (5.3); 5.1290 (3.5); 5.1253 (0.9); 4.9934 (3.7); 4.9912
(4.7); 4.9891 (4.4); 4.9869 (3.4); 1.9639 (0.6); 1.9604 (0.8); 1.9582 (0.7); 1.9304 (11.6); 1.9281 (16.0); 1.9268 (15.8); 1.9246
(12.0); 1.7450 (0.7); 1.7275 (0.7); 1.7090 (15.3); 1.7032 (1.9); 1.6916 (15.6); 1.6858 (1.8); 1.6790 (1.6); 1.6763 (1.2); 1.6496 (0.5);
1.4321 (1.9); 0.0079 (1.3); −0.0002 (50.6); −0.0085 (1.5)
I-229: ¹H-NMR(400.6 MHz, d₆-DMSO):
δ = 12.8671 (0.8); 8.6493 (5.2); 8.6423 (5.2); 8.3809 (2.8); 8.3766 (5.1); 8.3726 (2.4); 8.1191 (1.9); 8.1171 (2.1); 8.1145 (2.5);
8.1124 (2.2); 8.1070 (2.0); 8.1050 (2.4); 8.1023 (2.3); 8.1003 (2.2); 8.0252 (1.8); 8.0205 (1.7); 8.0066 (2.2); 8.0046 (2.4); 8.0019
(2.0); 7.9999 (2.2); 7.9860 (2.4); 7.9813 (2.2); 7.8371 (1.9); 7.8328 (1.9); 7.8301 (1.9); 7.8258 (1.7); 7.8130 (2.0); 7.8088 (2.1);
7.8061 (1.9); 7.8012 (3.3); 7.7986 (4.4); 7.7964 (2.5); 7.7803 (2.1); 7.7781 (3.7); 7.7757 (2.0); 7.3260 (2.2); 7.3234 (2.3); 7.3138
(2.0); 7.3114 (2.2); 7.3075 (2.2); 7.3049 (1.9); 7.2953 (2.1); 7.2928 (2.1); 5.7569 (2.8); 4.0156 (16.0); 3.3228 (0.7); 2.5244 (1.1);
2.5197 (1.3); 2.5109 (17.5); 2.5064 (37.9); 2.5018 (52.8); 2.4972 (35.7); 2.4926 (15.4); 0.0080 (1.9); −0.0002 (70.2); −0.0085 (1.9)
I-230: ¹H-NMR(400.6 MHz, d₆-DMSO):
δ = 13.0445 (1.0); 8.6443 (5.9); 8.6373 (5.8); 8.6167 (2.2); 8.6128 (2.4); 8.6046 (2.4); 8.6007 (2.4); 8.4824 (2.5); 8.4783 (2.5);
8.4624 (2.8); 8.4582 (2.5); 8.2956 (3.0); 8.2915 (5.6); 8.2878 (2.8); 7.7884 (2.0); 7.7840 (2.1); 7.7815 (2.1); 7.7771 (1.9); 7.7650
(2.2); 7.7606 (2.3); 7.7581 (2.0); 7.7537 (1.9); 7.7371 (1.7); 7.7356 (1.7); 7.7235 (1.7); 7.7171 (1.7); 7.7051 (1.6); 5.7575 (4.6);
4.8331 (16.0); 3.3256 (8.8); 2.5255 (1.0); 2.5209 (1.4); 2.5122 (21.4); 2.5076 (46.2); 2.5030 (64.4); 2.4984 (43.6); 2.4939 (18.9);
2.0865 (1.4); 1.9894 (0.6); 1.3335 (0.9); 1.1814 (0.8); 0.0080 (1.2); −0.0002 (51.4); −0.0085 (1.4)
I-231: ¹H-NMR(400.6 MHz, d₆-DMSO):
δ = 13.1411 (2.0); 8.7009 (5.3); 8.6940 (5.4); 8.4433 (2.8); 8.4393 (5.1); 8.4357 (2.7); 8.2082 (1.2); 8.1928 (1.3); 8.1861 (2.4);
8.1707 (2.4); 8.1640 (1.3); 8.1486 (1.2); 7.9413 (2.0); 7.9369 (2.0); 7.9343 (2.1); 7.9300 (1.9); 7.9176 (2.1); 7.9132 (2.3); 7.9107
(2.0); 7.9063 (1.8); 7.4136 (1.3); 7.4071 (1.8); 7.4055 (1.8); 7.3990 (1.5); 7.3914 (1.2); 7.3849 (1.6); 7.3834 (1.6); 7.3769 (1.2);
4.8906 (16.0); 3.3243 (2.5); 2.5254 (0.8); 2.5207 (1.2); 2.5119 (16.4); 2.5074 (36.2); 2.5028 (51.4); 2.4982 (36.2); 2.4936 (16.6);
1.7598 (0.6); 1.3562 (3.2); 0.0080 (1.8); 0.0048 (0.5); 0.0039 (0.7); −0.0002 (66.0); −0.0058 (1.1); −0.0066 (0.9); −0.0085 (2.1)
I-232: ¹H-NMR(400.6 MHz, CDCl3):
δ = 8.4337 (1.0); 8.4269 (1.1); 8.2766 (1.2); 8.1658 (0.7); 8.1640 (0.8); 8.1622 (0.8); 8.1605 (0.8); 8.1542 (0.7); 8.1523 (0.9);
8.1506 (0.8); 8.1488 (0.8); 7.4930 (0.6); 7.4894 (0.7); 7.4725 (0.8); 7.4691 (1.2); 7.4658 (0.7); 7.4488 (0.8); 7.4452 (0.8); 7.3786
(0.7); 7.3743 (0.7); 7.3718 (0.7); 7.3674 (0.7); 7.3564 (0.7); 7.3521 (0.7); 7.3495 (0.7); 7.3452 (0.7); 7.2864 (0.7); 7.2774 (0.8);
7.2747 (0.8); 7.2658 (1.4); 7.2619 (8.4); 7.2570 (0.8); 7.2541 (0.7); 7.2452 (0.6); 6.4062 (1.0); 6.3774 (1.2); 6.3621 (1.4); 6.3332
(1.3); 6.0239 (1.4); 6.0193 (1.5); 5.9798 (1.2); 5.9752 (1.2); 5.3378 (1.5); 5.3271 (1.5); 5.3224 (1.6); 5.3206 (1.8); 5.2983 (1.3);
5.2936 (1.3); 3.7751 (16.0); 3.7677 (0.8); 1.7026 (6.5); 1.6852 (6.5); 1.5763 (4.1); −0.0002 (12.6)
I-233: ¹H-NMR(400.6 MHz, CDCl3):
δ = 8.4005 (1.7); 8.3936 (1.7); 8.2530 (1.0); 8.2491 (1.7); 8.2454 (1.0); 8.2159 (0.7); 8.2145 (0.7); 8.2115 (0.8); 8.2101 (0.7);
8.2040 (0.7); 8.2027 (0.8); 8.1996 (0.8); 8.1982 (0.7); 7.6585 (0.7); 7.6567 (0.7); 7.6541 (0.8); 7.6522 (0.6); 7.6394 (0.7); 7.6376
(0.8); 7.6350 (0.8); 7.6332 (0.7); 7.4986 (0.8); 7.4942 (0.8); 7.4917 (0.8); 7.4873 (0.7); 7.4759 (0.8); 7.4715 (0.8); 7.4690 (0.8);
7.4646 (0.7); 7.2623 (8.2); 7.2519 (1.0); 7.2401 (1.0); 7.2329 (0.9); 7.2210 (0.9); 5.3001 (2.4); 4.9180 (8.4); 3.7921 (16.0); 2.2181
(8.4); 1.5924 (1.4); −0.0002 (10.6)
I-236: ¹H-NMR(400.6 MHz, d₆-DMSO):
δ = 13.1306 (0.5); 8.4323 (3.6); 8.4305 (3.8); 8.4261 (3.9); 8.4243 (3.6); 8.1361 (1.6); 8.1340 (1.8); 8.1314 (2.0); 8.1293 (1.8);
8.1240 (1.8); 8.1219 (2.0); 8.1193 (2.0); 8.1172 (1.8); 8.0051 (1.6); 8.0003 (1.5); 7.9865 (1.8); 7.9845 (1.9); 7.9818 (1.7); 7.9797
(1.8); 7.9659 (1.9); 7.9612 (1.8); 7.9053 (3.4); 7.8991 (3.3); 7.8845 (4.0); 7.8784 (4.0); 7.6972 (2.0); 7.6949 (3.6); 7.6927 (2.0);
7.6766 (1.8); 7.6744 (3.2); 7.6721 (1.7); 7.6371 (4.3); 7.6353 (4.2); 7.6164 (3.8); 7.6146 (3.8); 7.3102 (1.9); 7.3078 (1.9); 7.2981
(1.7); 7.2956 (1.8); 7.2917 (1.8); 7.2892 (1.7); 7.2796 (1.8); 7.2771 (1.7); 4.9366 (12.6); 3.6216 (1.0); 3.6177 (5.5); 3.6155 (3.2);
3.6115 (3.0); 3.6093 (2.3); 3.6074 (3.9); 3.6028 (4.4); 3.6011 (12.7); 3.5989 (4.5); 3.5950 (4.1); 3.5930 (2.3); 3.5908 (2.9); 3.5867
(3.0); 3.5845 (5.7); 3.5806 (1.0); 3.3191 (12.4); 2.6701 (0.6); 2.5239 (1.7); 2.5192 (2.4); 2.5105 (31.1); 2.5059 (68.0); 2.5013
(94.3); 2.4966 (65.6); 2.4920 (28.7); 2.3283 (0.6); 1.9086 (2.7); 1.7808 (1.0); 1.7762 (5.6); 1.7685 (4.6); 1.7671 (3.5); 1.7641 (2.8);
1.7596 (16.0); 1.7548 (2.9); 1.7507 (4.4); 1.7430 (5.3); 1.7384 (0.8); 1.3553 (3.6); 0.0080 (2.2); −0.0002 (79.5); −0.0085 (2.2)
I-237: ¹H-NMR(400.6 MHz, CDCl3):
δ = 8.3594 (1.4); 8.3578 (1.5); 8.3534 (1.5); 8.3517 (1.5); 8.0676 (0.7); 8.0656 (0.8); 8.0630 (0.8); 8.0609 (0.8); 8.0555 (0.8);
8.0535 (0.8); 8.0508 (0.8); 8.0488 (0.8); 7.7733 (0.6); 7.7686 (0.6); 7.7548 (0.7); 7.7527 (0.9); 7.7502 (0.7); 7.7480 (0.8); 7.7342
(0.9); 7.7295 (0.8); 7.7228 (1.4); 7.7167 (1.4); 7.7022 (1.6); 7.6961 (1.6); 7.6330 (1.0); 7.6307 (1.7); 7.6285 (1.0); 7.6123 (0.7);
7.6100 (1.3); 7.6078 (0.7); 7.3871 (1.7); 7.3854 (1.9); 7.3665 (1.6); 7.3648 (1.7); 7.2619 (5.7); 7.1128 (0.8); 7.1103 (0.8); 7.1007
(0.8); 7.0981 (0.8); 7.0945 (0.8); 7.0919 (0.8); 7.0823 (0.8); 7.0797 (0.7); 5.2996 (2.7); 5.2719 (1.6); 5.2545 (1.6); 3.7988 (16.0);
3.7227 (0.9); 1.7185 (6.3); 1.7011 (6.2); 1.5692 (1.2); −0.0002 (8.4)
I-238: ¹H-NMR(400.6 MHz, d₆-DMSO):
δ = 13.0765 (0.7); 8.4267 (6.0); 8.4249 (6.5); 8.4205 (6.6); 8.4187 (6.1); 8.1293 (2.8); 8.1273 (3.0); 8.1247 (3.4); 8.1226 (3.0);
8.1173 (3.0); 8.1152 (3.3); 8.1126 (3.2); 8.1105 (2.9); 8.0061 (2.6); 8.0014 (2.4); 7.9876 (3.1); 7.9855 (3.1); 7.9829 (2.8); 7.9808
(3.0); 7.9670 (3.3); 7.9622 (3.0); 7.9024 (5.8); 7.8962 (5.4); 7.8817 (6.8); 7.8754 (6.8); 7.6686 (3.3); 7.6663 (6.0); 7.6640 (3.3);
7.6479 (3.1); 7.6457 (5.5); 7.6434 (3.0); 7.6349 (7.3); 7.6330 (7.1); 7.6142 (6.4); 7.6123 (6.4); 7.3040 (3.1); 7.3016 (3.1); 7.2919
(2.8); 7.2894 (3.0); 7.2855 (3.1); 7.2830 (2.7); 7.2734 (3.0); 7.2709 (2.8); 5.1820 (1.2); 5.1645 (6.2); 5.1471 (6.3); 5.1298 (1.2);
4.0555 (0.6); 4.0377 (1.9); 4.0200 (2.0); 4.0022 (0.6); 3.3176 (46.1); 2.6791 (0.8); 2.6744 (1.5); 2.6697 (2.1); 2.6651 (1.4); 2.6603
(0.7); 2.5507 (0.8); 2.5459 (0.9); 2.5404 (1.8); 2.5235 (4.8); 2.5188 (7.6); 2.5101 (111.1); 2.5055 (244.9); 2.5009 (341.1); 2.4963
(235.9); 2.4917 (103.2); 2.3371 (0.7); 2.3327 (1.4); 2.3280 (1.9); 2.3233 (1.4); 2.3187 (0.6); 1.9883 (9.1); 1.9082 (5.4); 1.6033
(16.0); 1.5859 (15.9); 1.3550 (3.5); 1.2382 (1.7); 1.2211 (1.3); 1.1922 (2.8); 1.1744 (5.8); 1.1566 (2.7); 0.1458 (0.8); 0.0079 (7.7);
0.0063 (1.9); 0.0055 (2.1); 0.0046 (2.7); 0.0038 (3.9); −0.0002 (281.4); −0.0027 (11.1); −0.0043 (4.3); −0.0052 (3.2); −0.0060
(2.7); −0.0068 (2.3); −0.0085 (8.2); −0.0108 (1.4); −0.0115 (1.2); −0.0123 (1.0); −0.0132 (0.8); −0.0139 (0.8); −0.0147 (0.8); −0.0155
(0.6); −0.0164 (0.6); −0.0171 (0.6); −0.0179 (0.6); −0.0187 (0.5); −0.0195 (0.6); −0.0211 (0.5); −0.0266 (0.7); −0.0275 (0.8); −0.0307
(0.6); −0.1494 (0.7)
I-239: ¹H-NMR(400.6 MHz, CDCl3):
δ = 8.3563 (2.8); 8.3547 (2.7); 8.3502 (3.0); 8.3486 (2.6); 8.0794 (1.4); 8.0774 (1.5); 8.0747 (1.6); 8.0727 (1.4); 8.0673 (1.4);
8.0652 (1.5); 8.0626 (1.6); 8.0606 (1.4); 7.7750 (1.1); 7.7702 (1.1); 7.7565 (1.4); 7.7543 (1.7); 7.7518 (1.4); 7.7496 (1.6); 7.7360
(1.7); 7.7312 (1.6); 7.7224 (2.6); 7.7163 (2.6); 7.7018 (2.9); 7.6957 (2.9); 7.6520 (1.9); 7.6498 (3.2); 7.6475 (1.8); 7.6313 (1.4);
7.6292 (2.2); 7.6269 (1.2); 7.3886 (3.5); 7.3870 (3.3); 7.3681 (3.2); 7.3664 (2.9); 7.2619 (10.0); 7.1215 (1.5); 7.1189 (1.4); 7.1093
(1.4); 7.1068 (1.5); 7.1031 (1.5); 7.1005 (1.4); 7.0910 (1.4); 7.0884 (1.3); 5.2997 (6.1); 4.9426 (16.0); 4.3207 (2.0); 4.3029 (6.4);
4.2851 (6.4); 4.2673 (2.1); 1.5677 (1.1); 1.3316 (7.6); 1.3138 (15.7); 1.2960 (7.5); −0.0002 (15.0)
I-240: ¹H-NMR(400.0 MHz, d₆-DMSO):
δ = 8.4105 (3.3); 8.4056 (3.4); 8.1171 (1.7); 8.1143 (1.9); 8.1049 (1.8); 8.1022 (1.8); 7.9997 (1.2); 7.9950 (1.2); 7.9791 (1.8);
7.9763 (1.6); 7.9605 (1.5); 7.9558 (1.4); 7.8885 (2.5); 7.8823 (2.4); 7.8678 (2.9); 7.8616 (2.9); 7.6645 (3.1); 7.6439 (2.8); 7.6297
(3.7); 7.6090 (3.2); 7.2948 (1.6); 7.2927 (1.5); 7.2827 (1.5); 7.2805 (1.6); 7.2764 (1.6); 7.2742 (1.4); 7.2642 (1.5); 7.2620 (1.4);
5.1700 (0.7); 5.1527 (3.0); 5.1352 (3.1); 5.1180 (0.7); 4.0380 (1.5); 4.0202 (1.6); 4.0023 (0.5); 3.3220 (16.0); 2.5240 (1.1); 2.5192
(1.7); 2.5105 (25.3); 2.5060 (54.3); 2.5015 (75.0); 2.4969 (53.5); 2.4925 (24.7); 1.9886 (6.8); 1.9088 (8.9); 1.5992 (9.0); 1.5818
(9.0); 1.3554 (1.4); 1.2389 (0.7); 1.2218 (0.6); 1.1923 (1.8); 1.1745 (3.6); 1.1567 (1.8); 0.0080 (1.3); −0.0002 (40.9); −0.0085 (1.4)
I-241: ¹H-NMR(400.6 MHz, CDCl3):
δ = 8.3593 (3.0); 8.3543 (2.6); 8.3532 (3.0); 8.0868 (1.5); 8.0840 (1.5); 8.0822 (1.5); 8.0763 (1.3); 8.0747 (1.5); 8.0719 (1.5);
8.0701 (1.4); 7.7795 (1.0); 7.7748 (1.0); 7.7609 (1.2); 7.7590 (1.6); 7.7566 (1.3); 7.7543 (1.6); 7.7404 (1.5); 7.7357 (1.5); 7.7264
(2.4); 7.7203 (2.3); 7.7058 (2.7); 7.6997 (2.6); 7.6491 (3.1); 7.6284 (2.2); 7.3904 (3.0); 7.3891 (3.6); 7.3698 (2.7); 7.3685 (3.2);
7.2618 (9.2); 7.1267 (1.3); 7.1243 (1.4); 7.1145 (1.3); 7.1122 (1.4); 7.1084 (1.4); 7.1059 (1.3); 7.0962 (1.3); 7.0938 (1.2); 5.2997
(1.4); 4.9436 (16.0); 4.3226 (2.0); 4.3048 (6.2); 4.2870 (6.3); 4.2692 (2.1); 1.5674 (1.7); 1.3327 (7.2); 1.3149 (14.4); 1.2971
(7.0); −0.0002 (13.3)
I-242: ¹H-NMR(400.6 MHz, CDCl3):
δ = 8.3539 (1.5); 8.3522 (1.5); 8.3479 (1.6); 8.3460 (1.4); 8.0582 (0.7); 8.0561 (0.8); 8.0535 (0.8); 8.0513 (0.7); 8.0460 (0.7);
8.0439 (0.8); 8.0413 (0.8); 8.0392 (0.7); 7.7688 (0.6); 7.7641 (0.6); 7.7504 (0.7); 7.7481 (0.9); 7.7457 (0.7); 7.7434 (0.9); 7.7298
(0.9); 7.7250 (0.9); 7.7181 (1.5); 7.7120 (1.4); 7.6975 (1.7); 7.6914 (1.6); 7.6340 (1.0); 7.6316 (1.7); 7.6293 (1.0); 7.6133 (0.7);
7.6110 (1.2); 7.6087 (0.7); 7.3848 (1.8); 7.3830 (1.9); 7.3643 (1.7); 7.3624 (1.7); 7.2618 (6.6); 7.1073 (0.8); 7.1048 (0.8); 7.0952
(0.8); 7.0926 (0.8); 7.0890 (0.8); 7.0863 (0.8); 7.0768 (0.8); 7.0742 (0.7); 5.2996 (2.2); 5.2668 (1.5); 5.2494 (1.6); 3.7973 (16.0);
3.7242 (0.7); 3.7218 (0.8); 1.7175 (6.1); 1.7001 (6.0); 1.5682 (0.7); −0.0002 (10.2)
I-243: ¹H-NMR(400.6 MHz, CDCl3):
δ = 8.3671 (1.9); 8.3610 (1.9); 8.0880 (0.9); 8.0860 (0.9); 8.0834 (1.0); 8.0759 (1.0); 8.0739 (1.0); 8.0713 (1.0); 7.7927 (0.6);
7.7880 (0.6); 7.7741 (0.9); 7.7720 (1.0); 7.7695 (0.9); 7.7675 (0.8); 7.7537 (0.9); 7.7490 (0.8); 7.7300 (1.4); 7.7239 (1.4); 7.7093
(1.6); 7.7033 (1.5); 7.6671 (1.4); 7.6651 (1.9); 7.6465 (1.0); 7.6445 (1.3); 7.3910 (2.2); 7.3704 (2.0); 7.2617 (7.9); 7.1311 (0.9);
7.1288 (0.8); 7.1189 (0.9); 7.1165 (0.8); 7.1128 (0.9); 7.1104 (0.7); 7.1006 (0.8); 7.0983 (0.7); 4.9461 (8.9); 4.5224 (2.2); 4.5067
(4.5); 4.4911 (2.2); 3.6827 (16.0); 2.7174 (2.2); 2.7017 (4.4); 2.6860 (2.1); 1.5591 (2.6); −0.0002 (11.3)
I-244: ¹H-NMR(400.6 MHz, CDCl3):
δ = 8.3651 (1.6); 8.3634 (1.7); 8.3590 (1.6); 8.3574 (1.7); 8.0547 (0.7); 8.0527 (0.8); 8.0500 (0.9); 8.0480 (0.8); 8.0426 (0.8);
8.0406 (0.9); 8.0379 (0.9); 8.0359 (0.8); 7.7830 (0.6); 7.7783 (0.6); 7.7646 (0.7); 7.7623 (1.0); 7.7599 (0.8); 7.7576 (0.9); 7.7440
(0.9); 7.7392 (0.9); 7.7259 (1.5); 7.7198 (1.4); 7.7053 (1.6); 7.6992 (1.7); 7.6649 (1.0); 7.6626 (1.8); 7.6604 (1.1); 7.6443 (0.8);
7.6420 (1.2); 7.6397 (0.8); 7.3845 (1.9); 7.3828 (2.0); 7.3639 (1.7); 7.3622 (1.8); 7.2611 (13.5); 7.1078 (0.8); 7.1052 (0.8); 7.0957
(0.8); 7.0931 (0.8); 7.0895 (0.8); 7.0869 (0.8); 7.0774 (0.8); 7.0748 (0.8); 5.2290 (1.7); 5.2116 (1.7); 4.4870 (1.0); 4.4800 (1.0);
4.4713 (2.1); 4.4645 (2.1); 4.4557 (1.1); 4.4488 (1.0); 3.6540 (16.0); 2.6865 (1.6); 2.6706 (2.6); 2.6551 (1.4); 1.7035 (6.1); 1.6860
(6.1); 1.5518 (3.7); −0.0002 (19.6); −0.0085 (0.6)
I-245: ¹H-NMR(400.6 MHz, CDCl3):
δ = 8.3702 (1.7); 8.3685 (1.6); 8.3641 (1.8); 8.3624 (1.5); 8.0640 (0.8); 8.0620 (0.9); 8.0593 (0.9); 8.0573 (0.8); 8.0519 (0.9);
8.0498 (0.9); 8.0472 (0.9); 8.0451 (0.8); 7.7870 (0.6); 7.7823 (0.6); 7.7685 (0.8); 7.7663 (1.0); 7.7638 (0.8); 7.7616 (0.8); 7.7479
(1.0); 7.7432 (0.9); 7.7301 (1.5); 7.7240 (1.5); 7.7095 (1.6); 7.7034 (1.7); 7.6635 (1.2); 7.6613 (1.8); 7.6590 (1.0); 7.6429 (0.9);
7.6407 (1.3); 7.6383 (0.6); 7.3868 (2.1); 7.3851 (1.8); 7.3662 (1.9); 7.3645 (1.6); 7.2611 (13.9); 7.1132 (0.9); 7.1106 (0.8); 7.1010
(0.9); 7.0984 (0.8); 7.0948 (0.9); 7.0923 (0.7); 7.0827 (0.8); 7.0801 (0.7); 5.3000 (2.1); 5.2327 (1.7); 5.2153 (1.7); 4.4877 (1.1);
4.4825 (1.1); 4.4719 (2.2); 4.4670 (2.1); 4.4563 (1.2); 4.4513 (1.0); 3.6533 (16.0); 2.6881 (1.6); 2.6727 (2.7); 2.6570 (1.4); 1.7043
(6.1); 1.6869 (6.0); 1.5511 (4.6); 0.0080 (0.7); −0.0002 (20.1); −0.0085 (0.6)
I-246: ¹H-NMR(400.6 MHz, CDCl3):
δ = 8.3631 (1.8); 8.3615 (1.7); 8.3570 (1.8); 8.3555 (1.6); 8.0790 (0.9); 8.0771 (1.0); 8.0744 (1.0); 8.0725 (0.8); 8.0669 (0.9);
8.0649 (1.0); 8.0622 (1.0); 8.0603 (0.8); 7.7885 (0.6); 7.7838 (0.6); 7.7701 (0.9); 7.7679 (1.1); 7.7655 (0.9); 7.7633 (0.9); 7.7495
(1.0); 7.7448 (0.9); 7.7262 (1.5); 7.7201 (1.4); 7.7056 (1.6); 7.6995 (1.6); 7.6681 (1.3); 7.6659 (1.9); 7.6475 (1.0); 7.6453 (1.3);
7.3890 (2.1); 7.3874 (1.9); 7.3684 (1.9); 7.3668 (1.7); 7.2614 (9.9); 7.1259 (0.9); 7.1234 (0.8); 7.1138 (0.9); 7.1112 (0.9); 7.1076
(0.9); 7.1051 (0.8); 7.0955 (0.8); 7.0929 (0.7); 5.2999 (1.4); 4.9444 (8.6); 4.5203 (2.1); 4.5046 (4.4); 4.4889 (2.2); 3.6823 (16.0);
2.7157 (2.1); 2.7000 (4.2); 2.6843 (2.0); 1.5553 (3.3); −0.0002 (14.8)
I-248: ¹H-NMR(400.6 MHz, CDCl3):
δ = 8.1714 (1.6); 8.1695 (1.8); 8.1675 (1.6); 8.1653 (1.9); 8.1634 (1.7); 8.0500 (1.2); 8.0478 (1.3); 8.0453 (1.5); 8.0432 (1.3);
8.0378 (1.3); 8.0356 (1.3); 8.0333 (1.5); 8.0310 (1.3); 7.8168 (0.9); 7.8106 (0.9); 7.7976 (1.1); 7.7958 (1.2); 7.7914 (1.1); 7.7897
(1.1); 7.7767 (1.0); 7.7705 (1.0); 7.7525 (0.6); 7.7478 (0.5); 7.7346 (1.5); 7.7318 (1.1); 7.7299 (1.5); 7.7271 (1.0); 7.7170 (1.1);
7.7138 (1.2); 7.7125 (1.3); 7.7092 (1.1); 7.7013 (1.0); 7.6988 (1.2); 7.6981 (1.4); 7.6956 (1.1); 7.6853 (1.0); 7.6825 (1.5); 7.6802
(1.4); 7.6785 (0.7); 7.6647 (0.5); 7.6622 (0.7); 7.2612 (22.4); 7.0817 (1.5); 7.0785 (1.4); 7.0696 (1.5); 7.0663 (1.5); 7.0639 (1.5);
7.0607 (1.4); 7.0518 (1.4); 7.0486 (1.4); 6.9879 (1.4); 6.9863 (1.5); 6.9805 (1.4); 6.9790 (1.4); 6.9668 (1.4); 6.9653 (1.4); 6.9594
(1.4); 6.9578 (1.4); 5.2999 (4.5); 5.2523 (1.5); 5.2509 (1.6); 5.2346 (1.6); 5.2333 (1.6); 5.0717 (1.0); 5.0314 (3.4); 5.0204 (7.2);
5.0060 (3.4); 4.9657 (1.0); 3.8161 (1.7); 3.7499 (16.0); 3.7450 (15.8); 2.3354 (0.6); 2.3257 (0.9); 2.3226 (0.8); 2.3060 (1.0);
2.3029 (0.9); 1.8693 (0.6); 1.8624 (0.6); 1.8496 (0.6); 1.8427 (0.6); 1.6816 (0.5); 1.5578 (1.2); 1.5526 (6.7); 1.5443 (6.6); 1.5350
(6.3); 1.5266 (6.4); 0.0079 (0.9); −0.0002 (32.4); −0.0085 (0.9)
I-249: ¹H-NMR(400.6 MHz, d₆-DMSO):
δ = 13.0053 (1.1); 8.2439 (4.5); 8.2376 (4.6); 8.1077 (2.9); 8.1057 (3.2); 8.1030 (3.7); 8.1009 (3.3); 8.0956 (3.1); 8.0935 (3.6);
8.0909 (3.5); 8.0888 (3.2); 7.9676 (2.3); 7.9656 (3.4); 7.9610 (4.2); 7.9470 (5.9); 7.9451 (4.6); 7.9421 (5.2); 7.9403 (6.0); 7.9266
(5.3); 7.9216 (4.3); 7.6663 (3.4); 7.6640 (6.3); 7.6618 (3.6); 7.6458 (3.2); 7.6435 (5.8); 7.6412 (3.1); 7.2682 (3.1); 7.2653 (4.3);
7.2628 (6.1); 7.2532 (3.1); 7.2507 (3.8); 7.2469 (6.0); 7.2443 (3.8); 7.2414 (3.0); 7.2401 (2.8); 7.2347 (3.3); 7.2322 (3.1); 6.8712
(0.6); 4.9189 (2.0); 4.8789 (11.6); 4.8695 (11.2); 4.8295 (2.0); 3.6217 (0.8); 3.6178 (4.6); 3.6155 (2.6); 3.6115 (2.4); 3.6094 (1.8);
3.6075 (3.2); 3.6011 (11.0); 3.5990 (3.8); 3.5950 (3.4); 3.5931 (1.8); 3.5908 (2.4); 3.5867 (2.4); 3.5845 (5.0); 3.5806 (0.8); 3.3215
(16.0); 2.7481 (1.0); 2.7284 (1.2); 2.7183 (1.9); 2.6982 (2.1); 2.6880 (1.2); 2.6749 (0.6); 2.6683 (1.4); 2.5409 (3.8); 2.5287 (0.6);
2.5241 (2.1); 2.5195 (2.6); 2.5107 (37.1); 2.5061 (82.2); 2.5015 (115.5); 2.4969 (79.5); 2.4923 (34.9); 2.3286 (0.7); 2.1830 (0.9);
1.9154 (0.5); 1.9088 (2.5); 1.9038 (1.1); 1.8905 (0.9); 1.8852 (0.9); 1.8729 (1.0); 1.8608 (0.6); 1.7807 (0.7); 1.7761 (4.8); 1.7745
(2.0); 1.7685 (3.7); 1.7670 (2.9); 1.7641 (2.2); 1.7595 (13.4); 1.7548 (2.3); 1.7506 (3.6); 1.7430 (4.5); 1.7384 (0.7); 1.6103 (0.6);
1.6008 (0.8); 1.5903 (1.2); 1.5783 (0.8); 1.5694 (1.1); 1.5584 (0.9); 1.3556 (8.6); 0.0271 (0.6); 0.0112 (0.5); 0.0104 (0.5); 0.0095
(0.6); 0.0080 (3.5); 0.0065 (1.0); 0.0056 (1.1); 0.0048 (1.3); 0.0040 (1.8); 0.0031 (2.8); 0.0023 (4.6); −0.0002 (108.5); −0.0041
(1.2); −0.0050 (0.7); −0.0058 (0.5); −0.0085 (2.6)
I-250: ¹H-NMR(400.6 MHz, CDCl3):
δ = 8.2620 (1.3); 8.2601 (1.6); 8.2584 (1.7); 8.2566 (1.5); 8.2504 (1.4); 8.2485 (1.7); 8.2468 (1.7); 8.2450 (1.5); 8.1437 (2.1);
8.1375 (2.3); 7.7569 (1.0); 7.7506 (1.0); 7.7379 (1.2); 7.7358 (1.4); 7.7317 (1.3); 7.7296 (1.4); 7.7168 (1.1); 7.7106 (1.1); 7.4876
(1.1); 7.4839 (1.2); 7.4669 (1.5); 7.4634 (1.9); 7.4607 (1.4); 7.4438 (1.5); 7.4400 (1.4); 7.3264 (1.4); 7.3174 (1.6); 7.3148 (1.5);
7.3058 (2.5); 7.2968 (1.1); 7.2942 (1.2); 7.2851 (1.0); 7.2641 (16.7); 7.1188 (0.7); 6.9260 (1.4); 6.9245 (1.5); 6.9185 (1.5); 6.9171
(1.5); 6.9049 (1.4); 6.9034 (1.5); 6.8974 (1.4); 6.8959 (1.5); 5.3009 (2.3); 4.8100 (12.6); 4.1895 (1.9); 4.1716 (6.2); 4.1538 (6.3);
4.1360 (2.0); 3.6714 (1.2); 3.6560 (2.9); 3.6413 (3.0); 3.6259 (1.3); 2.6032 (2.8); 2.5879 (4.0); 2.5733 (2.7); 2.0456 (1.0); 1.6296
(1.0); 1.6216 (1.0); 1.6164 (0.8); 1.6080 (5.3); 1.6010 (8.3); 1.5876 (1.2); 1.5745 (0.6); 1.2861 (7.6); 1.2775 (0.6); 1.2683 (16.0);
1.2597 (1.0); 1.2561 (0.5); 1.2505 (7.5); 0.8518 (1.1); 0.8409 (2.4); 0.8360 (2.8); 0.8309 (1.4); 0.8253 (1.5); 0.8197 (2.8); 0.8149
(2.5); 0.8044 (1.3); 0.6162 (1.3); 0.6055 (2.7); 0.6008 (2.9); 0.5925 (2.6); 0.5878 (2.9); 0.5767 (1.0); 0.0080 (0.6); −0.0002
(23.9); −0.0084 (0.8)
I-251: ¹H-NMR(400.6 MHz, CDCl3):
δ = 8.2284 (1.6); 8.2265 (1.9); 8.2248 (1.8); 8.2168 (1.7); 8.2149 (1.9); 8.2132 (1.8); 8.1313 (2.5); 8.1251 (2.5); 7.7697 (1.1);
7.7635 (1.1); 7.7505 (1.5); 7.7486 (1.4); 7.7443 (1.4); 7.7424 (1.4); 7.7296 (1.2); 7.7234 (1.2); 7.4591 (1.2); 7.4554 (1.1); 7.4384
(1.7); 7.4354 (2.2); 7.4320 (1.3); 7.4151 (1.5); 7.4114 (1.4); 7.2847 (1.4); 7.2757 (1.7); 7.2730 (1.6); 7.2613 (21.0); 7.2551 (1.3);
7.2524 (1.2); 7.2436 (1.1); 6.9201 (1.7); 6.9127 (1.8); 6.8990 (1.7); 6.8916 (1.7); 4.8932 (16.0); 4.0831 (7.4); 4.0656 (7.6); 2.2232
(1.1); 2.2044 (1.6); 2.1857 (1.2); 1.7228 (0.8); 1.7069 (1.0); 1.6906 (1.4); 1.6783 (0.8); 1.6708 (0.7); 1.6611 (0.5); 1.6119 (0.6);
1.5986 (1.3); 1.5920 (1.7); 1.5846 (1.4); 1.5780 (3.5); 1.5700 (1.6); 1.5626 (2.6); 1.5545 (7.3); 1.5465 (1.7); 1.5389 (1.1); 1.5298
(1.9); 1.5226 (1.3); 1.5138 (1.4); 1.5038 (1.0); 1.4933 (0.6); 1.4905 (0.6); 1.2523 (0.6); 1.2465 (1.1); 1.2332 (1.1); 1.2288 (1.2);
1.2148 (1.1); 1.1974 (0.9); 0.7977 (1.5); 0.7930 (4.3); 0.7898 (3.1); 0.7857 (4.5); 0.7826 (5.7); 0.7796 (5.1); 0.7751 (2.6); 0.7667
(2.6); 0.7626 (3.9); 0.7570 (1.8); 0.0079 (1.0); −0.0002 (31.9); −0.0085 (0.9)
I-253: ¹H-NMR(400.6 MHz, CDCl3):
δ = 8.6065 (1.4); 8.5669 (1.0); 8.5562 (1.0); 8.2289 (1.5); 8.2270 (1.7); 8.2252 (1.7); 8.2236 (1.4); 8.2172 (1.6); 8.2153 (1.8);
8.2136 (1.7); 8.1350 (2.4); 8.1328 (1.8); 8.1307 (2.0); 8.1289 (2.4); 7.7748 (1.1); 7.7686 (1.1); 7.7558 (1.4); 7.7538 (1.4); 7.7496
(1.4); 7.7476 (1.4); 7.7348 (1.2); 7.7286 (1.2); 7.6978 (0.8); 7.6935 (1.3); 7.6881 (0.8); 7.6782 (1.0); 7.6739 (1.5); 7.6685 (0.9);
7.4677 (1.2); 7.4640 (1.2); 7.4471 (1.7); 7.4439 (2.1); 7.4405 (1.3); 7.4237 (1.6); 7.4199 (1.4); 7.2973 (1.4); 7.2883 (1.6); 7.2857
(1.4); 7.2767 (2.5); 7.2676 (2.2); 7.2635 (38.7); 7.2586 (0.7); 7.2561 (1.6); 7.2530 (1.3); 7.2452 (1.1); 7.2332 (1.0); 6.9236 (1.7);
6.9222 (1.6); 6.9162 (1.7); 6.9147 (1.5); 6.9025 (1.6); 6.9010 (1.5); 6.8951 (1.6); 6.8936 (1.4); 5.2442 (10.7); 4.9477 (16.0);
1.5842 (0.8); 1.5787 (0.9); 1.5723 (0.6); 1.5647 (1.9); 1.5579 (0.6); 1.5516 (0.7); 1.5499 (0.8); 1.5446 (0.9); 1.5305 (0.6); 0.7688
(1.5); 0.7642 (3.0); 0.7632 (3.2); 0.7588 (2.4); 0.7481 (4.0); 0.7431 (6.8); 0.7405 (3.4); 0.7392 (3.8); 0.7335 (2.8); 0.7299 (3.9);
0.7270 (1.8); 0.7247 (1.7); 0.7220 (0.8); 0.0080 (0.7); −0.0002 (23.9); −0.0085 (0.6)
I-254: ¹H-NMR(400.6 MHz, CDCl3):
δ = 8.2700 (0.8); 8.2681 (1.0); 8.2664 (1.0); 8.2645 (0.9); 8.2584 (0.8); 8.2565 (1.0); 8.2548 (1.0); 8.2529 (0.9); 8.1492 (1.2);
8.1473 (1.0); 8.1451 (1.0); 8.1430 (1.3); 7.7664 (0.6); 7.7601 (0.6); 7.7475 (0.7); 7.7453 (0.8); 7.7413 (0.7); 7.7391 (0.8); 7.7264
(0.7); 7.7201 (0.7); 7.4901 (0.7); 7.4863 (0.7); 7.4694 (0.9); 7.4658 (1.1); 7.4632 (0.8); 7.4463 (0.9); 7.4426 (0.9); 7.3315 (0.9);
7.3224 (1.0); 7.3199 (0.9); 7.3108 (1.5); 7.3018 (0.7); 7.2992 (0.7); 7.2901 (0.6); 7.2633 (12.8); 6.9303 (0.8); 6.9288 (0.9); 6.9229
(0.9); 6.9214 (0.9); 6.9093 (0.8); 6.9077 (0.9); 6.9018 (0.8); 6.9002 (0.8); 4.8742 (7.0); 4.1735 (3.2); 4.1608 (3.2); 3.8018 (16.0);
1.6496 (0.6); 1.6417 (0.6); 1.6287 (1.2); 1.6154 (0.6); 1.6077 (0.7); 1.5849 (5.0); 0.8728 (0.7); 0.8619 (1.4); 0.8569 (1.7); 0.8518
(0.8); 0.8463 (0.9); 0.8406 (1.6); 0.8357 (1.4); 0.8253 (0.8); 0.6547 (0.8); 0.6440 (1.6); 0.6412 (1.3); 0.6392 (1.7); 0.6310 (1.5);
0.6280 (1.2); 0.6262 (1.7); 0.6152 (0.6); 0.0080 (0.6); 0.0023 (0.7); −0.0002 (18.7); −0.0084 (0.5)
I-255: ¹H-NMR(400.6 MHz, CDCl3):
δ = 8.4608 (1.2); 8.4567 (1.3); 8.4491 (1.2); 8.4448 (1.3); 8.1404 (2.0); 8.1343 (2.2); 8.1164 (1.3); 8.1125 (1.4); 8.0969 (1.4);
8.0923 (1.4); 7.7864 (1.0); 7.7803 (0.9); 7.7674 (1.1); 7.7654 (1.2); 7.7612 (1.1); 7.7592 (1.2); 7.7464 (1.0); 7.7402 (1.0); 7.4001
(1.0); 7.3898 (1.0); 7.3821 (1.0); 7.3684 (1.0); 7.2605 (88.4); 6.9133 (1.4); 6.9058 (1.4); 6.8921 (1.3); 6.8847 (1.3); 5.3002 (2.0);
4.9466 (0.7); 4.8840 (13.2); 2.1081 (1.1); 2.0462 (1.1); 1.5898 (0.7); 1.5776 (0.6); 1.5720 (1.6); 1.5657 (0.6); 1.5556 (0.8); 1.5377
(0.6); 1.4322 (1.2); 1.2597 (0.8); 1.2226 (0.8); 0.7916 (3.5); 0.7893 (3.6); 0.7870 (3.6); 0.7848 (3.9); 0.7710 (16.0); 0.0080
(2.9); −0.0002 (110.8); −0.0085 (3.2)
I-256: ¹H-NMR(400.6 MHz, CDCl3):
δ = 8.4746 (0.7); 8.4703 (0.7); 8.4627 (0.8); 8.4583 (0.8); 8.1488 (0.8); 8.1444 (0.8); 8.1286 (0.8); 8.1247 (0.9); 8.1131 (1.1);
8.1108 (0.8); 8.1087 (0.9); 8.1068 (1.2); 7.8495 (0.6); 7.8432 (0.6); 7.8307 (0.7); 7.8282 (0.7); 7.8245 (0.7); 7.8220 (0.7); 7.8095
(0.6); 7.8033 (0.6); 7.4517 (0.6); 7.4500 (0.6); 7.4397 (0.6); 7.4380 (0.6); 7.4319 (0.6); 7.4302 (0.6); 7.4199 (0.6); 7.4182 (0.5);
7.2617 (13.6); 6.9524 (0.8); 6.9509 (0.8); 6.9449 (0.8); 6.9434 (0.8); 6.9312 (0.8); 6.9297 (0.7); 6.9237 (0.8); 6.9222 (0.8); 5.1721
(1.6); 5.1546 (1.6); 4.4618 (0.6); 4.4504 (0.8); 4.4340 (1.7); 4.4253 (0.8); 4.4176 (0.8); 4.4093 (1.7); 4.3934 (0.8); 4.3816 (0.6);
3.6782 (1.1); 3.6744 (16.0); 2.6586 (1.9); 2.6425 (3.8); 2.6263 (1.8); 1.6747 (5.9); 1.6690 (0.5); 1.6573 (5.8); 1.5568 (7.1); 0.0080
(0.5); −0.0002 (20.6); −0.0085 (0.5)
I-257: ¹H-NMR(400.6 MHz, CDCl3):
δ = 8.4918 (0.7); 8.4883 (0.7); 8.4799 (0.8); 8.4762 (0.8); 8.1483 (1.1); 8.1448 (1.1); 8.1287 (1.2); 8.1250 (1.2); 8.1066 (1.3);
8.1043 (1.3); 8.1004 (1.7); 8.0982 (1.3); 8.0958 (0.8); 7.8539 (0.6); 7.8477 (0.6); 7.8353 (1.0); 7.8327 (0.8); 7.8290 (0.8); 7.8265
(0.8); 7.8230 (0.6); 7.8205 (0.5); 7.8141 (1.0); 7.8078 (0.7); 7.4625 (0.6); 7.4522 (0.6); 7.4505 (0.6); 7.4427 (0.6); 7.4399 (0.5);
7.4307 (0.7); 7.2613 (28.8); 6.9516 (1.3); 6.9442 (1.3); 6.9304 (1.3); 6.9229 (1.3); 5.2491 (1.0); 5.2409 (1.8); 5.2316 (1.0); 5.2235
(1.8); 5.2062 (0.5); 5.1907 (0.9); 5.1730 (0.9); 5.1632 (1.5); 5.1455 (1.5); 3.7562 (1.5); 3.7336 (9.4); 3.7033 (16.0); 3.6744 (0.5);
1.7657 (3.4); 1.7482 (3.7); 1.7446 (6.2); 1.7271 (5.8); 1.7212 (0.6); 1.6903 (0.6); 1.6730 (0.7); 1.5524 (14.9); 1.4981 (3.6); 1.4804
(9.9); 1.4627 (6.4); 0.0080 (1.1); −0.0002 (40.6); −0.0058 (0.7); −0.0085 (1.2)
I-258: ¹H-NMR(400.6 MHz, CDCl3):
δ = 8.4345 (1.2); 8.4306 (1.3); 8.4225 (1.3); 8.4186 (1.3); 8.1337 (2.1); 8.1274 (2.2); 8.1068 (1.4); 8.1025 (1.4); 8.0871 (1.5);
8.0827 (1.4); 7.7809 (1.0); 7.7747 (1.0); 7.7618 (1.2); 7.7599 (1.3); 7.7557 (1.2); 7.7537 (1.2); 7.7408 (1.1); 7.7346 (1.1); 7.3747
(1.0); 7.3730 (1.0); 7.3627 (1.0); 7.3610 (1.0); 7.3550 (1.0); 7.3533 (1.0); 7.3430 (1.0); 7.3413 (1.0); 7.2611 (25.0); 6.9083 (1.3);
6.9069 (1.4); 6.9009 (1.4); 6.8995 (1.4); 6.8872 (1.3); 6.8858 (1.4); 6.8798 (1.4); 6.8784 (1.3); 4.8771 (0.8); 4.8219 (14.7); 4.2614
(2.1); 4.2436 (6.1); 4.2258 (6.0); 4.2079 (1.9); 3.9304 (0.5); 1.5934 (0.9); 1.5860 (1.0); 1.5767 (0.7); 1.5728 (1.6); 1.5647 (0.6);
1.5591 (1.0); 1.5523 (16.0); 1.5384 (0.6); 1.2988 (0.7); 1.2950 (7.4); 1.2872 (0.6); 1.2810 (1.4); 1.2771 (15.4); 1.2632 (1.6);
1.2593 (7.9); 0.8988 (0.6); 0.8819 (2.0); 0.8642 (0.8); 0.8425 (0.6); 0.8284 (2.1); 0.8232 (2.1); 0.8160 (3.2); 0.8099 (2.1); 0.8024
(2.0); 0.7936 (2.0); 0.7918 (1.4); 0.7863 (2.0); 0.7817 (1.4); 0.7731 (2.1); 0.7659 (2.2); 0.7603 (1.7); 0.7468 (0.8); 0.0080
(0.9); −0.0002 (35.2); −0.0085 (1.0)
I-259: ¹H-NMR(400.6 MHz, CDCl3):
δ = 8.2694 (1.2); 8.2675 (1.4); 8.2658 (1.4); 8.2641 (1.2); 8.2577 (1.2); 8.2559 (1.4); 8.2541 (1.4); 8.2525 (1.1); 8.1489 (1.9);
8.1468 (1.5); 8.1446 (1.5); 8.1427 (2.0); 7.7658 (0.9); 7.7596 (0.9); 7.7469 (1.1); 7.7447 (1.2); 7.7407 (1.1); 7.7385 (1.1); 7.7258
(1.0); 7.7196 (1.0); 7.4888 (1.0); 7.4851 (1.0); 7.4682 (1.4); 7.4647 (1.6); 7.4619 (1.1); 7.4450 (1.3); 7.4413 (1.3); 7.3301 (1.2);
7.3210 (1.3); 7.3185 (1.2); 7.3094 (2.0); 7.3004 (0.9); 7.2979 (0.9); 7.2888 (0.9); 7.2643 (31.5); 7.0965 (0.6); 6.9294 (1.2); 6.9280
(1.3); 6.9220 (1.3); 6.9206 (1.2); 6.9083 (1.2); 6.9068 (1.2); 6.9008 (1.3); 6.8994 (1.2); 4.8727 (10.6); 4.2865 (1.7); 4.2687 (5.6);
4.2508 (5.6); 4.2330 (1.8); 4.1545 (5.0); 4.1420 (5.0); 2.0454 (1.6); 1.6489 (0.9); 1.6410 (1.0); 1.6358 (0.6); 1.6280 (2.0); 1.6200
(1.0); 1.6147 (1.8); 1.6070 (1.6); 1.5939 (0.7); 1.3277 (7.6); 1.3099 (16.0); 1.2920 (7.5); 1.2774 (0.6); 1.2595 (1.1); 0.8713 (1.1);
0.8605 (2.3); 0.8555 (2.6); 0.8503 (1.2); 0.8448 (1.4); 0.8392 (2.5); 0.8343 (2.1); 0.8239 (1.2); 0.6563 (1.3); 0.6456 (2.5); 0.6428
(2.0); 0.6409 (2.5); 0.6326 (2.3); 0.6279 (2.5); 0.6167 (0.9); 0.0080 (0.6); −0.0002 (19.8); −0.0085 (0.5)
I-261: ¹H-NMR(400.6 MHz, CDCl3):
δ = 8.1763 (0.7); 8.1744 (1.1); 8.1724 (0.9); 8.1704 (0.9); 8.1682 (1.1); 8.1662 (0.8); 8.0545 (0.7); 8.0524 (0.8); 8.0497 (0.8);
8.0476 (0.8); 8.0423 (0.8); 8.0402 (0.8); 8.0376 (0.8); 8.0355 (0.8); 7.8185 (0.6); 7.8123 (0.6); 7.7995 (0.7); 7.7975 (0.8); 7.7934
(0.7); 7.7913 (0.7); 7.7785 (0.6); 7.7723 (0.6); 7.7543 (0.6); 7.7495 (0.6); 7.7359 (0.7); 7.7336 (1.0); 7.7312 (0.7); 7.7289 (1.0);
7.7153 (1.0); 7.7105 (1.0); 7.6411 (1.0); 7.6386 (1.7); 7.6363 (1.1); 7.6204 (0.7); 7.6181 (1.2); 7.6157 (0.7); 7.2610 (18.6); 7.0848
(0.8); 7.0822 (0.8); 7.0727 (0.8); 7.0700 (0.8); 7.0665 (0.8); 7.0638 (0.8); 7.0544 (0.8); 7.0517 (0.8); 6.9887 (0.8); 6.9871 (0.8);
6.9813 (0.8); 6.9798 (0.8); 6.9676 (0.7); 6.9660 (0.8); 6.9602 (0.8); 6.9587 (0.7); 5.3001 (0.9); 4.9226 (3.8); 4.9153 (3.8); 4.5089
(1.8); 4.4932 (4.0); 4.4776 (2.0); 3.6913 (16.0); 2.7127 (1.9); 2.6971 (3.7); 2.6814 (1.8); 2.3256 (0.9); 2.3059 (0.9); 1.8381 (0.5);
1.8183 (0.6); 1.5511 (2.7); 0.0080 (0.8); −0.0002 (28.8); −0.0085 (0.8)
I-263: ¹H-NMR(400.6 MHz, CDCl3):
δ = 8.2909 (1.8); 8.2891 (2.2); 8.2872 (2.3); 8.2856 (2.1); 8.2792 (2.0); 8.2775 (2.3); 8.2755 (2.3); 8.2740 (2.0); 8.1031 (3.0);
8.0969 (3.1); 7.7738 (1.3); 7.7676 (1.3); 7.7550 (1.5); 7.7527 (1.7); 7.7488 (1.5); 7.7464 (1.6); 7.7340 (1.4); 7.7277 (1.4); 7.4886
(1.4); 7.4849 (1.5); 7.4679 (2.0); 7.4643 (2.4); 7.4619 (1.8); 7.4448 (1.9); 7.4411 (1.9); 7.3470 (1.9); 7.3379 (2.2); 7.3354 (2.0);
7.3263 (3.3); 7.3172 (1.5); 7.3147 (1.5); 7.3056 (1.4); 7.2619 (18.8); 6.9569 (1.8); 6.9555 (2.0); 6.9496 (2.0); 6.9482 (2.0); 6.9358
(1.8); 6.9343 (1.9); 6.9284 (1.8); 6.9270 (1.8); 5.0301 (0.7); 4.9967 (0.7); 4.9694 (16.0); 2.4525 (0.9); 2.4327 (1.0); 2.4233 (1.9);
2.4035 (2.0); 2.3939 (1.1); 2.3742 (1.0); 1.8223 (0.8); 1.8113 (1.1); 1.8022 (0.6); 1.7919 (1.3); 1.7806 (1.1); 1.7704 (0.8); 1.7609
(0.7); 1.7533 (1.0); 1.7402 (1.0); 1.7337 (0.6); 1.7236 (0.6); 1.7206 (0.6); 1.7106 (0.6); 1.4321 (4.4); 0.8818 (0.7); 0.0080
(0.7); −0.0002 (24.3); −0.0085 (0.6)
I-264: ¹H-NMR(400.6 MHz, CDCl3):
δ = 8.2284 (1.9); 8.2168 (1.9); 8.1346 (2.6); 8.1285 (2.6); 7.7697 (1.1); 7.7635 (1.1); 7.7487 (1.5); 7.7425 (1.4); 7.7296 (1.2);
7.7234 (1.2); 7.4706 (1.2); 7.4669 (1.2); 7.4500 (1.6); 7.4468 (2.3); 7.4435 (1.3); 7.4266 (1.5); 7.4229 (1.4); 7.2946 (1.3); 7.2855
(1.7); 7.2829 (1.6); 7.2739 (2.6); 7.2611 (29.9); 7.2535 (1.1); 6.9196 (1.7); 6.9130 (1.7); 6.8992 (1.6); 6.8919 (1.6); 4.9088 (16.0);
4.7631 (4.4); 4.7474 (5.2); 4.7435 (4.9); 4.7277 (4.9); 4.4560 (4.2); 4.4406 (7.8); 4.4249 (4.7); 4.4214 (7.6); 4.4046 (7.5); 3.3342
(0.8); 3.3308 (0.9); 3.3147 (1.4); 3.2983 (0.9); 1.5960 (0.8); 1.5928 (0.8); 1.5846 (0.6); 1.5778 (2.0); 1.5708 (0.8); 1.5546 (7.8);
1.5436 (0.8); 0.8821 (0.8); 0.7811 (4.4); 0.7779 (3.3); 0.7740 (3.3); 0.7711 (4.4); 0.7656 (2.7); 0.7590 (10.5); 0.0079 (1.6); −0.0002
(41.4); −0.0084 (1.1)
I-265: ¹H-NMR(400.6 MHz, CDCl3):
δ = 8.2312 (1.3); 8.2293 (1.5); 8.2276 (1.5); 8.2258 (1.4); 8.2196 (1.4); 8.2177 (1.5); 8.2159 (1.5); 8.2142 (1.4); 8.0739 (2.0);
8.0677 (2.0); 7.7515 (1.0); 7.7453 (0.9); 7.7324 (1.1); 7.7304 (1.2); 7.7263 (1.2); 7.7242 (1.2); 7.7114 (1.0); 7.7052 (1.0); 7.4688
(1.1); 7.4651 (1.2); 7.4482 (1.5); 7.4445 (1.7); 7.4420 (1.3); 7.4251 (1.4); 7.4213 (1.4); 7.3037 (1.3); 7.2947 (1.4); 7.2921 (1.4);
7.2830 (2.3); 7.2741 (1.1); 7.2714 (1.2); 7.2613 (38.0); 6.8867 (1.4); 6.8852 (1.5); 6.8793 (1.5); 6.8778 (1.4); 6.8656 (1.4); 6.8641
(1.4); 6.8582 (1.4); 6.8567 (1.3); 5.1293 (0.6); 5.1255 (2.0); 5.1212 (2.8); 5.1170 (2.2); 5.1132 (0.6); 5.0671 (2.3); 5.0648 (2.8);
5.0624 (2.6); 5.0601 (2.0); 4.9559 (0.8); 4.9328 (0.8); 4.9257 (15.5); 4.2804 (2.2); 4.2626 (6.3); 4.2581 (0.6); 4.2447 (6.2); 4.2402
(0.6); 4.2269 (2.0); 1.9345 (7.5); 1.9322 (9.9); 1.9308 (9.7); 1.9285 (7.7); 1.5542 (14.5); 1.3009 (7.8); 1.2954 (1.3); 1.2831 (16.0);
1.2774 (1.3); 1.2653 (7.6); 1.2595 (0.5); 0.0080 (1.3); −0.0002 (52.6); −0.0085 (1.5)
I-266: ¹H-NMR(400.6 MHz, d₆-DMSO):
δ = 13.0994 (1.0); 8.2740 (2.1); 8.2718 (2.6); 8.2708 (2.6); 8.2686 (2.1); 8.2625 (2.2); 8.2602 (2.8); 8.2570 (2.0); 8.2312 (3.3);
8.2249 (3.4); 7.9952 (1.9); 7.9920 (2.7); 7.9863 (1.5); 7.9740 (3.2); 7.9708 (4.5); 7.9667 (3.5); 7.9521 (1.8); 7.9493 (2.2); 7.9457
(3.4); 7.6129 (2.0); 7.6035 (2.3); 7.6013 (2.0); 7.5919 (3.5); 7.5827 (1.7); 7.5804 (1.8); 7.5711 (1.5); 7.2950 (2.1); 7.2893 (2.1);
7.2881 (2.0); 7.2749 (2.0); 7.2736 (2.1); 7.2680 (2.0); 7.2667 (1.9); 4.8684 (16.0); 3.3233 (2.6); 2.5252 (0.8); 2.5205 (1.3); 2.5118
(17.2); 2.5073 (37.7); 2.5026 (52.3); 2.4980 (36.4); 2.4934 (15.7); 1.3563 (0.9); 1.2468 (0.6); 0.8579 (1.1); 0.0080 (2.1); 0.0056
(0.8); 0.0048 (1.0); −0.0002 (65.8); −0.0085 (1.7)
I-267: ¹H-NMR(400.6 MHz, CDCl3):
δ = 8.2135 (0.7); 8.2117 (1.0); 8.2094 (0.8); 8.2075 (0.8); 8.2055 (1.1); 8.2033 (0.8); 8.0399 (0.7); 8.0376 (0.8); 8.0353 (0.8);
8.0331 (0.7); 8.0277 (0.7); 8.0255 (0.8); 8.0231 (0.8); 8.0209 (0.7); 7.8641 (0.6); 7.8579 (0.6); 7.8452 (0.7); 7.8430 (0.8); 7.8390
(0.8); 7.8368 (0.8); 7.8241 (1.0); 7.8209 (0.8); 7.8184 (0.9); 7.8034 (1.2); 7.8010 (1.6); 7.8004 (1.4); 7.7979 (1.0); 7.7839 (1.0);
7.7793 (1.0); 7.7662 (1.0); 7.7615 (1.0); 7.7455 (0.5); 7.7407 (0.6); 7.2620 (8.3); 7.1010 (0.8); 7.0976 (0.9); 7.0888 (0.8); 7.0856
(0.8); 7.0834 (0.8); 7.0800 (0.8); 7.0711 (0.7); 7.0679 (0.7); 6.9944 (0.9); 6.9928 (0.9); 6.9871 (0.9); 6.9855 (0.9); 6.9733 (0.9);
6.9717 (0.9); 6.9659 (0.9); 6.9643 (0.8); 5.3531 (1.7); 5.3356 (1.8); 5.3179 (0.5); 5.3001 (3.6); 5.2121 (1.4); 5.1945 (1.4); 3.7264
(4.0); 3.7239 (16.0); 1.7872 (1.1); 1.7697 (1.2); 1.7635 (5.7); 1.7460 (5.6); 1.5606 (1.4); 1.5429 (1.3); 1.5269 (6.5); 1.5093
(6.4); −0.0002 (12.7)
I-268: ¹H-NMR(400.6 MHz, CDCl3):
δ = 8.1715 (1.8); 8.1694 (1.4); 8.1673 (1.5); 8.1653 (1.9); 8.0537 (1.1); 8.0516 (1.3); 8.0490 (1.3); 8.0468 (1.2); 8.0415 (1.2);
8.0395 (1.3); 8.0368 (1.3); 8.0347 (1.2); 7.8170 (1.0); 7.8108 (1.0); 7.7980 (1.2); 7.7960 (1.2); 7.7918 (1.2); 7.7898 (1.2); 7.7770
(1.1); 7.7708 (1.0); 7.7509 (0.8); 7.7462 (0.8); 7.7325 (1.0); 7.7302 (1.3); 7.7278 (1.1); 7.7255 (1.3); 7.7119 (1.3); 7.7072 (1.3);
7.6371 (1.6); 7.6347 (2.7); 7.6323 (1.6); 7.6164 (1.1); 7.6141 (1.9); 7.6116 (1.0); 7.2614 (21.2); 7.0838 (1.3); 7.0812 (1.3); 7.0717
(1.2); 7.0691 (1.3); 7.0656 (1.3); 7.0629 (1.2); 7.0534 (1.2); 7.0508 (1.2); 6.9889 (1.3); 6.9875 (1.3); 6.9816 (1.3); 6.9802 (1.3);
6.9679 (1.2); 6.9664 (1.3); 6.9606 (1.2); 6.9590 (1.2); 5.3000 (0.5); 4.9240 (3.4); 4.9208 (3.6); 4.9170 (5.1); 4.4180 (0.6); 4.4129
(0.6); 4.4002 (0.6); 4.3951 (0.7); 4.3909 (1.2); 4.3858 (1.2); 4.3731 (1.2); 4.3680 (1.2); 4.3301 (1.2); 4.3271 (1.2); 4.3160 (1.3);
4.3131 (1.2); 4.3030 (0.7); 4.3000 (0.7); 4.2889 (0.7); 4.2860 (0.7); 3.8162 (1.0); 3.7261 (1.4); 3.6942 (0.5); 3.6827 (16.0); 3.6812
(15.8); 2.8597 (0.7); 2.8456 (0.8); 2.8418 (1.2); 2.8277 (1.2); 2.8240 (0.8); 2.8098 (0.8); 2.3563 (0.6); 2.3365 (0.8); 2.3269 (1.4);
2.3071 (1.5); 2.2975 (0.7); 2.2777 (0.7); 1.8341 (0.6); 1.8318 (0.6); 1.8142 (0.6); 1.8120 (0.6); 1.6960 (0.6); 1.6832 (0.6); 1.5582
(1.5); 1.2027 (8.0); 1.2022 (7.9); 1.1981 (1.3); 1.1848 (7.9); 1.1844 (7.7); 1.1799 (1.2); 0.0079 (1.0); −0.0002 (34.1); −0.0028
(1.2); −0.0085 (0.9)
I-269: ¹H-NMR(400.6 MHz, CDCl3):
δ = 8.4890 (0.8); 8.4853 (1.0); 8.4777 (0.9); 8.4733 (1.0); 8.1496 (1.0); 8.1453 (1.0); 8.1296 (1.2); 8.1254 (1.2); 8.1177 (1.6);
8.1157 (1.2); 8.1135 (1.2); 8.1115 (1.6); 7.8548 (0.8); 7.8486 (0.7); 7.8361 (0.9); 7.8335 (1.0); 7.8299 (1.0); 7.8273 (0.8); 7.8149
(0.8); 7.8086 (0.7); 7.4638 (0.8); 7.4621 (0.8); 7.4518 (0.8); 7.4501 (0.8); 7.4440 (0.8); 7.4424 (0.8); 7.4320 (0.7); 7.4304 (0.7);
7.2621 (13.9); 6.9569 (1.0); 6.9554 (1.0); 6.9495 (1.1); 6.9480 (1.1); 6.9356 (1.0); 6.9341 (1.0); 6.9282 (1.0); 6.9267 (1.0); 5.2242
(2.1); 5.2067 (2.1); 5.1893 (0.5); 4.1500 (1.1); 4.1321 (3.2); 4.1143 (3.3); 4.0965 (1.1); 3.7264 (1.4); 2.1063 (6.8); 2.0467 (16.0);
1.7417 (7.3); 1.7361 (0.8); 1.7242 (7.2); 1.7186 (0.7); 1.4321 (1.4); 1.2773 (4.8); 1.2594 (9.8); 1.2416 (4.8); 0.0080 (0.6); −0.0002
(20.4); −0.0085 (0.5)
I-270: ¹H-NMR(400.6 MHz, CDCl3):
δ = 8.2229 (1.4); 8.2209 (1.6); 8.2192 (1.6); 8.2174 (1.4); 8.2112 (1.5); 8.2093 (1.7); 8.2075 (1.6); 8.2057 (1.4); 8.1360 (2.1);
8.1340 (1.6); 8.1319 (1.7); 8.1299 (2.2); 7.7715 (1.1); 7.7653 (1.1); 7.7525 (1.3); 7.7504 (1.4); 7.7463 (1.3); 7.7442 (1.3); 7.7314
(1.2); 7.7252 (1.1); 7.4651 (1.2); 7.4614 (1.2); 7.4445 (1.6); 7.4411 (2.0); 7.4379 (1.3); 7.4210 (1.5); 7.4173 (1.4); 7.2905 (1.5);
7.2815 (1.6); 7.2788 (1.5); 7.2699 (2.7); 7.2614 (28.2); 7.2583 (1.6); 7.2493 (1.1); 6.9215 (1.5); 6.9200 (1.5); 6.9140 (1.5); 6.9125
(1.5); 6.9004 (1.4); 6.8988 (1.4); 6.8929 (1.5); 6.8914 (1.4); 4.9405 (16.0); 4.4301 (2.0); 4.4189 (2.6); 4.4067 (2.4); 4.1743 (4.6);
4.1686 (1.2); 4.1657 (2.1); 4.1623 (4.0); 4.1598 (2.2); 4.1569 (1.2); 4.1507 (3.7); 3.7845 (0.8); 1.6112 (0.5); 1.5977 (0.6); 1.5950
(0.9); 1.5928 (0.9); 1.5909 (0.9); 1.5830 (1.4); 1.5769 (4.0); 1.5639 (1.1); 1.5608 (1.2); 1.5587 (1.0); 1.5567 (0.9); 1.5429 (0.6);
0.8341 (0.5); 0.7864 (1.2); 0.7821 (3.9); 0.7793 (3.4); 0.7767 (3.4); 0.7741 (4.0); 0.7693 (1.4); 0.7609 (16.0); 0.0079 (1.0); −0.0002
(35.4); −0.0085 (0.9)
I-272: ¹H-NMR(400.6 MHz, CDCl3):
δ = 8.1154 (0.5); 7.2632 (5.8); 4.8819 (3.6); 4.4830 (0.9); 4.4668 (1.9); 4.4507 (0.9); 3.6923 (7.0); 3.4905 (16.0); 2.6940 (0.9);
2.6779 (1.8); 2.6618 (0.8); −0.0002 (8.8)
I-273: ¹H-NMR(400.6 MHz, CDCl3):
δ = 8.1536 (1.9); 8.1516 (1.5); 8.1496 (1.5); 8.1475 (1.9); 8.1455 (1.3); 8.0303 (1.1); 8.0282 (1.2); 8.0256 (1.4); 8.0235 (1.2);
8.0182 (1.2); 8.0161 (1.3); 8.0135 (1.3); 8.0114 (1.2); 7.7907 (1.1); 7.7845 (1.1); 7.7716 (1.2); 7.7697 (1.4); 7.7654 (1.2); 7.7635
(1.3); 7.7506 (1.2); 7.7444 (1.1); 7.7298 (1.0); 7.7251 (1.0); 7.7117 (1.1); 7.7091 (1.6); 7.7070 (1.1); 7.7044 (1.6); 7.6910 (1.8);
7.6863 (1.7); 7.6472 (1.6); 7.6447 (2.8); 7.6423 (1.8); 7.6265 (1.0); 7.6241 (1.6); 7.6216 (0.9); 7.2618 (10.8); 7.0561 (1.3); 7.0532
(1.3); 7.0439 (1.3); 7.0411 (1.3); 7.0380 (1.4); 7.0351 (1.2); 7.0258 (1.3); 7.0230 (1.2); 6.9818 (1.3); 6.9803 (1.4); 6.9745 (1.4);
6.9729 (1.4); 6.9608 (1.3); 6.9592 (1.4); 6.9534 (1.3); 6.9519 (1.3); 6.2758 (1.7); 6.2469 (2.1); 6.2315 (2.6); 6.2026 (2.4); 5.9616
(2.6); 5.9569 (2.7); 5.9173 (2.0); 5.9127 (1.9); 5.2668 (2.5); 5.2622 (2.2); 5.2380 (2.1); 5.2333 (2.3); 4.9764 (14.7); 4.3304 (1.8);
4.3126 (5.8); 4.2947 (5.9); 4.2769 (1.9); 1.5727 (2.3); 1.3412 (7.7); 1.3234 (16.0); 1.3056 (7.5); 1.2643 (0.6); 0.8817 (1.3); −0.0002 (15.2)
I-274: ¹H-NMR(400.6 MHz, CDCl3):
δ = 8.2190 (1.8); 8.2081 (1.9); 8.1306 (2.5); 8.1245 (2.6); 7.7708 (1.1); 7.7646 (1.1); 7.7498 (1.5); 7.7453 (1.4); 7.7307 (1.2);
7.7245 (1.1); 7.4619 (1.1); 7.4582 (1.1); 7.4412 (1.5); 7.4380 (2.2); 7.4349 (1.2); 7.4179 (1.4); 7.4142 (1.4); 7.2850 (1.4); 7.2759
(1.7); 7.2734 (1.6); 7.2615 (21.9); 7.2555 (1.5); 7.2529 (1.3); 7.2439 (1.0); 6.9187 (1.7); 6.9116 (1.7); 6.8977 (1.6); 6.8905 (1.6);
4.9345 (16.0); 4.2554 (0.5); 4.2488 (1.6); 4.2308 (2.4); 4.2242 (1.3); 4.2150 (0.5); 4.2109 (0.5); 4.1745 (1.9); 4.1598 (3.6); 4.1463
(1.5); 4.1418 (0.9); 4.1358 (3.4); 4.1307 (1.7); 4.1220 (0.8); 4.1151 (0.7); 3.8902 (0.8); 3.8732 (1.6); 3.8693 (1.4); 3.8566 (1.0);
3.8524 (2.2); 3.8361 (1.1); 3.7959 (1.1); 3.7785 (1.6); 3.7624 (1.3); 3.7578 (1.1); 3.7420 (0.6); 1.9670 (0.7); 1.9502 (0.9); 1.9377
(0.7); 1.9229 (0.6); 1.9159 (0.7); 1.9045 (0.7); 1.9008 (1.0); 1.8963 (0.8); 1.8865 (1.7); 1.8697 (1.6); 1.8659 (1.6); 1.8491 (1.2);
1.6426 (0.8); 1.6295 (0.6); 1.6211 (0.8); 1.6088 (1.0); 1.6046 (0.6); 1.5933 (1.4); 1.5887 (1.1); 1.5816 (0.8); 1.5746 (2.5); 1.5630
(5.4); 1.5553 (1.3); 1.5404 (0.6); 0.7845 (4.7); 0.7797 (5.6); 0.7728 (4.2); 0.7692 (2.5); 0.7629 (2.6); 0.7594 (4.0); 0.0077
(0.9); −0.0002 (31.0); −0.0085 (1.1)
I-275: ¹H-NMR(400.6 MHz, CDCl3):
δ = 8.2024 (0.8); 8.2004 (1.1); 8.1984 (0.8); 8.1963 (0.8); 8.1942 (1.1); 8.1922 (0.7); 8.0656 (0.7); 8.0636 (0.7); 8.0609 (0.8);
8.0588 (0.7); 8.0535 (0.7); 8.0514 (0.8); 8.0488 (0.8); 8.0467 (0.7); 7.8599 (0.6); 7.8537 (0.6); 7.8409 (0.7); 7.8388 (0.9); 7.8348
(0.7); 7.8326 (0.7); 7.8198 (0.7); 7.8136 (0.6); 7.7610 (0.7); 7.7561 (0.6); 7.7425 (0.7); 7.7402 (0.9); 7.7378 (0.7); 7.7355 (0.8);
7.7218 (0.9); 7.7171 (0.8); 7.6274 (0.9); 7.6250 (1.6); 7.6227 (1.0); 7.6067 (0.7); 7.6044 (1.2); 7.6020 (0.7); 7.2620 (8.6); 7.1092
(0.8); 7.1066 (0.8); 7.0971 (0.8); 7.0944 (0.8); 7.0908 (0.8); 7.0882 (0.8); 7.0787 (0.8); 7.0760 (0.7); 6.9942 (0.9); 6.9927 (0.9);
6.9869 (0.9); 6.9854 (0.9); 6.9731 (0.9); 6.9716 (0.9); 6.9657 (0.9); 6.9642 (0.8); 5.3341 (1.8); 5.3166 (1.8); 5.3000 (3.8); 5.2536
(1.4); 5.2360 (1.5); 3.8014 (0.5); 3.7264 (16.0); 3.7239 (4.1); 1.7871 (5.8); 1.7697 (5.7); 1.7634 (1.4); 1.7459 (1.2); 1.5605 (6.7);
1.5428 (6.6); 1.5268 (1.4); 1.5092 (1.3); −0.0002 (12.1)
I-276: ¹H-NMR(400.6 MHz, CDCl3):
δ = 8.2272 (1.2); 8.2253 (1.0); 8.2232 (1.0); 8.2210 (1.2); 7.8837 (0.6); 7.8775 (0.6); 7.8647 (0.7); 7.8626 (0.8); 7.8585 (0.7);
7.8565 (0.8); 7.8436 (0.7); 7.8374 (0.6); 7.6472 (1.0); 7.6274 (1.6); 7.6081 (1.3); 7.4343 (1.4); 7.4141 (1.2); 7.2610 (19.6); 6.9965
(0.8); 6.9950 (0.9); 6.9891 (0.8); 6.9877 (0.9); 6.9753 (0.8); 6.9738 (0.9); 6.9679 (0.8); 6.9665 (0.8); 6.9523 (1.3); 6.9335 (1.2);
4.9450 (8.5); 4.5216 (2.0); 4.5058 (4.2); 4.4901 (2.1); 3.6867 (16.0); 2.7183 (2.0); 2.7026 (4.0); 2.6868 (1.9); 2.1562 (9.5); 1.5699
(1.8); 0.0080 (0.8); −0.0002 (28.0); −0.0085 (0.7)
I-277: ¹H-NMR(400.6 MHz, CDCl3):
δ = 8.2103 (0.9); 8.2081 (0.7); 8.2060 (0.8); 8.2043 (0.9); 7.8703 (0.5); 7.8513 (0.6); 7.8492 (0.6); 7.8451 (0.6); 7.8430 (0.6);
7.8302 (0.5); 7.8240 (0.5); 7.6302 (0.8); 7.6104 (1.2); 7.5910 (1.0); 7.4357 (1.1); 7.4154 (0.8); 7.2613 (10.4); 6.9944 (0.6); 6.9929
(0.6); 6.9870 (0.7); 6.9856 (0.6); 6.9733 (0.6); 6.9717 (0.6); 6.9659 (0.6); 6.9644 (0.6); 6.9403 (1.0); 6.9216 (1.0); 4.9978 (6.7);
4.3947 (1.6); 4.3865 (0.8); 4.3831 (1.6); 4.3793 (0.8); 4.3713 (1.7); 3.6503 (2.0); 3.6436 (0.7); 3.6423 (0.9); 3.6386 (1.7); 3.6351
(0.9); 3.6270 (1.8); 3.3802 (16.0); 2.1428 (7.3); 1.5668 (1.2); −0.0002 (13.9); −0.0027 (0.6)
I-278: ¹H-NMR(400.6 MHz, CDCl3):
δ = 8.2207 (0.7); 8.2188 (1.1); 8.2168 (0.9); 8.2147 (0.9); 8.2126 (1.1); 8.2107 (0.8); 7.8773 (0.6); 7.8712 (0.6); 7.8583 (0.7);
7.8562 (0.8); 7.8522 (0.7); 7.8501 (0.8); 7.8372 (0.7); 7.8311 (0.7); 7.6438 (1.0); 7.6239 (1.5); 7.6047 (1.2); 7.4378 (1.3); 7.4175
(1.1); 7.2617 (11.6); 6.9955 (0.8); 6.9940 (0.8); 6.9881 (0.8); 6.9866 (0.8); 6.9744 (0.8); 6.9729 (0.8); 6.9670 (0.8); 6.9655 (0.8);
6.9471 (1.2); 6.9284 (1.1); 4.9437 (8.1); 4.5198 (2.0); 4.5040 (4.1); 4.4883 (2.0); 3.6867 (16.0); 2.7171 (1.9); 2.7014 (3.9); 2.6856
(1.8); 2.1463 (8.9); 1.5779 (1.0); −0.0002 (16.7)
I-279: ¹H-NMR(400.6 MHz, CDCl3):
δ = 8.2196 (0.9); 8.2176 (0.7); 8.2156 (0.7); 8.2135 (0.9); 7.8776 (0.5); 7.8586 (0.6); 7.8565 (0.6); 7.8524 (0.6); 7.8503 (0.6);
7.8375 (0.6); 7.8313 (0.5); 7.6341 (0.8); 7.6142 (1.2); 7.5950 (1.0); 7.4318 (1.0); 7.4115 (0.8); 7.2605 (37.0); 7.2564 (1.0); 7.2547
(0.6); 6.9956 (0.7); 6.9940 (0.7); 6.9881 (0.7); 6.9866 (0.7); 6.9744 (0.6); 6.9729 (0.6); 6.9670 (0.6); 6.9655 (0.6); 6.9457 (1.0);
6.9270 (0.9); 4.9989 (6.7); 4.3965 (1.6); 4.3885 (0.8); 4.3850 (1.6); 4.3812 (0.8); 4.3732 (1.7); 3.6508 (2.0); 3.6428 (0.9); 3.6390
(1.7); 3.6356 (0.9); 3.6274 (1.9); 3.3807 (16.0); 2.1530 (7.2); 1.5634 (3.4); 0.0080 (1.4); −0.0002 (53.3); −0.0085 (1.5)
I-280: ¹H-NMR(400.6 MHz, CDCl3):
δ = 8.5199 (1.8); 8.5066 (1.8); 8.2588 (1.4); 8.2569 (1.7); 8.2552 (1.7); 8.2534 (1.5); 8.2472 (1.5); 8.2453 (1.7); 8.2435 (1.7);
8.2418 (1.5); 8.1399 (2.4); 8.1338 (2.5); 7.7601 (1.2); 7.7538 (1.2); 7.7411 (1.4); 7.7390 (1.5); 7.7349 (1.4); 7.7328 (1.5); 7.7201
(1.3); 7.7139 (1.2); 7.5198 (1.0); 7.4808 (1.2); 7.4771 (1.2); 7.4602 (1.7); 7.4569 (2.1); 7.4538 (1.4); 7.4369 (1.6); 7.4331 (1.6);
7.3269 (1.5); 7.3179 (1.7); 7.3154 (1.6); 7.3063 (2.8); 7.2971 (3.2); 7.2947 (3.8); 7.2856 (3.0); 7.2813 (2.5); 7.2741 (0.8); 7.2734
(0.8); 7.2709 (0.6); 7.2701 (0.7); 7.2693 (0.8); 7.2686 (0.9); 7.2677 (0.8); 7.2614 (192.2); 7.2564 (3.1); 7.2540 (1.0); 7.2523 (0.6);
7.2514 (0.5); 7.2507 (0.7); 6.9977 (1.0); 6.9267 (1.5); 6.9253 (1.7); 6.9193 (1.6); 6.9178 (1.6); 6.9055 (1.6); 6.9041 (1.7); 6.8981
(1.6); 6.8966 (1.6); 5.2688 (8.8); 5.0016 (16.0); 4.9501 (1.1); 1.6175 (0.6); 1.6032 (0.9); 1.5969 (1.1); 1.5910 (0.8); 1.5832 (2.0);
1.5769 (0.8); 1.5690 (0.9); 1.5628 (1.1); 1.5491 (0.6); 0.8042 (0.6); 0.7887 (1.7); 0.7843 (2.7); 0.7779 (2.7); 0.7681 (1.8); 0.7654
(2.1); 0.7625 (5.7); 0.7578 (4.4); 0.7515 (2.0); 0.7484 (2.6); 0.7441 (3.6); 0.7410 (1.9); 0.7383 (1.9); 0.0080 (3.2); −0.0002
(115.2); −0.0085 (3.0)
I-281: ¹H-NMR(400.6 MHz, CDCl3):
δ = 8.4749 (0.7); 8.4713 (0.7); 8.4629 (0.8); 8.4592 (0.8); 8.1488 (0.8); 8.1449 (0.8); 8.1290 (0.9); 8.1251 (0.9); 8.1140 (1.1);
8.1120 (0.9); 8.1099 (0.9); 8.1077 (1.2); 8.1059 (0.8); 7.8486 (0.6); 7.8424 (0.6); 7.8299 (0.7); 7.8274 (0.8); 7.8237 (0.7); 7.8211
(0.8); 7.8087 (0.7); 7.8024 (0.7); 7.4528 (0.6); 7.4509 (0.6); 7.4408 (0.6); 7.4389 (0.6); 7.4329 (0.6); 7.4312 (0.6); 7.4210 (0.6);
7.4192 (0.6); 7.2623 (10.2); 6.9518 (0.8); 6.9502 (0.9); 6.9443 (0.9); 6.9428 (0.9); 6.9306 (0.8); 6.9290 (0.8); 6.9231 (0.8); 6.9215
(0.8); 5.3004 (5.0); 5.1942 (1.6); 5.1768 (1.6); 3.7562 (16.0); 3.7496 (0.9); 1.6903 (6.7); 1.6840 (0.6); 1.6729 (6.6); 1.6666 (0.5);
1.5657 (7.6); −0.0002 (11.5)
I-282: ¹H-NMR(400.6 MHz, CDCl3):
δ = 8.5791 (1.2); 8.5768 (1.3); 8.5748 (1.4); 8.5726 (1.2); 8.5669 (1.3); 8.5646 (1.4); 8.5626 (1.4); 8.5604 (1.2); 8.2347 (1.4);
8.2328 (1.7); 8.2311 (1.7); 8.2292 (1.5); 8.2231 (1.5); 8.2212 (1.8); 8.2195 (1.7); 8.2177 (1.5); 8.1307 (2.2); 8.1286 (1.7); 8.1264
(1.8); 8.1245 (2.4); 7.7718 (1.1); 7.7656 (1.1); 7.7528 (1.3); 7.7507 (1.4); 7.7466 (1.4); 7.7445 (1.4); 7.7317 (1.2); 7.7255 (1.2);
7.6501 (0.7); 7.6457 (0.7); 7.6307 (1.5); 7.6264 (1.5); 7.6115 (0.9); 7.6071 (0.9); 7.4675 (1.2); 7.4638 (1.2); 7.4469 (1.6); 7.4436
(2.0); 7.4404 (1.4); 7.4235 (1.6); 7.4198 (1.6); 7.3711 (1.7); 7.3516 (1.6); 7.3000 (1.4); 7.2911 (1.8); 7.2884 (1.5); 7.2795 (2.6);
7.2704 (1.3); 7.2678 (1.6); 7.2661 (1.2); 7.2621 (71.9); 7.2596 (3.6); 7.2571 (1.2); 7.2563 (0.9); 7.2555 (0.8); 7.2547 (0.6); 7.2539
(0.5); 7.2443 (1.0); 7.2435 (1.0); 7.2428 (1.0); 7.2293 (1.0); 7.2239 (1.0); 7.2126 (0.8); 6.9238 (1.5); 6.9223 (1.6); 6.9164 (1.6);
6.9148 (1.5); 6.9027 (1.5); 6.9012 (1.5); 6.8952 (1.5); 6.8937 (1.5); 5.3675 (9.2); 5.0180 (15.8); 1.5934 (1.6); 1.5764 (2.3); 1.5657
(0.6); 1.5593 (1.8); 1.5423 (0.6); 0.7728 (16.0); 0.7610 (1.1); 0.7557 (14.3); 0.0080 (1.2); 0.0040 (0.5); −0.0002 (46.5); −0.0049
(0.7); −0.0057 (0.6); −0.0066 (0.6); −0.0085 (1.3)
I-283: ¹H-NMR(400.6 MHz, CDCl3):
δ = 8.4706 (0.9); 8.4587 (0.9); 8.1471 (0.9); 8.1429 (0.8); 8.1273 (1.0); 8.1231 (1.0); 8.1100 (1.3); 8.1048 (1.3); 7.8488 (0.6);
7.8426 (0.6); 7.8300 (0.8); 7.8276 (0.8); 7.8238 (0.8); 7.8214 (0.8); 7.8089 (0.7); 7.8026 (0.7); 7.4494 (0.7); 7.4374 (0.7); 7.4296
(0.7); 7.4177 (0.6); 7.2618 (16.2); 6.9525 (1.0); 6.9512 (0.9); 6.9450 (1.0); 6.9312 (1.0); 6.9300 (0.9); 6.9238 (1.0); 5.1847 (1.2);
5.1835 (1.1); 5.1672 (1.2); 5.1660 (1.1); 4.3780 (0.6); 4.3601 (0.6); 4.3509 (0.8); 4.3330 (0.8); 4.2868 (1.2); 4.2792 (1.1); 4.2699
(1.2); 4.2636 (1.2); 4.2152 (0.8); 4.2003 (0.8); 4.1881 (0.6); 4.1732 (0.6); 3.7563 (0.8); 3.7265 (2.0); 3.7136 (0.8); 3.6741 (1.0);
3.6689 (16.0); 2.7978 (0.9); 2.7803 (0.8); 1.6741 (5.7); 1.6568 (5.6); 1.5584 (5.0); 1.1982 (1.0); 1.1800 (1.0); 1.1618 (4.2); 1.1576
(4.4); 1.1439 (4.2); 1.1398 (4.1); 0.0080 (0.7); −0.0002 (24.1); −0.0085 (0.6)
I-287: ¹H-NMR(400.6 MHz, CDCl3):
δ = 8.2283 (1.7); 8.2167 (1.7); 8.1304 (2.3); 8.1242 (2.3); 7.7679 (1.0); 7.7617 (1.0); 7.7469 (1.3); 7.7425 (1.3); 7.7278 (1.1);
7.7216 (1.0); 7.4623 (1.1); 7.4586 (1.0); 7.4417 (1.5); 7.4385 (2.0); 7.4352 (1.1); 7.4183 (1.4); 7.4146 (1.3); 7.3381 (1.5); 7.3302
(3.3); 7.3244 (8.6); 7.3205 (8.4); 7.3153 (2.8); 7.3105 (2.7); 7.3068 (2.0); 7.3005 (1.2); 7.2905 (1.4); 7.2815 (1.5); 7.2788 (1.4);
7.2699 (2.4); 7.2601 (25.4); 7.2493 (1.0); 6.9202 (1.6); 6.9127 (1.6); 6.8990 (1.5); 6.8916 (1.5); 5.2228 (13.8); 4.9489 (15.0);
1.5797 (1.3); 1.5630 (2.0); 1.5496 (2.6); 1.5287 (0.6); 0.7578 (10.8); 0.7407 (16.0); 0.0079 (1.1); −0.0002 (34.6); −0.0085 (1.0)
I-289: ¹H-NMR(400.6 MHz, CDCl3):
δ = 8.2292 (1.6); 8.2273 (1.8); 8.2257 (1.8); 8.2175 (1.7); 8.2157 (1.9); 8.1354 (2.5); 8.1293 (2.5); 7.7712 (1.1); 7.7650 (1.1);
7.7521 (1.5); 7.7502 (1.4); 7.7459 (1.4); 7.7441 (1.3); 7.7312 (1.2); 7.7250 (1.2); 7.4666 (1.2); 7.4629 (1.2); 7.4459 (1.7); 7.4427
(2.2); 7.4395 (1.2); 7.4226 (1.5); 7.4188 (1.4); 7.2918 (1.4); 7.2828 (1.7); 7.2802 (1.5); 7.2712 (2.7); 7.2612 (31.0); 7.2506 (1.2);
6.9214 (1.7); 6.9140 (1.7); 6.9004 (1.6); 6.8930 (1.6); 4.8961 (16.0); 4.2290 (1.4); 4.2127 (1.5); 4.2021 (2.2); 4.1857 (2.2); 4.1093
(2.3); 4.0894 (2.4); 4.0824 (1.5); 4.0624 (1.5); 3.8483 (0.7); 3.8346 (0.8); 3.8275 (1.6); 3.8137 (1.7); 3.8087 (2.3); 3.7917 (2.2);
3.7869 (2.1); 3.7689 (2.0); 3.7464 (1.2); 3.7286 (1.9); 3.7099 (1.6); 3.6888 (0.8); 3.5447 (2.0); 3.5307 (2.0); 3.5224 (1.7); 3.5085
(1.7); 2.6070 (0.7); 2.5904 (0.8); 2.5730 (0.6); 2.0176 (0.6); 2.0050 (0.9); 1.9965 (0.5); 1.9920 (0.6); 1.9841 (0.8); 1.9720 (0.7);
1.9653 (0.6); 1.6466 (0.5); 1.6300 (0.8); 1.6132 (1.2); 1.5958 (2.5); 1.5897 (0.6); 1.5789 (3.0); 1.5681 (0.8); 1.5617 (2.6); 1.5554
(7.5); 1.5447 (0.8); 0.7839 (15.7); 0.7668 (14.6); 0.0080 (1.3); −0.0002 (42.8); −0.0084 (1.1)
I-291: ¹H-NMR(400.6 MHz, CDCl3):
δ = 8.2143 (2.1); 8.2113 (1.7); 8.2083 (2.2); 8.0584 (0.8); 8.0563 (0.9); 8.0537 (1.5); 8.0517 (1.5); 8.0490 (1.0); 8.0466 (1.4);
8.0442 (1.0); 8.0415 (1.5); 8.0395 (1.5); 8.0369 (0.9); 8.0348 (0.8); 7.8689 (1.1); 7.8628 (1.1); 7.8499 (1.3); 7.8479 (1.4); 7.8438
(1.3); 7.8418 (1.3); 7.8289 (1.1); 7.8228 (1.1); 7.7804 (0.6); 7.7756 (0.6); 7.7720 (0.6); 7.7673 (0.6); 7.7619 (0.7); 7.7597 (0.9);
7.7573 (0.8); 7.7547 (1.0); 7.7513 (1.0); 7.7489 (0.8); 7.7466 (0.9); 7.7413 (0.9); 7.7365 (0.9); 7.7330 (0.9); 7.7282 (0.8); 7.6511
(0.9); 7.6489 (1.7); 7.6461 (1.5); 7.6434 (1.7); 7.6411 (1.0); 7.6305 (0.7); 7.6282 (1.2); 7.6254 (1.2); 7.6228 (1.2); 7.6204 (0.7);
7.2614 (19.9); 7.1055 (1.2); 7.0933 (1.3); 7.0917 (1.1); 7.0894 (1.0); 7.0877 (1.2); 7.0756 (1.1); 6.9937 (1.7); 6.9876 (1.6); 6.9863
(1.6); 6.9738 (1.5); 6.9725 (1.6); 6.9665 (1.5); 6.9651 (1.5); 5.2612 (1.6); 5.2529 (1.6); 5.2438 (1.6); 5.2355 (1.6); 4.4105 (0.9);
4.3925 (0.9); 4.3834 (1.3); 4.3654 (1.4); 4.3363 (2.1); 4.3326 (2.1); 4.3192 (2.5); 4.3176 (2.5); 4.2805 (1.3); 4.2664 (1.4); 4.2534
(0.9); 4.2393 (0.9); 3.8013 (1.0); 3.6555 (16.0); 3.6484 (16.0); 2.8406 (0.5); 2.8377 (0.6); 2.8339 (0.8); 2.8232 (0.8); 2.8198 (0.8);
2.8160 (0.6); 2.8076 (0.8); 2.8019 (0.6); 1.7210 (0.5); 1.7077 (6.7); 1.7056 (6.8); 1.6902 (6.6); 1.6881 (6.7); 1.5567 (2.2); 1.1889
(6.6); 1.1709 (12.8); 1.1529 (6.4); 0.0080 (0.9); −0.0002 (30.3); −0.0085 (0.8)
I-293: ¹H-NMR(400.6 MHz, CDCl3):
δ = 8.1700 (1.2); 8.1639 (1.2); 8.0432 (0.8); 8.0413 (0.8); 8.0386 (0.9); 8.0366 (0.8); 8.0311 (0.8); 8.0292 (0.9); 8.0265 (0.9);
8.0245 (0.8); 7.8366 (0.7); 7.8306 (0.6); 7.8172 (0.5); 7.7921 (0.6); 7.7873 (0.6); 7.7735 (0.8); 7.7715 (1.0); 7.7688 (0.8); 7.7668
(0.9); 7.7530 (0.9); 7.7483 (0.9); 7.6524 (1.0); 7.6501 (1.7); 7.6479 (1.0); 7.6318 (0.8); 7.6296 (1.3); 7.6274 (0.7); 7.2609 (15.0);
7.1421 (0.8); 7.1396 (0.8); 7.1300 (0.8); 7.1275 (0.8); 7.1236 (0.8); 7.1211 (0.8); 7.1116 (0.8); 7.1090 (0.7); 6.9806 (0.9); 6.9733
(0.9); 6.9595 (0.8); 6.9522 (0.8); 5.2507 (1.6); 5.2333 (1.7); 4.4858 (0.9); 4.4694 (2.1); 4.4542 (2.2); 4.4380 (0.9); 3.6597 (0.9);
3.6502 (16.0); 2.6794 (1.1); 2.6761 (1.1); 2.6617 (1.9); 2.6486 (1.0); 2.6451 (1.0); 1.6909 (6.3); 1.6845 (0.6); 1.6735 (6.2); 1.5482
(6.1); 0.0079 (0.7); −0.0002 (21.0); −0.0085 (0.6)
I-294: ¹H-NMR(400.6 MHz, CDCl3):
δ = 8.2242 (1.7); 8.2223 (1.8); 8.2206 (1.7); 8.2125 (1.8); 8.2106 (1.8); 8.2090 (1.6); 8.1457 (2.4); 8.1434 (1.9); 8.1413 (2.2);
8.1396 (2.2); 7.7787 (1.0); 7.7725 (0.9); 7.7597 (1.5); 7.7576 (1.2); 7.7535 (1.4); 7.7515 (1.1); 7.7387 (1.0); 7.7325 (0.9); 7.4797
(1.1); 7.4761 (0.9); 7.4590 (1.7); 7.4560 (1.9); 7.4526 (1.0); 7.4356 (1.3); 7.4320 (1.1); 7.3018 (1.2); 7.2927 (1.7); 7.2901 (1.2);
7.2812 (2.0); 7.2721 (1.4); 7.2695 (1.1); 7.2617 (12.4); 6.9261 (1.6); 6.9186 (1.6); 6.9050 (1.6); 6.8975 (1.5); 4.9825 (13.7);
4.8234 (16.0); 3.7849 (0.7); 2.0450 (0.6); 1.5997 (0.8); 1.5922 (0.9); 1.5865 (0.6); 1.5788 (1.6); 1.5757 (0.9); 1.5734 (0.8); 1.5647
(2.1); 1.5446 (0.5); 1.2770 (0.5); 1.2653 (1.0); 1.2594 (0.9); 0.8991 (0.5); 0.8820 (1.4); 0.8644 (0.6); 0.8112 (0.6); 0.7964 (1.8);
0.7933 (2.5); 0.7859 (1.9); 0.7760 (1.8); 0.7729 (2.1); 0.7707 (2.3); 0.7672 (1.7); 0.7643 (1.9); 0.7602 (2.3); 0.7537 (2.4); 0.7481
(2.5); 0.7467 (2.6); 0.7431 (1.9); 0.7407 (2.3); 0.7377 (1.8); 0.7351 (1.8); 0.0080 (0.7); −0.0002 (18.5)
I-295: ¹H-NMR(400.6 MHz, CDCl3):
δ = 8.2650 (1.6); 8.2632 (1.9); 8.2613 (1.9); 8.2596 (1.7); 8.2533 (1.7); 8.2515 (1.9); 8.2496 (1.9); 8.2480 (1.7); 8.0775 (2.4);
8.0714 (2.5); 7.7524 (1.1); 7.7462 (1.0); 7.7334 (1.3); 7.7313 (1.4); 7.7272 (1.3); 7.7252 (1.3); 7.7124 (1.2); 7.7062 (1.2); 7.4729
(1.3); 7.4692 (1.3); 7.4522 (1.8); 7.4498 (1.8); 7.4486 (2.0); 7.4463 (1.5); 7.4293 (1.7); 7.4255 (1.6); 7.3226 (1.7); 7.3135 (1.9);
7.3110 (1.8); 7.3019 (2.9); 7.2928 (1.3); 7.2903 (1.4); 7.2812 (1.2); 7.2622 (17.5); 6.8943 (1.6); 6.8929 (1.7); 6.8871 (1.7); 6.8856
(1.6); 6.8733 (1.6); 6.8718 (1.7); 6.8659 (1.6); 6.8644 (1.6); 5.3001 (1.2); 5.1370 (0.7); 5.1331 (2.4); 5.1289 (3.5); 5.1248 (2.6);
5.1210 (0.8); 5.0591 (2.6); 5.0568 (3.3); 5.0545 (3.2); 5.0522 (2.5); 5.0135 (0.6); 4.9866 (0.7); 4.9786 (16.0); 1.9126 (8.5); 1.9103
(11.5); 1.9090 (11.7); 1.9067 (9.2); 1.5007 (0.6); 1.4321 (1.6); 1.2865 (0.5); 1.2645 (2.0); 0.8987 (1.1); 0.8818 (4.2); 0.8641 (1.5);
0.0080 (0.6); −0.0002 (22.9); −0.0085 (0.6)
I-296: ¹H-NMR(400.6 MHz, CDCl3):
δ = 8.1542 (3.5); 8.1480 (3.5); 7.8252 (1.6); 7.8190 (1.6); 7.8063 (2.0); 7.8042 (2.1); 7.8001 (1.9); 7.7980 (1.8); 7.7853 (1.7);
7.7791 (1.6); 7.5685 (1.5); 7.5534 (1.6); 7.5475 (2.3); 7.5327 (2.3); 7.5263 (1.6); 7.5113 (1.5); 7.2607 (38.9); 6.9712 (2.3); 6.9699
(2.2); 6.9638 (2.4); 6.9500 (2.2); 6.9488 (2.2); 6.9427 (2.2); 6.8644 (1.7); 6.8583 (1.9); 6.8551 (1.8); 6.8489 (1.7); 6.8427 (1.6);
6.8365 (1.6); 6.8334 (1.6); 6.8272 (1.4); 5.3051 (1.2); 5.2877 (4.9); 5.2703 (4.9); 5.2530 (1.2); 1.6879 (16.0); 1.6705 (15.9);
1.5637 (0.7); 1.5485 (1.1); 1.5449 (1.1); 1.5347 (0.8); 1.5299 (2.7); 1.5227 (0.8); 1.5162 (0.9); 1.5121 (1.5); 1.5086 (0.9); 1.4952
(0.8); 1.4322 (3.2); 1.2643 (1.7); 1.2424 (0.7); 0.8988 (0.9); 0.8820 (3.1); 0.8643 (1.2); 0.7962 (0.7); 0.7854 (0.9); 0.7829 (1.1);
0.7775 (4.5); 0.7672 (10.6); 0.7609 (3.2); 0.7569 (3.0); 0.7527 (4.5); 0.7471 (12.0); 0.7426 (2.2); 0.7258 (0.6); 0.0079
(1.7); −0.0002 (56.6); −0.0085 (1.5)
I-298: ¹H-NMR(400.6 MHz, CDCl3):
δ = 8.1482 (1.2); 8.1420 (1.2); 7.8182 (0.6); 7.8120 (0.6); 7.7992 (0.7); 7.7972 (0.7); 7.7930 (0.7); 7.7910 (0.7); 7.7782 (0.6);
7.7720 (0.6); 7.5606 (0.6); 7.5455 (0.6); 7.5393 (0.9); 7.5243 (0.9); 7.5184 (0.6); 7.5033 (0.6); 7.2618 (6.6); 6.9592 (0.8); 6.9530
(0.8); 6.9518 (0.8); 6.9393 (0.8); 6.9381 (0.8); 6.9319 (0.8); 6.9306 (0.8); 6.8395 (0.6); 6.8334 (0.7); 6.8301 (0.6); 6.8240 (0.6);
6.8178 (0.6); 6.8117 (0.6); 6.8083 (0.6); 6.8023 (0.5); 5.2297 (1.6); 5.2124 (1.6); 3.7622 (16.0); 1.6362 (6.5); 1.6188 (6.5); 1.5580
(2.3); 1.5521 (0.6); 1.5444 (0.6); 1.5313 (0.9); 1.5177 (0.5); 1.5101 (0.6); 0.8194 (0.9); 0.8172 (0.9); 0.8126 (1.4); 0.8104 (1.1);
0.8066 (0.7); 0.8037 (0.6); 0.7995 (0.9); 0.7971 (1.2); 0.7748 (0.6); 0.7640 (1.6); 0.7606 (0.9); 0.7582 (0.7); 0.7557 (0.6); 0.7541
(0.6); 0.7436 (1.5); 0.7388 (0.8); −0.0002 (10.2)
I-300: ¹H-NMR(400.6 MHz, CDCl3):
δ = 8.1503 (1.3); 8.1441 (1.2); 7.8201 (0.7); 7.8139 (0.6); 7.8012 (0.8); 7.7991 (0.8); 7.7950 (0.8); 7.7928 (0.8); 7.7801 (0.7);
7.7739 (0.7); 7.5638 (0.6); 7.5487 (0.7); 7.5424 (0.8); 7.5274 (0.8); 7.5217 (0.7); 7.5067 (0.6); 7.2606 (22.6); 6.9649 (0.8); 6.9635
(0.9); 6.9575 (0.9); 6.9560 (0.9); 6.9438 (0.8); 6.9424 (0.8); 6.9364 (0.8); 6.9349 (0.8); 6.8695 (0.6); 6.8634 (0.7); 6.8602 (0.7);
6.8540 (0.7); 6.8478 (0.6); 6.8416 (0.6); 6.8384 (0.6); 6.8323 (0.6); 4.8869 (8.7); 3.7920 (16.0); 1.5491 (1.4); 1.5422 (11.2);
1.5297 (0.5); 0.7895 (0.8); 0.7848 (2.5); 0.7827 (2.4); 0.7804 (2.4); 0.7781 (2.7); 0.7739 (1.6); 0.7712 (2.3); 0.7674 (1.4); 0.7616
(1.5); 0.7579 (2.2); 0.7548 (0.9); 0.7525 (0.9); 0.0080 (1.0); −0.0002 (32.6); −0.0085 (0.9)
I-301: ¹H-NMR(400.6 MHz, CDCl3):
δ = 8.2616 (0.7); 8.2598 (0.8); 8.2581 (0.8); 8.2563 (0.7); 8.2500 (0.7); 8.2482 (0.8); 8.2465 (0.8); 8.2448 (0.7); 8.1459 (1.2);
8.1439 (0.9); 8.1418 (0.9); 8.1397 (1.2); 7.7586 (0.6); 7.7524 (0.6); 7.7397 (0.7); 7.7376 (0.7); 7.7335 (0.7); 7.7313 (0.7); 7.7186
(0.6); 7.7124 (0.6); 7.4876 (0.6); 7.4838 (0.6); 7.4669 (0.8); 7.4634 (1.0); 7.4607 (0.7); 7.4437 (0.8); 7.4400 (0.8); 7.3261 (0.6);
7.3170 (0.7); 7.3144 (0.7); 7.3054 (1.1); 7.2964 (0.5); 7.2938 (0.5); 7.2619 (18.2); 6.9265 (0.8); 6.9250 (0.8); 6.9191 (0.8); 6.9176
(0.8); 6.9054 (0.8); 6.9039 (0.8); 6.8979 (0.8); 6.8964 (0.8); 4.8090 (7.1); 3.7065 (16.0); 3.6744 (0.7); 3.6590 (1.6); 3.6444 (1.7);
3.6290 (0.7); 2.6197 (1.6); 2.6044 (2.1); 2.5899 (1.4); 1.6348 (0.6); 1.6269 (0.5); 1.6138 (1.1); 1.6006 (0.6); 1.5928 (0.6); 1.5656
(6.1); 0.8573 (0.6); 0.8465 (1.4); 0.8415 (1.6); 0.8363 (0.8); 0.8308 (0.8); 0.8251 (1.6); 0.8204 (1.3); 0.8099 (0.8); 0.6231 (0.8);
0.6124 (1.5); 0.6096 (1.2); 0.6076 (1.6); 0.5993 (1.4); 0.5946 (1.6); 0.5835 (0.6); 0.0079 (0.7); −0.0002 (28.2); −0.0085 (0.8)
I-302: ¹H-NMR(400.6 MHz, d₆-DMSO):
δ = 12.9466 (2.6); 8.2322 (3.5); 8.2260 (3.6); 8.1267 (1.2); 8.1111 (1.3); 8.1046 (2.4); 8.0891 (2.4); 8.0826 (1.3); 8.0670 (1.2);
7.9683 (1.4); 7.9620 (1.4); 7.9473 (2.1); 7.9409 (2.0); 7.9276 (1.5); 7.9213 (1.4); 7.3225 (1.3); 7.3161 (1.8); 7.3143 (1.8); 7.3078
(1.4); 7.3004 (1.3); 7.2922 (1.6); 7.2858 (1.2); 7.2615 (2.2); 7.2545 (2.2); 7.2402 (2.2); 7.2332 (2.1); 5.7565 (1.7); 4.7784 (16.0);
3.3185 (4.9); 2.6704 (0.6); 2.5241 (1.5); 2.5194 (2.3); 2.5106 (34.4); 2.5061 (75.3); 2.5015 (105.6); 2.4968 (73.4); 2.4923 (32.4);
2.4734 (0.5); 2.3286 (0.6); 1.6644 (0.6); 1.6513 (1.4); 1.6433 (1.4); 1.6384 (0.9); 1.6303 (2.8); 1.6225 (0.8); 1.6172 (1.5); 1.6093
(1.5); 1.5962 (0.8); 0.7519 (1.4); 0.7406 (3.0); 0.7352 (4.1); 0.7309 (1.9); 0.7263 (2.4); 0.7197 (3.1); 0.7140 (3.9); 0.7054 (2.0);
0.6380 (2.1); 0.6289 (4.0); 0.6240 (4.4); 0.6160 (4.0); 0.6107 (3.5); 0.5994 (1.3); 0.0080 (3.0); −0.0002 (125.7); −0.0085 (3.7)
I-303: ¹H-NMR(400.6 MHz, CDCl3):
δ = 8.2678 (1.2); 8.2659 (1.4); 8.2641 (1.5); 8.2623 (1.3); 8.2562 (1.3); 8.2543 (1.5); 8.2525 (1.5); 8.2508 (1.3); 8.1498 (2.0);
8.1479 (1.5); 8.1456 (1.6); 8.1436 (2.0); 7.7676 (1.0); 7.7614 (1.0); 7.7487 (1.1); 7.7465 (1.2); 7.7424 (1.1); 7.7403 (1.2); 7.7276
(1.0); 7.7214 (1.0); 7.4881 (1.0); 7.4844 (1.1); 7.4674 (1.4); 7.4639 (1.7); 7.4612 (1.2); 7.4443 (1.4); 7.4405 (1.3); 7.3282 (1.3);
7.3191 (1.4); 7.3166 (1.3); 7.3075 (2.2); 7.2984 (1.0); 7.2960 (1.1); 7.2868 (1.0); 7.2627 (60.6); 7.2068 (0.7); 7.1893 (0.7); 6.9292
(1.3); 6.9277 (1.4); 6.9218 (1.4); 6.9203 (1.3); 6.9081 (1.2); 6.9066 (1.3); 6.9006 (1.3); 6.8991 (1.3); 4.8868 (1.4); 4.8485 (5.4);
4.8261 (5.8); 4.7878 (1.5); 4.6871 (1.3); 4.6691 (2.0); 4.6511 (1.4); 4.2673 (2.0); 4.2495 (6.5); 4.2317 (6.7); 4.2138 (2.1); 4.1309
(0.5); 4.1131 (0.6); 2.0454 (2.7); 1.6557 (1.0); 1.6478 (0.9); 1.6426 (0.6); 1.6348 (1.9); 1.6269 (0.6); 1.6216 (1.0); 1.6138 (1.1);
1.6006 (0.7); 1.5819 (2.8); 1.4952 (10.8); 1.4774 (10.8); 1.3209 (7.5); 1.3031 (16.0); 1.2852 (7.5); 1.2774 (1.1); 1.2596 (2.1);
1.2417 (0.9); 0.8819 (1.1); 0.8775 (0.6); 0.8731 (0.6); 0.8663 (2.2); 0.8612 (2.6); 0.8564 (0.8); 0.8545 (0.8); 0.8522 (0.8); 0.8502
(0.9); 0.8449 (2.3); 0.8401 (2.1); 0.8335 (0.6); 0.8293 (0.7); 0.6639 (1.1); 0.6584 (1.0); 0.6563 (1.3); 0.6529 (1.9); 0.6506 (1.7);
0.6479 (1.9); 0.6455 (1.3); 0.6430 (1.4); 0.6403 (2.4); 0.6348 (1.6); 0.6276 (1.0); 0.0079 (0.8); −0.0002 (36.4); −0.0085 (1.1)
III-29: ¹H-NMR(400.6 MHz, CDCl3):
δ = 8.4999 (4.4); 8.4869 (4.5); 8.4140 (3.9); 8.4084 (3.9); 8.1283 (2.4); 8.1220 (2.4); 7.7759 (1.1); 7.7696 (1.1); 7.7574 (1.2);
7.7547 (1.4); 7.7511 (1.3); 7.7484 (1.3); 7.7362 (1.2); 7.7299 (1.1); 7.4249 (2.0); 7.4118 (2.2); 7.4098 (2.4); 7.3965 (2.0); 7.2617
(17.2); 7.0108 (1.7); 7.0033 (1.6); 6.9896 (1.6); 6.9821 (1.5); 5.3001 (3.8); 4.9079 (16.0); 4.3121 (2.0); 4.2943 (6.4); 4.2765 (6.5);
4.2587 (2.1); 1.3248 (7.7); 1.3070 (15.8); 1.2891 (7.6); 0.0079 (0.8); −0.0002 (24.8); −0.0085 (0.7)
I-304: ¹H-NMR(400.6 MHz, CDCl3):
δ = 8.2665 (1.0); 8.2648 (1.0); 8.2550 (1.0); 8.2531 (1.0); 8.1300 (1.4); 8.1238 (1.4); 7.8610 (0.7); 7.8547 (0.7); 7.8422 (0.8);
7.8397 (0.8); 7.8360 (0.8); 7.8335 (0.8); 7.8211 (0.7); 7.8148 (0.7); 7.5190 (0.7); 7.5153 (0.6); 7.4982 (1.0); 7.4948 (1.1); 7.4922
(0.8); 7.4752 (0.9); 7.4715 (0.8); 7.3654 (0.8); 7.3563 (0.9); 7.3538 (0.8); 7.3448 (1.4); 7.3356 (0.7); 7.3332 (0.6); 7.3241 (0.6);
7.2608 (17.8); 6.9702 (0.9); 6.9688 (0.9); 6.9627 (1.0); 6.9489 (0.9); 6.9475 (0.9); 6.9414 (0.9); 4.9586 (9.0); 3.8034 (16.0);
1.5463 (2.6); 0.0079 (0.8); −0.0002 (26.7); −0.0085 (0.7)
I-305: ¹H-NMR(400.6 MHz, CDCl3):
δ = 8.2303 (1.4); 8.2283 (1.4); 8.2263 (1.3); 8.2243 (1.5); 8.2222 (1.4); 8.1028 (1.1); 8.1007 (1.2); 8.0981 (1.3); 8.0960 (1.2);
8.0907 (1.2); 8.0885 (1.3); 8.0860 (1.3); 8.0838 (1.2); 7.8593 (0.6); 7.8578 (0.6); 7.8531 (0.6); 7.8516 (0.6); 7.8404 (0.7); 7.8385
(1.0); 7.8366 (0.8); 7.8343 (0.7); 7.8323 (1.0); 7.8305 (0.7); 7.8193 (0.6); 7.8178 (0.6); 7.8130 (0.6); 7.8116 (0.6); 7.7806 (1.1);
7.7759 (1.1); 7.7623 (1.2); 7.7599 (1.6); 7.7575 (1.2); 7.7552 (1.6); 7.7416 (1.7); 7.7368 (1.6); 7.6570 (1.6); 7.6546 (2.8); 7.6523
(1.7); 7.6363 (1.2); 7.6339 (2.0); 7.6315 (1.1); 7.2623 (9.9); 7.1243 (1.4); 7.1217 (1.4); 7.1121 (1.3); 7.1095 (1.4); 7.1060 (1.4);
7.1033 (1.3); 7.0938 (1.3); 7.0912 (1.3); 6.9890 (1.3); 6.9874 (1.4); 6.9816 (1.3); 6.9799 (1.4); 6.9679 (1.3); 6.9662 (1.3); 6.9604
(1.3); 6.9588 (1.3); 5.3000 (5.4); 4.9336 (14.4); 4.3272 (1.8); 4.3094 (5.7); 4.2916 (5.7); 4.2738 (1.8); 1.5678 (0.7); 1.4399 (0.6);
1.3359 (7.8); 1.3181 (16.0); 1.3132 (0.5); 1.3003 (7.4); −0.0002 (15.4); −0.0027 (0.6)
I-306: ¹H-NMR(400.6 MHz, CDCl3):
δ = 8.2724 (2.3); 8.2704 (2.3); 8.2689 (2.2); 8.2623 (2.1); 8.2607 (2.4); 8.2588 (2.4); 8.0921 (2.8); 8.0861 (2.9); 7.7904 (1.0);
7.7843 (1.0); 7.7710 (1.6); 7.7648 (1.5); 7.7507 (1.1); 7.7455 (1.1); 7.5406 (1.6); 7.5368 (1.6); 7.5197 (2.3); 7.5166 (2.8); 7.5135
(1.8); 7.4965 (2.0); 7.4928 (2.0); 7.3675 (1.9); 7.3585 (2.2); 7.3558 (2.0); 7.3468 (3.4); 7.3378 (1.6); 7.3352 (1.6); 7.3262 (1.4);
7.2606 (86.8); 7.2334 (0.8); 6.9666 (2.1); 6.9652 (2.2); 6.9591 (2.2); 6.9576 (2.2); 6.9454 (2.1); 6.9439 (2.2); 6.9379 (2.2); 6.9364
(2.1); 5.3686 (1.1); 5.3512 (4.7); 5.3337 (4.8); 5.3163 (1.2); 5.3002 (0.6); 1.7431 (16.0); 1.7356 (1.4); 1.7256 (16.0); 1.7182 (1.3);
1.4322 (1.4); 1.2628 (1.3); 0.8991 (0.6); 0.8820 (2.3); 0.8642 (0.9); 0.0080 (3.7); −0.0002 (132.9); −0.0085 (3.9); −0.0272 (1.4)
I-307: ¹H-NMR(400.6 MHz, CDCl3):
δ = 8.1737 (0.8); 8.1718 (0.9); 8.1702 (0.9); 8.1620 (0.8); 8.1602 (0.9); 8.1585 (0.9); 8.1363 (1.3); 8.1301 (1.3); 7.7708 (0.6);
7.7647 (0.6); 7.7517 (0.7); 7.7498 (0.8); 7.7455 (0.7); 7.7437 (0.7); 7.7307 (0.6); 7.7245 (0.6); 7.4651 (0.6); 7.4614 (0.6); 7.4445
(0.8); 7.4411 (1.2); 7.4378 (0.7); 7.4209 (0.8); 7.4172 (0.7); 7.2625 (6.2); 7.2529 (0.8); 7.2502 (0.8); 7.2413 (1.3); 7.2324 (0.6);
7.2296 (0.6); 7.2208 (0.6); 6.9129 (0.8); 6.9118 (0.8); 6.9055 (0.9); 6.8919 (0.8); 6.8906 (0.8); 6.8844 (0.8); 6.8833 (0.8); 5.2726
(1.6); 5.2552 (1.6); 3.7521 (16.0); 1.6366 (6.6); 1.6193 (6.5); 1.5856 (0.6); 1.5783 (1.6); 1.5656 (0.9); 1.5515 (0.5); 1.5442 (0.6);
0.8163 (0.6); 0.8111 (1.4); 0.8066 (0.9); 0.8052 (0.9); 0.8026 (1.0); 0.7979 (1.4); 0.7937 (1.2); 0.7887 (0.6); 0.7801 (1.3); 0.7706
(2.0); 0.7670 (0.9); 0.7631 (0.6); 0.7597 (0.6); 0.7499 (1.8); 0.7455 (0.9); −0.0002 (8.1)
I-308: ¹H-NMR(400.6 MHz, CDCl3):
δ = 8.2251 (1.4); 8.2235 (1.6); 8.2216 (1.6); 8.2200 (1.5); 8.2135 (1.5); 8.2119 (1.7); 8.2100 (1.7); 8.2085 (1.5); 8.1100 (2.0);
8.1039 (2.1); 7.8291 (0.7); 7.8229 (0.7); 7.8088 (1.1); 7.8030 (1.0); 7.7894 (0.8); 7.7832 (0.7); 7.5067 (1.1); 7.5030 (1.1); 7.4859
(1.6); 7.4840 (1.6); 7.4823 (1.8); 7.4805 (1.4); 7.4633 (1.5); 7.4595 (1.5); 7.3658 (1.5); 7.3566 (1.6); 7.3542 (1.6); 7.3450 (2.5);
7.3358 (1.1); 7.3335 (1.2); 7.3243 (1.0); 7.2622 (10.4); 6.9400 (1.5); 6.9387 (1.6); 6.9326 (1.6); 6.9314 (1.6); 6.9189 (1.4); 6.9175
(1.5); 6.9114 (1.5); 6.9101 (1.5); 5.2859 (0.8); 5.2685 (3.0); 5.2512 (3.0); 5.2338 (0.8); 4.2499 (1.0); 4.2447 (1.1); 4.2321 (3.3);
4.2270 (3.4); 4.2142 (3.4); 4.2092 (3.4); 4.1964 (1.1); 4.1915 (1.1); 1.6798 (12.6); 1.6624 (12.5); 1.2647 (7.8); 1.2538 (1.0);
1.2469 (16.0); 1.2291 (7.5); −0.0002 (17.0); −0.0085 (0.6)
I-309: ¹H-NMR(400.6 MHz, CDCl3):
δ = 8.2322 (1.0); 8.2301 (0.9); 8.2279 (1.0); 8.2262 (1.0); 8.1021 (0.7); 8.1001 (0.8); 8.0974 (0.8); 8.0953 (0.8); 8.0900 (0.8);
8.0879 (0.8); 8.0853 (0.8); 8.0832 (0.8); 7.8414 (0.7); 7.8352 (0.6); 7.7920 (0.6); 7.7873 (0.6); 7.7737 (0.7); 7.7713 (0.9); 7.7690
(0.8); 7.7666 (0.9); 7.7530 (0.9); 7.7482 (0.9); 7.6690 (1.0); 7.6666 (1.7); 7.6643 (1.0); 7.6483 (0.7); 7.6460 (1.2); 7.6436 (0.7);
7.2620 (8.8); 7.1283 (0.8); 7.1257 (0.8); 7.1161 (0.8); 7.1135 (0.8); 7.1100 (0.8); 7.1073 (0.8); 7.0978 (0.8); 7.0952 (0.7); 6.9896
(0.8); 6.9882 (0.8); 6.9822 (0.8); 6.9808 (0.8); 6.9684 (0.8); 6.9669 (0.8); 6.9610 (0.8); 6.9596 (0.8); 5.3003 (0.9); 4.9362 (8.5);
4.5272 (2.0); 4.5115 (4.2); 4.4958 (2.1); 3.6877 (16.0); 2.7228 (2.0); 2.7071 (4.0); 2.6914 (2.0); 1.5626 (4.1); −0.0002 (12.9)
I-311: ¹H-NMR(400.6 MHz, CDCl3):
δ = 8.1481 (3.2); 8.1462 (2.4); 8.1439 (2.5); 8.1418 (3.4); 7.8952 (1.7); 7.8889 (1.6); 7.8765 (1.9); 7.8740 (2.0); 7.8703 (1.9);
7.8677 (2.0); 7.8554 (1.8); 7.8491 (1.7); 7.6224 (1.4); 7.6075 (1.6); 7.6022 (1.8); 7.6006 (1.9); 7.5874 (1.8); 7.5858 (1.9); 7.5805
(1.6); 7.5656 (1.5); 7.2650 (1.1); 7.2607 (85.7); 7.0136 (2.1); 7.0121 (2.2); 7.0062 (2.2); 7.0047 (2.2); 6.9972 (0.6); 6.9924 (2.1);
6.9908 (2.2); 6.9849 (2.1); 6.9834 (2.1); 6.9546 (1.6); 6.9483 (1.8); 6.9454 (1.8); 6.9391 (1.7); 6.9327 (1.5); 6.9265 (1.6); 6.9235
(1.6); 6.9172 (1.4); 5.3541 (1.1); 5.3367 (4.6); 5.3192 (4.7); 5.3017 (1.2); 1.7544 (16.0); 1.7369 (16.0); 1.2645 (0.9); 0.8820 (1.8);
0.8643 (0.7); 0.0690 (1.6); 0.0080 (2.9); 0.0041 (1.0); −0.0002 (118.3); −0.0085 (3.5)
I-312: ¹H-NMR(400.6 MHz, CDCl3):
δ = 8.2221 (1.9); 8.2202 (1.5); 8.2181 (1.5); 8.2159 (2.0); 8.2142 (1.4); 7.8801 (1.1); 7.8740 (1.1); 7.8612 (1.3); 7.8591 (1.4);
7.8550 (1.2); 7.8529 (1.3); 7.8401 (1.2); 7.8339 (1.1); 7.6363 (1.7); 7.6165 (2.7); 7.5972 (2.2); 7.4238 (2.3); 7.4035 (1.9); 7.2623
(10.2); 6.9962 (1.4); 6.9947 (1.5); 6.9888 (1.4); 6.9873 (1.5); 6.9751 (1.3); 6.9736 (1.4); 6.9677 (1.3); 6.9662 (1.4); 6.9483 (2.1);
6.9295 (2.0); 5.3002 (3.4); 4.9423 (15.1); 4.3220 (1.9); 4.3042 (5.9); 4.2864 (6.0); 4.2686 (2.0); 2.1561 (16.0); 1.5706 (4.8);
1.5017 (0.9); 1.3332 (7.6); 1.3154 (15.7); 1.2975 (7.4); −0.0002 (14.5)
I-313: ¹H-NMR(400.6 MHz, CDCl3):
δ = 8.1976 (2.2); 8.1914 (2.3); 8.1202 (1.3); 8.1175 (1.4); 8.1082 (1.4); 8.1054 (1.4); 7.8461 (1.0); 7.8399 (1.0); 7.8251 (1.3);
7.8213 (1.2); 7.8190 (1.2); 7.8062 (1.1); 7.8004 (1.5); 7.7968 (1.1); 7.7812 (1.5); 7.7763 (1.4); 7.7625 (1.3); 7.7577 (1.3); 7.5901
(2.6); 7.5696 (2.2); 7.5189 (0.5); 7.2604 (97.6); 7.1596 (1.3); 7.1474 (1.3); 7.1434 (1.2); 7.1410 (1.2); 7.1313 (1.2); 7.0035 (1.5);
6.9968 (1.9); 6.9823 (1.4); 6.9750 (1.4); 5.3002 (7.3); 4.9975 (14.0); 4.6975 (3.2); 4.6837 (3.9); 4.6694 (3.3); 3.3760 (2.0); 3.3618
(3.2); 3.3480 (1.9); 2.9716 (16.0); 1.6302 (1.9); 1.2549 (1.2); 0.1456 (0.5); 0.0130 (0.6); 0.0080 (4.0); −0.0002 (149.0); −0.0085
(4.6); −0.1497 (0.6)
III-30: ¹H-NMR(400.6 MHz, d₆-DMSO):
δ = 13.1222 (0.9); 8.3790 (3.8); 8.3727 (3.9); 8.3280 (6.1); 8.3269 (6.0); 8.3140 (6.4); 8.3129 (6.3); 8.1177 (1.6); 8.1114 (1.5);
8.0982 (2.0); 8.0964 (2.2); 8.0920 (2.0); 8.0902 (2.0); 8.0771 (1.7); 8.0708 (1.6); 7.4236 (2.2); 7.4224 (2.3); 7.4169 (2.3); 7.4156
(2.2); 7.4024 (2.1); 7.4011 (2.2); 7.3956 (2.2); 7.3942 (2.0); 7.3187 (5.7); 7.3176 (6.4); 7.3139 (6.5); 7.3127 (6.1); 7.0230 (5.4);
7.0181 (4.9); 7.0091 (5.2); 7.0041 (5.1); 5.1986 (1.0); 5.1813 (5.3); 5.1639 (5.4); 5.1466 (1.0); 3.6216 (1.0); 3.6177 (5.5); 3.6155
(3.1); 3.6115 (3.0); 3.6093 (2.2); 3.6075 (3.9); 3.6029 (4.3); 3.6011 (12.9); 3.5989 (4.4); 3.5950 (4.1); 3.5930 (2.2); 3.5908 (2.8);
3.5867 (2.9); 3.5845 (5.9); 3.5806 (1.0); 3.3226 (15.0); 2.6705 (0.5); 2.5242 (1.6); 2.5196 (2.2); 2.5109 (27.2); 2.5063 (58.7);
2.5017 (81.7); 2.4971 (55.4); 2.4925 (23.8); 2.1831 (0.8); 1.9088 (1.1); 1.7809 (0.9); 1.7780 (1.0); 1.7762 (5.7); 1.7746 (2.4);
1.7686 (4.5); 1.7671 (3.5); 1.7643 (2.7); 1.7597 (16.0); 1.7550 (2.8); 1.7507 (4.4); 1.7447 (2.2); 1.7431 (5.5); 1.7385 (0.8); 1.5997
(13.6); 1.5823 (13.4); 1.3554 (7.7); 1.2390 (0.7); 1.2350 (0.9); 0.0080 (2.7); −0.0002 (102.8); −0.0060 (0.6); −0.0069 (0.5); −0.0085 (2.8)
I-315: ¹H-NMR(400.6 MHz, CDCl3):
δ = 8.1466 (2.0); 8.1404 (2.1); 7.8929 (1.1); 7.8866 (1.0); 7.8742 (1.2); 7.8717 (1.3); 7.8680 (1.2); 7.8654 (1.2); 7.8530 (1.2);
7.8468 (1.1); 7.6205 (1.0); 7.6056 (1.0); 7.6003 (1.2); 7.5988 (1.3); 7.5854 (1.3); 7.5840 (1.3); 7.5787 (1.1); 7.5638 (1.1); 7.2614
(24.6); 7.0106 (1.4); 7.0094 (1.5); 7.0032 (1.5); 7.0020 (1.4); 6.9895 (1.4); 6.9881 (1.4); 6.9820 (1.4); 6.9580 (1.1); 6.9518 (1.2);
6.9488 (1.2); 6.9425 (1.1); 6.9362 (1.0); 6.9299 (1.1); 6.9270 (1.1); 6.9207 (1.0); 4.9244 (16.0); 4.3006 (2.0); 4.2827 (6.1); 4.2649
(6.2); 4.2471 (2.0); 1.5505 (13.2); 1.3146 (7.4); 1.2967 (15.2); 1.2789 (7.3); 0.0079 (0.9); −0.0002 (33.1); −0.0085 (0.9)
I-316: ¹H-NMR(400.6 MHz, CDCl3):
δ = 8.2295 (1.1); 8.2282 (1.2); 8.2251 (1.3); 8.2237 (1.2); 8.2177 (1.2); 8.2163 (1.2); 8.2132 (1.2); 8.2118 (1.1); 8.1024 (1.3);
8.1005 (1.9); 8.0983 (1.5); 8.0962 (1.5); 8.0942 (2.0); 8.0922 (1.3); 7.8406 (1.2); 7.8343 (1.1); 7.8218 (1.3); 7.8193 (1.4); 7.8156
(1.2); 7.8130 (1.3); 7.8006 (1.2); 7.7943 (1.2); 7.6387 (1.1); 7.6369 (1.2); 7.6343 (1.2); 7.6325 (1.1); 7.6197 (1.2); 7.6178 (1.4);
7.6153 (1.3); 7.6134 (1.2); 7.2629 (10.1); 7.2458 (1.7); 7.2339 (1.6); 7.2268 (1.6); 7.2149 (1.5); 6.9238 (1.4); 6.9223 (1.4); 6.9163
(1.4); 6.9148 (1.4); 6.9026 (1.4); 6.9010 (1.3); 6.8951 (1.4); 6.8935 (1.3); 4.8987 (15.0); 4.2792 (1.9); 4.2613 (6.0); 4.2435 (6.0);
4.2257 (2.0); 2.1822 (13.7); 1.5768 (5.4); 1.3030 (7.6); 1.2852 (16.0); 1.2673 (7.6); 0.0701 (0.8); −0.0002 (14.9)
I-317: ¹H-NMR(400.6 MHz, CDCl3):
δ = 8.2587 (2.1); 8.2571 (2.4); 8.2551 (2.4); 8.2471 (2.3); 8.2455 (2.5); 8.2435 (2.5); 8.1152 (3.1); 8.1091 (3.2); 7.8398 (1.0);
7.8335 (1.0); 7.8198 (1.5); 7.8134 (1.4); 7.8001 (1.0); 7.7940 (1.0); 7.5146 (1.6); 7.5109 (1.7); 7.4938 (2.5); 7.4922 (2.3); 7.4901
(2.6); 7.4886 (2.1); 7.4713 (2.3); 7.4676 (2.3); 7.3875 (2.2); 7.3783 (2.5); 7.3759 (2.3); 7.3667 (3.7); 7.3575 (1.6); 7.3552 (1.7);
7.3459 (1.5); 7.2608 (50.0); 6.9525 (2.2); 6.9511 (2.3); 6.9453 (2.4); 6.9438 (2.3); 6.9314 (2.2); 6.9299 (2.2); 6.9240 (2.2); 6.9225
(2.2); 5.3708 (1.1); 5.3534 (4.2); 5.3359 (4.3); 5.3185 (1.2); 5.3002 (11.9); 1.7345 (16.0); 1.7170 (16.0); 1.7047 (0.9); 1.2540 (0.9);
0.0079 (2.4); −0.0002 (78.1); −0.0085 (2.2)
I-318: ¹H-NMR(400.6 MHz, CDCl3):
δ = 8.2287 (0.7); 8.2268 (0.9); 8.2251 (0.9); 8.2232 (0.8); 8.2171 (0.8); 8.2152 (0.9); 8.2134 (0.9); 8.2116 (0.8); 8.1362 (1.2);
8.1341 (0.9); 8.1320 (0.9); 8.1299 (1.2); 7.7748 (0.6); 7.7685 (0.6); 7.7557 (0.7); 7.7537 (0.8); 7.7495 (0.7); 7.7475 (0.8); 7.7347
(0.7); 7.7285 (0.6); 7.4615 (0.6); 7.4578 (0.7); 7.4409 (0.8); 7.4375 (1.1); 7.4344 (0.7); 7.4175 (0.8); 7.4138 (0.8); 7.2861 (0.8);
7.2772 (0.9); 7.2745 (0.8); 7.2655 (1.7); 7.2614 (27.8); 7.2568 (0.9); 7.2539 (0.8); 7.2450 (0.6); 6.9202 (0.8); 6.9188 (0.8); 6.9128
(0.8); 6.9113 (0.8); 6.8992 (0.8); 6.8976 (0.8); 6.8917 (0.8); 6.8902 (0.8); 4.9141 (8.9); 4.0314 (3.8); 4.0132 (3.9); 1.5972 (0.6);
1.5833 (1.1); 1.5685 (0.6); 1.5581 (12.9); 1.5563 (16.0); 1.5553 (15.3); 1.1408 (0.7); 0.8038 (0.8); 0.8011 (1.0); 0.7987 (2.0);
0.7949 (1.6); 0.7889 (2.0); 0.7850 (3.6); 0.7804 (2.0); 0.7694 (1.3); 0.7648 (1.7); 0.7617 (0.9); 0.7588 (1.0); 0.5559 (1.3); 0.5527
(1.6); 0.5503 (0.9); 0.5481 (0.6); 0.5409 (0.6); 0.5358 (1.5); 0.5325 (1.2); 0.5209 (0.6); 0.2972 (0.6); 0.2856 (1.7); 0.2825 (1.4);
0.2737 (1.3); 0.2705 (1.7); 0.0080 (1.4); 0.0049 (0.5); −0.0002 (46.4); −0.0085 (1.2)
I-320: ¹H-NMR(400.6 MHz, CDCl3):
δ = 8.2211 (2.1); 8.2112 (2.1); 8.0896 (2.6); 8.0839 (2.6); 7.7890 (0.9); 7.7835 (0.9); 7.7694 (1.5); 7.7633 (1.5); 7.7497 (1.0);
7.7436 (0.9); 7.5421 (1.2); 7.5384 (1.2); 7.5213 (1.8); 7.5183 (2.5); 7.4980 (1.5); 7.4943 (1.4); 7.3462 (1.3); 7.3373 (1.6); 7.3347
(1.5); 7.3257 (2.4); 7.3166 (1.3); 7.3142 (1.2); 7.3051 (1.0); 7.2610 (35.4); 6.9561 (1.9); 6.9487 (1.9); 6.9340 (1.8); 6.9275 (1.8);
5.2684 (1.0); 5.2510 (3.3); 5.2336 (3.4); 5.2163 (1.0); 4.2602 (2.2); 4.2423 (6.9); 4.2245 (7.1); 4.2067 (2.4); 1.6867 (13.3); 1.6693
(13.2); 1.5525 (12.1); 1.2765 (7.8); 1.2587 (16.0); 1.2409 (7.6); 0.0275 (0.5); 0.0079 (2.3); −0.0002 (51.2); −0.0085 (1.9)
I-321: ¹H-NMR(400.6 MHz, CDCl3):
δ = 8.2227 (1.0); 8.2213 (1.1); 8.2182 (1.2); 8.2168 (1.1); 8.2109 (1.1); 8.2094 (1.2); 8.2064 (1.2); 8.2050 (1.1); 8.1167 (1.2);
8.1147 (1.8); 8.1126 (1.4); 8.1105 (1.5); 8.1084 (1.9); 8.1064 (1.3); 7.8530 (1.1); 7.8467 (1.1); 7.8342 (1.2); 7.8317 (1.3); 7.8279
(1.2); 7.8254 (1.3); 7.8129 (1.2); 7.8067 (1.2); 7.6302 (1.0); 7.6284 (1.1); 7.6257 (1.2); 7.6239 (1.1); 7.6111 (1.1); 7.6093 (1.3);
7.6067 (1.3); 7.6048 (1.2); 7.2627 (12.1); 7.2374 (1.6); 7.2255 (1.5); 7.2184 (1.4); 7.2065 (1.4); 6.9198 (1.3); 6.9182 (1.4); 6.9123
(1.4); 6.9107 (1.4); 6.8985 (1.3); 6.8969 (1.4); 6.8910 (1.3); 6.8894 (1.3); 5.3003 (2.9); 4.8974 (14.1); 4.2761 (1.8); 4.2583 (5.8);
4.2405 (5.8); 4.2226 (1.9); 2.1766 (12.5); 1.5748 (3.0); 1.3015 (7.6); 1.2837 (16.0); 1.2658 (7.5); 0.0699 (1.0); −0.0002
(17.5); −0.0085 (0.5)
I-322: ¹H-NMR(400.6 MHz, CDCl3):
δ = 8.2087 (0.9); 8.2024 (1.0); 8.0596 (0.6); 8.0570 (0.6); 8.0549 (0.6); 8.0495 (0.6); 8.0475 (0.7); 8.0448 (0.7); 8.0428 (0.6);
7.8449 (0.5); 7.8428 (0.6); 7.8388 (0.6); 7.8366 (0.6); 7.8239 (0.5); 7.8177 (0.5); 7.7501 (0.6); 7.7479 (0.7); 7.7454 (0.6); 7.7432
(0.7); 7.7294 (0.7); 7.7247 (0.7); 7.6106 (1.3); 7.6084 (0.8); 7.5923 (0.6); 7.5900 (1.0); 7.2603 (30.9); 7.1124 (0.6); 7.1099 (0.6);
7.1003 (0.6); 7.0978 (0.6); 7.0941 (0.6); 7.0915 (0.6); 7.0819 (0.6); 7.0794 (0.6); 6.9961 (0.7); 6.9872 (0.7); 6.9733 (0.7); 6.9661
(0.6); 6.2991 (1.7); 6.2967 (1.8); 5.8631 (0.6); 5.8595 (1.4); 5.8569 (1.3); 5.8532 (0.6); 5.3044 (1.3); 5.2999 (2.4); 5.2870 (1.3);
4.9341 (2.8); 3.7478 (14.2); 1.7412 (4.5); 1.7237 (4.5); 1.5400 (16.0); 0.0079 (1.4); 0.0055 (0.5); −0.0002 (46.2); −0.0085 (1.3)
I-323: ¹H-NMR(400.6 MHz, d₆-DMSO):
δ = 8.1910 (1.1); 8.1895 (1.1); 8.1865 (1.2); 8.1851 (1.2); 8.1791 (1.2); 8.1776 (1.2); 8.1747 (1.2); 8.1732 (1.2); 8.1632 (1.8);
8.1613 (1.3); 8.1589 (1.4); 8.1569 (1.8); 7.9250 (0.8); 7.9187 (0.8); 7.9056 (1.1); 7.9037 (1.1); 7.8993 (1.0); 7.8974 (1.1); 7.8844
(1.0); 7.8778 (1.8); 7.8759 (1.2); 7.8732 (1.2); 7.8714 (1.0); 7.8586 (1.1); 7.8567 (1.3); 7.8542 (1.2); 7.8523 (1.0); 7.4040 (1.5);
7.3921 (1.5); 7.3852 (1.4); 7.3733 (1.4); 7.2611 (1.1); 7.2598 (1.2); 7.2542 (1.2); 7.2528 (1.1); 7.2399 (1.1); 7.2384 (1.2); 7.2328
(1.1); 7.2314 (1.1); 5.7570 (16.0); 4.8449 (8.5); 3.3238 (2.8); 2.5246 (0.8); 2.5200 (1.1); 2.5113 (14.5); 2.5067 (31.3); 2.5021
(43.6); 2.4975 (29.4); 2.4929 (12.6); 2.2167 (10.8); 1.9890 (0.5); 0.0080 (1.2); −0.0002 (39.2); −0.0085 (1.0)
I-324: ¹H-NMR(400.6 MHz, d₆-DMSO):
δ = 8.1821 (1.5); 8.1806 (1.6); 8.1776 (1.8); 8.1761 (1.7); 8.1703 (1.8); 8.1688 (1.9); 8.1639 (3.7); 8.1588 (2.1); 8.1568 (2.8);
7.9280 (1.2); 7.9217 (1.2); 7.9086 (1.5); 7.9068 (1.6); 7.9023 (1.5); 7.9005 (1.6); 7.8874 (1.3); 7.8811 (1.3); 7.8674 (1.5); 7.8656
(1.6); 7.8630 (1.8); 7.8612 (1.5); 7.8484 (1.6); 7.8466 (1.9); 7.8440 (1.8); 7.8421 (1.6); 7.3922 (2.3); 7.3804 (2.2); 7.3735 (2.2);
7.3616 (2.1); 7.2551 (1.6); 7.2538 (1.7); 7.2482 (1.8); 7.2469 (1.7); 7.2339 (1.6); 7.2325 (1.7); 7.2269 (1.7); 7.2255 (1.6); 5.7572
(4.7); 4.8287 (11.9); 4.0383 (0.8); 4.0206 (0.8); 3.3273 (0.9); 2.5418 (0.6); 2.5251 (0.8); 2.5204 (1.0); 2.5117 (15.1); 2.5071 (33.0);
2.5025 (46.2); 2.4979 (31.4); 2.4933 (13.6); 2.2127 (16.0); 1.9892 (4.0); 1.1924 (1.2); 1.1746 (2.4); 1.1569 (1.2); 0.0081
(1.0); −0.0002 (37.2); −0.0085 (1.0)
I-325: ¹H-NMR(400.6 MHz, CDCl3):
δ = 8.4912 (1.4); 8.4870 (1.5); 8.4792 (1.5); 8.4750 (1.5); 8.1544 (1.5); 8.1500 (1.6); 8.1346 (1.7); 8.1302 (1.7); 8.1202 (2.2);
8.1185 (1.8); 8.1162 (1.8); 8.1140 (2.3); 7.8567 (1.1); 7.8505 (1.1); 7.8380 (1.3); 7.8355 (1.4); 7.8318 (1.3); 7.8293 (1.3); 7.8168
(1.2); 7.8105 (1.2); 7.4626 (1.1); 7.4610 (1.2); 7.4506 (1.1); 7.4490 (1.2); 7.4429 (1.1); 7.4413 (1.2); 7.4308 (1.0); 7.4293 (1.1);
7.2620 (12.0); 6.9576 (1.4); 6.9563 (1.6); 6.9502 (1.5); 6.9488 (1.6); 6.9364 (1.4); 6.9350 (1.6); 6.9289 (1.4); 6.9275 (1.6); 4.8784
(16.0); 4.2818 (2.0); 4.2639 (6.2); 4.2461 (6.3); 4.2282 (2.0); 1.5618 (7.9); 1.5033 (2.5); 1.3006 (7.8); 1.2828 (16.0); 1.2649 (9.0);
0.8986 (0.9); 0.8818 (3.1); 0.8640 (1.2); −0.0002 (17.8); −0.0085 (0.6)
I-328: ¹H-NMR(400.6 MHz, CDCl3):
δ = 8.2210 (0.7); 8.2190 (0.8); 8.2173 (0.8); 8.2155 (0.7); 8.2093 (0.7); 8.2074 (0.8); 8.2056 (0.8); 8.2038 (0.7); 8.1357 (1.1);
8.1295 (1.1); 7.7744 (0.6); 7.7682 (0.6); 7.7554 (0.6); 7.7534 (0.7); 7.7492 (0.6); 7.7472 (0.7); 7.7343 (0.6); 7.7282 (0.6); 7.4628
(0.6); 7.4591 (0.6); 7.4422 (0.8); 7.4388 (1.1); 7.4357 (0.7); 7.4188 (0.8); 7.4151 (0.8); 7.2832 (0.7); 7.2743 (0.8); 7.2716 (0.8);
7.2624 (13.9); 7.2537 (0.6); 7.2510 (0.6); 7.2421 (0.6); 6.9192 (0.7); 6.9177 (0.8); 6.9118 (0.8); 6.9103 (0.8); 6.8981 (0.7); 6.8966
(0.8); 6.8906 (0.7); 6.8891 (0.7); 5.1308 (0.9); 5.1151 (1.3); 5.0995 (0.9); 4.8507 (9.6); 1.5937 (0.5); 1.5798 (2.3); 1.5722 (5.8);
1.5596 (0.7); 1.2653 (16.0); 1.2497 (15.9); 0.8068 (0.7); 0.8013 (1.8); 0.7971 (1.3); 0.7938 (0.9); 0.7891 (2.2); 0.7852 (2.0);
0.7829 (2.0); 0.7798 (0.8); 0.7693 (1.2); 0.7638 (1.4); 0.7614 (0.8); 0.7583 (0.9); −0.0002 (18.2)
I-329: ¹H-NMR(400.6 MHz, d₆-DMSO):
δ = 8.1146 (1.8); 8.1109 (2.0); 8.1032 (2.0); 8.0995 (2.0); 8.0844 (1.5); 8.0800 (1.1); 8.0781 (1.5); 7.8629 (0.7); 7.8566 (0.7);
7.8436 (0.9); 7.8417 (0.9); 7.8373 (0.8); 7.8353 (0.9); 7.8224 (0.8); 7.8160 (0.7); 7.5998 (1.0); 7.5961 (1.1); 7.5787 (2.0); 7.5750
(1.8); 7.5391 (2.5); 7.5277 (2.3); 7.5182 (1.3); 7.5068 (1.4); 7.2634 (1.0); 7.2620 (1.0); 7.2564 (1.0); 7.2552 (0.9); 7.2421 (0.9);
7.2406 (1.0); 7.2351 (0.9); 7.2337 (0.9); 5.7571 (6.0); 4.8243 (7.4); 3.5793 (16.0); 3.3237 (0.8); 2.5248 (0.6); 2.5201 (0.7); 2.5114
(8.8); 2.5068 (18.8); 2.5022 (26.1); 2.4976 (17.6); 2.4931 (7.5); 0.0079 (0.9); −0.0002 (33.1); −0.0027 (1.1); −0.0085 (0.9)
I-331: ¹H-NMR(400.6 MHz, CDCl3):
δ = 8.2191 (0.9); 8.2172 (1.0); 8.2074 (0.9); 8.2056 (1.0); 8.1132 (1.2); 8.1072 (1.2); 7.8114 (0.6); 7.8056 (0.6); 7.5110 (0.6);
7.5073 (0.6); 7.4901 (0.9); 7.4866 (1.0); 7.4675 (0.8); 7.4638 (0.8); 7.3660 (0.8); 7.3568 (0.9); 7.3545 (0.8); 7.3453 (1.3); 7.3360
(0.6); 7.3337 (0.6); 7.3245 (0.6); 7.2607 (13.4); 6.9399 (0.9); 6.9327 (0.9); 6.9179 (0.8); 6.9114 (0.8); 5.2861 (1.5); 5.2687 (1.5);
3.7646 (16.0); 1.6841 (6.6); 1.6667 (6.6); 1.5454 (4.0); 0.0080 (0.6); −0.0002 (19.6); −0.0085 (0.5)
I-332: ¹H-NMR(400.6 MHz, CDCl3):
δ = 8.1469 (0.7); 8.1449 (1.0); 8.1429 (0.8); 8.1408 (0.8); 8.1387 (1.0); 8.1368 (0.7); 7.8898 (0.6); 7.8835 (0.6); 7.8711 (0.7);
7.8685 (0.7); 7.8649 (0.7); 7.8623 (0.7); 7.8499 (0.6); 7.8436 (0.6); 7.6223 (0.5); 7.6074 (0.6); 7.6020 (0.6); 7.6005 (0.7); 7.5871
(0.7); 7.5856 (0.7); 7.5803 (0.6); 7.5654 (0.6); 7.2623 (7.6); 7.0064 (0.7); 7.0048 (0.8); 6.9989 (0.8); 6.9974 (0.7); 6.9852 (0.7);
6.9836 (0.7); 6.9777 (0.7); 6.9761 (0.7); 6.9388 (0.6); 6.9325 (0.7); 6.9294 (0.6); 6.9232 (0.6); 6.9169 (0.5); 6.9107 (0.6); 6.9076
(0.6); 6.9014 (0.5); 5.2588 (1.5); 5.2414 (1.5); 3.7799 (16.0); 1.7012 (6.4); 1.6838 (6.3); 1.5616 (3.1); −0.0002 (10.7)
I-333: ¹H-NMR(400.6 MHz, CDCl3):
δ = 8.1523 (3.2); 8.1460 (3.3); 7.9004 (1.7); 7.8941 (1.6); 7.8818 (1.9); 7.8792 (2.0); 7.8755 (1.8); 7.8730 (1.9); 7.8606 (1.8);
7.8543 (1.6); 7.6160 (1.5); 7.6012 (1.6); 7.5960 (1.8); 7.5943 (1.8); 7.5811 (1.9); 7.5794 (1.8); 7.5742 (1.6); 7.5594 (1.5); 7.2607
(81.4); 7.0094 (2.1); 7.0079 (2.2); 7.0020 (2.2); 7.0005 (2.2); 6.9972 (0.6); 6.9881 (2.1); 6.9866 (2.2); 6.9808 (2.1); 6.9792 (2.0);
6.9507 (1.6); 6.9444 (1.8); 6.9415 (1.8); 6.9352 (1.7); 6.9288 (1.5); 6.9225 (1.6); 6.9196 (1.6); 6.9133 (1.4); 5.3501 (1.1); 5.3326
(4.5); 5.3151 (4.7); 5.3003 (1.1); 5.2977 (1.3); 1.7528 (16.0); 1.7354 (16.0); 1.2645 (2.2); 0.8989 (1.2); 0.8820 (4.4); 0.8643 (1.7);
0.0079 (2.7); 0.0062 (0.6); 0.0053 (0.6); 0.0045 (0.8); −0.0002 (109.9); −0.0053 (1.4); −0.0061 (1.1); −0.0070 (1.0); −0.0085
(3.2); −0.0277 (0.6)
I-334: ¹H-NMR(400.6 MHz, CDCl3):
δ = 8.1553 (2.1); 8.1491 (2.2); 7.9075 (1.1); 7.9013 (1.0); 7.8890 (1.3); 7.8863 (1.4); 7.8827 (1.2); 7.8801 (1.3); 7.8678 (1.2);
7.8615 (1.2); 7.6221 (1.0); 7.6074 (1.1); 7.6022 (1.2); 7.6003 (1.2); 7.5874 (1.2); 7.5855 (1.2); 7.5804 (1.2); 7.5656 (1.0); 7.2606
(87.4); 7.0157 (1.5); 7.0142 (1.5); 7.0083 (1.5); 7.0068 (1.4); 6.9968 (0.6); 6.9944 (1.5); 6.9929 (1.5); 6.9871 (1.5); 6.9855 (1.4);
6.9706 (1.1); 6.9642 (1.3); 6.9614 (1.2); 6.9551 (1.2); 6.9487 (1.0); 6.9424 (1.1); 6.9395 (1.1); 6.9332 (1.0); 5.3003 (1.4); 4.9971
(16.0); 1.8570 (0.6); 1.4322 (4.0); 1.2541 (1.0); 0.0080 (3.2); −0.0002 (119.1); −0.0085 (3.5)
I-335: ¹H-NMR(400.6 MHz, CDCl3):
δ = 8.4874 (1.3); 8.4836 (1.4); 8.4755 (1.4); 8.4717 (1.4); 8.1464 (1.5); 8.1422 (1.6); 8.1322 (2.2); 8.1301 (1.8); 8.1262 (3.7);
8.1239 (2.6); 7.8643 (1.2); 7.8580 (1.1); 7.8455 (1.3); 7.8431 (1.4); 7.8393 (1.3); 7.8369 (1.3); 7.8243 (1.2); 7.8181 (1.2); 7.4570
(1.1); 7.4553 (1.2); 7.4450 (1.1); 7.4433 (1.2); 7.4373 (1.1); 7.4356 (1.1); 7.4252 (1.0); 7.4236 (1.0); 7.2621 (11.3); 6.9530 (1.5);
6.9516 (1.6); 6.9457 (1.5); 6.9442 (1.6); 6.9318 (1.4); 6.9304 (1.5); 6.9244 (1.5); 6.9229 (1.5); 5.3004 (2.6); 4.8774 (15.8); 4.2787
(2.0); 4.2608 (6.3); 4.2430 (6.4); 4.2252 (2.1); 1.5606 (7.6); 1.2988 (7.7); 1.2810 (16.0); 1.2631 (7.6); 0.0699 (1.0); 0.0079
(0.5); −0.0002 (17.6); −0.0085 (0.5)
I-336: ¹H-NMR(400.0 MHz, CDCl3):
δ = 8.1456 (1.1); 8.1396 (1.1); 7.8928 (0.5); 7.8866 (0.5); 7.8717 (0.7); 7.8678 (0.6); 7.8530 (0.5); 7.8464 (0.5); 7.5922 (0.5);
7.5857 (0.7); 7.5712 (0.7); 7.2606 (19.1); 6.9989 (0.8); 6.9916 (0.8); 6.9778 (0.7); 6.9707 (0.7); 6.9294 (0.5); 6.9234 (0.6); 6.9203
(0.6); 6.9142 (0.5); 6.9016 (0.5); 6.8985 (0.5); 5.2298 (1.4); 5.2123 (1.4); 4.2639 (1.0); 4.2461 (3.1); 4.2283 (3.1); 4.2104 (1.0);
1.6948 (5.5); 1.6774 (5.4); 1.5440 (16.0); 1.2864 (3.3); 1.2686 (6.6); 1.2508 (3.2); 0.0078 (1.0); −0.0002 (24.9); −0.0086 (0.8)
I-337: ¹H-NMR(400.6 MHz, CDCl3):
δ = 8.1510 (2.2); 8.1491 (1.7); 8.1468 (1.7); 8.1447 (2.3); 7.9048 (1.2); 7.8986 (1.2); 7.8863 (1.4); 7.8836 (1.5); 7.8800 (1.3);
7.8773 (1.4); 7.8650 (1.3); 7.8587 (1.2); 7.6271 (1.1); 7.6122 (1.1); 7.6071 (1.2); 7.6053 (1.3); 7.5923 (1.3); 7.5905 (1.3); 7.5853
(1.2); 7.5705 (1.1); 7.2607 (55.0); 7.0205 (1.5); 7.0189 (1.6); 7.0131 (1.6); 7.0115 (1.6); 6.9992 (1.5); 6.9975 (1.7); 6.9918 (1.6);
6.9902 (1.5); 6.9739 (1.2); 6.9676 (1.3); 6.9647 (1.3); 6.9584 (1.2); 6.9520 (1.1); 6.9457 (1.2); 6.9428 (1.1); 6.9365 (1.0); 5.3003
(0.7); 5.0011 (16.0); 0.0080 (1.8); 0.0049 (0.5); −0.0002 (73.2); −0.0085 (2.0)
I-338: ¹H-NMR(400.6 MHz, d₆-DMSO):
δ = 12.6158 (1.1); 8.4721 (1.6); 8.4574 (3.5); 8.4426 (1.7); 8.2583 (4.6); 8.2522 (4.8); 8.1001 (2.8); 8.0982 (3.1); 8.0956 (3.4);
8.0935 (3.1); 8.0881 (3.0); 8.0861 (3.3); 8.0834 (3.4); 8.0815 (3.0); 8.0271 (2.0); 8.0209 (1.9); 8.0061 (2.9); 7.9999 (2.8); 7.9864
(2.1); 7.9802 (2.1); 7.9705 (2.3); 7.9658 (2.2); 7.9519 (2.8); 7.9498 (3.2); 7.9473 (2.8); 7.9452 (3.1); 7.9314 (3.1); 7.9267 (2.9);
7.7925 (3.6); 7.7903 (6.2); 7.7883 (3.6); 7.7719 (3.0); 7.7697 (5.0); 7.7676 (2.8); 7.3070 (3.0); 7.3010 (3.0); 7.2867 (5.7); 7.2798
(3.1); 7.2746 (3.0); 7.2721 (3.1); 7.2683 (3.0); 7.2658 (2.7); 7.2562 (2.9); 7.2537 (2.7); 6.8708 (0.9); 6.6408 (0.5); 5.2294 (1.2);
5.2126 (5.2); 5.1957 (5.3); 5.1789 (1.2); 3.8901 (1.3); 3.8748 (1.4); 3.8463 (4.4); 3.8312 (4.1); 3.8175 (4.0); 3.8030 (4.4); 3.7738
(1.4); 3.7595 (1.3); 3.6217 (1.0); 3.6178 (5.0); 3.6155 (3.2); 3.6115 (2.9); 3.6074 (4.0); 3.6011 (12.2); 3.5951 (4.2); 3.5909 (2.9);
3.5867 (3.2); 3.5846 (5.2); 3.5806 (1.0); 3.3228 (14.6); 3.2025 (1.4); 2.6752 (0.5); 2.6707 (0.7); 2.6660 (0.5); 2.5244 (1.9); 2.5197
(2.6); 2.5109 (39.2); 2.5064 (84.6); 2.5018 (116.8); 2.4973 (80.8); 2.4927 (35.9); 2.3290 (0.7); 2.1831 (1.4); 1.9950 (1.2); 1.7806
(1.0); 1.7761 (5.1); 1.7684 (4.7); 1.7642 (3.1); 1.7595 (15.0); 1.7547 (3.2); 1.7507 (4.7); 1.7430 (5.0); 1.7385 (0.9); 1.5517 (16.0);
1.5348 (15.9); 1.3555 (11.8); 1.2388 (1.2); 1.0898 (0.6); 1.0744 (0.6); 0.8540 (0.5); 0.0080 (1.0); −0.0002 (33.9); −0.0085 (1.0)
I-341: ¹H-NMR(400.6 MHz, CDCl3):
δ = 8.2034 (0.9); 8.2016 (0.9); 8.2000 (0.9); 8.1917 (0.9); 8.1900 (0.9); 8.1884 (0.8); 8.1337 (1.2); 8.1275 (1.3); 7.8519 (0.6);
7.8456 (0.6); 7.8331 (0.7); 7.8308 (0.8); 7.8269 (0.7); 7.8246 (0.7); 7.8120 (0.6); 7.8058 (0.6); 7.4998 (0.6); 7.4961 (0.6); 7.4791
(0.8); 7.4757 (1.1); 7.4730 (0.7); 7.4559 (0.8); 7.4522 (0.7); 7.3252 (0.7); 7.3162 (0.8); 7.3136 (0.8); 7.3046 (1.2); 7.2955 (0.6);
7.2930 (0.6); 7.2839 (0.5); 7.2629 (6.5); 6.9545 (0.9); 6.9484 (0.8); 6.9471 (0.9); 6.9346 (0.8); 6.9333 (0.8); 6.9272 (0.8); 6.9259
(0.8); 5.2762 (1.6); 5.2588 (1.6); 3.7655 (16.0); 2.0454 (1.5); 1.6983 (0.8); 1.6939 (6.6); 1.6809 (0.9); 1.6765 (6.5); 1.5801 (4.2);
1.2595 (0.9); −0.0002 (10.6)
I-342: ¹H-NMR(400.6 MHz, CDCl3):
δ = 8.2360 (3.1); 8.2342 (3.2); 8.2327 (2.8); 8.2243 (3.2); 8.2225 (3.1); 8.1353 (4.3); 8.1293 (4.4); 7.7673 (1.7); 7.7611 (1.6);
7.7481 (2.2); 7.7464 (2.6); 7.7421 (2.2); 7.7402 (2.4); 7.7273 (1.8); 7.7211 (1.7); 7.4566 (1.7); 7.4529 (1.8); 7.4359 (2.5); 7.4326
(3.4); 7.4297 (2.2); 7.4125 (2.2); 7.4089 (2.2); 7.2854 (2.2); 7.2763 (2.6); 7.2738 (2.6); 7.2621 (17.4); 7.2557 (1.9); 7.2532 (2.0);
7.2441 (1.6); 6.9214 (2.8); 6.9141 (2.7); 6.9003 (2.7); 6.8929 (2.5); 5.3358 (1.2); 5.3185 (4.6); 5.3011 (4.8); 5.2837 (1.4); 3.7551
(0.5); 1.8557 (0.6); 1.6786 (15.6); 1.6613 (16.0); 1.6488 (0.9); 1.6448 (0.7); 1.5896 (0.7); 1.5719 (1.5); 1.5555 (3.0); 1.5422 (1.0);
1.5382 (1.8); 1.5212 (0.8); 1.4320 (3.3); 0.7763 (1.4); 0.7649 (10.4); 0.7587 (5.7); 0.7530 (6.0); 0.7439 (10.1); 0.7212 (0.5);
0.0079 (0.7); −0.0002 (23.6); −0.0084 (0.6)
I-343: ¹H-NMR(400.6 MHz, CDCl3):
δ = 8.2530 (2.2); 8.2514 (2.5); 8.2494 (2.5); 8.2479 (2.2); 8.2414 (2.2); 8.2398 (2.5); 8.2378 (2.3); 8.1339 (3.5); 8.1277 (3.6);
7.8537 (1.6); 7.8474 (1.6); 7.8349 (1.8); 7.8325 (2.0); 7.8287 (1.8); 7.8262 (1.9); 7.8138 (1.6); 7.8076 (1.6); 7.4991 (1.6); 7.4954
(1.7); 7.4784 (2.3); 7.4749 (2.7); 7.4724 (1.9); 7.4554 (2.2); 7.4517 (2.1); 7.3461 (2.2); 7.3371 (2.6); 7.3345 (2.2); 7.3254 (3.7);
7.3164 (1.7); 7.3138 (1.7); 7.3047 (1.5); 7.2606 (79.4); 6.9661 (2.2); 6.9646 (2.3); 6.9587 (2.2); 6.9573 (2.1); 6.9449 (2.1); 6.9434
(2.2); 6.9375 (2.1); 6.9361 (2.0); 5.3652 (1.2); 5.3477 (4.7); 5.3429 (0.6); 5.3303 (4.8); 5.3129 (1.3); 1.7452 (16.0); 1.7359 (1.4);
1.7278 (15.9); 1.7186 (1.0); 1.4321 (1.7); 1.2643 (1.3); 0.8988 (0.7); 0.8820 (2.4); 0.8643 (0.9); 0.0080 (3.4); −0.0002
(118.0); −0.0085 (2.9)
I-345: ¹H-NMR(400.6 MHz, CDCl3):
δ = 8.2208 (1.1); 8.2187 (0.8); 8.2166 (0.9); 8.2146 (1.1); 8.0913 (0.7); 8.0893 (0.7); 8.0867 (0.8); 8.0846 (0.7); 8.0792 (0.7);
8.0771 (0.8); 8.0746 (0.8); 8.0725 (0.7); 7.8707 (0.6); 7.8646 (0.6); 7.8519 (0.7); 7.8496 (0.7); 7.8457 (0.7); 7.8434 (0.7); 7.8308
(0.6); 7.8246 (0.6); 7.8135 (0.6); 7.8087 (0.6); 7.7951 (0.6); 7.7928 (0.9); 7.7904 (0.7); 7.7881 (0.9); 7.7745 (0.9); 7.7698 (0.9);
7.7114 (0.9); 7.7091 (1.6); 7.7068 (1.0); 7.6908 (0.6); 7.6885 (1.0); 7.6861 (0.6); 7.2615 (14.8); 7.1572 (0.8); 7.1546 (0.8); 7.1451
(0.7); 7.1424 (0.8); 7.1389 (0.8); 7.1363 (0.7); 7.1268 (0.7); 7.1241 (0.7); 7.0140 (0.8); 7.0125 (0.8); 7.0066 (0.8); 7.0051 (0.7);
6.9928 (0.8); 6.9914 (0.7); 6.9854 (0.8); 6.9840 (0.7); 6.9540 (1.2); 6.9419 (1.3); 6.9147 (1.3); 6.9026 (1.4); 5.9312 (1.4); 5.9267
(1.3); 5.8919 (1.2); 5.8874 (1.3); 5.3003 (2.1); 4.9584 (1.0); 4.9201 (3.1); 4.8918 (3.3); 4.8534 (1.1); 3.6910 (16.0); 1.5759 (0.8);
1.3925 (5.0); 1.3749 (4.9); 0.0079 (0.6); −0.0002 (21.3); −0.0085 (0.6)
I-346: ¹H-NMR(400.6 MHz, CDCl3):
δ = 8.1681 (1.4); 8.1645 (1.5); 8.1564 (1.5); 8.1528 (1.5); 8.1271 (0.8); 8.1251 (1.2); 8.1229 (0.9); 8.1209 (0.9); 8.1188 (1.2);
8.1169 (0.8); 7.7841 (0.7); 7.7778 (0.7); 7.7653 (0.8); 7.7629 (0.8); 7.7590 (0.8); 7.7566 (0.8); 7.7440 (0.7); 7.7378 (0.7); 7.3500
(1.3); 7.3383 (1.3); 7.3293 (1.8); 7.3176 (1.8); 7.2638 (6.3); 7.2212 (1.3); 7.2176 (1.4); 7.2003 (1.0); 7.1968 (1.0); 6.9101 (0.8);
6.9085 (0.9); 6.9026 (0.9); 6.9010 (0.8); 6.8888 (0.8); 6.8873 (0.8); 6.8813 (0.8); 6.8798 (0.8); 5.3006 (4.8); 4.9718 (8.7); 3.7922
(16.0); 3.5623 (15.6); 1.5869 (1.3); 0.0700 (0.7); −0.0002 (9.8)
I-347: ¹H-NMR(400.0 MHz, d₆-DMSO):
δ = 8.2626 (3.0); 8.2565 (3.2); 8.1086 (1.7); 8.1068 (2.0); 8.1041 (2.1); 8.1023 (1.9); 8.0965 (1.8); 8.0947 (2.1); 8.0919 (2.1);
8.0901 (1.9); 8.0347 (1.2); 8.0285 (1.1); 8.0138 (1.8); 8.0076 (1.8); 7.9939 (1.3); 7.9873 (2.5); 7.9825 (1.4); 7.9684 (1.9); 7.9666
(2.0); 7.9638 (1.8); 7.9619 (1.8); 7.9479 (1.7); 7.9432 (1.6); 7.6534 (3.6); 7.6328 (3.2); 7.2998 (2.0); 7.2935 (2.0); 7.2864 (2.0);
7.2841 (2.0); 7.2783 (2.1); 7.2719 (3.6); 7.2680 (2.0); 7.2656 (1.8); 7.2558 (1.8); 7.2533 (1.7); 4.8589 (4.9); 4.8471 (5.0); 4.0383
(1.1); 4.0205 (1.1); 3.3261 (3.5); 2.5245 (0.5); 2.5198 (0.7); 2.5111 (14.7); 2.5065 (33.0); 2.5020 (46.5); 2.4974 (33.2); 2.4928
(15.2); 2.3020 (0.8); 2.2901 (0.8); 2.2849 (1.1); 2.2730 (1.1); 2.2678 (0.8); 2.2559 (0.8); 1.9887 (4.9); 1.9096 (2.8); 1.1925 (1.3);
1.1817 (0.8); 1.1747 (2.8); 1.1699 (1.2); 1.1569 (1.3); 1.0879 (9.2); 1.0698 (16.0); 1.0521 (9.7); 1.0387 (0.7); 0.0080 (0.8); −0.0002
(30.2); −0.0085 (1.0)
I-348: ¹H-NMR(400.6 MHz, CDCl3):
δ = 8.1493 (2.0); 8.1431 (2.2); 7.8196 (1.1); 7.8133 (1.0); 7.8006 (1.2); 7.7985 (1.3); 7.7944 (1.3); 7.7923 (1.3); 7.7795 (1.1);
7.7734 (1.1); 7.5626 (1.0); 7.5475 (1.0); 7.5411 (1.4); 7.5261 (1.4); 7.5205 (1.2); 7.5054 (1.1); 7.2624 (13.7); 6.9649 (1.4); 6.9635
(1.5); 6.9575 (1.4); 6.9561 (1.4); 6.9438 (1.4); 6.9424 (1.4); 6.9363 (1.4); 6.9349 (1.4); 6.8676 (1.1); 6.8615 (1.2); 6.8583 (1.2);
6.8521 (1.2); 6.8459 (1.0); 6.8397 (1.1); 6.8365 (1.1); 6.8304 (1.0); 4.8688 (15.9); 4.2810 (1.9); 4.2632 (6.1); 4.2454 (6.2); 4.2276
(2.0); 2.0454 (2.4); 1.5660 (5.0); 1.5552 (0.7); 1.5490 (1.9); 1.5410 (0.6); 1.5346 (0.8); 1.5283 (1.0); 1.5145 (0.6); 1.3092 (7.7);
1.2914 (16.0); 1.2774 (1.2); 1.2736 (7.8); 1.2646 (0.9); 1.2596 (1.9); 1.2418 (0.7); 0.8818 (1.5); 0.8641 (0.6); 0.8025 (1.5); 0.7967
(3.2); 0.7923 (2.5); 0.7896 (1.8); 0.7864 (1.8); 0.7827 (3.9); 0.7783 (5.0); 0.7727 (1.6); 0.7625 (2.1); 0.7589 (1.7); 0.7565 (2.6);
0.7542 (1.6); 0.7511 (1.7); 0.0080 (0.5); −0.0002 (19.2); −0.0085 (0.6)
I-349: ¹H-NMR(400.6 MHz, CDCl3):
δ = 8.2007 (2.5); 8.1946 (2.5); 8.0339 (1.6); 8.0312 (1.7); 8.0236 (1.6); 8.0218 (1.6); 8.0190 (1.6); 7.8576 (1.2); 7.8515 (1.2);
7.8385 (1.5); 7.8366 (1.5); 7.8324 (1.5); 7.8305 (1.4); 7.8176 (1.2); 7.8114 (1.2); 7.7503 (1.1); 7.7456 (1.1); 7.7296 (1.8); 7.7273
(1.4); 7.7250 (1.6); 7.7113 (1.7); 7.7066 (1.6); 7.6429 (3.4); 7.6223 (2.2); 7.3781 (0.6); 7.2603 (40.2); 7.0867 (1.5); 7.0842 (1.6);
7.0746 (1.6); 7.0720 (1.6); 7.0685 (1.6); 7.0659 (1.4); 7.0564 (1.5); 7.0537 (1.4); 6.9901 (1.8); 6.9830 (1.8); 6.9690 (1.7); 6.9619
(1.7); 4.9008 (4.6); 4.8878 (4.6); 4.2952 (1.2); 4.2929 (1.2); 4.2775 (3.8); 4.2751 (3.7); 4.2596 (4.0); 4.2575 (3.7); 4.2418 (1.4);
2.3924 (0.8); 2.3792 (0.9); 2.3752 (1.1); 2.3623 (1.1); 2.3581 (0.9); 2.3451 (0.8); 1.5440 (15.5); 1.3043 (7.9); 1.2865 (16.0);
1.2687 (9.4); 1.1545 (11.0); 1.1465 (11.2); 1.1375 (11.3); 1.1293 (10.7); 0.8989 (1.5); 0.8820 (4.8); 0.8643 (1.9); 0.0080
(1.6); −0.0002 (57.2); −0.0084 (1.8)
I-350: ¹H-NMR(400.6 MHz, d₆-DMSO):
δ = 12.6373 (2.1); 8.5246 (2.4); 8.5055 (2.4); 8.4816 (2.5); 8.4627 (2.5); 8.2625 (5.5); 8.2594 (4.4); 8.2566 (5.6); 8.0989 (2.0);
8.0969 (2.1); 8.0943 (2.5); 8.0922 (2.2); 8.0868 (2.3); 8.0846 (3.1); 8.0819 (4.0); 8.0800 (3.4); 8.0768 (2.2); 8.0713 (2.1); 8.0693
(2.4); 8.0667 (2.4); 8.0646 (2.1); 8.0343 (1.6); 8.0314 (1.7); 8.0281 (1.6); 8.0252 (1.6); 8.0128 (2.8); 8.0101 (2.7); 8.0067 (2.8);
7.9935 (1.9); 7.9906 (3.5); 7.9859 (2.6); 7.9720 (2.2); 7.9699 (2.2); 7.9673 (2.0); 7.9653 (2.1); 7.9554 (1.7); 7.9513 (3.2); 7.9467
(2.2); 7.9371 (1.8); 7.9347 (2.4); 7.9324 (1.8); 7.9300 (2.2); 7.9164 (2.5); 7.9117 (2.3); 7.8528 (2.7); 7.8505 (4.5); 7.8482 (2.6);
7.8322 (1.9); 7.8299 (3.0); 7.8275 (1.6); 7.7205 (2.4); 7.7183 (4.1); 7.7162 (2.3); 7.6999 (2.2); 7.6977 (3.6); 7.6954 (1.9); 7.3064
(3.6); 7.2996 (3.6); 7.2852 (3.7); 7.2804 (4.6); 7.2782 (5.9); 7.2761 (4.1); 7.2733 (2.5); 7.2686 (2.2); 7.2661 (2.5); 7.2621 (3.4);
7.2576 (2.4); 7.2549 (2.0); 7.2501 (2.3); 7.2475 (2.4); 7.2455 (2.3); 7.2428 (2.0); 6.8711 (1.1); 6.6408 (0.7); 5.1941 (1.3); 5.1777
(5.4); 5.1609 (5.4); 5.1439 (1.3); 4.3196 (2.0); 4.3120 (2.1); 4.3011 (3.0); 4.2936 (3.2); 4.2825 (2.1); 4.2751 (2.1); 3.6217 (0.9);
3.6177 (4.8); 3.6155 (2.8); 3.6115 (2.7); 3.6093 (2.1); 3.6075 (3.6); 3.6012 (11.5); 3.5990 (4.1); 3.5950 (3.7); 3.5930 (2.0); 3.5909
(2.5); 3.5867 (2.8); 3.5846 (5.1); 3.5807 (0.9); 3.3223 (35.7); 3.2025 (1.8); 2.6753 (0.7); 2.6706 (1.0); 2.6660 (0.7); 2.5244 (2.8);
2.5197 (3.8); 2.5110 (57.0); 2.5065 (124.1); 2.5019 (172.6); 2.4973 (117.1); 2.4927 (51.0); 2.3336 (0.8); 2.3289 (1.0); 2.3243
(0.7); 2.1834 (1.7); 1.9950 (1.5); 1.7808 (0.8); 1.7761 (5.1); 1.7685 (4.2); 1.7642 (2.4); 1.7595 (14.5); 1.7548 (2.7); 1.7506 (4.0);
1.7429 (4.8); 1.7385 (0.8); 1.5354 (11.8); 1.5309 (11.8); 1.5186 (12.1); 1.5140 (11.4); 1.3555 (15.5); 1.3159 (15.8); 1.3142 (14.9);
1.2977 (16.0); 1.2961 (14.8); 1.2354 (1.0); 1.1822 (0.5); 1.0898 (0.8); 1.0744 (0.7); 0.0080 (1.6); −0.0002 (60.9); −0.0085 (1.6)
I-351: ¹H-NMR(400.6 MHz, d₆-DMSO):
δ = 8.2782 (2.2); 8.2720 (2.3); 8.0650 (1.0); 8.0587 (1.0); 8.0452 (1.4); 8.0439 (1.4); 8.0390 (1.3); 8.0377 (1.4); 8.0242 (1.1);
8.0179 (1.0); 7.8481 (2.0); 7.8285 (3.0); 7.8089 (2.4); 7.4664 (2.6); 7.4475 (1.7); 7.4461 (2.4); 7.3159 (1.4); 7.3148 (1.4); 7.3091
(1.5); 7.2948 (1.4); 7.2934 (1.4); 7.2878 (1.4); 7.2866 (1.3); 7.1358 (2.5); 7.1170 (2.4); 5.7570 (0.8); 4.9308 (11.6); 3.3250 (8.4);
2.5247 (0.9); 2.5201 (1.2); 2.5114 (16.6); 2.5068 (35.7); 2.5022 (49.4); 2.4976 (33.5); 2.4930 (14.4); 2.0742 (16.0); 0.0079
(1.2); −0.0002 (45.9); −0.0085 (1.2)
I-352: ¹H-NMR(400.6 MHz, CDCl3):
δ = 8.2130 (1.9); 8.2110 (1.5); 8.2090 (1.5); 8.2069 (2.0); 8.2049 (1.4); 7.8733 (1.1); 7.8671 (1.1); 7.8543 (1.3); 7.8522 (1.4);
7.8481 (1.3); 7.8461 (1.3); 7.8332 (1.2); 7.8271 (1.2); 7.6321 (1.7); 7.6123 (2.7); 7.5930 (2.2); 7.4267 (2.3); 7.4064 (1.9); 7.2620
(11.4); 6.9950 (1.4); 6.9936 (1.4); 6.9877 (1.4); 6.9862 (1.4); 6.9739 (1.4); 6.9724 (1.4); 6.9665 (1.4); 6.9650 (1.4); 6.9427 (2.1);
6.9239 (2.0); 4.9408 (15.1); 4.3199 (1.9); 4.3021 (6.1); 4.2843 (6.2); 4.2665 (2.0); 2.1460 (16.0); 1.5686 (2.8); 1.3320 (7.8);
1.3142 (16.0); 1.2964 (7.5); −0.0002 (15.8)
I-353: ¹H-NMR(400.6 MHz, CDCl3):
δ = 8.2646 (1.0); 8.2546 (1.0); 7.9858 (1.3); 7.9795 (1.4); 7.6068 (0.7); 7.6006 (0.6); 7.5857 (0.8); 7.5816 (0.8); 7.5796 (0.8);
7.5667 (0.7); 7.5605 (0.7); 7.4886 (0.7); 7.4848 (0.7); 7.4680 (0.9); 7.4649 (1.2); 7.4615 (0.8); 7.4446 (0.8); 7.4409 (0.8); 7.3189
(0.9); 7.3100 (1.1); 7.3073 (1.0); 7.2984 (1.5); 7.2893 (1.0); 7.2868 (0.8); 7.2778 (0.8); 7.2686 (0.6); 7.2604 (62.6); 6.8563 (1.0);
6.8490 (1.0); 6.8352 (0.9); 6.8278 (0.9); 4.9349 (2.7); 3.7686 (16.0); 1.8496 (3.5); 1.8471 (4.5); 1.7495 (6.0); 1.5659 (4.9); 1.5074
(5.0); 1.5038 (5.0); 0.0079 (2.9); −0.0002 (93.2); −0.0085 (2.9); −0.0109 (0.7); −0.0125 (0.5)
I-354: ¹H-NMR(400.6 MHz, CDCl3):
δ = 8.2658 (1.5); 8.2640 (1.6); 8.2542 (1.6); 8.2523 (1.6); 8.0904 (1.9); 8.0845 (1.9); 7.7956 (0.7); 7.7891 (0.7); 7.7748 (1.1);
7.7687 (1.1); 7.7556 (0.7); 7.7493 (0.7); 7.5431 (1.1); 7.5394 (1.1); 7.5224 (1.6); 7.5190 (2.2); 7.5161 (1.2); 7.4991 (1.4); 7.4954
(1.3); 7.3675 (1.3); 7.3586 (1.5); 7.3559 (1.4); 7.3469 (2.3); 7.3379 (1.1); 7.3353 (1.1); 7.3263 (1.0); 7.2605 (67.3); 6.9630 (1.5);
6.9615 (1.5); 6.9555 (1.5); 6.9417 (1.4); 6.9342 (1.4); 4.9243 (16.0); 4.2988 (2.0); 4.2810 (6.0); 4.2631 (6.1); 4.2453 (2.0); 1.5411
(13.4); 1.3094 (7.6); 1.2916 (15.8); 1.2738 (7.5); 0.0080 (3.1); −0.0002 (102.5); −0.0085 (2.9)
I-355: ¹H-NMR(400.0 MHz, CDCl3):
δ = 8.2516 (1.1); 8.2417 (1.0); 8.0988 (1.5); 8.0926 (1.4); 7.7774 (0.6); 7.7712 (0.6); 7.7584 (0.8); 7.7564 (0.8); 7.7522 (0.8);
7.7502 (0.8); 7.7374 (0.7); 7.7312 (0.6); 7.4869 (0.7); 7.4832 (0.6); 7.4662 (0.9); 7.4630 (1.2); 7.4602 (0.7); 7.4430 (0.8); 7.4393
(0.8); 7.3295 (0.8); 7.3204 (0.9); 7.3180 (0.9); 7.3089 (1.4); 7.2998 (0.7); 7.2973 (0.7); 7.2882 (0.6); 7.2613 (11.6); 6.9501 (1.0);
6.9428 (1.0); 6.9289 (1.0); 6.9216 (0.9); 4.9317 (4.5); 4.9257 (4.4); 3.8007 (0.6); 3.7906 (16.0); 2.4352 (0.6); 2.4255 (1.0); 2.4058
(1.1); 2.3960 (0.5); 2.3763 (0.6); 2.0450 (0.6); 1.8715 (0.7); 1.8517 (0.7); 1.8406 (0.5); 1.7604 (0.5); 1.7477 (0.5); 1.5543 (10.0);
0.0079 (0.5); −0.0002 (15.0)
I-356: ¹H-NMR(400.6 MHz, CDCl3):
δ = 8.4355 (0.7); 8.4317 (0.7); 8.4235 (0.7); 8.4198 (0.7); 8.1339 (1.2); 8.1320 (0.9); 8.1278 (1.3); 8.1087 (0.8); 8.1044 (0.8);
8.0888 (0.8); 8.0845 (0.8); 7.7803 (0.6); 7.7741 (0.6); 7.7612 (0.7); 7.7593 (0.7); 7.7551 (0.7); 7.7530 (0.7); 7.7402 (0.6); 7.7340
(0.6); 7.3786 (0.6); 7.3769 (0.6); 7.3666 (0.6); 7.3649 (0.6); 7.3588 (0.6); 7.3573 (0.6); 7.3469 (0.5); 7.3452 (0.5); 7.2621 (10.9);
6.9101 (0.7); 6.9086 (0.8); 6.9026 (0.8); 6.9012 (0.8); 6.8890 (0.7); 6.8875 (0.8); 6.8815 (0.8); 6.8801 (0.7); 5.3004 (1.6); 4.8233
(7.8); 4.4655 (2.0); 4.4495 (4.1); 4.4334 (2.0); 3.7264 (0.5); 3.6965 (16.0); 3.6925 (1.3); 2.6877 (1.9); 2.6716 (3.9); 2.6556 (1.8);
1.5768 (0.6); 1.5632 (2.6); 1.5555 (0.5); 1.5426 (0.6); 0.8176 (1.0); 0.8118 (1.2); 0.8091 (0.9); 0.8053 (1.5); 0.8004 (0.9); 0.7986
(1.2); 0.7918 (1.1); 0.7890 (1.2); 0.7852 (0.9); 0.7823 (1.2); 0.7778 (0.8); 0.7685 (1.2); 0.7621 (1.3); 0.7597 (0.8); 0.7564
(0.9); −0.0002 (14.1)
I-357: ¹H-NMR(400.6 MHz, CDCl3):
δ = 8.2203 (1.3); 8.2183 (1.0); 8.2162 (1.1); 8.2141 (1.3); 7.8746 (0.7); 7.8684 (0.7); 7.8557 (0.8); 7.8535 (0.9); 7.8495 (0.8);
7.8473 (0.9); 7.8346 (0.7); 7.8284 (0.7); 7.6669 (1.0); 7.6471 (1.8); 7.6277 (1.4); 7.4795 (1.5); 7.4593 (1.2); 7.2624 (9.5); 7.0066
(0.9); 7.0051 (1.0); 6.9992 (1.0); 6.9978 (0.9); 6.9855 (0.9); 6.9840 (1.0); 6.9767 (2.3); 6.9582 (1.3); 4.9426 (7.4); 4.1817 (3.4);
4.1683 (3.4); 3.7927 (16.0); 2.1655 (10.1); 1.6102 (0.5); −0.0002 (13.8)
I-359: ¹H-NMR(400.6 MHz, CDCl3):
δ = 8.1482 (2.1); 8.1463 (1.6); 8.1440 (1.7); 8.1419 (2.3); 8.1329 (1.3); 8.1309 (1.5); 8.1283 (1.5); 8.1263 (1.4); 8.1208 (1.4);
8.1189 (1.5); 8.1162 (1.5); 8.1142 (1.4); 7.9567 (1.2); 7.9505 (1.1); 7.9382 (1.3); 7.9355 (1.4); 7.9319 (1.3); 7.9293 (1.3); 7.9170
(1.3); 7.9108 (1.2); 7.8395 (1.2); 7.8347 (1.3); 7.8207 (1.5); 7.8190 (1.7); 7.8160 (1.5); 7.8143 (1.7); 7.8003 (1.7); 7.7956 (1.6);
7.6423 (1.6); 7.6401 (3.0); 7.6379 (1.8); 7.6219 (1.4); 7.6197 (2.5); 7.6174 (1.4); 7.2616 (27.6); 7.2450 (1.6); 7.2425 (1.6); 7.2329
(1.6); 7.2304 (1.5); 7.2264 (1.5); 7.2238 (1.5); 7.2143 (1.5); 7.2118 (1.4); 7.0427 (1.5); 7.0413 (1.6); 7.0354 (1.5); 7.0341 (1.5);
7.0215 (1.4); 7.0201 (1.5); 7.0142 (1.5); 7.0128 (1.5); 5.3005 (5.6); 5.0446 (15.8); 4.3296 (2.1); 4.3117 (6.4); 4.2939 (6.4); 4.2761
(2.1); 1.5553 (12.3); 1.3330 (7.8); 1.3152 (16.0); 1.2974 (7.6); 0.0080 (1.0); −0.0002 (39.5); −0.0085 (1.2)
I-360: ¹H-NMR(400.6 MHz, CDCl3):
δ = 8.2065 (1.3); 8.2045 (1.9); 8.2024 (1.5); 8.2004 (1.5); 8.1983 (2.0); 8.1963 (1.4); 8.0707 (1.2); 8.0686 (1.4); 8.0660 (1.5);
8.0639 (1.4); 8.0586 (1.4); 8.0565 (1.4); 8.0539 (1.5); 8.0518 (1.4); 7.8636 (1.2); 7.8574 (1.2); 7.8447 (1.3); 7.8425 (1.4); 7.8385
(1.3); 7.8363 (1.4); 7.8236 (1.3); 7.8174 (1.2); 7.7683 (1.1); 7.7636 (1.1); 7.7500 (1.3); 7.7477 (1.8); 7.7453 (1.3); 7.7429 (1.7);
7.7293 (1.8); 7.7246 (1.8); 7.6628 (1.7); 7.6604 (3.1); 7.6580 (2.0); 7.6421 (1.2); 7.6398 (2.0); 7.6373 (1.2); 7.2612 (21.7); 7.1197
(1.5); 7.1170 (1.5); 7.1076 (1.5); 7.1049 (1.5); 7.1014 (1.5); 7.0987 (1.4); 7.0893 (1.5); 7.0865 (1.4); 6.9997 (1.4); 6.9981 (1.6);
6.9923 (1.5); 6.9907 (1.5); 6.9785 (1.4); 6.9770 (1.4); 6.9711 (1.4); 6.9696 (1.4); 5.3002 (2.0); 5.0034 (16.0); 4.8356 (9.5); 4.8294
(9.7); 2.4949 (2.7); 2.4887 (6.0); 2.4825 (2.8); 1.5520 (10.2); 0.0079 (0.8); −0.0002 (31.0); −0.0085 (0.9)
I-361: ¹H-NMR(400.6 MHz, CDCl3):
δ = 8.4355 (0.7); 8.4315 (0.7); 8.4233 (0.7); 8.4195 (0.7); 8.1314 (1.1); 8.1295 (0.9); 8.1273 (0.9); 8.1253 (1.2); 8.1076 (0.8);
8.1033 (0.8); 8.0878 (0.8); 8.0835 (0.8); 7.7798 (0.5); 7.7736 (0.5); 7.7607 (0.7); 7.7587 (0.7); 7.7545 (0.7); 7.7526 (0.6); 7.7397
(0.6); 7.7335 (0.6); 7.3777 (0.6); 7.3761 (0.6); 7.3657 (0.6); 7.3641 (0.5); 7.3580 (0.5); 7.3460 (0.5); 7.2617 (14.8); 6.9103 (0.7);
6.9089 (0.8); 6.9029 (0.8); 6.9015 (0.7); 6.8892 (0.7); 6.8878 (0.7); 6.8818 (0.7); 6.8803 (0.7); 5.3004 (0.6); 4.8256 (7.0); 4.3723
(0.7); 4.3547 (0.7); 4.3452 (1.2); 4.3277 (1.3); 4.2768 (1.3); 4.2620 (1.3); 4.2497 (0.8); 4.2349 (0.7); 3.7746 (0.6); 3.7264 (1.1);
3.6867 (16.0); 3.6833 (1.3); 2.8173 (0.5); 2.8144 (0.7); 2.7996 (0.7); 1.5765 (0.5); 1.5631 (1.0); 1.5565 (4.6); 1.5550 (4.2); 1.5498
(0.6); 1.5423 (0.6); 1.1981 (0.7); 1.1919 (6.4); 1.1800 (0.8); 1.1740 (6.3); 0.8217 (1.0); 0.8162 (1.1); 0.8122 (0.8); 0.8091 (1.6);
0.8045 (0.9); 0.8028 (1.1); 0.7958 (1.2); 0.7901 (1.1); 0.7871 (0.8); 0.7830 (1.1); 0.7786 (0.8); 0.7694 (1.2); 0.7628 (1.2); 0.7571
(0.9); 0.0080 (0.5); −0.0002 (20.6); −0.0085 (0.6)
I-362: ¹H-NMR(400.6 MHz, CDCl3):
δ = 8.1109 (0.5); 7.2632 (5.4); 4.9297 (3.6); 4.3575 (0.9); 4.3457 (0.9); 4.3339 (0.9); 3.6240 (1.0); 3.6121 (0.9); 3.6004 (1.0);
3.4902 (16.0); 3.3684 (8.5); −0.0002 (8.2)
I-365: ¹H-NMR(400.6 MHz, CDCl3):
δ = 8.2533 (0.7); 8.2514 (0.8); 8.2496 (0.9); 8.2478 (0.8); 8.2417 (0.8); 8.2398 (0.9); 8.2380 (0.8); 8.2362 (0.8); 8.0995 (1.1);
8.0974 (0.9); 8.0952 (0.9); 8.0932 (1.2); 7.7786 (0.6); 7.7723 (0.5); 7.7598 (0.6); 7.7575 (0.7); 7.7536 (0.6); 7.7513 (0.7); 7.7387
(0.6); 7.7325 (0.6); 7.4901 (0.6); 7.4864 (0.6); 7.4695 (0.8); 7.4659 (1.0); 7.4632 (0.7); 7.4462 (0.8); 7.4425 (0.8); 7.3311 (0.8);
7.3220 (0.9); 7.3195 (0.8); 7.3105 (1.4); 7.3014 (0.6); 7.2988 (0.6); 7.2899 (0.6); 7.2625 (9.1); 6.9522 (0.8); 6.9507 (0.8); 6.9447
(0.8); 6.9432 (0.8); 6.9311 (0.7); 6.9296 (0.8); 6.9236 (0.8); 6.9221 (0.7); 4.9590 (0.5); 4.9188 (3.8); 4.9082 (3.7); 4.8681 (0.5);
4.4838 (1.9); 4.4678 (4.1); 4.4518 (2.0); 3.6936 (16.0); 2.6981 (1.9); 2.6821 (3.9); 2.6662 (1.8); 2.4163 (0.8); 2.3966 (0.9); 2.1719
(0.7); 1.8647 (0.5); 1.8450 (0.6); 1.5652 (3.4); −0.0002 (13.7)
I-366: ¹H-NMR(400.6 MHz, CDCl3):
δ = 8.2458 (0.7); 8.2439 (0.8); 8.2422 (0.8); 8.2403 (0.7); 8.2341 (0.7); 8.2323 (0.8); 8.2305 (0.8); 8.2287 (0.7); 8.1291 (1.1);
8.1229 (1.1); 7.7709 (0.5); 7.7647 (0.5); 7.7519 (0.6); 7.7499 (0.7); 7.7457 (0.6); 7.7436 (0.6); 7.7308 (0.6); 7.7246 (0.6); 7.4551
(0.6); 7.4513 (0.6); 7.4345 (0.8); 7.4310 (1.0); 7.4281 (0.6); 7.4112 (0.7); 7.4074 (0.7); 7.2954 (0.7); 7.2864 (0.8); 7.2838 (0.7);
7.2748 (1.3); 7.2658 (0.7); 7.2617 (11.7); 7.2542 (0.6); 6.9227 (0.7); 6.9213 (0.7); 6.9153 (0.7); 6.9138 (0.7); 6.9016 (0.7); 6.9002
(0.7); 6.8941 (0.7); 6.8927 (0.7); 5.0555 (8.9); 2.0432 (14.6); 1.9634 (16.0); 1.5841 (1.1); 1.5763 (0.5); 1.5692 (0.7); 1.5641 (0.8);
0.8029 (0.7); 0.7978 (1.8); 0.7942 (1.4); 0.7895 (2.0); 0.7852 (2.7); 0.7798 (1.6); 0.7698 (1.1); 0.7655 (1.6); 0.7596 (0.8); −0.0002
(17.2); −0.0085 (0.6)
I-367: ¹H-NMR(400.0 MHz, CDCl3):
δ = 8.2286 (1.1); 8.2170 (1.1); 8.0756 (1.5); 8.0695 (1.5); 7.7522 (0.6); 7.7460 (0.6); 7.7316 (0.9); 7.7265 (0.8); 7.7121 (0.7);
7.7059 (0.6); 7.4714 (0.7); 7.4677 (0.7); 7.4506 (1.0); 7.4477 (1.2); 7.4276 (0.8); 7.4240 (0.8); 7.3058 (0.8); 7.2966 (1.0); 7.2942
(0.9); 7.2851 (1.4); 7.2760 (0.8); 7.2735 (0.8); 7.2627 (6.2); 6.8870 (1.1); 6.8798 (1.0); 6.8659 (1.0); 6.8586 (1.0); 5.1269 (1.4);
5.1228 (2.0); 5.1188 (1.4); 5.0640 (1.9); 5.0617 (1.7); 4.9433 (9.4); 3.8062 (0.5); 3.8003 (0.6); 3.7907 (16.0); 3.7855 (1.1); 1.9273
(7.2); 1.5743 (2.9); −0.0002 (7.9)
I-368: ¹H-NMR(400.6 MHz, CDCl3):
δ = 8.2391 (1.3); 8.2372 (1.5); 8.2355 (1.6); 8.2336 (1.4); 8.2275 (1.4); 8.2256 (1.6); 8.2238 (1.6); 8.2220 (1.4); 8.0989 (2.0);
8.0970 (1.6); 8.0948 (1.6); 8.0927 (2.1); 7.7416 (1.0); 7.7354 (1.0); 7.7227 (1.2); 7.7205 (1.3); 7.7165 (1.2); 7.7143 (1.3); 7.7016
(1.1); 7.6954 (1.1); 7.4736 (1.1); 7.4698 (1.2); 7.4529 (1.4); 7.4494 (1.9); 7.4465 (1.3); 7.4296 (1.4); 7.4259 (1.4); 7.3087 (1.4);
7.2997 (1.5); 7.2971 (1.5); 7.2881 (2.5); 7.2790 (1.1); 7.2764 (1.2); 7.2674 (1.1); 7.2623 (13.4); 6.9384 (1.4); 6.9369 (1.5); 6.9309
(1.4); 6.9294 (1.5); 6.9173 (1.3); 6.9158 (1.4); 6.9098 (1.4); 6.9083 (1.4); 6.3811 (1.8); 6.3522 (2.2); 6.3369 (2.7); 6.3080 (2.5);
6.0266 (2.6); 6.0221 (2.8); 5.9824 (2.1); 5.9779 (2.0); 5.3175 (2.5); 5.3130 (2.3); 5.2886 (2.2); 5.2841 (2.4); 4.9688 (15.7); 4.9370
(0.6); 4.3004 (1.9); 4.2826 (6.0); 4.2648 (6.1); 4.2470 (2.0); 2.0452 (1.6); 1.5684 (4.2); 1.3144 (7.7); 1.3037 (0.5); 1.2966 (16.0);
1.2858 (0.9); 1.2788 (7.7); 1.2679 (0.8); 1.2595 (1.3); 0.8818 (1.2); −0.0002 (18.9); −0.0085 (0.5)
I-369: ¹H-NMR(400.6 MHz, CDCl3):
δ = 8.1533 (1.2); 8.1513 (0.9); 8.1492 (1.0); 8.1471 (1.3); 7.8993 (0.7); 7.8930 (0.6); 7.8807 (0.8); 7.8781 (0.8); 7.8744 (0.8);
7.8719 (0.8); 7.8595 (0.7); 7.8532 (0.7); 7.6172 (0.6); 7.6023 (0.6); 7.5972 (0.7); 7.5955 (0.8); 7.5823 (0.7); 7.5806 (0.8); 7.5754
(0.7); 7.5605 (0.6); 7.2618 (10.8); 7.0080 (0.8); 7.0065 (0.9); 7.0005 (0.9); 6.9990 (0.9); 6.9868 (0.8); 6.9853 (0.9); 6.9793 (0.9);
6.9778 (0.8); 6.9576 (0.6); 6.9513 (0.7); 6.9484 (0.7); 6.9421 (0.7); 6.9357 (0.6); 6.9295 (0.7); 6.9265 (0.7); 6.9202 (0.6); 4.9429
(8.6); 3.8076 (16.0); 1.5541 (5.8); −0.0002 (14.0)
I-370: ¹H-NMR(400.6 MHz, CDCl3):
δ = 8.2046 (1.0); 8.2026 (0.7); 8.2005 (0.8); 8.1985 (1.0); 8.0773 (0.6); 8.0752 (0.7); 8.0726 (0.7); 8.0705 (0.7); 8.0651 (0.7);
8.0631 (0.7); 8.0604 (0.7); 8.0583 (0.6); 7.8632 (0.6); 7.8570 (0.5); 7.8443 (0.6); 7.8421 (0.7); 7.8382 (0.6); 7.8359 (0.6); 7.8232
(0.6); 7.8170 (0.6); 7.7771 (0.6); 7.7723 (0.6); 7.7586 (0.6); 7.7564 (0.8); 7.7539 (0.6); 7.7517 (0.8); 7.7380 (0.8); 7.7332 (0.8);
7.6284 (0.8); 7.6261 (1.4); 7.6238 (0.8); 7.6078 (0.6); 7.6055 (1.1); 7.6031 (0.6); 7.2609 (16.3); 7.1258 (0.7); 7.1233 (0.7); 7.1137
(0.7); 7.1111 (0.7); 7.1075 (0.7); 7.1049 (0.6); 7.0953 (0.7); 7.0927 (0.6); 6.9994 (0.7); 6.9978 (0.7); 6.9920 (0.7); 6.9905 (0.7);
6.9783 (0.6); 6.9767 (0.7); 6.9709 (0.7); 6.9694 (0.6); 6.3498 (0.7); 6.3476 (2.0); 6.3452 (1.9); 6.3430 (0.7); 5.8973 (0.6); 5.8936
(1.5); 5.8911 (1.5); 5.8874 (0.6); 5.0017 (6.9); 4.9708 (2.2); 4.9683 (3.0); 4.9650 (2.1); 3.7700 (16.0); 1.5471 (1.4); 0.0080
(0.7); −0.0002 (25.6); −0.0085 (0.7)
I-371: ¹H-NMR(400.0 MHz, CDCl3):
δ = 8.1603 (1.2); 8.1569 (1.3); 8.1486 (1.3); 8.1452 (1.3); 8.1362 (1.5); 8.1302 (1.5); 7.7898 (0.7); 7.7835 (0.7); 7.7706 (0.8);
7.7687 (0.9); 7.7645 (0.8); 7.7626 (0.8); 7.7496 (0.7); 7.7434 (0.7); 7.3400 (1.0); 7.3283 (1.0); 7.3193 (1.4); 7.3076 (1.3); 7.2629
(6.8); 7.2092 (1.5); 7.2059 (1.5); 7.1885 (1.1); 7.1851 (1.0); 6.9009 (1.0); 6.8937 (1.0); 6.8797 (1.0); 6.8725 (0.9); 5.3003 (3.5);
4.9497 (9.7); 4.2780 (1.3); 4.2601 (3.8); 4.2423 (3.9); 4.2245 (1.3); 3.5596 (16.0); 1.5808 (1.1); 1.2953 (4.5); 1.2775 (8.9); 1.2596
(4.5); −0.0002 (9.5)
I-372: ¹H-NMR(400.6 MHz, CDCl3):
δ = 8.0832 (1.2); 8.0815 (1.9); 8.0794 (1.5); 8.0774 (1.6); 8.0753 (2.0); 8.0734 (1.4); 7.9998 (1.2); 7.9977 (1.3); 7.9951 (1.4);
7.9929 (1.4); 7.9877 (1.3); 7.9855 (1.4); 7.9829 (1.4); 7.9808 (1.3); 7.7158 (1.2); 7.7096 (1.1); 7.6957 (2.3); 7.6907 (2.2); 7.6887
(1.5); 7.6774 (1.6); 7.6752 (2.7); 7.6728 (1.5); 7.6700 (2.4); 7.6568 (2.0); 7.6520 (1.8); 7.5981 (1.7); 7.5957 (3.0); 7.5933 (1.9);
7.5774 (1.2); 7.5751 (1.9); 7.5726 (1.1); 7.2616 (16.9); 7.0109 (1.6); 7.0081 (1.6); 6.9988 (1.4); 6.9960 (1.6); 6.9928 (1.6); 6.9900
(1.4); 6.9806 (1.4); 6.9778 (1.4); 6.9512 (1.4); 6.9496 (1.5); 6.9438 (1.4); 6.9422 (1.5); 6.9302 (1.3); 6.9286 (1.4); 6.9228 (1.4);
6.9212 (1.4); 4.9628 (14.5); 4.3181 (1.9); 4.3002 (6.0); 4.2824 (6.0); 4.2646 (1.9); 4.1701 (0.6); 4.1670 (1.4); 3.2355 (0.6); 3.2149
(1.0); 3.2133 (1.0); 3.1905 (0.6); 2.4972 (1.2); 2.4917 (0.9); 2.4728 (1.5); 2.4669 (1.5); 2.4504 (0.8); 2.4488 (0.9); 2.4445 (1.0);
2.4421 (1.0); 2.4198 (0.5); 2.1074 (0.6); 2.1051 (0.6); 2.0992 (0.7); 2.0948 (0.8); 2.0924 (0.9); 2.0899 (0.9); 2.0867 (1.2); 2.0815
(0.8); 2.0782 (1.0); 2.0725 (1.0); 2.0652 (1.4); 2.0609 (0.8); 2.0589 (0.8); 2.0524 (0.5); 2.0450 (0.6); 1.8942 (0.6); 1.8753 (0.8);
1.8691 (1.0); 1.8596 (0.7); 1.8570 (0.8); 1.8495 (1.6); 1.8439 (1.1); 1.8367 (1.1); 1.8346 (1.1); 1.8316 (1.0); 1.8260 (1.1); 1.8140
(0.7); 1.8119 (0.7); 1.8076 (0.7); 1.3364 (7.7); 1.3186 (16.0); 1.3008 (7.5); 0.0079 (0.8); −0.0002 (24.1); −0.0085 (0.6)
I-373: ¹H-NMR(400.6 MHz, CDCl3):
δ = 8.2290 (1.7); 8.2270 (1.5); 8.2247 (1.8); 8.1559 (1.2); 8.1539 (1.3); 8.1512 (1.4); 8.1491 (1.3); 8.1437 (1.3); 8.1417 (1.4);
8.1390 (1.4); 8.1369 (1.3); 7.8533 (0.7); 7.8470 (0.7); 7.8338 (1.0); 7.8276 (1.0); 7.8133 (0.7); 7.8072 (0.7); 7.7800 (1.1); 7.7752
(1.1); 7.7615 (1.4); 7.7593 (1.6); 7.7568 (1.4); 7.7546 (1.5); 7.7409 (1.6); 7.7361 (1.5); 7.6036 (1.6); 7.6012 (2.8); 7.5990 (1.6);
7.5829 (1.3); 7.5806 (2.2); 7.5783 (1.2); 7.2605 (49.6); 7.1478 (1.4); 7.1453 (1.4); 7.1356 (1.4); 7.1331 (1.4); 7.1294 (1.4); 7.1268
(1.3); 7.1172 (1.4); 7.1146 (1.3); 6.9982 (1.4); 6.9967 (1.7); 6.9908 (1.4); 6.9893 (1.4); 6.9770 (1.3); 6.9755 (1.4); 6.9696 (1.3);
6.9681 (1.3); 5.0196 (16.0); 2.0471 (1.0); 1.4671 (0.7); 1.4322 (0.6); 1.2598 (0.7); 0.0080 (2.1); 0.0056 (0.6); −0.0002
(72.6); −0.0051 (0.7); −0.0085 (2.0)
I-374: ¹H-NMR(400.6 MHz, CDCl3):
δ = 8.2415 (0.7); 8.2398 (0.8); 8.2379 (0.8); 8.2362 (0.7); 8.2299 (0.8); 8.2281 (0.8); 8.2263 (0.8); 8.2246 (0.7); 8.1209 (1.0);
8.1188 (0.8); 8.1166 (0.9); 8.1147 (1.1); 7.8536 (0.6); 7.8473 (0.5); 7.8348 (0.6); 7.8324 (0.7); 7.8286 (0.6); 7.8261 (0.6); 7.8137
(0.6); 7.8074 (0.6); 7.4995 (0.6); 7.4958 (0.6); 7.4788 (0.8); 7.4753 (0.9); 7.4728 (0.6); 7.4558 (0.7); 7.4520 (0.7); 7.3517 (0.7);
7.3427 (0.8); 7.3401 (0.7); 7.3311 (1.2); 7.3220 (0.6); 7.3194 (0.6); 7.3104 (0.5); 7.2633 (18.6); 6.9662 (0.7); 6.9648 (0.7); 6.9589
(0.8); 6.9573 (0.7); 6.9451 (0.7); 6.9436 (0.7); 6.9376 (0.7); 6.9361 (0.7); 5.4494 (1.6); 5.4321 (1.6); 2.0416 (14.2); 1.9316 (16.0);
1.7781 (5.5); 1.7607 (5.4); 1.5652 (7.9); 0.8819 (0.8); 0.0080 (0.9); −0.0002 (28.3); −0.0085 (0.8)
I-375: ¹H-NMR(400.6 MHz, CDCl3):
δ = 8.1760 (1.1); 8.1741 (1.3); 8.1724 (1.3); 8.1643 (1.2); 8.1625 (1.3); 8.1607 (1.3); 8.1343 (1.7); 8.1281 (1.8); 7.7699 (0.9);
7.7637 (0.8); 7.7507 (1.0); 7.7488 (1.1); 7.7446 (1.0); 7.7427 (1.0); 7.7297 (0.9); 7.7236 (0.9); 7.4599 (0.9); 7.4562 (0.9); 7.4394
(1.1); 7.4360 (1.7); 7.4326 (1.0); 7.4158 (1.1); 7.4121 (1.0); 7.2607 (34.7); 7.2502 (1.3); 7.2474 (1.1); 7.2385 (1.9); 7.2296 (0.9);
7.2269 (0.9); 7.2180 (0.8); 6.9105 (1.2); 6.9044 (1.2); 6.8908 (1.1); 6.8894 (1.1); 6.8833 (1.1); 5.2739 (0.6); 5.2565 (2.4); 5.2392
(2.4); 5.2220 (0.7); 4.2354 (1.4); 4.2176 (4.6); 4.1998 (4.8); 4.1820 (1.6); 1.6328 (9.2); 1.6154 (9.2); 1.5858 (0.8); 1.5781 (0.8);
1.5650 (1.3); 1.5569 (0.6); 1.5469 (16.0); 1.5306 (0.5); 1.2671 (5.9); 1.2494 (11.9); 1.2316 (5.6); 0.8397 (0.6); 0.8263 (1.8);
0.8218 (2.0); 0.8137 (2.6); 0.8088 (2.2); 0.8004 (1.2); 0.7822 (0.6); 0.7774 (1.4); 0.7690 (1.9); 0.7648 (1.2); 0.7565 (1.5); 0.7487
(1.7); 0.7433 (1.3); 0.7366 (0.5); 0.7306 (0.6); 0.0080 (1.3); −0.0002 (50.0); −0.0085 (1.6)
I-376: ¹H-NMR(400.6 MHz, d₆-DMSO):
δ = 8.2560 (2.2); 8.2498 (2.3); 8.0476 (1.0); 8.0414 (1.0); 8.0278 (1.4); 8.0266 (1.4); 8.0216 (1.4); 8.0204 (1.4); 8.0069 (1.1);
8.0006 (1.0); 7.8419 (2.0); 7.8223 (3.1); 7.8027 (2.4); 7.4654 (2.6); 7.4451 (2.4); 7.3110 (1.4); 7.3099 (1.4); 7.3042 (1.5); 7.2899
(1.4); 7.2886 (1.4); 7.2829 (1.4); 7.2818 (1.3); 7.1270 (2.5); 7.1082 (2.4); 5.7572 (1.4); 4.9196 (11.1); 3.3259 (1.7); 2.5204 (0.7);
2.5117 (11.2); 2.5071 (24.1); 2.5025 (33.4); 2.4979 (22.7); 2.4934 (9.9); 2.0651 (16.0); 0.0080 (0.8); −0.0002 (28.8); −0.0085 (0.8)
I-377: ¹H-NMR(400.6 MHz, CDCl3):
δ = 8.2379 (1.0); 8.2358 (0.9); 8.2336 (1.1); 8.2318 (1.0); 8.1164 (0.7); 8.1144 (0.8); 8.1118 (0.9); 8.1097 (0.8); 8.1043 (0.8);
8.1022 (0.9); 8.0996 (0.9); 8.0975 (0.8); 7.8394 (0.7); 7.8331 (0.7); 7.8165 (0.7); 7.8119 (0.9); 7.7981 (0.7); 7.7958 (1.0); 7.7935
(0.8); 7.7910 (1.0); 7.7775 (1.1); 7.7728 (1.0); 7.7178 (1.0); 7.7154 (1.9); 7.7130 (1.1); 7.6971 (0.7); 7.6947 (1.2); 7.6923 (0.7);
7.2617 (15.4); 7.1555 (0.8); 7.1529 (0.9); 7.1434 (0.8); 7.1407 (0.9); 7.1373 (0.9); 7.1346 (0.8); 7.1251 (0.8); 7.1224 (0.8); 7.0020
(0.9); 7.0005 (0.9); 6.9945 (0.9); 6.9931 (0.9); 6.9807 (0.8); 6.9793 (0.9); 6.9733 (0.9); 6.9718 (0.9); 4.9338 (7.5); 4.1817 (3.4);
4.1680 (3.4); 3.7876 (16.0); 1.5618 (9.1); 0.0080 (0.6); −0.0002 (21.7); −0.0085 (0.6)
I-380: ¹H-NMR(400.6 MHz, CDCl3):
δ = 8.2492 (1.4); 8.2474 (1.7); 8.2457 (1.6); 8.2439 (1.4); 8.2376 (1.5); 8.2357 (1.7); 8.2340 (1.6); 8.1298 (2.3); 8.1237 (2.3);
7.7706 (1.0); 7.7644 (1.0); 7.7516 (1.3); 7.7496 (1.3); 7.7454 (1.3); 7.7435 (1.2); 7.7306 (1.1); 7.7244 (1.0); 7.4547 (1.1); 7.4510
(1.0); 7.4340 (1.6); 7.4307 (1.9); 7.4277 (1.2); 7.4108 (1.4); 7.4071 (1.3); 7.2969 (1.3); 7.2878 (1.5); 7.2853 (1.4); 7.2763 (2.3);
7.2671 (1.6); 7.2621 (19.2); 7.2556 (1.0); 6.9239 (1.5); 6.9227 (1.4); 6.9166 (1.5); 6.9028 (1.4); 6.9014 (1.4); 6.8954 (1.4); 5.0361
(16.0); 2.5554 (1.2); 2.5372 (3.1); 2.5204 (1.6); 2.5130 (1.3); 2.4940 (2.8); 2.4750 (1.4); 2.0451 (2.0); 1.8188 (1.2); 1.8054 (1.5);
1.7995 (4.5); 1.7910 (3.0); 1.7885 (3.1); 1.7798 (3.9); 1.7731 (1.4); 1.7599 (1.1); 1.6054 (0.7); 1.5996 (0.9); 1.5923 (0.6); 1.5857
(1.9); 1.5778 (0.7); 1.5709 (1.1); 1.5654 (1.8); 1.5514 (0.7); 1.2772 (0.8); 1.2595 (1.6); 1.2416 (0.6); 0.8819 (1.2); 0.8069 (1.6);
0.8018 (3.6); 0.7980 (2.7); 0.7922 (3.8); 0.7883 (6.3); 0.7836 (3.4); 0.7726 (2.2); 0.7681 (3.1); 0.7622 (1.6); 0.0079 (1.0); −0.0002
(27.5); −0.0085 (0.7)
I-382: ¹H-NMR(400.6 MHz, CDCl3):
δ = 8.2268 (1.0); 8.2152 (1.0); 8.1337 (1.4); 8.1276 (1.3); 7.7740 (0.6); 7.7679 (0.6); 7.7548 (0.8); 7.7485 (0.8); 7.7340 (0.6);
7.7278 (0.6); 7.4641 (0.6); 7.4604 (0.6); 7.4434 (0.9); 7.4401 (1.2); 7.4370 (0.7); 7.4201 (0.8); 7.4164 (0.7); 7.2892 (0.8); 7.2803
(0.9); 7.2777 (0.8); 7.2687 (1.4); 7.2615 (13.9); 7.2481 (0.6); 6.9208 (0.9); 6.9134 (0.9); 6.8997 (0.9); 6.8923 (0.9); 4.8907 (8.0);
4.3897 (0.8); 4.3722 (0.8); 4.3626 (1.3); 4.3451 (1.4); 4.2919 (1.4); 4.2771 (1.4); 4.2648 (0.8); 4.2500 (0.8); 3.7857 (0.6); 3.7134
(0.9); 3.6829 (16.0); 2.8432 (0.5); 2.8256 (0.8); 2.8105 (0.8); 1.5868 (0.6); 1.5711 (1.4); 1.5643 (0.9); 1.5576 (0.9); 1.1871 (6.8);
1.1761 (0.7); 1.1692 (6.6); 0.7811 (7.6); 0.7677 (2.4); 0.7623 (2.0); 0.7601 (2.3); 0.0080 (0.6); −0.0002 (19.6); −0.0085 (0.6)
I-383: ¹H-NMR(400.6 MHz, CDCl3):
δ = 8.2260 (1.0); 8.2243 (1.0); 8.2225 (0.9); 8.2143 (1.0); 8.2126 (1.0); 8.1374 (1.3); 8.1313 (1.3); 7.7758 (0.6); 7.7696 (0.6);
7.7568 (0.7); 7.7548 (0.8); 7.7506 (0.7); 7.7487 (0.8); 7.7358 (0.6); 7.7296 (0.6); 7.4663 (0.6); 7.4626 (0.6); 7.4457 (0.8); 7.4423
(1.2); 7.4392 (0.7); 7.4223 (0.8); 7.4186 (0.8); 7.2893 (0.8); 7.2803 (0.8); 7.2777 (0.8); 7.2687 (1.4); 7.2615 (12.6); 7.2481 (0.6);
6.9191 (0.9); 6.9116 (0.9); 6.8979 (0.9); 6.8906 (0.8); 4.8878 (8.6); 4.4797 (2.1); 4.4637 (4.4); 4.4476 (2.2); 3.6919 (16.0); 2.6983
(2.1); 2.6823 (4.3); 2.6663 (2.0); 1.5889 (0.6); 1.5863 (0.6); 1.5707 (1.2); 1.5522 (0.6); 0.7803 (7.4); 0.7724 (1.1); 0.7672 (2.2);
0.7649 (1.9); 0.7617 (1.8); 0.7592 (2.4); 0.7542 (0.9); −0.0002 (18.0); −0.0085 (0.5)
I-384: ¹H-NMR(400.6 MHz, CDCl3):
δ = 8.2542 (1.3); 8.2523 (1.5); 8.2505 (1.6); 8.2487 (1.5); 8.2426 (1.4); 8.2407 (1.6); 8.2389 (1.6); 8.2371 (1.5); 8.0978 (2.0);
8.0916 (2.2); 7.7783 (1.0); 7.7721 (1.0); 7.7595 (1.2); 7.7572 (1.3); 7.7533 (1.2); 7.7510 (1.2); 7.7384 (1.1); 7.7322 (1.0); 7.4861
(1.1); 7.4824 (1.2); 7.4654 (1.5); 7.4619 (1.8); 7.4592 (1.3); 7.4423 (1.4); 7.4385 (1.4); 7.3282 (1.5); 7.3192 (1.6); 7.3166 (1.5);
7.3076 (2.5); 7.2986 (1.2); 7.2959 (1.2); 7.2870 (1.1); 7.2626 (16.3); 6.9510 (1.4); 6.9495 (1.5); 6.9435 (1.5); 6.9420 (1.5); 6.9299
(1.4); 6.9284 (1.4); 6.9224 (1.4); 6.9209 (1.4); 4.9687 (0.6); 4.9572 (1.0); 4.9371 (0.6); 4.9173 (6.8); 4.9048 (6.7); 4.8649 (1.0);
4.2786 (1.8); 4.2607 (5.6); 4.2429 (5.8); 4.2251 (1.9); 4.1308 (1.1); 4.1130 (1.1); 2.4536 (0.7); 2.4339 (0.8); 2.4242 (1.5); 2.4046
(1.7); 2.3948 (0.8); 2.3752 (0.8); 2.0453 (5.3); 1.9064 (0.6); 1.8953 (1.0); 1.8756 (1.0); 1.8645 (0.7); 1.7601 (0.7); 1.7475 (0.6);
1.7407 (0.5); 1.7304 (0.6); 1.5698 (12.8); 1.3003 (7.7); 1.2968 (1.3); 1.2857 (1.3); 1.2825 (16.0); 1.2774 (2.3); 1.2646 (8.5);
1.2595 (3.8); 1.2416 (1.5); 0.8988 (0.6); 0.8819 (2.2); 0.8642 (0.8); 0.0080 (0.5); −0.0002 (20.6); −0.0085 (0.7)
I-385: ¹H-NMR(400.6 MHz, CDCl3):
δ = 8.2728 (1.4); 8.2710 (1.7); 8.2691 (1.7); 8.2674 (1.6); 8.2611 (1.6); 8.2593 (1.8); 8.2575 (1.7); 8.2557 (1.6); 8.1024 (2.3);
8.0962 (2.4); 7.7431 (1.1); 7.7369 (1.1); 7.7243 (1.3); 7.7221 (1.4); 7.7181 (1.3); 7.7159 (1.3); 7.7032 (1.2); 7.6970 (1.2); 7.4782
(1.2); 7.4745 (1.2); 7.4576 (1.7); 7.4540 (2.0); 7.4513 (1.4); 7.4343 (1.6); 7.4306 (1.5); 7.3275 (1.6); 7.3184 (1.8); 7.3158 (1.6);
7.3068 (2.7); 7.2977 (1.2); 7.2951 (1.3); 7.2861 (1.1); 7.2613 (34.4); 6.9459 (1.5); 6.9444 (1.6); 6.9386 (1.6); 6.9371 (1.5); 6.9248
(1.4); 6.9233 (1.6); 6.9175 (1.5); 6.9160 (1.4); 6.3717 (2.0); 6.3429 (2.4); 6.3275 (2.8); 6.2987 (2.6); 5.9815 (2.7); 5.9772 (2.8);
5.9373 (2.3); 5.9330 (2.2); 5.3237 (2.7); 5.3194 (2.4); 5.3003 (1.2); 5.2948 (2.3); 5.2905 (2.5); 5.0367 (16.0); 5.0039 (0.6); 1.2646
(0.9); 0.8989 (0.5); 0.8819 (2.0); 0.8642 (0.7); 0.0080 (1.2); −0.0002 (44.0); −0.0085 (1.2)
I-387: ¹H-NMR(400.6 MHz, CDCl3):
δ = 8.1734 (1.2); 8.1714 (1.8); 8.1693 (1.4); 8.1673 (1.5); 8.1652 (1.9); 8.1633 (1.3); 8.0549 (1.2); 8.0528 (1.2); 8.0501 (1.4);
8.0480 (1.2); 8.0427 (1.2); 8.0406 (1.3); 8.0380 (1.3); 8.0359 (1.2); 7.8172 (1.1); 7.8110 (1.1); 7.7982 (1.2); 7.7961 (1.3); 7.7920
(1.2); 7.7899 (1.3); 7.7771 (1.1); 7.7709 (1.1); 7.7426 (1.1); 7.7378 (1.0); 7.7242 (1.2); 7.7219 (1.6); 7.7195 (1.2); 7.7172 (1.6);
7.7036 (1.6); 7.6988 (1.6); 7.6311 (1.6); 7.6287 (2.8); 7.6263 (1.7); 7.6105 (1.2); 7.6081 (1.9); 7.6057 (1.1); 7.2617 (11.2); 7.0805
(1.3); 7.0779 (1.3); 7.0684 (1.3); 7.0657 (1.4); 7.0622 (1.4); 7.0596 (1.2); 7.0501 (1.3); 7.0474 (1.2); 6.9883 (1.3); 6.9868 (1.4);
6.9810 (1.4); 6.9794 (1.3); 6.9673 (1.3); 6.9657 (1.3); 6.9599 (1.3); 6.9583 (1.3); 5.2996 (0.8); 4.9608 (0.7); 4.9211 (6.7); 4.9126
(6.6); 4.8729 (0.7); 4.3055 (1.8); 4.2876 (5.7); 4.2698 (5.8); 4.2520 (1.9); 2.3625 (0.7); 2.3428 (0.8); 2.3331 (1.5); 2.3134 (1.6);
2.3037 (0.8); 2.2840 (0.8); 1.8820 (0.6); 1.8710 (1.0); 1.8512 (1.0); 1.8402 (0.7); 1.7024 (0.6); 1.6896 (0.6); 1.6726 (0.5); 1.6708
(0.5); 1.3253 (7.7); 1.3075 (16.0); 1.2896 (7.4); −0.0002 (16.3)
I-390: 1H-NMR(400.6 MHz, d6-DMSO):
δ = 8.6148 (3.0); 8.6079 (3.0); 8.2670 (1.7); 8.2629 (3.1); 8.2591 (1.6); 8.2158 (1.2); 8.2128 (1.6); 8.2041 (1.2); 8.2017 (1.6);
7.9529 (0.9); 7.9494 (1.0); 7.9321 (1.1); 7.9281 (1.6); 7.9242 (1.0); 7.9069 (1.0); 7.9034 (1.0); 7.7567 (1.0); 7.7524 (1.1); 7.7499
(1.1); 7.7455 (1.0); 7.7331 (1.0); 7.7287 (1.1); 7.7263 (1.0); 7.7219 (0.9); 7.5501 (1.1); 7.5408 (1.2); 7.5386 (1.2); 7.5292 (1.8);
7.5200 (0.9); 7.5178 (1.0); 7.5085 (0.8); 6.4211 (1.3); 6.3924 (1.6); 6.3772 (1.7); 6.3483 (1.6); 5.9091 (1.6); 5.9037 (1.7); 5.8651
(1.4); 5.8596 (1.4); 5.3176 (1.7); 5.3123 (1.4); 5.2888 (1.4); 5.2834 (1.6); 5.1128 (0.6); 5.0956 (2.4); 5.0782 (2.4); 5.0609 (0.5);
3.3253 (16.0); 2.5414 (0.9); 2.5244 (1.5); 2.5198 (1.9); 2.5110 (16.7); 2.5065 (35.6); 2.5020 (49.2); 2.4974 (34.7); 2.4929 (15.7);
1.5939 (7.0); 1.5765 (7.0); 0.0080 (0.9); −0.0002 (26.9); −0.0084 (0.8)
I-393: 1H-NMR(400.6 MHz, CDCl3):
δ = 8.4405 (1.5); 8.4336 (1.6); 8.2773 (1.7); 8.2285 (0.8); 8.2266 (1.0); 8.2249 (1.1); 8.2231 (1.0); 8.2169 (0.9); 8.2150 (1.1);
8.2133 (1.1); 8.2115 (1.0); 7.4905 (0.7); 7.4868 (0.7); 7.4698 (1.0); 7.4664 (1.2); 7.4634 (0.9); 7.4465 (0.9); 7.4428 (0.9); 7.3791
(0.7); 7.3748 (0.8); 7.3723 (0.8); 7.3679 (0.8); 7.3570 (0.8); 7.3527 (0.9); 7.3501 (0.9); 7.3458 (0.8); 7.3146 (0.9); 7.3056 (1.1);
7.3030 (1.0); 7.2940 (1.6); 7.2850 (0.8); 7.2824 (0.8); 7.2734 (0.7); 7.2609 (32.7); 6.4130 (1.1); 6.3842 (1.3); 6.3689 (1.6); 6.3400
(1.5); 6.0425 (1.6); 6.0381 (1.7); 5.9983 (1.3); 5.9939 (1.3); 5.3497 (1.6); 5.3453 (1.5); 5.3208 (1.4); 5.3164 (1.5); 4.9894 (8.7);
3.8110 (16.0); 3.8033 (0.8); 1.5566 (12.7); 0.0079 (1.5); 0.0054 (0.6); −0.0002 (46.1); −0.0085 (1.7)
I-394: 1H-NMR(400.6 MHz, CDCl3):
δ = 8.4469 (3.3); 8.4400 (3.4); 8.2891 (1.8); 8.2854 (3.3); 8.2817 (1.8); 8.2441 (1.4); 8.2424 (1.6); 8.2406 (1.6); 8.2389 (1.4);
8.2325 (1.5); 8.2307 (1.7); 8.2290 (1.7); 8.2272 (1.4); 7.5013 (1.1); 7.4977 (1.1); 7.4807 (1.5); 7.4772 (1.9); 7.4744 (1.3); 7.4574
(1.4); 7.4537 (1.4); 7.4032 (1.2); 7.3988 (1.3); 7.3963 (1.3); 7.3919 (1.2); 7.3810 (1.2); 7.3766 (1.3); 7.3742 (1.2); 7.3697 (1.1);
7.3330 (1.4); 7.3239 (1.6); 7.3213 (1.4); 7.3123 (2.4); 7.3033 (1.2); 7.3007 (1.2); 7.2917 (1.0); 7.2624 (12.6); 4.9593 (1.1); 4.9194
(7.0); 4.9066 (6.9); 4.8667 (1.1); 4.2802 (1.8); 4.2624 (5.9); 4.2446 (6.0); 4.2267 (2.0); 2.4857 (0.7); 2.4661 (0.9); 2.4564 (1.6);
2.4368 (1.7); 2.4271 (0.9); 2.4075 (0.9); 1.9022 (0.7); 1.8911 (1.0); 1.8823 (0.5); 1.8801 (0.5); 1.8714 (1.1); 1.8602 (0.8); 1.7763
(0.8); 1.7635 (0.7); 1.7567 (0.6); 1.7443 (0.6); 1.5913 (1.6); 1.3010 (7.8); 1.2832 (16.0); 1.2654 (7.5); 0.0079 (0.6); −0.0002
(18.5); −0.0085 (0.6)
I-397: 1H-NMR(400.6 MHz, CDCl3):
δ = 8.4667 (5.0); 8.4598 (5.1); 8.2978 (2.8); 8.2941 (4.8); 8.2904 (2.7); 8.2582 (2.0); 8.2565 (2.3); 8.2546 (2.4); 8.2530 (2.1);
8.2466 (2.1); 8.2448 (2.4); 8.2429 (2.3); 8.2413 (2.1); 7.5018 (1.6); 7.4981 (1.6); 7.4811 (2.2); 7.4777 (2.5); 7.4750 (1.8); 7.4579
(2.1); 7.4543 (2.0); 7.4251 (1.7); 7.4207 (1.8); 7.4183 (1.8); 7.4139 (1.6); 7.4031 (1.7); 7.3988 (1.9); 7.3963 (1.8); 7.3919 (1.6);
7.3455 (2.0); 7.3365 (2.3); 7.3339 (2.1); 7.3248 (3.5); 7.3158 (1.6); 7.3132 (1.7); 7.3042 (1.4); 7.2611 (43.8); 4.9683 (16.0);
2.4912 (1.0); 2.4708 (1.0); 2.4624 (1.8); 2.4422 (2.2); 2.4330 (1.2); 2.4131 (1.1); 1.8267 (0.8); 1.8152 (1.0); 1.8074 (1.1); 1.7950
(1.4); 1.7848 (1.2); 1.7788 (1.5); 1.7653 (1.9); 1.7595 (0.7); 1.7499 (0.7); 1.7457 (0.5); 1.7363 (0.7); 0.0080 (1.7); 0.0049
(0.5); −0.0002 (64.4); −0.0085 (1.8)
I-399: 1H-NMR(400.6 MHz, CDCl3):
δ = 8.4937 (4.4); 8.4869 (4.3); 8.3003 (2.8); 8.2968 (4.4); 7.5783 (1.2); 7.5634 (1.3); 7.5574 (1.8); 7.5426 (1.8); 7.5362 (1.3);
7.5212 (1.2); 7.4714 (1.5); 7.4671 (1.7); 7.4646 (1.7); 7.4603 (1.4); 7.4497 (1.5); 7.4454 (1.8); 7.4430 (1.6); 7.4386 (1.4); 7.2612
(29.7); 6.8970 (1.3); 6.8908 (1.6); 6.8879 (1.6); 6.8816 (1.4); 6.8752 (1.2); 6.8689 (1.4); 6.8661 (1.4); 6.8598 (1.1); 6.3839 (2.0);
6.3550 (2.4); 6.3398 (2.8); 6.3109 (2.8); 6.0221 (2.9); 6.0180 (3.0); 5.9780 (2.4); 5.9738 (2.3); 5.3692 (2.9); 5.3650 (2.5); 5.3403
(2.5); 5.3362 (2.7); 5.3003 (0.8); 5.0092 (16.0); 1.4322 (1.0); 0.0079 (1.3); −0.0002 (43.8); −0.0085 (1.4)
I-391: 1H-NMR(400.6 MHz, d6-DMSO):
δ = 8.6186 (3.0); 8.6117 (3.1); 8.2722 (1.7); 8.2680 (3.1); 8.2641 (1.6); 8.2257 (1.2); 8.2225 (1.6); 8.2203 (1.2); 8.2141 (1.2);
8.2118 (1.6); 8.2087 (1.2); 7.9565 (1.0); 7.9530 (1.0); 7.9357 (1.1); 7.9316 (1.6); 7.9277 (1.0); 7.9105 (1.0); 7.9069 (1.0); 7.7640
(1.0); 7.7596 (1.1); 7.7571 (1.1); 7.7528 (1.0); 7.7404 (1.1); 7.7360 (1.2); 7.7335 (1.0); 7.7291 (0.9); 7.5568 (1.1); 7.5475 (1.3);
7.5452 (1.2); 7.5359 (1.9); 7.5266 (0.9); 7.5244 (1.0); 7.5151 (0.8); 6.4306 (1.3); 6.4018 (1.6); 6.3867 (1.8); 6.3578 (1.7); 5.9068
(1.7); 5.9015 (1.8); 5.8628 (1.5); 5.8574 (1.4); 5.7567 (0.8); 5.3253 (1.8); 5.3199 (1.4); 5.2964 (1.4); 5.2911 (1.7); 4.8740 (9.1);
3.6011 (0.8); 3.3237 (16.0); 2.5411 (1.2); 2.5352 (1.0); 2.5242 (1.9); 2.5196 (2.3); 2.5108 (20.9); 2.5062 (44.8); 2.5017 (62.1);
2.4971 (43.3); 2.4925 (19.2); 1.7595 (1.0); 0.0079 (1.1); −0.0002 (40.8); −0.0060 (0.5); −0.0085 (1.2)

### B. Formulation Examples

a) A dusting product is obtained by mixing 10 parts by weight of a compound of the formula (I) and/or salts thereof and 90 parts by weight of talc as inert substance and comminuting the mixture in an impact mill.
b) A readily water-dispersible, wettable powder is obtained by mixing 25 parts by weight of a compound of the formula (I) and/or salts thereof, 64 parts by weight of kaolin-containing quartz as inert substance, 10 parts by weight of potassium lignosulfonate and 1 part by weight of sodium oleoylmethyltaurate as wetting agent and dispersant and grinding in a pinned-disc mill.
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range e.g. about 255 to more than 277° C.) and grinding to a fineness of below 5 microns in an attrition ball mill.
d) An emulsifiable concentrate is obtained from 15 parts by weight of a compound of the formula (I) and/or salts thereof, 75 parts by weight of cyclohexanone as solvent and 10 parts by weight of oxethylated nonylphenol as emulsifier.
e) Water-dispersible granules are obtained by mixing
75 parts by weight of a compound of the formula (I) and/or salts thereof,
10 parts by weight of calcium lignosulfonate,
5 parts by weight of sodium lauryl sulfate,
3 parts by weight of polyvinyl alcohol and
7 parts by weight of kaolin,
grinding the mixture in a pinned-disk mill, and granulating the powder in a fluidized bed by spray application of water as a granulating liquid.
f) Water-dispersible granules are also obtained by homogenizing and precomminuting, in a colloid mill,
25 parts by weight of a compound of the formula (I) and/or salts thereof,
5 parts by weight of sodium 2,2′-dinaphthylmethane-6,6′-disulfonate,
2 parts by weight of sodium oleoylmethyltaurate,
1 part by weight of polyvinyl alcohol,
17 parts by weight of calcium carbonate and
50 parts by weight of water,
then grinding the mixture in a bead mill and atomizing and drying the resulting suspension in a spray tower by means of a one-phase nozzle.

### C. Biological Examples

A. Pre-Emergence Herbicidal Action and Crop Plant Compatibility

Seeds of monocotyledonous and dicotyledonous weed plants and crop plants are placed in plastic or organic planting pots and covered with soil. The compounds of the invention, formulated in the form of wettable powders (WP) or as emulsion concentrates (EC), are then applied to the surface of the covering soil as aqueous suspension or emulsion with addition of 0.5% additive at a water application rate equivalent to 600 l/ha. After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

Tables A1 to A13 below show the effects of selected compounds of the general formula (I) on various harmful plants and at an application rate corresponding to 320 g/ha or lower, which were obtained by the experimental procedure mentioned above. The appendices “a”, “b” and “c” give differentiation by dosage used with otherwise the same harmful plants tested.

TABLE A1a
Pre-emergence action at 80 g/ha against ABUTH in %

| Example number | Dosage [g/ha] | ABUTH |
|---|---|---|
| I-159 | 80 | 80 |
| I-175 | 80 | 80 |
| I-18 | 80 | 80 |
| I-184 | 80 | 90 |
| I-187 | 80 | 80 |
| I-188 | 80 | 80 |
| I-189 | 80 | 80 |
| I-196 | 80 | 80 |
| I-199 | 80 | 80 |
| I-208 | 80 | 80 |
| I-218 | 80 | 80 |
| I-325 | 80 | 90 |
| I-327 | 80 | 100 |
| I-331 | 80 | 80 |
| I-332 | 80 | 80 |
| I-341 | 80 | 90 |
| I-343 | 80 | 80 |
| I-73 | 80 | 80 |
| I-79 | 80 | 80 |
| I-86 | 80 | 80 |
| III-24 | 80 | 90 |

TABLE A1b
Pre-emergence action at 3200 g/ha against ABUTH in %

| Example number | Dosage [g/ha] | ABUTH |
|---|---|---|
| I-05 | 320 | 80 |
| I-07 | 320 | 90 |
| I-08 | 320 | 80 |
| I-10 | 320 | 80 |
| I-111 | 320 | 80 |
| I-13 | 320 | 90 |
| I-149 | 320 | 80 |
| I-15 | 320 | 90 |
| I-154 | 320 | 90 |
| I-155 | 320 | 90 |
| I-159 | 320 | 80 |
| I-164 | 320 | 80 |
| I-167 | 320 | 90 |
| I-17 | 320 | 90 |
| I-171 | 320 | 90 |
| I-174 | 320 | 90 |
| I-175 | 320 | 90 |
| I-18 | 320 | 90 |
| I-180 | 320 | 80 |
| I-183 | 320 | 80 |
| I-184 | 320 | 90 |
| I-185 | 320 | 80 |
| I-187 | 320 | 90 |
| I-188 | 320 | 90 |
| I-189 | 320 | 90 |
| I-190 | 320 | 80 |
| I-191 | 320 | 90 |
| I-192 | 320 | 80 |
| I-194 | 320 | 80 |
| I-195 | 320 | 80 |
| I-196 | 320 | 90 |
| I-198 | 320 | 90 |
| I-199 | 320 | 90 |
| I-20 | 320 | 90 |
| I-201 | 320 | 80 |
| I-204 | 320 | 80 |
| I-207 | 320 | 80 |
| I-208 | 320 | 90 |
| I-211 | 320 | 80 |
| I-212 | 320 | 80 |
| I-218 | 320 | 90 |
| I-221 | 320 | 80 |
| I-296 | 320 | 80 |
| I-307 | 320 | 80 |
| I-308 | 320 | 100 |
| I-310 | 320 | 80 |
| I-315 | 320 | 80 |
| I-317 | 320 | 90 |
| I-319 | 320 | 80 |
| I-324 | 320 | 80 |
| I-325 | 320 | 90 |
| I-327 | 320 | 100 |
| I-33 | 320 | 80 |
| I-330 | 320 | 100 |
| I-331 | 320 | 90 |
| I-332 | 320 | 80 |
| I-339 | 320 | 90 |
| I-340 | 320 | 80 |
| I-341 | 320 | 90 |
| I-342 | 320 | 90 |
| I-343 | 320 | 100 |
| I-36 | 320 | 80 |
| I-375 | 320 | 100 |
| I-388 | 320 | 90 |
| I-39 | 320 | 90 |
| I-41 | 320 | 90 |
| I-48 | 320 | 80 |
| I-52 | 320 | 80 |
| I-60 | 320 | 80 |
| I-73 | 320 | 90 |
| I-74 | 320 | 90 |
| I-76 | 320 | 80 |
| I-77 | 320 | 80 |
| I-79 | 320 | 90 |
| I-81 | 320 | 90 |
| I-83 | 320 | 80 |
| I-84 | 320 | 80 |
| I-86 | 320 | 100 |
| I-88 | 320 | 80 |
| I-96 | 320 | 80 |
| III-22 | 320 | 100 |
| III-24 | 320 | 100 |

TABLE A2a
Pre-emergence action at 80 g/ha against ALOMY in %

| Example number | Dosage [g/ha] | ALOMY |
|---|---|---|
| I-02 | 80 | 100 |
| I-03 | 80 | 90 |
| I-05 | 80 | 90 |
| I-07 | 80 | 90 |
| I-08 | 80 | 100 |
| I-10 | 80 | 100 |
| I-100 | 80 | 100 |
| I-102 | 80 | 100 |
| I-107 | 80 | 90 |
| I-11 | 80 | 90 |
| I-116 | 80 | 90 |
| I-118 | 80 | 90 |
| I-122 | 80 | 80 |
| I-13 | 80 | 90 |
| I-148 | 80 | 100 |
| I-149 | 80 | 90 |
| I-15 | 80 | 100 |
| I-154 | 80 | 80 |
| I-159 | 80 | 80 |
| I-16 | 80 | 100 |
| I-164 | 80 | 100 |
| I-167 | 80 | 90 |
| I-17 | 80 | 100 |
| I-171 | 80 | 100 |
| I-174 | 80 | 90 |
| I-175 | 80 | 100 |
| I-18 | 80 | 100 |
| I-180 | 80 | 90 |
| I-184 | 80 | 90 |
| I-185 | 80 | 80 |
| I-187 | 80 | 90 |
| I-188 | 80 | 90 |
| I-189 | 80 | 80 |
| I-19 | 80 | 100 |
| I-190 | 80 | 80 |
| I-191 | 80 | 80 |
| I-192 | 80 | 90 |
| I-194 | 80 | 80 |
| I-195 | 80 | 80 |
| I-198 | 80 | 80 |
| I-199 | 80 | 90 |
| I-20 | 80 | 90 |
| I-204 | 80 | 90 |
| I-208 | 80 | 90 |
| I-210 | 80 | 80 |
| I-211 | 80 | 80 |
| I-212 | 80 | 100 |
| I-218 | 80 | 90 |
| I-225 | 80 | 80 |
| I-24 | 80 | 100 |
| I-247 | 80 | 80 |
| I-25 | 80 | 80 |
| I-29 | 80 | 90 |
| I-291 | 80 | 100 |
| I-293 | 80 | 90 |
| I-296 | 80 | 80 |
| I-30 | 80 | 100 |
| I-307 | 80 | 80 |
| I-308 | 80 | 100 |
| I-311 | 80 | 80 |
| I-313 | 80 | 90 |
| I-315 | 80 | 100 |
| I-316 | 80 | 90 |
| I-317 | 80 | 100 |
| I-32 | 80 | 90 |
| I-322 | 80 | 80 |
| I-323 | 80 | 100 |
| I-324 | 80 | 100 |
| I-33 | 80 | 100 |
| I-331 | 80 | 100 |
| I-333 | 80 | 90 |
| I-334 | 80 | 80 |
| I-336 | 80 | 80 |
| I-337 | 80 | 80 |
| I-338 | 80 | 100 |
| I-339 | 80 | 100 |
| I-34 | 80 | 90 |
| I-342 | 80 | 100 |
| I-343 | 80 | 90 |
| I-35 | 80 | 90 |
| I-350 | 80 | 100 |
| I-358 | 80 | 80 |
| I-36 | 80 | 100 |
| I-360 | 80 | 100 |
| I-369 | 80 | 80 |
| I-37 | 80 | 100 |
| I-370 | 80 | 80 |
| I-374 | 80 | 90 |
| I-375 | 80 | 80 |
| I-38 | 80 | 100 |
| I-388 | 80 | 80 |
| I-39 | 80 | 100 |
| I-40 | 80 | 100 |
| I-41 | 80 | 80 |
| I-42 | 80 | 100 |
| I-43 | 80 | 100 |
| I-44 | 80 | 100 |
| I-45 | 80 | 90 |
| I-46 | 80 | 90 |
| I-47 | 80 | 90 |
| I-48 | 80 | 80 |
| I-51 | 80 | 90 |
| I-52 | 80 | 90 |
| I-53 | 80 | 80 |
| I-56 | 80 | 100 |
| I-59 | 80 | 80 |
| I-64 | 80 | 90 |
| I-66 | 80 | 100 |
| I-73 | 80 | 90 |
| I-74 | 80 | 80 |
| I-75 | 80 | 80 |
| I-76 | 80 | 90 |
| I-79 | 80 | 90 |
| I-80 | 80 | 100 |
| I-81 | 80 | 80 |
| I-83 | 80 | 90 |
| I-84 | 80 | 80 |
| I-85 | 80 | 80 |
| I-86 | 80 | 100 |
| I-87 | 80 | 90 |
| I-88 | 80 | 80 |
| I-90 | 80 | 90 |
| I-91 | 80 | 90 |
| I-93 | 80 | 90 |
| I-94 | 80 | 80 |
| I-96 | 80 | 80 |
| I-97 | 80 | 90 |
| I-98 | 80 | 90 |
| I-99 | 80 | 80 |
| III-22 | 80 | 100 |
| III-24 | 80 | 100 |

TABLE A2b
Pre-emergence action at 320 g/ha against ALOMY in %

| Example number | Dosage [g/ha] | ALOMY |
|---|---|---|
| I-01 | 320 | 100 |
| I-02 | 320 | 90 |
| I-03 | 320 | 100 |
| I-04 | 320 | 100 |
| I-05 | 320 | 100 |
| I-07 | 320 | 100 |
| I-08 | 320 | 100 |
| I-10 | 320 | 100 |
| I-100 | 320 | 100 |
| I-101 | 320 | 100 |
| I-102 | 320 | 100 |
| I-103 | 320 | 100 |
| I-107 | 320 | 90 |
| I-11 | 320 | 100 |
| I-111 | 320 | 90 |
| I-114 | 320 | 100 |
| I-116 | 320 | 100 |
| I-118 | 320 | 100 |
| I-119 | 320 | 100 |
| I-120 | 320 | 80 |
| I-121 | 320 | 100 |
| I-122 | 320 | 100 |
| I-125 | 320 | 80 |
| I-126 | 320 | 80 |
| I-13 | 320 | 100 |
| I-148 | 320 | 100 |
| I-149 | 320 | 100 |
| I-15 | 320 | 100 |
| I-151 | 320 | 100 |
| I-152 | 320 | 80 |
| I-153 | 320 | 90 |
| I-154 | 320 | 90 |
| I-155 | 320 | 100 |
| I-157 | 320 | 80 |
| I-159 | 320 | 90 |
| I-16 | 320 | 100 |
| I-163 | 320 | 90 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-183 | 320 | 80 |
| I-184 | 320 | 100 |
| I-185 | 320 | 90 |
| I-186 | 320 | 90 |
| I-187 | 320 | 100 |
| I-188 | 320 | 100 |
| I-189 | 320 | 90 |
| I-19 | 320 | 100 |
| I-190 | 320 | 90 |
| I-191 | 320 | 90 |
| I-192 | 320 | 90 |
| I-193 | 320 | 90 |
| I-194 | 320 | 100 |
| I-195 | 320 | 90 |
| I-196 | 320 | 90 |
| I-198 | 320 | 90 |
| I-199 | 320 | 90 |
| I-20 | 320 | 100 |
| I-202 | 320 | 80 |
| I-204 | 320 | 100 |
| I-206 | 320 | 80 |
| I-207 | 320 | 80 |
| I-208 | 320 | 100 |
| I-210 | 320 | 90 |
| I-211 | 320 | 100 |
| I-212 | 320 | 100 |
| I-216 | 320 | 90 |
| I-218 | 320 | 90 |
| I-221 | 320 | 90 |
| I-223 | 320 | 90 |
| I-225 | 320 | 90 |
| I-23 | 320 | 100 |
| I-231 | 320 | 90 |
| I-237 | 320 | 90 |
| I-238 | 320 | 90 |
| I-24 | 320 | 100 |
| I-240 | 320 | 90 |
| I-241 | 320 | 80 |
| I-242 | 320 | 80 |
| I-244 | 320 | 90 |
| I-245 | 320 | 90 |
| I-247 | 320 | 100 |
| I-25 | 320 | 90 |
| I-26 | 320 | 100 |
| I-27 | 320 | 100 |
| I-272 | 320 | 90 |
| I-276 | 320 | 80 |
| I-28 | 320 | 100 |
| I-29 | 320 | 100 |
| I-291 | 320 | 100 |
| I-293 | 320 | 100 |
| I-296 | 320 | 100 |
| I-297 | 320 | 80 |
| I-298 | 320 | 90 |
| I-30 | 320 | 100 |
| I-302 | 320 | 90 |
| I-305 | 320 | 90 |
| I-306 | 320 | 90 |
| I-307 | 320 | 90 |
| I-308 | 320 | 100 |
| I-309 | 320 | 90 |
| I-310 | 320 | 100 |
| I-311 | 320 | 90 |
| I-313 | 320 | 100 |
| I-315 | 320 | 100 |
| I-316 | 320 | 100 |
| I-317 | 320 | 100 |
| I-319 | 320 | 100 |
| I-32 | 320 | 100 |
| I-320 | 320 | 90 |
| I-321 | 320 | 100 |
| I-322 | 320 | 100 |
| I-323 | 320 | 100 |
| I-324 | 320 | 100 |
| I-325 | 320 | 100 |
| I-327 | 320 | 100 |
| I-33 | 320 | 100 |
| I-330 | 320 | 100 |
| I-331 | 320 | 100 |
| I-332 | 320 | 100 |
| I-333 | 320 | 100 |
| I-334 | 320 | 90 |
| I-335 | 320 | 80 |
| I-336 | 320 | 100 |
| I-337 | 320 | 90 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 90 |
| I-347 | 320 | 80 |
| I-348 | 320 | 80 |
| I-35 | 320 | 100 |
| I-350 | 320 | 100 |
| I-354 | 320 | 80 |
| I-358 | 320 | 100 |
| I-36 | 320 | 100 |
| I-360 | 320 | 100 |
| I-362 | 320 | 100 |
| I-369 | 320 | 100 |
| I-37 | 320 | 100 |
| I-370 | 320 | 100 |
| I-373 | 320 | 90 |
| I-374 | 320 | 100 |
| I-375 | 320 | 90 |
| I-376 | 320 | 80 |
| I-377 | 320 | 80 |
| I-378 | 320 | 80 |
| I-38 | 320 | 100 |
| I-381 | 320 | 90 |
| I-388 | 320 | 100 |
| I-39 | 320 | 100 |
| I-40 | 320 | 100 |
| I-41 | 320 | 100 |
| I-42 | 320 | 100 |
| I-43 | 320 | 100 |
| I-44 | 320 | 100 |
| I-46 | 320 | 100 |
| I-47 | 320 | 90 |
| I-48 | 320 | 100 |
| I-51 | 320 | 100 |
| I-52 | 320 | 100 |
| I-53 | 320 | 100 |
| I-54 | 320 | 100 |
| I-56 | 320 | 100 |
| I-58 | 320 | 90 |
| I-59 | 320 | 100 |
| I-64 | 320 | 100 |
| I-65 | 320 | 80 |
| I-66 | 320 | 100 |
| I-70 | 320 | 90 |
| I-71 | 320 | 90 |
| I-72 | 320 | 100 |
| I-73 | 320 | 100 |
| I-74 | 320 | 100 |
| I-75 | 320 | 90 |
| I-76 | 320 | 100 |
| I-77 | 320 | 90 |
| I-78 | 320 | 90 |
| I-79 | 320 | 100 |
| I-80 | 320 | 100 |
| I-81 | 320 | 100 |
| I-83 | 320 | 100 |
| I-84 | 320 | 100 |
| I-85 | 320 | 90 |
| I-86 | 320 | 100 |
| I-87 | 320 | 100 |
| I-88 | 320 | 100 |
| I-90 | 320 | 100 |
| I-91 | 320 | 100 |
| I-93 | 320 | 100 |
| I-94 | 320 | 100 |
| I-95 | 320 | 80 |
| I-96 | 320 | 100 |
| I-97 | 320 | 100 |
| I-98 | 320 | 100 |
| I-99 | 320 | 100 |
| II-36 | 320 | 100 |
| III-07 | 320 | 80 |
| III-22 | 320 | 100 |
| III-23 | 320 | 90 |
| III-24 | 320 | 100 |

TABLE A3a
Pre-emergence action at 80 g/ha against AMARE in %

| Example number | Dosage [g/ha] | AMARE |
|---|---|---|
| I-01 | 80 | 80 |
| I-02 | 80 | 100 |
| I-03 | 80 | 90 |
| I-04 | 80 | 90 |
| I-05 | 80 | 90 |
| I-07 | 80 | 90 |
| I-08 | 80 | 100 |
| I-10 | 80 | 100 |
| I-100 | 80 | 90 |
| I-102 | 80 | 80 |
| I-103 | 80 | 80 |
| I-111 | 80 | 90 |
| I-116 | 80 | 90 |
| I-118 | 80 | 90 |
| I-121 | 80 | 100 |
| I-122 | 80 | 80 |
| I-13 | 80 | 90 |
| I-148 | 80 | 100 |
| I-149 | 80 | 90 |
| I-15 | 80 | 100 |
| I-151 | 80 | 90 |
| I-154 | 80 | 90 |
| I-155 | 80 | 90 |
| I-159 | 80 | 90 |
| I-16 | 80 | 100 |
| I-164 | 80 | 100 |
| I-167 | 80 | 100 |
| I-17 | 80 | 100 |
| I-171 | 80 | 100 |
| I-174 | 80 | 100 |
| I-175 | 80 | 100 |
| I-18 | 80 | 100 |
| I-180 | 80 | 100 |
| I-183 | 80 | 80 |
| I-184 | 80 | 100 |
| I-185 | 80 | 100 |
| I-187 | 80 | 80 |
| I-188 | 80 | 100 |
| I-189 | 80 | 100 |
| I-19 | 80 | 100 |
| I-190 | 80 | 90 |
| I-191 | 80 | 90 |
| I-192 | 80 | 80 |
| I-193 | 80 | 80 |
| I-194 | 80 | 100 |
| I-195 | 80 | 90 |
| I-196 | 80 | 100 |
| I-198 | 80 | 100 |
| I-199 | 80 | 100 |
| I-20 | 80 | 100 |
| I-204 | 80 | 90 |
| I-207 | 80 | 90 |
| I-208 | 80 | 90 |
| I-210 | 80 | 80 |
| I-211 | 80 | 100 |
| I-212 | 80 | 90 |
| I-216 | 80 | 90 |
| I-217 | 80 | 80 |
| I-218 | 80 | 100 |
| I-221 | 80 | 80 |
| I-23 | 80 | 90 |
| I-235 | 80 | 80 |
| I-239 | 80 | 80 |
| I-24 | 80 | 90 |
| I-243 | 80 | 80 |
| I-247 | 80 | 80 |
| I-25 | 80 | 90 |
| I-27 | 80 | 90 |
| I-28 | 80 | 90 |
| I-29 | 80 | 90 |
| I-291 | 80 | 80 |
| I-296 | 80 | 80 |
| I-30 | 80 | 100 |
| I-305 | 80 | 80 |
| I-307 | 80 | 100 |
| I-308 | 80 | 100 |
| I-310 | 80 | 80 |
| I-311 | 80 | 80 |
| I-313 | 80 | 90 |
| I-315 | 80 | 90 |
| I-316 | 80 | 100 |
| I-317 | 80 | 100 |
| I-32 | 80 | 90 |
| I-320 | 80 | 80 |
| I-321 | 80 | 100 |
| I-323 | 80 | 100 |
| I-324 | 80 | 100 |
| I-325 | 80 | 100 |
| I-33 | 80 | 90 |
| I-331 | 80 | 100 |
| I-332 | 80 | 80 |
| I-333 | 80 | 90 |
| I-334 | 80 | 90 |
| I-336 | 80 | 90 |
| I-337 | 80 | 90 |
| I-338 | 80 | 100 |
| I-339 | 80 | 100 |
| I-34 | 80 | 100 |
| I-340 | 80 | 90 |
| I-341 | 80 | 100 |
| I-342 | 80 | 100 |
| I-343 | 80 | 100 |
| I-35 | 80 | 100 |
| I-350 | 80 | 100 |
| I-354 | 80 | 80 |
| I-358 | 80 | 80 |
| I-360 | 80 | 90 |
| I-369 | 80 | 90 |
| I-37 | 80 | 100 |
| I-370 | 80 | 100 |
| I-374 | 80 | 100 |
| I-375 | 80 | 100 |
| I-377 | 80 | 80 |
| I-378 | 80 | 80 |
| I-38 | 80 | 100 |
| I-388 | 80 | 90 |
| I-39 | 80 | 100 |
| I-40 | 80 | 80 |
| I-41 | 80 | 100 |
| I-42 | 80 | 90 |
| I-43 | 80 | 80 |
| I-44 | 80 | 80 |
| I-45 | 80 | 90 |
| I-46 | 80 | 100 |
| I-47 | 80 | 100 |
| I-48 | 80 | 90 |
| I-51 | 80 | 100 |
| I-52 | 80 | 80 |
| I-53 | 80 | 90 |
| I-56 | 80 | 100 |
| I-59 | 80 | 100 |
| I-64 | 80 | 100 |
| I-66 | 80 | 90 |
| I-69 | 80 | 80 |
| I-73 | 80 | 100 |
| I-74 | 80 | 90 |
| I-75 | 80 | 80 |
| I-76 | 80 | 90 |
| I-77 | 80 | 90 |
| I-79 | 80 | 100 |
| I-80 | 80 | 90 |
| I-81 | 80 | 80 |
| I-83 | 80 | 90 |
| I-84 | 80 | 80 |
| I-85 | 80 | 90 |
| I-86 | 80 | 100 |
| I-87 | 80 | 90 |
| I-88 | 80 | 80 |
| I-90 | 80 | 100 |
| I-91 | 80 | 90 |
| I-93 | 80 | 90 |
| I-94 | 80 | 80 |
| I-95 | 80 | 90 |
| I-96 | 80 | 90 |
| I-97 | 80 | 90 |
| I-98 | 80 | 100 |
| III-22 | 80 | 90 |
| III-24 | 80 | 100 |

TABLE A3b
Pre-emergence action at 320 g/ha against AMARE in %

| Example number | Dosage [g/ha] | AMARE |
|---|---|---|
| I-01 | 320 | 90 |
| I-02 | 320 | 100 |
| I-03 | 320 | 100 |
| I-04 | 320 | 100 |
| I-05 | 320 | 100 |
| I-07 | 320 | 90 |
| I-08 | 320 | 100 |
| I-10 | 320 | 100 |
| I-100 | 320 | 100 |
| I-101 | 320 | 100 |
| I-102 | 320 | 90 |
| I-103 | 320 | 90 |
| I-107 | 320 | 100 |
| I-11 | 320 | 100 |
| I-111 | 320 | 100 |
| I-116 | 320 | 100 |
| I-118 | 320 | 100 |
| I-119 | 320 | 100 |
| I-120 | 320 | 90 |
| I-121 | 320 | 100 |
| I-122 | 320 | 100 |
| I-125 | 320 | 100 |
| I-126 | 320 | 100 |
| I-13 | 320 | 100 |
| I-147 | 320 | 90 |
| I-148 | 320 | 100 |
| I-149 | 320 | 100 |
| I-15 | 320 | 100 |
| I-150 | 320 | 80 |
| I-151 | 320 | 100 |
| I-152 | 320 | 100 |
| I-153 | 320 | 100 |
| I-154 | 320 | 100 |
| I-155 | 320 | 100 |
| I-156 | 320 | 100 |
| I-159 | 320 | 100 |
| I-16 | 320 | 100 |
| I-160 | 320 | 80 |
| I-162 | 320 | 90 |
| I-163 | 320 | 90 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-183 | 320 | 100 |
| I-184 | 320 | 100 |
| I-185 | 320 | 100 |
| I-186 | 320 | 90 |
| I-187 | 320 | 100 |
| I-188 | 320 | 100 |
| I-189 | 320 | 100 |
| I-19 | 320 | 100 |
| I-190 | 320 | 100 |
| I-191 | 320 | 100 |
| I-192 | 320 | 100 |
| I-193 | 320 | 100 |
| I-194 | 320 | 100 |
| I-195 | 320 | 100 |
| I-196 | 320 | 100 |
| I-198 | 320 | 100 |
| I-199 | 320 | 100 |
| I-20 | 320 | 100 |
| I-202 | 320 | 90 |
| I-204 | 320 | 100 |
| I-205 | 320 | 80 |
| I-206 | 320 | 90 |
| I-207 | 320 | 100 |
| I-208 | 320 | 100 |
| I-210 | 320 | 100 |
| I-211 | 320 | 100 |
| I-212 | 320 | 100 |
| I-216 | 320 | 90 |
| I-217 | 320 | 100 |
| I-218 | 320 | 100 |
| I-221 | 320 | 90 |
| I-223 | 320 | 90 |
| I-225 | 320 | 100 |
| I-229 | 320 | 80 |
| I-23 | 320 | 100 |
| I-230 | 320 | 80 |
| I-231 | 320 | 90 |
| I-235 | 320 | 90 |
| I-236 | 320 | 80 |
| I-237 | 320 | 100 |
| I-238 | 320 | 80 |
| I-239 | 320 | 90 |
| I-24 | 320 | 100 |
| I-240 | 320 | 90 |
| I-241 | 320 | 90 |
| I-242 | 320 | 100 |
| I-243 | 320 | 90 |
| I-244 | 320 | 90 |
| I-245 | 320 | 80 |
| I-246 | 320 | 90 |
| I-247 | 320 | 100 |
| I-25 | 320 | 100 |
| I-26 | 320 | 100 |
| I-27 | 320 | 100 |
| I-272 | 320 | 90 |
| I-276 | 320 | 90 |
| I-277 | 320 | 80 |
| I-278 | 320 | 80 |
| I-28 | 320 | 100 |
| I-29 | 320 | 100 |
| I-291 | 320 | 100 |
| I-293 | 320 | 100 |
| I-296 | 320 | 90 |
| I-297 | 320 | 80 |
| I-298 | 320 | 100 |
| I-30 | 320 | 100 |
| I-300 | 320 | 90 |
| I-302 | 320 | 90 |
| I-305 | 320 | 90 |
| I-306 | 320 | 90 |
| I-307 | 320 | 100 |
| I-308 | 320 | 100 |
| I-309 | 320 | 90 |
| I-31 | 320 | 90 |
| I-310 | 320 | 100 |
| I-311 | 320 | 90 |
| I-312 | 320 | 90 |
| I-313 | 320 | 100 |
| I-315 | 320 | 90 |
| I-316 | 320 | 100 |
| I-317 | 320 | 100 |
| I-319 | 320 | 100 |
| I-32 | 320 | 100 |
| I-320 | 320 | 90 |
| I-321 | 320 | 100 |
| I-322 | 320 | 100 |
| I-323 | 320 | 100 |
| I-324 | 320 | 100 |
| I-325 | 320 | 100 |
| I-327 | 320 | 100 |
| I-33 | 320 | 100 |
| I-330 | 320 | 100 |
| I-331 | 320 | 100 |
| I-332 | 320 | 100 |
| I-333 | 320 | 100 |
| I-334 | 320 | 100 |
| I-335 | 320 | 90 |
| I-336 | 320 | 100 |
| I-337 | 320 | 90 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 100 |
| I-348 | 320 | 90 |
| I-35 | 320 | 100 |
| I-350 | 320 | 100 |
| I-351 | 320 | 80 |
| I-352 | 320 | 90 |
| I-354 | 320 | 90 |
| I-358 | 320 | 100 |
| I-359 | 320 | 90 |
| I-36 | 320 | 100 |
| I-360 | 320 | 100 |
| I-362 | 320 | 90 |
| I-369 | 320 | 90 |
| I-37 | 320 | 100 |
| I-370 | 320 | 100 |
| I-373 | 320 | 90 |
| I-374 | 320 | 100 |
| I-375 | 320 | 100 |
| I-376 | 320 | 90 |
| I-377 | 320 | 90 |
| I-378 | 320 | 90 |
| I-38 | 320 | 100 |
| I-381 | 320 | 90 |
| I-388 | 320 | 100 |
| I-39 | 320 | 100 |
| I-40 | 320 | 100 |
| I-41 | 320 | 100 |
| I-42 | 320 | 100 |
| I-43 | 320 | 90 |
| I-44 | 320 | 90 |
| I-46 | 320 | 100 |
| I-47 | 320 | 100 |
| I-48 | 320 | 100 |
| I-51 | 320 | 100 |
| I-52 | 320 | 100 |
| I-53 | 320 | 100 |
| I-54 | 320 | 100 |
| I-56 | 320 | 100 |
| I-59 | 320 | 100 |
| I-60 | 320 | 90 |
| I-64 | 320 | 100 |
| I-65 | 320 | 90 |
| I-66 | 320 | 100 |
| I-69 | 320 | 90 |
| I-70 | 320 | 90 |
| I-72 | 320 | 90 |
| I-73 | 320 | 100 |
| I-74 | 320 | 100 |
| I-75 | 320 | 90 |
| I-76 | 320 | 100 |
| I-77 | 320 | 100 |
| I-79 | 320 | 100 |
| I-80 | 320 | 90 |
| I-81 | 320 | 90 |
| I-83 | 320 | 100 |
| I-84 | 320 | 100 |
| I-85 | 320 | 100 |
| I-86 | 320 | 100 |
| I-87 | 320 | 100 |
| I-88 | 320 | 100 |
| I-90 | 320 | 100 |
| I-91 | 320 | 100 |
| I-93 | 320 | 100 |
| I-94 | 320 | 100 |
| I-95 | 320 | 100 |
| I-96 | 320 | 100 |
| I-97 | 320 | 100 |
| I-98 | 320 | 100 |
| I-99 | 320 | 90 |
| II-36 | 320 | 100 |
| III-02 | 320 | 80 |
| III-08 | 320 | 80 |
| III-09 | 320 | 80 |
| III-17 | 320 | 90 |
| III-19 | 320 | 80 |
| III-22 | 320 | 100 |
| III-23 | 320 | 90 |
| III-24 | 320 | 100 |
| III-25 | 320 | 80 |
| III-28 | 320 | 80 |

TABLE A4a
Pre-emergence action at 80 g/ha against AVEFA in %

| Example number | Dosage [g/ha] | AVEFA |
|---|---|---|
| I-02 | 80 | 90 |
| I-08 | 80 | 80 |
| I-100 | 80 | 80 |
| I-148 | 80 | 80 |
| I-149 | 80 | 80 |
| I-15 | 80 | 80 |
| I-164 | 80 | 90 |
| I-167 | 80 | 90 |
| I-17 | 80 | 80 |
| I-171 | 80 | 90 |
| I-18 | 80 | 90 |
| I-180 | 80 | 90 |
| I-184 | 80 | 80 |
| I-188 | 80 | 80 |
| I-189 | 80 | 90 |
| I-19 | 80 | 80 |
| I-194 | 80 | 80 |
| I-196 | 80 | 80 |
| I-199 | 80 | 80 |
| I-20 | 80 | 90 |
| I-208 | 80 | 80 |
| I-212 | 80 | 80 |
| I-218 | 80 | 80 |
| I-25 | 80 | 90 |
| I-291 | 80 | 80 |
| I-293 | 80 | 80 |
| I-307 | 80 | 90 |
| I-308 | 80 | 100 |
| I-317 | 80 | 90 |
| I-32 | 80 | 90 |
| I-323 | 80 | 90 |
| I-33 | 80 | 80 |
| I-330 | 80 | 80 |
| I-331 | 80 | 100 |
| I-333 | 80 | 80 |
| I-338 | 80 | 90 |
| I-339 | 80 | 100 |
| I-34 | 80 | 80 |
| I-340 | 80 | 80 |
| I-342 | 80 | 90 |
| I-343 | 80 | 80 |
| I-370 | 80 | 90 |
| I-375 | 80 | 90 |
| I-41 | 80 | 90 |
| I-52 | 80 | 90 |
| I-77 | 80 | 80 |
| I-83 | 80 | 80 |
| I-84 | 80 | 80 |
| I-87 | 80 | 80 |
| I-94 | 80 | 80 |
| III-22 | 80 | 80 |
| III-24 | 80 | 100 |

TABLE A4b
Pre-emergence action at 320 g/ha against AVEFA in %

| Example number | Dosage [g/ha] | AVEFA |
|---|---|---|
| I-01 | 320 | 80 |
| I-02 | 320 | 100 |
| I-03 | 320 | 80 |
| I-05 | 320 | 90 |
| I-07 | 320 | 90 |
| I-08 | 320 | 90 |
| I-10 | 320 | 100 |
| I-100 | 320 | 90 |
| I-101 | 320 | 90 |
| I-102 | 320 | 90 |
| I-103 | 320 | 80 |
| I-107 | 320 | 80 |
| I-11 | 320 | 90 |
| I-111 | 320 | 80 |
| I-116 | 320 | 90 |
| I-118 | 320 | 90 |
| I-119 | 320 | 80 |
| I-121 | 320 | 90 |
| I-122 | 320 | 90 |
| I-13 | 320 | 90 |
| I-147 | 320 | 80 |
| I-148 | 320 | 90 |
| I-149 | 320 | 90 |
| I-15 | 320 | 100 |
| I-154 | 320 | 80 |
| I-155 | 320 | 90 |
| I-159 | 320 | 90 |
| I-16 | 320 | 90 |
| I-164 | 320 | 90 |
| I-167 | 320 | 90 |
| I-17 | 320 | 100 |
| I-171 | 320 | 100 |
| I-174 | 320 | 90 |
| I-175 | 320 | 90 |
| I-18 | 320 | 100 |
| I-180 | 320 | 90 |
| I-183 | 320 | 80 |
| I-184 | 320 | 90 |
| I-185 | 320 | 90 |
| I-186 | 320 | 80 |
| I-187 | 320 | 90 |
| I-188 | 320 | 90 |
| I-189 | 320 | 90 |
| I-19 | 320 | 100 |
| I-190 | 320 | 90 |
| I-191 | 320 | 100 |
| I-192 | 320 | 90 |
| I-193 | 320 | 90 |
| I-194 | 320 | 90 |
| I-195 | 320 | 90 |
| I-196 | 320 | 90 |
| I-198 | 320 | 90 |
| I-199 | 320 | 90 |
| I-20 | 320 | 100 |
| I-204 | 320 | 80 |
| I-207 | 320 | 80 |
| I-208 | 320 | 90 |
| I-211 | 320 | 90 |
| I-212 | 320 | 90 |
| I-218 | 320 | 90 |
| I-223 | 320 | 100 |
| I-225 | 320 | 90 |
| I-23 | 320 | 90 |
| I-231 | 320 | 90 |
| I-24 | 320 | 100 |
| I-247 | 320 | 80 |
| I-25 | 320 | 100 |
| I-26 | 320 | 90 |
| I-27 | 320 | 100 |
| I-272 | 320 | 100 |
| I-28 | 320 | 100 |
| I-29 | 320 | 90 |
| I-291 | 320 | 90 |
| I-293 | 320 | 90 |
| I-296 | 320 | 90 |
| I-298 | 320 | 90 |
| I-30 | 320 | 100 |
| I-306 | 320 | 80 |
| I-307 | 320 | 100 |
| I-308 | 320 | 100 |
| I-31 | 320 | 80 |
| I-310 | 320 | 90 |
| I-311 | 320 | 90 |
| I-313 | 320 | 90 |
| I-315 | 320 | 90 |
| I-316 | 320 | 100 |
| I-317 | 320 | 100 |
| I-319 | 320 | 100 |
| I-32 | 320 | 100 |
| I-320 | 320 | 90 |
| I-321 | 320 | 90 |
| I-322 | 320 | 90 |
| I-323 | 320 | 100 |
| I-324 | 320 | 100 |
| I-325 | 320 | 100 |
| I-327 | 320 | 80 |
| I-33 | 320 | 100 |
| I-330 | 320 | 90 |
| I-331 | 320 | 100 |
| I-332 | 320 | 100 |
| I-333 | 320 | 90 |
| I-334 | 320 | 90 |
| I-335 | 320 | 80 |
| I-336 | 320 | 90 |
| I-337 | 320 | 90 |
| I-338 | 320 | 90 |
| I-339 | 320 | 100 |
| I-34 | 320 | 90 |
| I-340 | 320 | 90 |
| I-341 | 320 | 90 |
| I-342 | 320 | 100 |
| I-343 | 320 | 90 |
| I-348 | 320 | 90 |
| I-35 | 320 | 100 |
| I-350 | 320 | 100 |
| I-351 | 320 | 80 |
| I-358 | 320 | 80 |
| I-36 | 320 | 90 |
| I-360 | 320 | 90 |
| I-369 | 320 | 80 |
| I-37 | 320 | 90 |
| I-370 | 320 | 90 |
| I-374 | 320 | 100 |
| I-375 | 320 | 100 |
| I-38 | 320 | 100 |
| I-388 | 320 | 90 |
| I-39 | 320 | 90 |
| I-40 | 320 | 100 |
| I-41 | 320 | 100 |
| I-42 | 320 | 100 |
| I-43 | 320 | 100 |
| I-44 | 320 | 90 |
| I-46 | 320 | 90 |
| I-47 | 320 | 90 |
| I-48 | 320 | 100 |
| I-51 | 320 | 90 |
| I-52 | 320 | 100 |
| I-53 | 320 | 100 |
| I-56 | 320 | 100 |
| I-58 | 320 | 80 |
| I-59 | 320 | 80 |
| I-64 | 320 | 90 |
| I-66 | 320 | 90 |
| I-69 | 320 | 80 |
| I-73 | 320 | 80 |
| I-74 | 320 | 80 |
| I-75 | 320 | 80 |
| I-76 | 320 | 90 |
| I-77 | 320 | 90 |
| I-79 | 320 | 100 |
| I-80 | 320 | 90 |
| I-81 | 320 | 80 |
| I-83 | 320 | 90 |
| I-84 | 320 | 90 |
| I-85 | 320 | 90 |
| I-86 | 320 | 90 |
| I-87 | 320 | 90 |
| I-88 | 320 | 90 |
| I-91 | 320 | 100 |
| I-93 | 320 | 90 |
| I-94 | 320 | 90 |
| I-95 | 320 | 80 |
| I-96 | 320 | 90 |
| I-97 | 320 | 80 |
| I-98 | 320 | 80 |
| I-99 | 320 | 90 |
| II-36 | 320 | 80 |
| III-22 | 320 | 100 |
| III-24 | 320 | 100 |

TABLE A5a
Pre-emergence action at 80 g/ha against DIGSA in %

| Example number | Dosage [g/ha] | DIGSA |
|---|---|---|
| I-01 | 80 | 80 |
| I-03 | 80 | 90 |
| I-05 | 80 | 100 |
| I-07 | 80 | 100 |
| I-08 | 80 | 100 |
| I-10 | 80 | 100 |
| I-11 | 80 | 100 |
| I-118 | 80 | 100 |
| I-119 | 80 | 100 |
| I-120 | 80 | 100 |
| I-121 | 80 | 100 |
| I-122 | 80 | 100 |
| I-126 | 80 | 90 |
| I-13 | 80 | 100 |
| I-148 | 80 | 90 |
| I-149 | 80 | 90 |
| I-15 | 80 | 100 |
| I-154 | 80 | 90 |
| I-159 | 80 | 90 |
| I-16 | 80 | 100 |
| I-163 | 80 | 90 |
| I-164 | 80 | 90 |
| I-167 | 80 | 100 |
| I-17 | 80 | 100 |
| I-171 | 80 | 80 |
| I-174 | 80 | 100 |
| I-175 | 80 | 100 |
| I-18 | 80 | 100 |
| I-180 | 80 | 90 |
| I-19 | 80 | 100 |
| I-192 | 80 | 90 |
| I-194 | 80 | 90 |
| I-195 | 80 | 90 |
| I-208 | 80 | 90 |
| I-212 | 80 | 100 |
| I-216 | 80 | 90 |
| I-221 | 80 | 90 |
| I-231 | 80 | 90 |
| I-247 | 80 | 100 |
| I-29 | 80 | 100 |
| I-291 | 80 | 100 |
| I-293 | 80 | 80 |
| I-296 | 80 | 90 |
| I-298 | 80 | 100 |
| I-30 | 80 | 100 |
| I-300 | 80 | 90 |
| I-302 | 80 | 90 |
| I-322 | 80 | 100 |
| I-348 | 80 | 90 |
| I-349 | 80 | 80 |
| I-36 | 80 | 100 |
| I-360 | 80 | 90 |
| I-37 | 80 | 100 |
| I-38 | 80 | 100 |
| I-39 | 80 | 100 |
| I-45 | 80 | 100 |
| I-46 | 80 | 100 |
| I-54 | 80 | 100 |
| I-56 | 80 | 100 |
| I-59 | 80 | 100 |
| I-64 | 80 | 100 |
| I-66 | 80 | 100 |
| I-90 | 80 | 100 |
| I-97 | 80 | 100 |

TABLE A5b
Pre-emergence action at 320 g/ha against DIGSA in %

| Example number | Dosage [g/ha] | DIGSA |
|---|---|---|
| I-01 | 320 | 100 |
| I-03 | 320 | 100 |
| I-05 | 320 | 100 |
| I-07 | 320 | 100 |
| I-08 | 320 | 100 |
| I-10 | 320 | 100 |
| I-11 | 320 | 100 |
| I-114 | 320 | 90 |
| I-118 | 320 | 100 |
| I-119 | 320 | 100 |
| I-120 | 320 | 100 |
| I-121 | 320 | 100 |
| I-122 | 320 | 100 |
| I-125 | 320 | 100 |
| I-126 | 320 | 100 |
| I-13 | 320 | 100 |
| I-148 | 320 | 100 |
| I-149 | 320 | 100 |
| I-15 | 320 | 100 |
| I-154 | 320 | 100 |
| I-159 | 320 | 100 |
| I-16 | 320 | 100 |
| I-163 | 320 | 100 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-19 | 320 | 100 |
| I-192 | 320 | 100 |
| I-194 | 320 | 100 |
| I-195 | 320 | 100 |
| I-206 | 320 | 90 |
| I-208 | 320 | 100 |
| I-212 | 320 | 100 |
| I-216 | 320 | 100 |
| I-221 | 320 | 100 |
| I-223 | 320 | 80 |
| I-231 | 320 | 100 |
| I-235 | 320 | 90 |
| I-236 | 320 | 90 |
| I-237 | 320 | 90 |
| I-238 | 320 | 90 |
| I-239 | 320 | 90 |
| I-240 | 320 | 90 |
| I-241 | 320 | 90 |
| I-242 | 320 | 90 |
| I-243 | 320 | 80 |
| I-244 | 320 | 90 |
| I-245 | 320 | 90 |
| I-246 | 320 | 90 |
| I-247 | 320 | 100 |
| I-29 | 320 | 100 |
| I-291 | 320 | 100 |
| I-293 | 320 | 100 |
| I-296 | 320 | 100 |
| I-297 | 320 | 80 |
| I-298 | 320 | 100 |
| I-30 | 320 | 100 |
| I-300 | 320 | 100 |
| I-302 | 320 | 100 |
| I-322 | 320 | 100 |
| I-345 | 320 | 90 |
| I-347 | 320 | 90 |
| I-348 | 320 | 100 |
| I-349 | 320 | 90 |
| I-36 | 320 | 100 |
| I-360 | 320 | 100 |
| I-37 | 320 | 100 |
| I-38 | 320 | 100 |
| I-39 | 320 | 100 |
| I-46 | 320 | 100 |
| I-54 | 320 | 100 |
| I-56 | 320 | 100 |
| I-59 | 320 | 100 |
| I-64 | 320 | 100 |
| I-66 | 320 | 100 |
| I-90 | 320 | 100 |
| I-97 | 320 | 100 |
| II-36 | 320 | 100 |

TABLE A6a
Pre-emergence action at 80 g/ha against ECHCG in %

| Example number | Dosage [g/ha] | ECHCG |
|---|---|---|
| I-08 | 80 | 80 |
| I-10 | 80 | 80 |
| I-100 | 80 | 80 |
| I-119 | 80 | 80 |
| I-148 | 80 | 90 |
| I-149 | 80 | 90 |
| I-153 | 80 | 80 |
| I-154 | 80 | 90 |
| I-155 | 80 | 90 |
| I-159 | 80 | 90 |
| I-16 | 80 | 90 |
| I-163 | 80 | 80 |
| I-164 | 80 | 90 |
| I-167 | 80 | 100 |
| I-17 | 80 | 90 |
| I-171 | 80 | 100 |
| I-174 | 80 | 100 |
| I-175 | 80 | 100 |
| I-18 | 80 | 90 |
| I-180 | 80 | 100 |
| I-184 | 80 | 90 |
| I-185 | 80 | 80 |
| I-187 | 80 | 90 |
| I-188 | 80 | 100 |
| I-189 | 80 | 90 |
| I-190 | 80 | 90 |
| I-191 | 80 | 90 |
| I-192 | 80 | 90 |
| I-193 | 80 | 90 |
| I-194 | 80 | 80 |
| I-196 | 80 | 90 |
| I-198 | 80 | 90 |
| I-199 | 80 | 90 |
| I-204 | 80 | 90 |
| I-207 | 80 | 90 |
| I-208 | 80 | 90 |
| I-210 | 80 | 90 |
| I-211 | 80 | 90 |
| I-212 | 80 | 100 |
| I-216 | 80 | 80 |
| I-218 | 80 | 90 |
| I-221 | 80 | 90 |
| I-231 | 80 | 90 |
| I-298 | 80 | 90 |
| I-307 | 80 | 100 |
| I-308 | 80 | 100 |
| I-315 | 80 | 90 |
| I-316 | 80 | 100 |
| I-317 | 80 | 100 |
| I-321 | 80 | 100 |
| I-322 | 80 | 80 |
| I-323 | 80 | 90 |
| I-33 | 80 | 90 |
| I-331 | 80 | 80 |
| I-332 | 80 | 80 |
| I-334 | 80 | 80 |
| I-336 | 80 | 80 |
| I-337 | 80 | 80 |
| I-338 | 80 | 90 |
| I-341 | 80 | 80 |
| I-342 | 80 | 100 |
| I-35 | 80 | 90 |
| I-350 | 80 | 100 |
| I-375 | 80 | 80 |
| I-388 | 80 | 80 |
| I-40 | 80 | 90 |
| I-41 | 80 | 80 |
| I-42 | 80 | 100 |
| I-44 | 80 | 80 |
| I-45 | 80 | 90 |
| I-51 | 80 | 80 |
| I-66 | 80 | 80 |
| I-74 | 80 | 80 |
| I-75 | 80 | 80 |
| I-79 | 80 | 80 |
| I-81 | 80 | 90 |
| I-84 | 80 | 80 |
| I-85 | 80 | 100 |
| I-86 | 80 | 100 |
| I-87 | 80 | 100 |
| I-88 | 80 | 90 |
| I-96 | 80 | 80 |
| III-22 | 80 | 100 |
| III-24 | 80 | 90 |

TABLE A6b
Pre-emergence action at 320 g/ha against ECHCG in %

| Example number | Dosage [g/ha] | ECHCG |
|---|---|---|
| I-01 | 320 | 90 |
| I-02 | 320 | 100 |
| I-03 | 320 | 80 |
| I-04 | 320 | 90 |
| I-05 | 320 | 100 |
| I-07 | 320 | 100 |
| I-08 | 320 | 100 |
| I-10 | 320 | 90 |
| I-100 | 320 | 100 |
| I-101 | 320 | 90 |
| I-107 | 320 | 100 |
| I-11 | 320 | 80 |
| I-111 | 320 | 100 |
| I-116 | 320 | 100 |
| I-118 | 320 | 100 |
| I-119 | 320 | 100 |
| I-121 | 320 | 100 |
| I-122 | 320 | 100 |
| I-125 | 320 | 80 |
| I-126 | 320 | 90 |
| I-13 | 320 | 90 |
| I-148 | 320 | 100 |
| I-149 | 320 | 100 |
| I-15 | 320 | 90 |
| I-151 | 320 | 90 |
| I-152 | 320 | 90 |
| I-153 | 320 | 100 |
| I-154 | 320 | 100 |
| I-155 | 320 | 100 |
| I-156 | 320 | 90 |
| I-157 | 320 | 80 |
| I-159 | 320 | 90 |
| I-16 | 320 | 90 |
| I-160 | 320 | 90 |
| I-162 | 320 | 80 |
| I-163 | 320 | 90 |
| I-164 | 320 | 100 |
| I-167 | 320 | 100 |
| I-17 | 320 | 90 |
| I-171 | 320 | 100 |
| I-174 | 320 | 100 |
| I-175 | 320 | 100 |
| I-18 | 320 | 90 |
| I-180 | 320 | 100 |
| I-183 | 320 | 100 |
| I-184 | 320 | 100 |
| I-185 | 320 | 90 |
| I-186 | 320 | 80 |
| I-187 | 320 | 100 |
| I-188 | 320 | 100 |
| I-189 | 320 | 100 |
| I-19 | 320 | 100 |
| I-190 | 320 | 100 |
| I-191 | 320 | 100 |
| I-192 | 320 | 100 |
| I-193 | 320 | 100 |
| I-194 | 320 | 100 |
| I-195 | 320 | 100 |
| I-196 | 320 | 90 |
| I-198 | 320 | 100 |
| I-199 | 320 | 90 |
| I-20 | 320 | 100 |
| I-201 | 320 | 90 |
| I-202 | 320 | 90 |
| I-204 | 320 | 100 |
| I-205 | 320 | 80 |
| I-206 | 320 | 80 |
| I-207 | 320 | 100 |
| I-208 | 320 | 100 |
| I-210 | 320 | 100 |
| I-211 | 320 | 100 |
| I-212 | 320 | 100 |
| I-216 | 320 | 100 |
| I-217 | 320 | 90 |
| I-218 | 320 | 100 |
| I-221 | 320 | 100 |
| I-223 | 320 | 80 |
| I-225 | 320 | 80 |
| I-229 | 320 | 90 |
| I-23 | 320 | 100 |
| I-230 | 320 | 90 |
| I-231 | 320 | 90 |
| I-24 | 320 | 100 |
| I-247 | 320 | 100 |
| I-25 | 320 | 90 |
| I-27 | 320 | 100 |
| I-276 | 320 | 90 |
| I-277 | 320 | 80 |
| I-28 | 320 | 100 |
| I-29 | 320 | 100 |
| I-291 | 320 | 90 |
| I-293 | 320 | 90 |
| I-296 | 320 | 100 |
| I-298 | 320 | 100 |
| I-30 | 320 | 100 |
| I-300 | 320 | 90 |
| I-302 | 320 | 90 |
| I-305 | 320 | 90 |
| I-307 | 320 | 100 |
| I-308 | 320 | 100 |
| I-309 | 320 | 80 |
| I-31 | 320 | 90 |
| I-311 | 320 | 100 |
| I-313 | 320 | 100 |
| I-315 | 320 | 100 |
| I-316 | 320 | 100 |
| I-317 | 320 | 100 |
| I-319 | 320 | 100 |
| I-32 | 320 | 100 |
| I-320 | 320 | 90 |
| I-321 | 320 | 100 |
| I-322 | 320 | 100 |
| I-323 | 320 | 100 |
| I-324 | 320 | 100 |
| I-33 | 320 | 100 |
| I-330 | 320 | 100 |
| I-331 | 320 | 100 |
| I-332 | 320 | 100 |
| I-333 | 320 | 90 |
| I-334 | 320 | 90 |
| I-336 | 320 | 100 |
| I-337 | 320 | 90 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 100 |
| I-348 | 320 | 90 |
| I-35 | 320 | 100 |
| I-350 | 320 | 100 |
| I-351 | 320 | 90 |
| I-352 | 320 | 90 |
| I-354 | 320 | 100 |
| I-358 | 320 | 100 |
| I-359 | 320 | 80 |
| I-36 | 320 | 100 |
| I-360 | 320 | 100 |
| I-369 | 320 | 100 |
| I-37 | 320 | 100 |
| I-370 | 320 | 100 |
| I-373 | 320 | 90 |
| I-374 | 320 | 100 |
| I-375 | 320 | 100 |
| I-376 | 320 | 90 |
| I-38 | 320 | 100 |
| I-388 | 320 | 100 |
| I-39 | 320 | 100 |
| I-40 | 320 | 100 |
| I-41 | 320 | 100 |
| I-42 | 320 | 100 |
| I-43 | 320 | 100 |
| I-44 | 320 | 100 |
| I-46 | 320 | 90 |
| I-47 | 320 | 90 |
| I-48 | 320 | 100 |
| I-51 | 320 | 90 |
| I-52 | 320 | 100 |
| I-53 | 320 | 80 |
| I-54 | 320 | 100 |
| I-56 | 320 | 100 |
| I-59 | 320 | 100 |
| I-64 | 320 | 100 |
| I-65 | 320 | 90 |
| I-66 | 320 | 100 |
| I-69 | 320 | 90 |
| I-73 | 320 | 90 |
| I-74 | 320 | 100 |
| I-75 | 320 | 100 |
| I-76 | 320 | 90 |
| I-77 | 320 | 100 |
| I-79 | 320 | 100 |
| I-80 | 320 | 100 |
| I-81 | 320 | 100 |
| I-82 | 320 | 90 |
| I-83 | 320 | 100 |
| I-84 | 320 | 100 |
| I-85 | 320 | 100 |
| I-86 | 320 | 100 |
| I-87 | 320 | 100 |
| I-88 | 320 | 100 |
| I-90 | 320 | 100 |
| I-91 | 320 | 100 |
| I-93 | 320 | 100 |
| I-94 | 320 | 80 |
| I-96 | 320 | 100 |
| I-98 | 320 | 100 |
| III-22 | 320 | 100 |
| III-24 | 320 | 100 |

TABLE A7a
Pre-emergence action at 80 g/ha against LOLRI in % [g/ha]

| Example number | Dosage [g/ha] | LOLRI |
|---|---|---|
| I-02 | 80 | 90 |
| I-05 | 80 | 80 |
| I-08 | 80 | 80 |
| I-10 | 80 | 100 |
| I-102 | 80 | 80 |
| I-107 | 80 | 80 |
| I-118 | 80 | 90 |
| I-148 | 80 | 90 |
| I-149 | 80 | 100 |
| I-15 | 80 | 90 |
| I-154 | 80 | 90 |
| I-155 | 80 | 90 |
| I-159 | 80 | 90 |
| I-16 | 80 | 90 |
| I-164 | 80 | 100 |
| I-167 | 80 | 100 |
| I-171 | 80 | 100 |
| I-174 | 80 | 100 |
| I-175 | 80 | 100 |
| I-18 | 80 | 90 |
| I-180 | 80 | 100 |
| I-184 | 80 | 100 |
| I-185 | 80 | 100 |
| I-187 | 80 | 90 |
| I-188 | 80 | 100 |
| I-189 | 80 | 100 |
| I-190 | 80 | 100 |
| I-191 | 80 | 90 |
| I-192 | 80 | 80 |
| I-194 | 80 | 90 |
| I-195 | 80 | 80 |
| I-196 | 80 | 100 |
| I-198 | 80 | 100 |
| I-199 | 80 | 100 |
| I-20 | 80 | 100 |
| I-208 | 80 | 90 |
| I-212 | 80 | 80 |
| I-218 | 80 | 100 |
| I-221 | 80 | 80 |
| I-27 | 80 | 100 |
| I-291 | 80 | 90 |
| I-293 | 80 | 100 |
| I-298 | 80 | 90 |
| I-30 | 80 | 90 |
| I-300 | 80 | 80 |
| I-302 | 80 | 80 |
| I-307 | 80 | 100 |
| I-308 | 80 | 100 |
| I-311 | 80 | 90 |
| I-316 | 80 | 100 |
| I-317 | 80 | 100 |
| I-319 | 80 | 90 |
| I-32 | 80 | 90 |
| I-321 | 80 | 100 |
| I-322 | 80 | 90 |
| I-323 | 80 | 100 |
| I-324 | 80 | 100 |
| I-33 | 80 | 90 |
| I-331 | 80 | 90 |
| I-332 | 80 | 90 |
| I-333 | 80 | 80 |
| I-336 | 80 | 80 |
| I-338 | 80 | 100 |
| I-339 | 80 | 100 |
| I-34 | 80 | 90 |
| I-340 | 80 | 100 |
| I-341 | 80 | 100 |
| I-342 | 80 | 100 |
| I-343 | 80 | 90 |
| I-348 | 80 | 90 |
| I-35 | 80 | 90 |
| I-350 | 80 | 100 |
| I-358 | 80 | 100 |
| I-37 | 80 | 80 |
| I-370 | 80 | 90 |
| I-374 | 80 | 80 |
| I-375 | 80 | 100 |
| I-388 | 80 | 80 |
| I-40 | 80 | 90 |
| I-41 | 80 | 100 |
| I-43 | 80 | 90 |
| I-44 | 80 | 80 |
| I-46 | 80 | 90 |
| I-48 | 80 | 100 |
| I-52 | 80 | 90 |
| I-56 | 80 | 100 |
| I-59 | 80 | 80 |
| I-64 | 80 | 80 |
| I-66 | 80 | 80 |
| I-73 | 80 | 100 |
| I-75 | 80 | 80 |
| I-76 | 80 | 90 |
| I-77 | 80 | 90 |
| I-79 | 80 | 100 |
| I-81 | 80 | 90 |
| I-83 | 80 | 100 |
| I-84 | 80 | 90 |
| I-86 | 80 | 100 |
| I-91 | 80 | 100 |
| I-94 | 80 | 100 |
| I-96 | 80 | 90 |
| I-97 | 80 | 80 |
| III-22 | 80 | 100 |
| III-24 | 80 | 100 |

TABLE A7b
Pre-emergence action at 320 g/ha against LOLRI in %

| Example number | Dosage [g/ha] | LOLRI |
|---|---|---|
| I-01 | 320 | 100 |
| I-02 | 320 | 100 |
| I-03 | 320 | 100 |
| I-04 | 320 | 90 |
| I-05 | 320 | 100 |
| I-07 | 320 | 100 |
| I-08 | 320 | 100 |
| I-10 | 320 | 100 |
| I-100 | 320 | 100 |
| I-101 | 320 | 100 |
| I-102 | 320 | 100 |
| I-103 | 320 | 100 |
| I-107 | 320 | 100 |
| I-11 | 320 | 100 |
| I-111 | 320 | 100 |
| I-116 | 320 | 100 |
| I-118 | 320 | 100 |
| I-119 | 320 | 100 |
| I-120 | 320 | 80 |
| I-121 | 320 | 100 |
| I-122 | 320 | 100 |
| I-125 | 320 | 80 |
| I-126 | 320 | 100 |
| I-13 | 320 | 90 |
| I-148 | 320 | 100 |
| I-149 | 320 | 100 |
| I-15 | 320 | 100 |
| I-151 | 320 | 90 |
| I-153 | 320 | 80 |
| I-154 | 320 | 100 |
| I-155 | 320 | 100 |
| I-156 | 320 | 80 |
| I-159 | 320 | 100 |
| I-16 | 320 | 100 |
| I-163 | 320 | 90 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-183 | 320 | 90 |
| I-184 | 320 | 100 |
| I-185 | 320 | 100 |
| I-187 | 320 | 100 |
| I-188 | 320 | 100 |
| I-189 | 320 | 100 |
| I-19 | 320 | 100 |
| I-190 | 320 | 100 |
| I-191 | 320 | 90 |
| I-192 | 320 | 100 |
| I-193 | 320 | 90 |
| I-194 | 320 | 100 |
| I-195 | 320 | 100 |
| I-196 | 320 | 100 |
| I-198 | 320 | 100 |
| I-199 | 320 | 100 |
| I-20 | 320 | 100 |
| I-208 | 320 | 100 |
| I-210 | 320 | 80 |
| I-211 | 320 | 80 |
| I-212 | 320 | 90 |
| I-216 | 320 | 80 |
| I-217 | 320 | 100 |
| I-218 | 320 | 100 |
| I-221 | 320 | 100 |
| I-229 | 320 | 80 |
| I-23 | 320 | 100 |
| I-231 | 320 | 80 |
| I-236 | 320 | 90 |
| I-237 | 320 | 90 |
| I-238 | 320 | 90 |
| I-24 | 320 | 100 |
| I-240 | 320 | 90 |
| I-241 | 320 | 90 |
| I-242 | 320 | 90 |
| I-244 | 320 | 100 |
| I-245 | 320 | 90 |
| I-247 | 320 | 100 |
| I-25 | 320 | 100 |
| I-27 | 320 | 100 |
| I-272 | 320 | 80 |
| I-276 | 320 | 100 |
| I-277 | 320 | 90 |
| I-278 | 320 | 80 |
| I-279 | 320 | 80 |
| I-28 | 320 | 100 |
| I-29 | 320 | 100 |
| I-291 | 320 | 100 |
| I-293 | 320 | 100 |
| I-296 | 320 | 100 |
| I-298 | 320 | 100 |
| I-30 | 320 | 100 |
| I-300 | 320 | 90 |
| I-302 | 320 | 90 |
| I-305 | 320 | 90 |
| I-306 | 320 | 100 |
| I-307 | 320 | 100 |
| I-308 | 320 | 100 |
| I-310 | 320 | 100 |
| I-311 | 320 | 90 |
| I-313 | 320 | 100 |
| I-315 | 320 | 100 |
| I-316 | 320 | 100 |
| I-317 | 320 | 100 |
| I-319 | 320 | 100 |
| I-32 | 320 | 100 |
| I-320 | 320 | 100 |
| I-321 | 320 | 100 |
| I-322 | 320 | 100 |
| I-323 | 320 | 100 |
| I-324 | 320 | 100 |
| I-325 | 320 | 100 |
| I-327 | 320 | 100 |
| I-33 | 320 | 100 |
| I-330 | 320 | 90 |
| I-331 | 320 | 100 |
| I-332 | 320 | 100 |
| I-333 | 320 | 100 |
| I-334 | 320 | 90 |
| I-335 | 320 | 90 |
| I-336 | 320 | 100 |
| I-337 | 320 | 100 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 90 |
| I-348 | 320 | 100 |
| I-35 | 320 | 100 |
| I-350 | 320 | 100 |
| I-352 | 320 | 80 |
| I-358 | 320 | 100 |
| I-359 | 320 | 80 |
| I-36 | 320 | 100 |
| I-360 | 320 | 100 |
| I-362 | 320 | 100 |
| I-369 | 320 | 90 |
| I-37 | 320 | 100 |
| I-370 | 320 | 100 |
| I-373 | 320 | 80 |
| I-374 | 320 | 100 |
| I-375 | 320 | 100 |
| I-376 | 320 | 90 |
| I-378 | 320 | 90 |
| I-38 | 320 | 100 |
| I-381 | 320 | 90 |
| I-388 | 320 | 100 |
| I-39 | 320 | 100 |
| I-40 | 320 | 100 |
| I-41 | 320 | 100 |
| I-42 | 320 | 100 |
| I-43 | 320 | 100 |
| I-44 | 320 | 100 |
| I-46 | 320 | 100 |
| I-47 | 320 | 100 |
| I-48 | 320 | 100 |
| I-51 | 320 | 90 |
| I-52 | 320 | 100 |
| I-53 | 320 | 100 |
| I-56 | 320 | 100 |
| I-58 | 320 | 80 |
| I-59 | 320 | 100 |
| I-60 | 320 | 80 |
| I-64 | 320 | 100 |
| I-65 | 320 | 90 |
| I-66 | 320 | 100 |
| I-69 | 320 | 80 |
| I-70 | 320 | 90 |
| I-71 | 320 | 90 |
| I-72 | 320 | 100 |
| I-73 | 320 | 100 |
| I-74 | 320 | 100 |
| I-75 | 320 | 100 |
| I-76 | 320 | 100 |
| I-77 | 320 | 100 |
| I-79 | 320 | 100 |
| I-80 | 320 | 100 |
| I-81 | 320 | 100 |
| I-83 | 320 | 100 |
| I-84 | 320 | 100 |
| I-85 | 320 | 100 |
| I-86 | 320 | 100 |
| I-87 | 320 | 100 |
| I-88 | 320 | 100 |
| I-90 | 320 | 100 |
| I-91 | 320 | 100 |
| I-93 | 320 | 100 |
| I-94 | 320 | 100 |
| I-96 | 320 | 100 |
| I-97 | 320 | 100 |
| I-98 | 320 | 100 |
| I-99 | 320 | 100 |
| II-36 | 320 | 80 |
| III-22 | 320 | 100 |
| III-23 | 320 | 100 |
| III-24 | 320 | 100 |

TABLE A8a
Pre-emergence action at 80 g/ha against MATIN in %

| Example number | Dosage [g/ha] | MATIN |
|---|---|---|
| I-07 | 80 | 80 |
| I-10 | 80 | 80 |
| I-100 | 80 | 80 |
| I-13 | 80 | 80 |
| I-148 | 80 | 90 |
| I-149 | 80 | 90 |
| I-15 | 80 | 100 |
| I-151 | 80 | 90 |
| I-154 | 80 | 90 |
| I-155 | 80 | 90 |
| I-159 | 80 | 90 |
| I-16 | 80 | 100 |
| I-163 | 80 | 90 |
| I-164 | 80 | 90 |
| I-167 | 80 | 90 |
| I-17 | 80 | 90 |
| I-171 | 80 | 90 |
| I-174 | 80 | 90 |
| I-175 | 80 | 90 |
| I-18 | 80 | 100 |
| I-180 | 80 | 90 |
| I-183 | 80 | 90 |
| I-184 | 80 | 90 |
| I-185 | 80 | 80 |
| I-187 | 80 | 90 |
| I-188 | 80 | 90 |
| I-189 | 80 | 90 |
| I-190 | 80 | 90 |
| I-191 | 80 | 90 |
| I-192 | 80 | 90 |
| I-193 | 80 | 90 |
| I-194 | 80 | 90 |
| I-195 | 80 | 90 |
| I-196 | 80 | 90 |
| I-198 | 80 | 90 |
| I-199 | 80 | 90 |
| I-207 | 80 | 90 |
| I-208 | 80 | 90 |
| I-210 | 80 | 90 |
| I-211 | 80 | 90 |
| I-212 | 80 | 80 |
| I-216 | 80 | 90 |
| I-217 | 80 | 80 |
| I-218 | 80 | 90 |
| I-221 | 80 | 90 |
| I-231 | 80 | 90 |
| I-296 | 80 | 80 |
| I-305 | 80 | 80 |
| I-306 | 80 | 90 |
| I-307 | 80 | 90 |
| I-308 | 80 | 100 |
| I-311 | 80 | 80 |
| I-315 | 80 | 80 |
| I-317 | 80 | 100 |
| I-325 | 80 | 100 |
| I-327 | 80 | 80 |
| I-330 | 80 | 80 |
| I-331 | 80 | 100 |
| I-333 | 80 | 80 |
| I-334 | 80 | 80 |
| I-336 | 80 | 80 |
| I-341 | 80 | 90 |
| I-342 | 80 | 100 |
| I-369 | 80 | 80 |
| I-375 | 80 | 100 |
| I-377 | 80 | 80 |
| I-38 | 80 | 100 |
| I-388 | 80 | 90 |
| I-45 | 80 | 80 |
| I-46 | 80 | 90 |
| I-51 | 80 | 90 |
| I-65 | 80 | 80 |
| I-74 | 80 | 90 |
| I-75 | 80 | 90 |
| I-76 | 80 | 90 |
| I-77 | 80 | 80 |
| I-79 | 80 | 80 |
| I-81 | 80 | 80 |
| I-83 | 80 | 90 |
| I-84 | 80 | 90 |
| I-85 | 80 | 100 |

TABLE A8b
Pre-emergence action at 320 g/ha against MATIN in %

| Example number | Dosage [g/ha] | MATIN |
|---|---|---|
| I-01 | 320 | 100 |
| I-02 | 320 | 100 |
| I-03 | 320 | 100 |
| I-04 | 320 | 90 |
| I-05 | 320 | 90 |
| I-07 | 320 | 100 |
| I-08 | 320 | 100 |
| I-10 | 320 | 100 |
| I-100 | 320 | 80 |
| I-107 | 320 | 90 |
| I-11 | 320 | 90 |
| I-111 | 320 | 80 |
| I-116 | 320 | 90 |
| I-118 | 320 | 90 |
| I-119 | 320 | 90 |
| I-121 | 320 | 80 |
| I-122 | 320 | 80 |
| I-125 | 320 | 90 |
| I-126 | 320 | 90 |
| I-13 | 320 | 80 |
| I-148 | 320 | 90 |
| I-149 | 320 | 90 |
| I-15 | 320 | 100 |
| I-151 | 320 | 90 |
| I-152 | 320 | 90 |
| I-153 | 320 | 80 |
| I-154 | 320 | 100 |
| I-155 | 320 | 90 |
| I-156 | 320 | 80 |
| I-159 | 320 | 90 |
| I-16 | 320 | 100 |
| I-160 | 320 | 80 |
| I-163 | 320 | 90 |
| I-164 | 320 | 100 |
| I-167 | 320 | 90 |
| I-17 | 320 | 100 |
| I-171 | 320 | 90 |
| I-174 | 320 | 90 |
| I-175 | 320 | 90 |
| I-18 | 320 | 100 |
| I-180 | 320 | 90 |
| I-183 | 320 | 90 |
| I-184 | 320 | 90 |
| I-185 | 320 | 90 |
| I-187 | 320 | 90 |
| I-188 | 320 | 90 |
| I-189 | 320 | 100 |
| I-19 | 320 | 90 |
| I-190 | 320 | 90 |
| I-191 | 320 | 90 |
| I-192 | 320 | 90 |
| I-193 | 320 | 90 |
| I-194 | 320 | 90 |
| I-195 | 320 | 90 |
| I-196 | 320 | 90 |
| I-198 | 320 | 90 |
| I-199 | 320 | 100 |
| I-20 | 320 | 100 |
| I-201 | 320 | 80 |
| I-202 | 320 | 80 |
| I-204 | 320 | 90 |
| I-206 | 320 | 90 |
| I-207 | 320 | 90 |
| I-208 | 320 | 90 |
| I-210 | 320 | 90 |
| I-211 | 320 | 90 |
| I-212 | 320 | 90 |
| I-216 | 320 | 90 |
| I-217 | 320 | 90 |
| I-218 | 320 | 100 |
| I-221 | 320 | 90 |
| I-225 | 320 | 80 |
| I-229 | 320 | 80 |
| I-23 | 320 | 90 |
| I-231 | 320 | 90 |
| I-24 | 320 | 90 |
| I-247 | 320 | 90 |
| I-25 | 320 | 90 |
| I-27 | 320 | 80 |
| I-276 | 320 | 80 |
| I-278 | 320 | 80 |
| I-279 | 320 | 80 |
| I-28 | 320 | 90 |
| I-29 | 320 | 100 |
| I-291 | 320 | 90 |
| I-293 | 320 | 80 |
| I-296 | 320 | 90 |
| I-298 | 320 | 90 |
| I-30 | 320 | 90 |
| I-300 | 320 | 80 |
| I-302 | 320 | 90 |
| I-305 | 320 | 90 |
| I-306 | 320 | 90 |
| I-307 | 320 | 100 |
| I-308 | 320 | 100 |
| I-309 | 320 | 90 |
| I-311 | 320 | 90 |
| I-312 | 320 | 90 |
| I-313 | 320 | 90 |
| I-315 | 320 | 90 |
| I-316 | 320 | 100 |
| I-317 | 320 | 100 |
| I-319 | 320 | 90 |
| I-32 | 320 | 90 |
| I-320 | 320 | 80 |
| I-321 | 320 | 100 |
| I-322 | 320 | 80 |
| I-323 | 320 | 100 |
| I-324 | 320 | 90 |
| I-325 | 320 | 100 |
| I-327 | 320 | 80 |
| I-33 | 320 | 90 |
| I-330 | 320 | 90 |
| I-331 | 320 | 100 |
| I-332 | 320 | 90 |
| I-333 | 320 | 90 |
| I-334 | 320 | 100 |
| I-336 | 320 | 90 |
| I-337 | 320 | 90 |
| I-338 | 320 | 80 |
| I-339 | 320 | 100 |
| I-34 | 320 | 90 |
| I-341 | 320 | 100 |
| I-342 | 320 | 100 |
| I-343 | 320 | 100 |
| I-347 | 320 | 80 |
| I-348 | 320 | 90 |
| I-35 | 320 | 100 |
| I-350 | 320 | 90 |
| I-351 | 320 | 80 |
| I-352 | 320 | 90 |
| I-354 | 320 | 90 |
| I-359 | 320 | 90 |
| I-36 | 320 | 100 |
| I-360 | 320 | 90 |
| I-369 | 320 | 90 |
| I-37 | 320 | 100 |
| I-370 | 320 | 100 |
| I-373 | 320 | 90 |
| I-374 | 320 | 90 |
| I-375 | 320 | 100 |
| I-376 | 320 | 80 |
| I-377 | 320 | 90 |
| I-38 | 320 | 100 |
| I-388 | 320 | 90 |
| I-39 | 320 | 100 |
| I-41 | 320 | 80 |
| I-42 | 320 | 100 |
| I-43 | 320 | 80 |
| I-44 | 320 | 90 |
| I-46 | 320 | 90 |
| I-48 | 320 | 100 |
| I-51 | 320 | 90 |
| I-52 | 320 | 90 |
| I-53 | 320 | 90 |
| I-54 | 320 | 100 |
| I-56 | 320 | 100 |
| I-64 | 320 | 90 |
| I-65 | 320 | 90 |
| I-66 | 320 | 90 |
| I-69 | 320 | 90 |
| I-73 | 320 | 90 |
| I-74 | 320 | 90 |
| I-75 | 320 | 90 |
| I-76 | 320 | 90 |
| I-77 | 320 | 90 |
| I-79 | 320 | 90 |
| I-80 | 320 | 90 |
| I-81 | 320 | 90 |
| I-82 | 320 | 90 |
| I-83 | 320 | 100 |
| I-84 | 320 | 90 |
| I-85 | 320 | 100 |
| I-86 | 320 | 100 |
| I-87 | 320 | 90 |
| I-88 | 320 | 90 |
| I-90 | 320 | 90 |
| I-91 | 320 | 90 |
| I-93 | 320 | 100 |
| I-94 | 320 | 100 |
| I-95 | 320 | 80 |
| I-96 | 320 | 100 |
| I-97 | 320 | 80 |
| I-98 | 320 | 90 |
| I-99 | 320 | 90 |
| III-22 | 320 | 100 |
| III-24 | 320 | 100 |

TABLE A9a
Pre-emergence action at 80 g/ha against PHBPU in %

| Example number | Dosage [g/ha] | PHBPU |
|---|---|---|
| I-10 | 80 | 90 |
| I-149 | 80 | 80 |
| I-151 | 80 | 80 |
| I-154 | 80 | 80 |
| I-155 | 80 | 90 |
| I-159 | 80 | 80 |
| I-160 | 80 | 80 |
| I-163 | 80 | 90 |
| I-164 | 80 | 80 |
| I-167 | 80 | 90 |
| I-171 | 80 | 80 |
| I-174 | 80 | 90 |
| I-175 | 80 | 80 |
| I-180 | 80 | 90 |
| I-184 | 80 | 80 |
| I-185 | 80 | 80 |
| I-187 | 80 | 90 |
| I-188 | 80 | 80 |
| I-189 | 80 | 90 |
| I-190 | 80 | 90 |
| I-191 | 80 | 80 |
| I-192 | 80 | 90 |
| I-193 | 80 | 80 |
| I-194 | 80 | 90 |
| I-196 | 80 | 90 |
| I-198 | 80 | 80 |
| I-199 | 80 | 80 |
| I-204 | 80 | 90 |
| I-207 | 80 | 90 |
| I-208 | 80 | 90 |
| I-210 | 80 | 90 |
| I-211 | 80 | 90 |
| I-212 | 80 | 90 |
| I-216 | 80 | 90 |
| I-218 | 80 | 90 |
| I-221 | 80 | 90 |
| I-231 | 80 | 90 |
| I-308 | 80 | 90 |
| I-315 | 80 | 90 |
| I-317 | 80 | 90 |
| I-321 | 80 | 80 |
| I-337 | 80 | 80 |
| I-342 | 80 | 80 |
| I-343 | 80 | 80 |
| I-360 | 80 | 80 |
| I-375 | 80 | 80 |
| I-388 | 80 | 90 |
| I-64 | 80 | 90 |
| I-74 | 80 | 90 |
| I-75 | 80 | 90 |
| I-77 | 80 | 80 |
| I-79 | 80 | 90 |
| I-81 | 80 | 90 |
| I-83 | 80 | 80 |
| I-84 | 80 | 90 |
| I-85 | 80 | 90 |
| I-96 | 80 | 90 |

TABLE A9b
Pre-emergence action at 320 g/ha against PHBPU in %

| Example number | Dosage [g/ha] | PHBPU |
|---|---|---|
| I-02 | 320 | 90 |
| I-03 | 320 | 90 |
| I-07 | 320 | 90 |
| I-08 | 320 | 80 |
| I-10 | 320 | 90 |
| I-100 | 320 | 90 |
| I-102 | 320 | 80 |
| I-103 | 320 | 80 |
| I-116 | 320 | 90 |
| I-118 | 320 | 90 |
| I-122 | 320 | 90 |
| I-125 | 320 | 90 |
| I-148 | 320 | 90 |
| I-149 | 320 | 90 |
| I-15 | 320 | 80 |
| I-151 | 320 | 80 |
| I-153 | 320 | 80 |
| I-154 | 320 | 90 |
| I-155 | 320 | 90 |
| I-156 | 320 | 80 |
| I-157 | 320 | 90 |
| I-159 | 320 | 90 |
| I-16 | 320 | 80 |
| I-160 | 320 | 90 |
| I-162 | 320 | 90 |
| I-163 | 320 | 90 |
| I-164 | 320 | 90 |
| I-167 | 320 | 90 |
| I-17 | 320 | 90 |
| I-171 | 320 | 90 |
| I-174 | 320 | 90 |
| I-175 | 320 | 90 |
| I-18 | 320 | 90 |
| I-180 | 320 | 90 |
| I-184 | 320 | 90 |
| I-185 | 320 | 90 |
| I-186 | 320 | 90 |
| I-187 | 320 | 90 |
| I-188 | 320 | 90 |
| I-189 | 320 | 90 |
| I-19 | 320 | 90 |
| I-190 | 320 | 90 |
| I-191 | 320 | 90 |
| I-192 | 320 | 90 |
| I-193 | 320 | 90 |
| I-194 | 320 | 90 |
| I-195 | 320 | 90 |
| I-196 | 320 | 90 |
| I-198 | 320 | 90 |
| I-199 | 320 | 90 |
| I-20 | 320 | 90 |
| I-202 | 320 | 90 |
| I-204 | 320 | 90 |
| I-206 | 320 | 80 |
| I-207 | 320 | 90 |
| I-208 | 320 | 90 |
| I-210 | 320 | 90 |
| I-211 | 320 | 90 |
| I-212 | 320 | 90 |
| I-216 | 320 | 90 |
| I-217 | 320 | 90 |
| I-218 | 320 | 90 |
| I-221 | 320 | 90 |
| I-223 | 320 | 80 |
| I-23 | 320 | 90 |
| I-230 | 320 | 90 |
| I-231 | 320 | 90 |
| I-247 | 320 | 80 |
| I-276 | 320 | 80 |
| I-277 | 320 | 80 |
| I-278 | 320 | 80 |
| I-279 | 320 | 100 |
| I-291 | 320 | 80 |
| I-293 | 320 | 80 |
| I-296 | 320 | 90 |
| I-30 | 320 | 90 |
| I-300 | 320 | 80 |
| I-307 | 320 | 90 |
| I-308 | 320 | 90 |
| I-313 | 320 | 80 |
| I-315 | 320 | 90 |
| I-316 | 320 | 90 |
| I-317 | 320 | 90 |
| I-319 | 320 | 90 |
| I-32 | 320 | 80 |
| I-321 | 320 | 90 |
| I-322 | 320 | 90 |
| I-323 | 320 | 90 |
| I-324 | 320 | 90 |
| I-325 | 320 | 80 |
| I-327 | 320 | 80 |
| I-33 | 320 | 90 |
| I-330 | 320 | 90 |
| I-331 | 320 | 90 |
| I-332 | 320 | 100 |
| I-334 | 320 | 90 |
| I-335 | 320 | 80 |
| I-336 | 320 | 80 |
| I-337 | 320 | 80 |
| I-338 | 320 | 80 |
| I-340 | 320 | 80 |
| I-341 | 320 | 90 |
| I-342 | 320 | 90 |
| I-343 | 320 | 90 |
| I-348 | 320 | 90 |
| I-35 | 320 | 90 |
| I-350 | 320 | 80 |
| I-352 | 320 | 80 |
| I-358 | 320 | 80 |
| I-36 | 320 | 100 |
| I-360 | 320 | 90 |
| I-369 | 320 | 90 |
| I-37 | 320 | 90 |
| I-370 | 320 | 90 |
| I-374 | 320 | 90 |
| I-375 | 320 | 90 |
| I-376 | 320 | 90 |
| I-38 | 320 | 90 |
| I-388 | 320 | 90 |
| I-40 | 320 | 80 |
| I-41 | 320 | 90 |
| I-42 | 320 | 90 |
| I-43 | 320 | 80 |
| I-51 | 320 | 90 |
| I-52 | 320 | 90 |
| I-56 | 320 | 90 |
| I-64 | 320 | 90 |
| I-73 | 320 | 90 |
| I-74 | 320 | 90 |
| I-75 | 320 | 90 |
| I-76 | 320 | 90 |
| I-77 | 320 | 90 |
| I-79 | 320 | 90 |
| I-80 | 320 | 90 |
| I-81 | 320 | 100 |
| I-83 | 320 | 90 |
| I-84 | 320 | 90 |
| I-85 | 320 | 100 |
| I-86 | 320 | 90 |
| I-87 | 320 | 90 |
| I-88 | 320 | 80 |
| I-90 | 320 | 90 |
| I-93 | 320 | 80 |
| I-96 | 320 | 90 |
| I-97 | 320 | 90 |
| I-98 | 320 | 90 |
| III-24 | 320 | 100 |

TABLE A10a
Pre-emergence action at 80 g/ha against POLCO in %

| Example number | Dosage [g/ha] | POLCO |
|---|---|---|
| I-02 | 80 | 80 |
| I-03 | 80 | 90 |
| I-05 | 80 | 90 |
| I-07 | 80 | 90 |
| I-08 | 80 | 80 |
| I-10 | 80 | 100 |
| I-11 | 80 | 90 |
| I-111 | 80 | 80 |
| I-118 | 80 | 80 |
| I-119 | 80 | 80 |
| I-148 | 80 | 80 |
| I-149 | 80 | 80 |
| I-15 | 80 | 90 |
| I-155 | 80 | 90 |
| I-159 | 80 | 90 |
| I-16 | 80 | 90 |
| I-163 | 80 | 80 |
| I-167 | 80 | 90 |
| I-17 | 80 | 90 |
| I-171 | 80 | 90 |
| I-174 | 80 | 90 |
| I-175 | 80 | 90 |
| I-18 | 80 | 100 |
| I-180 | 80 | 90 |
| I-183 | 80 | 80 |
| I-184 | 80 | 90 |
| I-185 | 80 | 80 |
| I-187 | 80 | 90 |
| I-188 | 80 | 90 |
| I-189 | 80 | 100 |
| I-19 | 80 | 90 |
| I-190 | 80 | 90 |
| I-191 | 80 | 80 |
| I-192 | 80 | 90 |
| I-193 | 80 | 80 |
| I-194 | 80 | 80 |
| I-195 | 80 | 90 |
| I-196 | 80 | 90 |
| I-198 | 80 | 90 |
| I-199 | 80 | 80 |
| I-20 | 80 | 90 |
| I-204 | 80 | 90 |
| I-206 | 80 | 80 |
| I-207 | 80 | 90 |
| I-208 | 80 | 90 |
| I-210 | 80 | 90 |
| I-211 | 80 | 90 |
| I-212 | 80 | 90 |
| I-216 | 80 | 90 |
| I-218 | 80 | 90 |
| I-221 | 80 | 80 |
| I-230 | 80 | 80 |
| I-24 | 80 | 90 |
| I-25 | 80 | 80 |
| I-27 | 80 | 90 |
| I-28 | 80 | 80 |
| I-291 | 80 | 80 |
| I-296 | 80 | 80 |
| I-30 | 80 | 80 |
| I-307 | 80 | 90 |
| I-308 | 80 | 100 |
| I-311 | 80 | 80 |
| I-313 | 80 | 80 |
| I-315 | 80 | 80 |
| I-316 | 80 | 100 |
| I-317 | 80 | 100 |
| I-32 | 80 | 90 |
| I-321 | 80 | 90 |
| I-322 | 80 | 80 |
| I-324 | 80 | 90 |
| I-33 | 80 | 90 |
| I-331 | 80 | 100 |
| I-332 | 80 | 80 |
| I-333 | 80 | 90 |
| I-336 | 80 | 80 |
| I-337 | 80 | 80 |
| I-338 | 80 | 100 |
| I-339 | 80 | 90 |
| I-34 | 80 | 80 |
| I-340 | 80 | 80 |
| I-341 | 80 | 90 |
| I-342 | 80 | 90 |
| I-343 | 80 | 90 |
| I-350 | 80 | 90 |
| I-351 | 80 | 80 |
| I-352 | 80 | 80 |
| I-358 | 80 | 80 |
| I-36 | 80 | 80 |
| I-37 | 80 | 80 |
| I-370 | 80 | 80 |
| I-374 | 80 | 80 |
| I-375 | 80 | 90 |
| I-38 | 80 | 90 |
| I-388 | 80 | 90 |
| I-41 | 80 | 80 |
| I-44 | 80 | 90 |
| I-45 | 80 | 90 |
| I-47 | 80 | 80 |
| I-48 | 80 | 90 |
| I-51 | 80 | 80 |
| I-56 | 80 | 80 |
| I-59 | 80 | 80 |
| I-66 | 80 | 90 |
| I-73 | 80 | 80 |
| I-76 | 80 | 80 |
| I-79 | 80 | 90 |
| I-81 | 80 | 90 |
| I-83 | 80 | 80 |
| I-87 | 80 | 80 |
| I-88 | 80 | 80 |
| I-93 | 80 | 90 |
| I-94 | 80 | 90 |
| I-96 | 80 | 90 |

TABLE A10b
Pre-emergence action at 320 g/ha against POLCO in %

| Example number | Dosage [g/ha] | POLCO |
|---|---|---|
| I-01 | 320 | 90 |
| I-02 | 320 | 80 |
| I-03 | 320 | 100 |
| I-04 | 320 | 100 |
| I-05 | 320 | 90 |
| I-07 | 320 | 100 |
| I-08 | 320 | 100 |
| I-10 | 320 | 100 |
| I-100 | 320 | 90 |
| I-102 | 320 | 90 |
| I-107 | 320 | 80 |
| I-11 | 320 | 90 |
| I-111 | 320 | 100 |
| I-116 | 320 | 90 |
| I-118 | 320 | 90 |
| I-119 | 320 | 90 |
| I-121 | 320 | 100 |
| I-122 | 320 | 80 |
| I-125 | 320 | 90 |
| I-126 | 320 | 90 |
| I-13 | 320 | 100 |
| I-147 | 320 | 90 |
| I-148 | 320 | 90 |
| I-149 | 320 | 90 |
| I-15 | 320 | 100 |
| I-151 | 320 | 90 |
| I-154 | 320 | 90 |
| I-155 | 320 | 90 |
| I-157 | 320 | 90 |
| I-159 | 320 | 90 |
| I-16 | 320 | 90 |
| I-160 | 320 | 90 |
| I-162 | 320 | 100 |
| I-163 | 320 | 80 |
| I-164 | 320 | 100 |
| I-167 | 320 | 90 |
| I-17 | 320 | 100 |
| I-171 | 320 | 90 |
| I-174 | 320 | 100 |
| I-175 | 320 | 90 |
| I-18 | 320 | 100 |
| I-180 | 320 | 100 |
| I-183 | 320 | 90 |
| I-184 | 320 | 90 |
| I-185 | 320 | 90 |
| I-186 | 320 | 80 |
| I-187 | 320 | 90 |
| I-188 | 320 | 90 |
| I-189 | 320 | 100 |
| I-19 | 320 | 100 |
| I-190 | 320 | 90 |
| I-191 | 320 | 90 |
| I-192 | 320 | 100 |
| I-193 | 320 | 100 |
| I-194 | 320 | 100 |
| I-195 | 320 | 100 |
| I-196 | 320 | 90 |
| I-198 | 320 | 90 |
| I-199 | 320 | 90 |
| I-20 | 320 | 100 |
| I-202 | 320 | 90 |
| I-204 | 320 | 90 |
| I-205 | 320 | 80 |
| I-206 | 320 | 80 |
| I-207 | 320 | 100 |
| I-208 | 320 | 90 |
| I-210 | 320 | 100 |
| I-211 | 320 | 90 |
| I-212 | 320 | 90 |
| I-216 | 320 | 90 |
| I-217 | 320 | 80 |
| I-218 | 320 | 90 |
| I-221 | 320 | 90 |
| I-223 | 320 | 80 |
| I-225 | 320 | 90 |
| I-229 | 320 | 90 |
| I-23 | 320 | 90 |
| I-230 | 320 | 90 |
| I-237 | 320 | 80 |
| I-238 | 320 | 80 |
| I-24 | 320 | 90 |
| I-242 | 320 | 80 |
| I-244 | 320 | 80 |
| I-247 | 320 | 90 |
| I-25 | 320 | 90 |
| I-26 | 320 | 90 |
| I-27 | 320 | 100 |
| I-276 | 320 | 80 |
| I-279 | 320 | 80 |
| I-28 | 320 | 90 |
| I-29 | 320 | 90 |
| I-291 | 320 | 90 |
| I-293 | 320 | 80 |
| I-296 | 320 | 90 |
| I-30 | 320 | 90 |
| I-307 | 320 | 100 |
| I-308 | 320 | 100 |
| I-309 | 320 | 80 |
| I-31 | 320 | 80 |
| I-310 | 320 | 90 |
| I-311 | 320 | 90 |
| I-313 | 320 | 80 |
| I-315 | 320 | 90 |
| I-316 | 320 | 100 |
| I-317 | 320 | 100 |
| I-319 | 320 | 90 |
| I-32 | 320 | 90 |
| I-320 | 320 | 90 |
| I-321 | 320 | 100 |
| I-322 | 320 | 90 |
| I-323 | 320 | 100 |
| I-324 | 320 | 100 |
| I-325 | 320 | 90 |
| I-327 | 320 | 90 |
| I-33 | 320 | 100 |
| I-330 | 320 | 90 |
| I-331 | 320 | 100 |
| I-332 | 320 | 80 |
| I-333 | 320 | 90 |
| I-334 | 320 | 90 |
| I-335 | 320 | 90 |
| I-336 | 320 | 90 |
| I-337 | 320 | 90 |
| I-338 | 320 | 100 |
| I-339 | 320 | 100 |
| I-34 | 320 | 90 |
| I-340 | 320 | 90 |
| I-341 | 320 | 100 |
| I-342 | 320 | 100 |
| I-343 | 320 | 100 |
| I-347 | 320 | 80 |
| I-349 | 320 | 80 |
| I-35 | 320 | 90 |
| I-350 | 320 | 90 |
| I-351 | 320 | 80 |
| I-352 | 320 | 90 |
| I-354 | 320 | 80 |
| I-358 | 320 | 90 |
| I-359 | 320 | 80 |
| I-36 | 320 | 90 |
| I-360 | 320 | 100 |
| I-362 | 320 | 80 |
| I-369 | 320 | 90 |
| I-37 | 320 | 90 |
| I-370 | 320 | 100 |
| I-373 | 320 | 90 |
| I-374 | 320 | 90 |
| I-375 | 320 | 90 |
| I-378 | 320 | 80 |
| I-38 | 320 | 100 |
| I-381 | 320 | 80 |
| I-388 | 320 | 90 |
| I-39 | 320 | 90 |
| I-40 | 320 | 80 |
| I-41 | 320 | 90 |
| I-42 | 320 | 90 |
| I-43 | 320 | 80 |
| I-44 | 320 | 90 |
| I-46 | 320 | 80 |
| I-47 | 320 | 90 |
| I-48 | 320 | 90 |
| I-51 | 320 | 90 |
| I-52 | 320 | 90 |
| I-53 | 320 | 90 |
| I-54 | 320 | 90 |
| I-56 | 320 | 90 |
| I-58 | 320 | 90 |
| I-59 | 320 | 90 |
| I-64 | 320 | 90 |
| I-65 | 320 | 90 |
| I-66 | 320 | 90 |
| I-73 | 320 | 90 |
| I-74 | 320 | 100 |
| I-75 | 320 | 90 |
| I-76 | 320 | 90 |
| I-77 | 320 | 90 |
| I-79 | 320 | 90 |
| I-80 | 320 | 90 |
| I-81 | 320 | 90 |
| I-83 | 320 | 90 |
| I-84 | 320 | 90 |
| I-85 | 320 | 100 |
| I-86 | 320 | 100 |
| I-87 | 320 | 90 |
| I-88 | 320 | 90 |
| I-90 | 320 | 90 |
| I-93 | 320 | 90 |
| I-94 | 320 | 90 |
| I-95 | 320 | 90 |
| I-96 | 320 | 90 |
| I-97 | 320 | 90 |
| I-98 | 320 | 90 |
| I-99 | 320 | 90 |
| III-22 | 320 | 100 |
| III-24 | 320 | 100 |

TABLE A11a
Pre-emergence action at 80 g/ha against SETVI in %

| Example number | Dosage [g/ha] | SETVI |
|---|---|---|
| I-02 | 80 | 100 |
| I-05 | 80 | 100 |
| I-08 | 80 | 90 |
| I-10 | 80 | 100 |
| I-100 | 80 | 90 |
| I-101 | 80 | 90 |
| I-102 | 80 | 90 |
| I-11 | 80 | 90 |
| I-111 | 80 | 90 |
| I-116 | 80 | 90 |
| I-118 | 80 | 90 |
| I-122 | 80 | 100 |
| I-148 | 80 | 90 |
| I-149 | 80 | 100 |
| I-15 | 80 | 100 |
| I-151 | 80 | 90 |
| I-154 | 80 | 90 |
| I-155 | 80 | 80 |
| I-159 | 80 | 90 |
| I-16 | 80 | 90 |
| I-163 | 80 | 80 |
| I-164 | 80 | 100 |
| I-167 | 80 | 100 |
| I-17 | 80 | 100 |
| I-171 | 80 | 100 |
| I-174 | 80 | 90 |
| I-175 | 80 | 90 |
| I-18 | 80 | 100 |
| I-180 | 80 | 100 |
| I-184 | 80 | 100 |
| I-185 | 80 | 80 |
| I-187 | 80 | 100 |
| I-188 | 80 | 100 |
| I-189 | 80 | 100 |
| I-19 | 80 | 100 |
| I-190 | 80 | 90 |
| I-191 | 80 | 80 |
| I-192 | 80 | 90 |
| I-193 | 80 | 100 |
| I-194 | 80 | 90 |
| I-195 | 80 | 100 |
| I-196 | 80 | 90 |
| I-198 | 80 | 100 |
| I-199 | 80 | 90 |
| I-204 | 80 | 90 |
| I-207 | 80 | 80 |
| I-208 | 80 | 90 |
| I-210 | 80 | 90 |
| I-211 | 80 | 90 |
| I-212 | 80 | 100 |
| I-216 | 80 | 80 |
| I-218 | 80 | 90 |
| I-221 | 80 | 90 |
| I-231 | 80 | 90 |
| I-29 | 80 | 90 |
| I-291 | 80 | 90 |
| I-296 | 80 | 90 |
| I-298 | 80 | 100 |
| I-30 | 80 | 90 |
| I-307 | 80 | 100 |
| I-313 | 80 | 100 |
| I-316 | 80 | 100 |
| I-317 | 80 | 80 |
| I-32 | 80 | 100 |
| I-322 | 80 | 90 |
| I-33 | 80 | 90 |
| I-334 | 80 | 90 |
| I-337 | 80 | 90 |
| I-338 | 80 | 100 |
| I-339 | 80 | 100 |
| I-34 | 80 | 90 |
| I-341 | 80 | 80 |
| I-342 | 80 | 90 |
| I-343 | 80 | 100 |
| I-348 | 80 | 80 |
| I-35 | 80 | 100 |
| I-350 | 80 | 100 |
| I-359 | 80 | 90 |
| I-36 | 80 | 100 |
| I-360 | 80 | 90 |
| I-37 | 80 | 100 |
| I-370 | 80 | 100 |
| I-375 | 80 | 90 |
| I-38 | 80 | 100 |
| I-388 | 80 | 90 |
| I-39 | 80 | 100 |
| I-40 | 80 | 100 |
| I-41 | 80 | 90 |
| I-42 | 80 | 100 |
| I-43 | 80 | 100 |
| I-44 | 80 | 90 |
| I-46 | 80 | 90 |
| I-48 | 80 | 100 |
| I-51 | 80 | 90 |
| I-53 | 80 | 90 |
| I-59 | 80 | 90 |
| I-64 | 80 | 90 |
| I-66 | 80 | 100 |
| I-73 | 80 | 90 |
| I-74 | 80 | 90 |
| I-76 | 80 | 80 |
| I-79 | 80 | 100 |
| I-80 | 80 | 90 |
| I-81 | 80 | 90 |
| I-83 | 80 | 90 |
| I-85 | 80 | 100 |
| I-86 | 80 | 100 |
| I-87 | 80 | 90 |
| I-90 | 80 | 90 |
| I-91 | 80 | 100 |
| I-96 | 80 | 100 |
| I-97 | 80 | 80 |
| III-22 | 80 | 100 |
| III-24 | 80 | 100 |

TABLE A11b
Pre-emergence action at 320 g/ha against SETVI in %

| Example number | Dosage [g/ha] | SETVI |
|---|---|---|
| I-01 | 320 | 100 |
| I-02 | 320 | 100 |
| I-03 | 320 | 100 |
| I-04 | 320 | 100 |
| I-05 | 320 | 100 |
| I-07 | 320 | 100 |
| I-08 | 320 | 100 |
| I-10 | 320 | 100 |
| I-100 | 320 | 100 |
| I-101 | 320 | 100 |
| I-102 | 320 | 100 |
| I-103 | 320 | 90 |
| I-107 | 320 | 100 |
| I-11 | 320 | 100 |
| I-111 | 320 | 100 |
| I-116 | 320 | 100 |
| I-118 | 320 | 100 |
| I-119 | 320 | 100 |
| I-120 | 320 | 100 |
| I-121 | 320 | 100 |
| I-122 | 320 | 100 |
| I-125 | 320 | 90 |
| I-126 | 320 | 90 |
| I-13 | 320 | 90 |
| I-148 | 320 | 100 |
| I-149 | 320 | 100 |
| I-15 | 320 | 100 |
| I-151 | 320 | 100 |
| I-152 | 320 | 90 |
| I-153 | 320 | 90 |
| I-154 | 320 | 100 |
| I-155 | 320 | 100 |
| I-156 | 320 | 90 |
| I-159 | 320 | 100 |
| I-16 | 320 | 100 |
| I-163 | 320 | 100 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-183 | 320 | 90 |
| I-184 | 320 | 100 |
| I-185 | 320 | 100 |
| I-186 | 320 | 80 |
| I-187 | 320 | 100 |
| I-188 | 320 | 100 |
| I-189 | 320 | 100 |
| I-19 | 320 | 100 |
| I-190 | 320 | 100 |
| I-191 | 320 | 100 |
| I-192 | 320 | 100 |
| I-193 | 320 | 100 |
| I-194 | 320 | 100 |
| I-195 | 320 | 100 |
| I-196 | 320 | 100 |
| I-198 | 320 | 100 |
| I-199 | 320 | 100 |
| I-20 | 320 | 100 |
| I-202 | 320 | 90 |
| I-204 | 320 | 100 |
| I-207 | 320 | 100 |
| I-208 | 320 | 100 |
| I-210 | 320 | 100 |
| I-211 | 320 | 100 |
| I-212 | 320 | 100 |
| I-216 | 320 | 100 |
| I-217 | 320 | 90 |
| I-218 | 320 | 100 |
| I-221 | 320 | 100 |
| I-225 | 320 | 80 |
| I-229 | 320 | 90 |
| I-23 | 320 | 100 |
| I-230 | 320 | 100 |
| I-231 | 320 | 90 |
| I-24 | 320 | 90 |
| I-247 | 320 | 100 |
| I-25 | 320 | 100 |
| I-26 | 320 | 100 |
| I-27 | 320 | 100 |
| I-272 | 320 | 90 |
| I-276 | 320 | 90 |
| I-277 | 320 | 80 |
| I-278 | 320 | 100 |
| I-279 | 320 | 90 |
| I-28 | 320 | 100 |
| I-29 | 320 | 100 |
| I-291 | 320 | 90 |
| I-296 | 320 | 100 |
| I-298 | 320 | 100 |
| I-30 | 320 | 100 |
| I-300 | 320 | 90 |
| I-302 | 320 | 100 |
| I-305 | 320 | 90 |
| I-306 | 320 | 80 |
| I-307 | 320 | 100 |
| I-308 | 320 | 100 |
| I-309 | 320 | 90 |
| I-31 | 320 | 90 |
| I-311 | 320 | 100 |
| I-312 | 320 | 90 |
| I-313 | 320 | 100 |
| I-315 | 320 | 100 |
| I-316 | 320 | 100 |
| I-317 | 320 | 90 |
| I-319 | 320 | 100 |
| I-32 | 320 | 100 |
| I-320 | 320 | 90 |
| I-321 | 320 | 100 |
| I-322 | 320 | 100 |
| I-323 | 320 | 100 |
| I-324 | 320 | 100 |
| I-327 | 320 | 100 |
| I-33 | 320 | 100 |
| I-330 | 320 | 100 |
| I-331 | 320 | 90 |
| I-332 | 320 | 100 |
| I-333 | 320 | 90 |
| I-334 | 320 | 90 |
| I-336 | 320 | 100 |
| I-337 | 320 | 100 |
| I-338 | 320 | 100 |
| I-339 | 320 | 100 |
| I-34 | 320 | 100 |
| I-341 | 320 | 100 |
| I-342 | 320 | 100 |
| I-343 | 320 | 100 |
| I-347 | 320 | 80 |
| I-348 | 320 | 100 |
| I-35 | 320 | 100 |
| I-350 | 320 | 100 |
| I-351 | 320 | 100 |
| I-352 | 320 | 90 |
| I-354 | 320 | 90 |
| I-358 | 320 | 100 |
| I-359 | 320 | 100 |
| I-36 | 320 | 100 |
| I-360 | 320 | 100 |
| I-362 | 320 | 80 |
| I-369 | 320 | 90 |
| I-37 | 320 | 100 |
| I-370 | 320 | 100 |
| I-373 | 320 | 80 |
| I-374 | 320 | 100 |
| I-375 | 320 | 100 |
| I-376 | 320 | 100 |
| I-377 | 320 | 90 |
| I-38 | 320 | 100 |
| I-388 | 320 | 100 |
| I-39 | 320 | 100 |
| I-40 | 320 | 100 |
| I-41 | 320 | 100 |
| I-42 | 320 | 100 |
| I-43 | 320 | 100 |
| I-44 | 320 | 100 |
| I-46 | 320 | 100 |
| I-47 | 320 | 100 |
| I-48 | 320 | 100 |
| I-51 | 320 | 100 |
| I-52 | 320 | 100 |
| I-53 | 320 | 100 |
| I-54 | 320 | 100 |
| I-56 | 320 | 100 |
| I-59 | 320 | 100 |
| I-60 | 320 | 80 |
| I-64 | 320 | 100 |
| I-65 | 320 | 90 |
| I-66 | 320 | 100 |
| I-69 | 320 | 100 |
| I-70 | 320 | 90 |
| I-71 | 320 | 100 |
| I-72 | 320 | 90 |
| I-73 | 320 | 100 |
| I-74 | 320 | 100 |
| I-75 | 320 | 100 |
| I-76 | 320 | 100 |
| I-77 | 320 | 100 |
| I-78 | 320 | 80 |
| I-79 | 320 | 100 |
| I-80 | 320 | 100 |
| I-81 | 320 | 100 |
| I-82 | 320 | 90 |
| I-83 | 320 | 100 |
| I-84 | 320 | 100 |
| I-85 | 320 | 100 |
| I-86 | 320 | 100 |
| I-87 | 320 | 100 |
| I-88 | 320 | 100 |
| I-90 | 320 | 100 |
| I-91 | 320 | 100 |
| I-93 | 320 | 100 |
| I-94 | 320 | 100 |
| I-95 | 320 | 100 |
| I-96 | 320 | 100 |
| I-97 | 320 | 100 |
| I-98 | 320 | 100 |
| I-99 | 320 | 90 |
| II-36 | 320 | 80 |
| III-02 | 320 | 80 |
| III-22 | 320 | 100 |
| III-23 | 320 | 100 |
| III-24 | 320 | 100 |
| III-27 | 320 | 90 |

TABLE A12a
Pre-emergence action at 80 g/ha against VERPE in %

| Example number | Dosage [g/ha] | VERPE |
|---|---|---|
| I-03 | 80 | 90 |
| I-05 | 80 | 100 |
| I-10 | 80 | 80 |
| I-110 | 80 | 100 |
| I-116 | 80 | 90 |
| I-148 | 80 | 90 |
| I-162 | 80 | 90 |
| I-164 | 80 | 90 |
| I-167 | 80 | 100 |
| I-171 | 80 | 100 |
| I-174 | 80 | 100 |
| I-175 | 80 | 100 |
| I-18 | 80 | 90 |
| I-180 | 80 | 100 |
| I-184 | 80 | 90 |
| I-185 | 80 | 80 |
| I-186 | 80 | 90 |
| I-187 | 80 | 90 |
| I-188 | 80 | 100 |
| I-189 | 80 | 90 |
| I-190 | 80 | 80 |
| I-191 | 80 | 100 |
| I-193 | 80 | 80 |
| I-196 | 80 | 90 |
| I-198 | 80 | 90 |
| I-199 | 80 | 90 |
| I-202 | 80 | 80 |
| I-208 | 80 | 90 |
| I-212 | 80 | 80 |
| I-218 | 80 | 90 |
| I-225 | 80 | 80 |
| I-231 | 80 | 80 |
| I-24 | 80 | 80 |
| I-27 | 80 | 80 |
| I-296 | 80 | 90 |
| I-307 | 80 | 90 |
| I-308 | 80 | 100 |
| I-311 | 80 | 80 |
| I-313 | 80 | 80 |
| I-317 | 80 | 80 |
| I-319 | 80 | 80 |
| I-32 | 80 | 80 |
| I-331 | 80 | 100 |
| I-332 | 80 | 80 |
| I-336 | 80 | 80 |
| I-337 | 80 | 90 |
| I-34 | 80 | 90 |
| I-342 | 80 | 90 |
| I-350 | 80 | 90 |
| I-369 | 80 | 90 |
| I-37 | 80 | 80 |
| I-370 | 80 | 90 |
| I-375 | 80 | 100 |
| I-376 | 80 | 80 |
| I-388 | 80 | 90 |
| I-41 | 80 | 90 |
| I-51 | 80 | 100 |
| I-52 | 80 | 80 |
| I-76 | 80 | 100 |
| I-79 | 80 | 80 |
| I-83 | 80 | 90 |
| I-84 | 80 | 90 |
| I-85 | 80 | 100 |
| I-87 | 80 | 80 |
| I-88 | 80 | 80 |
| I-96 | 80 | 90 |

TABLE A12b
Pre-emergence action at 320 g/ha against VERPE in %

| Example number | Dosage [g/ha] | VERPE |
|---|---|---|
| I-01 | 320 | 90 |
| I-02 | 320 | 90 |
| I-03 | 320 | 100 |
| I-04 | 320 | 100 |
| I-05 | 320 | 100 |
| I-07 | 320 | 100 |
| I-08 | 320 | 100 |
| I-10 | 320 | 100 |
| I-100 | 320 | 100 |
| I-101 | 320 | 100 |
| I-102 | 320 | 80 |
| I-103 | 320 | 90 |
| I-11 | 320 | 90 |
| I-111 | 320 | 90 |
| I-112 | 320 | 80 |
| I-116 | 320 | 90 |
| I-118 | 320 | 90 |
| I-120 | 320 | 90 |
| I-121 | 320 | 100 |
| I-122 | 320 | 100 |
| I-126 | 320 | 80 |
| I-13 | 320 | 80 |
| I-148 | 320 | 100 |
| I-149 | 320 | 100 |
| I-15 | 320 | 100 |
| I-154 | 320 | 100 |
| I-155 | 320 | 100 |
| I-157 | 320 | 90 |
| I-159 | 320 | 100 |
| I-16 | 320 | 90 |
| I-160 | 320 | 90 |
| I-161 | 320 | 80 |
| I-162 | 320 | 90 |
| I-163 | 320 | 80 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-184 | 320 | 100 |
| I-185 | 320 | 90 |
| I-186 | 320 | 90 |
| I-187 | 320 | 90 |
| I-188 | 320 | 100 |
| I-189 | 320 | 100 |
| I-19 | 320 | 100 |
| I-190 | 320 | 100 |
| I-191 | 320 | 100 |
| I-192 | 320 | 90 |
| I-193 | 320 | 90 |
| I-194 | 320 | 90 |
| I-195 | 320 | 100 |
| I-196 | 320 | 100 |
| I-198 | 320 | 100 |
| I-199 | 320 | 100 |
| I-20 | 320 | 100 |
| I-202 | 320 | 90 |
| I-204 | 320 | 100 |
| I-205 | 320 | 100 |
| I-207 | 320 | 90 |
| I-208 | 320 | 100 |
| I-210 | 320 | 100 |
| I-211 | 320 | 100 |
| I-212 | 320 | 100 |
| I-216 | 320 | 90 |
| I-218 | 320 | 100 |
| I-221 | 320 | 90 |
| I-223 | 320 | 80 |
| I-225 | 320 | 90 |
| I-23 | 320 | 90 |
| I-231 | 320 | 80 |
| I-235 | 320 | 80 |
| I-24 | 320 | 100 |
| I-246 | 320 | 80 |
| I-247 | 320 | 90 |
| I-25 | 320 | 90 |
| I-27 | 320 | 100 |
| I-279 | 320 | 80 |
| I-28 | 320 | 100 |
| I-29 | 320 | 90 |
| I-291 | 320 | 80 |
| I-296 | 320 | 100 |
| I-298 | 320 | 100 |
| I-30 | 320 | 90 |
| I-302 | 320 | 90 |
| I-307 | 320 | 100 |
| I-308 | 320 | 100 |
| I-31 | 320 | 90 |
| I-311 | 320 | 90 |
| I-313 | 320 | 90 |
| I-315 | 320 | 100 |
| I-316 | 320 | 100 |
| I-317 | 320 | 100 |
| I-319 | 320 | 90 |
| I-32 | 320 | 100 |
| I-321 | 320 | 100 |
| I-323 | 320 | 100 |
| I-324 | 320 | 100 |
| I-33 | 320 | 100 |
| I-330 | 320 | 90 |
| I-331 | 320 | 100 |
| I-332 | 320 | 90 |
| I-333 | 320 | 100 |
| I-334 | 320 | 80 |
| I-335 | 320 | 80 |
| I-336 | 320 | 90 |
| I-337 | 320 | 90 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 100 |
| I-348 | 320 | 90 |
| I-35 | 320 | 100 |
| I-350 | 320 | 100 |
| I-351 | 320 | 80 |
| I-352 | 320 | 80 |
| I-36 | 320 | 100 |
| I-360 | 320 | 90 |
| I-369 | 320 | 90 |
| I-37 | 320 | 100 |
| I-370 | 320 | 100 |
| I-374 | 320 | 100 |
| I-375 | 320 | 100 |
| I-376 | 320 | 80 |
| I-377 | 320 | 80 |
| I-38 | 320 | 100 |
| I-388 | 320 | 100 |
| I-39 | 320 | 100 |
| I-40 | 320 | 100 |
| I-41 | 320 | 100 |
| I-42 | 320 | 100 |
| I-43 | 320 | 90 |
| I-44 | 320 | 80 |
| I-46 | 320 | 100 |
| I-47 | 320 | 90 |
| I-48 | 320 | 100 |
| I-51 | 320 | 100 |
| I-52 | 320 | 100 |
| I-53 | 320 | 100 |
| I-54 | 320 | 100 |
| I-56 | 320 | 100 |
| I-59 | 320 | 90 |
| I-64 | 320 | 90 |
| I-65 | 320 | 80 |
| I-66 | 320 | 100 |
| I-70 | 320 | 80 |
| I-73 | 320 | 100 |
| I-74 | 320 | 100 |
| I-75 | 320 | 90 |
| I-76 | 320 | 100 |
| I-77 | 320 | 90 |
| I-79 | 320 | 100 |
| I-80 | 320 | 90 |
| I-81 | 320 | 90 |
| I-83 | 320 | 100 |
| I-84 | 320 | 100 |
| I-85 | 320 | 100 |
| I-86 | 320 | 90 |
| I-87 | 320 | 90 |
| I-88 | 320 | 90 |
| I-90 | 320 | 90 |
| I-91 | 320 | 100 |
| I-93 | 320 | 100 |
| I-94 | 320 | 90 |
| I-95 | 320 | 100 |
| I-96 | 320 | 90 |
| I-97 | 320 | 90 |
| I-98 | 320 | 100 |
| I-99 | 320 | 90 |
| II-36 | 320 | 80 |
| III-22 | 320 | 100 |
| III-24 | 320 | 100 |
| III-28 | 320 | 80 |

TABLE A13a
Pre-emergence action at 80 g/ha against VIOTR in %

| Example number | Dosage [g/ha] | VIOTR |
|---|---|---|
| I-02 | 80 | 100 |
| I-03 | 80 | 100 |
| I-05 | 80 | 90 |
| I-07 | 80 | 90 |
| I-08 | 80 | 100 |
| I-10 | 80 | 100 |
| I-101 | 80 | 80 |
| I-102 | 80 | 90 |
| I-111 | 80 | 80 |
| I-114 | 80 | 80 |
| I-119 | 80 | 80 |
| I-122 | 80 | 100 |
| I-126 | 80 | 100 |
| I-148 | 80 | 100 |
| I-149 | 80 | 100 |
| I-15 | 80 | 100 |
| I-152 | 80 | 80 |
| I-153 | 80 | 90 |
| I-154 | 80 | 100 |
| I-155 | 80 | 100 |
| I-156 | 80 | 80 |
| I-157 | 80 | 80 |
| I-159 | 80 | 100 |
| I-16 | 80 | 100 |
| I-160 | 80 | 90 |
| I-162 | 80 | 80 |
| I-163 | 80 | 90 |
| I-164 | 80 | 100 |
| I-167 | 80 | 100 |
| I-17 | 80 | 100 |
| I-171 | 80 | 100 |
| I-174 | 80 | 100 |
| I-175 | 80 | 100 |
| I-18 | 80 | 100 |
| I-180 | 80 | 100 |
| I-183 | 80 | 80 |
| I-184 | 80 | 100 |
| I-185 | 80 | 100 |
| I-186 | 80 | 80 |
| I-187 | 80 | 100 |
| I-188 | 80 | 100 |
| I-189 | 80 | 100 |
| I-19 | 80 | 100 |
| I-190 | 80 | 100 |
| I-191 | 80 | 100 |
| I-192 | 80 | 80 |
| I-193 | 80 | 100 |
| I-195 | 80 | 80 |
| I-196 | 80 | 90 |
| I-198 | 80 | 90 |
| I-199 | 80 | 100 |
| I-20 | 80 | 100 |
| I-202 | 80 | 90 |
| I-204 | 80 | 100 |
| I-206 | 80 | 90 |
| I-207 | 80 | 100 |
| I-208 | 80 | 100 |
| I-210 | 80 | 90 |
| I-211 | 80 | 90 |
| I-212 | 80 | 100 |
| I-216 | 80 | 100 |
| I-218 | 80 | 100 |
| I-221 | 80 | 90 |
| I-223 | 80 | 90 |
| I-225 | 80 | 90 |
| I-23 | 80 | 90 |
| I-230 | 80 | 80 |
| I-231 | 80 | 100 |
| I-24 | 80 | 90 |
| I-25 | 80 | 100 |
| I-26 | 80 | 100 |
| I-27 | 80 | 100 |
| I-28 | 80 | 100 |
| I-29 | 80 | 90 |
| I-291 | 80 | 90 |
| I-293 | 80 | 80 |
| I-296 | 80 | 90 |
| I-298 | 80 | 100 |
| I-30 | 80 | 90 |
| I-307 | 80 | 100 |
| I-308 | 80 | 100 |
| I-310 | 80 | 90 |
| I-311 | 80 | 90 |
| I-315 | 80 | 90 |
| I-316 | 80 | 100 |
| I-317 | 80 | 100 |
| I-319 | 80 | 100 |
| I-32 | 80 | 100 |
| I-321 | 80 | 90 |
| I-322 | 80 | 80 |
| I-323 | 80 | 100 |
| I-324 | 80 | 100 |
| I-325 | 80 | 90 |
| I-327 | 80 | 100 |
| I-33 | 80 | 100 |
| I-330 | 80 | 90 |
| I-331 | 80 | 100 |
| I-332 | 80 | 90 |
| I-333 | 80 | 100 |
| I-336 | 80 | 90 |
| I-337 | 80 | 80 |
| I-338 | 80 | 100 |
| I-339 | 80 | 100 |
| I-34 | 80 | 100 |
| I-340 | 80 | 100 |
| I-341 | 80 | 100 |
| I-342 | 80 | 100 |
| I-343 | 80 | 100 |
| I-347 | 80 | 90 |
| I-348 | 80 | 80 |
| I-35 | 80 | 100 |
| I-350 | 80 | 100 |
| I-358 | 80 | 80 |
| I-36 | 80 | 100 |
| I-369 | 80 | 80 |
| I-37 | 80 | 100 |
| I-374 | 80 | 90 |
| I-375 | 80 | 100 |
| I-38 | 80 | 100 |
| I-381 | 80 | 80 |
| I-388 | 80 | 100 |
| I-39 | 80 | 100 |
| I-40 | 80 | 90 |
| I-41 | 80 | 100 |
| I-42 | 80 | 100 |
| I-43 | 80 | 100 |
| I-44 | 80 | 100 |
| I-45 | 80 | 90 |
| I-46 | 80 | 90 |
| I-47 | 80 | 100 |
| I-48 | 80 | 100 |
| I-51 | 80 | 100 |
| I-52 | 80 | 100 |
| I-53 | 80 | 90 |
| I-54 | 80 | 100 |
| I-56 | 80 | 100 |
| I-59 | 80 | 90 |
| I-66 | 80 | 90 |
| I-73 | 80 | 100 |
| I-74 | 80 | 90 |
| I-76 | 80 | 100 |
| I-77 | 80 | 90 |
| I-79 | 80 | 100 |
| I-81 | 80 | 80 |
| I-83 | 80 | 100 |
| I-84 | 80 | 90 |
| I-85 | 80 | 100 |
| I-86 | 80 | 80 |
| I-91 | 80 | 90 |
| I-94 | 80 | 100 |
| I-95 | 80 | 100 |
| II-36 | 80 | 80 |
| III-22 | 80 | 90 |
| III-24 | 80 | 100 |

TABLE A13b
Pre-emergence action at 320 g/ha against VIOTR in %

| Example number | Dosage [g/ha] | VIOTR |
|---|---|---|
| I-01 | 320 | 90 |
| I-02 | 320 | 100 |
| I-03 | 320 | 100 |
| I-04 | 320 | 100 |
| I-05 | 320 | 100 |
| I-07 | 320 | 100 |
| I-08 | 320 | 100 |
| I-10 | 320 | 100 |
| I-100 | 320 | 90 |
| I-101 | 320 | 100 |
| I-102 | 320 | 100 |
| I-103 | 320 | 90 |
| I-107 | 320 | 100 |
| I-11 | 320 | 100 |
| I-111 | 320 | 100 |
| I-114 | 320 | 90 |
| I-116 | 320 | 90 |
| I-118 | 320 | 100 |
| I-119 | 320 | 90 |
| I-120 | 320 | 80 |
| I-121 | 320 | 100 |
| I-122 | 320 | 100 |
| I-125 | 320 | 90 |
| I-126 | 320 | 100 |
| I-13 | 320 | 90 |
| I-147 | 320 | 90 |
| I-148 | 320 | 100 |
| I-149 | 320 | 100 |
| I-15 | 320 | 100 |
| I-151 | 320 | 100 |
| I-152 | 320 | 90 |
| I-153 | 320 | 100 |
| I-154 | 320 | 100 |
| I-155 | 320 | 100 |
| I-156 | 320 | 90 |
| I-157 | 320 | 100 |
| I-159 | 320 | 100 |
| I-16 | 320 | 100 |
| I-160 | 320 | 100 |
| I-161 | 320 | 90 |
| I-162 | 320 | 100 |
| I-163 | 320 | 100 |
| I-164 | 320 | 100 |
| I-167 | 320 | 100 |
| I-17 | 320 | 100 |
| I-171 | 320 | 100 |
| I-174 | 320 | 100 |
| I-175 | 320 | 100 |
| I-18 | 320 | 100 |
| I-180 | 320 | 100 |
| I-183 | 320 | 100 |
| I-184 | 320 | 100 |
| I-185 | 320 | 100 |
| I-186 | 320 | 90 |
| I-187 | 320 | 100 |
| I-188 | 320 | 100 |
| I-189 | 320 | 100 |
| I-19 | 320 | 100 |
| I-190 | 320 | 100 |
| I-191 | 320 | 100 |
| I-192 | 320 | 100 |
| I-193 | 320 | 100 |
| I-194 | 320 | 100 |
| I-195 | 320 | 100 |
| I-196 | 320 | 100 |
| I-198 | 320 | 100 |
| I-199 | 320 | 100 |
| I-20 | 320 | 100 |
| I-200 | 320 | 100 |
| I-202 | 320 | 100 |
| I-204 | 320 | 100 |
| I-205 | 320 | 100 |
| I-206 | 320 | 100 |
| I-207 | 320 | 100 |
| I-208 | 320 | 100 |
| I-210 | 320 | 100 |
| I-211 | 320 | 100 |
| I-212 | 320 | 100 |
| I-216 | 320 | 100 |
| I-217 | 320 | 90 |
| I-218 | 320 | 100 |
| I-221 | 320 | 90 |
| I-223 | 320 | 100 |
| I-225 | 320 | 100 |
| I-229 | 320 | 90 |
| I-23 | 320 | 100 |
| I-230 | 320 | 90 |
| I-231 | 320 | 100 |
| I-238 | 320 | 90 |
| I-24 | 320 | 100 |
| I-240 | 320 | 90 |
| I-242 | 320 | 100 |
| I-244 | 320 | 90 |
| I-245 | 320 | 80 |
| I-25 | 320 | 100 |
| I-26 | 320 | 100 |
| I-27 | 320 | 100 |
| I-272 | 320 | 90 |
| I-28 | 320 | 100 |
| I-29 | 320 | 100 |
| I-291 | 320 | 100 |
| I-293 | 320 | 100 |
| I-296 | 320 | 100 |
| I-298 | 320 | 100 |
| I-30 | 320 | 100 |
| I-300 | 320 | 90 |
| I-302 | 320 | 90 |
| I-306 | 320 | 80 |
| I-307 | 320 | 100 |
| I-308 | 320 | 100 |
| I-31 | 320 | 90 |
| I-310 | 320 | 100 |
| I-311 | 320 | 100 |
| I-313 | 320 | 90 |
| I-315 | 320 | 100 |
| I-316 | 320 | 100 |
| I-317 | 320 | 100 |
| I-319 | 320 | 100 |
| I-32 | 320 | 100 |
| I-320 | 320 | 100 |
| I-321 | 320 | 100 |
| I-322 | 320 | 100 |
| I-323 | 320 | 100 |
| I-324 | 320 | 100 |
| I-325 | 320 | 100 |
| I-327 | 320 | 100 |
| I-33 | 320 | 100 |
| I-330 | 320 | 100 |
| I-331 | 320 | 100 |
| I-332 | 320 | 100 |
| I-333 | 320 | 100 |
| I-334 | 320 | 90 |
| I-335 | 320 | 100 |
| I-336 | 320 | 100 |
| I-337 | 320 | 100 |
| I-338 | 320 | 100 |
| I-339 | 320 | 100 |
| I-34 | 320 | 100 |
| I-340 | 320 | 100 |
| I-341 | 320 | 100 |
| I-342 | 320 | 100 |
| I-343 | 320 | 100 |
| I-346 | 320 | 80 |
| I-347 | 320 | 100 |
| I-348 | 320 | 90 |
| I-349 | 320 | 80 |
| I-35 | 320 | 100 |
| I-350 | 320 | 100 |
| I-358 | 320 | 100 |
| I-359 | 320 | 100 |
| I-36 | 320 | 100 |
| I-360 | 320 | 100 |
| I-362 | 320 | 100 |
| I-369 | 320 | 100 |
| I-37 | 320 | 100 |
| I-370 | 320 | 100 |
| I-374 | 320 | 100 |
| I-375 | 320 | 100 |
| I-378 | 320 | 90 |
| I-38 | 320 | 100 |
| I-381 | 320 | 90 |
| I-388 | 320 | 100 |
| I-39 | 320 | 100 |
| I-40 | 320 | 100 |
| I-41 | 320 | 100 |
| I-42 | 320 | 100 |
| I-43 | 320 | 100 |
| I-44 | 320 | 100 |
| I-46 | 320 | 100 |
| I-47 | 320 | 100 |
| I-48 | 320 | 100 |
| I-51 | 320 | 100 |
| I-52 | 320 | 100 |
| I-53 | 320 | 100 |
| I-54 | 320 | 100 |
| I-56 | 320 | 100 |
| I-58 | 320 | 80 |
| I-59 | 320 | 90 |
| I-60 | 320 | 80 |
| I-64 | 320 | 90 |
| I-65 | 320 | 100 |
| I-66 | 320 | 100 |
| I-69 | 320 | 90 |
| I-70 | 320 | 90 |
| I-72 | 320 | 90 |
| I-73 | 320 | 100 |
| I-74 | 320 | 100 |
| I-75 | 320 | 100 |
| I-76 | 320 | 100 |
| I-77 | 320 | 100 |
| I-78 | 320 | 80 |
| I-79 | 320 | 100 |
| I-80 | 320 | 90 |
| I-81 | 320 | 100 |
| I-83 | 320 | 100 |
| I-84 | 320 | 100 |
| I-85 | 320 | 100 |
| I-86 | 320 | 100 |
| I-87 | 320 | 80 |
| I-88 | 320 | 90 |
| I-90 | 320 | 90 |
| I-91 | 320 | 100 |
| I-93 | 320 | 100 |
| I-94 | 320 | 100 |
| I-95 | 320 | 100 |
| I-96 | 320 | 90 |
| I-97 | 320 | 100 |
| I-98 | 320 | 100 |
| I-99 | 320 | 90 |
| II-36 | 320 | 100 |
| III-08 | 320 | 90 |
| III-09 | 320 | 80 |
| III-22 | 320 | 100 |
| III-24 | 320 | 100 |

Tables A14 to A18 below show the crop plant compatibilities of selected compounds of the general formula (I) according to Table 1 at an application rate corresponding to 320 g/ha or less, which were observed in trials by the experimental procedure mentioned above. The observed effects on selected crop plants are reported here in comparison to the untreated controls (values in %). The appendices “a” “b” and “c” give differentiation by dosage used with otherwise the same crop plants tested.

TABLE A14a
Pre-emergence action at 20 g/ha against ZEAMX in %

| Example number | Dosage [g/ha] | ZEAMX |
|---|---|---|
| I-01 | 20 | 0 |
| I-02 | 20 | 0 |
| I-11 | 20 | 10 |
| I-13 | 20 | 10 |
| I-16 | 20 | 10 |

TABLE A14b
Pre-emergence action at 80 g/ha against ZEAMX in %

| Example number | Dosage [g/ha] | ZEAMX |
|---|---|---|
| I-01 | 80 | 0 |
| I-04 | 80 | 0 |
| I-05 | 80 | 0 |
| I-10 | 80 | 20 |
| I-103 | 80 | 0 |
| I-108 | 80 | 10 |
| I-109 | 80 | 10 |
| I-11 | 80 | 10 |
| I-110 | 80 | 10 |
| I-111 | 80 | 0 |
| I-112 | 80 | 0 |
| I-114 | 80 | 10 |
| I-122 | 80 | 0 |
| I-125 | 80 | 10 |
| I-147 | 80 | 0 |
| I-152 | 80 | 10 |
| I-156 | 80 | 0 |
| I-157 | 80 | 10 |
| I-161 | 80 | 20 |
| I-19 | 80 | 0 |
| I-200 | 80 | 0 |
| I-201 | 80 | 20 |
| I-202 | 80 | 20 |
| I-223 | 80 | 20 |
| I-225 | 80 | 10 |
| I-229 | 80 | 0 |
| I-23 | 80 | 10 |
| I-235 | 80 | 0 |
| I-236 | 80 | 20 |
| I-237 | 80 | 0 |
| I-239 | 80 | 0 |
| I-24 | 80 | 10 |
| I-241 | 80 | 0 |
| I-242 | 80 | 0 |
| I-243 | 80 | 10 |
| I-244 | 80 | 0 |
| I-245 | 80 | 0 |
| I-246 | 80 | 20 |
| I-247 | 80 | 20 |
| I-25 | 80 | 10 |
| I-26 | 80 | 0 |
| I-27 | 80 | 20 |
| I-272 | 80 | 0 |
| I-278 | 80 | 0 |
| I-28 | 80 | 10 |
| I-293 | 80 | 20 |
| I-297 | 80 | 10 |
| I-30 | 80 | 10 |
| I-300 | 80 | 10 |
| I-302 | 80 | 10 |
| I-305 | 80 | 0 |
| I-306 | 80 | 20 |
| I-309 | 80 | 0 |
| I-31 | 80 | 0 |
| I-310 | 80 | 0 |
| I-312 | 80 | 20 |
| I-315 | 80 | 10 |
| I-325 | 80 | 0 |
| I-327 | 80 | 20 |
| I-329 | 80 | 0 |
| I-330 | 80 | 20 |
| I-334 | 80 | 20 |
| I-335 | 80 | 20 |
| I-345 | 80 | 0 |
| I-346 | 80 | 0 |
| I-349 | 80 | 10 |
| I-354 | 80 | 0 |
| I-357 | 80 | 20 |
| I-359 | 80 | 10 |
| I-36 | 80 | 0 |
| I-362 | 80 | 0 |
| I-363 | 80 | 0 |
| I-371 | 80 | 0 |
| I-373 | 80 | 0 |
| I-376 | 80 | 10 |
| I-377 | 80 | 0 |
| I-378 | 80 | 0 |
| I-381 | 80 | 0 |
| I-386 | 80 | 0 |
| I-47 | 80 | 20 |
| I-49 | 80 | 0 |
| I-50 | 80 | 0 |
| I-53 | 80 | 20 |
| I-54 | 80 | 0 |
| I-55 | 80 | 0 |
| I-58 | 80 | 20 |
| I-60 | 80 | 0 |
| I-62 | 80 | 0 |
| I-63 | 80 | 10 |
| I-70 | 80 | 0 |
| I-71 | 80 | 10 |
| I-72 | 80 | 0 |
| I-78 | 80 | 0 |
| I-88 | 80 | 20 |
| I-90 | 80 | 20 |
| I-93 | 80 | 0 |
| I-95 | 80 | 0 |
| I-96 | 80 | 0 |
| I-99 | 80 | 0 |
| II-36 | 80 | 20 |
| III-01 | 80 | 10 |
| III-02 | 80 | 20 |
| III-07 | 80 | 0 |
| III-09 | 80 | 0 |
| III-17 | 80 | 20 |
| III-18 | 80 | 20 |
| III-20 | 80 | 0 |
| III-21 | 80 | 0 |
| III-23 | 80 | 0 |
| III-25 | 80 | 20 |
| III-26 | 80 | 0 |
| III-27 | 80 | 10 |
| III-28 | 80 | 0 |
| III-30 | 80 | 0 |

TABLE A14c
Pre-emergence action at 320 g/ha against ZEAMX in %

| Example number | Dosage [g/ha] | ZEAMX |
|---|---|---|
| I-108 | 320 | 10 |
| I-109 | 320 | 10 |
| I-112 | 320 | 10 |
| I-114 | 320 | 20 |
| I-156 | 320 | 20 |
| I-161 | 320 | 20 |
| I-200 | 320 | 0 |
| I-239 | 320 | 20 |
| I-309 | 320 | 10 |
| I-325 | 320 | 0 |
| I-329 | 320 | 10 |
| I-345 | 320 | 10 |
| I-346 | 320 | 0 |
| I-349 | 320 | 20 |
| I-363 | 320 | 0 |
| I-371 | 320 | 10 |
| I-373 | 320 | 0 |
| I-377 | 320 | 0 |
| I-386 | 320 | 0 |
| I-49 | 320 | 0 |
| I-50 | 320 | 0 |
| I-55 | 320 | 0 |
| I-63 | 320 | 10 |
| I-70 | 320 | 0 |
| I-72 | 320 | 0 |
| I-78 | 320 | 0 |
| I-95 | 320 | 0 |
| III-01 | 320 | 20 |
| III-07 | 320 | 10 |
| III-09 | 320 | 0 |
| III-17 | 320 | 20 |
| III-18 | 320 | 20 |
| III-20 | 320 | 0 |
| III-21 | 320 | 20 |
| III-23 | 320 | 0 |
| III-26 | 320 | 0 |
| III-27 | 320 | 20 |
| III-28 | 320 | 10 |
| III-30 | 320 | 10 |

TABLE A15a
Pre-emergence action at 20 g/ha against TRZAS in %

| Example number | Dosage [g/ha] | TRZAS |
|---|---|---|
| I-01 | 20 | 0 |
| I-11 | 20 | 0 |
| I-13 | 20 | 10 |
| I-15 | 20 | 10 |
| I-16 | 20 | 10 |
| I-17 | 20 | 10 |

TABLE A15b
Pre-emergence action at 80 g/ha against TRZAS in %

| Example number | Dosage [g/ha] | TRZAS |
|---|---|---|
| I-01 | 80 | 0 |
| I-03 | 80 | 10 |
| I-04 | 80 | 0 |
| I-108 | 80 | 0 |
| I-109 | 80 | 0 |
| I-112 | 80 | 0 |
| I-114 | 80 | 20 |
| I-119 | 80 | 20 |
| I-125 | 80 | 10 |
| I-126 | 80 | 20 |
| I-13 | 80 | 20 |
| I-147 | 80 | 0 |
| I-150 | 80 | 0 |
| I-151 | 80 | 10 |
| I-16 | 80 | 20 |
| I-160 | 80 | 20 |
| I-161 | 80 | 0 |
| I-183 | 80 | 0 |
| I-200 | 80 | 0 |
| I-201 | 80 | 0 |
| I-206 | 80 | 0 |
| I-217 | 80 | 0 |
| I-236 | 80 | 20 |
| I-239 | 80 | 20 |
| I-26 | 80 | 0 |
| I-276 | 80 | 0 |
| I-277 | 80 | 0 |
| I-278 | 80 | 0 |
| I-279 | 80 | 20 |
| I-312 | 80 | 10 |
| I-345 | 80 | 10 |
| I-346 | 80 | 0 |
| I-349 | 80 | 10 |
| I-352 | 80 | 10 |
| I-357 | 80 | 10 |
| I-359 | 80 | 0 |
| I-376 | 80 | 10 |
| I-386 | 80 | 20 |
| I-49 | 80 | 0 |
| I-50 | 80 | 10 |
| I-55 | 80 | 0 |
| I-60 | 80 | 0 |
| I-62 | 80 | 0 |
| I-63 | 80 | 0 |
| I-65 | 80 | 20 |
| I-69 | 80 | 0 |
| I-70 | 80 | 10 |
| I-71 | 80 | 10 |
| I-72 | 80 | 10 |
| I-78 | 80 | 0 |
| I-82 | 80 | 0 |
| III-01 | 80 | 0 |
| III-02 | 80 | 0 |
| III-07 | 80 | 0 |
| III-08 | 80 | 0 |
| III-09 | 80 | 0 |
| III-17 | 80 | 0 |
| III-18 | 80 | 0 |
| III-19 | 80 | 20 |
| III-20 | 80 | 0 |
| III-21 | 80 | 20 |
| III-23 | 80 | 0 |
| III-26 | 80 | 0 |
| III-27 | 80 | 20 |
| III-28 | 80 | 0 |

TABLE A15c
Pre-emergence action at 320 g/ha against TRZAS in %

| Example number | Dosage [g/ha] | TRZAS |
|---|---|---|
| I-108 | 320 | 10 |
| I-109 | 320 | 10 |
| I-112 | 320 | 10 |
| I-147 | 320 | 20 |
| I-150 | 320 | 0 |
| I-161 | 320 | 20 |
| I-200 | 320 | 10 |
| I-276 | 320 | 0 |
| I-312 | 320 | 20 |
| I-357 | 320 | 10 |
| I-49 | 320 | 0 |
| I-50 | 320 | 10 |
| I-55 | 320 | 0 |
| I-62 | 320 | 0 |
| I-63 | 320 | 0 |
| I-82 | 320 | 20 |
| III-02 | 320 | 20 |
| III-07 | 320 | 0 |
| III-08 | 320 | 0 |
| III-09 | 320 | 0 |
| III-18 | 320 | 20 |
| III-20 | 320 | 0 |
| III-26 | 320 | 20 |

TABLE A16a
Pre-emergence action at 20 g/ha against ORYSA in %

| Example number | Dosage [g/ha] | ORYSA |
|---|---|---|
| I-02 | 20 | 0 |
| I-11 | 20 | 0 |
| I-13 | 20 | 0 |
| I-16 | 20 | 20 |
| I-17 | 20 | 0 |

TABLE A16b
Pre-emergence action at 80 g/ha against ORYSA in %

| Example number | Dosage [g/ha] | ORYSA |
|---|---|---|
| I-103 | 80 | 0 |
| I-107 | 80 | 10 |
| I-108 | 80 | 0 |
| I-109 | 80 | 10 |
| I-11 | 80 | 20 |
| I-110 | 80 | 10 |
| I-111 | 80 | 0 |
| I-112 | 80 | 0 |
| I-114 | 80 | 0 |
| I-119 | 80 | 10 |
| I-120 | 80 | 0 |
| I-121 | 80 | 0 |
| I-122 | 80 | 0 |
| I-125 | 80 | 10 |
| I-126 | 80 | 20 |
| I-13 | 80 | 10 |
| I-150 | 80 | 20 |
| I-152 | 80 | 20 |
| I-156 | 80 | 0 |
| I-161 | 80 | 0 |
| I-193 | 80 | 20 |
| I-200 | 80 | 20 |
| I-201 | 80 | 0 |
| I-223 | 80 | 10 |
| I-225 | 80 | 20 |
| I-23 | 80 | 10 |
| I-246 | 80 | 0 |
| I-26 | 80 | 0 |
| I-272 | 80 | 10 |
| I-278 | 80 | 0 |
| I-279 | 80 | 0 |
| I-28 | 80 | 0 |
| I-297 | 80 | 10 |
| I-302 | 80 | 20 |
| I-31 | 80 | 0 |
| I-310 | 80 | 0 |
| I-312 | 80 | 20 |
| I-329 | 80 | 0 |
| I-335 | 80 | 0 |
| I-336 | 80 | 20 |
| I-337 | 80 | 20 |
| I-345 | 80 | 10 |
| I-35 | 80 | 20 |
| I-351 | 80 | 20 |
| I-352 | 80 | 20 |
| I-357 | 80 | 20 |
| I-360 | 80 | 10 |
| I-362 | 80 | 20 |
| I-369 | 80 | 20 |
| I-371 | 80 | 0 |
| I-376 | 80 | 20 |
| I-378 | 80 | 0 |
| I-381 | 80 | 20 |
| I-386 | 80 | 0 |
| I-49 | 80 | 10 |
| I-55 | 80 | 0 |
| I-58 | 80 | 0 |
| I-59 | 80 | 20 |
| I-60 | 80 | 0 |
| I-62 | 80 | 0 |
| I-63 | 80 | 0 |
| I-64 | 80 | 20 |
| I-69 | 80 | 0 |
| I-70 | 80 | 0 |
| I-71 | 80 | 10 |
| I-72 | 80 | 0 |
| I-78 | 80 | 0 |
| I-82 | 80 | 0 |
| I-90 | 80 | 10 |
| I-95 | 80 | 0 |
| I-96 | 80 | 20 |
| I-97 | 80 | 10 |
| I-99 | 80 | 0 |
| II-36 | 80 | 0 |
| III-01 | 80 | 0 |
| III-02 | 80 | 0 |
| III-07 | 80 | 0 |
| III-08 | 80 | 0 |
| III-09 | 80 | 0 |
| III-17 | 80 | 0 |
| III-18 | 80 | 0 |
| III-19 | 80 | 0 |
| III-20 | 80 | 0 |
| III-21 | 80 | 0 |
| III-23 | 80 | 0 |
| III-25 | 80 | 0 |
| III-26 | 80 | 0 |
| III-27 | 80 | 0 |
| III-28 | 80 | 0 |
| III-30 | 80 | 10 |

TABLE A16c
Pre-emergence action at 320 g/ha against ORYSA in %

| Example number | Dosage [g/ha] | ORYSA |
|---|---|---|
| I-108 | 320 | 10 |
| I-109 | 320 | 10 |
| I-112 | 320 | 10 |
| I-114 | 320 | 0 |
| I-120 | 320 | 10 |
| I-161 | 320 | 0 |
| I-200 | 320 | 20 |
| I-201 | 320 | 20 |
| I-297 | 320 | 20 |
| I-31 | 320 | 0 |
| I-329 | 320 | 20 |
| I-345 | 320 | 20 |
| I-346 | 320 | 0 |
| I-357 | 320 | 20 |
| I-371 | 320 | 0 |
| I-386 | 320 | 20 |
| I-49 | 320 | 20 |
| I-55 | 320 | 0 |
| I-62 | 320 | 0 |
| I-63 | 320 | 0 |
| I-70 | 320 | 10 |
| I-72 | 320 | 20 |
| I-78 | 320 | 0 |
| I-95 | 320 | 0 |
| III-01 | 320 | 0 |
| III-02 | 320 | 0 |
| III-07 | 320 | 20 |
| III-08 | 320 | 0 |
| III-09 | 320 | 0 |
| III-17 | 320 | 10 |
| III-18 | 320 | 0 |
| III-19 | 320 | 20 |
| III-20 | 320 | 10 |
| III-21 | 320 | 0 |
| III-25 | 320 | 0 |
| III-26 | 320 | 0 |
| III-27 | 320 | 20 |
| III-28 | 320 | 10 |
| III-30 | 320 | 20 |

TABLE A17a
Pre-emergence action at 20 g/ha against GLXMA in %

| Example number | Dosage [g/ha] | GLXMA |
|---|---|---|
| I-01 | 20 | 0 |
| I-11 | 20 | 10 |
| I-13 | 20 | 10 |
| I-17 | 20 | 10 |

TABLE A17b
Pre-emergence action at 80 g/ha against GLXMA in %

| Example number | Dosage [g/ha] | GLXMA |
|---|---|---|
| I-01 | 80 | 20 |
| I-108 | 80 | 0 |
| I-109 | 80 | 20 |
| I-13 | 80 | 10 |
| I-147 | 80 | 0 |
| I-150 | 80 | 0 |
| I-151 | 80 | 10 |
| I-161 | 80 | 0 |
| I-183 | 80 | 0 |
| I-200 | 80 | 20 |
| I-201 | 80 | 0 |
| I-206 | 80 | 10 |
| I-230 | 80 | 20 |
| I-238 | 80 | 20 |
| I-240 | 80 | 20 |
| I-242 | 80 | 0 |
| I-244 | 80 | 0 |
| I-298 | 80 | 20 |
| I-306 | 80 | 0 |
| I-309 | 80 | 0 |
| I-312 | 80 | 20 |
| I-329 | 80 | 0 |
| I-345 | 80 | 0 |
| I-346 | 80 | 0 |
| I-347 | 80 | 10 |
| I-349 | 80 | 0 |
| I-363 | 80 | 0 |
| I-371 | 80 | 20 |
| I-386 | 80 | 0 |
| I-55 | 80 | 0 |
| I-58 | 80 | 0 |
| I-60 | 80 | 20 |
| I-65 | 80 | 20 |
| I-70 | 80 | 10 |
| I-71 | 80 | 20 |
| I-72 | 80 | 0 |
| I-77 | 80 | 0 |
| I-78 | 80 | 0 |
| I-82 | 80 | 0 |
| II-36 | 80 | 0 |
| III-09 | 80 | 20 |
| III-17 | 80 | 0 |
| III-18 | 80 | 0 |
| III-19 | 80 | 0 |
| III-21 | 80 | 0 |
| III-23 | 80 | 0 |
| III-25 | 80 | 0 |
| III-26 | 80 | 0 |
| III-27 | 80 | 0 |
| III-30 | 80 | 20 |

TABLE A17c
Pre-emergence action at 320 g/ha against GLXMA in %

| Example number | Dosage [g/ha] | GLXMA |
|---|---|---|
| I-69 | 320 | 20 |
| I-55 | 320 | 0 |
| I-108 | 320 | 0 |
| I-109 | 320 | 20 |
| I-112 | 320 | 20 |
| III-21 | 320 | 0 |
| III-20 | 320 | 20 |
| I-200 | 320 | 20 |
| I-151 | 320 | 20 |
| I-147 | 320 | 0 |
| I-150 | 320 | 0 |
| III-30 | 320 | 20 |
| I-386 | 320 | 0 |
| I-346 | 320 | 20 |
| I-363 | 320 | 0 |
| I-349 | 320 | 10 |

TABLE A18a
Pre-emergence action at 20 g/ha against BRSNW in %

| Example number | Dosage [g/ha] | BRSNW |
|---|---|---|
| I-01 | 20 | 0 |
| I-11 | 20 | 10 |
| I-13 | 20 | 10 |
| I-15 | 20 | 10 |
| I-16 | 20 | 10 |

TABLE A18b
Pre-emergence action at 80 g/ha against BRSNW in %

| Example number | Dosage [g/ha] | BRSNW |
|---|---|---|
| I-101 | 80 | 10 |
| I-108 | 80 | 10 |
| I-109 | 80 | 10 |
| I-11 | 80 | 20 |
| I-110 | 80 | 20 |
| I-112 | 80 | 10 |
| I-125 | 80 | 20 |
| I-13 | 80 | 10 |
| I-147 | 80 | 0 |
| I-150 | 80 | 0 |
| I-161 | 80 | 10 |
| I-200 | 80 | 0 |
| I-24 | 80 | 20 |
| I-242 | 80 | 20 |
| I-245 | 80 | 0 |
| I-25 | 80 | 10 |
| I-26 | 80 | 20 |
| I-272 | 80 | 0 |
| I-297 | 80 | 20 |
| I-300 | 80 | 20 |
| I-312 | 80 | 20 |
| I-319 | 80 | 20 |
| I-325 | 80 | 20 |
| I-329 | 80 | 0 |
| I-335 | 80 | 0 |
| I-345 | 80 | 20 |
| I-346 | 80 | 0 |
| I-351 | 80 | 20 |
| I-363 | 80 | 0 |
| I-371 | 80 | 0 |
| I-377 | 80 | 0 |
| I-378 | 80 | 0 |
| I-381 | 80 | 0 |
| I-386 | 80 | 0 |
| I-49 | 80 | 10 |
| I-50 | 80 | 0 |
| I-55 | 80 | 0 |
| I-58 | 80 | 0 |
| I-62 | 80 | 0 |
| I-63 | 80 | 0 |
| I-65 | 80 | 20 |
| I-69 | 80 | 20 |
| I-70 | 80 | 20 |
| I-71 | 80 | 0 |
| I-72 | 80 | 10 |
| I-82 | 80 | 20 |
| II-36 | 80 | 0 |
| III-01 | 80 | 0 |
| III-07 | 80 | 0 |
| III-20 | 80 | 0 |
| III-21 | 80 | 20 |
| III-23 | 80 | 0 |
| III-26 | 80 | 0 |

TABLE A18c
Pre-emergence action at 320 g/ha against BRSNW in %

| Example number | Dosage [g/ha] | BRSNW |
|---|---|---|
| I-108 | 320 | 20 |
| I-109 | 320 | 10 |
| I-112 | 320 | 10 |
| I-13 | 320 | 20 |
| I-161 | 320 | 10 |
| I-200 | 320 | 0 |
| I-335 | 320 | 0 |
| I-346 | 320 | 20 |
| I-363 | 320 | 20 |
| I-371 | 320 | 20 |
| I-386 | 320 | 0 |
| I-50 | 320 | 20 |
| I-55 | 320 | 0 |
| I-63 | 320 | 20 |
| I-82 | 320 | 20 |
| III-21 | 320 | 20 |
| III-23 | 320 | 0 |

As the results from Tables A1 to A18 show, inventive compounds of the general formula (I) in the case of pre-emergence treatment have good herbicidal efficacy against harmful plants, for example Abutilon theophrasti (ABUTH), Alopecurus myosuroides (ALOMY), Amaranthus retroflexus (AMARE), Avena fatua (AVEFA), Digitaria sanguinalis (DIGSA), Echinochloa crus-galli (ECHCG), Lolium rigidum (LOLRI), Matricaria inodora (MATIN), Pharbitis purpurea (PHBPU), Polygonum convolvulus (POLCO), Setaria viridis (SETVI), Verconica persica (VERPE) and Viola tricolor (VIOTR) at an application rate of 320 g of active substance or less per hectare, and good crop plant compatibility in the case of organisms such as Oryza sativa (ORYSA), Zea mays (ZEAMX), Brassica napus (BRSNW), Glycine max (GLXMA) and Triticum aestivum (TRZAS) at an application rate of 320 g or less per hectare.

The compounds of the invention are therefore suitable for control of unwanted plant growth by the pre-emergence method.

B. Post-Emergence Herbicidal Action and Crop Plant Compatibility

Seeds of monocotyledonous and dicotyledonous weeds and crop plants are placed in sandy loam in plastic or organic planting pots, covered with soil and cultivated in a greenhouse under controlled growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion with addition of 0.5% additive at a water application rate of 600 l/ha (converted). After the test plants have been kept in the greenhouse under optimum growth conditions for about 3 weeks, the activity of the preparations is rated visually in comparison to untreated controls. For example, 100% activity=the plants have died, 0% activity=like control plants.

Tables B1 to B13 below show the effects of selected compounds of the general formula (I) according to Table 1 on various harmful plants and at an application rate corresponding to 320 g/ha or lower, which were obtained by the experimental procedure mentioned above. The appendices “a”, “b” and “c” give differentiation by dosage used with otherwise the same harmful plants tested.

TABLE B1a
Post-emergence action at 80 g/ha against ABUTH in %

| Example number | Dosage [g/ha] | ABUTH |
|---|---|---|
| I-02 | 80 | 80 |
| I-03 | 80 | 80 |
| I-04 | 80 | 90 |
| I-05 | 80 | 80 |
| I-07 | 80 | 80 |
| I-08 | 80 | 80 |
| I-100 | 80 | 80 |
| I-102 | 80 | 80 |
| I-103 | 80 | 80 |
| I-118 | 80 | 80 |
| I-119 | 80 | 80 |
| I-120 | 80 | 80 |
| I-125 | 80 | 80 |
| I-126 | 80 | 80 |
| I-130 | 80 | 80 |
| I-144 | 80 | 80 |
| I-145 | 80 | 90 |
| I-146 | 80 | 80 |
| I-151 | 80 | 80 |
| I-165 | 80 | 90 |
| I-167 | 80 | 80 |
| I-168 | 80 | 80 |
| I-174 | 80 | 80 |
| I-175 | 80 | 80 |
| I-177 | 80 | 90 |
| I-179 | 80 | 80 |
| I-181 | 80 | 80 |
| I-182 | 80 | 80 |
| I-183 | 80 | 80 |
| I-184 | 80 | 80 |
| I-185 | 80 | 80 |
| I-188 | 80 | 80 |
| I-19 | 80 | 80 |
| I-190 | 80 | 80 |
| I-191 | 80 | 80 |
| I-193 | 80 | 80 |
| I-196 | 80 | 80 |
| I-197 | 80 | 90 |
| I-198 | 80 | 80 |
| I-208 | 80 | 80 |
| I-212 | 80 | 80 |
| I-217 | 80 | 80 |
| I-218 | 80 | 80 |
| I-227 | 80 | 90 |
| I-267 | 80 | 80 |
| I-275 | 80 | 80 |
| I-28 | 80 | 80 |
| I-304 | 80 | 80 |
| I-314 | 80 | 80 |
| I-315 | 80 | 80 |
| I-318 | 80 | 80 |
| I-325 | 80 | 80 |
| I-326 | 80 | 80 |
| I-327 | 80 | 80 |
| I-328 | 80 | 80 |
| I-330 | 80 | 80 |
| I-331 | 80 | 90 |
| I-332 | 80 | 80 |
| I-334 | 80 | 80 |
| I-337 | 80 | 80 |
| I-339 | 80 | 80 |
| I-341 | 80 | 80 |
| I-342 | 80 | 80 |
| I-343 | 80 | 80 |
| I-344 | 80 | 80 |
| I-35 | 80 | 90 |
| I-36 | 80 | 80 |
| I-366 | 80 | 80 |
| I-37 | 80 | 80 |
| I-374 | 80 | 80 |
| I-375 | 80 | 90 |
| I-38 | 80 | 80 |
| I-380 | 80 | 80 |
| I-39 | 80 | 90 |
| I-42 | 80 | 90 |
| I-45 | 80 | 80 |
| I-48 | 80 | 90 |
| I-54 | 80 | 80 |
| I-56 | 80 | 80 |
| I-59 | 80 | 80 |
| I-64 | 80 | 90 |
| I-73 | 80 | 80 |
| I-74 | 80 | 80 |
| I-75 | 80 | 80 |
| I-76 | 80 | 90 |
| I-77 | 80 | 80 |
| I-78 | 80 | 80 |
| I-79 | 80 | 80 |
| I-81 | 80 | 80 |
| I-83 | 80 | 90 |
| I-84 | 80 | 80 |
| I-85 | 80 | 90 |
| I-86 | 80 | 90 |
| I-88 | 80 | 80 |
| I-95 | 80 | 80 |
| I-96 | 80 | 90 |
| I-97 | 80 | 90 |
| III-22 | 80 | 90 |
| III-24 | 80 | 90 |

TABLE B1b
Post-emergence action at 320 g/ha against ABUTH in %

| Example number | Dosage [g/ha] | ABUTH |
|---|---|---|
| I-01 | 320 | 80 |
| I-02 | 320 | 80 |
| I-03 | 320 | 90 |
| I-04 | 320 | 90 |
| I-05 | 320 | 80 |
| I-07 | 320 | 90 |
| I-08 | 320 | 90 |
| I-10 | 320 | 80 |
| I-130 | 320 | 90 |
| I-133 | 320 | 80 |
| I-137 | 320 | 80 |
| I-138 | 320 | 80 |
| I-139 | 320 | 80 |
| I-140 | 320 | 80 |
| I-144 | 320 | 90 |
| I-145 | 320 | 90 |
| I-146 | 320 | 90 |
| I-151 | 320 | 80 |
| I-152 | 320 | 80 |
| I-157 | 320 | 80 |
| I-159 | 320 | 80 |
| I-165 | 320 | 90 |
| I-167 | 320 | 80 |
| I-168 | 320 | 80 |
| I-171 | 320 | 90 |
| I-172 | 320 | 80 |
| I-174 | 320 | 80 |
| I-175 | 320 | 80 |
| I-176 | 320 | 80 |
| I-177 | 320 | 90 |
| I-179 | 320 | 80 |
| I-18 | 320 | 80 |
| I-181 | 320 | 80 |
| I-182 | 320 | 80 |
| I-183 | 320 | 80 |
| I-184 | 320 | 90 |
| I-185 | 320 | 80 |
| I-186 | 320 | 80 |
| I-187 | 320 | 80 |
| I-188 | 320 | 80 |
| I-190 | 320 | 80 |
| I-191 | 320 | 80 |
| I-192 | 320 | 80 |
| I-193 | 320 | 80 |
| I-194 | 320 | 80 |
| I-195 | 320 | 90 |
| I-196 | 320 | 80 |
| I-197 | 320 | 90 |
| I-198 | 320 | 80 |
| I-199 | 320 | 80 |
| I-202 | 320 | 80 |
| I-207 | 320 | 80 |
| I-208 | 320 | 90 |
| I-210 | 320 | 80 |
| I-212 | 320 | 80 |
| I-216 | 320 | 80 |
| I-217 | 320 | 90 |
| I-218 | 320 | 80 |
| I-223 | 320 | 80 |
| I-225 | 320 | 80 |
| I-227 | 320 | 90 |
| I-249 | 320 | 80 |
| I-26 | 320 | 90 |
| I-260 | 320 | 80 |
| I-261 | 320 | 80 |
| I-267 | 320 | 90 |
| I-268 | 320 | 80 |
| I-27 | 320 | 90 |
| I-273 | 320 | 80 |
| I-274 | 320 | 80 |
| I-275 | 320 | 80 |
| I-28 | 320 | 90 |
| I-285 | 320 | 80 |
| I-287 | 320 | 90 |
| I-289 | 320 | 80 |
| I-291 | 320 | 80 |
| I-294 | 320 | 80 |
| I-296 | 320 | 80 |
| I-304 | 320 | 90 |
| I-307 | 320 | 90 |
| I-309 | 320 | 80 |
| I-311 | 320 | 80 |
| I-312 | 320 | 80 |
| I-314 | 320 | 80 |
| I-315 | 320 | 80 |
| I-318 | 320 | 80 |
| I-319 | 320 | 80 |
| I-320 | 320 | 80 |
| I-325 | 320 | 90 |
| I-326 | 320 | 80 |
| I-327 | 320 | 90 |
| I-328 | 320 | 80 |
| I-330 | 320 | 80 |
| I-331 | 320 | 90 |
| I-332 | 320 | 80 |
| I-333 | 320 | 80 |
| I-334 | 320 | 80 |
| I-336 | 320 | 80 |
| I-337 | 320 | 80 |
| I-339 | 320 | 90 |
| I-340 | 320 | 80 |
| I-341 | 320 | 90 |
| I-342 | 320 | 90 |
| I-343 | 320 | 90 |
| I-344 | 320 | 80 |
| I-348 | 320 | 80 |
| I-350 | 320 | 90 |
| I-358 | 320 | 80 |
| I-359 | 320 | 80 |
| I-360 | 320 | 80 |
| I-366 | 320 | 90 |
| I-369 | 320 | 80 |
| I-373 | 320 | 80 |
| I-375 | 320 | 90 |
| I-376 | 320 | 80 |
| I-378 | 320 | 80 |
| I-380 | 320 | 90 |
| I-388 | 320 | 80 |
| I-41 | 320 | 80 |
| I-42 | 320 | 90 |
| I-48 | 320 | 90 |
| I-66 | 320 | 80 |
| I-69 | 320 | 80 |
| I-85 | 320 | 90 |
| I-86 | 320 | 90 |
| I-88 | 320 | 80 |
| I-94 | 320 | 80 |
| I-95 | 320 | 80 |
| I-96 | 320 | 90 |
| II-38 | 320 | 80 |
| II-39 | 320 | 80 |
| II-40 | 320 | 80 |
| II-41 | 320 | 80 |
| II-43 | 320 | 80 |
| II-44 | 320 | 80 |
| II-45 | 320 | 80 |
| III-07 | 320 | 80 |
| III-22 | 320 | 90 |
| III-23 | 320 | 80 |
| III-24 | 320 | 90 |
| III-29 | 320 | 80 |

TABLE B2a
Post-emergence action at 80 g/ha against ALOMY in %

| Example number | Dosage [g/ha] | ALOMY |
|---|---|---|
| I-01 | 80 | 100 |
| I-02 | 80 | 80 |
| I-05 | 80 | 90 |
| I-07 | 80 | 90 |
| I-08 | 80 | 100 |
| I-10 | 80 | 100 |
| I-100 | 80 | 100 |
| I-101 | 80 | 90 |
| I-102 | 80 | 90 |
| I-107 | 80 | 90 |
| I-11 | 80 | 90 |
| I-110 | 80 | 80 |
| I-114 | 80 | 90 |
| I-116 | 80 | 90 |
| I-118 | 80 | 100 |
| I-119 | 80 | 90 |
| I-120 | 80 | 100 |
| I-121 | 80 | 80 |
| I-125 | 80 | 90 |
| I-13 | 80 | 90 |
| I-139 | 80 | 90 |
| I-144 | 80 | 90 |
| I-145 | 80 | 80 |
| I-148 | 80 | 80 |
| I-149 | 80 | 80 |
| I-15 | 80 | 90 |
| I-151 | 80 | 80 |
| I-16 | 80 | 90 |
| I-164 | 80 | 100 |
| I-166 | 80 | 90 |
| I-167 | 80 | 90 |
| I-17 | 80 | 90 |
| I-171 | 80 | 90 |
| I-172 | 80 | 90 |
| I-174 | 80 | 90 |
| I-175 | 80 | 90 |
| I-18 | 80 | 90 |
| I-180 | 80 | 90 |
| I-181 | 80 | 80 |
| I-184 | 80 | 80 |
| I-188 | 80 | 80 |
| I-189 | 80 | 80 |
| I-19 | 80 | 100 |
| I-190 | 80 | 80 |
| I-191 | 80 | 80 |
| I-192 | 80 | 90 |
| I-193 | 80 | 90 |
| I-194 | 80 | 90 |
| I-195 | 80 | 80 |
| I-196 | 80 | 80 |
| I-197 | 80 | 90 |
| I-198 | 80 | 80 |
| I-20 | 80 | 100 |
| I-204 | 80 | 90 |
| I-211 | 80 | 80 |
| I-212 | 80 | 90 |
| I-221 | 80 | 80 |
| I-227 | 80 | 80 |
| I-23 | 80 | 90 |
| I-24 | 80 | 90 |
| I-25 | 80 | 90 |
| I-252 | 80 | 80 |
| I-27 | 80 | 80 |
| I-275 | 80 | 80 |
| I-28 | 80 | 100 |
| I-285 | 80 | 90 |
| I-289 | 80 | 80 |
| I-29 | 80 | 100 |
| I-291 | 80 | 80 |
| I-293 | 80 | 90 |
| I-296 | 80 | 80 |
| I-299 | 80 | 80 |
| I-30 | 80 | 90 |
| I-300 | 80 | 80 |
| I-302 | 80 | 80 |
| I-307 | 80 | 100 |
| I-308 | 80 | 100 |
| I-311 | 80 | 80 |
| I-313 | 80 | 90 |
| I-314 | 80 | 90 |
| I-315 | 80 | 80 |
| I-316 | 80 | 100 |
| I-317 | 80 | 100 |
| I-318 | 80 | 90 |
| I-319 | 80 | 90 |
| I-32 | 80 | 90 |
| I-321 | 80 | 90 |
| I-323 | 80 | 100 |
| I-325 | 80 | 80 |
| I-326 | 80 | 90 |
| I-328 | 80 | 90 |
| I-33 | 80 | 90 |
| I-330 | 80 | 80 |
| I-331 | 80 | 100 |
| I-332 | 80 | 80 |
| I-333 | 80 | 90 |
| I-336 | 80 | 90 |
| I-337 | 80 | 90 |
| I-339 | 80 | 90 |
| I-34 | 80 | 90 |
| I-340 | 80 | 90 |
| I-341 | 80 | 80 |
| I-342 | 80 | 100 |
| I-343 | 80 | 90 |
| I-344 | 80 | 90 |
| I-348 | 80 | 80 |
| I-35 | 80 | 90 |
| I-358 | 80 | 90 |
| I-36 | 80 | 90 |
| I-360 | 80 | 90 |
| I-369 | 80 | 80 |
| I-37 | 80 | 90 |
| I-370 | 80 | 100 |
| I-374 | 80 | 100 |
| I-375 | 80 | 100 |
| I-38 | 80 | 100 |
| I-39 | 80 | 100 |
| I-40 | 80 | 100 |
| I-41 | 80 | 90 |
| I-42 | 80 | 100 |
| I-43 | 80 | 90 |
| I-44 | 80 | 80 |
| I-45 | 80 | 90 |
| I-46 | 80 | 90 |
| I-48 | 80 | 100 |
| I-51 | 80 | 90 |
| I-52 | 80 | 90 |
| I-53 | 80 | 90 |
| I-54 | 80 | 90 |
| I-56 | 80 | 90 |
| I-59 | 80 | 90 |
| I-64 | 80 | 100 |
| I-66 | 80 | 100 |
| I-71 | 80 | 90 |
| I-72 | 80 | 90 |
| I-73 | 80 | 100 |
| I-74 | 80 | 100 |
| I-75 | 80 | 90 |
| I-76 | 80 | 100 |
| I-77 | 80 | 90 |
| I-79 | 80 | 80 |
| I-81 | 80 | 90 |
| I-83 | 80 | 100 |
| I-84 | 80 | 80 |
| I-86 | 80 | 100 |
| I-87 | 80 | 80 |
| I-88 | 80 | 80 |
| I-90 | 80 | 90 |
| I-91 | 80 | 90 |
| I-93 | 80 | 100 |
| I-94 | 80 | 80 |
| I-96 | 80 | 90 |
| I-97 | 80 | 90 |
| I-98 | 80 | 90 |
| I-99 | 80 | 90 |
| II-42 | 80 | 80 |
| III-22 | 80 | 100 |
| III-24 | 80 | 100 |

TABLE B2b
Post-emergence action at 320 g/ha against ALOMY in %

| Example number | Dosage [g/ha] | ALOMY |
|---|---|---|
| I-01 | 320 | 100 |
| I-02 | 320 | 100 |
| I-03 | 320 | 100 |
| I-05 | 320 | 100 |
| I-07 | 320 | 100 |
| I-08 | 320 | 100 |
| I-10 | 320 | 100 |
| I-11 | 320 | 90 |
| I-111 | 320 | 100 |
| I-13 | 320 | 90 |
| I-130 | 320 | 90 |
| I-133 | 320 | 90 |
| I-134 | 320 | 90 |
| I-136 | 320 | 90 |
| I-137 | 320 | 80 |
| I-139 | 320 | 90 |
| I-140 | 320 | 100 |
| I-141 | 320 | 90 |
| I-144 | 320 | 90 |
| I-145 | 320 | 90 |
| I-146 | 320 | 90 |
| I-148 | 320 | 90 |
| I-149 | 320 | 90 |
| I-15 | 320 | 90 |
| I-151 | 320 | 80 |
| I-152 | 320 | 80 |
| I-153 | 320 | 80 |
| I-155 | 320 | 80 |
| I-156 | 320 | 80 |
| I-16 | 320 | 90 |
| I-163 | 320 | 80 |
| I-164 | 320 | 100 |
| I-165 | 320 | 100 |
| I-166 | 320 | 90 |
| I-167 | 320 | 90 |
| I-17 | 320 | 90 |
| I-171 | 320 | 90 |
| I-172 | 320 | 90 |
| I-174 | 320 | 90 |
| I-175 | 320 | 90 |
| I-176 | 320 | 90 |
| I-177 | 320 | 100 |
| I-179 | 320 | 90 |
| I-18 | 320 | 100 |
| I-180 | 320 | 90 |
| I-181 | 320 | 90 |
| I-182 | 320 | 80 |
| I-184 | 320 | 90 |
| I-185 | 320 | 80 |
| I-187 | 320 | 90 |
| I-188 | 320 | 90 |
| I-189 | 320 | 90 |
| I-190 | 320 | 100 |
| I-191 | 320 | 90 |
| I-192 | 320 | 90 |
| I-193 | 320 | 100 |
| I-194 | 320 | 90 |
| I-195 | 320 | 90 |
| I-196 | 320 | 90 |
| I-197 | 320 | 90 |
| I-198 | 320 | 80 |
| I-199 | 320 | 90 |
| I-20 | 320 | 100 |
| I-204 | 320 | 90 |
| I-206 | 320 | 80 |
| I-207 | 320 | 90 |
| I-208 | 320 | 80 |
| I-210 | 320 | 90 |
| I-211 | 320 | 90 |
| I-212 | 320 | 90 |
| I-218 | 320 | 90 |
| I-221 | 320 | 80 |
| I-223 | 320 | 80 |
| I-225 | 320 | 90 |
| I-227 | 320 | 90 |
| I-237 | 320 | 80 |
| I-238 | 320 | 80 |
| I-240 | 320 | 80 |
| I-242 | 320 | 80 |
| I-244 | 320 | 80 |
| I-245 | 320 | 80 |
| I-247 | 320 | 80 |
| I-251 | 320 | 80 |
| I-252 | 320 | 90 |
| I-26 | 320 | 90 |
| I-260 | 320 | 90 |
| I-264 | 320 | 90 |
| I-266 | 320 | 90 |
| I-267 | 320 | 90 |
| I-27 | 320 | 100 |
| I-272 | 320 | 90 |
| I-274 | 320 | 90 |
| I-275 | 320 | 100 |
| I-28 | 320 | 100 |
| I-285 | 320 | 90 |
| I-287 | 320 | 90 |
| I-288 | 320 | 90 |
| I-289 | 320 | 90 |
| I-290 | 320 | 90 |
| I-291 | 320 | 80 |
| I-293 | 320 | 90 |
| I-294 | 320 | 90 |
| I-296 | 320 | 90 |
| I-298 | 320 | 90 |
| I-299 | 320 | 90 |
| I-300 | 320 | 90 |
| I-302 | 320 | 90 |
| I-304 | 320 | 90 |
| I-305 | 320 | 100 |
| I-306 | 320 | 100 |
| I-307 | 320 | 100 |
| I-308 | 320 | 100 |
| I-310 | 320 | 90 |
| I-311 | 320 | 90 |
| I-312 | 320 | 90 |
| I-313 | 320 | 90 |
| I-314 | 320 | 90 |
| I-315 | 320 | 90 |
| I-316 | 320 | 100 |
| I-317 | 320 | 100 |
| I-318 | 320 | 90 |
| I-319 | 320 | 90 |
| I-320 | 320 | 100 |
| I-321 | 320 | 100 |
| I-322 | 320 | 80 |
| I-323 | 320 | 100 |
| I-324 | 320 | 100 |
| I-325 | 320 | 100 |
| I-326 | 320 | 90 |
| I-327 | 320 | 100 |
| I-328 | 320 | 90 |
| I-330 | 320 | 90 |
| I-331 | 320 | 100 |
| I-332 | 320 | 90 |
| I-333 | 320 | 90 |
| I-334 | 320 | 90 |
| I-335 | 320 | 100 |
| I-336 | 320 | 90 |
| I-337 | 320 | 90 |
| I-338 | 320 | 90 |
| I-339 | 320 | 100 |
| I-340 | 320 | 100 |
| I-341 | 320 | 100 |
| I-342 | 320 | 100 |
| I-343 | 320 | 100 |
| I-344 | 320 | 90 |
| I-347 | 320 | 80 |
| I-348 | 320 | 90 |
| I-350 | 320 | 90 |
| I-354 | 320 | 90 |
| I-358 | 320 | 90 |
| I-359 | 320 | 90 |
| I-360 | 320 | 90 |
| I-362 | 320 | 90 |
| I-366 | 320 | 90 |
| I-369 | 320 | 100 |
| I-370 | 320 | 100 |
| I-375 | 320 | 100 |
| I-376 | 320 | 80 |
| I-380 | 320 | 90 |
| I-382 | 320 | 90 |
| I-383 | 320 | 90 |
| I-388 | 320 | 80 |
| I-41 | 320 | 90 |
| I-42 | 320 | 100 |
| I-48 | 320 | 100 |
| I-58 | 320 | 90 |
| I-66 | 320 | 100 |
| I-69 | 320 | 90 |
| I-80 | 320 | 90 |
| I-85 | 320 | 100 |
| I-86 | 320 | 100 |
| I-87 | 320 | 90 |
| I-88 | 320 | 90 |
| I-94 | 320 | 100 |
| I-95 | 320 | 100 |
| I-96 | 320 | 90 |
| II-38 | 320 | 100 |
| II-39 | 320 | 90 |
| II-40 | 320 | 90 |
| II-41 | 320 | 90 |
| II-42 | 320 | 90 |
| II-43 | 320 | 100 |
| II-44 | 320 | 100 |
| II-45 | 320 | 90 |
| II-46 | 320 | 80 |
| III-08 | 320 | 80 |
| III-22 | 320 | 100 |
| III-23 | 320 | 100 |
| III-24 | 320 | 100 |
| III-29 | 320 | 90 |
| III-30 | 320 | 80 |

TABLE B3a
Post-emergence action at 80 g/ha against AMARE in %

| Example number | Dosage [g/ha] | AMARE |
|---|---|---|
| I-02 | 80 | 80 |
| I-03 | 80 | 80 |
| I-04 | 80 | 90 |
| I-05 | 80 | 90 |
| I-07 | 80 | 90 |
| I-08 | 80 | 80 |
| I-100 | 80 | 90 |
| I-102 | 80 | 80 |
| I-103 | 80 | 80 |
| I-107 | 80 | 80 |
| I-111 | 80 | 80 |
| I-116 | 80 | 90 |
| I-118 | 80 | 90 |
| I-119 | 80 | 90 |
| I-120 | 80 | 80 |
| I-122 | 80 | 90 |
| I-126 | 80 | 80 |
| I-130 | 80 | 80 |
| I-139 | 80 | 80 |
| I-141 | 80 | 80 |
| I-144 | 80 | 90 |
| I-145 | 80 | 80 |
| I-146 | 80 | 90 |
| I-150 | 80 | 80 |
| I-151 | 80 | 90 |
| I-152 | 80 | 80 |
| I-153 | 80 | 90 |
| I-156 | 80 | 80 |
| I-167 | 80 | 80 |
| I-171 | 80 | 80 |
| I-174 | 80 | 80 |
| I-175 | 80 | 80 |
| I-177 | 80 | 90 |
| I-179 | 80 | 90 |
| I-181 | 80 | 80 |
| I-182 | 80 | 80 |
| I-183 | 80 | 90 |
| I-184 | 80 | 80 |
| I-19 | 80 | 80 |
| I-191 | 80 | 80 |
| I-192 | 80 | 80 |
| I-193 | 80 | 80 |
| I-194 | 80 | 80 |
| I-196 | 80 | 80 |
| I-197 | 80 | 90 |
| I-198 | 80 | 80 |
| I-199 | 80 | 80 |
| I-20 | 80 | 90 |
| I-204 | 80 | 80 |
| I-217 | 80 | 90 |
| I-218 | 80 | 80 |
| I-221 | 80 | 80 |
| I-227 | 80 | 90 |
| I-229 | 80 | 80 |
| I-235 | 80 | 80 |
| I-247 | 80 | 80 |
| I-26 | 80 | 80 |
| I-267 | 80 | 80 |
| I-27 | 80 | 90 |
| I-28 | 80 | 90 |
| I-287 | 80 | 80 |
| I-291 | 80 | 80 |
| I-302 | 80 | 80 |
| I-304 | 80 | 80 |
| I-305 | 80 | 80 |
| I-306 | 80 | 80 |
| I-307 | 80 | 80 |
| I-309 | 80 | 90 |
| I-310 | 80 | 80 |
| I-313 | 80 | 80 |
| I-314 | 80 | 90 |
| I-315 | 80 | 80 |
| I-318 | 80 | 90 |
| I-320 | 80 | 80 |
| I-321 | 80 | 80 |
| I-322 | 80 | 80 |
| I-324 | 80 | 90 |
| I-326 | 80 | 90 |
| I-327 | 80 | 90 |
| I-328 | 80 | 90 |
| I-330 | 80 | 80 |
| I-331 | 80 | 80 |
| I-332 | 80 | 80 |
| I-334 | 80 | 80 |
| I-335 | 80 | 80 |
| I-338 | 80 | 80 |
| I-339 | 80 | 90 |
| I-340 | 80 | 80 |
| I-341 | 80 | 80 |
| I-342 | 80 | 90 |
| I-343 | 80 | 80 |
| I-344 | 80 | 90 |
| I-35 | 80 | 100 |
| I-350 | 80 | 90 |
| I-351 | 80 | 80 |
| I-354 | 80 | 80 |
| I-36 | 80 | 80 |
| I-37 | 80 | 90 |
| I-370 | 80 | 80 |
| I-373 | 80 | 80 |
| I-374 | 80 | 80 |
| I-375 | 80 | 80 |
| I-377 | 80 | 80 |
| I-40 | 80 | 80 |
| I-41 | 80 | 80 |
| I-42 | 80 | 90 |
| I-43 | 80 | 80 |
| I-44 | 80 | 80 |
| I-45 | 80 | 80 |
| I-46 | 80 | 90 |
| I-48 | 80 | 80 |
| I-51 | 80 | 90 |
| I-53 | 80 | 90 |
| I-54 | 80 | 80 |
| I-56 | 80 | 80 |
| I-59 | 80 | 90 |
| I-60 | 80 | 80 |
| I-64 | 80 | 90 |
| I-66 | 80 | 80 |
| I-70 | 80 | 90 |
| I-71 | 80 | 90 |
| I-72 | 80 | 80 |
| I-73 | 80 | 90 |
| I-74 | 80 | 80 |
| I-75 | 80 | 80 |
| I-76 | 80 | 80 |
| I-77 | 80 | 80 |
| I-78 | 80 | 80 |
| I-79 | 80 | 90 |
| I-80 | 80 | 80 |
| I-81 | 80 | 90 |
| I-83 | 80 | 90 |
| I-84 | 80 | 90 |
| I-85 | 80 | 90 |
| I-87 | 80 | 80 |
| I-88 | 80 | 80 |
| I-90 | 80 | 80 |
| I-91 | 80 | 90 |
| I-93 | 80 | 80 |
| I-94 | 80 | 90 |
| I-95 | 80 | 80 |
| I-96 | 80 | 80 |
| I-97 | 80 | 90 |
| I-98 | 80 | 80 |
| I-99 | 80 | 80 |
| III-17 | 80 | 80 |
| III-18 | 80 | 80 |
| III-19 | 80 | 80 |
| III-22 | 80 | 90 |
| III-23 | 80 | 90 |
| III-27 | 80 | 80 |
| III-28 | 80 | 80 |
| III-30 | 80 | 80 |

TABLE B3b
Post-emergence action at 320 g/ha against AMARE in %

| Example number | Dosage [g/ha] | AMARE |
|---|---|---|
| I-01 | 320 | 80 |
| I-02 | 320 | 80 |
| I-03 | 320 | 90 |
| I-04 | 320 | 90 |
| I-05 | 320 | 90 |
| I-07 | 320 | 90 |
| I-08 | 320 | 90 |
| I-111 | 320 | 90 |
| I-130 | 320 | 90 |
| I-137 | 320 | 80 |
| I-139 | 320 | 90 |
| I-140 | 320 | 90 |
| I-141 | 320 | 90 |
| I-144 | 320 | 90 |
| I-145 | 320 | 80 |
| I-146 | 320 | 90 |
| I-147 | 320 | 90 |
| I-150 | 320 | 80 |
| I-151 | 320 | 100 |
| I-152 | 320 | 80 |
| I-153 | 320 | 90 |
| I-156 | 320 | 90 |
| I-16 | 320 | 80 |
| I-162 | 320 | 80 |
| I-165 | 320 | 80 |
| I-167 | 320 | 80 |
| I-171 | 320 | 80 |
| I-172 | 320 | 80 |
| I-174 | 320 | 80 |
| I-175 | 320 | 80 |
| I-177 | 320 | 90 |
| I-179 | 320 | 90 |
| I-180 | 320 | 90 |
| I-181 | 320 | 90 |
| I-182 | 320 | 90 |
| I-183 | 320 | 90 |
| I-184 | 320 | 80 |
| I-185 | 320 | 80 |
| I-188 | 320 | 80 |
| I-190 | 320 | 80 |
| I-191 | 320 | 80 |
| I-192 | 320 | 80 |
| I-193 | 320 | 80 |
| I-194 | 320 | 90 |
| I-196 | 320 | 80 |
| I-197 | 320 | 90 |
| I-198 | 320 | 90 |
| I-199 | 320 | 90 |
| I-20 | 320 | 90 |
| I-202 | 320 | 80 |
| I-204 | 320 | 80 |
| I-207 | 320 | 80 |
| I-208 | 320 | 80 |
| I-210 | 320 | 80 |
| I-212 | 320 | 80 |
| I-217 | 320 | 90 |
| I-218 | 320 | 80 |
| I-221 | 320 | 90 |
| I-227 | 320 | 90 |
| I-229 | 320 | 80 |
| I-230 | 320 | 90 |
| I-235 | 320 | 80 |
| I-236 | 320 | 80 |
| I-237 | 320 | 90 |
| I-238 | 320 | 80 |
| I-239 | 320 | 80 |
| I-241 | 320 | 80 |
| I-242 | 320 | 80 |
| I-243 | 320 | 80 |
| I-245 | 320 | 80 |
| I-246 | 320 | 80 |
| I-247 | 320 | 80 |
| I-251 | 320 | 90 |
| I-26 | 320 | 80 |
| I-266 | 320 | 90 |
| I-267 | 320 | 80 |
| I-27 | 320 | 90 |
| I-273 | 320 | 80 |
| I-274 | 320 | 90 |
| I-275 | 320 | 80 |
| I-276 | 320 | 90 |
| I-28 | 320 | 90 |
| I-287 | 320 | 80 |
| I-291 | 320 | 80 |
| I-293 | 320 | 80 |
| I-294 | 320 | 80 |
| I-298 | 320 | 80 |
| I-299 | 320 | 80 |
| I-300 | 320 | 80 |
| I-302 | 320 | 90 |
| I-304 | 320 | 90 |
| I-305 | 320 | 90 |
| I-306 | 320 | 80 |
| I-307 | 320 | 90 |
| I-309 | 320 | 90 |
| I-31 | 320 | 80 |
| I-310 | 320 | 80 |
| I-313 | 320 | 90 |
| I-314 | 320 | 90 |
| I-315 | 320 | 80 |
| I-318 | 320 | 90 |
| I-319 | 320 | 80 |
| I-320 | 320 | 80 |
| I-321 | 320 | 80 |
| I-322 | 320 | 80 |
| I-324 | 320 | 90 |
| I-325 | 320 | 80 |
| I-326 | 320 | 90 |
| I-327 | 320 | 90 |
| I-328 | 320 | 90 |
| I-330 | 320 | 80 |
| I-331 | 320 | 80 |
| I-332 | 320 | 80 |
| I-333 | 320 | 80 |
| I-334 | 320 | 80 |
| I-335 | 320 | 80 |
| I-336 | 320 | 80 |
| I-337 | 320 | 80 |
| I-338 | 320 | 80 |
| I-339 | 320 | 90 |
| I-340 | 320 | 90 |
| I-341 | 320 | 80 |
| I-342 | 320 | 90 |
| I-343 | 320 | 80 |
| I-344 | 320 | 90 |
| I-345 | 320 | 80 |
| I-348 | 320 | 80 |
| I-350 | 320 | 90 |
| I-351 | 320 | 80 |
| I-352 | 320 | 80 |
| I-354 | 320 | 80 |
| I-358 | 320 | 90 |
| I-359 | 320 | 90 |
| I-366 | 320 | 80 |
| I-369 | 320 | 80 |
| I-370 | 320 | 80 |
| I-371 | 320 | 80 |
| I-373 | 320 | 80 |
| I-375 | 320 | 80 |
| I-376 | 320 | 80 |
| I-377 | 320 | 90 |
| I-383 | 320 | 80 |
| I-41 | 320 | 90 |
| I-42 | 320 | 90 |
| I-48 | 320 | 80 |
| I-60 | 320 | 80 |
| I-65 | 320 | 80 |
| I-66 | 320 | 90 |
| I-69 | 320 | 80 |
| I-80 | 320 | 80 |
| I-85 | 320 | 90 |
| I-86 | 320 | 90 |
| I-87 | 320 | 80 |
| I-88 | 320 | 80 |
| I-94 | 320 | 90 |
| I-95 | 320 | 90 |
| I-96 | 320 | 80 |
| II-38 | 320 | 80 |
| II-39 | 320 | 80 |
| II-40 | 320 | 80 |
| II-41 | 320 | 80 |
| II-42 | 320 | 90 |
| II-43 | 320 | 90 |
| II-44 | 320 | 90 |
| II-45 | 320 | 80 |
| III-01 | 320 | 80 |
| III-02 | 320 | 80 |
| III-08 | 320 | 80 |
| III-09 | 320 | 80 |
| III-22 | 320 | 90 |
| III-23 | 320 | 90 |
| III-24 | 320 | 100 |
| III-25 | 320 | 80 |
| III-27 | 320 | 80 |
| III-28 | 320 | 80 |
| III-30 | 320 | 90 |

TABLE B4a
Post-emergence action at 80 g/ha against AVEFA in %

| Example number | Dosage [g/ha] | AVEFA |
|---|---|---|
| I-01 | 80 | 90 |
| I-05 | 80 | 90 |
| I-08 | 80 | 80 |
| I-10 | 80 | 80 |
| I-100 | 80 | 100 |
| I-101 | 80 | 80 |
| I-102 | 80 | 90 |
| I-107 | 80 | 90 |
| I-11 | 80 | 80 |
| I-125 | 80 | 90 |
| I-13 | 80 | 80 |
| I-144 | 80 | 90 |
| I-146 | 80 | 90 |
| I-151 | 80 | 90 |
| I-153 | 80 | 80 |
| I-164 | 80 | 80 |
| I-166 | 80 | 90 |
| I-167 | 80 | 80 |
| I-171 | 80 | 90 |
| I-172 | 80 | 80 |
| I-174 | 80 | 80 |
| I-175 | 80 | 80 |
| I-177 | 80 | 90 |
| I-180 | 80 | 80 |
| I-184 | 80 | 80 |
| I-188 | 80 | 80 |
| I-189 | 80 | 90 |
| I-19 | 80 | 90 |
| I-191 | 80 | 80 |
| I-193 | 80 | 80 |
| I-196 | 80 | 80 |
| I-197 | 80 | 90 |
| I-198 | 80 | 80 |
| I-20 | 80 | 90 |
| I-212 | 80 | 80 |
| I-218 | 80 | 80 |
| I-227 | 80 | 90 |
| I-23 | 80 | 90 |
| I-24 | 80 | 90 |
| I-25 | 80 | 90 |
| I-252 | 80 | 80 |
| I-26 | 80 | 100 |
| I-260 | 80 | 80 |
| I-264 | 80 | 80 |
| I-267 | 80 | 90 |
| I-27 | 80 | 100 |
| I-275 | 80 | 90 |
| I-28 | 80 | 100 |
| I-288 | 80 | 80 |
| I-289 | 80 | 80 |
| I-29 | 80 | 80 |
| I-291 | 80 | 80 |
| I-293 | 80 | 80 |
| I-296 | 80 | 80 |
| I-298 | 80 | 80 |
| I-299 | 80 | 80 |
| I-302 | 80 | 80 |
| I-307 | 80 | 90 |
| I-308 | 80 | 90 |
| I-314 | 80 | 90 |
| I-316 | 80 | 90 |
| I-317 | 80 | 90 |
| I-318 | 80 | 90 |
| I-319 | 80 | 80 |
| I-32 | 80 | 90 |
| I-321 | 80 | 90 |
| I-322 | 80 | 80 |
| I-323 | 80 | 90 |
| I-324 | 80 | 100 |
| I-326 | 80 | 90 |
| I-328 | 80 | 90 |
| I-33 | 80 | 90 |
| I-331 | 80 | 90 |
| I-332 | 80 | 80 |
| I-333 | 80 | 80 |
| I-336 | 80 | 80 |
| I-339 | 80 | 90 |
| I-34 | 80 | 90 |
| I-340 | 80 | 90 |
| I-341 | 80 | 80 |
| I-342 | 80 | 90 |
| I-343 | 80 | 80 |
| I-344 | 80 | 90 |
| I-348 | 80 | 80 |
| I-35 | 80 | 100 |
| I-350 | 80 | 90 |
| I-358 | 80 | 90 |
| I-366 | 80 | 90 |
| I-374 | 80 | 90 |
| I-375 | 80 | 90 |
| I-380 | 80 | 90 |
| I-382 | 80 | 90 |
| I-383 | 80 | 80 |
| I-40 | 80 | 80 |
| I-41 | 80 | 90 |
| I-42 | 80 | 100 |
| I-43 | 80 | 90 |
| I-44 | 80 | 90 |
| I-45 | 80 | 80 |
| I-46 | 80 | 80 |
| I-51 | 80 | 90 |
| I-52 | 80 | 90 |
| I-56 | 80 | 80 |
| I-59 | 80 | 80 |
| I-64 | 80 | 80 |
| I-69 | 80 | 80 |
| I-73 | 80 | 80 |
| I-74 | 80 | 90 |
| I-75 | 80 | 80 |
| I-76 | 80 | 90 |
| I-77 | 80 | 90 |
| I-79 | 80 | 100 |
| I-80 | 80 | 80 |
| I-81 | 80 | 90 |
| I-83 | 80 | 100 |
| I-84 | 80 | 80 |
| I-87 | 80 | 80 |
| I-91 | 80 | 90 |
| I-94 | 80 | 80 |
| I-95 | 80 | 80 |
| I-96 | 80 | 80 |
| I-97 | 80 | 80 |
| I-98 | 80 | 80 |
| III-22 | 80 | 100 |

TABLE B4b
Post-emergence action at 320 g/ha against AVEFA in %

| Example number | Dosage [g/ha] | AVEFA |
|---|---|---|
| I-01 | 320 | 100 |
| I-02 | 320 | 80 |
| I-03 | 320 | 90 |
| I-05 | 320 | 90 |
| I-07 | 320 | 90 |
| I-08 | 320 | 90 |
| I-10 | 320 | 90 |
| I-11 | 320 | 90 |
| I-13 | 320 | 90 |
| I-130 | 320 | 90 |
| I-133 | 320 | 80 |
| I-140 | 320 | 90 |
| I-141 | 320 | 80 |
| I-144 | 320 | 90 |
| I-145 | 320 | 90 |
| I-146 | 320 | 90 |
| I-148 | 320 | 80 |
| I-149 | 320 | 80 |
| I-151 | 320 | 90 |
| I-153 | 320 | 90 |
| I-154 | 320 | 80 |
| I-155 | 320 | 80 |
| I-156 | 320 | 90 |
| I-164 | 320 | 80 |
| I-165 | 320 | 90 |
| I-166 | 320 | 90 |
| I-167 | 320 | 90 |
| I-168 | 320 | 80 |
| I-169 | 320 | 80 |
| I-17 | 320 | 80 |
| I-171 | 320 | 90 |
| I-172 | 320 | 90 |
| I-174 | 320 | 90 |
| I-175 | 320 | 80 |
| I-177 | 320 | 90 |
| I-179 | 320 | 90 |
| I-18 | 320 | 80 |
| I-180 | 320 | 90 |
| I-181 | 320 | 90 |
| I-182 | 320 | 90 |
| I-183 | 320 | 90 |
| I-184 | 320 | 90 |
| I-185 | 320 | 80 |
| I-186 | 320 | 80 |
| I-187 | 320 | 80 |
| I-188 | 320 | 80 |
| I-189 | 320 | 90 |
| I-190 | 320 | 80 |
| I-191 | 320 | 90 |
| I-192 | 320 | 80 |
| I-193 | 320 | 90 |
| I-194 | 320 | 90 |
| I-195 | 320 | 90 |
| I-196 | 320 | 90 |
| I-197 | 320 | 90 |
| I-198 | 320 | 80 |
| I-199 | 320 | 80 |
| I-20 | 320 | 90 |
| I-204 | 320 | 80 |
| I-212 | 320 | 90 |
| I-216 | 320 | 80 |
| I-217 | 320 | 90 |
| I-218 | 320 | 90 |
| I-221 | 320 | 80 |
| I-223 | 320 | 80 |
| I-225 | 320 | 80 |
| I-227 | 320 | 90 |
| I-237 | 320 | 80 |
| I-242 | 320 | 80 |
| I-245 | 320 | 80 |
| I-251 | 320 | 90 |
| I-252 | 320 | 80 |
| I-26 | 320 | 100 |
| I-260 | 320 | 80 |
| I-264 | 320 | 90 |
| I-267 | 320 | 90 |
| I-27 | 320 | 100 |
| I-272 | 320 | 80 |
| I-274 | 320 | 90 |
| I-275 | 320 | 90 |
| I-28 | 320 | 100 |
| I-285 | 320 | 80 |
| I-287 | 320 | 90 |
| I-288 | 320 | 90 |
| I-289 | 320 | 90 |
| I-291 | 320 | 80 |
| I-293 | 320 | 90 |
| I-294 | 320 | 90 |
| I-296 | 320 | 80 |
| I-298 | 320 | 90 |
| I-299 | 320 | 80 |
| I-302 | 320 | 80 |
| I-304 | 320 | 90 |
| I-305 | 320 | 80 |
| I-306 | 320 | 80 |
| I-307 | 320 | 90 |
| I-308 | 320 | 90 |
| I-310 | 320 | 100 |
| I-311 | 320 | 80 |
| I-313 | 320 | 80 |
| I-314 | 320 | 90 |
| I-315 | 320 | 80 |
| I-316 | 320 | 100 |
| I-317 | 320 | 90 |
| I-318 | 320 | 90 |
| I-319 | 320 | 80 |
| I-320 | 320 | 80 |
| I-321 | 320 | 90 |
| I-322 | 320 | 80 |
| I-323 | 320 | 90 |
| I-324 | 320 | 100 |
| I-326 | 320 | 90 |
| I-328 | 320 | 90 |
| I-330 | 320 | 80 |
| I-331 | 320 | 90 |
| I-332 | 320 | 90 |
| I-333 | 320 | 90 |
| I-334 | 320 | 80 |
| I-335 | 320 | 90 |
| I-336 | 320 | 90 |
| I-337 | 320 | 80 |
| I-338 | 320 | 90 |
| I-339 | 320 | 90 |
| I-340 | 320 | 90 |
| I-341 | 320 | 80 |
| I-342 | 320 | 100 |
| I-343 | 320 | 80 |
| I-344 | 320 | 90 |
| I-348 | 320 | 80 |
| I-350 | 320 | 90 |
| I-352 | 320 | 80 |
| I-354 | 320 | 80 |
| I-358 | 320 | 90 |
| I-359 | 320 | 90 |
| I-360 | 320 | 80 |
| I-366 | 320 | 90 |
| I-369 | 320 | 80 |
| I-370 | 320 | 90 |
| I-375 | 320 | 90 |
| I-380 | 320 | 90 |
| I-382 | 320 | 90 |
| I-383 | 320 | 80 |
| I-388 | 320 | 80 |
| I-41 | 320 | 90 |
| I-42 | 320 | 100 |
| I-48 | 320 | 100 |
| I-58 | 320 | 90 |
| I-60 | 320 | 90 |
| I-65 | 320 | 80 |
| I-66 | 320 | 100 |
| I-69 | 320 | 100 |
| I-80 | 320 | 80 |
| I-85 | 320 | 100 |
| I-86 | 320 | 100 |
| I-87 | 320 | 90 |
| I-88 | 320 | 90 |
| I-94 | 320 | 100 |
| I-95 | 320 | 80 |
| I-96 | 320 | 90 |
| II-38 | 320 | 90 |
| II-39 | 320 | 90 |
| II-40 | 320 | 90 |
| II-41 | 320 | 90 |
| II-42 | 320 | 90 |
| II-43 | 320 | 90 |
| II-45 | 320 | 90 |
| III-01 | 320 | 80 |
| III-22 | 320 | 100 |
| III-23 | 320 | 80 |
| III-24 | 320 | 80 |

TABLE B5a
Post-emergence action at 80 g/ha against DIGSA in %

| Example number | Dosage [g/ha] | DIGSA |
|---|---|---|
| I-07 | 80 | 90 |
| I-10 | 80 | 90 |
| I-11 | 80 | 90 |
| I-118 | 80 | 80 |
| I-121 | 80 | 80 |
| I-13 | 80 | 90 |
| I-130 | 80 | 80 |
| I-141 | 80 | 90 |
| I-144 | 80 | 90 |
| I-148 | 80 | 80 |
| I-149 | 80 | 80 |
| I-15 | 80 | 90 |
| I-154 | 80 | 80 |
| I-164 | 80 | 80 |
| I-165 | 80 | 90 |
| I-166 | 80 | 90 |
| I-167 | 80 | 90 |
| I-168 | 80 | 80 |
| I-17 | 80 | 90 |
| I-172 | 80 | 80 |
| I-174 | 80 | 90 |
| I-175 | 80 | 90 |
| I-176 | 80 | 80 |
| I-179 | 80 | 90 |
| I-18 | 80 | 90 |
| I-180 | 80 | 80 |
| I-181 | 80 | 90 |
| I-182 | 80 | 90 |
| I-19 | 80 | 80 |
| I-192 | 80 | 80 |
| I-194 | 80 | 80 |
| I-195 | 80 | 90 |
| I-197 | 80 | 90 |
| I-208 | 80 | 80 |
| I-212 | 80 | 90 |
| I-216 | 80 | 80 |
| I-223 | 80 | 80 |
| I-227 | 80 | 80 |
| I-247 | 80 | 80 |
| I-251 | 80 | 80 |
| I-252 | 80 | 90 |
| I-260 | 80 | 90 |
| I-264 | 80 | 80 |
| I-285 | 80 | 80 |
| I-287 | 80 | 80 |
| I-288 | 80 | 80 |
| I-29 | 80 | 90 |
| I-290 | 80 | 90 |
| I-294 | 80 | 80 |
| I-296 | 80 | 80 |
| I-299 | 80 | 80 |
| I-30 | 80 | 90 |
| I-304 | 80 | 90 |
| I-314 | 80 | 90 |
| I-318 | 80 | 90 |
| I-322 | 80 | 80 |
| I-326 | 80 | 80 |
| I-328 | 80 | 90 |
| I-344 | 80 | 90 |
| I-348 | 80 | 80 |
| I-36 | 80 | 90 |
| I-360 | 80 | 80 |
| I-366 | 80 | 80 |
| I-37 | 80 | 90 |
| I-38 | 80 | 90 |
| I-380 | 80 | 80 |
| I-382 | 80 | 80 |
| I-383 | 80 | 80 |
| I-39 | 80 | 90 |
| I-45 | 80 | 90 |
| I-46 | 80 | 90 |
| I-56 | 80 | 90 |
| I-64 | 80 | 90 |
| I-90 | 80 | 90 |
| I-97 | 80 | 80 |

TABLE B5b
Post-emergence action at 320 g/ha against DIGSA in %

| Example number | Dosage [g/ha] | DIGSA |
|---|---|---|
| I-04 | 320 | 80 |
| I-05 | 320 | 90 |
| I-07 | 320 | 90 |
| I-08 | 320 | 90 |
| I-10 | 320 | 90 |
| I-11 | 320 | 90 |
| I-13 | 320 | 90 |
| I-130 | 320 | 90 |
| I-133 | 320 | 80 |
| I-134 | 320 | 80 |
| I-136 | 320 | 80 |
| I-139 | 320 | 90 |
| I-140 | 320 | 80 |
| I-141 | 320 | 90 |
| I-144 | 320 | 90 |
| I-145 | 320 | 90 |
| I-146 | 320 | 90 |
| I-148 | 320 | 90 |
| I-149 | 320 | 90 |
| I-15 | 320 | 90 |
| I-154 | 320 | 90 |
| I-159 | 320 | 80 |
| I-163 | 320 | 80 |
| I-164 | 320 | 90 |
| I-165 | 320 | 90 |
| I-166 | 320 | 90 |
| I-167 | 320 | 90 |
| I-168 | 320 | 90 |
| I-169 | 320 | 90 |
| I-17 | 320 | 90 |
| I-171 | 320 | 80 |
| I-172 | 320 | 90 |
| I-174 | 320 | 90 |
| I-175 | 320 | 90 |
| I-176 | 320 | 90 |
| I-177 | 320 | 90 |
| I-179 | 320 | 90 |
| I-18 | 320 | 90 |
| I-180 | 320 | 80 |
| I-181 | 320 | 90 |
| I-182 | 320 | 100 |
| I-192 | 320 | 80 |
| I-194 | 320 | 90 |
| I-195 | 320 | 90 |
| I-197 | 320 | 90 |
| I-208 | 320 | 90 |
| I-212 | 320 | 90 |
| I-216 | 320 | 80 |
| I-221 | 320 | 80 |
| I-223 | 320 | 90 |
| I-227 | 320 | 90 |
| I-231 | 320 | 90 |
| I-237 | 320 | 90 |
| I-242 | 320 | 80 |
| I-244 | 320 | 80 |
| I-247 | 320 | 80 |
| I-251 | 320 | 90 |
| I-252 | 320 | 90 |
| I-260 | 320 | 90 |
| I-264 | 320 | 80 |
| I-266 | 320 | 90 |
| I-267 | 320 | 90 |
| I-274 | 320 | 90 |
| I-275 | 320 | 90 |
| I-285 | 320 | 90 |
| I-286 | 320 | 90 |
| I-287 | 320 | 80 |
| I-288 | 320 | 90 |
| I-289 | 320 | 80 |
| I-290 | 320 | 90 |
| I-293 | 320 | 80 |
| I-294 | 320 | 90 |
| I-296 | 320 | 90 |
| I-298 | 320 | 80 |
| I-299 | 320 | 90 |
| I-300 | 320 | 80 |
| I-302 | 320 | 80 |
| I-304 | 320 | 90 |
| I-314 | 320 | 90 |
| I-318 | 320 | 90 |
| I-322 | 320 | 80 |
| I-326 | 320 | 90 |
| I-328 | 320 | 90 |
| I-344 | 320 | 90 |
| I-347 | 320 | 80 |
| I-348 | 320 | 90 |
| I-360 | 320 | 80 |
| I-366 | 320 | 90 |
| I-380 | 320 | 90 |
| I-382 | 320 | 90 |
| I-383 | 320 | 90 |
| I-66 | 320 | 90 |
| II-41 | 320 | 80 |
| II-42 | 320 | 80 |
| II-43 | 320 | 80 |
| II-44 | 320 | 80 |
| II-45 | 320 | 80 |
| III-29 | 320 | 90 |

TABLE B6a
Post-emergence action at 80 g/ha against ECHCG in %

| Example number | Dosage [g/ha] | ECHCG |
|---|---|---|
| I-01 | 80 | 80 |
| I-02 | 80 | 90 |
| I-08 | 80 | 80 |
| I-100 | 80 | 90 |
| I-116 | 80 | 80 |
| I-118 | 80 | 80 |
| I-119 | 80 | 80 |
| I-121 | 80 | 80 |
| I-125 | 80 | 90 |
| I-126 | 80 | 80 |
| I-141 | 80 | 80 |
| I-144 | 80 | 90 |
| I-145 | 80 | 90 |
| I-146 | 80 | 90 |
| I-148 | 80 | 80 |
| I-149 | 80 | 80 |
| I-151 | 80 | 90 |
| I-152 | 80 | 80 |
| I-153 | 80 | 80 |
| I-154 | 80 | 80 |
| I-155 | 80 | 80 |
| I-156 | 80 | 80 |
| I-157 | 80 | 80 |
| I-159 | 80 | 80 |
| I-16 | 80 | 80 |
| I-163 | 80 | 80 |
| I-164 | 80 | 80 |
| I-165 | 80 | 90 |
| I-166 | 80 | 80 |
| I-167 | 80 | 80 |
| I-168 | 80 | 80 |
| I-169 | 80 | 80 |
| I-17 | 80 | 80 |
| I-171 | 80 | 80 |
| I-172 | 80 | 80 |
| I-174 | 80 | 80 |
| I-175 | 80 | 80 |
| I-176 | 80 | 80 |
| I-177 | 80 | 100 |
| I-179 | 80 | 90 |
| I-18 | 80 | 80 |
| I-180 | 80 | 80 |
| I-181 | 80 | 90 |
| I-182 | 80 | 90 |
| I-183 | 80 | 90 |
| I-184 | 80 | 90 |
| I-185 | 80 | 80 |
| I-186 | 80 | 80 |
| I-187 | 80 | 80 |
| I-188 | 80 | 80 |
| I-189 | 80 | 90 |
| I-190 | 80 | 80 |
| I-191 | 80 | 80 |
| I-192 | 80 | 90 |
| I-193 | 80 | 90 |
| I-194 | 80 | 80 |
| I-196 | 80 | 80 |
| I-197 | 80 | 90 |
| I-198 | 80 | 80 |
| I-199 | 80 | 90 |
| I-202 | 80 | 80 |
| I-204 | 80 | 80 |
| I-207 | 80 | 80 |
| I-208 | 80 | 80 |
| I-210 | 80 | 80 |
| I-211 | 80 | 80 |
| I-212 | 80 | 90 |
| I-216 | 80 | 80 |
| I-217 | 80 | 90 |
| I-218 | 80 | 80 |
| I-221 | 80 | 80 |
| I-223 | 80 | 80 |
| I-225 | 80 | 80 |
| I-227 | 80 | 90 |
| I-229 | 80 | 80 |
| I-23 | 80 | 80 |
| I-231 | 80 | 80 |
| I-24 | 80 | 80 |
| I-247 | 80 | 80 |
| I-251 | 80 | 80 |
| I-252 | 80 | 80 |
| I-26 | 80 | 80 |
| I-264 | 80 | 90 |
| I-266 | 80 | 80 |
| I-27 | 80 | 90 |
| I-274 | 80 | 80 |
| I-28 | 80 | 80 |
| I-287 | 80 | 90 |
| I-289 | 80 | 80 |
| I-29 | 80 | 80 |
| I-290 | 80 | 80 |
| I-291 | 80 | 80 |
| I-294 | 80 | 80 |
| I-296 | 80 | 80 |
| I-298 | 80 | 80 |
| I-299 | 80 | 80 |
| I-30 | 80 | 80 |
| I-300 | 80 | 80 |
| I-304 | 80 | 80 |
| I-305 | 80 | 90 |
| I-307 | 80 | 90 |
| I-308 | 80 | 90 |
| I-311 | 80 | 80 |
| I-314 | 80 | 90 |
| I-315 | 80 | 90 |
| I-316 | 80 | 90 |
| I-317 | 80 | 80 |
| I-318 | 80 | 90 |
| I-319 | 80 | 80 |
| I-32 | 80 | 80 |
| I-321 | 80 | 90 |
| I-323 | 80 | 90 |
| I-324 | 80 | 90 |
| I-325 | 80 | 90 |
| I-326 | 80 | 90 |
| I-327 | 80 | 80 |
| I-328 | 80 | 90 |
| I-33 | 80 | 90 |
| I-330 | 80 | 80 |
| I-331 | 80 | 90 |
| I-332 | 80 | 80 |
| I-333 | 80 | 80 |
| I-334 | 80 | 80 |
| I-335 | 80 | 80 |
| I-336 | 80 | 80 |
| I-337 | 80 | 80 |
| I-339 | 80 | 90 |
| I-340 | 80 | 80 |
| I-341 | 80 | 100 |
| I-342 | 80 | 90 |
| I-343 | 80 | 90 |
| I-344 | 80 | 90 |
| I-348 | 80 | 90 |
| I-35 | 80 | 90 |
| I-350 | 80 | 80 |
| I-36 | 80 | 90 |
| I-360 | 80 | 80 |
| I-366 | 80 | 90 |
| I-369 | 80 | 80 |
| I-37 | 80 | 80 |
| I-370 | 80 | 80 |
| I-375 | 80 | 90 |
| I-377 | 80 | 80 |
| I-38 | 80 | 90 |
| I-380 | 80 | 80 |
| I-382 | 80 | 80 |
| I-383 | 80 | 80 |
| I-388 | 80 | 90 |
| I-39 | 80 | 80 |
| I-40 | 80 | 90 |
| I-42 | 80 | 90 |
| I-43 | 80 | 90 |
| I-47 | 80 | 80 |
| I-48 | 80 | 80 |
| I-51 | 80 | 90 |
| I-53 | 80 | 80 |
| I-59 | 80 | 80 |
| I-64 | 80 | 90 |
| I-65 | 80 | 80 |
| I-66 | 80 | 80 |
| I-73 | 80 | 80 |
| I-74 | 80 | 90 |
| I-75 | 80 | 90 |
| I-76 | 80 | 90 |
| I-77 | 80 | 90 |
| I-80 | 80 | 80 |
| I-81 | 80 | 90 |
| I-83 | 80 | 90 |
| I-84 | 80 | 80 |
| I-85 | 80 | 90 |
| I-86 | 80 | 100 |
| I-88 | 80 | 80 |
| I-91 | 80 | 80 |
| I-96 | 80 | 90 |
| I-97 | 80 | 80 |
| I-98 | 80 | 80 |
| III-22 | 80 | 100 |
| III-24 | 80 | 80 |
| III-29 | 80 | 80 |

TABLE B6b
Post-emergence action at 320 g/ha against ECHCG in %

| Example number | Dosage [g/ha] | ECHCG |
|---|---|---|
| I-01 | 320 | 90 |
| I-02 | 320 | 90 |
| I-03 | 320 | 90 |
| I-05 | 320 | 90 |
| I-07 | 320 | 90 |
| I-08 | 320 | 90 |
| I-10 | 320 | 80 |
| I-11 | 320 | 80 |
| I-111 | 320 | 90 |
| I-130 | 320 | 90 |
| I-133 | 320 | 90 |
| I-134 | 320 | 80 |
| I-136 | 320 | 80 |
| I-137 | 320 | 80 |
| I-138 | 320 | 90 |
| I-139 | 320 | 80 |
| I-140 | 320 | 80 |
| I-141 | 320 | 80 |
| I-144 | 320 | 90 |
| I-145 | 320 | 90 |
| I-146 | 320 | 90 |
| I-148 | 320 | 80 |
| I-149 | 320 | 80 |
| I-151 | 320 | 90 |
| I-152 | 320 | 80 |
| I-153 | 320 | 90 |
| I-154 | 320 | 80 |
| I-155 | 320 | 80 |
| I-156 | 320 | 80 |
| I-157 | 320 | 90 |
| I-159 | 320 | 90 |
| I-16 | 320 | 80 |
| I-162 | 320 | 80 |
| I-163 | 320 | 80 |
| I-164 | 320 | 80 |
| I-165 | 320 | 90 |
| I-166 | 320 | 80 |
| I-167 | 320 | 80 |
| I-168 | 320 | 90 |
| I-169 | 320 | 90 |
| I-17 | 320 | 80 |
| I-171 | 320 | 80 |
| I-172 | 320 | 90 |
| I-174 | 320 | 80 |
| I-175 | 320 | 80 |
| I-176 | 320 | 90 |
| I-177 | 320 | 100 |
| I-179 | 320 | 90 |
| I-18 | 320 | 80 |
| I-180 | 320 | 90 |
| I-181 | 320 | 100 |
| I-182 | 320 | 90 |
| I-183 | 320 | 90 |
| I-184 | 320 | 90 |
| I-185 | 320 | 90 |
| I-186 | 320 | 90 |
| I-187 | 320 | 80 |
| I-188 | 320 | 90 |
| I-189 | 320 | 90 |
| I-190 | 320 | 80 |
| I-191 | 320 | 80 |
| I-192 | 320 | 90 |
| I-193 | 320 | 90 |
| I-194 | 320 | 80 |
| I-195 | 320 | 80 |
| I-196 | 320 | 80 |
| I-197 | 320 | 90 |
| I-198 | 320 | 80 |
| I-199 | 320 | 90 |
| I-20 | 320 | 80 |
| I-202 | 320 | 90 |
| I-204 | 320 | 80 |
| I-206 | 320 | 80 |
| I-207 | 320 | 80 |
| I-208 | 320 | 90 |
| I-210 | 320 | 80 |
| I-211 | 320 | 80 |
| I-212 | 320 | 90 |
| I-216 | 320 | 80 |
| I-217 | 320 | 90 |
| I-218 | 320 | 90 |
| I-221 | 320 | 80 |
| I-223 | 320 | 90 |
| I-225 | 320 | 90 |
| I-227 | 320 | 100 |
| I-229 | 320 | 80 |
| I-230 | 320 | 80 |
| I-231 | 320 | 90 |
| I-238 | 320 | 80 |
| I-247 | 320 | 80 |
| I-251 | 320 | 90 |
| I-252 | 320 | 90 |
| I-26 | 320 | 80 |
| I-260 | 320 | 90 |
| I-264 | 320 | 90 |
| I-266 | 320 | 90 |
| I-267 | 320 | 90 |
| I-27 | 320 | 90 |
| I-272 | 320 | 80 |
| I-273 | 320 | 90 |
| I-274 | 320 | 90 |
| I-275 | 320 | 90 |
| I-276 | 320 | 80 |
| I-277 | 320 | 80 |
| I-278 | 320 | 80 |
| I-279 | 320 | 80 |
| I-28 | 320 | 90 |
| I-285 | 320 | 80 |
| I-286 | 320 | 80 |
| I-287 | 320 | 90 |
| I-288 | 320 | 80 |
| I-289 | 320 | 80 |
| I-290 | 320 | 90 |
| I-291 | 320 | 80 |
| I-293 | 320 | 80 |
| I-294 | 320 | 90 |
| I-296 | 320 | 80 |
| I-298 | 320 | 90 |
| I-299 | 320 | 90 |
| I-300 | 320 | 90 |
| I-302 | 320 | 80 |
| I-304 | 320 | 90 |
| I-305 | 320 | 90 |
| I-306 | 320 | 90 |
| I-307 | 320 | 90 |
| I-308 | 320 | 90 |
| I-309 | 320 | 90 |
| I-310 | 320 | 90 |
| I-311 | 320 | 90 |
| I-312 | 320 | 90 |
| I-313 | 320 | 90 |
| I-314 | 320 | 90 |
| I-315 | 320 | 100 |
| I-316 | 320 | 90 |
| I-317 | 320 | 90 |
| I-318 | 320 | 90 |
| I-319 | 320 | 90 |
| I-320 | 320 | 90 |
| I-321 | 320 | 90 |
| I-322 | 320 | 80 |
| I-323 | 320 | 90 |
| I-324 | 320 | 90 |
| I-325 | 320 | 90 |
| I-326 | 320 | 90 |
| I-327 | 320 | 90 |
| I-328 | 320 | 90 |
| I-330 | 320 | 90 |
| I-331 | 320 | 90 |
| I-332 | 320 | 90 |
| I-333 | 320 | 90 |
| I-334 | 320 | 90 |
| I-335 | 320 | 90 |
| I-336 | 320 | 80 |
| I-337 | 320 | 90 |
| I-338 | 320 | 80 |
| I-339 | 320 | 90 |
| I-340 | 320 | 90 |
| I-341 | 320 | 100 |
| I-342 | 320 | 90 |
| I-343 | 320 | 90 |
| I-344 | 320 | 90 |
| I-347 | 320 | 80 |
| I-348 | 320 | 90 |
| I-350 | 320 | 90 |
| I-351 | 320 | 80 |
| I-352 | 320 | 80 |
| I-354 | 320 | 100 |
| I-358 | 320 | 90 |
| I-359 | 320 | 80 |
| I-360 | 320 | 80 |
| I-362 | 320 | 80 |
| I-363 | 320 | 80 |
| I-366 | 320 | 90 |
| I-369 | 320 | 80 |
| I-370 | 320 | 80 |
| I-373 | 320 | 90 |
| I-375 | 320 | 90 |
| I-376 | 320 | 90 |
| I-377 | 320 | 90 |
| I-378 | 320 | 80 |
| I-380 | 320 | 90 |
| I-381 | 320 | 80 |
| I-382 | 320 | 90 |
| I-383 | 320 | 90 |
| I-388 | 320 | 90 |
| I-41 | 320 | 80 |
| I-42 | 320 | 90 |
| I-48 | 320 | 90 |
| I-58 | 320 | 80 |
| I-60 | 320 | 80 |
| I-65 | 320 | 80 |
| I-66 | 320 | 90 |
| I-80 | 320 | 80 |
| I-82 | 320 | 80 |
| I-85 | 320 | 90 |
| I-86 | 320 | 100 |
| I-87 | 320 | 80 |
| I-88 | 320 | 90 |
| I-94 | 320 | 90 |
| I-95 | 320 | 90 |
| I-96 | 320 | 90 |
| II-37 | 320 | 80 |
| II-38 | 320 | 90 |
| II-39 | 320 | 90 |
| II-40 | 320 | 90 |
| II-41 | 320 | 90 |
| II-42 | 320 | 90 |
| II-43 | 320 | 90 |
| II-44 | 320 | 90 |
| II-45 | 320 | 90 |
| III-01 | 320 | 80 |
| III-22 | 320 | 100 |
| III-24 | 320 | 100 |
| III-29 | 320 | 80 |

TABLE B7a
Post-emergence action at 80 g/ha against LOLRI in %

| Example number | Dosage [g/ha] | LOLRI |
|---|---|---|
| I-01 | 80 | 80 |
| I-02 | 80 | 80 |
| I-10 | 80 | 90 |
| I-100 | 80 | 80 |
| I-101 | 80 | 90 |
| I-102 | 80 | 90 |
| I-107 | 80 | 80 |
| I-125 | 80 | 80 |
| I-126 | 80 | 80 |
| I-134 | 80 | 80 |
| I-144 | 80 | 90 |
| I-145 | 80 | 90 |
| I-148 | 80 | 80 |
| I-149 | 80 | 80 |
| I-154 | 80 | 80 |
| I-164 | 80 | 80 |
| I-166 | 80 | 80 |
| I-167 | 80 | 80 |
| I-171 | 80 | 80 |
| I-172 | 80 | 90 |
| I-174 | 80 | 80 |
| I-175 | 80 | 80 |
| I-176 | 80 | 80 |
| I-177 | 80 | 100 |
| I-180 | 80 | 90 |
| I-184 | 80 | 90 |
| I-185 | 80 | 80 |
| I-187 | 80 | 90 |
| I-188 | 80 | 90 |
| I-189 | 80 | 100 |
| I-19 | 80 | 80 |
| I-190 | 80 | 90 |
| I-191 | 80 | 90 |
| I-194 | 80 | 80 |
| I-196 | 80 | 90 |
| I-197 | 80 | 90 |
| I-198 | 80 | 80 |
| I-199 | 80 | 90 |
| I-20 | 80 | 90 |
| I-218 | 80 | 80 |
| I-221 | 80 | 80 |
| I-227 | 80 | 80 |
| I-24 | 80 | 90 |
| I-25 | 80 | 80 |
| I-251 | 80 | 90 |
| I-252 | 80 | 80 |
| I-264 | 80 | 90 |
| I-267 | 80 | 90 |
| I-27 | 80 | 90 |
| I-274 | 80 | 80 |
| I-275 | 80 | 90 |
| I-285 | 80 | 80 |
| I-287 | 80 | 90 |
| I-288 | 80 | 80 |
| I-289 | 80 | 80 |
| I-291 | 80 | 80 |
| I-293 | 80 | 80 |
| I-296 | 80 | 80 |
| I-298 | 80 | 90 |
| I-299 | 80 | 80 |
| I-300 | 80 | 90 |
| I-307 | 80 | 100 |
| I-308 | 80 | 90 |
| I-314 | 80 | 90 |
| I-316 | 80 | 100 |
| I-317 | 80 | 90 |
| I-318 | 80 | 90 |
| I-319 | 80 | 80 |
| I-32 | 80 | 90 |
| I-321 | 80 | 100 |
| I-322 | 80 | 80 |
| I-323 | 80 | 90 |
| I-326 | 80 | 90 |
| I-328 | 80 | 90 |
| I-33 | 80 | 80 |
| I-331 | 80 | 90 |
| I-332 | 80 | 80 |
| I-336 | 80 | 80 |
| I-339 | 80 | 90 |
| I-34 | 80 | 80 |
| I-340 | 80 | 90 |
| I-341 | 80 | 90 |
| I-342 | 80 | 90 |
| I-344 | 80 | 90 |
| I-348 | 80 | 80 |
| I-35 | 80 | 90 |
| I-350 | 80 | 80 |
| I-358 | 80 | 90 |
| I-366 | 80 | 90 |
| I-369 | 80 | 80 |
| I-374 | 80 | 90 |
| I-375 | 80 | 90 |
| I-380 | 80 | 90 |
| I-383 | 80 | 80 |
| I-388 | 80 | 80 |
| I-40 | 80 | 90 |
| I-41 | 80 | 80 |
| I-42 | 80 | 100 |
| I-52 | 80 | 80 |
| I-59 | 80 | 80 |
| I-64 | 80 | 80 |
| I-73 | 80 | 80 |
| I-74 | 80 | 90 |
| I-75 | 80 | 90 |
| I-76 | 80 | 80 |
| I-83 | 80 | 90 |
| I-84 | 80 | 90 |
| I-85 | 80 | 100 |
| I-86 | 80 | 100 |
| I-91 | 80 | 90 |
| I-96 | 80 | 90 |
| III-24 | 80 | 100 |

TABLE B7b
Post-emergence action at 320 g/ha against LOLRI in %

| Example number | Dosage [g/ha] | LOLRI |
|---|---|---|
| I-01 | 320 | 90 |
| I-02 | 320 | 80 |
| I-05 | 320 | 90 |
| I-07 | 320 | 90 |
| I-08 | 320 | 90 |
| I-10 | 320 | 90 |
| I-11 | 320 | 80 |
| I-13 | 320 | 90 |
| I-130 | 320 | 90 |
| I-133 | 320 | 90 |
| I-134 | 320 | 90 |
| I-136 | 320 | 80 |
| I-137 | 320 | 90 |
| I-138 | 320 | 90 |
| I-140 | 320 | 90 |
| I-141 | 320 | 90 |
| I-144 | 320 | 90 |
| I-145 | 320 | 90 |
| I-146 | 320 | 90 |
| I-148 | 320 | 80 |
| I-149 | 320 | 90 |
| I-15 | 320 | 80 |
| I-151 | 320 | 90 |
| I-154 | 320 | 90 |
| I-155 | 320 | 80 |
| I-159 | 320 | 80 |
| I-163 | 320 | 80 |
| I-164 | 320 | 90 |
| I-165 | 320 | 100 |
| I-166 | 320 | 90 |
| I-167 | 320 | 80 |
| I-168 | 320 | 80 |
| I-169 | 320 | 80 |
| I-17 | 320 | 80 |
| I-171 | 320 | 90 |
| I-172 | 320 | 90 |
| I-174 | 320 | 80 |
| I-175 | 320 | 80 |
| I-176 | 320 | 80 |
| I-177 | 320 | 100 |
| I-18 | 320 | 80 |
| I-180 | 320 | 90 |
| I-181 | 320 | 80 |
| I-183 | 320 | 90 |
| I-184 | 320 | 90 |
| I-185 | 320 | 80 |
| I-187 | 320 | 90 |
| I-188 | 320 | 90 |
| I-189 | 320 | 100 |
| I-190 | 320 | 90 |
| I-191 | 320 | 90 |
| I-192 | 320 | 80 |
| I-193 | 320 | 90 |
| I-194 | 320 | 90 |
| I-195 | 320 | 80 |
| I-196 | 320 | 90 |
| I-197 | 320 | 90 |
| I-198 | 320 | 80 |
| I-199 | 320 | 90 |
| I-20 | 320 | 90 |
| I-208 | 320 | 80 |
| I-210 | 320 | 80 |
| I-211 | 320 | 80 |
| I-212 | 320 | 80 |
| I-217 | 320 | 80 |
| I-218 | 320 | 90 |
| I-221 | 320 | 90 |
| I-227 | 320 | 90 |
| I-237 | 320 | 80 |
| I-238 | 320 | 80 |
| I-240 | 320 | 80 |
| I-242 | 320 | 80 |
| I-244 | 320 | 80 |
| I-247 | 320 | 80 |
| I-251 | 320 | 90 |
| I-252 | 320 | 80 |
| I-26 | 320 | 80 |
| I-260 | 320 | 80 |
| I-264 | 320 | 90 |
| I-266 | 320 | 90 |
| I-267 | 320 | 100 |
| I-27 | 320 | 90 |
| I-274 | 320 | 90 |
| I-275 | 320 | 90 |
| I-276 | 320 | 80 |
| I-278 | 320 | 80 |
| I-279 | 320 | 80 |
| I-28 | 320 | 90 |
| I-285 | 320 | 90 |
| I-287 | 320 | 90 |
| I-288 | 320 | 80 |
| I-289 | 320 | 90 |
| I-290 | 320 | 80 |
| I-291 | 320 | 80 |
| I-293 | 320 | 80 |
| I-294 | 320 | 90 |
| I-296 | 320 | 80 |
| I-298 | 320 | 90 |
| I-299 | 320 | 80 |
| I-300 | 320 | 90 |
| I-302 | 320 | 80 |
| I-304 | 320 | 90 |
| I-305 | 320 | 90 |
| I-306 | 320 | 90 |
| I-307 | 320 | 100 |
| I-308 | 320 | 100 |
| I-309 | 320 | 90 |
| I-310 | 320 | 90 |
| I-311 | 320 | 80 |
| I-312 | 320 | 90 |
| I-313 | 320 | 90 |
| I-314 | 320 | 90 |
| I-315 | 320 | 90 |
| I-316 | 320 | 100 |
| I-317 | 320 | 90 |
| I-318 | 320 | 90 |
| I-319 | 320 | 80 |
| I-320 | 320 | 90 |
| I-321 | 320 | 100 |
| I-322 | 320 | 80 |
| I-323 | 320 | 90 |
| I-324 | 320 | 90 |
| I-325 | 320 | 90 |
| I-326 | 320 | 90 |
| I-327 | 320 | 90 |
| I-328 | 320 | 90 |
| I-330 | 320 | 90 |
| I-331 | 320 | 90 |
| I-332 | 320 | 90 |
| I-333 | 320 | 90 |
| I-334 | 320 | 90 |
| I-335 | 320 | 90 |
| I-336 | 320 | 80 |
| I-338 | 320 | 90 |
| I-339 | 320 | 90 |
| I-340 | 320 | 90 |
| I-341 | 320 | 90 |
| I-342 | 320 | 90 |
| I-343 | 320 | 90 |
| I-344 | 320 | 90 |
| I-348 | 320 | 80 |
| I-350 | 320 | 90 |
| I-351 | 320 | 80 |
| I-352 | 320 | 80 |
| I-354 | 320 | 90 |
| I-358 | 320 | 90 |
| I-359 | 320 | 80 |
| I-360 | 320 | 90 |
| I-362 | 320 | 80 |
| I-366 | 320 | 90 |
| I-369 | 320 | 90 |
| I-370 | 320 | 90 |
| I-371 | 320 | 90 |
| I-375 | 320 | 90 |
| I-376 | 320 | 90 |
| I-380 | 320 | 90 |
| I-381 | 320 | 80 |
| I-382 | 320 | 90 |
| I-383 | 320 | 90 |
| I-388 | 320 | 80 |
| I-41 | 320 | 90 |
| I-42 | 320 | 100 |
| I-48 | 320 | 80 |
| I-58 | 320 | 80 |
| I-66 | 320 | 90 |
| I-69 | 320 | 80 |
| I-85 | 320 | 100 |
| I-86 | 320 | 100 |
| I-87 | 320 | 80 |
| I-88 | 320 | 80 |
| I-94 | 320 | 100 |
| I-95 | 320 | 80 |
| I-96 | 320 | 90 |
| III-22 | 320 | 100 |
| III-24 | 320 | 100 |
| III-29 | 320 | 80 |

TABLE B8a
Post-emergence action at 80 g/ha against MATIN in %

| Example number | Dosage [g/ha] | MATIN |
|---|---|---|
| I-02 | 80 | 80 |
| I-03 | 80 | 80 |
| I-07 | 80 | 90 |
| I-08 | 80 | 90 |
| I-100 | 80 | 90 |
| I-116 | 80 | 80 |
| I-118 | 80 | 80 |
| I-125 | 80 | 80 |
| I-126 | 80 | 80 |
| I-141 | 80 | 80 |
| I-144 | 80 | 90 |
| I-145 | 80 | 80 |
| I-146 | 80 | 90 |
| I-148 | 80 | 80 |
| I-149 | 80 | 80 |
| I-15 | 80 | 80 |
| I-151 | 80 | 80 |
| I-165 | 80 | 80 |
| I-166 | 80 | 80 |
| I-168 | 80 | 90 |
| I-169 | 80 | 80 |
| I-171 | 80 | 80 |
| I-172 | 80 | 90 |
| I-177 | 80 | 90 |
| I-179 | 80 | 80 |
| I-18 | 80 | 80 |
| I-181 | 80 | 80 |
| I-182 | 80 | 80 |
| I-183 | 80 | 80 |
| I-189 | 80 | 90 |
| I-197 | 80 | 80 |
| I-208 | 80 | 80 |
| I-210 | 80 | 80 |
| I-211 | 80 | 80 |
| I-216 | 80 | 80 |
| I-227 | 80 | 80 |
| I-231 | 80 | 80 |
| I-266 | 80 | 80 |
| I-275 | 80 | 80 |
| I-29 | 80 | 90 |
| I-30 | 80 | 90 |
| I-304 | 80 | 90 |
| I-308 | 80 | 80 |
| I-313 | 80 | 80 |
| I-316 | 80 | 80 |
| I-317 | 80 | 90 |
| I-318 | 80 | 80 |
| I-33 | 80 | 80 |
| I-331 | 80 | 80 |
| I-344 | 80 | 80 |
| I-35 | 80 | 90 |
| I-354 | 80 | 80 |
| I-370 | 80 | 80 |
| I-374 | 80 | 80 |
| I-42 | 80 | 80 |
| I-51 | 80 | 80 |
| I-59 | 80 | 80 |
| I-64 | 80 | 90 |
| I-66 | 80 | 80 |
| I-74 | 80 | 90 |
| I-75 | 80 | 90 |
| I-76 | 80 | 80 |
| I-77 | 80 | 90 |
| I-81 | 80 | 90 |
| I-85 | 80 | 80 |
| I-93 | 80 | 80 |
| I-97 | 80 | 80 |
| I-98 | 80 | 80 |
| III-29 | 80 | 80 |

TABLE B8b
Post-emergence action at 320 g/ha against MATIN in %

| Example number | Dosage [g/ha] | MATIN |
|---|---|---|
| I-01 | 320 | 90 |
| I-02 | 320 | 80 |
| I-03 | 320 | 90 |
| I-05 | 320 | 100 |
| I-07 | 320 | 100 |
| I-08 | 320 | 90 |
| I-130 | 320 | 80 |
| I-136 | 320 | 80 |
| I-141 | 320 | 90 |
| I-144 | 320 | 90 |
| I-145 | 320 | 90 |
| I-146 | 320 | 90 |
| I-148 | 320 | 80 |
| I-149 | 320 | 90 |
| I-15 | 320 | 90 |
| I-151 | 320 | 90 |
| I-153 | 320 | 80 |
| I-154 | 320 | 80 |
| I-155 | 320 | 80 |
| I-156 | 320 | 80 |
| I-159 | 320 | 80 |
| I-16 | 320 | 80 |
| I-164 | 320 | 80 |
| I-165 | 320 | 90 |
| I-166 | 320 | 90 |
| I-168 | 320 | 90 |
| I-169 | 320 | 90 |
| I-171 | 320 | 90 |
| I-172 | 320 | 90 |
| I-176 | 320 | 80 |
| I-177 | 320 | 90 |
| I-179 | 320 | 90 |
| I-18 | 320 | 80 |
| I-180 | 320 | 90 |
| I-181 | 320 | 80 |
| I-182 | 320 | 90 |
| I-183 | 320 | 80 |
| I-184 | 320 | 80 |
| I-185 | 320 | 80 |
| I-187 | 320 | 80 |
| I-188 | 320 | 80 |
| I-189 | 320 | 90 |
| I-190 | 320 | 80 |
| I-191 | 320 | 80 |
| I-192 | 320 | 80 |
| I-193 | 320 | 80 |
| I-196 | 320 | 80 |
| I-197 | 320 | 90 |
| I-198 | 320 | 80 |
| I-199 | 320 | 80 |
| I-204 | 320 | 80 |
| I-206 | 320 | 80 |
| I-207 | 320 | 80 |
| I-208 | 320 | 80 |
| I-210 | 320 | 80 |
| I-211 | 320 | 80 |
| I-212 | 320 | 80 |
| I-216 | 320 | 80 |
| I-217 | 320 | 80 |
| I-218 | 320 | 80 |
| I-227 | 320 | 90 |
| I-229 | 320 | 80 |
| I-231 | 320 | 80 |
| I-236 | 320 | 80 |
| I-239 | 320 | 80 |
| I-241 | 320 | 80 |
| I-243 | 320 | 80 |
| I-246 | 320 | 80 |
| I-251 | 320 | 90 |
| I-252 | 320 | 80 |
| I-264 | 320 | 80 |
| I-266 | 320 | 90 |
| I-273 | 320 | 80 |
| I-274 | 320 | 90 |
| I-275 | 320 | 90 |
| I-276 | 320 | 80 |
| I-28 | 320 | 80 |
| I-285 | 320 | 80 |
| I-287 | 320 | 80 |
| I-289 | 320 | 90 |
| I-293 | 320 | 80 |
| I-294 | 320 | 90 |
| I-298 | 320 | 80 |
| I-300 | 320 | 80 |
| I-304 | 320 | 90 |
| I-305 | 320 | 80 |
| I-307 | 320 | 80 |
| I-308 | 320 | 90 |
| I-309 | 320 | 80 |
| I-313 | 320 | 90 |
| I-314 | 320 | 80 |
| I-316 | 320 | 90 |
| I-317 | 320 | 90 |
| I-318 | 320 | 80 |
| I-321 | 320 | 80 |
| I-323 | 320 | 90 |
| I-324 | 320 | 80 |
| I-326 | 320 | 80 |
| I-331 | 320 | 80 |
| I-334 | 320 | 80 |
| I-338 | 320 | 90 |
| I-341 | 320 | 90 |
| I-342 | 320 | 100 |
| I-343 | 320 | 80 |
| I-344 | 320 | 80 |
| I-348 | 320 | 80 |
| I-354 | 320 | 80 |
| I-366 | 320 | 80 |
| I-369 | 320 | 80 |
| I-370 | 320 | 80 |
| I-373 | 320 | 90 |
| I-375 | 320 | 80 |
| I-383 | 320 | 80 |
| I-41 | 320 | 80 |
| I-42 | 320 | 90 |
| I-66 | 320 | 90 |
| I-69 | 320 | 80 |
| I-85 | 320 | 90 |
| I-86 | 320 | 80 |
| I-87 | 320 | 80 |
| I-88 | 320 | 80 |
| I-94 | 320 | 80 |
| I-95 | 320 | 80 |
| I-96 | 320 | 80 |
| II-41 | 320 | 80 |
| II-42 | 320 | 80 |
| II-43 | 320 | 80 |
| II-45 | 320 | 90 |
| III-29 | 320 | 90 |

TABLE B9a
Post-emergence action at 80 g/ha against PHBPU in %

| Example number | Dosage [g/ha] | PHBPU |
|---|---|---|
| I-01 | 80 | 80 |
| I-02 | 80 | 90 |
| I-03 | 80 | 90 |
| I-07 | 80 | 80 |
| I-08 | 80 | 80 |
| I-100 | 80 | 90 |
| I-103 | 80 | 80 |
| I-118 | 80 | 80 |
| I-119 | 80 | 80 |
| I-120 | 80 | 80 |
| I-125 | 80 | 80 |
| I-141 | 80 | 80 |
| I-144 | 80 | 90 |
| I-145 | 80 | 90 |
| I-146 | 80 | 90 |
| I-148 | 80 | 80 |
| I-149 | 80 | 80 |
| I-151 | 80 | 90 |
| I-152 | 80 | 80 |
| I-153 | 80 | 80 |
| I-154 | 80 | 80 |
| I-155 | 80 | 80 |
| I-159 | 80 | 80 |
| I-16 | 80 | 90 |
| I-163 | 80 | 80 |
| I-164 | 80 | 80 |
| I-165 | 80 | 80 |
| I-166 | 80 | 80 |
| I-167 | 80 | 90 |
| I-168 | 80 | 90 |
| I-169 | 80 | 80 |
| I-171 | 80 | 80 |
| I-172 | 80 | 80 |
| I-174 | 80 | 80 |
| I-175 | 80 | 80 |
| I-177 | 80 | 90 |
| I-179 | 80 | 80 |
| I-181 | 80 | 90 |
| I-182 | 80 | 90 |
| I-183 | 80 | 80 |
| I-184 | 80 | 90 |
| I-188 | 80 | 90 |
| I-189 | 80 | 90 |
| I-19 | 80 | 90 |
| I-191 | 80 | 90 |
| I-192 | 80 | 90 |
| I-193 | 80 | 90 |
| I-194 | 80 | 90 |
| I-195 | 80 | 90 |
| I-196 | 80 | 90 |
| I-197 | 80 | 90 |
| I-198 | 80 | 80 |
| I-20 | 80 | 90 |
| I-207 | 80 | 80 |
| I-208 | 80 | 80 |
| I-210 | 80 | 90 |
| I-211 | 80 | 90 |
| I-212 | 80 | 90 |
| I-216 | 80 | 80 |
| I-218 | 80 | 90 |
| I-221 | 80 | 90 |
| I-223 | 80 | 80 |
| I-225 | 80 | 80 |
| I-227 | 80 | 90 |
| I-235 | 80 | 80 |
| I-237 | 80 | 80 |
| I-239 | 80 | 80 |
| I-241 | 80 | 80 |
| I-242 | 80 | 80 |
| I-245 | 80 | 80 |
| I-246 | 80 | 80 |
| I-247 | 80 | 90 |
| I-264 | 80 | 80 |
| I-266 | 80 | 80 |
| I-273 | 80 | 80 |
| I-275 | 80 | 90 |
| I-276 | 80 | 80 |
| I-278 | 80 | 80 |
| I-279 | 80 | 80 |
| I-28 | 80 | 80 |
| I-285 | 80 | 80 |
| I-288 | 80 | 80 |
| I-289 | 80 | 80 |
| I-291 | 80 | 80 |
| I-304 | 80 | 80 |
| I-307 | 80 | 80 |
| I-309 | 80 | 80 |
| I-312 | 80 | 80 |
| I-313 | 80 | 90 |
| I-314 | 80 | 90 |
| I-315 | 80 | 80 |
| I-316 | 80 | 90 |
| I-318 | 80 | 90 |
| I-319 | 80 | 80 |
| I-32 | 80 | 80 |
| I-322 | 80 | 80 |
| I-326 | 80 | 80 |
| I-328 | 80 | 90 |
| I-330 | 80 | 80 |
| I-332 | 80 | 80 |
| I-333 | 80 | 80 |
| I-336 | 80 | 90 |
| I-337 | 80 | 90 |
| I-34 | 80 | 80 |
| I-340 | 80 | 90 |
| I-341 | 80 | 90 |
| I-342 | 80 | 80 |
| I-344 | 80 | 90 |
| I-348 | 80 | 80 |
| I-352 | 80 | 90 |
| I-357 | 80 | 80 |
| I-36 | 80 | 80 |
| I-360 | 80 | 80 |
| I-366 | 80 | 80 |
| I-369 | 80 | 80 |
| I-37 | 80 | 80 |
| I-370 | 80 | 80 |
| I-375 | 80 | 80 |
| I-38 | 80 | 90 |
| I-382 | 80 | 80 |
| I-388 | 80 | 80 |
| I-39 | 80 | 80 |
| I-40 | 80 | 80 |
| I-46 | 80 | 80 |
| I-51 | 80 | 90 |
| I-56 | 80 | 80 |
| I-59 | 80 | 80 |
| I-64 | 80 | 90 |
| I-69 | 80 | 80 |
| I-73 | 80 | 90 |
| I-74 | 80 | 90 |
| I-75 | 80 | 90 |
| I-76 | 80 | 90 |
| I-77 | 80 | 90 |
| I-79 | 80 | 80 |
| I-80 | 80 | 80 |
| I-81 | 80 | 90 |
| I-83 | 80 | 90 |
| I-84 | 80 | 90 |
| I-85 | 80 | 90 |
| I-88 | 80 | 80 |
| I-91 | 80 | 80 |
| I-96 | 80 | 90 |
| I-97 | 80 | 80 |
| I-99 | 80 | 80 |
| II-41 | 80 | 80 |
| II-43 | 80 | 80 |
| III-17 | 80 | 80 |
| III-19 | 80 | 80 |
| III-29 | 80 | 80 |

TABLE B9b
Post-emergence action at 320 g/ha against PHBPU in %

| Example number | Dosage [g/ha] | PHBPU |
|---|---|---|
| I-01 | 320 | 80 |
| I-02 | 320 | 100 |
| I-03 | 320 | 100 |
| I-04 | 320 | 100 |
| I-05 | 320 | 90 |
| I-07 | 320 | 80 |
| I-08 | 320 | 80 |
| I-11 | 320 | 90 |
| I-130 | 320 | 80 |
| I-133 | 320 | 80 |
| I-136 | 320 | 90 |
| I-138 | 320 | 80 |
| I-139 | 320 | 80 |
| I-141 | 320 | 90 |
| I-144 | 320 | 90 |
| I-145 | 320 | 90 |
| I-146 | 320 | 90 |
| I-147 | 320 | 90 |
| I-148 | 320 | 90 |
| I-149 | 320 | 80 |
| I-15 | 320 | 90 |
| I-151 | 320 | 90 |
| I-152 | 320 | 90 |
| I-153 | 320 | 90 |
| I-154 | 320 | 90 |
| I-155 | 320 | 90 |
| I-156 | 320 | 90 |
| I-157 | 320 | 80 |
| I-159 | 320 | 80 |
| I-16 | 320 | 90 |
| I-160 | 320 | 90 |
| I-162 | 320 | 90 |
| I-163 | 320 | 90 |
| I-164 | 320 | 90 |
| I-165 | 320 | 90 |
| I-166 | 320 | 90 |
| I-167 | 320 | 90 |
| I-168 | 320 | 90 |
| I-169 | 320 | 90 |
| I-17 | 320 | 80 |
| I-171 | 320 | 90 |
| I-172 | 320 | 90 |
| I-174 | 320 | 90 |
| I-175 | 320 | 90 |
| I-177 | 320 | 90 |
| I-179 | 320 | 90 |
| I-181 | 320 | 100 |
| I-182 | 320 | 90 |
| I-183 | 320 | 90 |
| I-184 | 320 | 90 |
| I-187 | 320 | 80 |
| I-188 | 320 | 90 |
| I-189 | 320 | 90 |
| I-191 | 320 | 100 |
| I-192 | 320 | 90 |
| I-193 | 320 | 100 |
| I-194 | 320 | 90 |
| I-195 | 320 | 90 |
| I-196 | 320 | 90 |
| I-197 | 320 | 100 |
| I-198 | 320 | 90 |
| I-199 | 320 | 80 |
| I-20 | 320 | 90 |
| I-202 | 320 | 90 |
| I-204 | 320 | 80 |
| I-206 | 320 | 90 |
| I-207 | 320 | 90 |
| I-208 | 320 | 90 |
| I-210 | 320 | 100 |
| I-211 | 320 | 100 |
| I-212 | 320 | 100 |
| I-216 | 320 | 80 |
| I-218 | 320 | 100 |
| I-221 | 320 | 100 |
| I-223 | 320 | 80 |
| I-225 | 320 | 80 |
| I-227 | 320 | 100 |
| I-229 | 320 | 80 |
| I-230 | 320 | 90 |
| I-231 | 320 | 80 |
| I-235 | 320 | 80 |
| I-236 | 320 | 80 |
| I-237 | 320 | 90 |
| I-238 | 320 | 80 |
| I-239 | 320 | 90 |
| I-241 | 320 | 90 |
| I-242 | 320 | 80 |
| I-244 | 320 | 80 |
| I-245 | 320 | 90 |
| I-246 | 320 | 80 |
| I-247 | 320 | 90 |
| I-248 | 320 | 90 |
| I-252 | 320 | 90 |
| I-260 | 320 | 80 |
| I-261 | 320 | 80 |
| I-264 | 320 | 80 |
| I-266 | 320 | 90 |
| I-267 | 320 | 90 |
| I-268 | 320 | 80 |
| I-273 | 320 | 90 |
| I-274 | 320 | 80 |
| I-275 | 320 | 100 |
| I-276 | 320 | 90 |
| I-277 | 320 | 80 |
| I-278 | 320 | 80 |
| I-279 | 320 | 80 |
| I-28 | 320 | 80 |
| I-285 | 320 | 90 |
| I-287 | 320 | 80 |
| I-288 | 320 | 90 |
| I-289 | 320 | 80 |
| I-290 | 320 | 90 |
| I-291 | 320 | 90 |
| I-293 | 320 | 80 |
| I-296 | 320 | 90 |
| I-298 | 320 | 90 |
| I-299 | 320 | 90 |
| I-300 | 320 | 80 |
| I-302 | 320 | 80 |
| I-304 | 320 | 90 |
| I-305 | 320 | 90 |
| I-307 | 320 | 90 |
| I-308 | 320 | 90 |
| I-309 | 320 | 90 |
| I-311 | 320 | 90 |
| I-312 | 320 | 90 |
| I-313 | 320 | 90 |
| I-314 | 320 | 90 |
| I-315 | 320 | 80 |
| I-316 | 320 | 90 |
| I-317 | 320 | 80 |
| I-318 | 320 | 90 |
| I-319 | 320 | 90 |
| I-320 | 320 | 80 |
| I-321 | 320 | 90 |
| I-322 | 320 | 80 |
| I-323 | 320 | 80 |
| I-324 | 320 | 90 |
| I-325 | 320 | 80 |
| I-326 | 320 | 90 |
| I-328 | 320 | 90 |
| I-329 | 320 | 80 |
| I-330 | 320 | 90 |
| I-331 | 320 | 90 |
| I-332 | 320 | 80 |
| I-333 | 320 | 90 |
| I-334 | 320 | 80 |
| I-336 | 320 | 100 |
| I-337 | 320 | 90 |
| I-339 | 320 | 80 |
| I-340 | 320 | 90 |
| I-341 | 320 | 100 |
| I-342 | 320 | 90 |
| I-343 | 320 | 90 |
| I-344 | 320 | 90 |
| I-346 | 320 | 90 |
| I-348 | 320 | 80 |
| I-351 | 320 | 80 |
| I-352 | 320 | 90 |
| I-354 | 320 | 90 |
| I-357 | 320 | 80 |
| I-359 | 320 | 80 |
| I-360 | 320 | 80 |
| I-362 | 320 | 80 |
| I-363 | 320 | 80 |
| I-366 | 320 | 80 |
| I-369 | 320 | 90 |
| I-370 | 320 | 90 |
| I-371 | 320 | 80 |
| I-373 | 320 | 80 |
| I-375 | 320 | 90 |
| I-376 | 320 | 80 |
| I-377 | 320 | 90 |
| I-382 | 320 | 80 |
| I-388 | 320 | 100 |
| I-41 | 320 | 90 |
| I-42 | 320 | 90 |
| I-48 | 320 | 90 |
| I-58 | 320 | 80 |
| I-66 | 320 | 80 |
| I-69 | 320 | 80 |
| I-80 | 320 | 80 |
| I-85 | 320 | 90 |
| I-87 | 320 | 90 |
| I-88 | 320 | 80 |
| I-94 | 320 | 80 |
| I-96 | 320 | 90 |
| II-38 | 320 | 80 |
| II-39 | 320 | 90 |
| II-40 | 320 | 80 |
| II-41 | 320 | 90 |
| II-42 | 320 | 90 |
| II-43 | 320 | 90 |
| II-45 | 320 | 90 |
| III-22 | 320 | 80 |
| III-23 | 320 | 80 |
| III-29 | 320 | 90 |

TABLE 10a
Post-emergence action at 80 g/ha against POLCO in %

| Example number | Dosage [g/ha] | POLCO |
|---|---|---|
| I-02 | 80 | 80 |
| I-03 | 80 | 90 |
| I-05 | 80 | 90 |
| I-07 | 80 | 80 |
| I-08 | 80 | 90 |
| I-10 | 80 | 80 |
| I-100 | 80 | 90 |
| I-102 | 80 | 90 |
| I-121 | 80 | 80 |
| I-122 | 80 | 90 |
| I-133 | 80 | 80 |
| I-137 | 80 | 80 |
| I-141 | 80 | 80 |
| I-144 | 80 | 80 |
| I-145 | 80 | 90 |
| I-146 | 80 | 80 |
| I-165 | 80 | 80 |
| I-166 | 80 | 80 |
| I-167 | 80 | 80 |
| I-169 | 80 | 80 |
| I-171 | 80 | 80 |
| I-175 | 80 | 80 |
| I-176 | 80 | 80 |
| I-177 | 80 | 90 |
| I-179 | 80 | 90 |
| I-180 | 80 | 80 |
| I-181 | 80 | 80 |
| I-182 | 80 | 80 |
| I-184 | 80 | 80 |
| I-187 | 80 | 80 |
| I-188 | 80 | 80 |
| I-19 | 80 | 90 |
| I-190 | 80 | 80 |
| I-193 | 80 | 80 |
| I-196 | 80 | 80 |
| I-197 | 80 | 90 |
| I-212 | 80 | 80 |
| I-217 | 80 | 80 |
| I-223 | 80 | 80 |
| I-227 | 80 | 90 |
| I-239 | 80 | 80 |
| I-241 | 80 | 80 |
| I-242 | 80 | 80 |
| I-244 | 80 | 80 |
| I-247 | 80 | 80 |
| I-26 | 80 | 80 |
| I-260 | 80 | 80 |
| I-266 | 80 | 80 |
| I-267 | 80 | 80 |
| I-27 | 80 | 80 |
| I-275 | 80 | 80 |
| I-291 | 80 | 80 |
| I-293 | 80 | 80 |
| I-307 | 80 | 80 |
| I-310 | 80 | 80 |
| I-312 | 80 | 80 |
| I-315 | 80 | 80 |
| I-318 | 80 | 90 |
| I-321 | 80 | 80 |
| I-322 | 80 | 80 |
| I-324 | 80 | 80 |
| I-326 | 80 | 80 |
| I-328 | 80 | 90 |
| I-331 | 80 | 80 |
| I-335 | 80 | 80 |
| I-337 | 80 | 80 |
| I-338 | 80 | 80 |
| I-339 | 80 | 90 |
| I-341 | 80 | 80 |
| I-342 | 80 | 80 |
| I-343 | 80 | 80 |
| I-344 | 80 | 80 |
| I-350 | 80 | 80 |
| I-358 | 80 | 90 |
| I-36 | 80 | 80 |
| I-37 | 80 | 80 |
| I-375 | 80 | 80 |
| I-38 | 80 | 80 |
| I-39 | 80 | 90 |
| I-41 | 80 | 80 |
| I-42 | 80 | 80 |
| I-44 | 80 | 90 |
| I-48 | 80 | 80 |
| I-56 | 80 | 80 |
| I-59 | 80 | 80 |
| I-66 | 80 | 80 |
| I-73 | 80 | 90 |
| I-74 | 80 | 90 |
| I-75 | 80 | 90 |
| I-76 | 80 | 80 |
| I-77 | 80 | 80 |
| I-79 | 80 | 80 |
| I-80 | 80 | 80 |
| I-81 | 80 | 80 |
| I-84 | 80 | 90 |
| I-85 | 80 | 90 |
| I-87 | 80 | 80 |
| I-88 | 80 | 80 |
| I-94 | 80 | 80 |
| I-95 | 80 | 80 |
| I-96 | 80 | 80 |
| I-99 | 80 | 90 |
| II-40 | 80 | 80 |
| II-45 | 80 | 80 |

TABLE 10b
Post-emergence action at 320 g/ha against POLCO in %

| Example number | Dosage [g/ha] | POLCO |
|---|---|---|
| I-02 | 320 | 80 |
| I-03 | 320 | 90 |
| I-04 | 320 | 90 |
| I-05 | 320 | 90 |
| I-07 | 320 | 80 |
| I-08 | 320 | 90 |
| I-10 | 320 | 80 |
| I-111 | 320 | 80 |
| I-130 | 320 | 80 |
| I-133 | 320 | 80 |
| I-137 | 320 | 80 |
| I-138 | 320 | 80 |
| I-139 | 320 | 90 |
| I-140 | 320 | 90 |
| I-141 | 320 | 80 |
| I-144 | 320 | 80 |
| I-145 | 320 | 90 |
| I-146 | 320 | 90 |
| I-147 | 320 | 90 |
| I-149 | 320 | 80 |
| I-150 | 320 | 90 |
| I-151 | 320 | 80 |
| I-152 | 320 | 80 |
| I-153 | 320 | 80 |
| I-154 | 320 | 80 |
| I-156 | 320 | 80 |
| I-165 | 320 | 90 |
| I-166 | 320 | 80 |
| I-167 | 320 | 80 |
| I-168 | 320 | 80 |
| I-169 | 320 | 80 |
| I-171 | 320 | 90 |
| I-172 | 320 | 80 |
| I-174 | 320 | 80 |
| I-175 | 320 | 80 |
| I-176 | 320 | 80 |
| I-177 | 320 | 90 |
| I-179 | 320 | 90 |
| I-180 | 320 | 90 |
| I-181 | 320 | 80 |
| I-182 | 320 | 90 |
| I-183 | 320 | 90 |
| I-184 | 320 | 80 |
| I-185 | 320 | 80 |
| I-186 | 320 | 80 |
| I-187 | 320 | 80 |
| I-188 | 320 | 80 |
| I-190 | 320 | 80 |
| I-192 | 320 | 80 |
| I-193 | 320 | 80 |
| I-195 | 320 | 80 |
| I-196 | 320 | 80 |
| I-197 | 320 | 90 |
| I-20 | 320 | 80 |
| I-202 | 320 | 80 |
| I-208 | 320 | 80 |
| I-212 | 320 | 90 |
| I-217 | 320 | 80 |
| I-218 | 320 | 80 |
| I-223 | 320 | 80 |
| I-227 | 320 | 90 |
| I-229 | 320 | 80 |
| I-230 | 320 | 80 |
| I-235 | 320 | 80 |
| I-236 | 320 | 80 |
| I-237 | 320 | 80 |
| I-238 | 320 | 80 |
| I-239 | 320 | 80 |
| I-240 | 320 | 80 |
| I-241 | 320 | 80 |
| I-242 | 320 | 80 |
| I-244 | 320 | 80 |
| I-245 | 320 | 80 |
| I-246 | 320 | 80 |
| I-247 | 320 | 80 |
| I-252 | 320 | 80 |
| I-26 | 320 | 80 |
| I-260 | 320 | 80 |
| I-261 | 320 | 80 |
| I-266 | 320 | 90 |
| I-267 | 320 | 80 |
| I-27 | 320 | 80 |
| I-272 | 320 | 80 |
| I-273 | 320 | 80 |
| I-274 | 320 | 80 |
| I-275 | 320 | 80 |
| I-28 | 320 | 80 |
| I-285 | 320 | 80 |
| I-288 | 320 | 80 |
| I-289 | 320 | 80 |
| I-290 | 320 | 80 |
| I-291 | 320 | 80 |
| I-293 | 320 | 80 |
| I-296 | 320 | 80 |
| I-298 | 320 | 80 |
| I-299 | 320 | 80 |
| I-302 | 320 | 80 |
| I-305 | 320 | 80 |
| I-306 | 320 | 80 |
| I-307 | 320 | 80 |
| I-309 | 320 | 80 |
| I-31 | 320 | 80 |
| I-310 | 320 | 80 |
| I-311 | 320 | 80 |
| I-312 | 320 | 80 |
| I-314 | 320 | 90 |
| I-315 | 320 | 80 |
| I-318 | 320 | 90 |
| I-319 | 320 | 80 |
| I-320 | 320 | 80 |
| I-321 | 320 | 90 |
| I-322 | 320 | 80 |
| I-324 | 320 | 80 |
| I-325 | 320 | 80 |
| I-326 | 320 | 90 |
| I-327 | 320 | 80 |
| I-328 | 320 | 90 |
| I-331 | 320 | 80 |
| I-333 | 320 | 80 |
| I-334 | 320 | 80 |
| I-335 | 320 | 80 |
| I-336 | 320 | 80 |
| I-337 | 320 | 80 |
| I-338 | 320 | 80 |
| I-339 | 320 | 90 |
| I-340 | 320 | 90 |
| I-341 | 320 | 80 |
| I-342 | 320 | 90 |
| I-343 | 320 | 80 |
| I-344 | 320 | 90 |
| I-347 | 320 | 80 |
| I-348 | 320 | 80 |
| I-350 | 320 | 90 |
| I-351 | 320 | 80 |
| I-354 | 320 | 80 |
| I-358 | 320 | 90 |
| I-359 | 320 | 80 |
| I-366 | 320 | 80 |
| I-369 | 320 | 80 |
| I-373 | 320 | 80 |
| I-375 | 320 | 90 |
| I-376 | 320 | 80 |
| I-377 | 320 | 80 |
| I-380 | 320 | 80 |
| I-382 | 320 | 80 |
| I-41 | 320 | 80 |
| I-42 | 320 | 90 |
| I-48 | 320 | 90 |
| I-60 | 320 | 80 |
| I-65 | 320 | 80 |
| I-66 | 320 | 90 |
| I-80 | 320 | 80 |
| I-85 | 320 | 90 |
| I-86 | 320 | 90 |
| I-87 | 320 | 80 |
| I-88 | 320 | 80 |
| I-94 | 320 | 90 |
| I-95 | 320 | 80 |
| I-96 | 320 | 80 |
| II-38 | 320 | 90 |
| II-40 | 320 | 80 |
| II-42 | 320 | 80 |
| II-43 | 320 | 80 |
| II-44 | 320 | 90 |
| II-45 | 320 | 90 |
| II-46 | 320 | 80 |
| III-08 | 320 | 80 |
| III-24 | 320 | 90 |
| III-29 | 320 | 80 |
| III-30 | 320 | 90 |

TABLE 11a
Post-emergence action at 80 g/ha against SETVI in %

| Example number | Dosage [g/ha] | SETVI |
|---|---|---|
| I-02 | 80 | 80 |
| I-05 | 80 | 80 |
| I-07 | 80 | 90 |
| I-08 | 80 | 80 |
| I-10 | 80 | 80 |
| I-100 | 80 | 80 |
| I-107 | 80 | 80 |
| I-11 | 80 | 80 |
| I-114 | 80 | 80 |
| I-116 | 80 | 80 |
| I-118 | 80 | 80 |
| I-119 | 80 | 80 |
| I-125 | 80 | 80 |
| I-126 | 80 | 80 |
| I-13 | 80 | 80 |
| I-141 | 80 | 90 |
| I-144 | 80 | 90 |
| I-145 | 80 | 90 |
| I-146 | 80 | 90 |
| I-148 | 80 | 80 |
| I-149 | 80 | 80 |
| I-15 | 80 | 80 |
| I-151 | 80 | 90 |
| I-152 | 80 | 80 |
| I-155 | 80 | 80 |
| I-16 | 80 | 80 |
| I-163 | 80 | 80 |
| I-164 | 80 | 80 |
| I-165 | 80 | 90 |
| I-166 | 80 | 90 |
| I-168 | 80 | 80 |
| I-17 | 80 | 80 |
| I-171 | 80 | 90 |
| I-172 | 80 | 80 |
| I-176 | 80 | 80 |
| I-177 | 80 | 90 |
| I-179 | 80 | 90 |
| I-18 | 80 | 80 |
| I-180 | 80 | 80 |
| I-181 | 80 | 80 |
| I-182 | 80 | 80 |
| I-183 | 80 | 90 |
| I-184 | 80 | 80 |
| I-188 | 80 | 80 |
| I-189 | 80 | 90 |
| I-19 | 80 | 80 |
| I-190 | 80 | 80 |
| I-191 | 80 | 80 |
| I-192 | 80 | 80 |
| I-194 | 80 | 90 |
| I-195 | 80 | 90 |
| I-196 | 80 | 80 |
| I-197 | 80 | 90 |
| I-198 | 80 | 80 |
| I-199 | 80 | 80 |
| I-204 | 80 | 90 |
| I-207 | 80 | 90 |
| I-208 | 80 | 80 |
| I-210 | 80 | 80 |
| I-211 | 80 | 80 |
| I-212 | 80 | 80 |
| I-217 | 80 | 80 |
| I-218 | 80 | 80 |
| I-221 | 80 | 90 |
| I-227 | 80 | 90 |
| I-229 | 80 | 80 |
| I-252 | 80 | 80 |
| I-264 | 80 | 90 |
| I-267 | 80 | 90 |
| I-274 | 80 | 90 |
| I-275 | 80 | 90 |
| I-279 | 80 | 80 |
| I-28 | 80 | 80 |
| I-285 | 80 | 90 |
| I-287 | 80 | 90 |
| I-288 | 80 | 90 |
| I-289 | 80 | 90 |
| I-29 | 80 | 80 |
| I-290 | 80 | 90 |
| I-291 | 80 | 80 |
| I-294 | 80 | 90 |
| I-298 | 80 | 90 |
| I-302 | 80 | 80 |
| I-304 | 80 | 90 |
| I-307 | 80 | 90 |
| I-313 | 80 | 90 |
| I-314 | 80 | 90 |
| I-316 | 80 | 90 |
| I-318 | 80 | 90 |
| I-321 | 80 | 90 |
| I-322 | 80 | 80 |
| I-324 | 80 | 80 |
| I-326 | 80 | 90 |
| I-328 | 80 | 90 |
| I-331 | 80 | 90 |
| I-338 | 80 | 90 |
| I-339 | 80 | 90 |
| I-340 | 80 | 80 |
| I-341 | 80 | 80 |
| I-342 | 80 | 90 |
| I-343 | 80 | 90 |
| I-344 | 80 | 90 |
| I-348 | 80 | 80 |
| I-35 | 80 | 100 |
| I-350 | 80 | 80 |
| I-354 | 80 | 80 |
| I-36 | 80 | 80 |
| I-360 | 80 | 80 |
| I-366 | 80 | 90 |
| I-37 | 80 | 80 |
| I-370 | 80 | 80 |
| I-374 | 80 | 90 |
| I-375 | 80 | 90 |
| I-38 | 80 | 80 |
| I-380 | 80 | 80 |
| I-382 | 80 | 90 |
| I-383 | 80 | 80 |
| I-388 | 80 | 90 |
| I-39 | 80 | 80 |
| I-40 | 80 | 80 |
| I-42 | 80 | 90 |
| I-43 | 80 | 90 |
| I-47 | 80 | 90 |
| I-48 | 80 | 80 |
| I-51 | 80 | 90 |
| I-52 | 80 | 80 |
| I-53 | 80 | 80 |
| I-54 | 80 | 80 |
| I-64 | 80 | 90 |
| I-66 | 80 | 80 |
| I-69 | 80 | 80 |
| I-73 | 80 | 80 |
| I-74 | 80 | 80 |
| I-75 | 80 | 80 |
| I-76 | 80 | 90 |
| I-77 | 80 | 80 |
| I-79 | 80 | 80 |
| I-81 | 80 | 80 |
| I-83 | 80 | 90 |
| I-84 | 80 | 80 |
| I-85 | 80 | 90 |
| I-86 | 80 | 100 |
| I-87 | 80 | 80 |
| I-91 | 80 | 80 |
| I-94 | 80 | 80 |
| I-96 | 80 | 90 |
| I-97 | 80 | 80 |
| III-22 | 80 | 100 |
| III-23 | 80 | 80 |
| III-24 | 80 | 100 |

TABLE B11b
Post-emergence action at 320 g/ha against SETVI in %

| Example number | Dosage [g/ha] | SETVI |
|---|---|---|
| I-01 | 320 | 80 |
| I-02 | 320 | 80 |
| I-03 | 320 | 90 |
| I-04 | 320 | 100 |
| I-05 | 320 | 90 |
| I-07 | 320 | 90 |
| I-08 | 320 | 90 |
| I-10 | 320 | 80 |
| I-11 | 320 | 90 |
| I-13 | 320 | 90 |
| I-133 | 320 | 80 |
| I-134 | 320 | 80 |
| I-136 | 320 | 90 |
| I-138 | 320 | 80 |
| I-141 | 320 | 90 |
| I-144 | 320 | 90 |
| I-145 | 320 | 90 |
| I-146 | 320 | 90 |
| I-148 | 320 | 80 |
| I-149 | 320 | 80 |
| I-15 | 320 | 90 |
| I-150 | 320 | 80 |
| I-151 | 320 | 90 |
| I-152 | 320 | 80 |
| I-153 | 320 | 80 |
| I-154 | 320 | 80 |
| I-155 | 320 | 90 |
| I-156 | 320 | 80 |
| I-157 | 320 | 80 |
| I-159 | 320 | 80 |
| I-16 | 320 | 80 |
| I-160 | 320 | 80 |
| I-162 | 320 | 80 |
| I-163 | 320 | 90 |
| I-164 | 320 | 80 |
| I-165 | 320 | 90 |
| I-166 | 320 | 90 |
| I-167 | 320 | 80 |
| I-168 | 320 | 90 |
| I-169 | 320 | 80 |
| I-17 | 320 | 80 |
| I-171 | 320 | 90 |
| I-172 | 320 | 80 |
| I-175 | 320 | 80 |
| I-176 | 320 | 80 |
| I-177 | 320 | 90 |
| I-179 | 320 | 90 |
| I-18 | 320 | 80 |
| I-180 | 320 | 90 |
| I-181 | 320 | 90 |
| I-182 | 320 | 80 |
| I-183 | 320 | 90 |
| I-184 | 320 | 90 |
| I-185 | 320 | 80 |
| I-187 | 320 | 80 |
| I-188 | 320 | 80 |
| I-189 | 320 | 90 |
| I-190 | 320 | 80 |
| I-191 | 320 | 80 |
| I-192 | 320 | 80 |
| I-193 | 320 | 90 |
| I-194 | 320 | 90 |
| I-195 | 320 | 90 |
| I-196 | 320 | 80 |
| I-197 | 320 | 90 |
| I-198 | 320 | 80 |
| I-199 | 320 | 90 |
| I-20 | 320 | 90 |
| I-202 | 320 | 80 |
| I-204 | 320 | 90 |
| I-207 | 320 | 90 |
| I-208 | 320 | 80 |
| I-210 | 320 | 80 |
| I-211 | 320 | 80 |
| I-212 | 320 | 80 |
| I-216 | 320 | 80 |
| I-217 | 320 | 90 |
| I-218 | 320 | 80 |
| I-221 | 320 | 90 |
| I-223 | 320 | 80 |
| I-225 | 320 | 80 |
| I-227 | 320 | 90 |
| I-229 | 320 | 80 |
| I-230 | 320 | 80 |
| I-231 | 320 | 80 |
| I-247 | 320 | 80 |
| I-251 | 320 | 90 |
| I-252 | 320 | 80 |
| I-26 | 320 | 90 |
| I-260 | 320 | 90 |
| I-264 | 320 | 90 |
| I-266 | 320 | 80 |
| I-267 | 320 | 90 |
| I-27 | 320 | 90 |
| I-272 | 320 | 80 |
| I-274 | 320 | 90 |
| I-275 | 320 | 90 |
| I-276 | 320 | 90 |
| I-278 | 320 | 90 |
| I-279 | 320 | 80 |
| I-28 | 320 | 90 |
| I-285 | 320 | 90 |
| I-286 | 320 | 80 |
| I-287 | 320 | 90 |
| I-288 | 320 | 90 |
| I-289 | 320 | 90 |
| I-290 | 320 | 90 |
| I-291 | 320 | 80 |
| I-293 | 320 | 80 |
| I-294 | 320 | 90 |
| I-296 | 320 | 80 |
| I-298 | 320 | 90 |
| I-299 | 320 | 90 |
| I-300 | 320 | 80 |
| I-302 | 320 | 90 |
| I-304 | 320 | 90 |
| I-305 | 320 | 90 |
| I-306 | 320 | 90 |
| I-307 | 320 | 90 |
| I-309 | 320 | 90 |
| I-310 | 320 | 90 |
| I-311 | 320 | 80 |
| I-312 | 320 | 80 |
| I-313 | 320 | 90 |
| I-314 | 320 | 90 |
| I-315 | 320 | 80 |
| I-316 | 320 | 90 |
| I-318 | 320 | 90 |
| I-319 | 320 | 80 |
| I-320 | 320 | 90 |
| I-321 | 320 | 90 |
| I-322 | 320 | 80 |
| I-323 | 320 | 90 |
| I-324 | 320 | 90 |
| I-326 | 320 | 90 |
| I-327 | 320 | 90 |
| I-328 | 320 | 90 |
| I-330 | 320 | 80 |
| I-331 | 320 | 90 |
| I-332 | 320 | 80 |
| I-333 | 320 | 80 |
| I-335 | 320 | 90 |
| I-336 | 320 | 80 |
| I-337 | 320 | 80 |
| I-338 | 320 | 90 |
| I-339 | 320 | 90 |
| I-340 | 320 | 90 |
| I-341 | 320 | 80 |
| I-342 | 320 | 90 |
| I-343 | 320 | 90 |
| I-344 | 320 | 90 |
| I-348 | 320 | 90 |
| I-350 | 320 | 90 |
| I-351 | 320 | 80 |
| I-352 | 320 | 80 |
| I-354 | 320 | 80 |
| I-357 | 320 | 80 |
| I-358 | 320 | 90 |
| I-360 | 320 | 80 |
| I-362 | 320 | 80 |
| I-366 | 320 | 90 |
| I-370 | 320 | 80 |
| I-371 | 320 | 80 |
| I-373 | 320 | 90 |
| I-375 | 320 | 90 |
| I-376 | 320 | 80 |
| I-377 | 320 | 90 |
| I-378 | 320 | 80 |
| I-380 | 320 | 90 |
| I-382 | 320 | 90 |
| I-383 | 320 | 80 |
| I-388 | 320 | 90 |
| I-41 | 320 | 80 |
| I-42 | 320 | 90 |
| I-48 | 320 | 80 |
| I-65 | 320 | 80 |
| I-66 | 320 | 90 |
| I-69 | 320 | 80 |
| I-80 | 320 | 80 |
| I-85 | 320 | 100 |
| I-86 | 320 | 100 |
| I-87 | 320 | 80 |
| I-88 | 320 | 80 |
| I-94 | 320 | 90 |
| I-95 | 320 | 90 |
| I-96 | 320 | 90 |
| II-37 | 320 | 80 |
| II-38 | 320 | 90 |
| II-39 | 320 | 90 |
| II-40 | 320 | 90 |
| II-41 | 320 | 90 |
| II-42 | 320 | 90 |
| II-43 | 320 | 90 |
| II-44 | 320 | 90 |
| II-45 | 320 | 90 |
| III-22 | 320 | 100 |
| III-23 | 320 | 100 |
| III-24 | 320 | 100 |
| III-29 | 320 | 90 |

TABLE B12a
Post-emergence action at 80 g/ha against VERPE in %

| Example number | Dosage [g/ha] | VERPE |
|---|---|---|
| I-01 | 80 | 80 |
| I-02 | 80 | 80 |
| I-03 | 80 | 80 |
| I-05 | 80 | 90 |
| I-07 | 80 | 90 |
| I-08 | 80 | 90 |
| I-100 | 80 | 80 |
| I-103 | 80 | 80 |
| I-116 | 80 | 80 |
| I-118 | 80 | 90 |
| I-119 | 80 | 80 |
| I-120 | 80 | 90 |
| I-121 | 80 | 80 |
| I-122 | 80 | 80 |
| I-130 | 80 | 80 |
| I-133 | 80 | 80 |
| I-134 | 80 | 80 |
| I-139 | 80 | 80 |
| I-141 | 80 | 90 |
| I-144 | 80 | 90 |
| I-145 | 80 | 90 |
| I-146 | 80 | 90 |
| I-148 | 80 | 80 |
| I-15 | 80 | 80 |
| I-155 | 80 | 80 |
| I-157 | 80 | 80 |
| I-16 | 80 | 80 |
| I-160 | 80 | 80 |
| I-162 | 80 | 80 |
| I-164 | 80 | 80 |
| I-165 | 80 | 90 |
| I-166 | 80 | 80 |
| I-167 | 80 | 80 |
| I-168 | 80 | 80 |
| I-169 | 80 | 80 |
| I-171 | 80 | 80 |
| I-172 | 80 | 80 |
| I-174 | 80 | 80 |
| I-175 | 80 | 80 |
| I-177 | 80 | 100 |
| I-179 | 80 | 90 |
| I-180 | 80 | 80 |
| I-181 | 80 | 90 |
| I-182 | 80 | 80 |
| I-184 | 80 | 90 |
| I-185 | 80 | 80 |
| I-187 | 80 | 90 |
| I-188 | 80 | 80 |
| I-189 | 80 | 90 |
| I-190 | 80 | 80 |
| I-191 | 80 | 80 |
| I-192 | 80 | 80 |
| I-193 | 80 | 80 |
| I-195 | 80 | 80 |
| I-196 | 80 | 80 |
| I-197 | 80 | 90 |
| I-198 | 80 | 80 |
| I-199 | 80 | 90 |
| I-204 | 80 | 90 |
| I-207 | 80 | 80 |
| I-208 | 80 | 80 |
| I-210 | 80 | 90 |
| I-211 | 80 | 80 |
| I-212 | 80 | 80 |
| I-218 | 80 | 80 |
| I-225 | 80 | 80 |
| I-227 | 80 | 90 |
| I-23 | 80 | 80 |
| I-230 | 80 | 80 |
| I-239 | 80 | 80 |
| I-247 | 80 | 80 |
| I-25 | 80 | 80 |
| I-251 | 80 | 80 |
| I-252 | 80 | 80 |
| I-264 | 80 | 80 |
| I-266 | 80 | 80 |
| I-267 | 80 | 90 |
| I-27 | 80 | 80 |
| I-273 | 80 | 80 |
| I-274 | 80 | 90 |
| I-275 | 80 | 90 |
| I-276 | 80 | 80 |
| I-278 | 80 | 80 |
| I-279 | 80 | 80 |
| I-285 | 80 | 80 |
| I-287 | 80 | 80 |
| I-288 | 80 | 80 |
| I-289 | 80 | 80 |
| I-29 | 80 | 80 |
| I-290 | 80 | 80 |
| I-294 | 80 | 90 |
| I-298 | 80 | 80 |
| I-299 | 80 | 80 |
| I-304 | 80 | 80 |
| I-307 | 80 | 90 |
| I-308 | 80 | 90 |
| I-309 | 80 | 80 |
| I-313 | 80 | 90 |
| I-314 | 80 | 90 |
| I-315 | 80 | 80 |
| I-316 | 80 | 90 |
| I-317 | 80 | 90 |
| I-318 | 80 | 90 |
| I-32 | 80 | 80 |
| I-322 | 80 | 80 |
| I-323 | 80 | 90 |
| I-324 | 80 | 90 |
| I-325 | 80 | 80 |
| I-326 | 80 | 90 |
| I-327 | 80 | 80 |
| I-328 | 80 | 90 |
| I-33 | 80 | 90 |
| I-330 | 80 | 80 |
| I-331 | 80 | 90 |
| I-335 | 80 | 90 |
| I-336 | 80 | 80 |
| I-337 | 80 | 80 |
| I-339 | 80 | 80 |
| I-34 | 80 | 90 |
| I-340 | 80 | 80 |
| I-341 | 80 | 90 |
| I-342 | 80 | 90 |
| I-343 | 80 | 80 |
| I-344 | 80 | 90 |
| I-348 | 80 | 80 |
| I-35 | 80 | 100 |
| I-352 | 80 | 80 |
| I-360 | 80 | 80 |
| I-366 | 80 | 80 |
| I-369 | 80 | 80 |
| I-37 | 80 | 80 |
| I-370 | 80 | 90 |
| I-374 | 80 | 80 |
| I-375 | 80 | 80 |
| I-38 | 80 | 80 |
| I-380 | 80 | 80 |
| I-382 | 80 | 80 |
| I-383 | 80 | 80 |
| I-388 | 80 | 80 |
| I-39 | 80 | 80 |
| I-40 | 80 | 80 |
| I-41 | 80 | 80 |
| I-42 | 80 | 90 |
| I-44 | 80 | 80 |
| I-45 | 80 | 80 |
| I-46 | 80 | 80 |
| I-48 | 80 | 90 |
| I-51 | 80 | 80 |
| I-52 | 80 | 90 |
| I-53 | 80 | 80 |
| I-56 | 80 | 80 |
| I-59 | 80 | 90 |
| I-64 | 80 | 90 |
| I-66 | 80 | 90 |
| I-73 | 80 | 80 |
| I-74 | 80 | 80 |
| I-75 | 80 | 80 |
| I-76 | 80 | 80 |
| I-77 | 80 | 90 |
| I-79 | 80 | 80 |
| I-81 | 80 | 90 |
| I-83 | 80 | 80 |
| I-84 | 80 | 80 |
| I-85 | 80 | 90 |
| I-87 | 80 | 80 |
| I-88 | 80 | 80 |
| I-90 | 80 | 90 |
| I-91 | 80 | 80 |
| I-93 | 80 | 80 |
| I-95 | 80 | 80 |
| I-96 | 80 | 80 |
| I-97 | 80 | 90 |
| I-98 | 80 | 80 |
| I-99 | 80 | 80 |
| II-38 | 80 | 80 |
| II-39 | 80 | 80 |
| II-40 | 80 | 80 |
| II-41 | 80 | 90 |
| II-42 | 80 | 80 |
| II-43 | 80 | 90 |
| II-44 | 80 | 80 |
| II-45 | 80 | 90 |
| III-22 | 80 | 80 |

TABLE B12b
Post-emergence action at 320 g/ha against VERPE in %

| Example number | Dosage [g/ha] | VERPE |
|---|---|---|
| I-01 | 320 | 90 |
| I-02 | 320 | 80 |
| I-03 | 320 | 90 |
| I-05 | 320 | 90 |
| I-07 | 320 | 90 |
| I-08 | 320 | 90 |
| I-10 | 320 | 80 |
| I-11 | 320 | 80 |
| I-13 | 320 | 80 |
| I-130 | 320 | 80 |
| I-133 | 320 | 80 |
| I-134 | 320 | 80 |
| I-137 | 320 | 80 |
| I-138 | 320 | 80 |
| I-139 | 320 | 90 |
| I-140 | 320 | 90 |
| I-141 | 320 | 90 |
| I-144 | 320 | 90 |
| I-145 | 320 | 90 |
| I-146 | 320 | 90 |
| I-148 | 320 | 80 |
| I-149 | 320 | 80 |
| I-15 | 320 | 80 |
| I-151 | 320 | 90 |
| I-152 | 320 | 80 |
| I-155 | 320 | 80 |
| I-156 | 320 | 80 |
| I-157 | 320 | 90 |
| I-16 | 320 | 90 |
| I-160 | 320 | 90 |
| I-161 | 320 | 80 |
| I-162 | 320 | 90 |
| I-164 | 320 | 80 |
| I-165 | 320 | 90 |
| I-166 | 320 | 80 |
| I-167 | 320 | 80 |
| I-168 | 320 | 90 |
| I-169 | 320 | 90 |
| I-17 | 320 | 80 |
| I-171 | 320 | 90 |
| I-172 | 320 | 90 |
| I-174 | 320 | 80 |
| I-175 | 320 | 80 |
| I-177 | 320 | 100 |
| I-179 | 320 | 90 |
| I-180 | 320 | 90 |
| I-181 | 320 | 90 |
| I-182 | 320 | 90 |
| I-183 | 320 | 90 |
| I-184 | 320 | 90 |
| I-185 | 320 | 80 |
| I-186 | 320 | 80 |
| I-187 | 320 | 90 |
| I-188 | 320 | 90 |
| I-189 | 320 | 90 |
| I-190 | 320 | 90 |
| I-191 | 320 | 90 |
| I-192 | 320 | 80 |
| I-193 | 320 | 80 |
| I-194 | 320 | 80 |
| I-195 | 320 | 80 |
| I-196 | 320 | 90 |
| I-197 | 320 | 90 |
| I-198 | 320 | 80 |
| I-199 | 320 | 90 |
| I-20 | 320 | 80 |
| I-202 | 320 | 80 |
| I-204 | 320 | 90 |
| I-206 | 320 | 80 |
| I-207 | 320 | 90 |
| I-208 | 320 | 80 |
| I-210 | 320 | 90 |
| I-211 | 320 | 90 |
| I-212 | 320 | 80 |
| I-218 | 320 | 80 |
| I-223 | 320 | 80 |
| I-225 | 320 | 90 |
| I-227 | 320 | 90 |
| I-230 | 320 | 80 |
| I-231 | 320 | 80 |
| I-235 | 320 | 80 |
| I-236 | 320 | 80 |
| I-239 | 320 | 80 |
| I-241 | 320 | 80 |
| I-243 | 320 | 80 |
| I-246 | 320 | 80 |
| I-247 | 320 | 80 |
| I-248 | 320 | 80 |
| I-249 | 320 | 80 |
| I-251 | 320 | 90 |
| I-252 | 320 | 80 |
| I-26 | 320 | 80 |
| I-260 | 320 | 80 |
| I-261 | 320 | 80 |
| I-264 | 320 | 90 |
| I-266 | 320 | 90 |
| I-267 | 320 | 90 |
| I-268 | 320 | 80 |
| I-27 | 320 | 90 |
| I-272 | 320 | 80 |
| I-273 | 320 | 90 |
| I-274 | 320 | 90 |
| I-275 | 320 | 90 |
| I-276 | 320 | 90 |
| I-277 | 320 | 80 |
| I-278 | 320 | 90 |
| I-279 | 320 | 90 |
| I-28 | 320 | 80 |
| I-285 | 320 | 80 |
| I-287 | 320 | 80 |
| I-288 | 320 | 80 |
| I-289 | 320 | 80 |
| I-290 | 320 | 80 |
| I-291 | 320 | 80 |
| I-294 | 320 | 90 |
| I-296 | 320 | 80 |
| I-297 | 320 | 80 |
| I-298 | 320 | 80 |
| I-299 | 320 | 80 |
| I-300 | 320 | 80 |
| I-302 | 320 | 80 |
| I-304 | 320 | 90 |
| I-305 | 320 | 90 |
| I-306 | 320 | 90 |
| I-307 | 320 | 90 |
| I-308 | 320 | 90 |
| I-309 | 320 | 80 |
| I-310 | 320 | 80 |
| I-311 | 320 | 90 |
| I-312 | 320 | 80 |
| I-313 | 320 | 90 |
| I-314 | 320 | 90 |
| I-315 | 320 | 90 |
| I-316 | 320 | 90 |
| I-317 | 320 | 90 |
| I-318 | 320 | 90 |
| I-319 | 320 | 80 |
| I-320 | 320 | 90 |
| I-321 | 320 | 90 |
| I-322 | 320 | 80 |
| I-323 | 320 | 90 |
| I-324 | 320 | 90 |
| I-325 | 320 | 90 |
| I-326 | 320 | 90 |
| I-327 | 320 | 90 |
| I-328 | 320 | 90 |
| I-330 | 320 | 80 |
| I-331 | 320 | 90 |
| I-332 | 320 | 80 |
| I-333 | 320 | 90 |
| I-334 | 320 | 80 |
| I-335 | 320 | 90 |
| I-336 | 320 | 90 |
| I-337 | 320 | 80 |
| I-338 | 320 | 90 |
| I-339 | 320 | 90 |
| I-340 | 320 | 90 |
| I-341 | 320 | 90 |
| I-342 | 320 | 90 |
| I-343 | 320 | 80 |
| I-344 | 320 | 90 |
| I-345 | 320 | 80 |
| I-348 | 320 | 80 |
| I-350 | 320 | 90 |
| I-351 | 320 | 80 |
| I-352 | 320 | 80 |
| I-354 | 320 | 90 |
| I-358 | 320 | 80 |
| I-360 | 320 | 80 |
| I-366 | 320 | 90 |
| I-369 | 320 | 80 |
| I-370 | 320 | 90 |
| I-373 | 320 | 90 |
| I-375 | 320 | 90 |
| I-376 | 320 | 80 |
| I-377 | 320 | 80 |
| I-378 | 320 | 80 |
| I-380 | 320 | 90 |
| I-381 | 320 | 80 |
| I-382 | 320 | 80 |
| I-383 | 320 | 90 |
| I-388 | 320 | 80 |
| I-41 | 320 | 80 |
| I-42 | 320 | 90 |
| I-48 | 320 | 90 |
| I-66 | 320 | 90 |
| I-80 | 320 | 80 |
| I-85 | 320 | 90 |
| I-86 | 320 | 80 |
| I-87 | 320 | 90 |
| I-88 | 320 | 80 |
| I-94 | 320 | 90 |
| I-95 | 320 | 80 |
| I-96 | 320 | 90 |
| II-37 | 320 | 90 |
| II-38 | 320 | 90 |
| II-39 | 320 | 90 |
| II-40 | 320 | 90 |
| II-41 | 320 | 90 |
| II-42 | 320 | 90 |
| II-43 | 320 | 90 |
| II-44 | 320 | 90 |
| II-45 | 320 | 90 |
| II-46 | 320 | 90 |
| III-01 | 320 | 80 |
| III-22 | 320 | 90 |
| III-23 | 320 | 90 |
| III-24 | 320 | 80 |
| III-29 | 320 | 80 |
| III-30 | 320 | 80 |

TABLE B13a
Post-emergence action at 80 g/ha against VIOTR in %

| Example number | Dosage [g/ha] | VIOTR |
|---|---|---|
| I-01 | 80 | 90 |
| I-03 | 80 | 90 |
| I-05 | 80 | 100 |
| I-07 | 80 | 90 |
| I-08 | 80 | 80 |
| I-101 | 80 | 80 |
| I-121 | 80 | 80 |
| I-130 | 80 | 90 |
| I-133 | 80 | 80 |
| I-136 | 80 | 80 |
| I-137 | 80 | 80 |
| I-138 | 80 | 80 |
| I-139 | 80 | 80 |
| I-140 | 80 | 90 |
| I-141 | 80 | 80 |
| I-144 | 80 | 90 |
| I-145 | 80 | 90 |
| I-146 | 80 | 90 |
| I-148 | 80 | 80 |
| I-149 | 80 | 80 |
| I-151 | 80 | 80 |
| I-155 | 80 | 80 |
| I-16 | 80 | 80 |
| I-165 | 80 | 90 |
| I-166 | 80 | 80 |
| I-167 | 80 | 80 |
| I-168 | 80 | 80 |
| I-17 | 80 | 80 |
| I-171 | 80 | 90 |
| I-172 | 80 | 80 |
| I-174 | 80 | 80 |
| I-175 | 80 | 80 |
| I-177 | 80 | 100 |
| I-179 | 80 | 90 |
| I-180 | 80 | 80 |
| I-181 | 80 | 90 |
| I-182 | 80 | 80 |
| I-183 | 80 | 90 |
| I-184 | 80 | 80 |
| I-185 | 80 | 80 |
| I-187 | 80 | 80 |
| I-188 | 80 | 80 |
| I-189 | 80 | 90 |
| I-19 | 80 | 90 |
| I-190 | 80 | 80 |
| I-193 | 80 | 80 |
| I-194 | 80 | 80 |
| I-195 | 80 | 80 |
| I-196 | 80 | 80 |
| I-197 | 80 | 90 |
| I-199 | 80 | 80 |
| I-20 | 80 | 90 |
| I-202 | 80 | 80 |
| I-204 | 80 | 80 |
| I-208 | 80 | 80 |
| I-211 | 80 | 80 |
| I-212 | 80 | 80 |
| I-216 | 80 | 80 |
| I-217 | 80 | 80 |
| I-218 | 80 | 80 |
| I-221 | 80 | 80 |
| I-225 | 80 | 80 |
| I-227 | 80 | 90 |
| I-229 | 80 | 80 |
| I-235 | 80 | 80 |
| I-239 | 80 | 80 |
| I-242 | 80 | 80 |
| I-244 | 80 | 80 |
| I-246 | 80 | 80 |
| I-247 | 80 | 80 |
| I-248 | 80 | 80 |
| I-249 | 80 | 80 |
| I-264 | 80 | 90 |
| I-266 | 80 | 80 |
| I-267 | 80 | 90 |
| I-268 | 80 | 80 |
| I-27 | 80 | 80 |
| I-273 | 80 | 90 |
| I-274 | 80 | 80 |
| I-275 | 80 | 90 |
| I-28 | 80 | 80 |
| I-285 | 80 | 80 |
| I-287 | 80 | 80 |
| I-289 | 80 | 80 |
| I-291 | 80 | 80 |
| I-293 | 80 | 80 |
| I-294 | 80 | 90 |
| I-296 | 80 | 80 |
| I-298 | 80 | 80 |
| I-299 | 80 | 80 |
| I-30 | 80 | 80 |
| I-300 | 80 | 80 |
| I-304 | 80 | 80 |
| I-307 | 80 | 80 |
| I-308 | 80 | 90 |
| I-310 | 80 | 80 |
| I-311 | 80 | 80 |
| I-314 | 80 | 90 |
| I-316 | 80 | 90 |
| I-317 | 80 | 90 |
| I-318 | 80 | 80 |
| I-319 | 80 | 80 |
| I-32 | 80 | 80 |
| I-321 | 80 | 80 |
| I-322 | 80 | 80 |
| I-323 | 80 | 90 |
| I-324 | 80 | 80 |
| I-325 | 80 | 90 |
| I-326 | 80 | 90 |
| I-327 | 80 | 90 |
| I-328 | 80 | 80 |
| I-331 | 80 | 80 |
| I-334 | 80 | 80 |
| I-335 | 80 | 80 |
| I-337 | 80 | 80 |
| I-339 | 80 | 90 |
| I-340 | 80 | 90 |
| I-341 | 80 | 80 |
| I-342 | 80 | 90 |
| I-343 | 80 | 80 |
| I-344 | 80 | 80 |
| I-347 | 80 | 80 |
| I-348 | 80 | 80 |
| I-35 | 80 | 90 |
| I-350 | 80 | 90 |
| I-354 | 80 | 80 |
| I-358 | 80 | 90 |
| I-36 | 80 | 90 |
| I-362 | 80 | 80 |
| I-366 | 80 | 80 |
| I-369 | 80 | 80 |
| I-375 | 80 | 80 |
| I-38 | 80 | 80 |
| I-380 | 80 | 90 |
| I-382 | 80 | 80 |
| I-383 | 80 | 80 |
| I-39 | 80 | 80 |
| I-41 | 80 | 80 |
| I-42 | 80 | 90 |
| I-48 | 80 | 80 |
| I-51 | 80 | 80 |
| I-56 | 80 | 80 |
| I-59 | 80 | 80 |
| I-64 | 80 | 80 |
| I-66 | 80 | 80 |
| I-69 | 80 | 80 |
| I-85 | 80 | 100 |
| I-86 | 80 | 80 |
| I-94 | 80 | 90 |
| I-96 | 80 | 90 |
| I-97 | 80 | 80 |
| II-36 | 80 | 80 |
| II-38 | 80 | 90 |
| II-39 | 80 | 80 |
| II-40 | 80 | 90 |
| II-41 | 80 | 80 |
| II-42 | 80 | 80 |
| II-43 | 80 | 90 |
| II-44 | 80 | 80 |
| II-45 | 80 | 90 |
| III-22 | 80 | 90 |
| III-24 | 80 | 80 |

TABLE B13b
Post-emergence action at 320 g/ha against VIOTR in %

| Example number | Dosage [g/ha] | VIOTR |
|---|---|---|
| I-01 | 320 | 100 |
| I-02 | 320 | 80 |
| I-03 | 320 | 90 |
| I-04 | 320 | 80 |
| I-05 | 320 | 100 |
| I-07 | 320 | 90 |
| I-08 | 320 | 80 |
| I-10 | 320 | 80 |
| I-11 | 320 | 80 |
| I-13 | 320 | 80 |
| I-130 | 320 | 90 |
| I-133 | 320 | 90 |
| I-136 | 320 | 80 |
| I-137 | 320 | 80 |
| I-138 | 320 | 80 |
| I-139 | 320 | 90 |
| I-140 | 320 | 90 |
| I-141 | 320 | 80 |
| I-144 | 320 | 90 |
| I-145 | 320 | 90 |
| I-146 | 320 | 90 |
| I-147 | 320 | 90 |
| I-148 | 320 | 80 |
| I-149 | 320 | 80 |
| I-15 | 320 | 80 |
| I-150 | 320 | 80 |
| I-151 | 320 | 90 |
| I-153 | 320 | 80 |
| I-154 | 320 | 80 |
| I-155 | 320 | 80 |
| I-159 | 320 | 80 |
| I-16 | 320 | 80 |
| I-161 | 320 | 80 |
| I-163 | 320 | 80 |
| I-164 | 320 | 80 |
| I-165 | 320 | 90 |
| I-166 | 320 | 80 |
| I-167 | 320 | 80 |
| I-168 | 320 | 90 |
| I-169 | 320 | 80 |
| I-17 | 320 | 80 |
| I-171 | 320 | 90 |
| I-172 | 320 | 90 |
| I-174 | 320 | 80 |
| I-175 | 320 | 80 |
| I-176 | 320 | 80 |
| I-177 | 320 | 100 |
| I-179 | 320 | 90 |
| I-18 | 320 | 80 |
| I-180 | 320 | 80 |
| I-181 | 320 | 90 |
| I-182 | 320 | 90 |
| I-183 | 320 | 90 |
| I-184 | 320 | 90 |
| I-185 | 320 | 80 |
| I-187 | 320 | 90 |
| I-188 | 320 | 80 |
| I-189 | 320 | 90 |
| I-190 | 320 | 90 |
| I-191 | 320 | 80 |
| I-192 | 320 | 80 |
| I-193 | 320 | 90 |
| I-194 | 320 | 80 |
| I-195 | 320 | 80 |
| I-196 | 320 | 80 |
| I-197 | 320 | 90 |
| I-198 | 320 | 80 |
| I-199 | 320 | 80 |
| I-20 | 320 | 90 |
| I-202 | 320 | 80 |
| I-204 | 320 | 80 |
| I-206 | 320 | 80 |
| I-207 | 320 | 80 |
| I-208 | 320 | 80 |
| I-211 | 320 | 90 |
| I-212 | 320 | 80 |
| I-216 | 320 | 80 |
| I-217 | 320 | 90 |
| I-218 | 320 | 80 |
| I-221 | 320 | 80 |
| I-223 | 320 | 80 |
| I-225 | 320 | 80 |
| I-227 | 320 | 90 |
| I-229 | 320 | 80 |
| I-230 | 320 | 80 |
| I-231 | 320 | 80 |
| I-235 | 320 | 80 |
| I-237 | 320 | 80 |
| I-238 | 320 | 80 |
| I-239 | 320 | 80 |
| I-240 | 320 | 80 |
| I-241 | 320 | 80 |
| I-242 | 320 | 80 |
| I-243 | 320 | 80 |
| I-244 | 320 | 80 |
| I-245 | 320 | 80 |
| I-246 | 320 | 80 |
| I-247 | 320 | 80 |
| I-248 | 320 | 80 |
| I-249 | 320 | 80 |
| I-251 | 320 | 90 |
| I-252 | 320 | 80 |
| I-26 | 320 | 80 |
| I-261 | 320 | 80 |
| I-264 | 320 | 90 |
| I-266 | 320 | 90 |
| I-267 | 320 | 90 |
| I-268 | 320 | 80 |
| I-27 | 320 | 80 |
| I-273 | 320 | 90 |
| I-274 | 320 | 90 |
| I-275 | 320 | 90 |
| I-28 | 320 | 80 |
| I--284 | 320 | 80 |
| I-285 | 320 | 80 |
| I-286 | 320 | 80 |
| I-287 | 320 | 90 |
| I-288 | 320 | 80 |
| I-289 | 320 | 80 |
| I-290 | 320 | 80 |
| I-291 | 320 | 80 |
| I-293 | 320 | 80 |
| I-294 | 320 | 90 |
| I-296 | 320 | 80 |
| I-298 | 320 | 90 |
| I-299 | 320 | 90 |
| I-300 | 320 | 90 |
| I-302 | 320 | 80 |
| I-304 | 320 | 90 |
| I-306 | 320 | 80 |
| I-307 | 320 | 90 |
| I-308 | 320 | 90 |
| I-31 | 320 | 80 |
| I-310 | 320 | 90 |
| I-311 | 320 | 80 |
| I-313 | 320 | 80 |
| I-314 | 320 | 90 |
| I-315 | 320 | 80 |
| I-316 | 320 | 90 |
| I-317 | 320 | 90 |
| I-318 | 320 | 90 |
| I-319 | 320 | 90 |
| I-320 | 320 | 80 |
| I-321 | 320 | 80 |
| I-322 | 320 | 80 |
| I-323 | 320 | 90 |
| I-324 | 320 | 80 |
| I-325 | 320 | 90 |
| I-326 | 320 | 90 |
| I-327 | 320 | 90 |
| I-328 | 320 | 90 |
| I-330 | 320 | 80 |
| I-331 | 320 | 80 |
| I-332 | 320 | 80 |
| I-333 | 320 | 80 |
| I-334 | 320 | 80 |
| I-335 | 320 | 90 |
| I-336 | 320 | 80 |
| I-337 | 320 | 80 |
| I-338 | 320 | 80 |
| I-339 | 320 | 90 |
| I-340 | 320 | 90 |
| I-341 | 320 | 80 |
| I-342 | 320 | 90 |
| I-343 | 320 | 80 |
| I-344 | 320 | 80 |
| I-347 | 320 | 80 |
| I-348 | 320 | 80 |
| I-349 | 320 | 80 |
| I-350 | 320 | 90 |
| I-354 | 320 | 80 |
| I-358 | 320 | 90 |
| I-362 | 320 | 80 |
| I-366 | 320 | 90 |
| I-369 | 320 | 80 |
| I-370 | 320 | 80 |
| I-375 | 320 | 90 |
| I-376 | 320 | 80 |
| I-380 | 320 | 90 |
| I-382 | 320 | 90 |
| I-383 | 320 | 90 |
| I-388 | 320 | 80 |
| I-41 | 320 | 80 |
| I-42 | 320 | 90 |
| I-48 | 320 | 90 |
| I-66 | 320 | 90 |
| I-69 | 320 | 80 |
| I-82 | 320 | 80 |
| I-85 | 320 | 100 |
| I-86 | 320 | 90 |
| I-94 | 320 | 90 |
| I-95 | 320 | 80 |
| I-96 | 320 | 90 |
| II-37 | 320 | 80 |
| II-38 | 320 | 90 |
| II-39 | 320 | 90 |
| II-40 | 320 | 90 |
| II-41 | 320 | 90 |
| II-42 | 320 | 90 |
| II-43 | 320 | 90 |
| II-44 | 320 | 90 |
| II-45 | 320 | 90 |
| II-46 | 320 | 90 |
| III-01 | 320 | 80 |
| III-08 | 320 | 80 |
| III-22 | 320 | 90 |
| III-23 | 320 | 90 |
| III-24 | 320 | 90 |
| III-27 | 320 | 80 |
| III-30 | 320 | 80 |

Tables B14 to B18 below show the crop plant compatibilities of selected compounds of the general formula (I) according to Table 1 at an application rate corresponding to 320 g/ha or less, which were observed in trials by the experimental procedure mentioned above. The observed effects on selected crop plants are reported here in comparison to the untreated controls (values in %). The appendices “a”, “b” and “c” give differentiation by dosage used with otherwise the same crop plants tested.

TABLE B14a
Post-emergence action at 20 g/ha against ZEAMX in %

| Example number | Dosage [g/ha] | ZEAMX |
|---|---|---|
| I-01 | 20 | 0 |
| I-11 | 20 | 10 |
| I-16 | 20 | 10 |
| I-13 | 20 | 0 |
| I-17 | 20 | 10 |
| I-19 | 20 | 0 |
| I-126 | 20 | 10 |
| I-39 | 20 | 0 |
| I-55 | 20 | 0 |
| I-54 | 20 | 0 |
| I-36 | 20 | 20 |
| I-56 | 20 | 0 |
| I-122 | 20 | 0 |
| I-100 | 20 | 10 |
| I-102 | 20 | 0 |
| I-101 | 20 | 10 |
| I-33 | 20 | 10 |
| I-32 | 20 | 20 |
| I-107 | 20 | 20 |
| I-99 | 20 | 0 |
| I-98 | 20 | 10 |
| I-91 | 20 | 20 |
| I-93 | 20 | 20 |
| I-90 | 20 | 0 |
| I-29 | 20 | 10 |
| I-46 | 20 | 0 |
| I-30 | 20 | 20 |
| I-45 | 20 | 0 |
| I-44 | 20 | 0 |
| I-23 | 20 | 0 |
| I-24 | 20 | 10 |
| I-47 | 20 | 10 |
| I-116 | 20 | 20 |
| I-53 | 20 | 10 |
| I-374 | 20 | 10 |
| I-114 | 20 | 0 |
| I-119 | 20 | 10 |
| I-108 | 20 | 0 |
| I-109 | 20 | 10 |
| I-110 | 20 | 10 |
| I-112 | 20 | 0 |
| I-25 | 20 | 10 |
| I-52 | 20 | 0 |
| I-35 | 20 | 10 |
| I-34 | 20 | 10 |
| I-51 | 20 | 20 |
| I-50 | 20 | 0 |
| I-49 | 20 | 0 |
| III-21 | 20 | 0 |
| III-20 | 20 | 0 |
| III-19 | 20 | 0 |
| I-81 | 20 | 0 |
| III-17 | 20 | 20 |
| I-75 | 20 | 10 |
| I-74 | 20 | 10 |
| I-77 | 20 | 0 |
| I-76 | 20 | 20 |
| III-18 | 20 | 0 |
| I-40 | 20 | 0 |
| I-71 | 20 | 0 |
| I-70 | 20 | 0 |
| I-43 | 20 | 10 |
| III-29 | 20 | 10 |
| I-252 | 20 | 10 |
| I-314 | 20 | 0 |
| I-344 | 20 | 0 |
| I-328 | 20 | 0 |
| I-318 | 20 | 0 |
| I-326 | 20 | 0 |
| I-299 | 20 | 0 |
| II-44 | 20 | 0 |
| II-38 | 20 | 0 |
| II-40 | 20 | 0 |
| II-41 | 20 | 0 |
| II-43 | 20 | 0 |
| II-39 | 20 | 0 |
| II-37 | 20 | 0 |
| II-42 | 20 | 20 |
| I-286 | 20 | 10 |
| I-290 | 20 | 0 |
| I-169 | 20 | 10 |
| I-136 | 20 | 10 |
| I-134 | 20 | 10 |
| I-168 | 20 | 20 |
| I-166 | 20 | 10 |
| I-285 | 20 | 10 |
| II-46 | 20 | 0 |
| I-139 | 20 | 0 |
| I-137 | 20 | 0 |
| I-138 | 20 | 20 |
| I-130 | 20 | 0 |
| I-133 | 20 | 0 |
| I-181 | 20 | 20 |
| I-182 | 20 | 10 |
| I-304 | 20 | 20 |
| I-383 | 20 | 10 |
| I-294 | 20 | 20 |
| I-251 | 20 | 20 |
| I-366 | 20 | 10 |
| I-382 | 20 | 0 |
| I-380 | 20 | 0 |
| I-287 | 20 | 10 |
| I-264 | 20 | 10 |
| I-274 | 20 | 10 |
| I-289 | 20 | 0 |
| I-275 | 20 | 0 |
| I-267 | 20 | 0 |
| I-387 | 20 | 0 |
| I-249 | 20 | 0 |
| I-261 | 20 | 0 |
| I-268 | 20 | 0 |
| I-248 | 20 | 0 |
| I--284 | 20 | 0 |

TABLE B14b
Post-emergence action at 80 g/ha against ZEAMX in %

| Example number | Dosage [g/ha] | ZEAMX |
|---|---|---|
| I-01 | 80 | 0 |
| I-03 | 80 | 0 |
| I-04 | 80 | 0 |
| I-05 | 80 | 0 |
| I-07 | 80 | 0 |
| I-10 | 80 | 0 |
| I-101 | 80 | 10 |
| I-102 | 80 | 20 |
| I-108 | 80 | 10 |
| I-109 | 80 | 10 |
| I-110 | 80 | 10 |
| I-112 | 80 | 10 |
| I-114 | 80 | 0 |
| I-130 | 80 | 0 |
| I-133 | 80 | 0 |
| I-136 | 80 | 10 |
| I-137 | 80 | 0 |
| I-138 | 80 | 20 |
| I-139 | 80 | 0 |
| I-147 | 80 | 0 |
| I-150 | 80 | 0 |
| I-153 | 80 | 10 |
| I-157 | 80 | 20 |
| I-16 | 80 | 20 |
| I-161 | 80 | 10 |
| I-169 | 80 | 10 |
| I-179 | 80 | 20 |
| I-182 | 80 | 20 |
| I-186 | 80 | 20 |
| I-200 | 80 | 0 |
| I-201 | 80 | 0 |
| I-205 | 80 | 0 |
| I-225 | 80 | 20 |
| I-23 | 80 | 10 |
| I-230 | 80 | 0 |
| I-235 | 80 | 20 |
| I-236 | 80 | 0 |
| I-237 | 80 | 10 |
| I-238 | 80 | 10 |
| I-239 | 80 | 0 |
| I-24 | 80 | 20 |
| I-240 | 80 | 10 |
| I-241 | 80 | 0 |
| I-242 | 80 | 10 |
| I-243 | 80 | 0 |
| I-244 | 80 | 20 |
| I-246 | 80 | 10 |
| I-248 | 80 | 0 |
| I-249 | 80 | 20 |
| I-25 | 80 | 20 |
| I-251 | 80 | 20 |
| I-26 | 80 | 0 |
| I-261 | 80 | 0 |
| I-264 | 80 | 20 |
| I-267 | 80 | 20 |
| I-268 | 80 | 0 |
| I-272 | 80 | 10 |
| I-274 | 80 | 20 |
| I-275 | 80 | 20 |
| I-277 | 80 | 10 |
| I-278 | 80 | 20 |
| I-28 | 80 | 0 |
| I--284 | 80 | 0 |
| I-286 | 80 | 20 |
| I-287 | 80 | 20 |
| I-289 | 80 | 10 |
| I-29 | 80 | 20 |
| I-290 | 80 | 10 |
| I-299 | 80 | 10 |
| I-30 | 80 | 20 |
| I-302 | 80 | 0 |
| I-305 | 80 | 0 |
| I-31 | 80 | 0 |
| I-310 | 80 | 0 |
| I-311 | 80 | 10 |
| I-312 | 80 | 10 |
| I-314 | 80 | 10 |
| I-315 | 80 | 20 |
| I-316 | 80 | 0 |
| I-318 | 80 | 0 |
| I-319 | 80 | 10 |
| I-320 | 80 | 0 |
| I-321 | 80 | 0 |
| I-323 | 80 | 0 |
| I-324 | 80 | 0 |
| I-325 | 80 | 0 |
| I-326 | 80 | 0 |
| I-327 | 80 | 0 |
| I-328 | 80 | 0 |
| I-329 | 80 | 0 |
| I-330 | 80 | 10 |
| I-332 | 80 | 10 |
| I-334 | 80 | 10 |
| I-335 | 80 | 0 |
| I-337 | 80 | 10 |
| I-338 | 80 | 0 |
| I-34 | 80 | 10 |
| I-341 | 80 | 0 |
| I-344 | 80 | 0 |
| I-345 | 80 | 0 |
| I-346 | 80 | 0 |
| I-347 | 80 | 20 |
| I-348 | 80 | 20 |
| I-35 | 80 | 10 |
| I-351 | 80 | 10 |
| I-354 | 80 | 0 |
| I-357 | 80 | 0 |
| I-358 | 80 | 0 |
| I-362 | 80 | 0 |
| I-363 | 80 | 0 |
| I-369 | 80 | 10 |
| I-371 | 80 | 20 |
| I-373 | 80 | 0 |
| I-376 | 80 | 10 |
| I-377 | 80 | 0 |
| I-378 | 80 | 10 |
| I-380 | 80 | 20 |
| I-381 | 80 | 0 |
| I-382 | 80 | 20 |
| I-383 | 80 | 20 |
| I-386 | 80 | 0 |
| I-387 | 80 | 0 |
| I-40 | 80 | 20 |
| I-41 | 80 | 20 |
| I-43 | 80 | 10 |
| I-44 | 80 | 10 |
| I-45 | 80 | 10 |
| I-46 | 80 | 10 |
| I-47 | 80 | 10 |
| I-48 | 80 | 20 |
| I-49 | 80 | 10 |
| I-50 | 80 | 0 |
| I-52 | 80 | 10 |
| I-55 | 80 | 0 |
| I-56 | 80 | 0 |
| I-58 | 80 | 20 |
| I-63 | 80 | 0 |
| I-70 | 80 | 10 |
| I-71 | 80 | 10 |
| I-75 | 80 | 20 |
| I-80 | 80 | 20 |
| I-81 | 80 | 0 |
| I-90 | 80 | 10 |
| I-94 | 80 | 20 |
| II-37 | 80 | 0 |
| II-38 | 80 | 0 |
| II-39 | 80 | 0 |
| II-40 | 80 | 0 |
| II-41 | 80 | 0 |
| II-43 | 80 | 20 |
| II-44 | 80 | 0 |
| II-46 | 80 | 0 |
| III-01 | 80 | 0 |
| III-02 | 80 | 10 |
| III-07 | 80 | 0 |
| III-09 | 80 | 10 |
| III-17 | 80 | 20 |
| III-18 | 80 | 0 |
| III-21 | 80 | 0 |
| III-23 | 80 | 0 |
| III-25 | 80 | 0 |
| III-26 | 80 | 20 |
| III-27 | 80 | 10 |
| III-28 | 80 | 0 |
| III-30 | 80 | 20 |
| I-10 | 320 | 20 |
| I-130 | 320 | 20 |
| I-133 | 320 | 20 |
| I-136 | 320 | 20 |
| I-137 | 320 | 20 |
| I-139 | 320 | 10 |
| I-147 | 320 | 20 |
| I-150 | 320 | 0 |
| I-161 | 320 | 10 |
| I-200 | 320 | 0 |
| I-201 | 320 | 20 |
| I-205 | 320 | 0 |
| I-239 | 320 | 20 |
| I-240 | 320 | 20 |
| I--284 | 320 | 0 |
| I-297 | 320 | 0 |
| I-302 | 320 | 10 |
| I-31 | 320 | 0 |
| I-320 | 320 | 20 |
| I-329 | 320 | 0 |
| I-334 | 320 | 20 |
| I-335 | 320 | 20 |
| I-345 | 320 | 10 |
| I-346 | 320 | 0 |
| I-357 | 320 | 10 |
| I-363 | 320 | 0 |
| I-371 | 320 | 20 |
| I-386 | 320 | 0 |
| I-387 | 320 | 0 |
| I-62 | 320 | 20 |
| I-63 | 320 | 10 |
| II-37 | 320 | 0 |
| II-38 | 320 | 0 |
| II-39 | 320 | 20 |
| II-40 | 320 | 10 |
| II-46 | 320 | 20 |
| III-01 | 320 | 0 |
| III-02 | 320 | 20 |
| III-07 | 320 | 0 |
| III-09 | 320 | 10 |
| III-25 | 320 | 0 |
| III-27 | 320 | 10 |
| III-28 | 320 | 0 |

TABLE B15a
Post-emergence action at 20 g/ha against TRZAS in %

| Example number | Dosage [g/ha] | TRZAS |
|---|---|---|
| I-103 | 20 | 0 |
| I-108 | 20 | 0 |
| I-109 | 20 | 0 |
| I-110 | 20 | 0 |
| I-112 | 20 | 0 |
| I-114 | 20 | 0 |
| I-119 | 20 | 20 |
| I-125 | 20 | 10 |
| I-126 | 20 | 20 |
| I-130 | 20 | 20 |
| I-134 | 20 | 20 |
| I-136 | 20 | 10 |
| I-137 | 20 | 20 |
| I-138 | 20 | 10 |
| I-139 | 20 | 0 |
| I-140 | 20 | 10 |
| I-145 | 20 | 20 |
| I-16 | 20 | 20 |
| I-165 | 20 | 20 |
| I-168 | 20 | 10 |
| I-169 | 20 | 0 |
| I-17 | 20 | 10 |
| I-179 | 20 | 0 |
| I-181 | 20 | 20 |
| I-182 | 20 | 10 |
| I-248 | 20 | 0 |
| I-249 | 20 | 0 |
| I-251 | 20 | 10 |
| I-261 | 20 | 0 |
| I-266 | 20 | 20 |
| I-268 | 20 | 0 |
| I-273 | 20 | 0 |
| I--284 | 20 | 0 |
| I-286 | 20 | 20 |
| I-287 | 20 | 20 |
| I-289 | 20 | 20 |
| I-294 | 20 | 10 |
| I-380 | 20 | 20 |
| I-383 | 20 | 20 |
| I-387 | 20 | 0 |
| I-44 | 20 | 20 |
| I-47 | 20 | 10 |
| I-49 | 20 | 0 |
| I-50 | 20 | 10 |
| I-51 | 20 | 20 |
| I-55 | 20 | 0 |
| I-70 | 20 | 0 |
| I-71 | 20 | 10 |
| I-72 | 20 | 10 |
| I-74 | 20 | 0 |
| I-75 | 20 | 20 |
| I-78 | 20 | 0 |
| I-99 | 20 | 20 |
| II-37 | 20 | 0 |
| II-38 | 20 | 0 |
| II-39 | 20 | 0 |
| II-40 | 20 | 0 |
| II-41 | 20 | 0 |
| II-42 | 20 | 0 |
| II-43 | 20 | 10 |
| II-44 | 20 | 20 |
| II-45 | 20 | 0 |
| II-46 | 20 | 0 |
| III-17 | 20 | 20 |
| III-18 | 20 | 0 |
| III-19 | 20 | 20 |
| III-20 | 20 | 0 |
| III-21 | 20 | 0 |
| III-29 | 20 | 20 |

TABLE 15b
Post-emergence action at 80 g/ha against TRZAS in %

| Example number | Dosage [g/ha] | TRZAS |
|---|---|---|
| I-04 | 80 | 20 |
| I-108 | 80 | 20 |
| I-109 | 80 | 10 |
| I-111 | 80 | 0 |
| I-112 | 80 | 0 |
| I-114 | 80 | 0 |
| I-137 | 80 | 20 |
| I-147 | 80 | 0 |
| I-150 | 80 | 0 |
| I-160 | 80 | 20 |
| I-161 | 80 | 0 |
| I-162 | 80 | 10 |
| I-186 | 80 | 10 |
| I-187 | 80 | 20 |
| I-200 | 80 | 0 |
| I-201 | 80 | 0 |
| I-202 | 80 | 20 |
| I-205 | 80 | 10 |
| I-225 | 80 | 20 |
| I-230 | 80 | 10 |
| I-243 | 80 | 20 |
| I-248 | 80 | 0 |
| I-249 | 80 | 0 |
| I-261 | 80 | 0 |
| I-268 | 80 | 0 |
| I-276 | 80 | 0 |
| I-277 | 80 | 0 |
| I-278 | 80 | 0 |
| I-279 | 80 | 10 |
| I--284 | 80 | 0 |
| I-297 | 80 | 10 |
| I-31 | 80 | 10 |
| I-312 | 80 | 20 |
| I-329 | 80 | 0 |
| I-345 | 80 | 10 |
| I-346 | 80 | 10 |
| I-351 | 80 | 0 |
| I-352 | 80 | 10 |
| I-354 | 80 | 0 |
| I-357 | 80 | 10 |
| I-363 | 80 | 0 |
| I-371 | 80 | 20 |
| I-376 | 80 | 0 |
| I-377 | 80 | 0 |
| I-387 | 80 | 0 |
| I-49 | 80 | 10 |
| I-50 | 80 | 10 |
| I-55 | 80 | 0 |
| I-62 | 80 | 0 |
| I-63 | 80 | 0 |
| I-69 | 80 | 0 |
| I-70 | 80 | 10 |
| I-78 | 80 | 20 |
| II-37 | 80 | 0 |
| II-38 | 80 | 10 |
| II-39 | 80 | 20 |
| II-40 | 80 | 20 |
| II-41 | 80 | 0 |
| II-42 | 80 | 20 |
| II-44 | 80 | 20 |
| II-46 | 80 | 0 |
| III-01 | 80 | 0 |
| III-02 | 80 | 20 |
| III-07 | 80 | 0 |
| III-08 | 80 | 0 |
| III-09 | 80 | 0 |
| III-18 | 80 | 0 |
| III-20 | 80 | 0 |
| III-21 | 80 | 0 |
| III-23 | 80 | 0 |
| III-24 | 80 | 0 |
| III-25 | 80 | 0 |
| III-26 | 80 | 20 |

TABLE B15c
Post-emergence action at 320 g/ha against TRZAS in %

| Example number | Dosage [g/ha] | TRZAS |
|---|---|---|
| I-147 | 320 | 20 |
| I-150 | 320 | 10 |
| I-161 | 320 | 10 |
| I-200 | 320 | 0 |
| I-201 | 320 | 10 |
| I-205 | 320 | 10 |
| I-249 | 320 | 0 |
| I-276 | 320 | 20 |
| I-277 | 320 | 20 |
| I--284 | 320 | 0 |
| I-351 | 320 | 10 |
| I-357 | 320 | 10 |
| I-376 | 320 | 20 |
| I-387 | 320 | 0 |
| I-62 | 320 | 0 |
| I-63 | 320 | 0 |
| II-37 | 320 | 0 |
| III-09 | 320 | 0 |
| III-24 | 320 | 0 |

TABLE B16a
Post-emergence action at 20 g/ha against ORYSA in %

| Example number | Dosage [g/ha] | ORYSA |
|---|---|---|
| I-01 | 20 | 0 |
| I-100 | 20 | 0 |
| I-101 | 20 | 0 |
| I-102 | 20 | 0 |
| I-103 | 20 | 0 |
| I-107 | 20 | 0 |
| I-108 | 20 | 0 |
| I-109 | 20 | 0 |
| I-11 | 20 | 0 |
| I-110 | 20 | 0 |
| I-112 | 20 | 0 |
| I-114 | 20 | 0 |
| I-116 | 20 | 10 |
| I-118 | 20 | 0 |
| I-119 | 20 | 0 |
| I-120 | 20 | 10 |
| I-121 | 20 | 0 |
| I-122 | 20 | 0 |
| I-125 | 20 | 0 |
| I-126 | 20 | 0 |
| I-13 | 20 | 0 |
| I-130 | 20 | 0 |
| I-133 | 20 | 0 |
| I-134 | 20 | 0 |
| I-136 | 20 | 10 |
| I-137 | 20 | 0 |
| I-138 | 20 | 0 |
| I-139 | 20 | 0 |
| I-140 | 20 | 0 |
| I-141 | 20 | 10 |
| I-144 | 20 | 0 |
| I-145 | 20 | 0 |
| I-146 | 20 | 0 |
| I-15 | 20 | 0 |
| I-16 | 20 | 0 |
| I-165 | 20 | 0 |
| I-166 | 20 | 0 |
| I-168 | 20 | 10 |
| I-169 | 20 | 0 |
| I-17 | 20 | 0 |
| I-172 | 20 | 20 |
| I-176 | 20 | 0 |
| I-177 | 20 | 0 |
| I-179 | 20 | 0 |
| I-18 | 20 | 0 |
| I-181 | 20 | 0 |
| I-182 | 20 | 10 |
| I-19 | 20 | 0 |
| I-197 | 20 | 0 |
| I-227 | 20 | 0 |
| I-23 | 20 | 0 |
| I-24 | 20 | 0 |
| I-25 | 20 | 0 |
| I-251 | 20 | 10 |
| I-252 | 20 | 10 |
| I-260 | 20 | 10 |
| I-264 | 20 | 0 |
| I-266 | 20 | 20 |
| I-274 | 20 | 0 |
| I-285 | 20 | 10 |
| I-286 | 20 | 10 |
| I-287 | 20 | 0 |
| I-288 | 20 | 0 |
| I-289 | 20 | 0 |
| I-29 | 20 | 10 |
| I-290 | 20 | 0 |
| I-294 | 20 | 10 |
| I-299 | 20 | 10 |
| I-314 | 20 | 0 |
| I-318 | 20 | 0 |
| I-32 | 20 | 0 |
| I-326 | 20 | 0 |
| I-328 | 20 | 0 |
| I-33 | 20 | 0 |
| I-34 | 20 | 10 |
| I-344 | 20 | 0 |
| I-35 | 20 | 0 |
| I-36 | 20 | 0 |
| I-366 | 20 | 0 |
| I-374 | 20 | 10 |
| I-380 | 20 | 0 |
| I-382 | 20 | 0 |
| I-39 | 20 | 0 |
| I-43 | 20 | 10 |
| I-44 | 20 | 20 |
| I-45 | 20 | 10 |
| I-46 | 20 | 10 |
| I-47 | 20 | 20 |
| I-49 | 20 | 0 |
| I-50 | 20 | 10 |
| I-51 | 20 | 20 |
| I-52 | 20 | 0 |
| I-53 | 20 | 10 |
| I-54 | 20 | 0 |
| I-55 | 20 | 0 |
| I-56 | 20 | 0 |
| I-59 | 20 | 0 |
| I-64 | 20 | 10 |
| I-70 | 20 | 0 |
| I-71 | 20 | 10 |
| I-72 | 20 | 10 |
| I-73 | 20 | 0 |
| I-74 | 20 | 0 |
| I-75 | 20 | 0 |
| I-76 | 20 | 0 |
| I-77 | 20 | 0 |
| I-78 | 20 | 0 |
| I-79 | 20 | 0 |
| I-81 | 20 | 0 |
| I-83 | 20 | 0 |
| I-84 | 20 | 0 |
| I-90 | 20 | 0 |
| I-91 | 20 | 0 |
| I-93 | 20 | 10 |
| I-97 | 20 | 0 |
| I-98 | 20 | 10 |
| I-99 | 20 | 0 |
| II-36 | 20 | 0 |
| II-37 | 20 | 0 |
| II-38 | 20 | 0 |
| II-39 | 20 | 0 |
| II-40 | 20 | 0 |
| II-41 | 20 | 0 |
| II-42 | 20 | 0 |
| II-43 | 20 | 0 |
| II-44 | 20 | 0 |
| II-45 | 20 | 0 |
| II-46 | 20 | 0 |
| III-17 | 20 | 0 |
| III-18 | 20 | 0 |
| III-19 | 20 | 0 |
| III-20 | 20 | 0 |
| III-21 | 20 | 0 |
| III-29 | 20 | 10 |

TABLE B16b
Post-emergence action at 80 g/ha against ORYSA in %

| Example number | Dosage [g/ha] | ORYSA |
|---|---|---|
| I-01 | 80 | 0 |
| I-03 | 80 | 0 |
| I-04 | 80 | 0 |
| I-05 | 80 | 0 |
| I-07 | 80 | 0 |
| I-101 | 80 | 10 |
| I-102 | 80 | 0 |
| I-103 | 80 | 0 |
| I-107 | 80 | 10 |
| I-108 | 80 | 10 |
| I-109 | 80 | 0 |
| I-11 | 80 | 0 |
| I-110 | 80 | 10 |
| I-111 | 80 | 0 |
| I-112 | 80 | 0 |
| I-114 | 80 | 0 |
| I-116 | 80 | 10 |
| I-118 | 80 | 0 |
| I-119 | 80 | 10 |
| I-120 | 80 | 20 |
| I-121 | 80 | 0 |
| I-122 | 80 | 0 |
| I-125 | 80 | 0 |
| I-126 | 80 | 0 |
| I-13 | 80 | 10 |
| I-133 | 80 | 10 |
| I-134 | 80 | 10 |
| I-136 | 80 | 20 |
| I-137 | 80 | 10 |
| I-138 | 80 | 0 |
| I-139 | 80 | 0 |
| I-140 | 80 | 0 |
| I-147 | 80 | 0 |
| I-15 | 80 | 10 |
| I-150 | 80 | 0 |
| I-151 | 80 | 0 |
| I-152 | 80 | 0 |
| I-153 | 80 | 0 |
| I-155 | 80 | 10 |
| I-156 | 80 | 0 |
| I-157 | 80 | 10 |
| I-159 | 80 | 10 |
| I-16 | 80 | 10 |
| I-160 | 80 | 0 |
| I-161 | 80 | 0 |
| I-162 | 80 | 0 |
| I-166 | 80 | 20 |
| I-169 | 80 | 10 |
| I-17 | 80 | 10 |
| I-175 | 80 | 20 |
| I-176 | 80 | 10 |
| I-179 | 80 | 20 |
| I-18 | 80 | 10 |
| I-185 | 80 | 20 |
| I-186 | 80 | 10 |
| I-187 | 80 | 10 |
| I-188 | 80 | 20 |
| I-19 | 80 | 0 |
| I-190 | 80 | 20 |
| I-192 | 80 | 0 |
| I-193 | 80 | 10 |
| I-194 | 80 | 20 |
| I-197 | 80 | 10 |
| I-200 | 80 | 0 |
| I-201 | 80 | 0 |
| I-202 | 80 | 20 |
| I-204 | 80 | 0 |
| I-205 | 80 | 0 |
| I-207 | 80 | 10 |
| I-208 | 80 | 0 |
| I-210 | 80 | 10 |
| I-212 | 80 | 10 |
| I-216 | 80 | 10 |
| I-217 | 80 | 0 |
| I-221 | 80 | 20 |
| I-223 | 80 | 10 |
| I-225 | 80 | 10 |
| I-229 | 80 | 0 |
| I-23 | 80 | 10 |
| I-230 | 80 | 0 |
| I-231 | 80 | 10 |
| I-235 | 80 | 0 |
| I-236 | 80 | 0 |
| I-237 | 80 | 0 |
| I-238 | 80 | 0 |
| I-239 | 80 | 0 |
| I-24 | 80 | 10 |
| I-240 | 80 | 0 |
| I-241 | 80 | 0 |
| I-242 | 80 | 0 |
| I-243 | 80 | 0 |
| I-245 | 80 | 0 |
| I-246 | 80 | 0 |
| I-247 | 80 | 10 |
| I-25 | 80 | 10 |
| I-260 | 80 | 10 |
| I-264 | 80 | 10 |
| I-27 | 80 | 0 |
| I-272 | 80 | 10 |
| I-276 | 80 | 10 |
| I-277 | 80 | 10 |
| I-278 | 80 | 0 |
| I-279 | 80 | 10 |
| I-28 | 80 | 0 |
| I-285 | 80 | 10 |
| I-286 | 80 | 20 |
| I-287 | 80 | 20 |
| I-288 | 80 | 10 |
| I-289 | 80 | 10 |
| I-29 | 80 | 20 |
| I-290 | 80 | 10 |
| I-291 | 80 | 0 |
| I-293 | 80 | 0 |
| I-296 | 80 | 0 |
| I-298 | 80 | 20 |
| I-299 | 80 | 20 |
| I-302 | 80 | 20 |
| I-306 | 80 | 0 |
| I-31 | 80 | 0 |
| I-310 | 80 | 10 |
| I-311 | 80 | 20 |
| I-312 | 80 | 10 |
| I-313 | 80 | 10 |
| I-314 | 80 | 20 |
| I-315 | 80 | 20 |
| I-319 | 80 | 0 |
| I-32 | 80 | 0 |
| I-320 | 80 | 0 |
| I-321 | 80 | 0 |
| I-322 | 80 | 10 |
| I-325 | 80 | 20 |
| I-327 | 80 | 0 |
| I-329 | 80 | 0 |
| I-33 | 80 | 10 |
| I-330 | 80 | 10 |
| I-332 | 80 | 20 |
| I-333 | 80 | 20 |
| I-334 | 80 | 20 |
| I-335 | 80 | 0 |
| I-336 | 80 | 20 |
| I-337 | 80 | 20 |
| I-338 | 80 | 0 |
| I-339 | 80 | 0 |
| I-34 | 80 | 20 |
| I-340 | 80 | 0 |
| I-341 | 80 | 0 |
| I-343 | 80 | 10 |
| I-344 | 80 | 0 |
| I-345 | 80 | 20 |
| I-346 | 80 | 0 |
| I-347 | 80 | 0 |
| I-348 | 80 | 0 |
| I-349 | 80 | 0 |
| I-35 | 80 | 0 |
| I-350 | 80 | 0 |
| I-351 | 80 | 0 |
| I-352 | 80 | 20 |
| I-357 | 80 | 0 |
| I-358 | 80 | 0 |
| I-359 | 80 | 0 |
| I-36 | 80 | 0 |
| I-360 | 80 | 20 |
| I-362 | 80 | 10 |
| I-363 | 80 | 0 |
| I-366 | 80 | 20 |
| I-369 | 80 | 20 |
| I-370 | 80 | 0 |
| I-371 | 80 | 0 |
| I-373 | 80 | 0 |
| I-376 | 80 | 0 |
| I-377 | 80 | 0 |
| I-378 | 80 | 10 |
| I-380 | 80 | 10 |
| I-381 | 80 | 20 |
| I-382 | 80 | 10 |
| I-386 | 80 | 0 |
| I-41 | 80 | 0 |
| I-47 | 80 | 20 |
| I-49 | 80 | 10 |
| I-50 | 80 | 10 |
| I-52 | 80 | 10 |
| I-54 | 80 | 0 |
| I-55 | 80 | 0 |
| I-56 | 80 | 0 |
| I-58 | 80 | 0 |
| I-59 | 80 | 0 |
| I-60 | 80 | 0 |
| I-62 | 80 | 0 |
| I-63 | 80 | 0 |
| I-65 | 80 | 0 |
| I-66 | 80 | 10 |
| I-69 | 80 | 0 |
| I-70 | 80 | 0 |
| I-71 | 80 | 20 |
| I-72 | 80 | 20 |
| I-73 | 80 | 10 |
| I-74 | 80 | 10 |
| I-75 | 80 | 0 |
| I-76 | 80 | 0 |
| I-78 | 80 | 20 |
| I-79 | 80 | 0 |
| I-80 | 80 | 20 |
| I-81 | 80 | 0 |
| I-82 | 80 | 20 |
| I-83 | 80 | 20 |
| I-84 | 80 | 10 |
| I-87 | 80 | 0 |
| I-88 | 80 | 0 |
| I-90 | 80 | 0 |
| I-91 | 80 | 20 |
| I-93 | 80 | 20 |
| I-94 | 80 | 20 |
| I-95 | 80 | 0 |
| I-97 | 80 | 10 |
| I-99 | 80 | 0 |
| II-36 | 80 | 0 |
| II-37 | 80 | 0 |
| II-38 | 80 | 0 |
| II-39 | 80 | 0 |
| II-40 | 80 | 0 |
| II-41 | 80 | 0 |
| II-42 | 80 | 0 |
| II-43 | 80 | 0 |
| II-44 | 80 | 0 |
| II-45 | 80 | 0 |
| II-46 | 80 | 0 |
| III-01 | 80 | 0 |
| III-02 | 80 | 0 |
| III-07 | 80 | 0 |
| III-08 | 80 | 0 |
| III-09 | 80 | 0 |
| III-17 | 80 | 0 |
| III-18 | 80 | 0 |
| III-19 | 80 | 0 |
| III-20 | 80 | 0 |
| III-21 | 80 | 20 |
| III-22 | 80 | 0 |
| III-23 | 80 | 0 |
| III-24 | 80 | 0 |
| III-25 | 80 | 0 |
| III-26 | 80 | 10 |
| III-27 | 80 | 0 |
| III-28 | 80 | 0 |
| III-29 | 80 | 20 |
| III-30 | 80 | 10 |

TABLE B16c
Post-emergence action at 320 g/ha against ORYSA in %

| Example number | Dosage [g/ha] | ORYSA |
|---|---|---|
| I-04 | 320 | 0 |
| I-11 | 320 | 10 |
| I-111 | 320 | 0 |
| I-137 | 320 | 20 |
| I-138 | 320 | 20 |
| I-139 | 320 | 10 |
| I-140 | 320 | 10 |
| I-147 | 320 | 0 |
| I-150 | 320 | 0 |
| I-161 | 320 | 0 |
| I-18 | 320 | 20 |
| I-186 | 320 | 20 |
| I-187 | 320 | 20 |
| I-200 | 320 | 0 |
| I-201 | 320 | 0 |
| I-205 | 320 | 10 |
| I-223 | 320 | 20 |
| I-235 | 320 | 0 |
| I-236 | 320 | 0 |
| I-237 | 320 | 20 |
| I-238 | 320 | 10 |
| I-240 | 320 | 0 |
| I-241 | 320 | 0 |
| I-242 | 320 | 0 |
| I-243 | 320 | 10 |
| I-244 | 320 | 0 |
| I-246 | 320 | 10 |
| I-278 | 320 | 10 |
| I-286 | 320 | 20 |
| I-31 | 320 | 10 |
| I-310 | 320 | 20 |
| I-329 | 320 | 0 |
| I-340 | 320 | 0 |
| I-346 | 320 | 0 |
| I-347 | 320 | 0 |
| I-349 | 320 | 10 |
| I-350 | 320 | 10 |
| I-357 | 320 | 10 |
| I-358 | 320 | 0 |
| I-359 | 320 | 0 |
| I-363 | 320 | 0 |
| I-371 | 320 | 0 |
| I-376 | 320 | 20 |
| I-381 | 320 | 20 |
| I-386 | 320 | 0 |
| I-60 | 320 | 0 |
| I-62 | 320 | 0 |
| I-63 | 320 | 10 |
| I-65 | 320 | 0 |
| I-66 | 320 | 20 |
| I-69 | 320 | 10 |
| I-80 | 320 | 20 |
| II-37 | 320 | 0 |
| II-38 | 320 | 0 |
| II-39 | 320 | 0 |
| II-40 | 320 | 0 |
| II-41 | 320 | 0 |
| II-42 | 320 | 0 |
| II-43 | 320 | 0 |
| II-46 | 320 | 0 |
| III-01 | 320 | 0 |
| III-02 | 320 | 20 |
| III-07 | 320 | 0 |
| III-08 | 320 | 0 |
| III-09 | 320 | 0 |
| III-23 | 320 | 20 |
| III-25 | 320 | 0 |
| III-26 | 320 | 20 |
| III-27 | 320 | 0 |
| III-28 | 320 | 0 |
| III-30 | 320 | 20 |

TABLE B17a
Post-emergence action at 20 g/ha against GLXMA in %

| Example number | Dosage [g/ha] | GLXMA |
|---|---|---|
| I-108 | 20 | 0 |
| I-112 | 20 | 0 |
| I-114 | 20 | 0 |
| I-125 | 20 | 20 |
| I-126 | 20 | 20 |
| I-261 | 20 | 0 |
| I--284 | 20 | 0 |
| I-49 | 20 | 10 |
| I-50 | 20 | 0 |
| I-55 | 20 | 0 |
| II-37 | 20 | 0 |
| III-20 | 20 | 0 |
| III-21 | 20 | 0 |

TABLE B17b
Post-emergence action at 80 g/ha against GLXMA in %

| Example number | Dosage [g/ha] | GLXMA |
|---|---|---|
| I-108 | 80 | 10 |
| I-112 | 80 | 10 |
| I-114 | 80 | 0 |
| I-147 | 80 | 0 |
| I-150 | 80 | 0 |
| I-161 | 80 | 0 |
| I-200 | 80 | 0 |
| I-201 | 80 | 20 |
| I--284 | 80 | 0 |
| I-349 | 80 | 0 |
| I-50 | 80 | 10 |
| I-55 | 80 | 0 |
| I-82 | 80 | 0 |
| III-20 | 80 | 0 |
| III-21 | 80 | 0 |

TABLE B17c
Post-emergence action at 320 g/ha against GLXMA in %

| Example number | Dosage [g/ha] | GLXMA |
|---|---|---|
| I-147 | 320 | 10 |
| I-150 | 320 | 0 |
| I-200 | 320 | 0 |
| I--284 | 320 | 0 |

TABLE B18a
Post-emergence action at 20 g/ha against BRSNW in %

| Example number | Dosage [g/ha] | BRSNW |
|---|---|---|
| I-108 | 20 | 10 |
| I-109 | 20 | 0 |
| I-110 | 20 | 10 |
| I-112 | 20 | 0 |
| I--284 | 20 | 0 |
| I-289 | 20 | 20 |
| I-47 | 20 | 10 |
| I-50 | 20 | 10 |
| I-55 | 20 | 0 |
| I-70 | 20 | 20 |
| II-37 | 20 | 0 |
| III-20 | 20 | 0 |
| III-21 | 20 | 0 |

TABLE B18b
Post-emergence action at 80 g/ha against BRSNW in %

| Example number | Dosage [g/ha] | BRSNW |
|---|---|---|
| I-109 | 80 | 10 |
| I-110 | 80 | 20 |
| I-112 | 80 | 10 |
| I-200 | 80 | 10 |
| I--284 | 80 | 0 |
| I-349 | 80 | 20 |
| I-50 | 80 | 20 |
| I-55 | 80 | 0 |
| I-62 | 80 | 20 |
| III-21 | 80 | 0 |

TABLE B18c
Post-emergence action at 320 g/ha against BRSNW in %

| Example number | Dosage [g/ha] | BRSNW |
|---|---|---|
| I-200 | 320 | 20 |
| I--284 | 320 | 0 |

As the results from Tables B1 to B18 show, inventive compounds of the general formula (I) in the case of post-emergence treatment have good herbicidal efficacy against harmful plants, for example Abutilon theophrasti (ABUTH), Alopecurus myosuroides (ALOMY), Amaranthus retroflexus (AMARE), Avena fatua (AVEFA), Digitaria sanguinalis (DIGSA), Echinochloa crus-galli (ECHCG), Lolium rigidum (LOLRI), Matricaria inodora (MATIN), Pharbitis purpurea (PHBPU), Polygonum convolvulus (POLCO), Setaria viridis (SETVI), Verconica persica (VERPE) and Viola tricolor (VIOTR) at an application rate of 320 g of active substance or less per hectare, and good crop plant compatibility in the case of organisms such as Oryza sativa (ORYSA), Zea mays (ZEAMX), Brassica napus (BRSNW), Glycine max (GLXMA) and Triticum aestivum (TRZAS) at an application rate of 320 g or less per hectare.

The compounds of the invention are therefore suitable for control of unwanted plant growth by the post-emergence method.

1. Compounds of general formula (I) and agrochemically compatible salts, N-oxides, hydrates, and hydrates of the salts and N-oxides thereof, 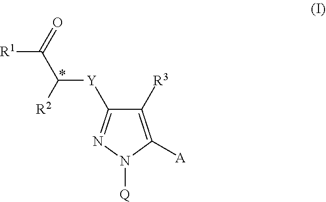
where
A is selected from the group consisting of A1-A15: 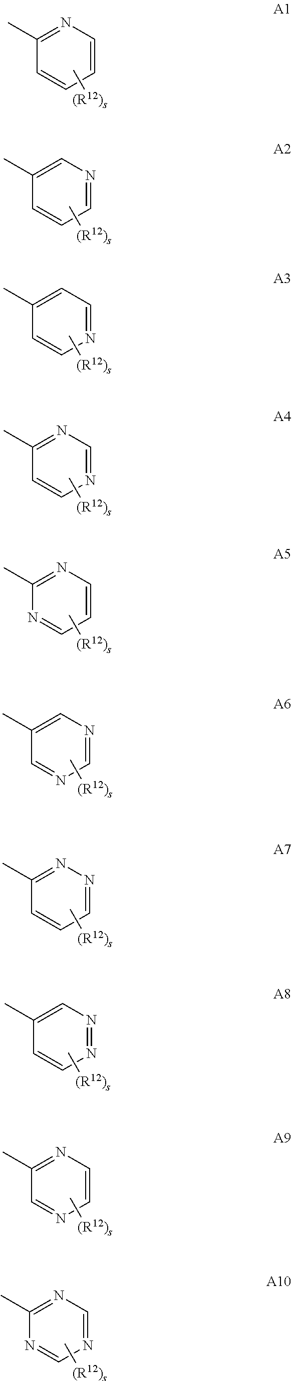 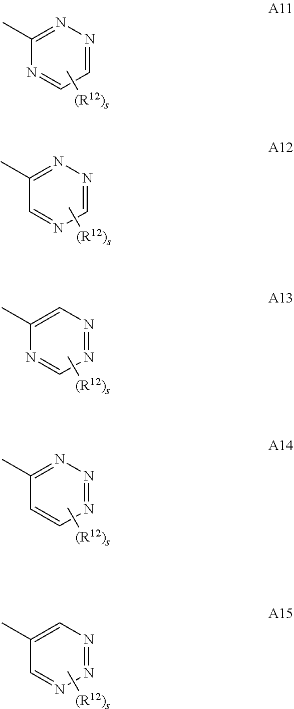
Q is selected from the group consisting of Q1-Q3: 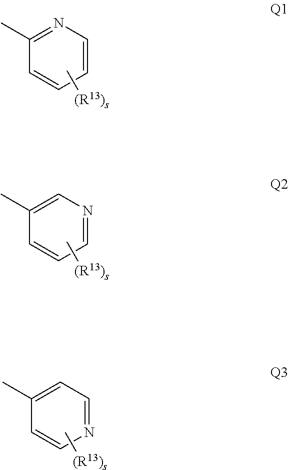
R¹ is —OR^{1a}, —NR⁹R¹⁰ or —O(CH₂)ₓO(CH₂)_{y}O(CH₂)_{z} H;
R^{1a} is hydrogen or
is (C₁-C₆)-alkyl, (C₃-C₆)-cycloalkyl, each of which is unsubstituted or substituted by one or more substituents selected from the group consisting of halogen, (C₁-C₆)-alkyl, (C₁-C₆)-haloalkyl, (C₃-C₆)-cycloalkyl, (C₁-C₆)-alkoxy, (C₁-C₂)-haloalkoxy, (C₁-C₆)-alkoxycarbonyl, cyano and nitro or is (C₂-C₄)-alkenyl, CH₂C(COOMe)=CH₂, (C₂-C₄)-alkynyl or is (C₁-C₆)-alkyl-S—(C₁-C₆)-alkyl-, (C₁-C₆)-alkyl-SO—(C₁-C₆)-alkyl-, (C₁-C₆)-alkyl-SO₂—(C₁-C₆)-alkyl- or is —N═(C₃-C₆)-cycloalkyl, —N═C(CH₃)₂ or
is heterocyclyl, heteroaryl, aryl or
is heterocyclyl-(C₁-C₄)-alkyl-, heteroaryl-(C₁-C₄)-alkyl-, aryl-(C₁-C₄)-alkyl-, each of which is unsubstituted or substituted by one or more substituents selected from the group consisting of halogen, (C₁-C₆)-alkyl, (C₁-C₆)-haloalkyl;
R⁹ is hydrogen, (C₁-C₁₂)-alkyl;
R¹⁰ is hydrogen, aryl, heteroaryl, heterocyclyl, (C₁-C₁₂)-alkyl, (C₃-C₈)-cycloalkyl, (C₃-C₈)-cycloalkyl-(C₁-C₇)-alkyl-, (C₂-C₁₂)-alkenyl, (C₅-C₇)-cycloalkenyl, (C₂-C₁₂)-alkynyl, S(O)ₙR⁵, cyano, nitro, OR⁵, SO₂NR⁶R⁷, CO₂R⁸, COR⁸, NR⁶R⁸, NR⁶COR⁸, NR⁶CO₂R⁸, NR⁶SO₂R⁸, where the abovementioned alkyl, cycloalkyl, alkenyl, cycloalkenyl and alkynyl radicals are unsubstituted or each independently substituted by “m” radicals selected from the group consisting of hydrogen, optionally mono- or polysubstituted aryl, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or
R⁹ and R¹⁰ together with the nitrogen atom to which they are bonded form a saturated or partly or fully unsaturated five-, six- or seven-membered ring which is optionally mono- to hexasubstituted by the following radicals from the group consisting of halogen, (C₁-C₆)-alkyl, (C₁-C₆)-haloalkyl, OR⁵, S(O)ₙRS, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl, aryl;
R⁶ is hydrogen, (C₁-C₆)-alkyl, (C₃-C₆)-cycloalkyl, (C₁-C₆)-haloalkyl, aryl;
R⁷ is hydrogen, (C₁-C₆)-alkyl, (C₃-C₆)-cycloalkyl, (C₃-C₄)-alkenyl, (C₃-C₄)-alkynyl, CH₂—C(O)O(C₁-C₂)-alkyl;
R⁸ is hydrogen, (C₁-C₆)-alkyl, (C₃-C₆)-cycloalkyl, (C₃-C₄)-alkenyl, (C₁-C₆)-alkyl-C(O)O(C₁-C₂)-alkyl or (C₃-C₄)-alkynyl;
R² is hydrogen, cyano, (C₁-C₆)-alkyl, (C₁-C₆)-alkoxy-(C₁-C₆)-alkyl-, (C₁-C₆)-haloalkyl, (C₁-C₆)-alkoxy, (C₂-C₆)-alkenyl, (C₂-C₆)-haloalkenyl, (C₂-C₆)-alkynyl, (C₂-C₆)-haloalkynyl, (C₃-C₆)-cycloalkyl;
R³ is halogen, cyano, isocyano, NO₂, (C₃-C₆)-cycloalkyl, (C₁-C₆)-alkyl, (C₃-C₆)-halocycloalkyl, (C₁-C₆)-haloalkyl, (C₁-C₆)-alkylcarbonyl-, (C₁-C₆)-haloalkylcarbonyl-, (C₁-C₆)-alkyloxycarbonyl-, (C₂-C₃)-alkenyl, (C₂-C₃)-haloalkenyl, (C₂-C₃)-alkynyl, (C₂-C₃)-haloalkynyl, (C₁-C₆)-alkyl-S(O)ₙ and (C₁-C₆)-haloalkyl-S(O)ₙ, CHO, NH₂;
R¹² is halogen, cyano, isocyano, NO₂, (C₁-C₆)-alkyl, (C₁-C₆)-haloalkyl, (C₁-C₆)-alkylcarbonyl, (C₁-C₆)-haloalkylcarbonyl, (C₁-C₆)-alkyloxycarbonyl, (C₁-C₆)-alkoxy, (C₁-C₆)-haloalkoxy, (C₁-C₆)-alkyl-, S(O)ₙ, (C₂-C₃)-alkenyl, (C₂-C₃)-haloalkenyl, (C₂-C₃)-alkynyl, (C₂-C₃)-haloalkynyl, NH₂;
R¹³ is halogen, cyano, isocyano, NO₂, (C₁-C₆)-alkyl, (C₁-C₆)-haloalkyl, (C₁-C₆)-alkylcarbonyl, (C₁-C₆)-haloalkylcarbonyl, (C₁-C₆)-alkyloxycarbonyl, (C₁-C₆)-alkoxy, (C₁-C₆)-haloalkoxy, (C₁-C₆)-alkyl-S(O)ₙ, (C₂-C₃)-alkenyl, (C₂-C₃)-haloalkenyl; (C₂-C₃)-alkynyl, (C₂-C₃)-haloalkynyl, NH₂;
Y is oxygen, S(O)ₙ;
m is 0, 1 or 2;
n is 0, 1 or 2;
o is 0, 1 or 2;
p is 0 or 1;
q is 0 or 1;
r is 3, 4, 5 or 6;
s is 0, 1, 2, 3 or 4;
x is 2, 3;
y is 2, 3;
z is 1, 2.

2. The compounds of the formula (I) according to claim 1 and their agrochemically compatible salts, N-oxides, hydrates, and hydrates of the salts and N-oxides, where
A is selected from the group consisting of A1-A4, A6, A8, A9, A12, A15 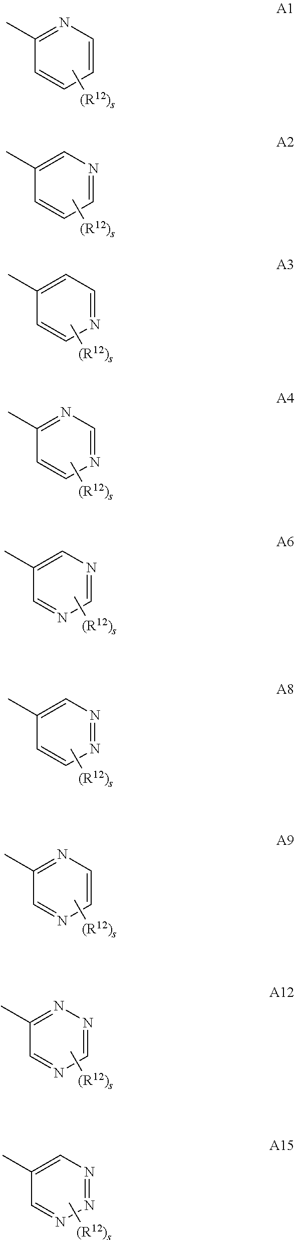
Q is selected from the group consisting of Q1-Q3: 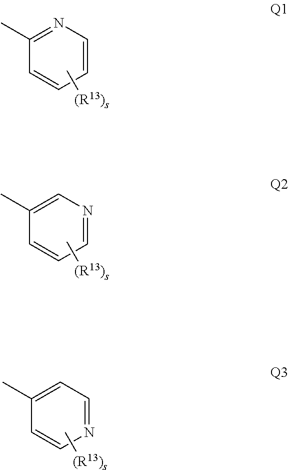
R¹ is —OR^{1a}, —NR⁹R¹⁰ or —O(CH₂)ₓO(CH₂)_{y}O(CH₂)_{z}H;
R^{1a} is hydrogen or
is (C₁-C₃)-alkyl, (C₃-C₆)-cycloalkyl, each of which is unsubstituted or substituted by one or more substituents selected from the group consisting of halogen, (C₁-C₄)-alkyl, (C₁-C₄)-haloalkyl, (C₃-C₆)-cycloalkyl, (C₁-C₄)-alkoxy, (C₁-C₄)-alkoxycarbonyl, cyano,
or is (C₂-C₄)-alkenyl, (C₂-C₄)-alkynyl, or
is (C₂-C₃)-alkyl-SMe-, (C₂-C₃)-alkyl-SOMe-, (C₂-C₃)-alkyl-SO₂Me-, or
is —N═C(CH₃)₂ or
is heterocyclyl, or
is heterocyclyl-(C₁-C₂)-alkyl-, heteroaryl-(C₁-C₂)-alkyl-, aryl-(C₁-C₂)-alkyl-, each of which is unsubstituted or substituted by one or more substituents selected from the group consisting of halogen, (C₁-C₂)-alkyl, (C₁-C₂)-haloalkyl;
R⁹ is hydrogen, (C₁-C₄)-alkyl;
R¹⁰ is (C₁-C₄)-alkyl, (C₂-C₄)-alkenyl, (C₂-C₄)-alkynyl, S(O)ₙR⁵, SO₂NR⁶R⁷, where the abovementioned alkyl, alkenyl and alkynyl radicals are unsubstituted or each independently substituted by “m” radicals selected from the group consisting of halogen, cyano, S(O)ₙRS, SO₂NR⁶R⁷, CO₂R⁸, CONR⁶R⁸,
or
R⁹ and R¹⁰ together with the nitrogen atom to which they are bonded form a saturated or partly or fully unsaturated five-, six- or seven-membered ring which is optionally mono- or disubstituted by the following radicals from the group consisting of (C₁-C₄)-alkyl, (C₁-C₄)-haloalkyl, CO₂R⁸ and CONR⁶R⁸;
R⁵ is (C₁-C₄)-alkyl, (C₃-C₆)-cycloalkyl, (C₁-C₄)-haloalkyl, phenyl;
R⁶ is hydrogen, (C₁-C₄)-alkyl, (C₃-C₆)-cycloalkyl, (C₁-C₄)-haloalkyl, phenyl;
R⁷ is hydrogen, (C₁-C₄)-alkyl, (C₃-C₆)-cycloalkyl, (C₃-C₄)-alkenyl, (C₃-C₄)-alkynyl;
R⁸ is hydrogen, (C₁-C₄)-alkyl, (C₃-C₆)-cycloalkyl, (C₃-C₄)-alkenyl, (C₁-C₄)-alkyl-C(O)O(C₁-C₂)-alkyl or (C₃-C₄)-alkynyl;
R² is hydrogen, (C₁-C₃)-alkyl, (C₁-C₃)-alkoxy, (C₂-C₃)-alkenyl, (C₂-C₃)-alkynyl, (C₃-C₆)-cycloalkyl;
R³ is halogen, cyano, isocyano, NO₂, (C₃-C₆)-cycloalkyl, (C₁-C₄)-alkyl, (C₃-C₆)-halocycloalkyl, (C₁-C₄)-haloalkyl, (C₁-C₄)-alkylcarbonyl-, (C₁-C₄)-haloalkylcarbonyl-, (C₁-C₄)-alkyloxycarbonyl-, (C₂-C₃)-alkenyl, (C₂-C₃)-haloalkenyl, (C₂-C₃)-alkynyl, (C₂-C₃)-haloalkynyl, CHO;
R¹² is halogen, cyano, NO₂, (C₁-C₄)-alkyl, (C₁-C₄)-haloalkyl, (C₁-C₄)-alkylcarbonyl, (C₁-C₄)-haloalkylcarbonyl, (C₁-C₄)-alkyloxycarbonyl, (C₁-C₄)-alkoxy, (C₁-C₄)-haloalkoxy, (C₂-C₃)-alkenyl, (C₂-C₃)-haloalkenyl, (C₂-C₃)-alkynyl, (C₂-C₃)-haloalkynyl, NH₂;
R¹³ is halogen, cyano, NO₂, (C₁-C₄)-alkyl, (C₁-C₄)-haloalkyl, (C₁-C₄)-alkylcarbonyl, (C₁-C₄)-haloalkylcarbonyl, (C₁-C₄)-alkyloxycarbonyl, (C₁-C₄)-alkoxy, (C₁-C₄)-haloalkoxy, (C₂-C₃)-alkenyl, (C₂-C₃)-haloalkenyl; (C₂-C₃)-alkynyl, (C₂-C₃)-haloalkynyl, NH₂;
Y is oxygen, S(O)ₙ;
m is 0 or 1;
n is 0, 1 or 2;
s is 0, 1, 2 or 3;
x is 2;
y is 2;
z is 1.

3. The compounds of the formula (I) according to claim 1 and their agrochemically compatible salts, N-oxides, hydrates, and hydrates of the salts and N-oxides, where
A is selected from the group consisting of A2, A3, A6 and A8 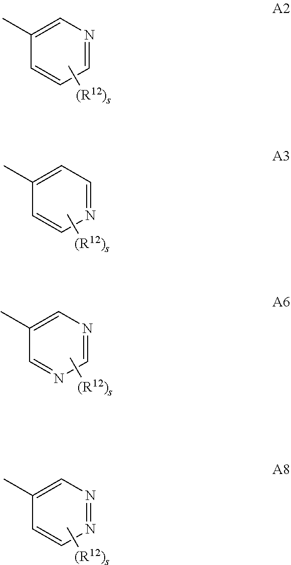
Q is selected from the group consisting of Q1-Q3: 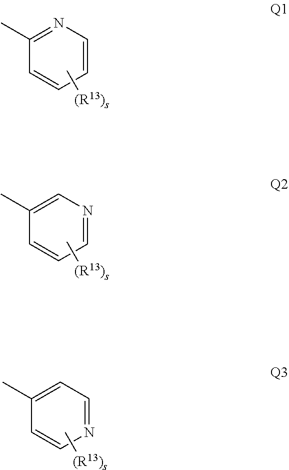
R¹ is —OR^{1a}, —NR⁹R¹⁰ or —O(CH₂)₂O(CH₂)₂OCH₃;
R^{1a} is hydrogen or
is (C₁-C₃)-alkyl which is unsubstituted or substituted by a substituent selected from the group consisting of —C(O)OMe, cyclopropyl, cyclopentyl, methoxy, cyano, trifluoromethyl or
is (C₃-C₆)-cycloalkyl or
is —N═C(CH₃)₂ or
is prop-2-ynyl or
is oxetan-3-yl-(C₁-C₂)-alkyl-, tetrahydrofuran-2-yl-(C₁-C₂)-alkyl-, tetrahydrofuran-3-yl-(C₁-C₂)-alkyl-, pyridin-2-yl-(C₁-C₂)-alkyl-, pyridin-3-yl-(C₁-C₂)-alkyl-, pyridin-4-yl-(C₁-C₂)-alkyl-, phenyl-(C₁-C₂)-alkyl-;
R⁹ is hydrogen;
R¹⁰ is (C₁-C₄)-alkyl where the alkyl radical is unsubstituted or monosubstituted by CO₂R⁸, S(O)₂R⁵ or SO₂NR⁶R⁷;
or
R⁹ and R¹⁰ together with the nitrogen atom to which they are bonded form a saturated five- or seven-membered ring monosubstituted by CO₂R⁸;
R⁵ is methyl, ethyl or CH₂CF₃;
R⁶ is hydrogen or methyl;
R⁷ is methyl or ethyl;
R⁸ is hydrogen, methyl or ethyl;
R² is hydrogen, methyl or ethyl;
R³ is halogen, cyano, NO₂, (C₃-C₅)-cycloalkyl, (C₃-C₅)-halocycloalkyl, (C₁-C₂)-alkyl, (C₁-C₂)-haloalkyl;
R¹² is fluorine or chlorine;
R¹³ is fluorine, chlorine, methyl, ethyl, methoxy, trifluoromethyl;
Y is oxygen;
s is 0, 1, 2.

4. The compounds of the formula (I) according to claim 1 and their agrochemically compatible salts, N-oxides, hydrates, and hydrates of the salts and N-oxides, where
A is selected from the group consisting of A2, A3 and A6 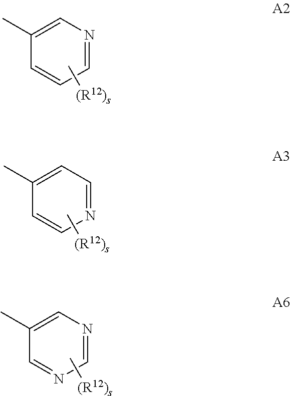
Q is selected from the group consisting of Q1-Q3: 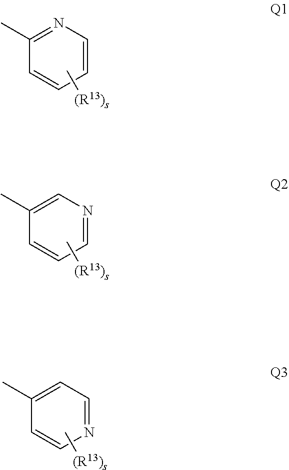
R¹ is —OR^{1a} or —NR⁹R¹⁰,
R^{1a} is hydrogen or
methyl, ethyl, each of which is unsubstituted or substituted by a substituent selected from the group consisting of —C(O)OMe, cyclopropyl, cyclopentyl, methoxy, cyano, trifluoromethyl or
is —N═C(CH₃)₂ or
is prop-2-ynyl or
—CH₂-tetrahydrofuran-2-yl, —CH₂-tetrahydrofuran-3-yl, —CH₂-oxetan-3-yl, —CH₂-pyridyl-2-yl, —CH₂-pyridin-3-yl, —CH₂-pyridin-4-yl, —CH₂-phenyl,
R⁹ is hydrogen;
R¹⁰ methyl or ethyl, where the radicals are monosubstituted by CO₂R⁸,
or
R⁹ and R¹⁰ together with the nitrogen atom to which they are bonded form a cyclopentyl or cyclohexyl radical monosubstituted by CO₂R⁸;
R⁸ is methyl or ethyl;
R² is hydrogen or methyl;
R³ is halogen, cyano, NO₂, cyclopropyl, cyclobutyl, 2,2-difluorocyclopropyl, trifluoromethyl;
R¹² is fluorine;
R¹³ is fluorine, chlorine, methyl, trifluoromethyl;
Y is oxygen;
s is 0, 1, 2.

5. The compounds of the formula (I) according to claim 1 and their agrochemically compatible salts, N-oxides, hydrates, and hydrates of the salts and N-oxides, where
A is selected from the group consisting of A2, A3 and A6 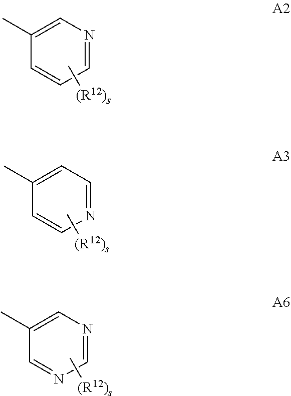
Q is selected from the group consisting of Q1-Q3: 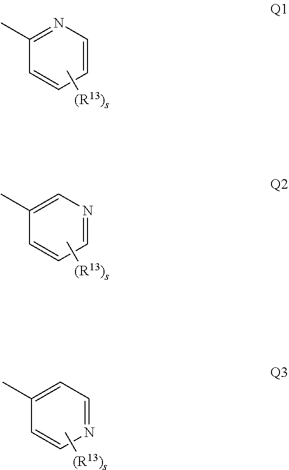
R¹ is —OR^{1a};
R^{1a} is hydrogen, methyl, ethyl;
R² is hydrogen or methyl;
R³ is fluorine, chlorine, bromine, cyano, NO₂, cyclopropyl, trifluoromethyl;
R¹² is fluorine;
R¹³ is fluorine, chlorine, trifluoromethyl;
Y is oxygen;
s is 0, 1, 2.

6. Compounds of formula (Z) with Q=Q1, R¹=—OR^{1a} and Y═O and their agrochemically compatible salts, N-oxides, hydrates, and hydrates of the salts and N-oxides, where 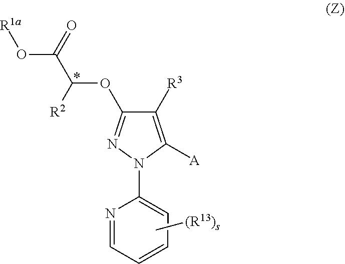
substituents are as set forth in claim 1.

7. Compounds of formula (Y) with Q=Q1, Y═O and A=A2 and their agrochemically compatible salts, N-oxides, hydrates, and hydrates of the salts and N-oxides, where 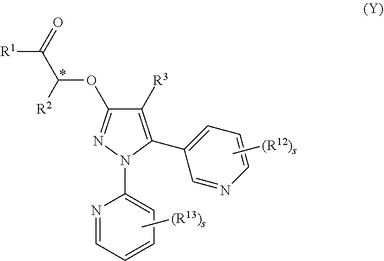
substituents are as set forth in claim 1.

8. A process for preparing compounds of formula (Ic) or an agrochemically acceptable salt thereof according to claim 1 by converting compounds of general formulae (III) and (IV) 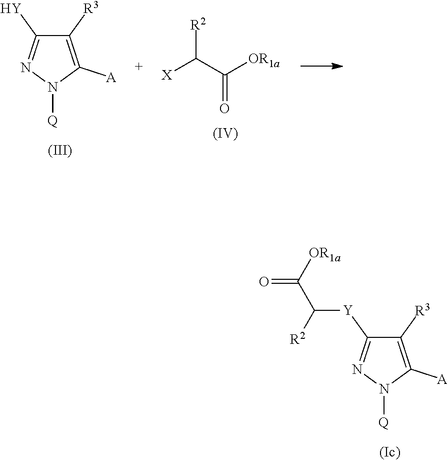
in which R², R^{1a}, R³, A, Q and Y are as set forth in claim 1 and X is chlorine, bromine or iodine, in the presence of a sulfurizing reagent.

9. A process for preparing compounds of formula (Ia) or an agrochemically acceptable salt thereof according to claim 1 by converting, via an ester cleavage reaction, a compound of general formula (Ic) 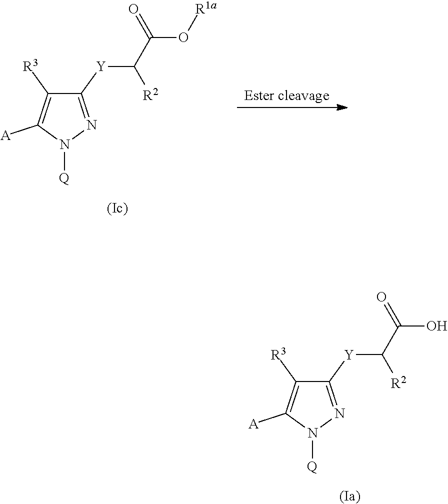
in which R², R^{1a}, R³, A, Q and Y are as set forth in claim 1, in the presence of a base or a Lewis acid.

10. A process for preparing compounds of formula (Ib) or an agrochemically acceptable salt thereof according to claim 1 by converting compounds of general formulae (Ia) and (II) 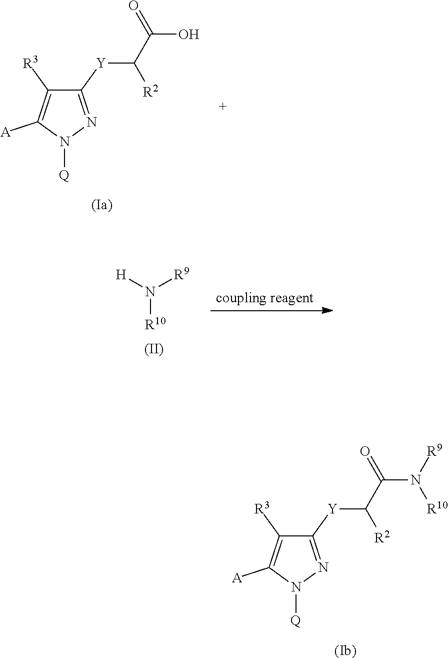
in which R⁹, R¹⁰, R², R^{1a}, R³, A, Q and Y are as set forth in claim 1, in the presence of an amide coupling reagent.

11. An agrochemical composition comprising at least one compound of the formula (I) or an agrochemically acceptable salt thereof as set forth in claim 1.

12. An agrochemical composition comprising:
a) at least one compound of the formula (I) or an agrochemically acceptable salt thereof as set forth in claim 1; and
b) one or more active agrochemical ingredients other than component a).

13. A method of controlling unwanted plants or for regulating the growth of plants, wherein at least one compound of the formula (I) or an agrochemically acceptable salt thereof, as set forth in claim 1, is applied to the plants, a seed or an area in which the plants grow.

14. A method of inhibiting plant growth, the method comprising use of compounds of the formula (I) or an agrochemically acceptable salt thereof, as set forth in claim 1, as herbicides or plant growth regulators.

15. The method according to claim 14, wherein the compounds of the formula (I) or an agrochemically acceptable salt thereof are used for controlling harmful plants or for regulating growth in crop plants.

16. The method according to claim 15, wherein the crop plants are transgenic or nontransgenic crop plants.

17. The compounds according to claim 1, comprising 1-Pyridyl-5-azinylpyrazolyl-3-oxyalkyl acids, 1-pyridyl-5-azinylpyrazolyl-3-sulfanylalkyl acids, 1-pyridyl-5-azinylpyrazolyl-3-sulfinylalkyl acids, 1-pyridyl-5-azinylpyrazolyl-3-sulfonylalkyl acids, or combinations thereof.

18. The process according to claim 8, wherein the sulfurizing reagent is one of phosphorus pentasulfide and Lawesson's reagent.
